(19) United States
(12) Patent Application Publication
(10) Pub. No.: US 2026/0355239 A1
(43) Pub. Date: Nov. 12, 2026

(54) COMBINATION OF A T CELL THERAPY AND CONTINUOUS OR INTERMITTENT DGK INHIBITOR DOSING

(71) Applicant: Juno Therapeutics, Inc., Seattle, WA (US)

(72) Inventors: David G. KUGLER, Seattle, WA (US); Jim QIN, Renton, WA (US); Alison MAIER, Seattle, WA (US); Michael WICHROSKI, Princeton, NJ (US); Pallavur SIVAKUMAR, Summit, NJ (US); Stephanie BUSCH, Seattle, WA (US); Jon JONES, Burien, WA (US); Christopher Mark HILL, Seattle, WA (US); Patrick Andrew CARLSON, Seattle, WA (US)

(73) Assignee: Juno Therapeutics, Inc., Seattle, WA (US)

(21) Appl. No.: 19/110,016

(22) PCT Filed: Sep. 7, 2023
(86) PCT No.: PCT/US2023/073686

§ 371 Date: Mar. 7, 2025

Related U.S. Application Data
(60) Provisional application No. 63/404,955, filed on Sep. 8, 2022.

(51) Int. Cl. A61K 35/17; A61K 31/496; A61K 31/519; A61K 40/11; A61K 40/31; A61K 40/32; A61K 40/42; A61P 35/02

(52) U.S. Cl. CPC A61K 35/17; A61K 31/496; A61K 31/519; A61K 40/11; A61K 40/31; A61K 40/32; A61K 40/4211; A61P 35/02; A61K 2239/28; A61K 2239/48

(57) ABSTRACT

Provided herein are methods, compositions, and uses involving T cell therapies, such as adoptive T cell therapy, and an inhibitor of a Diacylglycerol kinase (DGK). The provided methods, compositions, and uses include those involving the administration or use of one or more DGK inhibitors (DGKi) in a combination therapy with a T cell therapy, such as a genetically engineered T cell therapy involving cells engineered with a recombinant receptor, such as chimeric antigen receptor (CAR) or T cell receptor (TCR).

### CROSS-REFERENCE TO RELATED APPLICATIONS

This application claims priority to U.S. Provisional Application No. 63/404,955, filed Sep. 8, 2022, the contents of which are hereby incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 735042026540SeqList.xml, created Aug. 23, 2023, which is 358 kilobytes in size. The information in the electronic format of the Sequence Listing is incorporated by reference in its entirety.

### FIELD

The present disclosure relates in some aspects to methods, compositions, and uses involving T cell therapies, such as adoptive T cell therapy, and an inhibitor of a Diacylglycerol kinase (DGK). The provided methods, compositions, and uses include those involving the administration or use of one or more DGK inhibitors (DGKi) in a combination therapy with a T cell therapy, such as a genetically engineered T cell therapy involving cells engineered with a recombinant receptor, such as chimeric antigen receptor (CAR) or T cell receptor (TCR).

### BACKGROUND

Various strategies are available for T cell therapies, for example administering engineered T cells for adoptive therapy. For example, strategies are available for engineering T cells expressing genetically engineered antigen receptors, such as CARs or TCRs, and administering compositions containing such cells to subjects. Improved strategies are needed to improve efficacy of the T cells of the cell therapy, for example improving the persistence, activity, and/or proliferation of the cells upon or following administration to subjects. Provided are methods, compositions, uses, and kits that meet such needs.

### SUMMARY

Provided herein in some embodiments is a method of treatment, the method comprising administering to a subject having a cancer a combination therapy comprising: (a) a T cell therapy comprising viable engineered T cells expressing a recombinant receptor directed against an antigen expressed by cells of the cancer, wherein the T cell therapy is administered on Day 1 of the combination therapy; and (b) an inhibitor of DGKα and/or DGKζ, wherein the administration of the inhibitor is initiated on any of Days 1-8, inclusive, of the combination therapy, and the inhibitor is administered continuously at least until the number of engineered T cells of the T cell therapy has peaked in the subject following administration of the T cell therapy.

In some of any embodiments, the administration of the inhibitor is initiated subsequent to the administration of the T cell therapy.

Also provided herein in some embodiments is a method of treatment, the method comprising administering to a subject having a cancer an inhibitor of DGKα and/or DGKζ, wherein the inhibitor is administered as part of a combination therapy comprising the inhibitor and a T cell therapy comprising viable engineered T cells expressing a recombinant receptor directed against an antigen expressed by cells of the cancer, wherein: prior to initiation of administration of the inhibitor, the subject has been previously administered the T cell therapy, wherein the T cell therapy is administered on Day 1 of the combination therapy; the administration of the inhibitor is initiated on any of Days 1-8, inclusive, of the combination therapy; and the inhibitor is administered continuously at least until the number of engineered T cells of the T cell therapy has peaked in the subject following administration of the T cell therapy.

In some of any embodiments, the administration of the inhibitor is initiated on Day 1 of the combination therapy prior to the administration of the T cell therapy.

Also provided herein in some embodiments is a method of treatment, the method comprising administering to a subject having a cancer a T cell therapy comprising viable engineered T cells expressing a recombinant receptor directed against an antigen expressed by cells of the cancer, wherein the T cell therapy is administered as part of a combination therapy comprising the T cell therapy and an inhibitor of DGKα and/or DGKζ, wherein: the T cell therapy is administered on Day 1 of the combination therapy; the administration of the inhibitor is initiated on Day 1 of the combination therapy prior to the administration of the T cell therapy; and the inhibitor is administered continuously at least until the number of engineered T cells of the T cell therapy has peaked in the subject following administration of the T cell therapy.

In some of any embodiments, the inhibitor is administered daily, once every two days, or once every three days. In some of any embodiments, the inhibitor is administered daily.

In some of any embodiments, the inhibitor is administered once on each of the days that the inhibitor is administered.

In some of any embodiments, the inhibitor is administered until the number of engineered T cells of the T cell therapy has peaked in the subject following administration of the T cell therapy.

In some of any embodiments, the inhibitor is administered over a period of time between or between about 21 and 42 days, inclusive.

Also provided herein in some embodiments is a method of treatment, the method comprising administering to a subject having a cancer a combination therapy comprising: (a) a T cell therapy comprising viable engineered T cells expressing a recombinant receptor directed against an antigen expressed by cells of the cancer, wherein the T cell therapy is administered on Day 1 of the combination therapy; and (b) an inhibitor of DGKα and/or DGKζ, wherein the administration of the inhibitor is initiated on any of Days 1-8, inclusive, of the combination therapy, and the inhibitor is administered in a therapeutically effective dose for a period between or between about 21 and 42 days.

In some of any embodiments, the administration of the inhibitor is initiated subsequent to the administration of the T cell therapy.

Also provided herein in some embodiments is a method of treatment, the method comprising administering to a subject having a cancer an inhibitor of DGKα and/or DGKζ, wherein the inhibitor is administered as part of a combination therapy comprising the inhibitor and a T cell therapy comprising viable engineered T cells expressing a recombinant receptor directed against an antigen expressed by cells of the cancer, wherein: prior to initiation of administration of the inhibitor, the subject has been previously administered the T cell therapy, wherein the T cell therapy is administered on Day 1 of the combination therapy; the administration of the inhibitor is initiated on any of Days 1-8, inclusive, of the combination therapy; and the inhibitor is administered in a therapeutically effective dose for a period between or between about 21 and 42 days.

In some of any embodiments, the administration of the inhibitor is initiated on Day 1 of the combination therapy prior to the administration of the T cell therapy.

Also provided herein in some embodiments is a method of treatment, the method comprising administering to a subject having a cancer a T cell therapy comprising viable engineered T cells expressing a recombinant receptor directed against an antigen expressed by cells of the cancer, wherein the T cell therapy is administered as part of a combination therapy comprising the T cell therapy and an inhibitor of DGKα and/or DGKζ, wherein: the T cell therapy is administered on Day 1 of the combination therapy; the administration of the inhibitor is initiated on Day 1 of the combination therapy prior to the administration of the T cell therapy; and the inhibitor is administered in a therapeutically effective dose for a period between or between about 21 and 42 days.

In some of any embodiments, the therapeutically effective dose provides a dose of the inhibitor that is continuously therapeutically effective for the period between or between about 21 and 42 days.

In some of any embodiments, the inhibitor is administered every 2 days or 3 days.

Also provided herein in some embodiments is a method of treatment, the method comprising administering to a subject having a cancer a combination therapy comprising: (a) a T cell therapy comprising viable engineered T cells expressing a recombinant receptor directed against an antigen expressed by cells of the cancer, wherein the T cell therapy is administered on Day 1 of the combination therapy; and (b) an inhibitor of DGKα and/or DGKζ, wherein the administration of the inhibitor is initiated on any of Days 1-8, inclusive, of the combination therapy, and the inhibitor is administered in a plurality of cycles each comprising an administration period in which the inhibitor is administered followed by a rest period during which the inhibitor is not administered.

In some of any embodiments, the administration of the inhibitor is initiated subsequent to the administration of the T cell therapy.

Also provided herein in some embodiments is a method of treatment, the method comprising administering to a subject having a cancer an inhibitor of DGKα and/or DGKζ, wherein the inhibitor is administered as part of a combination therapy comprising the inhibitor and a T cell therapy comprising viable engineered T cells expressing a recombinant receptor directed against an antigen expressed by cells of the cancer, wherein: prior to initiation of administration of the inhibitor, the subject has been previously administered the T cell therapy, wherein the T cell therapy is administered on Day 1 of the combination therapy; the administration of the inhibitor is initiated on any of Days 1-8, inclusive, of the combination therapy; and the inhibitor is administered in a plurality of cycles each comprising an administration period in which the inhibitor is administered followed by a rest period during which the inhibitor is not administered.

In some of any embodiments, during the administration period, the inhibitor is administered daily, every two days, or every three days. In some of any embodiments, during the administration period, the inhibitor is administered daily.

In some of any embodiments, during the administration period, the inhibitor is administered once on each of the days that the inhibitor is administered.

In some of any embodiments, over the plurality of cycles, the inhibitor is administered until the number of engineered T cells of the T cell therapy has peaked in the subject following administration of the T cell therapy.

In some of any embodiments, over the plurality of cycles, the inhibitor is administered over a period of time between or between about 21 and 42 days, inclusive.

Also provided herein in some embodiments is a method of treatment, the method comprising administering to a subject having a cancer a combination therapy comprising: (a) a T cell therapy comprising viable engineered T cells expressing a recombinant receptor directed against an antigen expressed by cells of the cancer, wherein the T cell therapy is administered on Day 1 of the combination therapy; and (b) an inhibitor of DGKα and/or DGKζ, wherein the administration of the inhibitor is initiated on any of Days 1-8, inclusive, of the combination therapy, and the inhibitor is administered once daily for between or between about 21 and 42 consecutive days, inclusive.

In some of any embodiments, the administration of the inhibitor is initiated subsequent to the administration of the T cell therapy.

Also provided herein in some embodiments is a method of treatment, the method comprising administering to a subject having a cancer an inhibitor of DGKα and/or DGKζ, wherein the inhibitor is administered as part of a combination therapy comprising the inhibitor and a T cell therapy comprising viable engineered T cells expressing a recombinant receptor directed against an antigen expressed by cells of the cancer, wherein: prior to initiation of administration of the inhibitor, the subject has been previously administered the T cell therapy, wherein the T cell therapy is administered on Day 1 of the combination therapy; the administration of the inhibitor is initiated on any of Days 1-8, inclusive, of the combination therapy; and the inhibitor is administered once daily for between or between about 21 and 42 consecutive days, inclusive.

In some of any embodiments, the administration of the inhibitor is initiated on any of Days 1-6, inclusive, of the combination therapy. In some of any embodiments, the administration of the inhibitor is initiated on any of Days 1-4, inclusive, of the combination therapy. In some of any embodiments, the administration of the inhibitor is initiated on Day 1 or Day 2 of the combination therapy. In some of any embodiments, the administration of the inhibitor is initiated on Day 1 of the combination therapy.

In some of any embodiments, the administration of the inhibitor is initiated on Day 1 of the combination therapy prior to the administration of the T cell therapy.

Also provided herein in some embodiments is a method of treatment, the method comprising administering to a subject having a cancer a T cell therapy comprising viable engineered T cells expressing a recombinant receptor directed against an antigen expressed by cells of the cancer, wherein the T cell therapy is administered as part of a combination therapy comprising the T cell therapy and an inhibitor of DGKα and/or DGKζ, wherein: the T cell therapy is administered on Day 1 of the combination therapy; the administration of the inhibitor is initiated on Day 1 of the combination therapy prior to the administration of the T cell therapy; and the inhibitor is administered once daily for between or between about 21 and 42 consecutive days, inclusive.

In some of any embodiments, the inhibitor is administered over a period of time between or between about 21 and 35 days, inclusive. In some of any embodiments, the inhibitor is administered over a period of time between or between about 21 and 28 days, inclusive. In some of any embodiments, the inhibitor is administered over a period of time between or between about 28 and 42 days, inclusive. In some of any embodiments, the inhibitor is administered over a period of time between or between about 28 and 35 days, inclusive. In some of any embodiments, the inhibitor is administered over a period of time that is or is about 28 days. In some of any embodiments, the inhibitor is administered over a period of time that is or is about 30 days.

In some of any embodiments, over the plurality of cycles, the inhibitor is administered over a period of time between or between about 21 and 35 days, inclusive. In some of any embodiments, over the plurality of cycles, the inhibitor is administered over a period of time between or between about 21 and 28 days, inclusive. In some of any embodiments, over the plurality of cycles, the inhibitor is administered over a period of time between or between about 28 and 42 days, inclusive. In some of any embodiments, over the plurality of cycles, the inhibitor is administered over a period of time between or between about 28 and 35 days, inclusive. In some of any embodiments, over the plurality of cycles, the inhibitor is administered over a period of time that is or is about 28 days. In some of any embodiments, over the plurality of cycles, the inhibitor is administered over a period of time that is or is about 30 days.

In some of any embodiments, the inhibitor is administered once daily for between or between about 21 and 35 consecutive days, inclusive. In some of any embodiments, the inhibitor is administered once daily for between or between about 21 and 28 consecutive days, inclusive. In some of any embodiments, the inhibitor is administered once daily for between or between about 28 and 42 consecutive days, inclusive. In some of any embodiments, the inhibitor is administered once daily for between or between about 28 and 35 consecutive days, inclusive. In some of any embodiments, the inhibitor is administered once daily for or for about 28 consecutive days.

Also provided herein in some embodiments is a method of treatment, the method comprising administering to a subject having a cancer a combination therapy comprising: (a) a T cell therapy comprising viable engineered T cells expressing a recombinant receptor directed against an antigen expressed by cells of the cancer, wherein the T cell therapy is administered on Day 1 of the combination therapy; and (b) an inhibitor of DGKα and/or DGKζ, wherein the administration of the inhibitor is initiated on Day 1 of the combination therapy, and the inhibitor is administered once daily on Days 1-28, inclusive, of the combination therapy.

In some of any embodiments, the administration of the inhibitor is initiated subsequent to the administration of the T cell therapy.

Also provided herein in some embodiments is a method of treatment, the method comprising administering to a subject having a cancer an inhibitor of DGKα and/or DGKζ, wherein the inhibitor is administered as part of a combination therapy comprising the inhibitor and a T cell therapy comprising viable engineered T cells expressing a recombinant receptor directed against an antigen expressed by cells of the cancer, wherein: prior to initiation of administration of the inhibitor, the subject has been previously administered the T cell therapy, wherein the T cell therapy is administered on Day 1 of the combination therapy; the administration of the inhibitor is initiated on Day 1 of the combination therapy; and the inhibitor is administered once daily on Days 1-28, inclusive, of the combination therapy.

In some of any embodiments, the administration of the inhibitor is initiated prior to the administration of the T cell therapy.

Also provided herein in some embodiments is a method of treatment, the method comprising administering to a subject having a cancer a T cell therapy comprising viable engineered T cells expressing a recombinant receptor directed against an antigen expressed by cells of the cancer, wherein the T cell therapy is administered as part of a combination therapy comprising the T cell therapy and an inhibitor of DGKα and/or DGKζ, wherein: the T cell therapy is administered on Day 1 of the combination therapy; the administration of the inhibitor is initiated on Day 1 of the combination therapy prior to the administration of the T cell therapy; and the inhibitor is administered once daily on Days 1-28, inclusive, of the combination therapy.

In some of any embodiments, the inhibitor is administered once daily on Days 1-30, inclusive, of the combination therapy.

In some of any embodiments, the administration of the inhibitor is initiated within or within about 12 hours of the administration of the T cell therapy. In some of any embodiments, the administration of the inhibitor is initiated within or within about 6 hours of the administration of the T cell therapy. In some of any embodiments, the administration of the inhibitor is initiated within or within about 4 hours of the administration of the T cell therapy. In some of any embodiments, the administration of the inhibitor is initiated within or within about 2 hours of the administration of the T cell therapy. In some of any embodiments, the administration of the inhibitor is initiated within or within about 1 hour of the administration of the T cell therapy.

In some of any embodiments, the administration of the inhibitor is initiated on Day 1 of the combination therapy concurrently with the administration of the T cell therapy. In some of any embodiments, the administration of the inhibitor is initiated on Day 1 of the combination therapy during the administration of the T cell therapy.

In some of any embodiments, prior to the administration of the T cell therapy, the subject has been preconditioned with a lymphodepleting therapy comprising administration of fludarabine and/or cyclophosphamide. In some of any embodiments, prior to the administration of the T cell therapy, the subject has been preconditioned with a lymphodepleting therapy comprising administration of fludarabine and cyclophosphamide. In some of any embodiments, the method further comprises administering the lymphodepleting therapy to the subject.

In some of any embodiments, the lymphodepleting therapy comprises administration of: cyclophosphamide at between or between about 200 and 400 mg/m², inclusive, daily for between or between about 2 and 4 days, inclusive; and/or fludarabine at between or between about 20 and 40 mg/m², inclusive, daily for between or between about 2 and 4 days, inclusive. In some of any embodiments, the lymphodepleting therapy comprises administration of cyclophosphamide at between or between about 200 and 400 mg/m², inclusive, daily for between or between about 2 and 4 days, inclusive. In some of any embodiments, the lymphodepleting therapy comprises administration of fludarabine at between or between about 20 and 40 mg/m², inclusive, daily for between or between about 2 and 4 days, inclusive. In some of any embodiments, the lymphodepleting therapy comprises administration of cyclophosphamide at or at about 300 mg/m² and fludarabine at or at about 30 mg/m² each daily for or for about 3 days.

In some of any embodiments, the T cell therapy is administered between or between about 2 and 7 days, inclusive, after the administration of the lymphodepleting therapy.

In some of any embodiments, the inhibitor is an inhibitor of DGKα and not a significant inhibitor of DGKζ. In some of any embodiments, the inhibitor is an inhibitor of DGKζ and not a significant inhibitor of DGKα. In some of any embodiments, the inhibitor is an inhibitor of DGKα and DGKζ.

In some of any embodiments, the inhibitor is not a significant inhibitor of other DGKs.

In some of any embodiments, the inhibitor is a compound of Formula (I):

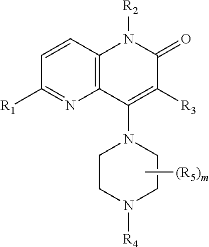

or a pharmaceutically acceptable salt thereof, wherein:
R₁ is H, F, Cl, Br, —CN, C₁₋₃ alkyl substituted with zero to 4 R₁ₐ, C₃₋₄ cycloalkyl substituted with zero to 4 R₁ₐ, C₁₋₃ alkoxy substituted with zero to 4 R₁ₐ, —NRₐRₐ, —S(O)ₙRₑ, or —P(O)RₑRₑ;
each R₁ₐ is independently F, Cl, —CN, —OH, —OCH₃, or —NRₐRₐ;
each Rₐ is independently H or C₁₋₃ alkyl;
each Rₑ is independently C₃₋₄ cycloalkyl or C₁₋₃ alkyl substituted with zero to 4 R₁ₐ;
R₂ is H, C₁₋₃ alkyl substituted with zero to 4 R₂ₐ, or C₃₋₄ cycloalkyl substituted with zero to 4 R₂ₐ;
each R₂ₐ is independently F, Cl, —CN, —OH, —O(C₁₋₂ alkyl), C₃₋₄ cycloalkyl, C₃₋₄ alkenyl, or C₃₋₄ alkynyl;
R₃ is H, F, Cl, Br, —CN, C₁₋₃ alkyl, C₁₋₂ fluoroalkyl, C₃₋₄ cycloalkyl, C₃₋₄ fluorocycloalkyl, or —NO₂;
R₄ is —CH₂R₄ₐ, —CH₂CH₂R₄ₐ, —CH₂CHR₄ₐR_{4d}, —CHR₄ₐR_{4b}, or —CR₄ₐR_{4b}R_{4c};
R₄ₐ and R_{4b} are independently:
(i) C₁₋₆ alkyl substituted with zero to 4 substituents independently selected from F, Cl, —CN, —OH, —OCH₃, —SCH₃, C₁₋₃ fluoroalkoxy, —NRₐRₐ, —S(O)₂Rₑ, or —NRₐS(O)₂Rₑ;
(ii) C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, C₁₋₄ hydroxyalkyl, —(CH₂)₁₋₂O(C₁₋₃ alkyl), C₁₋₄ alkoxy, —O(C₁₋₄ hydroxyalkyl), —O(CH)₁₋₃O(C₁₋₃ alkyl), C₁₋₃ fluoroalkoxy, —O(CH)₁₋₃NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₃ alkyl)₂, —S(O)₂(C₁₋₃ alkyl), —O(CH₂)₁₋₂(C₃₋₆ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or
(iii) C₁₋₄ alkyl substituted with one cyclic group selected from C₃₋₆ cycloalkyl, heterocyclyl, aryl, and heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl;
or R₄ₐ and R_{4b} together with the carbon atom to which they are attached form a C₃₋₆ cycloalkyl or a 3- to 6-membered heterocyclyl, each substituted with zero to 3 R_{f};
each R_{f} is independently F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, and —NR_{c}R_{c};
R_{4c} is C₁₋₆ alkyl or C₃₋₆ cycloalkyl, each substituted with zero to 4 substituents independently selected from F, Cl, —OH, C₁₋₂ alkoxy, C₁₋₂ fluoroalkoxy, and —CN;
R_{4d} is —OCH₃;
each R_{c} is independently H or C₁₋₂ alkyl;
R_{d} is phenyl substituted with zero to 1 substituent selected from F, Cl, —CN, —CH₃, and —OCH₃;
each R₅ is independently —CN, C₁₋₆ alkyl substituted with zero to 4 R_{g}, C₂₋₄ alkenyl substituted with zero to 4 R_{g}, C₂₋₄ alkynyl substituted with zero to 4 R_{g}, C₃₋₄ cycloalkyl substituted with zero to 4 R_{g}, phenyl substituted with zero to 4 R_{g}, oxadiazolyl substituted with zero to 3 R_{g}, pyridinyl substituted with zero to 4 R_{g}, —(CH₂)₁₋₂(heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each R_{g} is independently F, Cl, —CN, —OH, C₁₋₃ alkoxy, C₁₋₃ fluoroalkoxy, —O(CH₂)₁₋₂O(C₁₋₂ alkyl), or —NR_{c}R_{c};
m is zero, 1, 2, or 3; and
n is zero, 1, or 2.

In some of any embodiments, the inhibitor is a compound of Formula (I) or a pharmaceutically acceptable salt thereof, wherein:
R₁ is H, F, Cl, Br, —CN, C₁₋₃ alkyl substituted with zero to 4 R₁ₐ, cyclopropyl substituted with zero to 3 R₁ₐ, C₁₋₃ alkoxy substituted with zero to 3 R₁ₐ, —NRₐRₐ, —S(O)ₙCH₃, or —P(O)(CH₃)₂;
each R₁ₐ is independently F, Cl, or —CN;
each Rₐ is independently H or C₁₋₃ alkyl;
R₂ is H or C₁₋₂ alkyl substituted with zero to 2 R₂ₐ,
each R₂ₐ is independently F, Cl, —CN, —OH, —O(C₁₋₂ alkyl), cyclopropyl, C₃₋₄ alkenyl, or C₃₋₄ alkynyl;
R₃ is H, F, Cl, Br, —CN, C₁₋₂ alkyl, —CF₃, cyclopropyl, or —NO₂;
R₄ₐ and R_{4b} are independently:
(i) C₁₋₄ alkyl substituted with zero to 4 substituents independently selected from F, Cl, —CN, —OH, —OCH₃, —SCH₃, C₁₋₃ fluoroalkoxy, and —NRₐRₐ;
(ii) C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, —CH₂OH, —(CH₂)₁₋₂O(C₁₋₂ alkyl), C₁₋₄ alkoxy, —O(C₁₋₄ hydroxyalkyl), —O(CH)₁₋₂O(C₁₋₂ alkyl), C₁₋₃ fluoroalkoxy, —O(CH)₁₋₂NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —O(CH₂)₁₋₂(C₃₋₄ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or
(iii) C₁₋₃ alkyl substituted with one cyclic group selected from C₃₋₆ cycloalkyl, heterocyclyl, phenyl, and heteroaryl, said cyclic group substituted with zero to 3 substituents independently selected from F, Cl, Br, —OH, —CN, C₁₋₃ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₄ cycloalkyl;
or R₄ₐ and R_{4b} together with the carbon atom to which they are attached, form a C₃₋₆ cycloalkyl or a 3- to 6-membered heterocyclyl, each substituted with zero to 3 R_{f};
each R_{f} is independently F, Cl, Br, —OH, —CN, C₁₋₄ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₄ alkyl, C₁₋₂ fluoroalkyl, C₁₋₃ alkoxy, C₁₋₂ fluoroalkoxy, and —NR_{c}R_{c};
R_{4c} is C₁₋₄ alkyl or C₃₋₆ cycloalkyl, each substituted with zero to 4 substituents independently selected from F, Cl, —OH, C₁₋₂ alkoxy, C₁₋₂ fluoroalkoxy, and —CN;
and each R₅ is independently —CN, C₁₋₅ alkyl substituted with zero to 4 R_{g}, C₂₋₃ alkenyl substituted with zero to 4 R_{g}, C₂₋₃ alkynyl substituted with zero to 4 R_{g}, C₃₋₄ cycloalkyl substituted with zero to 4 R_{g}, phenyl substituted with zero to 3 R_{g}, oxadiazolyl substituted with zero to 3 R_{g}, pyridinyl substituted with zero to 3 R_{g}, —(CH₂)₁₋₂(heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl).

In some of any embodiments, the inhibitor is a compound of Formula (I) or a pharmaceutically acceptable salt thereof having the structure:

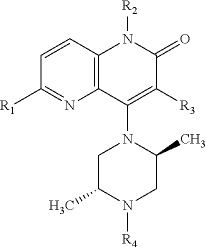

wherein:
R₁ is —CN;
R₂ is —CH₃;
R₃ is H, F, or —CN;
R₄ is:

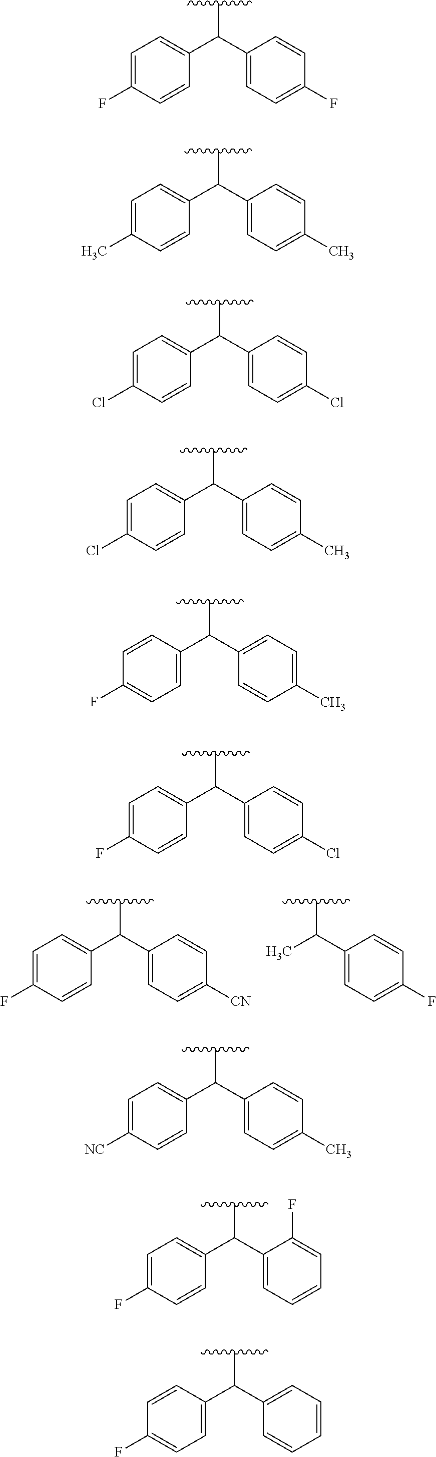

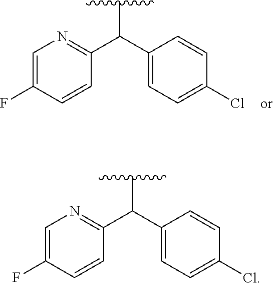 [SMILES: *C(c1ccc(Cl)cc1)c1ccc(F)cn1 ; *C(c1ccc(Cl)cc1)c1ccc(F)cn1]

In some of any embodiments, the inhibitor is a compound of Formula (I) or a pharmaceutically acceptable salt thereof having the structure:

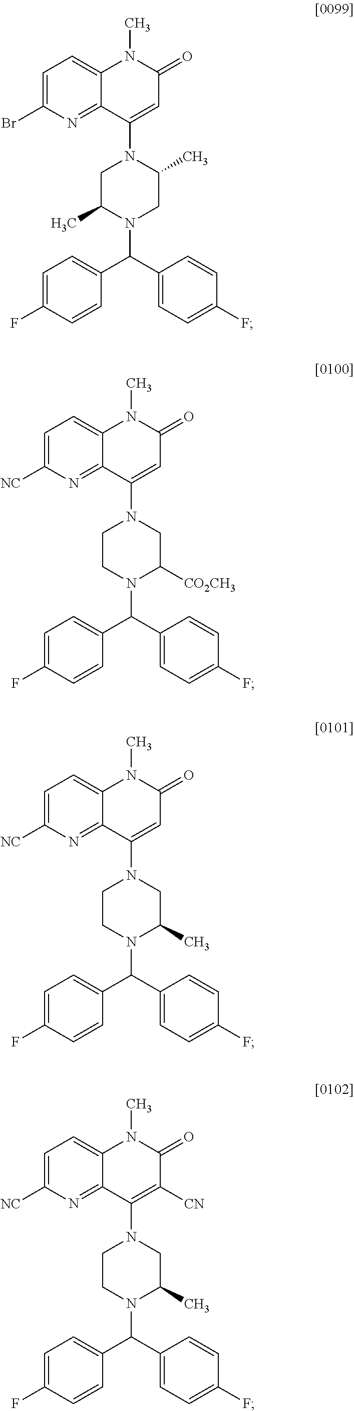 [SMILES: C[C@@H]1CN(C(c2ccc(F)cc2)c2ccc(F)cc2)[C@@H](C)CN1c1cc(=O)n(C)c2ccc(Br)nc12 ; COC(=O)C1CN(c2cc(=O)n(C)c3ccc(C#N)nc23)CCN1C(c1ccc(F)cc1)c1ccc(F)cc1 ; C[C@@H]1CN(c2cc(=O)n(C)c3ccc(C#N)nc23)CCN1C(c1ccc(F)cc1)c1ccc(F)cc1 ; C[C@@H]1CN(c2c(C#N)c(=O)n(C)c3ccc(C#N)nc23)CCN1C(c1ccc(F)cc1)c1ccc(F)cc1]

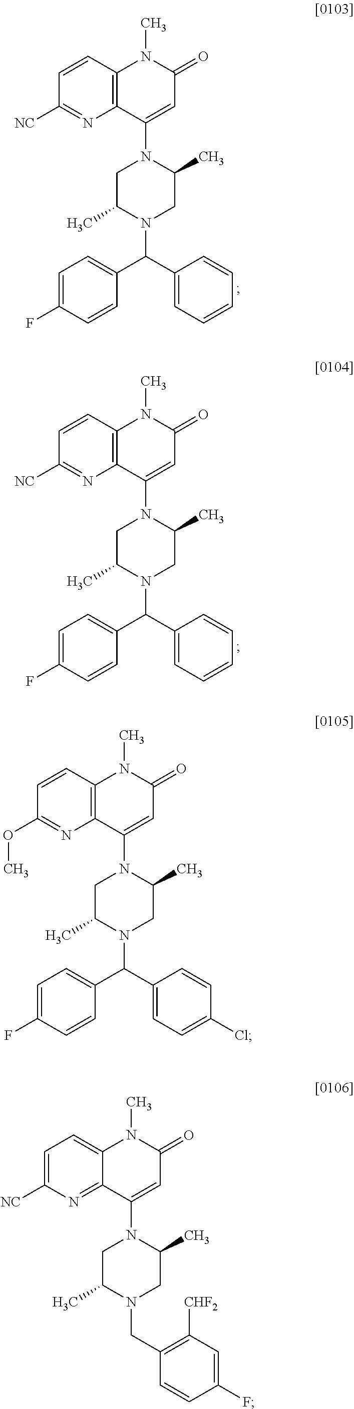 [SMILES: C[C@@H]1CN(c2cc(=O)n(C)c3ccc(C#N)nc23)[C@@H](C)CN1C(c1ccccc1)c1ccc(F)cc1 ; C[C@@H]1CN(c2cc(=O)n(C)c3ccc(C#N)nc23)[C@@H](C)CN1C(c1ccccc1)c1ccc(F)cc1 ; COc1ccc2c(n1)c(N1C[C@@H](C)N(C(c3ccc(F)cc3)c3ccc(Cl)cc3)C[C@@H]1C)cc(=O)n2C ; C[C@@H]1CN(c2cc(=O)n(C)c3ccc(C#N)nc23)[C@@H](C)CN1Cc1ccc(F)cc1C(F)F]

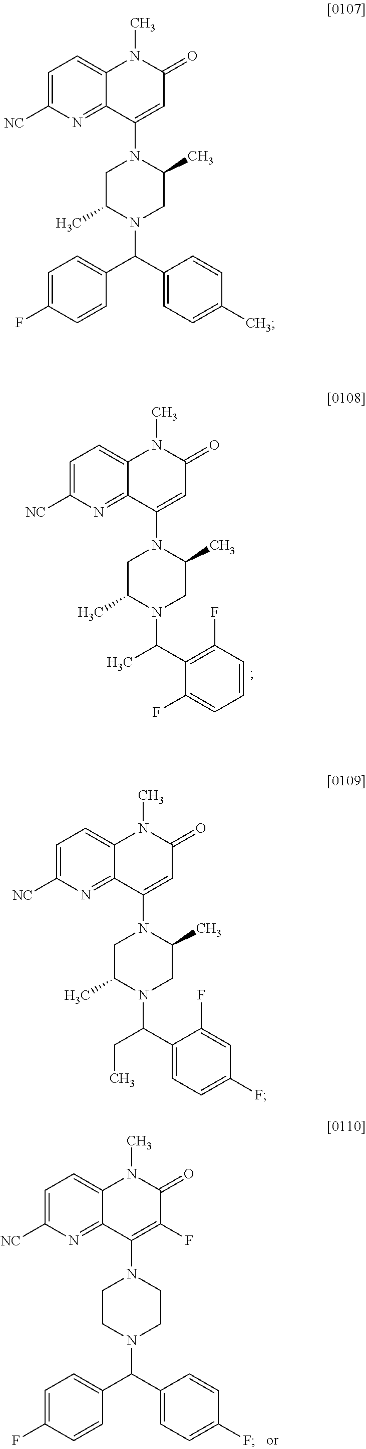 [SMILES: Cc1ccc(C(c2ccc(F)cc2)N2C[C@H](C)N(c3cc(=O)n(C)c4ccc(C#N)nc34)C[C@H]2C)cc1 ; CC(c1c(F)cccc1F)N1C[C@H](C)N(c2cc(=O)n(C)c3ccc(C#N)nc23)C[C@H]1C ; CCC(c1ccc(F)cc1F)N1C[C@H](C)N(c2cc(=O)n(C)c3ccc(C#N)nc23)C[C@H]1C ; Cn1c(=O)c(F)c(N2CCN(C(c3ccc(F)cc3)c3ccc(F)cc3)CC2)c2nc(C#N)ccc21]

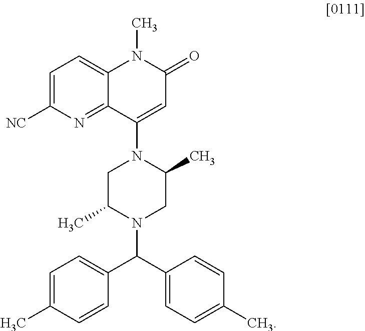 [SMILES: Cc1ccc(C(c2ccc(C)cc2)N2C[C@H](C)N(c3cc(=O)n(C)c4ccc(C#N)nc34)C[C@H]2C)cc1]

In some of any embodiments, the inhibitor is a compound of Formula (II):

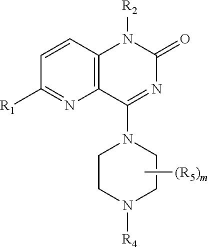

or a salt thereof, wherein:
R₁ is H, F, Cl, Br, —CN, —OH, C₁₋₃ alkyl substituted with zero to 4 R₁ₐ, C₃₋₄ cycloalkyl substituted with zero to 4 R₁ₐ, C₁₋₃ alkoxy substituted with zero to 4 R₁ₐ, —NRₐRₐ, —S(O)ₙRₑ, or —P(O)RₑRₑ;
each R₁ₐ is independently F, Cl, —CN, —OH, —OCH₃, or —NRₐRₐ;
each Rₐ is independently H or C₁₋₃ alkyl;
each Rₑ is independently C₃₋₄ cycloalkyl or C₁₋₃ alkyl substituted with zero to 4 R₁ₐ;
R₂ is H, C₁₋₃ alkyl substituted with zero to 4 R₂ₐ, or C₃₋₄ cycloalkyl substituted with zero to 4 R₂ₐ;
each R₂ₐ is independently F, Cl, —CN, —OH, —O(C₁₋₂ alkyl), C₃₋₄ cycloalkyl, C₃₋₄ alkenyl, or C₃₋₄ alkynyl;
R₄ is —CH₂R₄ₐ, —CH₂CH₂R₄ₐ, —CH₂CHR₄ₐR_{4d}, —CHR₄ₐR_{4b}, or —CR₄ₐR_{4b}R_{4c};
R₄ₐ and R_{4b} are independently:
(i) —CN or C₁₋₆ alkyl substituted with zero to 4 substituents independently selected from F, Cl, —CN, —OH, —OCH₃, —SCH₃, C₁₋₃ fluoroalkoxy, —NRₐRₐ, —S(O)₂Rₑ, or —NRₐS(O)₂Rₑ;
(ii) C₃₋₆ cycloalkyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₄ hydroxyalkyl, —(CH₂)₁₋₂O(C₁₋₃ alkyl), C₁₋₄ alkoxy, C₁₋₃ fluoroalkoxy, C₁₋₃ cyanoalkoxy, —O(C₁₋₄ hydroxyalkyl), —O(CRₓRₓ)₁₋₃O(C₁₋₃ alkyl), C₁₋₃ fluoroalkoxy, —O(CH₂)₁₋₃NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —CH₂NRₐRₐ, —NRₐS(O)₂(C₁₋₃ alkyl), —NRₐC(O)(C₁₋₃ alkyl), —(CRₓRₓ)₀₋₂NRₐC(O)O(C₁₋₄ alkyl), —P(O)(C₁₋₃ alkyl)₂, —S(O)₂(C₁₋₃ alkyl), —(CRₓRₓ)₁₋₂(C₃₋₄ cycloalkyl), —(CRₓRₓ)₁₋₂(morpholinyl), —(CRₓRₓ)₁₋₂(difluoromorpholinyl), —(CRₓRₓ)₁₋₂(dimethylmorpholinyl), —(CRₓRₓ)₁₋₂(oxaazabicyclo[2.2.1]heptanyl), (CRₓRₓ)₁₋₂(oxaazaspiro[3.3]heptanyl), —(CRₓRₓ)₁₋₂(methylpiperazinonyl), —(CRₓRₓ)₁₋₂(acetylpiperazinyl), —(CRₓRₓ)₁₋₂(piperidinyl), —(CRₓRₓ)₁₋₂(difluoropiperidinyl), —(CRₓRₓ)₁₋₂(methoxypiperidinyl), —(CRₓRₓ)₁₋₂(hydroxypiperidinyl), —O(CRₓRₓ)₀₋₂(C₃₋₆ cycloalkyl), —O(CRₓRₓ)₀₋₂(methylcyclopropyl), —O(CRₓRₓ)₀₋₂((ethoxycarbonyl)cyclopropyl), —O(CRₓRₓ)₀₋₂(oxetanyl), —O(CRₓRₓ)₀₋₂(methylazetidinyl), —O(CRₓRₓ)₀₋₂(tetrahydropyranyl), —O(CRₓRₓ)₁₋₂(morpholinyl), —O(CRₓRₓ)₀₋₂(thiazolyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, dioxolanyl, pyrrolidinonyl, and R_{d}; or
(iii) C₁₋₄ alkyl substituted with one cyclic group selected from C₃₋₆ cycloalkyl, 4- to 10-membered heterocyclyl, mono- or bicyclic aryl, or 5- to 10-membered heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl;
or R₄ₐ and R_{4b} together with the carbon atom to which they are attached form a C₃₋₆ cycloalkyl or a 3- to 6-membered heterocyclyl, each substituted with zero to 3 R_{f};
each R_{f} is independently F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, and —NR_{c}R_{c};
R_{4c} is C₁₋₆ alkyl or C₃₋₆ cycloalkyl, each substituted with zero to 4 substituents independently selected from F, Cl, —OH, C₁₋₂ alkoxy, C₁₋₂ fluoroalkoxy, and —CN;
R_{4d} is —OCH₃;
each R_{c} is independently H or C₁₋₂ alkyl;
R_{d} is phenyl substituted with zero to 1 substituent selected from F, Cl, —CN, —CH₃, and —OCH₃;
each R₅ is independently —CN, C₁₋₆ alkyl substituted with zero to 4 R_{g}, C₂₋₄ alkenyl substituted with zero to 4 R_{g}, C₂₋₄ alkynyl substituted with zero to 4 R_{g}, C₃₋₄ cycloalkyl substituted with zero to 4 R_{g}, phenyl substituted with zero to 4 R_{g}, oxadiazolyl substituted with zero to 3 R_{g}, pyridinyl substituted with zero to 4 R_{g}, —(CH₂)₁₋₂(4- to 10-membered heterocyclyl substituted with zero to 4 R_{g}), —(CH₂)₁₋₂NR_{c}C(O)(C₁₋₄ alkyl), (CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each R_{g} is independently F, Cl, —CN, —OH, C₁₋₃ alkoxy, C₁₋₃ fluoroalkoxy, —O(CH₂)₁₋₂O(C₁₋₂ alkyl), or —NR_{c}R_{c};
m is zero, 1, 2, or 3; and
n is zero, 1, or 2.

In some of any embodiments, the inhibitor is a compound of Formula (II) or a pharmaceutically acceptable salt thereof, wherein:
R₁ is H, F, Cl, Br, —CN, —OH, C₁₋₃ alkyl substituted with zero to 4 R₁ₐ, cyclopropyl substituted with zero to 3 R₁ₐ, C₁₋₃ alkoxy substituted with zero to 3 R₁ₐ, —NRₐRₐ, —S(O)ₙCH₃, or —P(O)(CH₃)₂;
R₂ is H or C₁₋₂ alkyl substituted with zero to 2 R₂ₐ;
each R₂ₐ is independently F, Cl, —CN, —OH, —O(C₁₋₂ alkyl), cyclopropyl, C₃₋₄ alkenyl, or C₃₋₄ alkynyl;
R₄ₐ and R_{4b} are independently:
(i) —CN or C₁₋₄ alkyl substituted with zero to 4 substituents independently selected from F, Cl, —CN, —OH, —OCH₃, —SCH₃, C₁₋₃ fluoroalkoxy, and —NRₐRₐ;
(ii) C₃₋₆ cycloalkyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₂ hydroxyalkyl, —CH₂NRₐRₐ, —(CH₂)₁₋₂O(C₁₋₂ alkyl), —(CH₂)₁₋₂NRₓC(O)O(C₁₋₂ alkyl), C₁₋₄ alkoxy, —O(C₁₋₄ hydroxyalkyl), —O(CRₓRₓ)₁₋₂O(C₁₋₂ alkyl), C₁₋₃ fluoroalkoxy, C₁₋₃ cyanoalkoxy, —O(CH₂)₁₋₂NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NR₂C(O)O(C₁₋₄ alkyl), —P(O)(C₁₋₂ alkyl)₂, —S(O)₂(C₁₋₃ alkyl), —(CH₂)₁₋₂(C₃₋₄ cycloalkyl), —CRₓRₓ(morpholinyl), —CRₓRₓ(difluoromorpholinyl), —CRₓRₓ(dimethylmorpholinyl), —CRₓRₓ(oxaazabicyclo[2.2.1]heptanyl), —CRₓRₓ(oxaazaspiro[3.3]heptanyl), —CRₓRₓ(methylpiperazinonyl), —CRₓRₓ(acetylpiperazinyl), —CRₓRₓ(piperidinyl), —CRₓRₓ(difluoropiperidinyl), —CRₓRₓ(methoxypiperidinyl), —CRₓRₓ(hydroxypiperidinyl), —O(CH₂)₀₋₂(C₃₋₄ cycloalkyl), —O(CH₂)₀₋₂(methylcyclopropyl), —O(CH₂)₀₋₂((ethoxycarbonyl)cyclopropyl), —O(CH₂)₀₋₂(oxetanyl), —O(CH₂)₀₋₂(methylazetidinyl), —O(CH₂)₁₋₂(morpholinyl), —O(CH₂)₀₋₂(tetrahydropyranyl), —O(CH₂)₀₋₂(thiazolyl), cyclopropyl, cyanocyclopropyl, methylazetidinyl, acetylazetidinyl, (tert-butoxycarbonyl)azetidinyl, dioxolanyl, pyrrolidinonyl, triazolyl, tetrahydropyranyl, morpholinyl, thiophenyl, methylpiperidinyl, and R_{d}; or
(iii) C₁₋₃ alkyl substituted with one cyclic group selected from C₃₋₆ cycloalkyl, 4- to 10-membered heterocyclyl, mono- or bicyclic aryl, or 5- to 10-membered heteroaryl, said cyclic group substituted with zero to 3 substituents independently selected from F, Cl, Br, —OH, —CN, C₁₋₃ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₄ cycloalkyl;
or R₄ₐ and R_{4b} together with the carbon atom to which they are attached, form a C₃₋₆ cycloalkyl or a 3- to 6-membered heterocyclyl, each substituted with zero to 3 R_{f};
each R_{f} is independently F, Cl, Br, —OH, —CN, C₁₋₄ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₄ alkyl, C₁₋₂ fluoroalkyl, C₁₋₃ alkoxy, C₁₋₂ fluoroalkoxy, and —NR_{c}R_{c};
R_{4c} is C₁₋₄ alkyl or C₃₋₆ cycloalkyl, each substituted with zero to 4 substituents independently selected from F, Cl, —OH, C₁₋₂ alkoxy, C₁₋₂ fluoroalkoxy, and —CN;
each R₅ is independently —CN, C₁₋₅ alkyl substituted with zero to 4 R_{g}, C₂₋₃ alkenyl substituted with zero to 4 R_{g}, C₂₋₃ alkynyl substituted with zero to 4 R_{g}, C₃₋₄ cycloalkyl substituted with zero to 4 R_{g}, phenyl substituted with zero to 3 R_{g}, oxadiazolyl substituted with zero to 3 R_{g}, pyridinyl substituted with zero to 3 R_{g}, —(CH₂)₁₋₂(4- to 10-membered heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each Rₓ is independently H or —CH₃; and
m is 1, 2, or 3.

In some of any embodiments, the inhibitor is a compound of Formula (II) or a pharmaceutically acceptable salt thereof having the structure:

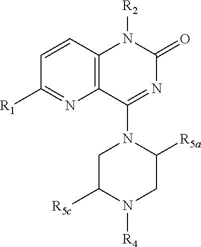

R₁ is —CN;
R₂ is —CH₃;
R₅ₐ is —CH₃ or —CH₂CH₃; and
R_{5c} is —CH₃, —CH₂CH₃, or —CH₂CH₂CH₃.

In some of any embodiments, the inhibitor is a compound of Formula (II) or a pharmaceutically acceptable salt thereof having the structure:

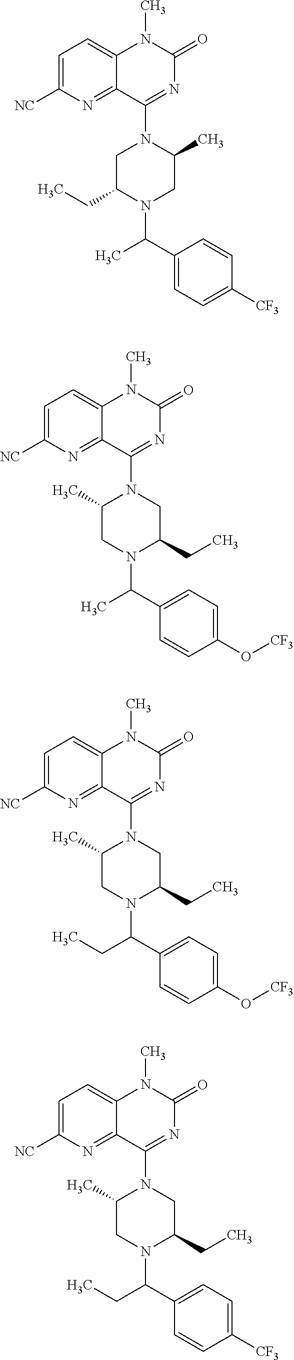 [SMILES: CC[C@@H]1CN(c2nc(=O)n(C)c3ccc(C#N)nc23)[C@@H](C)CN1C(C)c1ccc(C(F)(F)F)cc1 ; CC[C@@H]1CN(c2nc(=O)n(C)c3ccc(C#N)nc23)[C@@H](C)CN1C(C)c1ccc(OC(F)(F)F)cc1 ; CCC(c1ccc(OC(F)(F)F)cc1)N1C[C@H](C)N(c2nc(=O)n(C)c3ccc(C#N)nc23)C[C@H]1CC ; CCC(c1ccc(C(F)(F)F)cc1)N1C[C@H](C)N(c2nc(=O)n(C)c3ccc(C#N)nc23)C[C@H]1CC]

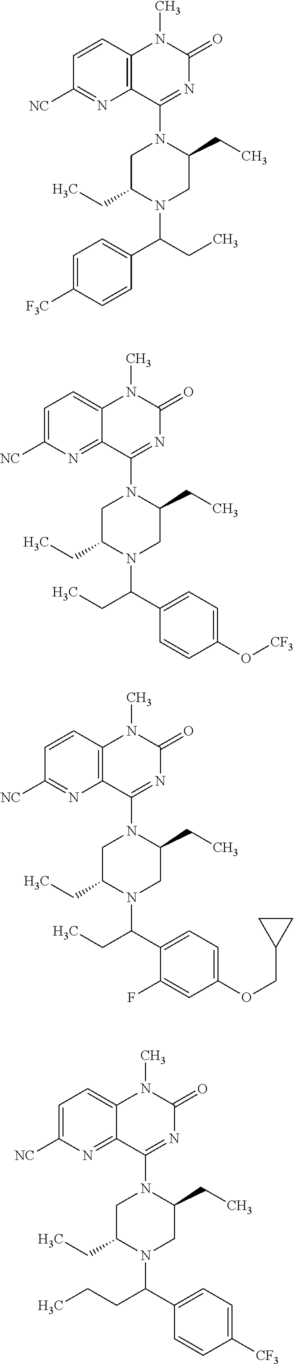 [SMILES: CCC(c1ccc(C(F)(F)F)cc1)N1C[C@H](CC)N(c2nc(=O)n(C)c3ccc(C#N)nc23)C[C@H]1CC ; CCC(c1ccc(OC(F)(F)F)cc1)N1C[C@H](CC)N(c2nc(=O)n(C)c3ccc(C#N)nc23)C[C@H]1CC ; CCC(c1ccc(OCC2CC2)cc1F)N1C[C@H](CC)N(c2nc(=O)n(C)c3ccc(C#N)nc23)C[C@H]1CC ; CCCC(c1ccc(C(F)(F)F)cc1)N1C[C@H](CC)N(c2nc(=O)n(C)c3ccc(C#N)nc23)C[C@H]1CC]

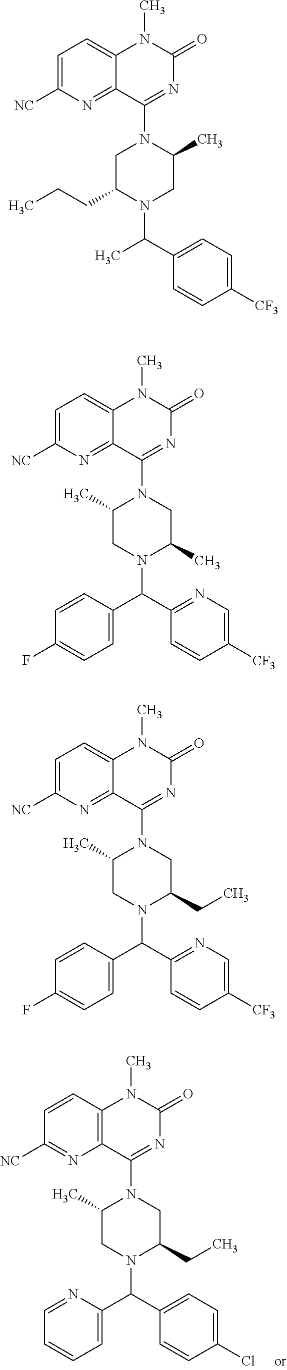 [SMILES: CCC[C@@H]1CN(c2nc(=O)n(C)c3ccc(C#N)nc23)[C@@H](C)CN1C(C)c1ccc(C(F)(F)F)cc1 ; C[C@@H]1CN(c2nc(=O)n(C)c3ccc(C#N)nc23)[C@@H](C)CN1C(c1ccc(F)cc1)c1ccc(C(F)(F)F)cn1 ; CC[C@@H]1CN(c2nc(=O)n(C)c3ccc(C#N)nc23)[C@@H](C)CN1C(c1ccc(F)cc1)c1ccc(C(F)(F)F)cn1 ; CC[C@@H]1CN(c2nc(=O)n(C)c3ccc(C#N)nc23)[C@@H](C)CN1C(c1ccc(Cl)cc1)c1ccccn1]

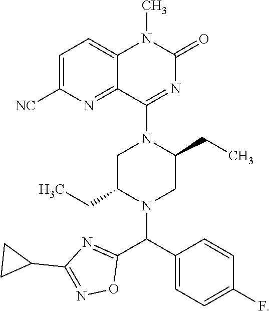 [SMILES: CC[C@H]1CN(C(c2ccc(F)cc2)c2nc(C3CC3)no2)[C@H](CC)CN1c1nc(=O)n(C)c2ccc(C#N)nc12]

In some of any embodiments, the inhibitor is a compound of Formula (II) or a pharmaceutically acceptable salt thereof having the structure:

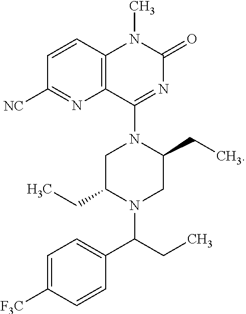 [SMILES: CCC(c1ccc(C(F)(F)F)cc1)N1C[C@H](CC)N(c2nc(=O)n(C)c3ccc(C#N)nc23)C[C@H]1CC]

In some of any embodiments, the inhibitor is administered in a therapeutically effective amount. In some of any embodiments, the therapeutically effective dose provides a dose of the inhibitor that is continuously therapeutically effective for the period of time that the inhibitor is administered.

In some of any embodiments, the inhibitor is administered in a per day amount between or between about 0.25 and 250 mg, inclusive. In some of any embodiments, the inhibitor is administered in a per day amount between or between about 0.5 and 100 mg, inclusive.

In some of any embodiments, the inhibitor is administered in a once per day amount between or between about 0.25 and 250 mg, inclusive. In some of any embodiments, the inhibitor is administered in a once per day amount between or between about 0.5 and 100 mg, inclusive.

In some of any embodiments, the inhibitor is administered orally.

In some of any embodiments, the recombinant receptor is an engineered T cell receptor (eTCR).

In some of any embodiments, the recombinant receptor is a chimeric antigen receptor (CAR).

In some of any embodiments, the antigen is CD19, and the CAR is an anti-CD19 CAR. In some of any embodiments, the CAR is the CAR of BREYANZI® (lisocabtagene maraleucel), TECARTUS™ (brexucabtagene autoleucel), KYMRIAH™ (tisagenlecleucel), or YESCARTA™ (axicabtagene ciloleucel). In some of any embodiments, the T cell therapy is BREYANZI® (lisocabtagene maraleucel), TECARTUS™ (brexucabtagene autoleucel), KYMRIAH™ (tisagenlecleucel), or YESCARTA™ (axicabtagene ciloleucel).

In some of any embodiments, the antigen is BCMA, and the CAR is an anti-BCMA CAR. In some of any embodiments, the CAR is the CAR of ABECMA® (idecabtagene vicleucel) or CARVYKTI™ (ciltacabtagene autoleucel). In some of any embodiments, the T cell therapy is ABECMA® (idecabtagene vicleucel) or CARVYKTI™ (ciltacabtagene autoleucel).

In some of any embodiments, the cancer is a solid tumor. In some of any embodiments, the cancer is a hematological (liquid) tumor.

In some of any embodiments, the cancer is a B cell malignancy.

In some of any embodiments, the cancer is a leukemia. In some of any embodiments, the cancer is a lymphoma. In some of any embodiments, the cancer is a myeloma. In some of any embodiments, the myeloma is multiple myeloma.

In some of any embodiments, the cancer is relapsed or refractory.

In some of any embodiments, the T cell therapy comprises between or between about 0.1×10⁶ and 1,000×10⁶ total recombinant receptor-expressing T cells, inclusive. In some of any embodiments, the T cell therapy comprises between or between about 10×10⁶ and 1,000×10⁶ total recombinant receptor-expressing T cells, inclusive. In some of any embodiments, the T cell therapy comprises between or between about 10×10⁶ and 500×10⁶ total recombinant receptor-expressing T cells, inclusive.

In some of any embodiments, the recombinant receptor-expressing T cells are viable recombinant receptor-expressing T cells.

In some of any embodiments, the T cell therapy is administered intravenously.

In some of any embodiments, the T cells of the T cell therapy are autologous to the subject. In some of any embodiments, the T cells of the T cell therapy are allogeneic to the subject.

In some of any embodiments, the T cells of the T cell therapy are human T cells.

Also provided herein in some embodiments is a T cell therapy comprising viable engineered T cells expressing a recombinant receptor directed against an antigen expressed by cells of a cancer for use as part of a combination therapy comprising the T cell therapy and an inhibitor of DGKα and/or DGKζ, wherein the T cell therapy and the inhibitor are administered to a subject having the cancer according to any of the described methods.

Also provided herein in some embodiments is an inhibitor of DGKα and/or DGKζ for use as part of a combination therapy comprising the inhibitor and a T cell therapy comprising viable engineered T cells expressing a recombinant receptor directed against an antigen expressed by cells of a cancer, wherein the T cell therapy and the inhibitor are administered to a subject having the cancer according to any of the described methods.

Also provided herein in some embodiments is a T cell therapy comprising viable engineered T cells expressing a recombinant receptor directed against an antigen expressed by cells of a cancer and an inhibitor of DGKα and/or DGKζ for use as part of a combination therapy comprising the T cell therapy and the inhibitor, wherein the T cell therapy and the inhibitor are administered to a subject having the cancer according to any of the described methods.

Also provided herein in some embodiments is use of a T cell therapy comprising viable engineered T cells expressing a recombinant receptor directed against an antigen expressed by cells of a cancer as part of a combination therapy comprising the T cell therapy and an inhibitor of DGKα and/or DGKζ, wherein the T cell therapy and the inhibitor are administered to a subject having the cancer according to any of the described methods.

Also provided herein in some embodiments is use of an inhibitor of DGKα and/or DGKζ as part of a combination therapy comprising the inhibitor and a T cell therapy comprising viable engineered T cells expressing a recombinant receptor directed against an antigen expressed by cells of a cancer, wherein the T cell therapy and the inhibitor are administered to a subject having the cancer according to any of the described methods.

Also provided herein in some embodiments is use of a T cell therapy comprising viable engineered T cells expressing a recombinant receptor directed against an antigen expressed by cells of a cancer and an inhibitor of DGKα and/or DGKζ as part of a combination therapy comprising the T cell therapy and the inhibitor, wherein the T cell therapy and the inhibitor are administered to a subject having the cancer according to any of the described methods.

Also provided herein in some embodiments is use of a T cell therapy comprising viable engineered T cells expressing a recombinant receptor directed against an antigen expressed by cells of a cancer in the manufacture of a medicament that is part of a combination therapy comprising the T cell therapy and an inhibitor of DGKα and/or DGKζ, wherein the T cell therapy and the inhibitor are administered to a subject having the cancer according to any of the described methods.

Also provided herein in some embodiments is use of an inhibitor of DGKα and/or DGKζ in the manufacture of a medicament that is part of a combination therapy comprising the inhibitor and a T cell therapy comprising viable engineered T cells expressing a recombinant receptor directed against an antigen expressed by cells of a cancer, wherein the T cell therapy and the inhibitor are administered to a subject having the cancer according to any of the described methods.

Also provided herein in some embodiments is use of a T cell therapy comprising viable engineered T cells expressing a recombinant receptor directed against an antigen expressed by cells of a cancer and an inhibitor of DGKα and/or DGKζ in the manufacture of a medicament that is a combination therapy comprising the T cell therapy and the inhibitor, wherein the T cell therapy and the inhibitor are administered to a subject having the cancer according to any of the described methods.

In some of any embodiments, the T cell therapy is any as described herein. In some embodiments, the inhibitor is any as described herein. In some embodiments, the subject is any as described herein. In some embodiments, the cancer is any as described herein.

### BRIEF DESCRIPTION OF DRAWINGS

FIGS. 1A and 1B show expression of activation and differentiation markers in CAR-expressing T cells following stimulation with a CAR-specific anti-idiotypic antibody in the presence of an exemplary DGK inhibitor (Compound 17). FIG. 1A shows the mean across donors of the log₂ fold change (presence vs. absence of Compound 17) in the % positive cells for the various activation and differentiation markers following stimulation with 3 μg/mL (left panel) or 30 μg/mL (right panel) of the CAR-specific anti-idiotypic antibody. FIG. 1B shows the mean across donors of the log 2 fold change (presence vs. absence of Compound 17) in MFI of cells following stimulation with 3 μg/mL (left panel) or 30 μg/mL (right panel) of the CAR-specific anti-idiotypic antibody. In FIGS. 1A and 1B, positive values are indicated with circles.

FIG. 2 shows growth curves (percent cellular confluence in well, as monitored using an Incucyte imaging system) of CAR-expressing cells stimulated with a CAR-specific anti-idiotypic antibody in the presence of Compound 17 or a control.

FIG. 3 shows the percent of specific lysis effected by CAR-expressing T cells co-cultured in the presence of Compound 17 (or a control) with CD19-antigen expressing target cells, K562.CD19 cells (high CD19 expression; left panel), Granta-519 cells (low CD19 expression; middle panel), and Raji cells (medium/high CD19 expression; right panel).

FIG. 4 shows the supernatant concentrations in pg/mL of IFNγ (left panel), IL-2 (middle panel), and TNFα (right panel) following co-culture of CAR-expressing T cells with K562.CD19 cells (circles), Granta-519 cells (squares), and Raji cells (triangles) in the presence of Compound 17.

FIGS. 5A-5B and 6A-6B show cytolytic activity and cytokine production during re-challenge of CAR-expressing T cells chronically stimulated in the presence of Compound 17 (concurrent treatment). FIG. 5A shows tumor cell numbers of K562.CD19 (left panel), Granta-519 (middle panel), and Raji cells (right panel) during 2D co-culture with CAR T cells that had been chronically stimulated in the presence of Compound 17. FIG. 5B shows the supernatant concentrations in pg/mL of IFNγ (left panel), IL-2 (middle panel), and TNFα (right panel) following 48 hours of 2D co-culture of CAR T cells with K562.CD19 cells (circles), Granta-519 cells (squares), and Raji cells (triangles). FIG. 6A shows normalized tumor volume of A549.CD19 (left panel) and Granta-519 tumor spheroids (right panel) at day 9 of co-culture with CAR T cells that had been concurrently treated with Compound 17 during chronic stimulation. FIG. 6B shows the supernatant concentrations in pg/mL of IFNγ (left panel), IL-2 (middle panel), and TNFα (right panel) following five days of 3D co-culture of CAR T cells with A549.CD19 (circles) and Granta-519 tumor spheroids (squares).

FIGS. 7A-7B and 8A-8B show cytolytic activity and cytokine production during re-challenge in the presence of Compound 17 of chronically stimulated CAR-expressing T cells (rescue treatment). FIG. 7A shows tumor cell numbers of K562.CD19 (left panel), Granta-519 (middle panel), and Raji cells (right panel) during 2D co-culture with CAR T cells in the presence of Compound 17. FIG. 7B shows the supernatant concentrations in pg/mL of IFNγ (left panel), IL-2 (middle panel), and TNFα (right panel) following 48 hours of 2D co-culture of CAR T cells with K562.CD19 cells (circles), Granta-519 cells (squares), and Raji cells (triangles) in the presence of Compound 17. FIG. 8A shows normalized tumor volume of A549.CD19 (left panel) and Granta-519 tumor spheroids (right panel) at day 9 of co-culture with CAR T cells in the presence of Compound 17. FIG. 8B shows the supernatant concentrations in pg/mL of IFNγ (left panel), IL-2 (middle panel), and TNFα (right panel) following five days of 3D co-culture of CAR T cells with A549.CD19 (circles) and Granta-519 tumor spheroids (squares) in the presence of Compound 17.

FIGS. 9 and 10 show cytolytic activity and proliferation of chronically stimulated T cells expressing an engineered T cell receptor (eTCR) following rescue treatment with Compound 17. FIG. 9 shows tumor cell numbers of CaSki cells monitored at various times during co-culture with chronically stimulated eTCR T cells in the presence of Compound 17 (left panel, eTCR T cells chronically stimulated with 10 μg/mL anti-Vbeta; right panel, eTCR T cells chronically stimulated with 20 μg/mL anti-Vbeta). FIG. 10 shows the cell counts for eTCR T cells for two donors, as shown from left to right: (1) chronically stimulated eTCR T cells with no compound treatment (control); (2) chronically stimulated eTCR T cells treated with Compound 17; or (3) fresh eTCR T cells that were not chronically stimulated.

FIG. 11 shows tumor burden in Raji cell-engrafted mice receiving three different dosing regimens (“Early”, “Delayed”, or “Continuous”) of the DGKi Compound 17 in combination with anti-CD19 CAR T cells. In FIG. 11, the shaded area indicates the period of time in which Compound 17 was administered orally once daily following CAR T cell administration in the Early, Delayed, or Continuous regimens.

FIG. 12 shows survival of the Raji cell-engrafted mice receiving the Early, Delayed, or Continuous Compound 17 regimen in combination with the anti-CD19 CAR T cells (n.s. [not significant]; *p<0.05; **p<0.01; ***p<0.001; ****p<0.0001).

FIG. 13 shows tumor control index values for the Raji cell-engrafted mice receiving the Early, Delayed, or Continuous Compound 17 regimen in combination with the anti-CD19 CAR T cells (n.s. [not significant]; *p<0.05; **p<0.01; ***p<0.001; ****p<0.0001).

FIG. 14 show tumor control index values with ANOVA results for tumor control index values for the Raji cell-engrafted mice receiving the Early, Delayed, or Continuous Compound 17 regimen in combination with the anti-CD19 CAR T cells (n.s. [not significant]; *p<0.05; **p<0.01; ***p<0.001; ****p<0.0001). In FIG. 14 and for ANOVA testing, results from all groups of vehicle-treated mice were pooled into one group for statistical comparison.

FIG. 15 shows the number of circulating CAR-expressing cells and the CD4:CD8 ratio of circulating T cells on Day 15 in the Raji cell-engrafted mice receiving the Delayed or Continuous regimen (n.s. [not significant]; *p<0.05; **p<0.01; ***p<0.001; ****p<0.0001).

FIG. 16 shows the total number of circulating CAR-expressing cells on Day 29 in the Raji cell-engrafted mice receiving the Early, Delayed, or Continuous Compound 17 regimen in combination with the anti-CD19 CAR T cells.

FIG. 17 shows tumor burden in Raji cell-engrafted mice receiving continuous dosing of the DGKi Compound 17 in combination with anti-CD19 CAR T cells. Compound 17 was administered orally once daily on Days 1-30 following CAR T cell administration on Day 1.

FIG. 18 shows survival of the Raji cell-engrafted mice receiving the continuous Compound 17 regimen in combination with the anti-CD19 CAR T cells.

FIG. 19 shows tumor control index values for the Raji cell-engrafted mice receiving the continuous Compound 17 regimen in combination with the anti-CD19 CAR T cells (n.s. [not significant]; *p<0.05; **p<0.01; ***p<0.001; ****p<0.0001).

FIG. 20 shows the number of circulating CAR-expressing cells following CAR T cell administration in the Raji cell-engrafted mice receiving the continuous regimen (n.s. [not significant]; *p<0.05; **p<0.01; ***p<0.001; ****p<0.0001).

FIG. 21 shows the number of circulating CAR-expressing cells from four healthy human donors following CAR T cell administration in the Raji cell-engrafted mice receiving DGKi administration on Days 1-7.

FIG. 22 shows tumor burden in Raji cell-engrafted mice receiving continuous dosing of the DGKi Compound 17 in combination with anti-CD19 CAR T cells or receiving DGKα/DGKζ (single or double knock-out anti-CD19 CAR T cells.

FIG. 23 shows survival of the Raji cell-engrafted mice receiving the continuous Compound 17 regimen in combination with the anti-CD19 CAR T cells or receiving DGKα/DGKζ single or double knock-out anti-CD19 CAR T cells (n.s. [not significant]; *p<0.05; **p<0.01; ***p<0.001; ****p<0.0001).

FIG. 24 shows tumor control index values for the Raji cell-engrafted mice receiving the continuous Compound 17 regimen in combination with the anti-CD19 CAR T cells or receiving DGKα/DGKζ (single or double knock-out anti-CD19 CAR T cells (n.s. [not significant]; *p<0.05; **p<0.01; ***p<0.001; ****p<0.0001).

FIG. 25 shows the number of circulating CAR-expressing cells following CAR T cell administration in the Raji cell-engrafted mice receiving the continuous regimen or in the Raji cell-engrafted mice receiving the DGKα/DGKζ single or double knock-out CAR T cells (n.s. [not significant]; *p<0.05; **p<0.01; ***p<0.001; ****p<0.0001).

FIG. 26A-26B show tumor burden in Nalm6 cell-engrafted mice. FIG. 26A shows tumor burden in Nalm6 cell-engrafted mice receiving continuous dosing of the DGKi Compound 17 in combination with anti-CD19 CAR T cells. FIG. 26B shows historical tumor burden data in Nalm6 cell-engrafted mice receiving anti-CD19 CAR T cells.

FIG. 27 shows survival of the Nalm6 cell-engrafted mice receiving the continuous Compound 17 regimen in combination with the anti-CD19 CAR T cells (n.s. [not significant]; * p<0.05; **p<0.01; ***p<0.001; ****p<0.0001).

FIG. 28 shows tumor control index values for the Nalm6 cell-engrafted mice receiving the continuous Compound 17 regimen in combination with the anti-CD19 CAR T cells (n.s. [not significant]; *p<0.05; **p<0.01; ***p<0.001; ****p<0.0001).

FIG. 29 shows the number of circulating CAR-expressing cells following CAR T cell administration in the Nalm6 cell-engrafted mice receiving the continuous regimen (n.s. [not significant]; *p<0.05; **p<0.01; ***p<0.001; ****p<0.0001).

FIG. 30 shows tumor burden following re-challenge of Nalm6 cell-engrafted mice that previously received continuous dosing of the DGKi Compound 17 in combination with anti-CD19 CAR T cells.

FIG. 31 shows survival following re-challenge of the Nalm6 cell-engrafted mice that previously received the continuous Compound 17 regimen in combination with the anti-CD19 CAR T cells (n.s. [not significant]; *p<0.05; **p<0.01; ***p<0.001; ****p<0.0001).

### DETAILED DESCRIPTION

Provided herein are combination therapies for use in treating a disease or condition involving administration of a T cell therapy directed against an antigen associated with the disease or condition, such as a chimeric antigen receptor (CAR)-T cell therapy or an engineered T cell receptor (eTCR)-T cell therapy, and a DGK inhibitor. In some embodiments, the T cell therapy is a composition including T cells for adoptive cell therapy in which the cells are engineered with a recombinant receptor targeting the antigen (e.g. a CAR or eTCR). In some embodiments, the disease or condition may be a cancer, infectious disease or autoimmune disease. In some embodiments, the DGKi is an inhibitor of DGKα, DGKζ, or both DGKα and DGKζ, such as a compound of Formula (I) or (II), such as a compound selected from compounds 1 to 34 or a pharmaceutically acceptable salt thereof. In some aspects, the provided methods and uses enhance or modulate proliferation and/or activity of T cells associated with administration of the T cell therapy (e.g. CAR-expressing T cells or eTCR-expressing T cells).

T cell-based therapies, such as adoptive T cell therapies (including those involving the administration of cells expressing recombinant antigen receptors specific for a disease or disorder of interest, such as CARs or eTCRs, can be effective in the treatment of cancer and other diseases and disorders. The engineered expression of recombinant receptors, such as CARs or eTCRs, on the surface of T cells enables the redirection of T-cell specificity. In clinical studies, CAR-T cells, for example anti-CD19 CAR-T cells, have produced durable, complete responses in both leukemia and lymphoma patients (Porter et al. (2015) Sci Transl Med., 7:303ra139; Kochenderfer (2015) J. Clin. Oncol., 33:540-9; Lee et al. (2015) Lancet, 385:517-28; Maude et al. (2014) N Engl J Med, 371:1507-17).

In certain contexts, available approaches to adoptive cell therapy may not always be entirely satisfactory. For example, in certain cases, although CAR T cell persistence can be detected in many subjects with lymphoma, fewer complete responses (CRs) have been observed in subjects with NHL compared to subjects with ALL. More specifically, while higher overall response rates of up to 80% (CR rate 47% to 60%) have been reported after CAR T cell infusion, responses in some are transient and subjects have been shown to relapse in the presence of persistent CAR T cells (Neelapu, 58th Annual Meeting of the American Society of Hematology (ASH): 2016; San Diego, CA, USA. Abstract No. LBA-6.2016; Abramson, Blood. 2016 Dec. 1; 128(22): 4192). Another study reported a long term CR rate of 40% (Schuster, Ann Hematol. 2016 October; 95(11): 1805-10).

In some aspects, an explanation for this is the immunological exhaustion of circulating T cells of the T cell therapy, e.g., CAR-expressing T cells, and/or changes in T lymphocyte populations. In some contexts, optimal efficacy can depend on the ability of the administered cells to recognize and bind to a target, e.g., target antigen, to traffic, localize to and successfully enter appropriate sites within the subject, tumors, and environments thereof. In some contexts, optimal efficacy can depend on the ability of the administered cells to become activated, expand, to exert various effector functions, including cytotoxic killing and secretion of various factors such as cytokines, to persist, including long-term, to differentiate, transition or engage in reprogramming into certain phenotypic states (such as long-lived memory, less-differentiated, and effector states), to avoid or reduce immunosuppressive conditions in the local microenvironment of a disease, to provide effective and robust recall responses following clearance and re-exposure to target ligand or antigen, and avoid or reduce exhaustion, anergy, peripheral tolerance, terminal differentiation, and/or differentiation into a suppressive state.

In some embodiments, the exposure and persistence of engineered cells of a T cell therapy is reduced or declines after administration to the subject. Yet, observations indicate that, in some cases, increased exposure of the subject to administered cells expressing the recombinant receptors (e.g., increased number of cells or duration over time) may improve efficacy and therapeutic outcomes in adoptive cell therapy. Preliminary analysis conducted following the administration of different CD19-targeting CAR-expressing T cells to subjects with various CD19-expressing cancers in multiple clinical trials revealed a correlation between greater and/or longer degree of exposure to the CAR-expressing cells and treatment outcomes. Such outcomes included patient survival and remission, even in individuals with severe or significant tumor burden.

In some embodiments, following long-term stimulation or exposure to antigen and/or exposure under conditions in the tumor microenvironment, T cells can over time become hypofunctional and/or exhibit features associated with exhausted state. In some aspects, this reduces the persistence and efficacy of the T cells against antigen and limits their ability to be effective. There is a need for methods to improve the efficacy and function of T cells of a T cell therapy, e.g., CAR-expressing T cells or eTCR-expressing T cells, particularly to minimize, reduce, prevent or reverse hypofunctional or exhaustive states.

Diacylglycerol kinases (DGKs) are lipid kinases that mediate the conversion of diacylglycerol to phosphatidic acid thereby terminating T cell functions propagated through the TCR signaling pathway. Thus, DGKs serve as intracellular checkpoints and inhibition of DGKs are expected to enhance T cell signaling pathways and T cell activation. Supporting evidence include knock-out mouse models of either DGKα or DGKζ which show a hyper-responsive T cell phenotype and improved anti-tumor immune activity (Riese M. J. et al., Journal of Biological Chemistry, (2011) 7:5254-5265; Zha Y et al., Nature Immunology, (2006) 12:1343; Olenchock B. A. et al., (2006) 11:1174-81). Furthermore, tumor infiltrating lymphocytes isolated from human renal cell carcinoma patients were observed to overexpress DGKα which resulted in inhibited T cell function (Prinz, P. U. et al., J Immunology (2012) 12:5990-6000). Thus, DGKα and DGKζ are viewed as targets for cancer immunotherapy (Riese M. J. et al., Front Cell Dev Biol. (2016) 4:108; Chen, S. S. et al., Front Cell Dev Biol. (2016) 4:130; Avila-Flores, A. et al., Immunology and Cell Biology (2017) 95:549-563; Noessner, E., Front Cell Dev Biol. (2017) 5:16; Krishna, S., et al., Front Immunology (2013) 4:178; Jing, W. et al., Cancer Research (2017) 77:5676-5686.

The provided methods are based on observations that a DGKi, such as the exemplary Compound 17 as described, improves T cell function, including functions related to the ability to produce one or more cytokines, cytotoxicity, expansion, proliferation, and persistence of T cells. In some aspects, the provided methods enhance or modulate proliferation and/or activity of T cell activity associated with administration of the T cell therapy (e.g. CAR-expressing T cells). It is found that such methods and uses provide for or achieve improved or greater T cell functionality, and thereby improved anti-tumor efficacy.

It also is found herein that, in addition to potentiating T cell function, such DGKi, e.g., Compound 17, exhibit effects to reverse, delay, or prevent T cell exhaustion, including by increasing T cell signaling and/or altering one or more genes that are differentially regulated following chronic (long-term) stimulation. Thus, while in some cases agents that increase or potentiate T cell activity may drive the cells to an exhausted state, it is found herein that activity of such DGKi, e.g., Compound 17, to exert a potentiating effect on T cell activity is decoupled from T cell exhaustion. In some embodiments, the provided methods involving compound administration of such DGKi, e.g., Compound 17, is capable of potentiating activity of T cells and delaying, limiting, reducing, inhibiting or preventing exhaustion.

Moreover, observations herein show that the DGKi, e.g., Compound 17, exhibits activity to rescue T cells from T cell exhaustion, such as by restoring or partially restoring one or more T cell activities after a cell has shown features of exhaustion. Remarkably, results herein show that exposure of T cells, that have been chronically stimulated and exhibit features of exhausted T cells, to a DGKi described herein, such as Compound 17, are able to recover activity or have their activity restored or partially restored. The observations herein support that the provided methods may also achieve improved or more durable responses as compared to certain alternative methods, such as in particular groups of subjects treated.

These observations were made using a chronic stimulation assay to render T cells (e.g. CAR T cells) hypofunctional (e.g. reduced cytolysis and IL-2 secretion). Using this model, engineered T cells (e.g. CAR T cells or eTCR T cells) were examined to assess impact of DGKi, such as Compound 17, on function of the engineered T cells when present during (concurrent) or following (rescue) exposure to conditions leading to a hypofunctional, exhaustive state. Upon rechallenge with antigen, the findings provided herein demonstrate that concurrent treatment of engineered T cells (e.g. CAR T cells or eTCR T cells) during such conditions reversed activity and phenotypes associated with T cell hypofunctionality and preserved more effector function. Likewise, the results show that the later exposure of the DGKi, e.g., Compound 17, could rescue or restore T cell function, including cytokine production and cytolytic activity, of exhausted T cells. Further, the results were seen with different recombinant antigen receptors including and CARs and eTCRs.

It is also shown herein that administration of the DGKi, e.g., Compound 17, in a continuous dosing scheme, e.g., at least once daily for a plurality of consecutive days, such as for 28 consecutive days, improves in vivo anti-tumor effects following administration of engineered T cells for the T cell therapy, e.g., CAR T cells or eTCR T cells. As demonstrated herein, the continuous presence of the DGKi following T cell therapy administration, such as beginning on the same day as T cell therapy administration, can improve CAR T cell expansion and/or persistence, in particular that of CD4+ CAR T cells. Without wishing to be limited by a specific mechanism of action, the results shown herein are consistent with a finding that the continuous presence of the DGKi following T cell therapy administration may prevent or reduce exhaustion in T cells of the T cell therapy, thereby improving T cell anti-tumor activity and proliferative capacity following administration.

The provided findings indicate that combination therapy of a DGKi that is an inhibitor of DGKα, DGKζ, or both DGKα and DGKζ, such as a compound of Formula (I) or (II), such as a compound selected from compounds 1 to 34 or a pharmaceutically acceptable salt thereof, in methods involving a T cell therapy, such as involving administration of a composition of engineered T cells, achieves improved function of the T cell therapy, such as by potentiating T cell activity and reducing, preventing or delaying T cell exhaustion or rescuing cells from T cell exhaustion. In some embodiments, the combination therapy involves administration or use of a T cell therapy (e.g., CAR T cells or eTCR T cells) and a DGKi that is a compound of Formula (II). In some embodiments, the combination therapy involves administration or use of a T cell therapy (e.g., CAR T cells or eTCR T cells) and a DGKi that is a compound that is 4-((2S,5R)-2,5-diethyl-4-(1-(4-(trifluoromethyl)phenyl)propyl)piperazin-1-yl)-1-methyl-2-oxo-1,2-dihydropyrido[3,2-d]pyrimidine-6-carbonitrile or a stereoisomer thereof. In some embodiments, the combination therapy involves administration or use of a T cell therapy (e.g., CAR T cells or eTCR T cells) and a DGKi that is a compound that is 4-((2S,5R)-2,5-diethyl-4-((S)-1-(4-(trifluoromethyl)phenyl)propyl)piperazin-1-yl)-1-methyl-2-oxo-1,2-dihydropyrido[3,2-d]pyrimidine-6-carbonitrile (Compound 17). In some embodiments, combination of the T cell therapy (e.g., administration of engineered T cells) with the DGKi, e.g., Compound 17, improves or enhances one or more functions and/or effects of the T cell therapy, such as persistence, expansion, cytotoxicity, and/or therapeutic outcomes, e.g., ability to kill or reduce the burden of tumor or other disease or target cell.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. COMBINATION THERAPY

Provided herein in some embodiments are methods of treatment that include administering a cell therapy and an inhibitor of DGK to a subject having a disease or condition. In some embodiments, the inhibitor of DGK is an inhibitor of DGKα and/or DGKζ. In some embodiments, the cell therapy is a T cell therapy. In some embodiments, the T cell therapy includes engineered T cells. In some embodiments, the engineered T cells express a recombinant receptor. In some embodiments, the recombinant receptor is a chimeric antigen receptor (CAR). In some embodiments, the recombinant receptor is an engineered T cell receptor (TCR).

In some embodiments, the DGK inhibitor, e.g., the inhibitor of DGKα and/or DGKζ, is administered prior to the T cell therapy, e.g., the initiation of administration of the inhibitor is carried out, is performed, or occurs prior to initiation of administration of the T cell therapy. In some embodiments, the DGK inhibitor, e.g., the inhibitor of DGKα and/or DGKζ, is administered concurrently with the T cell therapy, e.g., the initiation of administration of the inhibitor is carried out, is performed, or occurs concurrently with administration of the T cell therapy. In some embodiments, the DGK inhibitor, e.g., the inhibitor of DGKα and/or DGKζ, is administered after the T cell therapy, e.g., the initiation of administration of the inhibitor is carried out, is performed, or occurs after initiation of administration of the T cell therapy.

Also provided herein in some embodiments are methods of treatment that include administering an inhibitor of DGK to a subject having a disease or condition. Also provided herein in some embodiments are methods of rescuing engineered cells of a cell therapy from exhaustion, e.g., rescuing engineered T cells of a T cell therapy from exhaustion, said methods including administering an inhibitor of DGK to a subject having a disease or condition. Also provided herein in some embodiments are methods of reducing or delaying the onset of T cell exhaustion of T cells of a T cell therapy, said methods including administering an inhibitor of DGK to a subject having a disease or condition. In some embodiments, the subject has previously been administered a cell therapy, e.g., T cell therapy, for treatment of the disease or condition.

The combination therapy, e.g., including engineered cells expressing a recombinant receptor, such as a chimeric antigen receptor (CAR), and the DGK inhibitor, or compositions including the engineered cells and/or the DGK inhibitor described herein, are useful in a variety of therapeutic, diagnostic, and prophylactic indications. For example, the combinations are useful in treating a variety of diseases and disorders in a subject. Such methods and uses include therapeutic methods and uses, for example involving administration of the engineered cells, the DGK inhibitor, and/or compositions containing one or both to a subject having a disease, condition, or disorder, such as a tumor or cancer. In some embodiments, the engineered cells, the DGK inhibitor, and/or compositions containing one or both are administered in an effective amount to effect treatment of the disease or disorder. Uses include uses of the engineered cells, the DGK inhibitor, and/or compositions containing one or both in such methods and treatments, and in the preparation of a medicament in order to carry out such therapeutic methods. In some embodiments, the methods are carried out by administering the engineered cells, the DGK inhibitor, and/or compositions containing one or both to the subject having or suspected of having the disease or condition. In some embodiments, the methods thereby treat the disease, condition, or disorder in the subject. In some embodiments, the DGK inhibitor is any as described in Section I-B. In some embodiments, the engineered cells are any as described in Sections I and II.

The disease or condition that is treated can be any in which expression of an antigen is associated with and/or involved in the etiology of the disease or condition, e.g., antigen expression causes, exacerbates, or otherwise is involved in such disease or condition. Exemplary diseases and conditions include diseases or conditions associated with malignancy or transformation of cells (e.g., cancer), autoimmune or inflammatory disease, or an infectious disease, e.g., caused by a bacterial, viral, or other pathogen. Exemplary antigens, which include antigens associated with various diseases and conditions that can be treated, are described herein. In particular embodiments, the antigen-binding domain of the recombinant receptor, e.g., CAR or TCR, expressed by the cell therapy, e.g., T cell therapy, administered as part of the methods provided herein, specifically binds to an antigen associated with the disease or condition.

In some embodiments, the disease or condition includes tumors, including solid tumors, hematologic malignancies, and melanomas, and including localized and metastatic tumors, infectious diseases, such as infection with a virus or other pathogen, e.g., HIV, HCV, HBV, CMV, HPV, and parasitic disease, and autoimmune and inflammatory diseases. In some embodiments, the disease or condition is a tumor, cancer, malignancy, neoplasm, or other proliferative disease or disorder. Such diseases include but are not limited to leukemia, lymphoma, e.g., acute myeloid (or myelogenous) leukemia (AML), chronic myeloid (or myelogenous) leukemia (CML), acute lymphocytic (or lymphoblastic) leukemia (ALL), chronic lymphocytic leukemia (CLL), hairy cell leukemia (HCL), small lymphocytic lymphoma (SLL), Mantle cell lymphoma (MCL), Marginal zone lymphoma, Burkitt lymphoma, Hodgkin lymphoma (HL), non-Hodgkin lymphoma (NHL), Anaplastic large cell lymphoma (ALCL), follicular lymphoma, refractory follicular lymphoma, diffuse large B-cell lymphoma (DLBCL) and multiple myeloma (MM).

In some embodiments, the disease or condition is a B cell malignancy. In some embodiments, the B cell malignancy is selected from among acute lymphoblastic leukemia (ALL), adult ALL, chronic lymphoblastic leukemia (CLL), non-Hodgkin lymphoma (NHL), and Diffuse Large B-Cell Lymphoma (DLBCL). In some embodiments, the disease or condition is NHL. In some embodiments, the NHL is selected from the group consisting of aggressive NHL, diffuse large B cell lymphoma (DLBCL), NOS (de novo and transformed from indolent), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL), optionally, follicular lymphoma Grade 3B (FL3B).

In some embodiments, the disease or disorder is a B cell-related disorder. In some of any of the provided embodiments of the provided methods, the disease or disorder is an autoimmune disease or disorder. In some of any of the provided embodiments of the provided methods, the autoimmune disease or disorder is systemic lupus erythematosus (SLE), lupus nephritis, inflammatory bowel disease, rheumatoid arthritis, ANCA associated vasculitis, idiopathic thrombocytopenia purpura (ITP), thrombotic thrombocytopenia purpura (TTP), autoimmune thrombocytopenia, Chagas' disease, Grave's disease, Wegener's granulomatosis, poly-arteritis nodosa, Sjogren's syndrome, pemphigus vulgaris, scleroderma, multiple sclerosis, psoriasis, IgA nephropathy, IgM polyneuropathies, vasculitis, diabetes mellitus, Reynaud's syndrome, anti-phospholipid syndrome, Goodpasture's disease, Kawasaki disease, autoimmune hemolytic anemia, myasthenia gravis, or progressive glomerulonephritis.

In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

In some embodiments, the disease or disorder is a solid tumor, or a cancer associated with a non-hematological tumor. In some embodiments, the disease or disorder is a solid tumor, or a cancer associated with a solid tumor. In some embodiments, the disease or disorder is a pancreatic cancer, bladder cancer, colorectal cancer, breast cancer, prostate cancer, renal cancer, hepatocellular cancer, lung cancer, ovarian cancer, cervical cancer, pancreatic cancer, rectal cancer, thyroid cancer, uterine cancer, gastric cancer, esophageal cancer, head and neck cancer, melanoma, neuroendocrine cancers, CNS cancers, brain tumors, bone cancer, or soft tissue sarcoma. In some embodiments, the disease or disorder is a bladder, lung, brain, melanoma (e.g. small-cell lung, melanoma), breast, cervical, ovarian, colorectal, pancreatic, endometrial, esophageal, kidney, liver, prostate, skin, thyroid, or uterine cancers. In some embodiments, the disease or disorder is a pancreatic cancer, bladder cancer, colorectal cancer, breast cancer, prostate cancer, renal cancer, hepatocellular cancer, lung cancer, ovarian cancer, cervical cancer, pancreatic cancer, rectal cancer, thyroid cancer, uterine cancer, gastric cancer, esophageal cancer, head and neck cancer, melanoma, neuroendocrine cancers, CNS cancers, brain tumors, bone cancer, or soft tissue sarcoma.

In some embodiments, the antigen associated with the disease or condition is or includes αvβ6 integrin (avb6 integrin), B cell activating factor receptor (BAFF-R), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD70, CD123, CD133, CD138, CD171, chondroitin sulfate proteoglycan 4 (CSPG4), delta-like ligand 3 (DLL3), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.

In some embodiments, the disease or condition is a B cell malignancy. In some embodiments, the disease or condition is large B cell lymphoma. In some embodiments, the B cell malignancy is selected from among acute lymphoblastic leukemia (ALL), adult ALL, chronic lymphoblastic leukemia (CLL), non-Hodgkin lymphoma (NHL), and Diffuse Large B-Cell Lymphoma (DLBCL). In some embodiments, the disease or condition is NHL. In some embodiments, the NHL is selected from the group consisting of aggressive NHL, diffuse large B cell lymphoma (DLBCL), NOS (de novo and transformed from indolent), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL), optionally, follicular lymphoma Grade 3B (FL3B). In any of such embodiments, the disease or condition has relapsed or is refractory to one or more previous treatments (relapsed or refractory disease; R/R). In some aspects, the recombinant receptor, e.g., CAR or TCR, specifically binds to an antigen associated with the disease or condition or expressed in cells of the environment of a lesion associated with the B cell malignancy. Antigens targeted by the receptors in some embodiments include antigens associated with a B cell malignancy, such as any of a number of known B cell marker. In some embodiments, the antigen targeted by the receptor is BAFF-R, CD20, CD19, CD22, ROR1, CD45, CD21, CD5, CD33, Igkappa, Iglambda, CD79a, CD79b or CD30, or combinations thereof. For instance, the antigen in some embodiments is BAFF-R, and the CAR is as described in Qin et al., Science Translational Medicine 11 (511): eaaw9414 (2019). In some embodiments, the antigen targeted by the receptor is CD19. For instance, the recombinant receptor may be a CAR that is that of axicabtagene ciloleucel (Yescarta), tisagenlecleucel (Kymriah), or lisocabtagene maraleucel (Breyanzi). In some embodiments, the recombinant receptor may be a CAR that is that of TECARTUS™ (brexucabtagene autoleucel). In some embodiments, the T cell therapy, e.g., anti-CD19 CAR T cell therapy, is axicabtagene ciloleucel (Yescarta), tisagenlecleucel (Kymriah), lisocabtagene maraleucel (Breyanzi), or TECARTUS™ (brexucabtagene autoleucel).

In some embodiments, the disease or condition is a large B-cell lymphoma. In some embodiments, the large B-cell lymphoma is a relapsed or refractory large B-cell lymphoma. In some embodiments, the large B-cell lymphoma is a diffuse large B cell lymphoma (DLBCL) not otherwise specified (including DLBCL arising from indolent lymphoma), a high-grade B-cell lymphoma, a primary mediastinal large B-cell lymphoma, or a follicular lymphoma grade 3B. In some embodiments, the target antigen bound by the recombinant receptor and associated with the large B-cell lymphoma is CD19. In some embodiments, the recombinant receptor is an anti-CD19 CAR. In some embodiments, the anti-CD19 CAR is that of lisocabtagene maraleucel (Breyanzi). In some embodiments, the T cell therapy is an anti-CD19 CAR T cell therapy. In some embodiments, the anti-CD19 CAR T cell therapy is lisocabtagene maraleucel (Breyanzi, see Sehgal et al., 2020, Journal of Clinical Oncology 38:15_suppl, 8040; Teoh et al., 2019, Blood 134(Supplement_1):593; and Abramson et al., 2020, The Lancet 396(10254): 839-852).

In some embodiments, the disease or condition is mantle cell lymphoma or B-cell precursor acute lymphoblastic leukemia (ALL). In some embodiments, the disease or condition is relapsed or refractory mantle cell lymphoma or relapsed or refractory B-cell precursor acute lymphoblastic leukemia (ALL). In some embodiments, the target antigen bound by the recombinant receptor and associated with the disease or condition is CD19. In some embodiments, the recombinant receptor is an anti-CD19 CAR. In some embodiments, the anti-CD19 CAR is that of TECARTUS™ (brexucabtagene autoleucel). In some embodiments, the T cell therapy is an anti-CD19 CAR T cell therapy. In some embodiments, the anti-CD19 CAR T cell therapy is TECARTUS™ (brexucabtagene autoleucel, see Mian and Hill, 2021, Expert Opin Biol Ther; 21(4): 435-441; and Wang et al., 2021, Blood 138(Supplement 1):744).

In some embodiments, the disease or condition is diffuse large B-cell lymphoma (DLBCL) or acute lymphoblastic leukemia (ALL). In some embodiments, the disease or condition is relapsed or refractory diffuse large B-cell lymphoma (DLBCL) or relapsed or refractory acute lymphoblastic leukemia (ALL). In some embodiments, the target antigen bound by the recombinant receptor and associated with the disease or condition is CD19. In some embodiments, the recombinant receptor is an anti-CD19 CAR. In some embodiments, the anti-CD19 CAR is that of tisagenlecleucel (Kymriah). In some embodiments, the T cell therapy is an anti-CD19 CAR T cell therapy. In some embodiments, the anti-CD19 CAR T cell therapy is tisagenlecleucel (Kymriah, see Bishop et al., 2022, N Engl J Med 386:629:639; Schuster et al., 2019, N Engl J Med 380:45-56; Halford et al., 2021, Ann Pharmacother 55(4):466-479; Mueller et al., 2021, Blood Adv. 5(23):4980-4991; and Fowler et al., 2022, Nature Medicine 28:325-332).

In some embodiments, the disease or condition is a B-cell lymphoma. In some embodiments, the B-cell lymphoma is a relapsed or refractory B-cell lymphoma. In some embodiments, the B-cell lymphoma is diffuse large B-cell lymphoma (DLBCL), primary mediastinal B-cell lymphoma, high grade B-cell lymphoma, DLBCL that results from follicular lymphoma, or follicular lymphoma. In some embodiments, the target antigen bound by the recombinant receptor and associated with the B-cell lymphoma is CD19. In some embodiments, the recombinant receptor is an anti-CD19 CAR. In some embodiments, the anti-CD19 CAR is that of axicabtagene ciloleucel (Yescarta). In some embodiments, the T cell therapy is an anti-CD19 CAR T cell therapy. In some embodiments, the anti-CD19 CAR T cell therapy is axicabtagene ciloleucel (Yescarta, see Neelapu et al., 2017, N Engl J Med 377(26):2531-2544; Jacobson et al., 2021, The Lancet 23(1):P91-103; and Locke et al., 2022, N Engl J Med 386:640-654).

In some embodiments, the disease or condition is a myeloma, such as a multiple myeloma. Antigens targeted by the recombinant receptors in some embodiments include antigens associated with multiple myeloma. In some aspects, the antigen is expressed on multiple myeloma, such as B cell maturation antigen (BCMA), G protein-coupled receptor class C group 5 member D (GPRC5D), CD38 (cyclic ADP ribose hydrolase), CD138 (syndecan-1, syndecan, SYN-1), CS-1 (CS1, CD2 subset 1, CRACC, SLAMF7, CD319, and 19A24), BAFF-R, TACI and/or FcRH5. Other exemplary multiple myeloma antigens include CD56, TIM-3, CD33, CD123, CD44, CD20, CD40, CD74, CD200, EGFR, β2-Microglobulin, HM1.24, IGF-1R, IL-6R, TRAIL-R1, and the activin receptor type IIA (ActRIIA). See Benson and Byrd, J. Clin. Oncol. (2012) 30(16): 2013-15; Tao and Anderson, Bone Marrow Research (2011):924058; Chu et al., Leukemia (2013) 28(4):917-27; Garfall et al., Discov Med. (2014) 17(91):37-46. In some embodiments, the antigens include those present on lymphoid tumors, myeloma, AIDS-associated lymphoma, and/or post-transplant lymphoproliferations, such as CD38. Antibodies or antigen-binding fragments directed against such antigens are known and include, for example, those described in U.S. Pat. Nos. 8,153,765; 8,603,477, 8,008,450; U.S. Pub. No. US20120189622 or US20100260748; and/or International PCT Publication Nos. WO2006099875, WO2009080829 or WO2012092612 or WO2014210064. In some embodiments, such antibodies or antigen-binding fragments thereof (e.g. scFv) are contained in multispecific antibodies, multispecific chimeric receptors, such as multispecific CARs, and/or multispecific cells.

In some embodiments, the disease or disorder is a multiple myeloma (MM). In some embodiments, the disease or disorder is associated with expression of G protein-coupled receptor class C group 5 member D (GPRC5D) and/or expression of B cell maturation antigen (BCMA). In some embodiments, the subject has or is suspected of having a MM that is associated with expression of a tumor-associated antigen, such as a B cell maturation antigen (BCMA), G protein-coupled receptor class C group 5 member D (GPRC5D), or Fc receptor like 5 (FCRL5; also known as Fc receptor homolog 5 or FCRH5). In some aspects, the recombinant receptor, e.g., CAR or TCR, specifically binds to an antigen associated with the MM, such as specifically binds to BCMA, GPRC5D, or FCRL5. In some embodiments, the target antigen associated with the disease or condition, e.g., with the MM, is BCMA. In some embodiments, the recombinant receptor is the CAR, e.g., anti-BCMA CAR, of ABECMA® (idecabtagene vicleucel) or CARVYKTI™ (ciltacabtagene autoleucel). In some embodiments, the T cell therapy, e.g., anti-BCMA CAR T cell therapy, is ABECMA® (idecabtagene vicleucel) or CARVYKTI™ (ciltacabtagene autoleucel).

In some embodiments, the disease or condition is a multiple myeloma. In some embodiments, the multiple myeloma is a relapsed or refractory multiple myeloma. In some embodiments, the target antigen bound by the recombinant receptor and associated with the multiple myeloma is BCMA. In some embodiments, the recombinant receptor is an anti-BCMA CAR. In some embodiments, the anti-BCMA CAR is that of CARVYKTI™ (ciltacabtagene autoleucel). In some embodiments, the T cell therapy is an anti-BCMA CAR T cell therapy. In some embodiments, the anti-BCMA CAR T cell therapy is CARVYKTI™ (ciltacabtagene autoleucel, see Berdeja et al., Lancet. 2021 Jul. 24; 398(10297):314-324; and Martin, Abstract #549 [Oral], presented at 2021 American Society of Hematology (ASH) Annual Meeting & Exposition)).

In some embodiments, the disease or condition is a multiple myeloma. In some embodiments, the multiple myeloma is a relapsed or refractory multiple myeloma. In some embodiments, the target antigen bound by the recombinant receptor and associated with the multiple myeloma is BCMA. In some embodiments, the recombinant receptor is an anti-BCMA CAR. In some embodiments, the anti-BCMA CAR is that of ABECMA® (idecabtagene vicleucel). In some embodiments, the T cell therapy is an anti-BCMA CAR T cell therapy. In some embodiments, the anti-BCMA CAR T cell therapy is ABECMA® (idecabtagene vicleucel, see Raje et al., 2019, N Engl J Med 380:1726-1737; and Munshi et al., 2021, N Engl J Med 384:705-716).

In some embodiments, the disease or disorder is a chronic lymphocytic leukemia (CLL). In some embodiments, the subject has or is suspected of having a CLL that is associated with expression of a tumor-associated antigen, such as a Receptor Tyrosine Kinase Like Orphan Receptor 1 (ROR1). In some embodiments, the antigen is ROR1, and the disease or disorder is CLL. In some aspects, the recombinant receptor, e.g., CAR or TCR, specifically binds to an antigen associated with the CLL, such as specifically binds to ROR1.

In some embodiments, the disease or disorder is a non-small cell lung cancer (NSCLC). In some embodiments, the subject has or is suspected of having a NSCLC that is associated with expression of a tumor-associated antigen, such as a Receptor Tyrosine Kinase Like Orphan Receptor 1 (ROR1). In some embodiments, the antigen is ROR1, and the disease or disorder is NSCLC. In some aspects, the recombinant receptor, e.g., CAR or TCR, specifically binds to an antigen associated with the NSCLC, such as specifically binds to ROR1. In some embodiments, the CAR is as described in Specht et al., Cancer Res 79:4 Supplement, Abstract P2-09-13.

In some embodiments, the disease or disorder is a triple negative breast cancer (TNBC). In some embodiments, the subject has or is suspected of having a TNBC that is associated with expression of a tumor-associated antigen, such as a Receptor Tyrosine Kinase Like Orphan Receptor 1 (ROR1). In some embodiments, the antigen is ROR1, and the disease or disorder is TNBC. In some aspects, the recombinant receptor, e.g., CAR or TCR, specifically binds to an antigen associated with the TNBC, such as specifically binds to ROR1. In some embodiments, the CAR is as described in Specht et al., Cancer Res 79:4 Supplement, Abstract P2-09-13.

In some embodiments, the disease or disorder is a small cell lung cancer (SCLC), optionally a relapsed/refractory SCLC. In some embodiments, the subject has or is suspected of having a SCLC that is associated with expression of a tumor-associated antigen, such as DLL3. In some embodiments, the antigen is DLL3, and the disease or disorder is SCLC. In some aspects, the recombinant receptor, e.g., CAR or TCR, specifically binds to an antigen associated with the SCLC, such as specifically binds to DLL3. In some embodiments, the CAR is as described in Byers et al., Journal of Clinical Oncology 37, no. 15_suppl (2019).

In some embodiments, the disease or disorder is a renal cell carcinoma (RCC). In some embodiments, the subject has or is suspected of having an RCC that is associated with expression of a tumor-associated antigen, such as CD70. In some embodiments, the antigen is CD70, and the disease or disorder is RCC. In some aspects, the recombinant receptor, e.g., CAR or TCR, specifically binds to an antigen associated with the RCC, such as specifically binds to CD70. In some embodiments, the CAR is as described in Panowski et al., Cancer Res 79 (13 Supplement) 2326 (2019).

In some embodiments, the disease or disorder is an acute myeloid leukemia (AML). In some embodiments, the subject has or is suspected of having an AML that is associated with expression of a tumor-associated antigen, such as CD70. In some embodiments, the antigen is CD70, and the disease or disorder is AML. In some aspects, the recombinant receptor, e.g., CAR or TCR, specifically binds to an antigen associated with the AML, such as specifically binds to CD70. In some embodiments, the CAR is as described in Sauer et al., Blood 134 (Supplement_1): 1932 (2019).

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

### A. Administration of a T Cell Therapy

In some aspects, the methods provided herein include combination therapy by administering a cell therapy, e.g., T cell therapy, to a subject having a disease or condition, such as any disease or condition as described above, in combination with a DGK inhibitor. In some aspects, the methods provided herein include administering a DGK inhibitor to a subject in combination with (e.g., prior to, concurrently with, or subsequent to) administration of the cell therapy, e.g., T cell therapy. In some embodiments, provided methods include administering a DGK inhibitor to a subject that has previously been administered a cell therapy, e.g., T cell therapy, for treatment of a disease or condition. In some embodiments, the cell therapy, e.g., T cell therapy, is administered to the subject prior to administering the DGKi to the subject. In some embodiments, the cell therapy, e.g., T cell therapy, and the DGKi are administered concurrently.

In some embodiments, the cell therapy is a T cell therapy. The T cell therapy may include T cells engineered with a recombinant receptor, such as a CAR or an engineered TCR (e.g., eTCR). In some embodiments, the T cell therapy includes T cells (e.g., CD4+ and/or CD8+ T cells) that express a CAR that targets or binds an antigen associated with the disease or condition. In some embodiments, the T cell therapy includes T cells (e.g., CD4+ and/or CD8+ T cells) that express an eTCR that targets or binds an antigen associated with the disease or condition. A T cell therapy for use in the provided methods include any of a variety of CAR-expressing or eTCR-expressing T cells. It is within the level of a skilled artisan to choose the appropriate recombinant receptor (e.g., CAR or eTCR)-expressing T cell therapy for treating the disease or condition. Examples of T cell therapies, including CAR-expressing T cells and eTCR-expressing T cells, are described in Section II. In some embodiments, the T cells of the T cell therapy are allogenic to the subject being treated. In some embodiments, the T cells of the T cell therapy are autologous to the subject being treated.

Methods for administration of cells for adoptive cell therapy are known and may be used in connection with the provided methods, compositions and articles of manufacture and kits. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells of the T cell therapy can be administered in a composition formulated for administration, or alternatively, in more than one composition (e.g., two compositions) formulated for separate administration. The dose(s) of the cells may include a particular number or relative number of cells or of the engineered cells, and/or a defined ratio or compositions of two or more sub-types within the composition, such as CD4 vs CD8 T cells.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells. In some embodiments, administration of the cell dose or any additional therapies, e.g., the lymphodepleting therapy, intervention therapy and/or combination therapy, is carried out via outpatient delivery.

For the treatment of disease, the appropriate dosage may depend on the type of disease to be treated, the type of cells or recombinant receptors, the severity and course of the disease, previous therapy, the subject's clinical history and response to the cells, and the discretion of the attending physician. The compositions and cells are in some embodiments suitably administered to the subject at one time or over a series of treatments.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702(2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In some embodiments, the dose of cells of the T cell therapy, such a T cell therapy comprising cells engineered with a recombinant antigen receptor, e.g. CAR or TCR, is provided as a composition or formulation, such as a pharmaceutical composition or formulation. Such compositions can be used in accord with the provided methods, such as in the prevention or treatment of diseases, conditions, and disorders.

In some embodiments, the T cell therapy, such as engineered T cells (e.g. CAR or TCR T cells), are formulated with a pharmaceutically acceptable carrier. In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being prevented or treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

The pharmaceutical composition in some embodiments contains cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The cells may be administered using standard administration techniques, formulations, and/or devices. Provided are formulations and devices, such as syringes and vials, for storage and administration of the compositions. With respect to cells, administration can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

In some cases, the cell therapy is administered as a single pharmaceutical composition comprising the cells. In some embodiments, a given dose is administered by a single bolus administration of the cells or agent. In some embodiments, it is administered by multiple bolus administrations of the cells or agent, for example, over a period of no more than 3 days, or by continuous infusion administration of the cells or agent.

In some embodiments, a dose of cells is administered to subjects in accord with the provided combination therapy methods. In some embodiments, the size or timing of the doses is determined as a function of the particular disease or condition in the subject. One may empirically determine the size or timing of the doses for a particular disease in view of the provided description.

In certain embodiments, the cells, or individual populations of sub-types of cells, are administered to the subject at a range of about 0.1 million to about 100 billion cells and/or that amount of cells per kilogram of body weight of the subject, such as, e.g., 0.1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells), about 10 million to about 600 million cells, about 100 million to about 600 million cells, about 100 million to about 450 million cells, about 150 million cells to about 300 or 450 million cells, or any value in between these ranges and/or per kilogram of body weight of the subject. Dosages may vary depending on attributes particular to the disease or disorder and/or patient and/or other treatments. In some embodiments, such values refer to numbers of recombinant receptor-expressing cells (e.g., CAR- or TCR-expressing cells); in other embodiments, they refer to number of T cells or PBMCs or total cells administered.

In some embodiments, the cell therapy comprises administration of a dose comprising a number of cell from or from about 1×10⁵ to 1×10⁸ total recombinant receptor-expressing cells, total T cells, or total peripheral blood mononuclear cells (PBMCs), from or from about 5×10⁵ to 1×10⁷ total recombinant receptor-expressing cells, total T cells, or total peripheral blood mononuclear cells (PBMCs) or from or from about 1×10⁶ to 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or about at least 1×10⁵ total recombinant receptor-expressing cells, total T cells, or total peripheral blood mononuclear cells (PBMCs), such at least or at least 1×10⁶, at least or about at least 1×10⁷, at least or about at least 1×10⁸ of such cells.

In some embodiments, for example, where the subject is a human, the dose includes fewer than about 5×10⁸ total recombinant receptor (e.g., TCR or CAR)-expressing cells, T cells, or peripheral blood mononuclear cells (PBMCs), e.g., in the range of about 1×10⁶ to 5×10⁸ such cells, such as 2×10⁶, 5×10⁶, 1×10⁷, 5×10⁷, 1×10⁸, or 5×10⁸ total such cells, or the range between any two of the foregoing values.

In some embodiments, the number is with reference to the total number of CD3+ or CD8+, in some cases also recombinant receptor-expressing (e.g. TCR+ or CAR+) cells. In some embodiments, the cell therapy comprises administration of a dose comprising a number of cell from or from about 1×10⁵ to 1×10⁸ CD3+ or CD8+ total T cells or CD3+ or CD8+ recombinant receptor-expressing cells, from or from about 5×10⁵ to 1×10⁷ CD3+ or CD8+ total T cells or CD3+ or CD8+ recombinant receptor-expressing cells, or from or from about 1×10⁶ to 1×10⁷ CD3+ or CD8+ total T cells or CD3+ or CD8+ recombinant receptor-expressing cells, each inclusive. In some embodiments, the cell therapy comprises administration of a dose comprising a number of cell from or from about 1×10⁵ to 1×10⁸ total CD3+/CAR+ or CD8+/CAR+ cells, from or from about 5×10⁵ to 1×10⁷ total CD3+/CAR+ or CD8+/CAR+ cells, or from or from about 1×10⁶ to 1×10⁷ total CD3+/CAR+ or CD8+/CAR+ cells, each inclusive.

In some embodiments, the dose of genetically engineered cells comprises from or from about 1×10⁵ to 5×10⁸ total recombinant receptor-expressing T cells, 1×10⁵ to 2.5×10⁸ total recombinant receptor-expressing T cells, 1×10⁵ to 1×10⁸ total recombinant receptor-expressing T cells, 1×10⁵ to 5×10⁷ total recombinant receptor-expressing T cells, 1×10⁵ to 2.5×10⁷ total recombinant receptor-expressing T cells, 1×10⁵ to 1×10⁷ total recombinant receptor-expressing T cells, 1×10⁵ to 5×10⁶ total recombinant receptor-expressing T cells, 1×10⁵ to 2.5×10⁶ total recombinant receptor-expressing T cells, 1×10⁵ to 1×10⁶ total recombinant receptor-expressing T cells, 1×10⁶ to 5×10⁸ total recombinant receptor-expressing T cells, 1×10⁶ to 2.5×10⁸ total recombinant receptor-expressing T cells, 1×10⁶ to 1×10⁸ total recombinant receptor-expressing T cells, 1×10⁶ to 5×10⁷ total recombinant receptor-expressing T cells, 1×10⁶ to 2.5×10⁷ total recombinant receptor-expressing T cells, 1×10⁶ to 1×10⁷ total recombinant receptor-expressing T cells, 1×10⁶ to 5×10⁶ total recombinant receptor-expressing T cells, 1×10⁶ to 2.5×10⁶ total recombinant receptor-expressing T cells, 2.5×10⁶ to 5×10⁸ total recombinant receptor-expressing T cells, 2.5×10⁶ to 2.5×10⁸ total recombinant receptor-expressing T cells, 2.5×10⁶ to 1×10⁸ total recombinant receptor-expressing T cells, 2.5×10⁶ to 5×10⁷ total recombinant receptor-expressing T cells, 2.5×10⁶ to 2.5×10⁷ total recombinant receptor-expressing T cells, 2.5×10⁶ to 1×10⁷ total recombinant receptor-expressing T cells, 2.5×10⁶ to 5×10⁶ total recombinant receptor-expressing T cells, 5×10⁶ to 5×10⁸ total recombinant receptor-expressing T cells, 5×10⁶ to 2.5×10⁸ total recombinant receptor-expressing T cells, 5×10⁶ to 1×10⁸ total recombinant receptor-expressing T cells, 5×10⁶ to 5×10⁷ total recombinant receptor-expressing T cells, 5×10⁶ to 2.5×10⁷ total recombinant receptor-expressing T cells, 5×10⁶ to 1×10⁷ total recombinant receptor-expressing T cells, 1×10⁷ to 5×10⁸ total recombinant receptor-expressing T cells, 1×10⁷ to 2.5×10⁸ total recombinant receptor-expressing T cells, 1×10⁷ to 1×10⁸ total recombinant receptor-expressing T cells, 1×10⁷ to 5×10⁷ total recombinant receptor-expressing T cells, 1×10⁷ to 2.5×10⁷ total recombinant receptor-expressing T cells, 2.5×10⁷ to 5×10⁸ total recombinant receptor-expressing T cells, 2.5×10⁷ to 2.5×10⁸ total recombinant receptor-expressing T cells, 2.5×10⁷ to 1×10⁸ total recombinant receptor-expressing T cells, 2.5×10⁷ to 5×10⁷ total recombinant receptor-expressing T cells, 5×10⁷ to 5×10⁸ total recombinant receptor-expressing T cells, 5×10⁷ to 2.5×10⁸ total recombinant receptor-expressing T cells, 5×10⁷ to 1×10⁸ total recombinant receptor-expressing T cells, 1×10⁸ to 5×10⁸ total recombinant receptor-expressing T cells, 1×10⁸ to 2.5×10⁸ total recombinant receptor-expressing T cells, or 2.5×10⁸ to 5×10⁸ total recombinant receptor-expressing T cells.

In some embodiments, the dose of genetically engineered cells comprises at least or at least about 1×10⁵ TCR- or CAR-expressing cells, at least or at least about 2.5×10⁵ TCR- or CAR-expressing cells, at least or at least about 5×10⁵ TCR- or CAR-expressing cells, at least or at least about 1×10⁶ TCR- or CAR-expressing cells, at least or at least about 2.5×10⁶ TCR- or CAR-expressing cells, at least or at least about 5×10⁶ TCR- or CAR-expressing cells, at least or at least about 1×10⁷ TCR- or CAR-expressing cells, at least or at least about 2.5×10⁷ TCR- or CAR-expressing cells, at least or at least about 5×10⁷ TCR- or CAR-expressing cells, at least or at least about 1×10⁸ TCR- or CAR-expressing cells, at least or at least about 2.5×10⁸ TCR- or CAR-expressing cells, or at least or at least about 5×10⁸ TCR- or CAR-expressing cells.

In some embodiments, the dose of genetically engineered cells comprises from or from about 15 million to 1 billion total recombinant receptor-expressing T cells. In some embodiments, the dose of genetically engineered cells comprises from or from about 15 million to 600 million total recombinant receptor-expressing T cells. In some embodiments, the dose of genetically engineered cells comprises from or from about 150 million to 600 million total recombinant receptor-expressing T cells. In some embodiments, the dose of genetically engineered cells comprises from or from about 150 million to 450 million total recombinant receptor-expressing T cells.

In some embodiments, the dose of genetically engineered cells comprises at least or at least about 1×10⁵ recombinant receptor-expressing cells, at least or at least about 2.5×10⁵ recombinant receptor-expressing cells, at least or at least about 5×10⁵ recombinant receptor-expressing cells, at least or at least about 1×10⁶ recombinant receptor-expressing cells, at least or at least about 2.5×10⁶ recombinant receptor-expressing cells, at least or at least about 5×10⁶ recombinant receptor-expressing cells, at least or at least about 1×10⁷ recombinant receptor-expressing cells, at least or at least about 2.5×10⁷ recombinant receptor-expressing cells, at least or at least about 5×10⁷ recombinant receptor-expressing cells, at least or at least about 1×10⁸ recombinant receptor-expressing cells, at least or at least about 2.5×10⁸ recombinant receptor-expressing cells, or at least or at least about 5×10⁸ recombinant receptor-expressing cells.

In some embodiments, the cell therapy comprises administration of a dose comprising a number of cell from or from about 1×10⁵ to 1×10⁸ total recombinant receptor-expressing cells, total T cells, or total peripheral blood mononuclear cells (PBMCs), from or from about 5×10⁵ to 1×10⁷ total recombinant receptor-expressing cells, total T cells, or total peripheral blood mononuclear cells (PBMCs) or from or from about 1×10⁶ to 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or about at least 1×10⁵ total recombinant receptor-expressing cells, total T cells, or total peripheral blood mononuclear cells (PBMCs), such at least or at least 1×10⁶, at least or about at least 1×10⁷, at least or about at least 1×10⁸ of such cells.

In some embodiments, for example, where the subject is a human, the dose includes fewer than about 5×10⁸ total recombinant receptor (e.g., CAR or TCR)-expressing cells, T cells, or peripheral blood mononuclear cells (PBMCs), e.g., in the range of about 1×10⁶ to 5×10⁸ such cells, such as 2×10⁶, 5×10⁶, 1×10⁷, 5×10⁷, 1×10⁸, or 5×10⁸ total such cells, or the range between any two of the foregoing values.

In some embodiments, the number is with reference to the total number of CD3+ or CD8+, in some cases also recombinant receptor-expressing (e.g. CAR or TCR expressing) cells. In some embodiments, the cell therapy comprises administration of a dose comprising a number of cell from or from about 1×10⁵ to 1×10⁸ CD3+ or CD8+ total T cells or CD3+ or CD8+ recombinant receptor-expressing cells, from or from about 5×10⁵ to 1×10⁷ CD3+ or CD8+ total T cells or CD3+ or CD8+ recombinant receptor-expressing cells, or from or from about 1×10⁶ to 1×10⁷ CD3+ or CD8+ total T cells or CD3+ or CD8+ recombinant receptor-expressing cells, each inclusive. In some embodiments, the cell therapy comprises administration of a dose comprising a number of cell from or from about 1×10⁵ to 1×10⁸ total CD3+/recombinant receptor+ or CD8+/recombinant receptor+ cells, from or from about 5×10⁵ to 1×10⁷ total CD3+/recombinant receptor+ or CD8+/recombinant receptor+ cells, or from or from about 1×10⁶ to 1×10⁷ total CD3+/recombinant receptor+ or CD8+/recombinant receptor+ cells, each inclusive.

In some embodiments, the cell therapy comprises administration of a dose comprising a number of cell from or from about 1×10⁵ to 5×10⁸ total recombinant receptor-expressing cells, total T cells, or total peripheral blood mononuclear cells (PBMCs), from or from about 5×10⁵ to 1×10⁷ total recombinant receptor-expressing cells, total T cells, or total peripheral blood mononuclear cells (PBMCs) or from or from about 1×10⁶ to 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or at least about 1×10⁵ total recombinant receptor-expressing cells, total T cells, or total peripheral blood mononuclear cells (PBMCs), such at least or at least 1×10⁶, at least or at least about 1×10⁷, at least or at least about 1×10⁸ of such cells. In some embodiments, the number is with reference to the total number of CD3+ or CD8+, in some cases also recombinant receptor-expressing (e.g. CAR+ or TCR+) cells. In some embodiments, the cell therapy comprises administration of a dose comprising a number of cell from or from about 1×10⁵ to 5×10⁸ CD3+ or CD8+ total T cells or CD3+ or CD8+ recombinant receptor-expressing cells, from or from about 5×10⁵ to 1×10⁷ CD3+ or CD8+ total T cells or CD3+ or CD8+ recombinant receptor-expressing cells, or from or from about 1×10⁶ to 1×10⁷ CD3+ or CD8+ total T cells or CD3+ or CD8+ recombinant receptor-expressing cells, each inclusive. In some embodiments, the cell therapy comprises administration of a dose comprising a number of cell from or from about 1×10⁵ to 5×10⁸ total CD3+/recombinant receptor+ or CD8+/recombinant receptor+ cells, from or from about 5×10⁵ to 1×10⁷ total CD3+/recombinant receptor+ or CD8+/recombinant receptor+ cells, or from or from about 1×10⁶ to 1×10⁷ total CD3+/recombinant receptor+ or CD8+/recombinant receptor+ cells, each inclusive.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between about 1×10⁶ and 5×10⁸ total recombinant receptor (e.g., CAR or TCR)-expressing CD8+ cells, e.g., in the range of about 5×10⁶ to 1×10⁸ such cells, such cells 1×10⁷, 2.5×10⁷, 5×10⁷, 7.5×10⁷, 1×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to 0.75×10⁸ total recombinant receptor-expressing CD8+ T cells, 1×10⁷ to 2.5×10⁷ total recombinant receptor-expressing CD8+ T cells, from or from about 1×10⁷ to 0.75×10⁸ total recombinant receptor-expressing CD8+ T cells, each inclusive. In some embodiments, the dose of cells comprises the administration of or about 1×10⁷, 2.5×10⁷, 5×10⁷7.5×10⁷, 1×10⁸, or 5×10⁸ total recombinant receptor-expressing CD8+ T cells.

In some embodiments, the dose of cells, e.g., recombinant receptor-expressing T cells, is administered to the subject as a single dose or is administered only one time within a period of two weeks, one month, three months, six months, 1 year or more.

In some embodiments, the cell therapy comprises administration of a dose comprising a number of cells that is at least or at least about or is or is about 0.1×10⁶ cells/kg body weight of the subject, 0.2×10⁶ cells/kg, 0.3×10⁶ cells/kg, 0.4×10⁶ cells/kg, 0.5×10⁶ cells/kg, 1×10⁶ cell/kg, 2.0×10⁶ cells/kg, 3×10⁶ cells/kg or 5×10⁶ cells/kg.

In some embodiments, the cell therapy comprises administration of a dose comprising a number of cells is between or between about 0.1×10⁶ cells/kg body weight of the subject and 1.0×10⁷ cells/kg, between or between about 0.5×10⁶ cells/kg and 5×10⁶ cells/kg, between or between about 0.5×10⁶ cells/kg and 3×10⁶ cells/kg, between or between about 0.5×10⁶ cells/kg and 2×10⁶ cells/kg, between or between about 0.5×10⁶ cells/kg and 1×10⁶ cell/kg, between or between about 1.0×10⁶ cells/kg body weight of the subject and 5×10⁶ cells/kg, between or between about 1.0×10⁶ cells/kg and 3×10⁶ cells/kg, between or between about 1.0×10⁶ cells/kg and 2×10⁶ cells/kg, between or between about 2.0×10⁶ cells/kg body weight of the subject and 5×10⁶ cells/kg, between or between about 2.0×10⁶ cells/kg and 3×10⁶ cells/kg, or between or between about 3.0×10⁶ cells/kg body weight of the subject and 5×10⁶ cells/kg, each inclusive.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g. antigen-expressing, such as CAR-expressing cells or TCR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, nor more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g. antigen-expressing, such as CAR-expressing cells or TCR expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In the context of adoptive cell therapy, administration of a given “dose” of cells encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, which is no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

The term “split dose” refers to a dose that is split so that it is administered over more than one day. This type of dosing is encompassed by the present methods and is considered to be a single dose. In some embodiments, the cells of a split dose are administered in a plurality of compositions, collectively comprising the cells of the dose, over a period of no more than three days.

Thus, the dose of cells may be administered as a split dose. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4+ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In particular embodiments, the numbers and/or concentrations of cells refer to the number of recombinant receptor (e.g., CAR or TCR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, administration of the DGK inhibitor in combination with the cells is able to significantly increase the expansion or proliferation of the cells, and thus a lower dose of cells can be administered to the subject. In some cases, the provided methods allow a lower dose of such cells to be administered, to achieve the same or better efficacy of treatment as the dose in a method in which the cell therapy is administered without administering the DGK inhibitor, such as at least 1.5-fold, 2-fold, 3-fold, 4-fold, 5-fold or 10-fold less than the dose in a method in which the cell therapy is administered without administering the DGK inhibitor.

In some embodiments, for example, the dose contains between or between about 5.0×10⁶ and 2.25×10⁷, 5.0×10⁶ and 2.0×10⁷, 5.0×10⁶ and 1.5×10⁷, 5.0×10⁶ and 1.0×10⁷, 5.0×10⁶ and 7.5×10⁶, 7.5×10⁶ and 2.25×10⁷, 7.5×10⁶ and 2.0×10⁷, 7.5×10⁶ and 1.5×10⁷, 7.5×10⁶ and 1.0×10⁷, 1.0×10⁷ and 2.25×10⁷, 1.0×10⁷ and 2.0×10⁷, 1.0×10⁷ and 1.5×10⁷, 1.5×10⁷ and 2.25×10⁷, 1.5×10⁷ and 2.0×10⁷, 2.0×10⁷ and 2.25×10⁷. In some embodiments, the dose of cells contains a number of cells, that is between at least or at least about 5×10⁶, 6×10⁶, 7×10⁶, 8×10⁶, 9×10⁶, 10×10⁶ and about 15×10⁶ recombinant-receptor expressing cells, such as recombinant-receptor expressing cells that are CD8+. In some embodiments, such dose, such as such target number of cells refers to the total recombinant-receptor expressing cells in the administered composition.

In some embodiments, for example, the lower dose contains less than about 5×10⁶ cells, recombinant receptor (e.g. TCR- or CAR)-expressing cells, T cells, and/or PBMCs per kilogram body weight of the subject, such as less than about 4.5×10⁶, 4×10⁶, 3.5×10⁶, 3×10⁶, 2.5×10⁶, 2×10⁶, 1.5×10⁶, 1×10⁶, 5×10⁵, 2.5×10⁵, or 1×10⁵ such cells per kilogram body weight of the subject. In some embodiments, the lower dose contains less than about 1×10⁵, 2×10⁵, 5×10⁵, or 1×10⁶ of such cells per kilogram body weight of the subject, or a value within the range between any two of the foregoing values. In some embodiments, such values refer to numbers of recombinant receptor-expressing cells; in other embodiments, they refer to number of T cells or PBMCs or total cells administered.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses. In some embodiments, one or more subsequent dose of cells can be administered to the subject. In some embodiments, the subsequent dose of cells is administered greater than or greater than about 7 days, 14 days, 21 days, 28 days, or 35 days after initiation of administration of the first dose of cells. The subsequent dose of cells can be more than, approximately the same as, or less than the first dose. In some embodiments, administration of the T cell therapy, such as administration of the first and/or second dose of cells, can be repeated.

In some embodiments, initiation of administration of the cell therapy, e.g. the dose of cells or a first dose of a split dose of cells, is administered before (prior to), concurrently with or after (subsequently or subsequent to) the administration of the inhibitor of DGKα and/or DGKζ. In some embodiments, initiation of administration of the cell therapy relative to initiation of administration of the inhibitor of DGKα and/or DGKζ is as described in any of the embodiments described in Section I-B-2.

In some embodiments, the method involves, subsequent to administering the dose of cells of the T cell therapy, e.g., adoptive T cell therapy, but prior to administering the DGK inhibitor, assessing a sample from the subject for one or more function of T cells, such as expansion or persistence of the cells, e.g. as determined by level or amount in the blood, or other phenotypes or desired outcomes as described herein, e.g., such as those described in Section III. In some embodiments, the method involves, subsequent to administering the dose of cells of the T cell therapy, e.g., adoptive T cell therapy, but prior to administering the DGK inhibitor, assessing a sample from the subject for expression of one or more exhaustion markers. Various parameters for determining or assessing the regimen of the combination therapy are described in Section III.

B. Administration of Dgkα and/or Dgkζ Inhibitors

In some aspects, the methods provided herein include a combination therapy by administering a DGK inhibitor in combination with a cell therapy, e.g., T cell therapy, to a subject having a disease or condition, such as any disease or condition as described above. The DGKi may be administered prior to, concurrently with, or subsequent to administration of a cell therapy, e.g. T cell therapy. In some aspects, the methods provided herein include administering a cell therapy, e.g., a T cell therapy, to a subject having a disease or condition, wherein the subject has previously been administered a DGK inhibitor. In some embodiments, the DGK inhibitor and cell therapy, e.g., T cell therapy, are administered to the subject concurrently.

In some embodiments, the inhibitor of DGK is an inhibitor of DGKα and/or DGKζ. In certain embodiments, an inhibitor of DGKα and/or DGKζ is an inhibitor of DGKα. In certain embodiments, an inhibitor of DGKα and/or DGKζ is an inhibitor of DGKζ. In certain embodiments, an inhibitor of DGKα and/or DGKζ inhibits both enzymes. The level of enzyme inhibition may be measured as further described herein. In certain embodiments, an inhibitor of DGKα and/or DGKζ is not a significant inhibitor of other DGK enzymes.

In certain embodiments, an inhibitor of DGKα and/or DGKζ increases an immune response, such as by increasing T cell activity. For example, an inhibitor of DGKα and/or DGKζ may increase primary T cell signaling, as evidenced, e.g., by an increase in pERK/pPKC signaling, which may be measured as further described herein. In certain embodiments, an inhibitor of DGKα and/or DGKζ has one or more of the following properties: (i) it lowers the threshold for antigen stimulation; (ii) increases CTL effector function; and (iii) enhances tumor cell killing. When an inhibitor of DGKα and/or DGKζ enhances tumor cell killing, this activity may be dependent on CD8+ T cells, as shown, e.g., in the CT26 animal model. When an inhibitor of DGKα and/or DGKζ enhances tumor cell killing, this activity may be dependent on NK cells, as shown, e.g., in the CT26 animal model. When an inhibitor of DGKα and/or DGKζ enhances tumor cell killing, this activity may be dependent on CD8+ T cells and NK cells, as shown, e.g., in the CT26 animal model. When an inhibitor of DGKα and/or DGKζ enhances tumor cell killing, this activity may be enhanced by CD4 cell depletion, e.g., in the CT-26 animal model. In certain embodiments, an inhibitor of DGKα and/or DGKζ enhances AH1+ Tetramer antigen presentation in the CT-26 animal model. An inhibitor of DGKα and/or DGKζ preferably includes one or more of the above cited properties, and may include all of them.

1. Exemplary Inhibitors of DGKα and/or DGKζ Enzyme Activity

Exemplary compounds, such as compounds of Formula I described herein and pharmaceutically acceptable salts thereof, are described in WO 2020/006018 and WO 2020/006016, the contents of both of which are specifically incorporated by reference herein. Exemplary compounds, such as compounds of Formula II described herein and pharmaceutically acceptable salts thereof, are described in PCT/US2020/048070, the contents of which are specifically incorporated by reference herein.

In some embodiments, the provided combination therapy of a T cell therapy and a DGK inhibitor involves administration or use of an inhibitor of DGKα and/or DGKζ that is a compound of Formula (I):

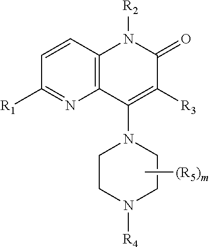

or a pharmaceutically acceptable salt thereof, wherein:
R₁ is H, F, Cl, Br, —CN, C₁₋₃ alkyl substituted with zero to 4 R₁ₐ, C₃₋₄ cycloalkyl substituted with zero to 4 R₁ₐ, C₁₋₃ alkoxy substituted with zero to 4 R₁ₐ, —NRₐRₐ, —S(O)ₙRₑ, or —P(O)RₑRₑ;
each R₁ₐ is independently F, Cl, —CN, —OH, —OCH₃, or —NRₐRₐ;
each Rₐ is independently H or C₁₋₃ alkyl;
each Rₑ is independently C₃₋₄ cycloalkyl or C₁₋₃ alkyl substituted with zero to 4 R₁ₐ;
R₂ is H, C₁₋₃ alkyl substituted with zero to 4 R₂ₐ, or C₃₋₄ cycloalkyl substituted with zero to 4 R₂ₐ;
each R₂ₐ is independently F, Cl, —CN, —OH, —O(C₁₋₂ alkyl), C₃₋₄ cycloalkyl, C₃₋₄ alkenyl, or C₃₋₄ alkynyl;
R₃ is H, F, Cl, Br, —CN, C₁₋₃ alkyl, C₁₋₂ fluoroalkyl, C₃₋₄ cycloalkyl, C₃₋₄ fluorocycloalkyl, or —NO₂;
R₄ is —CH₂R₄ₐ, —CH₂CH₂R₄ₐ, —CH₂CHR₄ₐR_{4d}, —CHR₄ₐR_{4b}, or —CR₄ₐR_{4b}R_{4c};
R₄ₐ and R_{4b} are independently:
(i) C₁₋₆ alkyl substituted with zero to 4 substituents independently selected from F, Cl, —CN, —OH, —OCH₃, —SCH₃, C₁₋₃ fluoroalkoxy, —NRₐRₐ, —S(O)₂Rₑ, or —NRₐS(O)₂Rₑ;
(ii) C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, C₁₋₄ hydroxyalkyl, —(CH₂)₁₋₂O(C₁₋₃ alkyl), C₁₋₄ alkoxy, —O(C₁₋₄ hydroxyalkyl), —O(CH)₁₋₃O(C₁₋₃ alkyl), C₁₋₃ fluoroalkoxy, —O(CH)₁₋₃NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₃ alkyl)₂, —S(O)₂(C₁₋₃ alkyl), —O(CH₂)₁₋₂(C₃₋₆ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or
(iii) C₁₋₄ alkyl substituted with one cyclic group selected from C₃₋₆ cycloalkyl, heterocyclyl, aryl, and heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl;
or R₄ₐ and R_{4b} together with the carbon atom to which they are attached form a C₃₋₆ cycloalkyl or a 3- to 6-membered heterocyclyl, each substituted with zero to 3 R_{f};
each R_{f} is independently F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, and —NR_{c}R_{c};
R_{4c} is C₁₋₆ alkyl or C₃₋₆ cycloalkyl, each substituted with zero to 4 substituents independently selected from F, Cl, —OH, C₁₋₂ alkoxy, C₁₋₂ fluoroalkoxy, and —CN;
R_{4d} is —OCH₃;
each R_{c} is independently H or C₁₋₂ alkyl;
R_{d} is phenyl substituted with zero to 1 substituent selected from F, Cl, —CN, —CH₃, and —OCH₃;
each R₅ is independently —CN, C₁₋₆ alkyl substituted with zero to 4 R_{g}, C₂₋₄ alkenyl substituted with zero to 4 R_{g}, C₂₋₄ alkynyl substituted with zero to 4 R_{g}, C₃₋₄ cycloalkyl substituted with zero to 4 R_{g}, phenyl substituted with zero to 4 R_{g}, oxadiazolyl substituted with zero to 3 R_{g}, pyridinyl substituted with zero to 4 R_{g}, —(CH₂)₁₋₂(heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each R_{g} is independently F, Cl, —CN, —OH, C₁₋₃ alkoxy, C₁₋₃ fluoroalkoxy, —O(CH₂)₁₋₂O(C₁₋₂ alkyl), or —NR_{c}R_{c};
m is zero, 1, 2, or 3; and
n is zero, 1, or 2.

In some embodiments, the provided combination therapy of a T cell therapy and a DGK inhibitor involves administration or use of an inhibitor of DGKα and/or DGKζ that is a compound of Formula (I) or a pharmaceutically acceptable salt thereof, wherein:
R₁ is H, F, Cl, Br, —CN, C₁₋₃ alkyl substituted with zero to 4 R₁ₐ, cyclopropyl substituted with zero to 3 R₁ₐ, C₁₋₃ alkoxy substituted with zero to 3 R₁ₐ, —NRₐRₐ, —S(O)ₙCH₃, or —P(O)(CH₃)₂;
each R₁ₐ is independently F, Cl, or —CN;
each Rₐ is independently H or C₁₋₃ alkyl;
R₂ is H or C₁₋₂ alkyl substituted with zero to 2 R₂ₐ;
each R₂ₐ is independently F, Cl, —CN, —OH, —O(C₁₋₂ alkyl), cyclopropyl, C₃₋₄ alkenyl, or C₃₋₄ alkynyl;
R₃ is H, F, Cl, Br, —CN, C₁₋₂ alkyl, —CF₃, cyclopropyl, or —NO₂;
R₄ₐ and R_{4b} are independently:
(i) C₁₋₄ alkyl substituted with zero to 4 substituents independently selected from F, Cl, —CN, —OH, —OCH₃, —SCH₃, C₁₋₃ fluoroalkoxy, and —NRₐRₐ;
(ii) C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, —CH₂OH, —(CH₂)₁₋₂O(C₁₋₂ alkyl), C₁₋₄ alkoxy, —O(C₁₋₄ hydroxyalkyl), —O(CH)₁₋₂O(C₁₋₂ alkyl), C₁₋₃ fluoroalkoxy, —O(CH)₁₋₂NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —O(CH₂)₁₋₂(C₃₋₄ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or
(iii) C₁₋₃ alkyl substituted with one cyclic group selected from C₃₋₆ cycloalkyl, heterocyclyl, phenyl, and heteroaryl, said cyclic group substituted with zero to 3 substituents independently selected from F, Cl, Br, —OH, —CN, C₁₋₃ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₄ cycloalkyl;
or R₄ₐ and R_{4b} together with the carbon atom to which they are attached, form a C₃₋₆ cycloalkyl or a 3- to 6-membered heterocyclyl, each substituted with zero to 3 R_{f};
each R_{f} is independently F, Cl, Br, —OH, —CN, C₁₋₄ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₄ alkyl, C₁₋₂ fluoroalkyl, C₁₋₃ alkoxy, C₁₋₂ fluoroalkoxy, and —NR_{c}R_{c};
R_{4c} is C₁₋₄ alkyl or C₃₋₆ cycloalkyl, each substituted with zero to 4 substituents independently selected from F, Cl, —OH, C₁₋₂ alkoxy, C₁₋₂ fluoroalkoxy, and —CN;
and each R₅ is independently —CN, C₁₋₅ alkyl substituted with zero to 4 R_{g}, C₂₋₃ alkenyl substituted with zero to 4 R_{g}, C₂₋₃ alkynyl substituted with zero to 4 R_{g}, C₃₋₄ cycloalkyl substituted with zero to 4 R_{g}, phenyl substituted with zero to 3 R_{g}, oxadiazolyl substituted with zero to 3 R_{g}, pyridinyl substituted with zero to 3 R_{g}, —(CH₂)₁₋₂(heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl).

In some embodiments, the provided combination therapy of a T cell therapy and a DGK inhibitor involves administration or use of an inhibitor of DGKα and/or DGKζ that is a compound of Formula (I) or a pharmaceutically acceptable salt thereof having the structure:

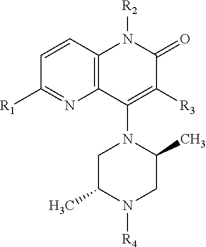

wherein:
R₁ is —CN;
R₂ is —CH₃;
R₃ is H, F, or —CN;
R₄ is:

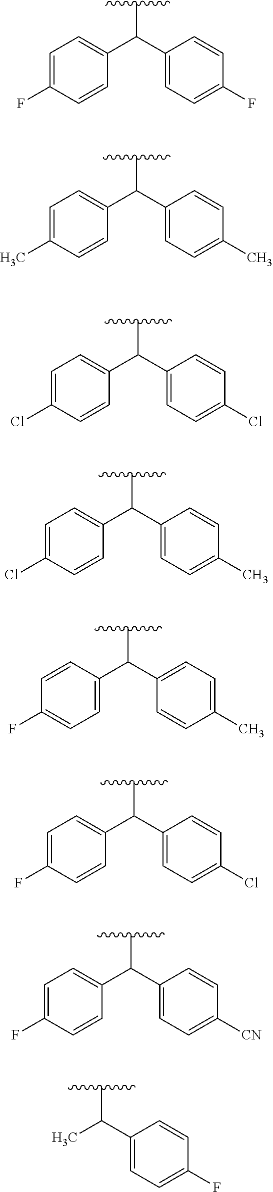

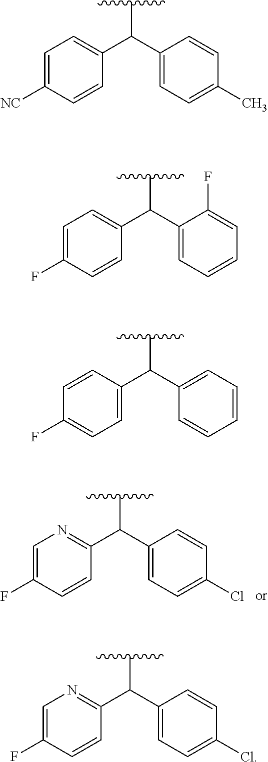 [SMILES: *C(c1ccc(C)cc1)c1ccc(C#N)cc1 ; *C(c1ccc(F)cc1)c1ccccc1F ; *C(c1ccccc1)c1ccc(F)cc1 ; *C(c1ccc(Cl)cc1)c1ccc(F)cn1 ; *C(c1ccc(Cl)cc1)c1ccc(F)cn1]

In some embodiments, the provided combination therapy of a T cell therapy and a DGK inhibitor involves administration or use of an inhibitor of DGKα and/or DGKζ that is a compound of Formula (I) or a pharmaceutically acceptable salt thereof having one the following structure or formula (or an isomer thereof):

### Methyl 1-(bis(4-fluorophenyl)methyl)-4-(6-cyano-1-methyl-2-oxo-1,2-dihydro-1,5-naphthyridin-4-yl)piperazine-2-carboxylate

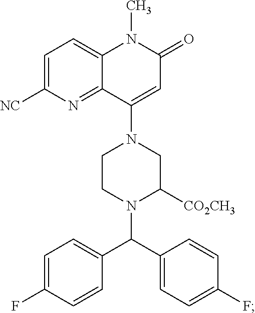 [SMILES: COC(=O)C1CN(c2cc(=O)n(C)c3ccc(C#N)nc23)CCN1C(c1ccc(F)cc1)c1ccc(F)cc1]

### 4-((2R,5S)-4-(bis(4-fluorophenyl)methyl)-2,5-dimethylpiperazin-1-yl)-6-bromo-1-methyl-2-oxo-1,2-dihydro-1,5-naphthyridine-3-carbonitrile

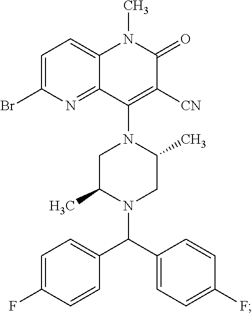 [SMILES: C[C@@H]1CN(C(c2ccc(F)cc2)c2ccc(F)cc2)[C@@H](C)CN1c1c(C#N)c(=O)n(C)c2ccc(Br)nc12]

### (R)-8-(4-(bis(4-fluorophenyl)methyl)-3-methylpiperazin-1-yl)-5-methyl-6-oxo-5,6-dihydro-1,5-naphthyridine-2,7-dicarbonitrile

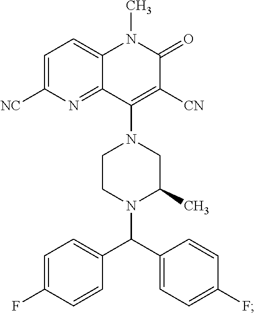 [SMILES: C[C@@H]1CN(c2c(C#N)c(=O)n(C)c3ccc(C#N)nc23)CCN1C(c1ccc(F)cc1)c1ccc(F)cc1]

### 8-[(2S,5R)-4-[(4-chlorophenyl)(5-methylpyridin-2-yl)methyl]-2,5-dimethylpiperazin-1-yl]-5-methyl-6-oxo-5,6-dihydro-1,5-naphthyridine-2-carbonitrile

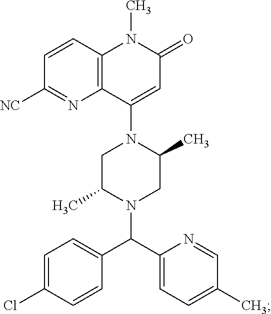 [SMILES: Cc1ccc(C(c2ccc(Cl)cc2)N2C[C@H](C)N(c3cc(=O)n(C)c4ccc(C#N)nc34)C[C@H]2C)nc1]

### 4-[(2S,5R)-4-[(4-chlorophenyl)(4-fluorophenyl)methyl]-2,5-dimethylpiperazin-1-yl]-6-methoxy-1-methyl-1,2-dihydro-1,5-naphthyridin-2-one

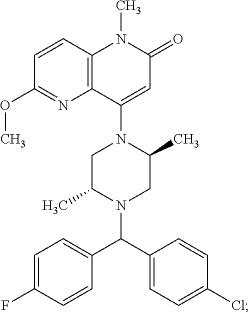 [SMILES: COc1ccc2c(n1)c(N1C[C@@H](C)N(C(c3ccc(F)cc3)c3ccc(Cl)cc3)C[C@@H]1C)cc(=O)n2C]

### 8-[(2S,5R)-4-{[2-(difluoromethyl)-4-fluorophenyl]methyl}-2,5-dimethylpiperazin-1-yl]-5-methyl-6-oxo-5,6-dihydro-1,5-naphthyridine-2-carbonitrile

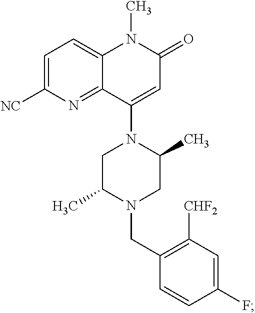 [SMILES: C[C@@H]1CN(c2cc(=O)n(C)c3ccc(C#N)nc23)[C@@H](C)CN1Cc1ccc(F)cc1C(F)F]

### 8-[(2S,5R)-4-[(4-fluorophenyl)(4-methylphenyl)methyl]-2,5-dimethylpiperazin-1-yl]-5-methyl-6-oxo-5,6-dihydro-1,5-naphthyridine-2-carbonitrile

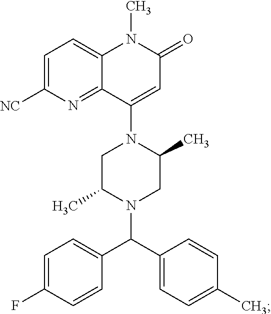 [SMILES: Cc1ccc(C(c2ccc(F)cc2)N2C[C@H](C)N(c3cc(=O)n(C)c4ccc(C#N)nc34)C[C@H]2C)cc1]

### 8-[(2S,5R)-4-[1-(2,6-difluorophenyl)ethyl]-2,5-dimethylpiperazin-1-yl]-5-methyl-6-oxo-5,6-dihydro-1,5-naphthyridine-2-carbonitrile

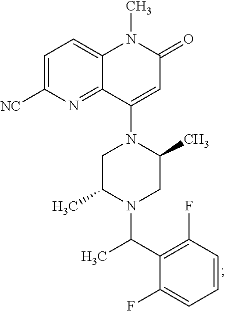 [SMILES: CC(c1c(F)cccc1F)N1C[C@H](C)N(c2cc(=O)n(C)c3ccc(C#N)nc23)C[C@H]1C]

### 8-((3R)-4-((4-chlorophenyl)(5-fluoropyridin-2-yl)methyl)-3-methylpiperazin-1-yl)-5-methyl-6-oxo-5,6-dihydro-1,5-naphthyridine-2,7-dicarbonitrile

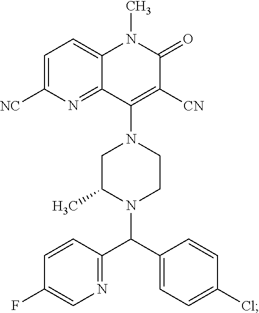 [SMILES: C[C@@H]1CN(c2c(C#N)c(=O)n(C)c3ccc(C#N)nc23)CCN1C(c1ccc(Cl)cc1)c1ccc(F)cn1]

### 8-(4-(bis(4-fluorophenyl)methyl)piperazin-1-yl)-5-methyl-7-nitro-6-oxo-5,6-dihydro-1,5-naphthyridine-2-carbonitrile

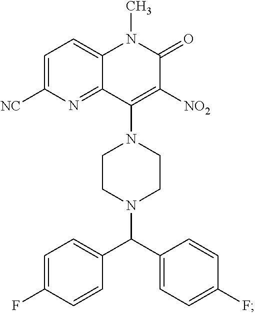 [SMILES: Cn1c(=O)c([N+](=O)[O-])c(N2CCN(C(c3ccc(F)cc3)c3ccc(F)cc3)CC2)c2nc(C#N)ccc21]

and

### 8-[(2S,5R)-4-[bis(4-methylphenyl)methyl]-2,5-dimethylpiperazin-1-yl]-5-methyl-6-oxo-5,6-dihydro-1,5-naphthyridine-2-carbonitrile

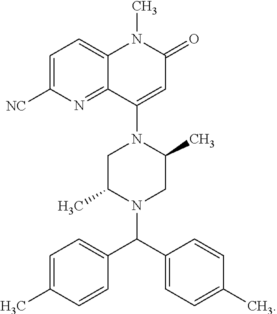 [SMILES: Cc1ccc(C(c2ccc(C)cc2)N2C[C@H](C)N(c3cc(=O)n(C)c4ccc(C#N)nc34)C[C@H]2C)cc1]

In some embodiments, the provided combination therapy of a T cell therapy and a DGK inhibitor involves administration or use of an inhibitor of DGKα and/or DGKζ that is a compound of Formula (II):

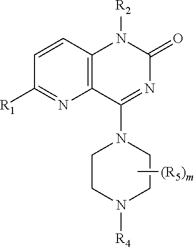

or a salt thereof, wherein:
R₁ is H, F, Cl, Br, —CN, —OH, C₁₋₃ alkyl substituted with zero to 4 R₁ₐ, C₃₋₄ cycloalkyl substituted with zero to 4 R₁ₐ, C₁₋₃ alkoxy substituted with zero to 4 R₁ₐ, —NRₐRₐ, —S(O)ₙRₑ, or —P(O)RₑRₑ;
each R₁ₐ is independently F, Cl, —CN, —OH, —OCH₃, or —NRₐRₐ;
each Rₐ is independently H or C₁₋₃ alkyl;
each Rₑ is independently C₃₋₄ cycloalkyl or C₁₋₃ alkyl substituted with zero to 4 R₁ₐ;
R₂ is H, C₁₋₃ alkyl substituted with zero to 4 R₂ₐ, or C₃₋₄ cycloalkyl substituted with zero to 4 R₂ₐ;
each R₂ₐ is independently F, Cl, —CN, —OH, —O(C₁₋₂ alkyl), C₃₋₄ cycloalkyl, C₃₋₄ alkenyl, or C₃₋₄ alkynyl;
R₄ is —CH₂R₄ₐ, —CH₂CH₂R₄ₐ, —CH₂CHR₄ₐR_{4d}, —CHR₄ₐR_{4b}, or —CR₄ₐR_{4b}R_{4c};
R₄ₐ and R_{4b} are independently:
(i) —CN or C₁₋₆ alkyl substituted with zero to 4 substituents independently selected from F, Cl, —CN, —OH, —OCH₃, —SCH₃, C₁₋₃ fluoroalkoxy, —NRₐRₐ, —S(O)₂Rₑ, or —NRₐS(O)₂Rₑ;
(ii) C₃₋₈ carbocyclyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₄ hydroxyalkyl, —(CH₂)₁₋₂O(C₁₋₃ alkyl), C₁₋₄ alkoxy, C₁₋₃ fluoroalkoxy, C₁₋₃ cyanoalkoxy, —O(C₁₋₄ hydroxyalkyl), —O(CRₓRₓ)₁₋₃O(C₁₋₃ alkyl), C₁₋₃ fluoroalkoxy, —O(CH₂)₁₋₃NR_{c}R_{c}, —OCH₂CH—CH₂, —OCH₂C≡CH, —C(O)(C₁₋₄ alkyl), —C(O)OH, —C(O)O(C₁₋₄ alkyl), —NR_{c}R_{c}, —CH₂NRₐRₐ, —NRₐS(O)₂(C₁₋₃ alkyl), —NRₐC(O)(C₁₋₃ alkyl), —(CRₓRₓ)₀₋₂NRₐC(O)O(C₁₋₄ alkyl), —P(O)(C₁₋₃ alkyl)₂, —S(O)₂(C₁₋₃ alkyl), —(CRₓRₓ)₁₋₂(C₃₋₄ cycloalkyl), —(CRₓRₓ)₁₋₂(morpholinyl), (CRₓRₓ)₁₋₂(difluoromorpholinyl), —(CRₓRₓ)₁₋₂(dimethylmorpholinyl), —(CRₓRₓ)₁₋₂(oxaazabicyclo[2.2.1]heptanyl), (CRₓRₓ)₁₋₂(oxaazaspiro[3.3]heptanyl), —(CRₓRₓ)₁₋₂(methylpiperazinonyl), —(CRₓRₓ)₁₋₂(acetylpiperazinyl), —(CRₓRₓ)₁₋₂(piperidinyl), —(CRₓRₓ)₁₋₂(difluoropiperidinyl), —(CRₓRₓ)₁₋₂(methoxypiperidinyl), —(CRₓRₓ)₁₋₂(hydroxypiperidinyl), —O(CRₓRₓ)₀₋₂(C₃₋₆ cycloalkyl), —O(CRₓRₓ)₀₋₂(methylcyclopropyl), —O(CRₓRₓ)₀₋₂((ethoxycarbonyl)cyclopropyl), —O(CRₓRₓ)₀₋₂(oxetanyl), —O(CRₓRₓ)₀₋₂(methylazetidinyl), —O(CRₓRₓ)₀₋₂(tetrahydropyranyl), —O(CRₓRₓ)₁₋₂(morpholinyl), —O(CRₓRₓ)₀₋₂(thiazolyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, dioxolanyl, pyrrolidinonyl, and R_{d}; or
(iii) C₁₋₄ alkyl substituted with one cyclic group selected from C₃₋₆ cycloalkyl, 4- to 10-membered heterocyclyl, mono- or bicyclic aryl, or 5- to 10-membered heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH—CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl;
or R₄ₐ and R_{4b} together with the carbon atom to which they are attached form a C₃₋₆ cycloalkyl or a 3- to 6-membered heterocyclyl, each substituted with zero to 3 R_{f};
each R_{f} is independently F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, and —NR_{c}R_{c};
R_{4c} is C₁₋₆ alkyl or C₃₋₆ cycloalkyl, each substituted with zero to 4 substituents independently selected from F, Cl, —OH, C₁₋₂ alkoxy, C₁₋₂ fluoroalkoxy, and —CN;
R_{4d} is —OCH₃;
each R_{c} is independently H or C₁₋₂ alkyl;
R_{d} is phenyl substituted with zero to 1 substituent selected from F, Cl, —CN, —CH₃, and —OCH₃;
each R₅ is independently —CN, C₁₋₆ alkyl substituted with zero to 4 R_{g}, C₂₋₄ alkenyl substituted with zero to 4 R_{g}, C₂₋₄ alkynyl substituted with zero to 4 R_{g}, C₃₋₄ cycloalkyl substituted with zero to 4 R_{g}, phenyl substituted with zero to 4 R_{g}, oxadiazolyl substituted with zero to 3 R_{g}, pyridinyl substituted with zero to 4 R_{g}, —(CH₂)₁₋₂(4- to 10-membered heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each R_{g} is independently F, Cl, —CN, —OH, C₁₋₃ alkoxy, C₁₋₃ fluoroalkoxy, —O(CH₂)₁₋₂O(C₁₋₂ alkyl), or —NR_{c}R_{c};
m is zero, 1, 2, or 3; and
n is zero, 1, or 2.

In some embodiments, the provided combination therapy of a T cell therapy and a DGK inhibitor involves administration or use of an inhibitor of DGKα and/or DGKζ that is a compound of Formula (II):

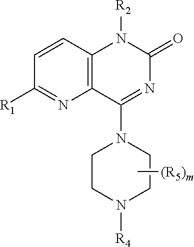

or a salt thereof, wherein:
R₁ is H, F, Cl, Br, —CN, C₁₋₃ alkyl substituted with zero to 4 R₁ₐ, C₃₋₄ cycloalkyl substituted with zero to 4 R₁ₐ, C₁₋₃ alkoxy substituted with zero to 4 R₁ₐ, —NRₐRₐ, —S(O)ₙRₑ, or —P(O)RₑRₑ;
each R₁ₐ is independently F, Cl, —CN, —OH, —OCH₃, or —NRₐRₐ;
each Rₐ is independently H or C₁₋₃ alkyl;
each Rₑ is independently C₃₋₄ cycloalkyl or C₁₋₃ alkyl substituted with zero to 4 R₁ₐ;
R₂ is H, C₁₋₃ alkyl substituted with zero to 4 R₂ₐ, or C₃₋₄ cycloalkyl substituted with zero to 4 R₂ₐ;
each R₂ₐ is independently F, Cl, —CN, —OH, —O(C₁₋₂ alkyl), C₃₋₄ cycloalkyl, C₃₋₄ alkenyl, or C₃₋₄ alkynyl;
R₄ is —CH₂R₄ₐ, —CH₂CH₂R₄ₐ, —CH₂CHR₄ₐR_{4d}, —CHR₄ₐR_{4b}, or —CR₄ₐR_{4b}R_{4c};
R₄ₐ and R_{4b} are independently:
(i) C₁₋₆ alkyl substituted with zero to 4 substituents independently selected from F, Cl, —CN, —OH, —OCH₃, —SCH₃, C₁₋₃ fluoroalkoxy, —NRₐRₐ, —S(O)₂Rₑ, or —NRₐS(O)₂Rₑ;
(ii) C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, C₁₋₄ hydroxyalkyl, —(CH₂)₁₋₂O(C₁₋₃ alkyl), C₁₋₄ alkoxy, —O(C₁₋₄ hydroxyalkyl), —O(CH₂)₁₋₃O(C₁₋₃ alkyl), C₁₋₃ fluoroalkoxy, —O(CH₂)₁₋₃NR_{c}R_{c}, —OCH₂CH—CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₃ alkyl)₂, —S(O)₂(C₁₋₃ alkyl), —O(CH₂)₁₋₂(C₃₋₆ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or
(iii) C₁₋₄ alkyl substituted with one cyclic group selected from C₃₋₆ cycloalkyl, heterocyclyl, aryl, and heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl;
or R₄ₐ and R_{4b} together with the carbon atom to which they are attached form a C₃₋₆ cycloalkyl or a 3- to 6-membered heterocyclyl, each substituted with zero to 3 R_{f};
each R_{f} is independently F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, and —NR_{c}R_{c};
R_{4c} is C₁₋₆ alkyl or C₃₋₆ cycloalkyl, each substituted with zero to 4 substituents independently selected from F, Cl, —OH, C₁₋₂ alkoxy, C₁₋₂ fluoroalkoxy, and —CN;
R_{4d} is —OCH₃;
each R_{c} is independently H or C₁₋₂ alkyl;
R_{d} is phenyl substituted with zero to 1 substituent selected from F, Cl, —CN, —CH₃, and —OCH₃;
each R₅ is independently —CN, C₁₋₆ alkyl substituted with zero to 4 R_{g}, C₂₋₄ alkenyl substituted with zero to 4 R_{g}, C₂₋₄ alkynyl substituted with zero to 4 R_{g}, C₃₋₄ cycloalkyl substituted with zero to 4 R_{g}, phenyl substituted with zero to 4 R_{g}, oxadiazolyl substituted with zero to 3 R_{g}, pyridinyl substituted with zero to 4 R_{g}, —(CH₂)₁₋₂(4- to 10-membered heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each R_{g} is independently F, Cl, —CN, —OH, C₁₋₃ alkoxy, C₁₋₃ fluoroalkoxy, —O(CH₂)₁₋₂O(C₁₋₂ alkyl), or —NR_{c}R_{c};
m is zero, 1, 2, or 3; and
n is zero, 1, or 2.

In one embodiment, a compound of Formula (II) or a salt thereof is administered wherein: R₁ is H, F, Cl, Br, —CN, —OH, C₁₋₃ alkyl substituted with zero to 4 R₁ₐ, cyclopropyl substituted with zero to 3 R₁ₐ, C₁₋₃ alkoxy substituted with zero to 3 R₁ₐ, —NRₐRₐ, —S(O)ₙCH₃, or —P(O)(CH₃)₂; R₂ is H or C₁₋₂ alkyl substituted with zero to 2 R₂ₐ; each R₂ₐ is independently F, Cl, —CN, —OH, —O(C₁₋₂ alkyl), cyclopropyl, C₃₋₄ alkenyl, or C₃₋₄ alkynyl; R₄ₐ and R_{4b} are independently: (i) —CN or C₁₋₄ alkyl substituted with zero to 4 substituents independently selected from F, Cl, —CN, —OH, —OCH₃, —SCH₃, C₁₋₃ fluoroalkoxy, and —NRₐRₐ; (ii) C₃₋₆ carbocyclyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₂ hydroxyalkyl, —CH₂NRₐRₐ, —(CH₂)₁₋₂O(C₁₋₂ alkyl), —(CH₂)₁₋₂NRₓC(O)O(C₁₋₂ alkyl), C₁₋₄ alkoxy, —O(C₁₋₄ hydroxyalkyl), —O(CRₓRₓ)₁₋₂O(C₁₋₂ alkyl), C₁₋₃ fluoroalkoxy, C₁₋₃ cyanoalkoxy, —O(CH₂)₁₋₂NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —(CH₂)₁₋₂(C₃₋₄ cycloalkyl), —CRₓRₓ(morpholinyl), —CRₓRₓ(difluoromorpholinyl), —CRₓRₓ(dimethylmorpholinyl), —CRₓRₓ(oxaazabicyclo[2.2.1]heptanyl), —CRₓRₓ(oxaazaspiro[3.3]heptanyl), —CRₓRₓ(methylpiperazinonyl), —CRₓRₓ(acetylpiperazinyl), —CRₓRₓ(piperidinyl), —CRₓRₓ(difluoropiperidinyl), —CRₓRₓ(methoxypiperidinyl), —CRₓRₓ(hydroxypiperidinyl), —O(CH₂)₀₋₂(C₃₋₄ cycloalkyl), —O(CH₂)₀₋₂(methylcyclopropyl), —O(CH₂)₀₋₂((ethoxycarbonyl)cyclopropyl), —O(CH₂)₀₋₂(oxetanyl), —O(CH₂)₀₋₂(methylazetidinyl), —O(CH₂)₁₋₂(morpholinyl), —O(CH₂)₀₋₂(tetrahydropyranyl), —O(CH₂)₀₋₂(thiazolyl), cyclopropyl, cyanocyclopropyl, methylazetidinyl, acetylazetidinyl, (tert-butoxycarbonyl)azetidinyl, dioxolanyl, pyrrolidinonyl, triazolyl, tetrahydropyranyl, morpholinyl, thiophenyl, methylpiperidinyl, and R_{d}; or (iii) C₁₋₃ alkyl substituted with one cyclic group selected from C₃₋₆ cycloalkyl, 4- to 10-membered heterocyclyl, phenyl, and heteroaryl, said cyclic group substituted with zero to 3 substituents independently selected from F, Cl, Br, —OH, —CN, C₁₋₃ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₄ cycloalkyl; or R₄ₐ and R_{4b} together with the carbon atom to which they are attached, form a C₃₋₆ cycloalkyl or a 3- to 6-membered heterocyclyl, each substituted with zero to 3 R_{f}; each R_{f} is independently F, Cl, Br, —OH, —CN, C₁₋₄ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₄ alkyl, C₁₋₂ fluoroalkyl, C₁₋₃ alkoxy, C₁₋₂ fluoroalkoxy, and —NR_{c}R_{c}; R_{4c} is C₁₋₄ alkyl or C₃₋₆ cycloalkyl, each substituted with zero to 4 substituents independently selected from F, Cl, —OH, C₁₋₂ alkoxy, C₁₋₂ fluoroalkoxy, and —CN; each R₅ is independently —CN, C₁₋₅ alkyl substituted with zero to 4 R_{g}, C₂₋₃ alkenyl substituted with zero to 4 R_{g}, C₂₋₃ alkynyl substituted with zero to 4 R_{g}, C₃₋₄ cycloalkyl substituted with zero to 4 R_{g}, phenyl substituted with zero to 3 R_{g}, oxadiazolyl substituted with zero to 3 R_{g}, pyridinyl substituted with zero to 3 R_{g}, —(CH₂)₁₋₂(4- to 10-membered heterocyclyl substituted with zero to 4 R_{g}), —(CH₂)₁₋₂NR_{c}C(O)(C₁₋₄ alkyl), (CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each Rₓ is independently H or —CH₃; and m is 1, 2, or 3.

In one embodiment, a compound of Formula (II) or a salt thereof is administered wherein: R₁ is H, F, Cl, Br, —CN, C₁₋₃ alkyl substituted with zero to 4 R₁ₐ, cyclopropyl substituted with zero to 3 R₁ₐ, C₁₋₃ alkoxy substituted with zero to 3 R₁ₐ, —NRₐRₐ, —S(O)ₙCH₃, or —P(O)(CH₃)₂; each R₁ₐ is independently F, Cl, or —CN; each Rₐ is independently H or C₁₋₃ alkyl; R₂ is H or C₁₋₂ alkyl substituted with zero to 2 R₂ₐ; each R₂ₐ is independently F, Cl, —CN, —OH, —O(C₁₋₂ alkyl), cyclopropyl, C₃₋₄ alkenyl, or C₃₋₄ alkynyl; R₄ₐ and R_{4b} are independently: (i) C₁₋₄ alkyl substituted with zero to 4 substituents independently selected from F, Cl, —CN, —OH, —OCH₃, —SCH₃, C₁₋₃ fluoroalkoxy, and —NRₐRₐ; (ii) C₃₋₆ cycloalkyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, —CH₂OH, —(CH₂)₁₋₂O(C₁₋₂ alkyl), C₁₋₄ alkoxy, —O(C₁₋₄ hydroxyalkyl), —O(CH₂)₁₋₂O(C₁₋₂ alkyl), C₁₋₃ fluoroalkoxy, —O(CH₂)₁₋₂NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —O(CH₂)₁₋₂(C₃₋₄ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or (iii) C₁₋₃ alkyl substituted with one cyclic group selected from C₃₋₆ cycloalkyl, heterocyclyl, phenyl, and heteroaryl, said cyclic group substituted with zero to 3 substituents independently selected from F, Cl, Br, —OH, —CN, C₁₋₃ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₄ cycloalkyl; or R₄ₐ and R_{4b} together with the carbon atom to which they are attached, form a C₃₋₆ cycloalkyl or a 3- to 6-membered heterocyclyl, each substituted with zero to 3 R_{f}; each R_{f} is independently F, Cl, Br, —OH, —CN, ═O, C₁₋₄ alkyl, C₁₋₂ fluoroalkyl, C₁₋₃ alkoxy, C₁₋₂ fluoroalkoxy, —OCH₂CH—CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₄ alkyl, C₁₋₂ fluoroalkyl, C₁₋₃ alkoxy, C₁₋₂ fluoroalkoxy, and —NR_{c}R_{c}; R_{4c} is C₁₋₄ alkyl or C₃₋₆ cycloalkyl, each substituted with zero to 4 substituents independently selected from F, Cl, —OH, C₁₋₂ alkoxy, C₁₋₂ fluoroalkoxy, and —CN; each R₅ is independently —CN, C₁₋₅ alkyl substituted with zero to 4 R_{g}, C₂₋₃ alkenyl substituted with zero to 4 R_{g}, C₂₋₃ alkynyl substituted with zero to 4 R_{g}, C₃₋₄ cycloalkyl substituted with zero to 4 R_{g}, phenyl substituted with zero to 3 R_{g}, oxadiazolyl substituted with zero to 3 R_{g}, pyridinyl substituted with zero to 3 R_{g}, —(CH₂)₁₋₂(heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —C(O)(C₁₋₄ alkyl), —(CH₂)₁₋₂NR_{c}S(O)₂(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl); and m is 1, 2, or 3.

In one embodiment, a compound of Formula (II) or a salt thereof is administered wherein: R₁ is Cl or —CN; R₂ is —CH₃; R₄ is —CH₂R₄ₐ or —CHR₄ₐR_{4b}; R₄ₐ is cyclopropyl, cyclobutyl, cyclohexyl, bicyclo[1.1.1]pentanyl, phenyl, pyridinyl, pyrimidinyl, oxadiazolyl, benzo[d][1,3]dioxolyl, or oxodihydrobenzo[d]oxazolyl, each substituted with zero to 3 substituents independently selected from F, Cl, —CN, —CH₃, —CH(CH₃)₂, —CF₃, —OCH₃, —OCH(CH₃)₂, —OCHF₂, —OCF₃, —OCH₂(cyclopropyl), and cyclopropyl; R_{4b} is: (i) —CH₃ and —CH₂CH₃; or (ii)phenyl, isoxazolyl, oxadiazolyl, or thiazolyl, each substituted with zero to 3 substituents independently selected from F, Cl, —CH₃, —C(CH₃)₃, —CF₃, —OCF₃, and cyclopropyl; each R₅ is independently —CH₃, —CH₂CH₃, —CH₂OH, or —CH₂OCH₃; and m is 2.

In one embodiment, a compound of Formula (II) or a salt thereof is administered wherein: R₁ is Cl, —CN, —OH, —CHF₂, —CH₂OH, —CH₂OCH₃, —OCH₃, —OCH₂CH₃, —OCHF₂, —OCH₂CH₂OCH₃, or —OCH₂CH₂N(CH₃)₂; R₂ is H, —CH₃, or —CD₃; R₄ is —CH₂R₄ₐ or —CHR₄ₐR_{4b}; R₄ₐ is cyclohexyl, phenyl, pyridinyl, pyrimidinyl, oxadiazolyl, benzo[d][1,3]dioxolyl, or oxodihydrobenzo[d]oxazolyl, each substituted with zero to 3 substituents independently selected from F, Cl, Br, —CN, —CH₃, —CH(CH₃)₂, —C(CH₃)₃, —CH₂OH, —CHF₂, —CF₃, —CH₂Br, —CH₂NH₂, —CH₂NHC(O)OCH₃, —C(CH₃)₂CN, —OCH₃, —OCD₃, —OCH₂CH₃, —OCH(CH₃)₂, —OCHF₂, —OCF₃, —OCH₂CH₂CF₃, —OC(CH₃)₂CN, —OC(CH₃)₂CH₂OH, —OC(CH₃)₂CH₂OCH₃, —N(CH₃)₂, —C(O)OCH₃, cyclopropyl, cyanocyclopropyl, methylcyclopropyl, —O(cyclopropyl), —O((ethoxycarbonyl)cyclopropyl), morpholinyl, pyrrolidinonyl, tetrahydropyranyl, dioxolanyl, —CH₂(morpholinyl), —CH₂(difluoromorpholinyl), —CH₂(dimethylmorpholinyl), —CH₂(oxaazabicyclo[2.2.1]heptanyl), —CH₂(oxaazaspiro[3.3]heptanyl), —CH₂(methylpiperazinonyl), —CH₂(acetylpiperazinyl), —CH₂(piperidinyl), —CH₂(difluoropiperidinyl), —CH₂(methoxypiperidinyl), —CH₂(hydroxypiperidinyl), —C(CH₃) 2 (morpholinyl), —OCH₂(cyclopropyl), —OCH₂(methylcyclopropyl), —OCH₂(methylazetidinyl), —OCH₂(oxetanyl), —OCH₂(tetrahydropyranyl), —OCH₂(thiazolyl), or —OCH₂CH₂(cyclopropyl); R_{4b} is: (i) —CN, —CH₃, —CH₂CH₃, —CH₂CH₂CH₃, or —CH(CH₃)₂; or (ii)phenyl, isoxazolyl, oxadiazolyl, thiazolyl, or triazolyl, each substituted with zero to 3 substituents independently selected from F, Cl, Br, —CH₃, —C(CH₃)₃, —CF₃, —OCF₃, and cyclopropyl; each R₅ is independently —CH₃, —CH₂CH₃, —CH₂CH₂CH₃, —CH₂OH, —CH₂OCH₃, —CH₂OCH₂CH₃, —CH₂NH₂, —CH₂N₃, or —CH₂NHC(O)OCH₃; and m is 2.

In one embodiment, a compound of Formula (II) or a salt thereof is administered wherein R₁ is H, F, Cl, Br, —CN, —OH, C₁₋₃ alkyl substituted with zero to 4 R₁ₐ, cyclopropyl substituted with zero to 3 R₁ₐ, C₁₋₃ alkoxy substituted with zero to 3 R₁ₐ, —NRₐRₐ, —S(O)ₙCH₃, or —P(O)(CH₃)₂. In some embodiments, a compound of Formula (II) or a salt thereof is administered wherein R₁ is Cl, —CN, —OH, —CHF₂, —CH₂OH, —CH₂OCH₃, —OCH₃, —OCH₂CH₃, —OCHF₂, —OCH₂CH₂OCH₃, or —OCH₂CH₂N(CH₃) 2.

In one embodiment, a compound of Formula (II) or a salt thereof is administered wherein R₁ is H, F, Cl, Br, —CN, C₁₋₃ alkyl substituted with zero to 4 R₁ₐ, cyclopropyl substituted with zero to 3 R₁ₐ, C₁₋₃ alkoxy substituted with zero to 3 R₁ₐ, —NRₐRₐ, —S(O)ₙCH₃, or —P(O)(CH₃)₂. In some embodiments, a compound of Formula (II) or a salt thereof is administered wherein R₁ is H, F, Cl, Br, —CN, —CH₃, cyclopropyl, —OCH₃, or —NH₂. In some embodiments, a compound of Formula (II) or a salt thereof is administered wherein R₁ is Cl or —CN. In some embodiments, a compound of Formula (II) or a salt thereof is administered wherein R₁ is —CN.

In one embodiment, a compound of Formula (II) or a salt thereof is administered wherein R₂ is H, C₁₋₂ alkyl substituted with zero to 4 R₂ₐ, or C₃₋₄ cycloalkyl substituted with zero to 2 R₂ₐ. In some embodiments, a compound of Formula (II) or a salt thereof is administered wherein R₂ is H or C₁₋₂ alkyl substituted with zero to 2 R₂ₐ. In some embodiments, a compound of Formula (II) or a salt thereof is administered wherein R₂ is H or —CH₃. In some embodiments, a compound of Formula (II) or a salt thereof is administered wherein R₁ is —CH₃. In some embodiments, a compound of Formula (II) or a salt thereof is administered wherein R₂ is H, —CH₃, or —CD₃.

In one embodiment, a compound of Formula (II) or a salt thereof is administered wherein R₂ is H.

In one embodiment, a compound of Formula (II) or a salt thereof is administered wherein R₂ is —CH₃.

In one embodiment, a compound of Formula (II) or a salt thereof is administered wherein R₂ is —CD₃.

In one embodiment, a compound of Formula (II) or a salt thereof is administered wherein R₄ is —CH₂R₄ₐ or —CH₂CH₂R₄ₐ. In some embodiments, a compound of Formula (II) or a salt thereof is administered wherein R₄ is —CH₂R₄ₐ or —CD₂R₄ₐ. AIn some embodiments, a compound of Formula (II) or a salt thereof is administered wherein R₄ₐ is phenyl, pyridinyl, tetrahydropyranyl, benzoxazinyl, benzo[d][1,3]dioxolyl, benzoxazinonyl, indazolyl, indolyl, or quinolinyl, each substituted with zero to 3 substituents independently selected from F, Cl, Br, —CN, —OH, —CH₃, —CH₂CH₃, —CH(CH₃)₂, —C(CH₃)₃, —CHF₂, —CF₃, —OCH₃, —OCH₂CH₃, —OCH(CH₃)₂, —OCHF₂, —OCF₃, —C(O)CH₃, —C(O)OC(CH₃)₃, —N(CH₃)₂, cyanocyclopropyl, and phenyl. In some embodiments, a compound of Formula (II) or a salt thereof is administered wherein R₄ₐ is phenyl, pyridinyl, or benzo[d][1,3]dioxolyl, each substituted with zero to 3 substituents independently selected from F, Cl, —CN, —CH₃, —CH(CH₃)₂, —CF₃, —OCH₃, —OCH(CH₃)₂, —OCHF₂, —OCF₃, —OCH₂(cyclopropyl), and cyclopropyl.

In one embodiment, a compound of Formula (II) or a salt thereof is administered wherein R₄ is —CH₂R₄ₐ. In some embodiments, a compound of Formula (II) or a salt thereof is administered wherein R₄ₐ is phenyl, pyridinyl, tetrahydropyranyl, benzoxazinyl, benzo[d][1,3]dioxolyl, benzoxazinonyl, indazolyl, indolyl, or quinolinyl, each substituted with zero to 3 substituents independently selected from F, Cl, Br, —CN, —OH, —CH₃, —CH₂CH₃, —CH(CH₃)₂, —C(CH₃)₃, —CHF₂, —CF₃, —OCH₃, —OCH₂CH₃, —OCH(CH₃)₂, —OCHF₂, —OCF₃, —C(O)CH₃, —C(O)OC(CH₃)₃, —N(CH₃)₂, cyanocyclopropyl, and phenyl. In some embodiments, a compound of Formula (II) or a salt thereof is administered wherein R₄ₐ is phenyl, pyridinyl, or benzo[d][1,3]dioxolyl, each substituted with zero to 3 substituents independently selected from F, Cl, —CN, —CH₃, —CH(CH₃)₂, —CF₃, —OCH₃, —OCH(CH₃)₂, —OCHF₂, —OCF₃, —OCH₂(cyclopropyl), and cyclopropyl.

In one embodiment, a compound of Formula (II) or a salt thereof is administered wherein R₄ is —CH₂R₄ₐ; and R₄ₐ is C₃₋₈ carbocyclyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₄ hydroxyalkyl, —(CH₂)₁₋₂O(C₁₋₃ alkyl), C₁₋₄ alkoxy, C₁₋₃ fluoroalkoxy, C₁₋₃ cyanoalkoxy, —O(C₁₋₄ hydroxyalkyl), —O(CRₓRₓ)₁₋₃O(C₁₋₃ alkyl), C₁₋₃ fluoroalkoxy, —O(CH₂)₁₋₃NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —CH₂NRₐRₐ, —NRₐS(O)₂(C₁₋₃ alkyl), —NRₐC(O)(C₁₋₃ alkyl), (CRₓRₓ)₀₋₂NRₐC(O)O(C₁₋₄ alkyl), —P(O)(C₁₋₃ alkyl)₂, —S(O)₂(C₁₋₃ alkyl), —(CRₓRₓ)₁₋₂(C₃₋₄ cycloalkyl), —(CRₓRₓ)₁₋₂(morpholinyl), (CRₓRₓ)₁₋₂(difluoromorpholinyl), —(CRₓRₓ)₁₋₂(dimethylmorpholinyl), (CRₓRₓ)₁₋₂(oxaazabicyclo[2.2.1]heptanyl), (CRₓRₓ)₁₋₂(oxaazaspiro[3.3]heptanyl), —(CRₓRₓ)₁₋₂(methylpiperazinonyl), —(CRₓRₓ)₁₋₂(acetylpiperazinyl), —(CRₓRₓ)₁₋₂(piperidinyl), —(CRₓRₓ)₁₋₂(difluoropiperidinyl), —(CRₓRₓ)₁₋₂(methoxypiperidinyl), —(CRₓRₓ)₁₋₂(hydroxypiperidinyl), —O(CRₓRₓ)₀₋₂(C₃₋₆ cycloalkyl), —O(CRₓRₓ)₀₋₂(methylcyclopropyl), —O(CRₓRₓ)₀₋₂((ethoxycarbonyl)cyclopropyl), —O(CRₓRₓ)₀₋₂(oxetanyl), —O(CRₓRₓ)₀₋₂(methylazetidinyl), —O(CRₓRₓ)₀₋₂(tetrahydropyranyl), —O(CRₓRₓ)₁₋₂(morpholinyl), —O(CRₓRₓ)₀₋₂(thiazolyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, dioxolanyl, pyrrolidinonyl, and R_{d}.

In one embodiment, a compound of Formula (II) or a salt thereof is administered wherein R₄ is —CH₂R₄ₐ; and R₄ₐ is C₃₋₆ cycloalkyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₂ hydroxyalkyl, —CH₂NRₐRₐ, —(CH₂)₁₋₂O(C₁₋₂ alkyl), —(CH₂)₁₋₂NRₓC(O)O(C₁₋₂ alkyl), C₁₋₄ alkoxy, —O(C₁₋₄ hydroxyalkyl), —O(CRₓRₓ)₁₋₂O(C₁₋₂ alkyl), C₁₋₃ fluoroalkoxy, C₁₋₃ cyanoalkoxy, —O(CH₂)₁₋₂NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —(CH₂)₁₋₂(C₃₋₄ cycloalkyl), —CRₓRₓ(morpholinyl), —CRₓRₓ(difluoromorpholinyl), —CRₓRₓ(dimethylmorpholinyl), —CRₓRₓ(oxaazabicyclo[2.2.1]heptanyl), —CRₓRₓ(oxaazaspiro[3.3]heptanyl), —CRₓRₓ(methylpiperazinonyl), —CRₓRₓ(acetylpiperazinyl), —CRₓRₓ(piperidinyl), —CRₓRₓ(difluoropiperidinyl), —CRₓRₓ(methoxypiperidinyl), —CRₓRₓ(hydroxypiperidinyl), —O(CH₂)₀₋₂(C₃₋₄ cycloalkyl), —O(CH₂)₀₋₂(methylcyclopropyl), —O(CH₂)₀₋₂((ethoxycarbonyl)cyclopropyl), —O(CH₂)₀₋₂(oxetanyl), —O(CH₂)₀₋₂(methylazetidinyl), —O(CH₂)₁₋₂(morpholinyl), —O(CH₂)₀₋₂(tetrahydropyranyl), —O(CH₂)₀₋₂(thiazolyl), cyclopropyl, cyanocyclopropyl, methylazetidinyl, acetylazetidinyl, (tert-butoxycarbonyl)azetidinyl, dioxolanyl, pyrrolidinonyl, triazolyl, tetrahydropyranyl, morpholinyl, thiophenyl, methylpiperidinyl, and R_{d}. In some embodiments, a compound of Formula (II) or a salt thereof is administered wherein R₄ₐ is cyclohexyl, phenyl, or benzo[d][1,3]dioxolyl, each substituted with 1 to 3 substituents independently selected from F, Cl, —CH(CH₃)₂, —CF₃, —OCH₂CH₃, —OCF₃, cyclopropyl, and —OCH₂(cyclopropyl).

In one embodiment, a compound of Formula (II) or a salt thereof is administered wherein R₄ is —CHR₄ₐR_{4b}; R₄ₐ is: (i) C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, C₁₋₄ hydroxyalkyl, —(CH₂)₁₋₂O(C₁₋₃ alkyl), C₁₋₄ alkoxy, —O(C₁₋₄ hydroxyalkyl), —O(CH₂)₁₋₃O(C₁₋₃ alkyl), C₁₋₃ fluoroalkoxy, —O(CH₂)₁₋₃NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₃ alkyl)₂, —S(O)₂(C₁₋₃ alkyl), —O(CH₂)₁₋₂(C₃₋₆ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or (ii) C₁₋₄ alkyl substituted with one cyclic group selected from C₃₋₆ cycloalkyl, heterocyclyl, aryl, and heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl; and R_{4b} is phenyl or heteroaryl, each substituted with zero to 4 substituents independently selected from F, Cl, Br, —CN, —OH, C₁₋₆ alkyl, C₁₋₃ fluoroalkyl, C₁₋₄ hydroxyalkyl, —(CH₂)₁₋₂O(C₁₋₃ alkyl), C₁₋₄ alkoxy, —O(C₁₋₄ hydroxyalkyl), —O(CH₂)₁₋₃O(C₁₋₃ alkyl), C₁₋₃ fluoroalkoxy, —O(CH₂)₁₋₃NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₃ alkyl)₂, —S(O)₂(C₁₋₃ alkyl), —O(CH₂)₁₋₂(C₃₋₆ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, and methylpiperidinyl. In some embodiments, a compound of Formula (II) or a salt thereof is administered wherein R₄ₐ is (i) C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, —CH₂OH, —(CH₂)₁₋₂O(C₁₋₂ alkyl), C₁₋₄ alkoxy, —O(C₁₋₄ hydroxyalkyl), —O(CH₂)₁₋₂O(C₁₋₂ alkyl), C₁₋₃ fluoroalkoxy, —O(CH₂)₁₋₂NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —O(CH₂)₁₋₂(C₃₋₄ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or (ii) C₁₋₃ alkyl substituted with one cyclic group selected from C₃₋₆ cycloalkyl, heterocyclyl, phenyl, and heteroaryl, said cyclic group substituted with zero to 3 substituents independently selected from F, Cl, Br, —OH, —CN, C₁₋₃ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₄ cycloalkyl; and R_{4b} is phenyl, isoxazolyl, oxadiazolyl, or thiazolyl, each substituted with zero to 3 substituents independently selected from F, Cl, —CH₃, —C(CH₃)₃, —CF₃, —OCF₃, and cyclopropyl. In some embodiments, a compound of Formula (II) or a salt thereof is administered wherein R₄ₐ is phenyl, pyridinyl, or benzo[d][1,3]dioxolyl, each substituted with zero to 3 substituents independently selected from F, Cl, —CN, —CH₃, —CH(CH₃)₂, —CF₃, —OCH₃, —OCH(CH₃)₂, —OCHF₂, —OCF₃, —OCH₂(cyclopropyl), and cyclopropyl; and R_{4b} is phenyl, isoxazolyl, oxadiazolyl, or thiazolyl, each substituted with zero to 3 substituents independently selected from F, Cl, —CH₃, —C(CH₃)₃, —CF₃, —OCF₃, and cyclopropyl.

In one embodiment, a compound of Formula (II) or a salt thereof is administered wherein R₄ is —CHR₄ₐR_{4b}; R₄ₐ is: (i) C₃₋₆ carbocyclyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₄ hydroxyalkyl, —(CH₂)₁₋₂O(C₁₋₃ alkyl), C₁₋₄ alkoxy, C₁₋₃ fluoroalkoxy, C₁₋₃ cyanoalkoxy, —O(C₁₋₄ hydroxyalkyl), —O(CRₓRₓ)₁₋₃O(C₁₋₃ alkyl), C₁₋₃ fluoroalkoxy, —O(CH₂)₁₋₃NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —CH₂NRₐRₐ, —NRₐS(O)₂(C₁₋₃ alkyl), —NRₐC(O)(C₁₋₃ alkyl), —(CRₓRₓ)₀₋₂NR₂C(O)O(C₁₋₄ alkyl), —P(O)(C₁₋₃ alkyl)₂, —S(O)₂(C₁₋₃ alkyl), —(CRₓRₓ)₁₋₂(C₃₋₄ cycloalkyl), —(CRₓRₓ)₁₋₂(morpholinyl), (CRₓRₓ)₁₋₂(difluoromorpholinyl), —(CRₓRₓ)₁₋₂(dimethylmorpholinyl), —(CRₓRₓ)₁₋₂(oxaazabicyclo[2.2.1]heptanyl), (CRₓRₓ)₁₋₂(oxaazaspiro[3.3]heptanyl), —(CRₓRₓ)₁₋₂(methylpiperazinonyl), —(CRₓRₓ)₁₋₂(acetylpiperazinyl), —(CRₓRₓ)₁₋₂(piperidinyl), —(CRₓRₓ)₁₋₂(difluoropiperidinyl), —(CRₓRₓ)₁₋₂(methoxypiperidinyl), —(CRₓRₓ)₁₋₂(hydroxypiperidinyl), —O(CRₓRₓ)₀₋₂(C₃₋₆ cycloalkyl), —O(CRₓRₓ)₀₋₂(methylcyclopropyl), —O(CRₓRₓ)₀₋₂((ethoxycarbonyl)cyclopropyl), —O(CRₓRₓ)₀₋₂(oxetanyl), —O(CRₓRₓ)₀₋₂(methylazetidinyl), —O(CRₓRₓ)₀₋₂(tetrahydropyranyl), —O(CRₓRₓ)₁₋₂(morpholinyl), —O(CRₓRₓ)₀₋₂(thiazolyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, dioxolanyl, pyrrolidinonyl, and R_{d}; or (ii) C₁₋₄ alkyl substituted with one cyclic group selected from C₃₋₆ carbocyclyl, 4- to 10-membered heterocyclyl, 6- to 10-membered aryl, or 5- to 10-membered heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl; and R_{4b} is phenyl, isoxazolyl, oxadiazolyl, thiazolyl, or triazolyl, each substituted with zero to 3 substituents independently selected from F, Cl, Br, —CH₃, —C(CH₃)₃, —CF₃, —OCF₃, and cyclopropyl.

In one embodiment, a compound of Formula (II) or a salt thereof is administered wherein R₄ is —CHR₄ₐR_{4b}; R₄ₐ is (i) C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, C₁₋₄ hydroxyalkyl, —(CH₂)₁₋₂O(C₁₋₃ alkyl), C₁₋₄ alkoxy, —O(C₁₋₄ hydroxyalkyl), —O(CH₂)₁₋₃O(C₁₋₃ alkyl), C₁₋₃ fluoroalkoxy, —O(CH₂)₁₋₃NR_{c}R_{c}, —OCH₂CH—CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NR₂C(O)(C₁₋₃ alkyl), —NRₐC(O)O(C₁₋₄ alkyl), —P(O)(C₁₋₃ alkyl)₂, —S(O)₂(C₁₋₃ alkyl), —O(CH₂)₁₋₂(C₃₋₆ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or (ii) C₁₋₄ alkyl substituted with one cyclic group selected from C₃₋₆ cycloalkyl, heterocyclyl, mono- or bicyclic aryl, and heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl; and R_{4b} is C₁₋₆ alkyl substituted with zero to 4 substituents independently selected from F, Cl, —CN, —OH, —OCH₃, —SCH₃, C₁₋₃ fluoroalkoxy, —NRₐRₐ, —S(O)₂Rₑ, or —NRₐS(O)₂Rₑ. In some embodiments, a compound of Formula (II) or a salt thereof is administered wherein R₄ₐ is C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, —CH₂OH, —(CH₂)₁₋₂O(C₁₋₂ alkyl), C₁₋₄ alkoxy, —O(C₁₋₄ hydroxyalkyl), —O(CH₂)₁₋₂O(C₁₋₂ alkyl), C₁₋₃ fluoroalkoxy, —O(CH₂)₁₋₂NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —O(CH₂)₁₋₂(C₃₋₄ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or (ii) C₁₋₃ alkyl substituted with one cyclic group selected from C₃₋₆ cycloalkyl, heterocyclyl, phenyl, and heteroaryl, said cyclic group substituted with zero to 3 substituents independently selected from F, Cl, Br, —OH, —CN, C₁₋₃ alkyl, C₁₋₂ fluoroalkyl, C₁₋₃ alkoxy, C₁₋₂ fluoroalkoxy, —OCH₂CH—CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₄ cycloalkyl; and R_{4b} is C₁₋₄ alkyl substituted with zero to 4 substituents independently selected from F, Cl, —CN, —OH, —OCH₃, —SCH₃, C₁₋₃ fluoroalkoxy, and —NRₐRₐ. In some embodiments, a compound of Formula (II) or a salt thereof is administered wherein R₄ₐ is phenyl, pyridinyl, or benzo[d][1,3]dioxolyl, each substituted with zero to 3 substituents independently selected from F, Cl, —CN, —CH₃, —CH(CH₃)₂, —CF₃, —OCH₃, —OCH(CH₃)₂, —OCHF₂, —OCF₃, —OCH₂(cyclopropyl), and cyclopropyl; and R_{4b} is —CH₃ and —CH₂CH₃.

In one embodiment, a compound of Formula (II) or a salt thereof is administered wherein R₄ is —CHR₄ₐR_{4b}; R₄ₐ is (i) C₃₋₈ carbocyclyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₄ hydroxyalkyl, —(CH₂)₁₋₂O(C₁₋₃ alkyl), C₁₋₄ alkoxy, C₁₋₃ fluoroalkoxy, C₁₋₃ cyanoalkoxy, —O(C₁₋₄ hydroxyalkyl), —O(CRₓRₓ)₁₋₃O(C₁₋₃ alkyl), C₁₋₃ fluoroalkoxy, —O(CH₂)₁₋₃NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —CH₂NRₐRₐ, —NRₐS(O)₂(C₁₋₃ alkyl), —NRₐC(O)(C₁₋₃ alkyl), —(CRₓRₓ)₀₋₂NRₐC(O)O(C₁₋₄ alkyl), —P(O)(C₁₋₃ alkyl)₂, —S(O)₂(C₁₋₃ alkyl), —(CRₓRₓ)₁₋₂(C₃₋₄ cycloalkyl), (CRₓRₓ)₁₋₂(morpholinyl), —(CRₓRₓ)₁₋₂(difluoromorpholinyl), —(CRₓRₓ)₁₋₂(dimethylmorpholinyl), —(CRₓRₓ)₁₋₂(oxaazabicyclo[2.2.1]heptanyl), (CRₓRₓ)₁₋₂(oxaazaspiro[3.3]heptanyl), —(CRₓRₓ)₁₋₂(methylpiperazinonyl), —(CRₓRₓ)₁₋₂(acetylpiperazinyl), —(CRₓRₓ)₁₋₂(piperidinyl), —(CRₓRₓ)₁₋₂(difluoropiperidinyl), —(CRₓRₓ)₁₋₂(methoxypiperidinyl), —(CRₓRₓ)₁₋₂(hydroxypiperidinyl), —O(CRₓRₓ)₀₋₂(C₃₋₆ cycloalkyl), —O(CRₓRₓ)₀₋₂(methylcyclopropyl), —O(CRₓRₓ)₀₋₂((ethoxycarbonyl)cyclopropyl), —O(CRₓRₓ)₀₋₂(oxetanyl), —O(CRₓRₓ)₀₋₂(methylazetidinyl), —O(CRₓRₓ)₀₋₂(tetrahydropyranyl), —O(CRₓRₓ)₁₋₂(morpholinyl), —O(CRₓRₓ)₀₋₂(thiazolyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, dioxolanyl, pyrrolidinonyl, and R_{d}; or (ii) C₁₋₄ alkyl substituted with one cyclic group selected from C₃₋₆ cycloalkyl, 4- to 10-membered heterocyclyl, mono- or bicyclic aryl, or 5- to 10-membered heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl; and R_{4b} is —CN or C₁₋₆ alkyl substituted with zero to 4 substituents independently selected from F, Cl, —CN, —OH, —OCH₃, —SCH₃, C₁₋₃ fluoroalkoxy, —NRₐRₐ, —S(O)₂Rₑ, or —NRₐS(O)₂Rₑ. In some embodiments, a compound of Formula (II) or a salt thereof is administered wherein R₄ₐ is (i) C₃₋₆ carbocyclyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₂ hydroxyalkyl, —CH₂NRₐRₐ, (CH₂)₁₋₂O(C₁₋₂ alkyl), —(CH₂)₁₋₂NRₓC(O)O(C₁₋₂ alkyl), C₁₋₄ alkoxy, —O(C₁₋₄ hydroxyalkyl), —O(CRₓRₓ)₁₋₂O(C₁₋₂ alkyl), C₁₋₃ fluoroalkoxy, C₁₋₃ cyanoalkoxy, —O(CH₂)₁₋₂NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —(CH₂)₁₋₂(C₃₋₄ cycloalkyl), —CRₓRₓ(morpholinyl), —CRₓRₓ(difluoromorpholinyl), —CRₓRₓ(dimethylmorpholinyl), —CRₓRₓ(oxaazabicyclo[2.2.1]heptanyl), —CRₓRₓ(oxaazaspiro[3.3]heptanyl), —CRₓRₓ(methylpiperazinonyl), —CRₓRₓ(acetylpiperazinyl), —CRₓRₓ(piperidinyl), —CRₓRₓ(difluoropiperidinyl), —CRₓRₓ(methoxypiperidinyl), —CRₓRₓ(hydroxypiperidinyl), —O(CH₂)₀₋₂(C₃₋₄ cycloalkyl), —O(CH₂)₀₋₂(methylcyclopropyl), —O(CH₂)₀₋₂((ethoxycarbonyl)cyclopropyl), —O(CH₂)₀₋₂(oxetanyl), —O(CH₂)₀₋₂(methylazetidinyl), —O(CH₂)₁₋₂(morpholinyl), —O(CH₂)₀₋₂(tetrahydropyranyl), —O(CH₂)₀₋₂(thiazolyl), cyclopropyl, cyanocyclopropyl, methylazetidinyl, acetylazetidinyl, (tert-butoxycarbonyl)azetidinyl, dioxolanyl, pyrrolidinonyl, triazolyl, tetrahydropyranyl, morpholinyl, thiophenyl, methylpiperidinyl, and R_{d}; or (ii) C₁₋₃ alkyl substituted with one cyclic group selected from C₃₋₆ cycloalkyl, 4- to 10-membered heterocyclyl, mono- or bicyclic aryl, or 5- to 10-membered heteroaryl, said cyclic group substituted with zero to 3 substituents independently selected from F, Cl, Br, —OH, —CN, C₁₋₃ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₄ cycloalkyl; and R_{4b} is —CN or C₁₋₄ alkyl substituted with zero to 4 substituents independently selected from F, Cl, —CN, —OH, —OCH₃, —SCH₃, C₁₋₃ fluoroalkoxy, and —NRₐRₐ.

In one embodiment, a compound of Formula (II) or a salt thereof is administered wherein R₄ is —CHR₄ₐR_{4b}; and R_{4b} is —CN, —CH₃, —CH₂CH₃, —CH₂CH₂CH₃, or —CH(CH₃)₂.

In one embodiment, a compound of Formula (II) or a salt thereof is administered wherein R₄ is —CHR₄ₐR_{4b}; and R_{4b} is —CN, —CH₃, or —CH₂CH₃.

In one embodiment, a compound of Formula (II) or a salt thereof is administered wherein R₄ is —CHR₄ₐR_{4b}; and R_{4b} is —CN.

In one embodiment, a compound of Formula (II) or a salt thereof is administered wherein R₄ is —CHR₄ₐR_{4b}; and R_{4b} is —CH₃ or —CH₂CH₃.

In one embodiment, a compound of Formula (II) or a salt thereof is administered wherein R₄ is —CHR₄ₐR_{4b}; and R_{4b} is —CH₃.

In one embodiment, a compound of Formula (II) or a salt thereof is administered wherein R₄ is —CHR₄ₐR_{4b}; and R_{4b} is —CH₂CH₃.

In one embodiment, a compound of Formula (II) or a salt thereof is administered wherein m is 1, 2, or 3; and each R₅ is independently —CN, C₁₋₅ alkyl substituted with zero to 4 R_{g}, C₂₋₃ alkenyl substituted with zero to 4 R_{g}, C₂₋₃ alkynyl substituted with zero to 4 R_{g}, C₃₋₄ cycloalkyl substituted with zero to 4 R_{g}, phenyl substituted with zero to 3 R_{g}, oxadiazolyl substituted with zero to 3 R_{g}, pyridinyl substituted with zero to 3 R_{g}, —(CH₂)₁₋₂(4- to 10-membered heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl). In some embodiments, a compound of Formula (II) or a salt thereof is administered wherein each R₅ is independently —CH₃, —CH₂CH₃, —CH₂OH, or —CH₂OCH₃.

In one embodiment, a compound of Formula (II) or a salt thereof is administered wherein m is zero.

In one embodiment, a compound of Formula (II) or a salt thereof is administered wherein m is 1, 2, or 3. In some embodiments, a compound of Formula (II) or a salt thereof is administered wherein m is 1 or 2. In some embodiments, a compound of Formula (II) or a salt thereof is administered wherein m is 1.

In one embodiment, a compound of Formula (II) or a salt thereof is administered wherein m is 2 or 3. In some embodiments, a compound of Formula (II) or a salt thereof is administered wherein m is 2.

In one embodiment, a compound of Formula (II) or a salt thereof is administered wherein m is 3.

In one embodiment, a compound of Formula (II) or a salt thereof is administered having the structure of Formula (III):

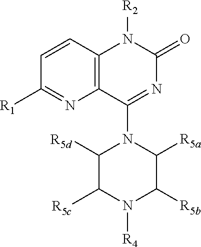

wherein one, two, or three of R₅ₐ, R_{5b}, R_{5c}, and R_{5d} are each R₅ and the remainder of R₅ₐ, R_{5b}, R_{5c}, and R_{5d} are each hydrogen. In some embodiments, a compound of Formula (III) or a salt thereof is administered wherein each R₅ is independently —CN, —CH₃, —CH₂CH₃, —CH(CH₃)₂, —CHC(CH₃)₂, —CH₂F, —C(CH₃)₂F, —CF(CH₃)CH(CH₃)₂, —CH₂OH, —C(CH₃)₂OH, —C(CH₃)(OH)CH(CH₃)₂, —CH₂OCH₃, —C(O)C(CH₃)₂, —C(O)OH, —C(O)OCH₃, —C(O)OC(CH₃)₂, —C(O)NH₂, —C(O)NH(cyclopropyl), —C(O)O(cyclopropyl), cyclopropyl, phenyl, methyloxadiazolyl, or methylpyridinyl. In some embodiments, a compound of Formula (III) or a salt thereof is administered wherein each R₅ is independently —CH₃, —CH₂CH₃, —CH₂CH₂CH₃, —CH₂OH, —CH₂OCH₃, —CH₂OCH₂CH₃, —CH₂NH₂, —CH₂N₃, or —CH₂NHC(O)OCH₃.

In one embodiment, a compound of Formula (II) or a salt thereof is administered having the structure of Formula (IV):

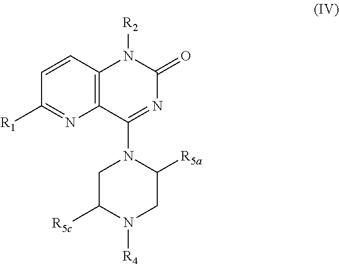

wherein R₅ₐ and R_{5c} are each R₅ and R_{5b} and R_{5d} are each hydrogen. In some embodiments, a compound of Formula (IV) or a salt thereof is administered wherein (i) R₅ₐ is —CH₃ or —CH₂CH₃ and R_{5c} is —CH₃ or —CH₂CH₃; or (ii) R₅ₐ is —CH₃ and R_{5c} is —CH₂OH or —CH₂OCH₃.

In one embodiment, a compound of Formula (IV) or a salt thereof is administered wherein R₅ₐ is —CH₃ and R_{5c} is —CH₃.

In one embodiment, a compound of Formula (IV) or a salt thereof is administered wherein R₅ₐ is —CH₃ and R_{5c} is —CH₂CH₃.

In one embodiment, a compound of Formula (IV) or a salt thereof is administered wherein R₅ₐ is —CH₂CH₃ and R_{5c} is —CH₃.

In one embodiment, a compound of Formula (IV) or a salt thereof is administered wherein R₅ₐ is —CH₂CH₃ and R_{5c} is —CH₂CH₃.

In one embodiment, a compound of Formula (IV) or a salt thereof is administered wherein R₅ₐ is —CH₃ and R_{5c} is —CH₂OH.

In one embodiment, a compound of Formula (IV) or a salt thereof is administered wherein R₅ₐ is —CH₃ and R_{5c} is —CH₂OCH₃.

In one embodiment, a compound of Formula (IV) or a salt thereof is administered wherein R₅ₐ is —CH₃ and R_{5c} is —CH₂OCH₂CH₃.

In one embodiment, a compound of Formula (IV) or a salt thereof is administered wherein R₅ₐ is —CH₃ and R_{5c} is —CH₂CH₂CH₃.

In one embodiment, a compound of Formula (IV) or a salt thereof is administered wherein R₅ₐ is —CH₃ and R_{5c} is —CH₂N₃.

In one embodiment, a compound of Formula (IV) or a salt thereof is administered wherein R₅ₐ is —CH₃ and R_{5c} is —CH₂NH₂.

In one embodiment, a compound of Formula (IV) or a salt thereof is administered wherein R₅ₐ is —CH₃ and R_{5c} is —CH₂NHC(O)OCH₃.

In one embodiment, a compound of Formula (IV) or a salt thereof is administered wherein R₅ₐ is —CH₂OH and R_{5c} is —CH₃.

In one embodiment, a compound of Formula (IV) or a salt thereof is administered wherein R₅ₐ is —CH₂OCH₃ and R_{5c} is —CH₃.

In one embodiment, a compound of Formula (II) or a salt thereof is administered having the structure:

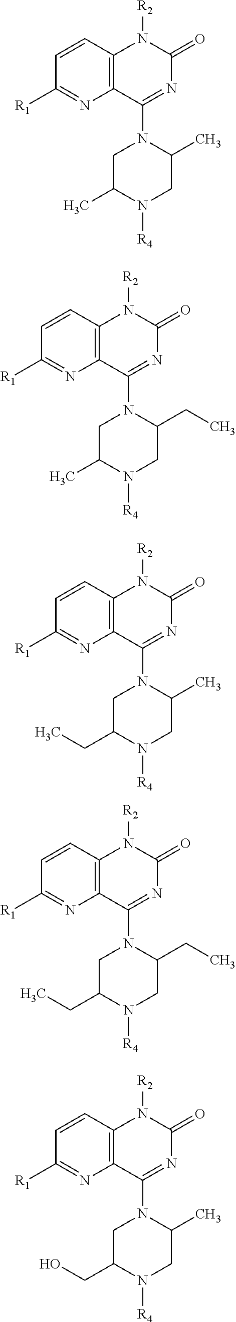

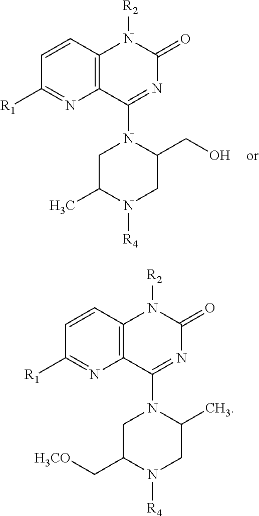

In one embodiment, a compound of Formula (II) or a salt thereof is administered having the structure:

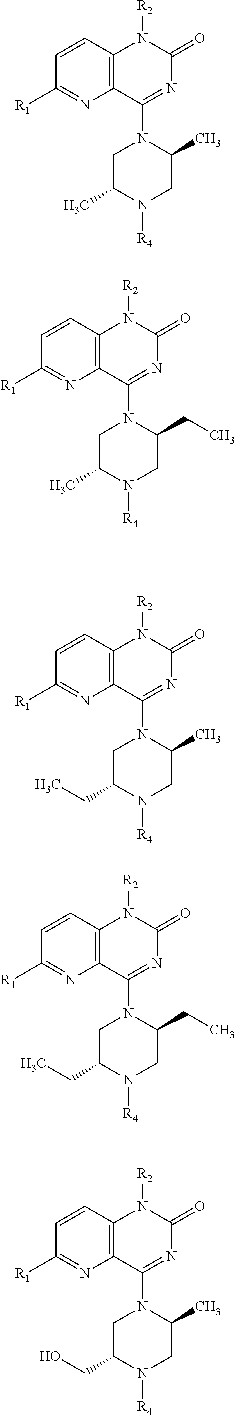

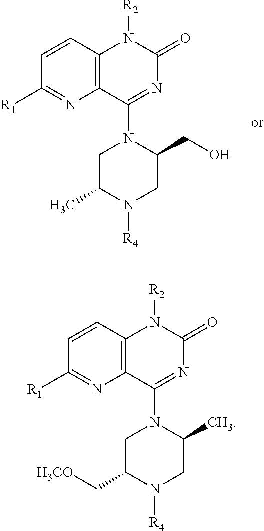

In one embodiment, a compound of Formula (II) or a salt thereof is administered having the structure:

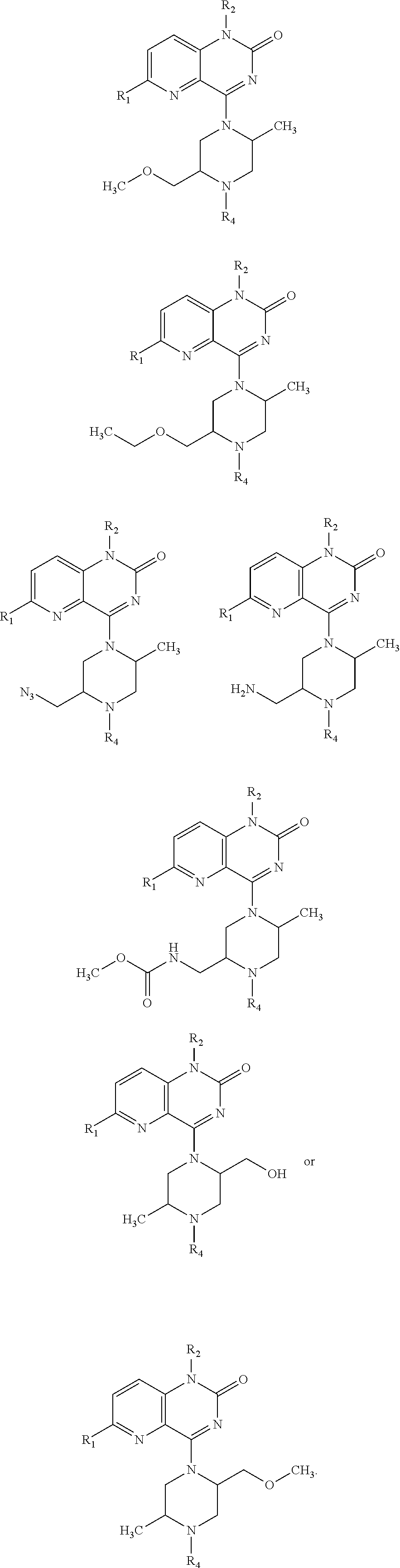

In one embodiment, a compound of Formula (II) or a salt thereof is administered having the structure:

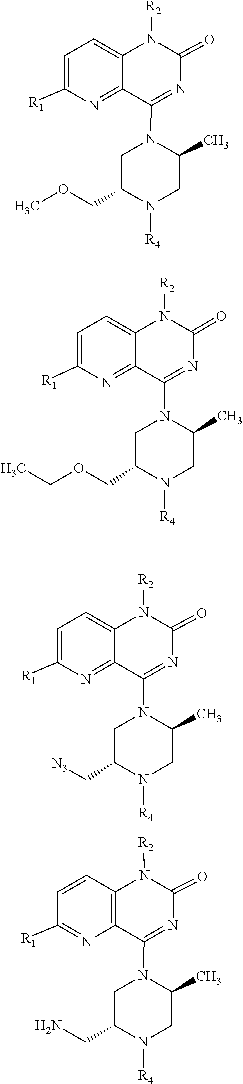

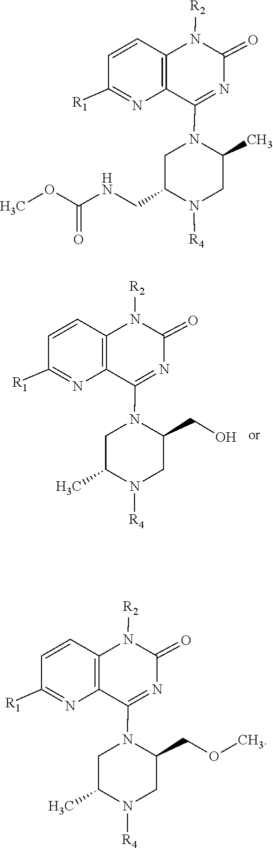

In one embodiment, a compound of Formula (II) or a salt thereof is administered wherein R₄ is:

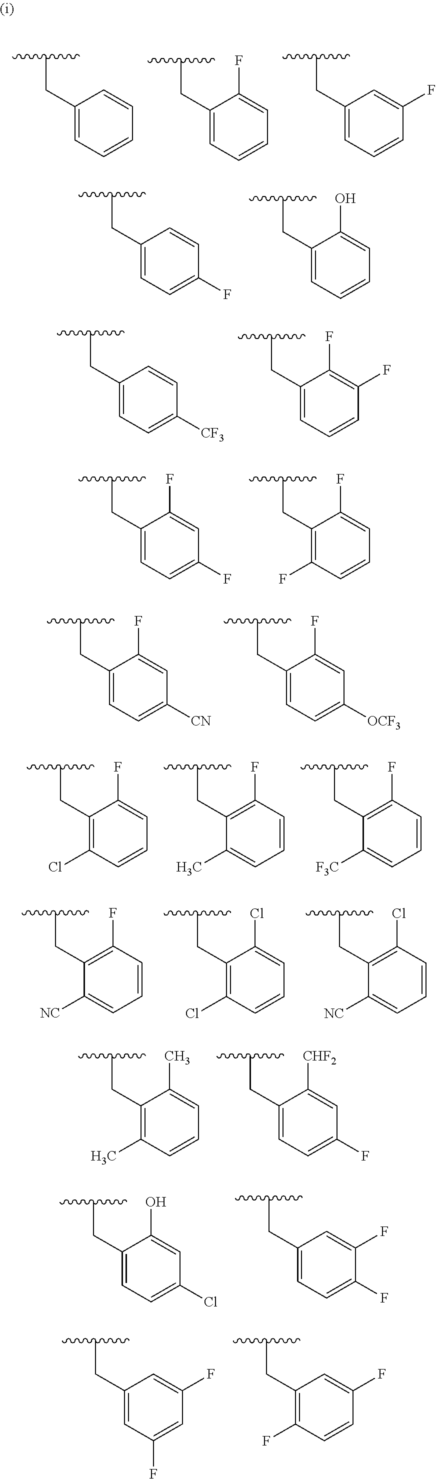

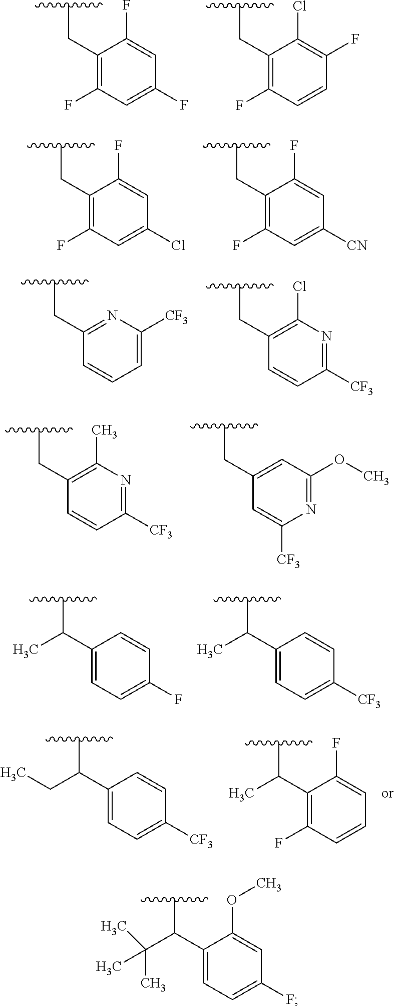

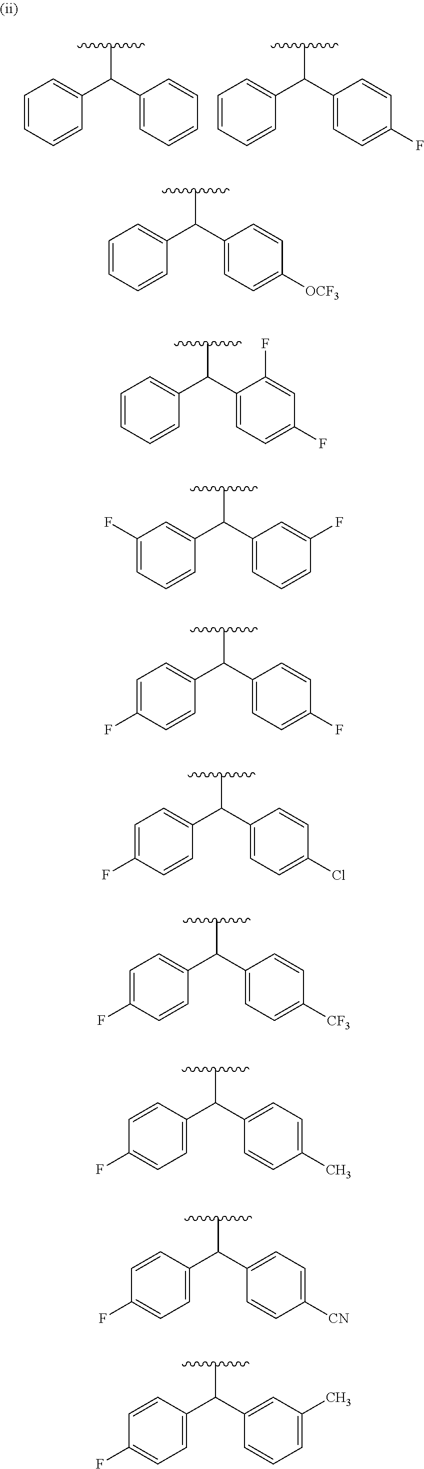

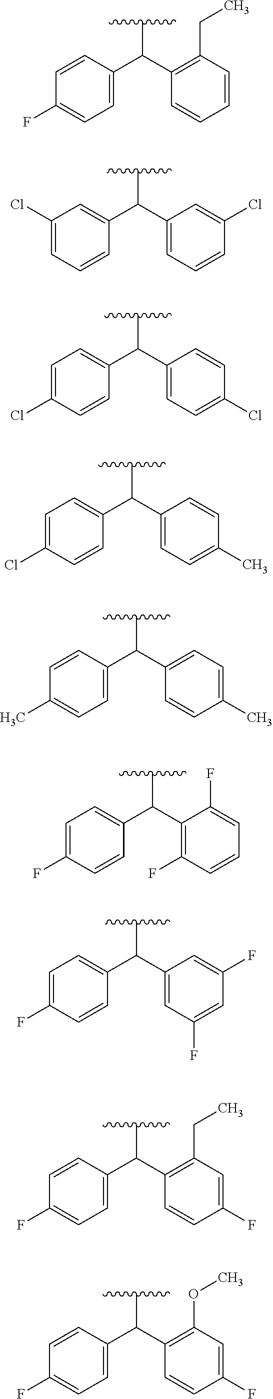

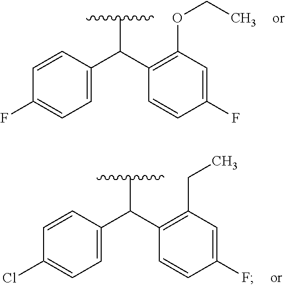 [SMILES: *C(c1ccc(F)cc1)c1ccc(F)cc1OCC ; *C(c1ccc(Cl)cc1)c1ccc(F)cc1CC]

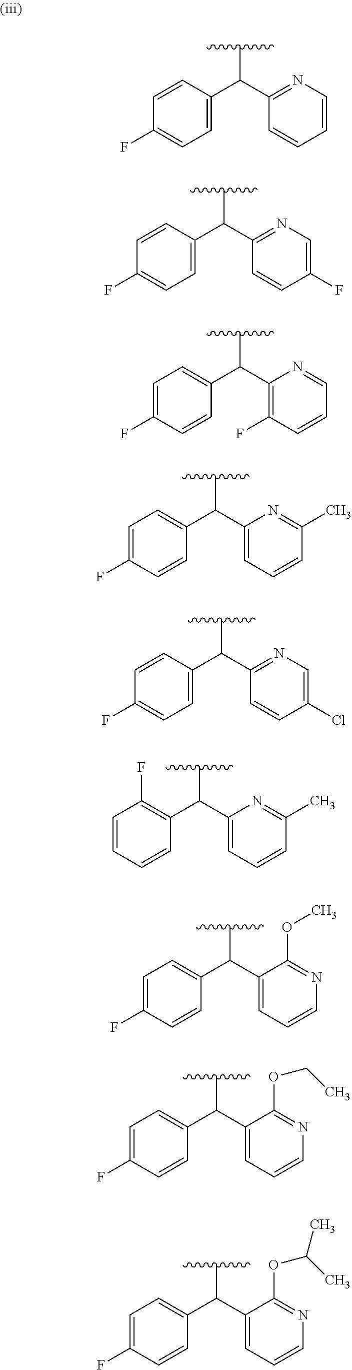

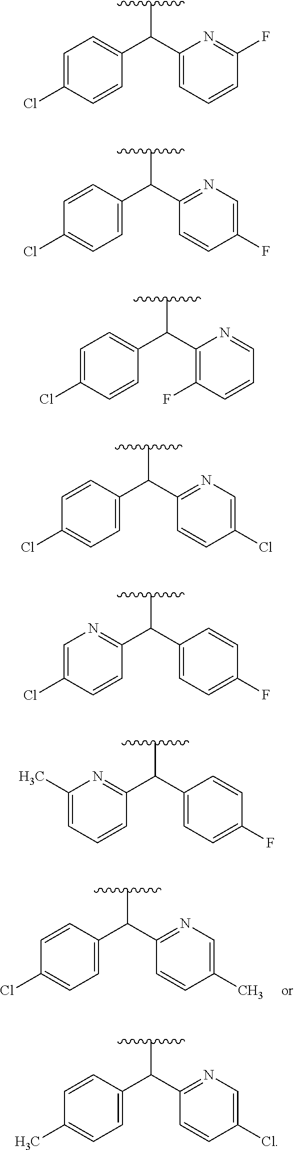

In some embodiments, a compound of Formula (II) or a salt thereof is administered wherein R₁ is H, Br, —CN, or —OCH₃; and R₂ is —CH₃.

In one embodiment, a compound of Formula (II) or a salt thereof is administered wherein R₄ is:

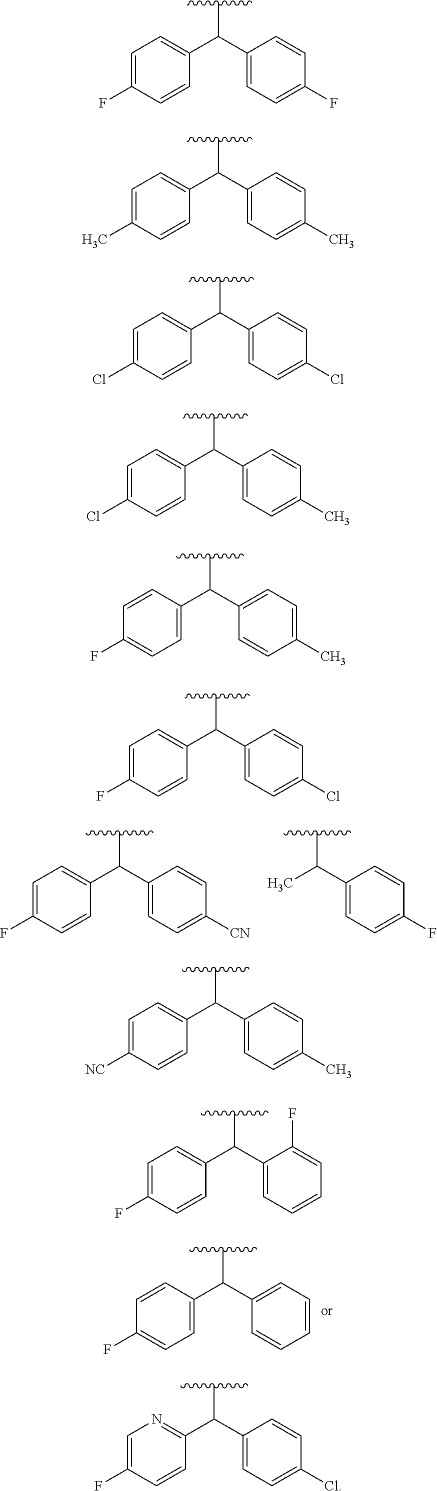

In some embodiments, a compound of Formula (II) or a salt thereof is administered wherein R₁ is H, Br, —CN, or —OCH₃; and R₂ is —CH₃. In some embodiments, a compound of Formula (II) or a salt thereof is administered wherein R₁ is —CN; and R₂ is —CH₃.

In one embodiment, a compound of Formula (II) or a salt thereof is administered wherein R₁ is —CN; and R₂ is —CH₃.

In one embodiment, a compound of Formula (IV) or a salt thereof is administered wherein R₁ is —CN; and R₂ is —CH₃.

In one embodiment, a compound of Formula (IV) or a salt thereof is administered wherein R₁ is —CN; R₂ is —CH₃; R₅ₐ is —CH₃; and R_{5c} is —CH₃.

In one embodiment, a compound of Formula (IV) or a salt thereof is administered wherein R₁ is —CN; R₂ is —CH₃; R₅ₐ is —CH₃; and R_{5c} is —CH₂CH₃.

In one embodiment, a compound of Formula (IV) or a salt thereof is administered wherein R₁ is —CN; R₂ is —CH₃; R₅ₐ is —CH₃; and R_{5c} is —CH₂CH₂CH₃.

In one embodiment, a compound of Formula (IV) or a salt thereof is administered wherein R₁ is —CN; R₂ is —CH₃; R₅ₐ is —CH₂CH₃; and R_{5c} is —CH₂CH₃.

In one embodiment, a compound of Formula (IV) or a salt thereof is administered wherein R₁ is —CN; R₂ is —CH₃; R₅ₐ is —CH₃; and R_{5c} is —CH₃, —CH₂CH₃, or —CH₂CH₂CH₃.

In one embodiment, a compound of Formula (II) or a salt thereof is administered wherein R₁ is —CN; R₂ is —CH₃; R₄ is —CHR₄ₐR_{4b}; and R_{4b} is —CH₃.

In one embodiment, a compound of Formula (II) or a salt thereof is administered wherein R₁ is —CN; R₂ is —CH₃; R₄ is —CHR₄ₐR_{4b}; and R_{4b} is —CH₂CH₃.

In one embodiment, a compound of Formula (II) or a salt thereof is administered wherein R₁ is —CN; R₂ is —CH₃; R₄ is —CHR₄ₐR_{4b}; and R_{4b} is —CH₂CH₂CH₃.

In one embodiment, a compound of Formula (II) or a salt thereof is administered wherein R₁ is —CN; R₂ is —CH₃; R₄ is —CHR₄ₐR_{4b}; and R_{4b} is —CH₃, —CH₂CH₃, or —CH₂CH₂CH₃.

In one embodiment, a compound of Formula (II) or a salt thereof is administered wherein R₄ is —CHR₄ₐR_{4b}; and R₄ₐ is phenyl substituted with 1 to 2 substituents independently selected from F, Cl, —CF₃—OCF₃, or —OCH₂(cyclopropyl). In some embodiments, a compound of Formula (II) or a salt thereof is administered wherein R₁ is —CN; and R₂ is —CH₃.

In one embodiment, a compound of Formula (II) or a salt thereof is administered wherein R₄ is —CHR₄ₐR_{4b}; R₄ₐ is phenyl substituted-CF₃ or —OCF₃; and R_{4b} is —CH₃, —CH₂CH₃, or —CH₂CH₂CH₃. In some embodiments, a compound of Formula (II) or a salt thereof is administered wherein R₁ is —CN; and R₂ is —CH₃.

In one embodiment, a compound of Formula (II) or a salt thereof is administered wherein R₄ is —CHR₄ₐR_{4b}; R₄ₐ is phenyl substituted-CF₃ or —OCF₃; and R_{4b} is —CH₃. In some embodiments, a compound of Formula (II) or a salt thereof is administered wherein R₁ is —CN; and R₂ is —CH₃.

In one embodiment, a compound of Formula (II) or a salt thereof is administered wherein R₄ is —CHR₄ₐR_{4b}; R₄ₐ is phenyl substituted-CF₃ or —OCF₃; and R_{4b} is —CH₂CH₃. In some embodiments, a compound of Formula (II) or a salt thereof is administered wherein R₁ is —CN; and R₂ is —CH₃.

In one embodiment, a compound of Formula (II) or a salt thereof is administered wherein R₄ is —CHR₄ₐR_{4b}; R₄ₐ is phenyl substituted-CF₃ or —OCF₃; and R_{4b} is —CH₂CH₂CH₃. In some embodiments, a compound of Formula (II) or a salt thereof is administered wherein R₁ is —CN; and R₂ is —CH₃.

In one embodiment, a compound of Formula (II) or a salt thereof is administered wherein R₄ is —CHR₄ₐR_{4b}; R₄ₐ is phenyl substituted with 1 to 2 substituents independently selected from F, Cl, —CF₃—OCF₃, or —OCH₂(cyclopropyl); and R_{4b} is —CH₃, —CH₂CH₃, or —CH₂CH₂CH₃. In some embodiments, a compound of Formula (II) or a salt thereof is administered wherein R₁ is —CN; and R₂ is —CH₃.

In one embodiment, a compound of Formula (II) or a salt thereof is administered wherein R₄ is —CHR₄ₐR_{4b}; and R₄ₐ is pyridinyl. In some embodiments, a compound of Formula (II) or a salt thereof is administered wherein R₁ is —CN; and R₂ is —CH₃.

In one embodiment, a compound of Formula (II) or a salt thereof is administered wherein R₄ is —CHR₄ₐR_{4b}; and R₄ₐ is pyridinyl substituted with —CF₃. In some embodiments, a compound of Formula (II) or a salt thereof is administered wherein R₁ is —CN; and R₂ is —CH₃.

In one embodiment, a compound of Formula (II) or a salt thereof is administered wherein R₄ is —CHR₄ₐR_{4b}; R₄ₐ is pyridinyl; and R₄ₐ is phenyl substituted with Cl. In some embodiments, a compound of Formula (II) or a salt thereof is administered wherein R₁ is —CN; and R₂ is —CH₃.

In one embodiment, a compound of Formula (II) or a salt thereof is administered wherein R₄ is —CHR₄ₐR_{4b}; R₄ₐ is pyridinyl substituted with —CF₃; and R_{4b} is phenyl substituted with F. In some embodiments, a compound of Formula (II) or a salt thereof is administered wherein R₁ is —CN; and R₂ is —CH₃.

In one embodiment, a compound of Formula (II) or a salt thereof is administered wherein R₄ is —CHR₄ₐR_{4b}; one of R₄ₐ and R_{4b} is phenyl substituted with F; and the other of R₄ₐ and R_{4b} is oxadiazolyl substituted with cyclopropyl. In some embodiments, a compound of Formula (II) or a salt thereof is administered wherein R₁ is —CN; and R₂ is —CH₃.

In one embodiment, a compound of Formula (II) or a salt thereof is administered wherein said compound is: 4-((2S,5R)-4-((2,2-difluorobenzo[d][1,3]dioxol-5-yl)methyl)-2,5-diethylpiperazin-1-yl)-1-methyl-2-oxo-1,2-dihydropyrido[3,2-d]pyrimidine-6-carbonitrile; 4-((2S,5R)-2,5-diethyl-4-(2-fluoro-4-(trifluoromethoxy)benzyl)piperazin-1-yl)-1-methyl-2-oxo-1,2-dihydropyrido[3,2-d]pyrimidine-6-carbonitrile; 4-((2S,5R)-5-ethyl-2-methyl-4-(4-(trifluoromethoxy)benzyl)piperazin-1-yl)-1-methyl-2-oxo-1,2-dihydropyrido[3,2-d]pyrimidine-6-carbonitrile; 4-((2S,5R)-4-((2,2-difluorobenzo[d][1,3]dioxol-5-yl)methyl)-5-ethyl-2-methylpiperazin-1-yl)-1-methyl-2-oxo-1,2-dihydropyrido[3,2-d]pyrimidine-6-carbonitrile; 4-((2S,5R)-5-ethyl-4-(2-fluoro-4-(trifluoromethoxy)benzyl)-2-methylpiperazin-1-yl)-1-methyl-2-oxo-1,2-dihydropyrido[3,2-d]pyrimidine-6-carbonitrile; 4-((2S,5R)-2,5-dimethyl-4-(3,4,5-trifluorobenzyl)piperazin-1-yl)-1-methyl-2-oxo-1,2-dihydropyrido[3,2-d]pyrimidine-6-carbonitrile; 4-((2S,5R)-4-(3,4-difluorobenzyl)-2,5-dimethylpiperazin-1-yl)-1-methyl-2-oxo-1,2-dihydropyrido[3,2-d]pyrimidine-6-carbonitrile; 4-((2S,5R)-4-(2-chloro-4,5-difluorobenzyl)-2,5-dimethylpiperazin-1-yl)-1-methyl-2-oxo-1,2-dihydropyrido[3,2-d]pyrimidine-6-carbonitrile; 4-((2S,5R)-4-((2,2-difluorobenzo[d][1,3]dioxol-5-yl)methyl)-2,5-dimethylpiperazin-1-yl)-1-methyl-2-oxo-1,2-dihydropyrido[3,2-d]pyrimidine-6-carbonitrile; 4-((2S,5R)-4-(2-chloro-4-fluorobenzyl)-2,5-dimethylpiperazin-1-yl)-1-methyl-2-oxo-1,2-dihydropyrido[3,2-d]pyrimidine-6-carbonitrile; 4-((2S,5R)-4-(4-isopropylbenzyl)-2,5-dimethylpiperazin-1-yl)-1-methyl-2-oxo-1,2-dihydropyrido[3,2-d]pyrimidine-6-carbonitrile; 4-((2S,5R)-4-(4-(cyclopropylmethoxy)benzyl)-2,5-diethylpiperazin-1-yl)-1-methyl-2-oxo-1,2-dihydropyrido[3,2-d]pyrimidine-6-carbonitrile; 4-((2S,5R)-2,5-diethyl-4-(2-fluoro-4-(trifluoromethyl)benzyl)piperazin-1-yl)-1-methyl-2-oxo-1,2-dihydropyrido[3,2-d]pyrimidine-6-carbonitrile; 4-((2S,5R)-2,5-diethyl-4-(4-(trifluoromethyl)benzyl)piperazin-1-yl)-1-methyl-2-oxo-1,2-dihydropyrido[3,2-d]pyrimidine-6-carbonitrile; 4-((2S,5R)-4-(4-cyclopropyl-2-fluorobenzyl)-2,5-diethylpiperazin-1-yl)-1-methyl-2-oxo-1,2-dihydropyrido[3,2-d]pyrimidine-6-carbonitrile; 4-((2S,5R)-4-((4,4-difluorocyclohexyl)methyl)-2,5-diethylpiperazin-1-yl)-1-methyl-2-oxo-1,2-dihydropyrido[3,2-d]pyrimidine-6-carbonitrile; or 4-((2S,5R)-4-(4-ethoxybenzyl)-5-ethyl-2-methylpiperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is: 4-((2S,5R)-2,5-diethyl-4-((4-fluorophenyl)(5-(trifluoromethyl)pyridin-2-yl)methyl)piperazin-1-yl)-1-methyl-2-oxo-1,2-dihydropyrido[3,2-d]pyrimidine-6-carbonitrile; 4-((2S,5R)-2,5-diethyl-4-((4-fluorophenyl) (isoxazol-3-yl)methyl)piperazin-1-yl)-1-methyl-2-oxo-1,2-dihydropyrido[3,2-d]pyrimidine-6-carbonitrile; 4-((2S,5R)-4-((5-cyclopropylisoxazol-3-yl)(4-(trifluoromethoxy)phenyl)methyl)-2,5-diethylpiperazin-1-yl)-1-methyl-2-oxo-1,2-dihydropyrido[3,2-d]pyrimidine-6-carbonitrile; 4-((2S,5R)-5-ethyl-4-((4-fluorophenyl)(5-(trifluoromethyl)pyridin-2-yl)methyl)-2-methylpiperazin-1-yl)-1-methyl-2-oxo-1,2-dihydropyrido[3,2-d]pyrimidine-6-carbonitrile (21-22); 4-((2S,5R)-4-((4-cyclopropylthiazol-2-yl)(4-fluorophenyl)methyl)-5-ethyl-2-methylpiperazin-1-yl)-1-methyl-2-oxo-1,2-dihydropyrido[3,2-d]pyrimidine-6-carbonitrile; 4-((2S,5R)-5-ethyl-4-((4-fluorophenyl)(6-(trifluoromethyl)pyridin-2-yl)methyl)-2-methylpiperazin-1-yl)-1-methyl-2-oxo-1,2-dihydropyrido[3,2-d]pyrimidine-6-carbonitrile; 4-((2S,5R)-5-ethyl-4-((4-fluorophenyl) (isoxazol-3-yl)methyl)-2-methylpiperazin-1-yl)-1-methyl-2-oxo-1,2-dihydropyrido[3,2-d]pyrimidine-6-carbonitrile; 4-((2S,5R)-4-((5-cyclopropylpyridin-2-yl)(4-fluorophenyl)methyl)-5-ethyl-2-methylpiperazin-1-yl)-1-methyl-2-oxo-1,2-dihydropyrido[3,2-d]pyrimidine-6-carbonitrile; 4-((2S,5R)-5-ethyl-4-((4-fluorophenyl) (pyridin-2-yl)methyl)-2-methylpiperazin-1-yl)-1-methyl-2-oxo-1,2-dihydropyrido[3,2-d]pyrimidine-6-carbonitrile; 4-((2S,5R)-5-ethyl-2-methyl-4-(pyridin-2-yl(4-(trifluoromethoxy)phenyl)methyl)piperazin-1-yl)-1-methyl-2-oxo-1,2-dihydropyrido[3,2-d]pyrimidine-6-carbonitrile; 4-((2S,5R)-4-((4-chlorophenyl) (pyridin-2-yl)methyl)-5-ethyl-2-methylpiperazin-1-yl)-1-methyl-2-oxo-1,2-dihydropyrido[3,2-d]pyrimidine-6-carbonitrile (23-24); 4-((2S,5R)-5-ethyl-4-((4-fluorophenyl)(6-(trifluoromethyl)pyridin-3-yl)methyl)-2-methylpiperazin-1-yl)-1-methyl-2-oxo-1,2-dihydropyrido[3,2-d]pyrimidine-6-carbonitrile; 4-((2S,5R)-4-((5-cyclopropylisoxazol-3-yl)(4-(trifluoromethoxy)phenyl)methyl)-5-ethyl-2-methylpiperazin-1-yl)-1-methyl-2-oxo-1,2-dihydropyrido[3,2-d]pyrimidine-6-carbonitrile; 4-((2S,5R)-4-((5-cyclopropylisoxazol-3-yl)(4-fluorophenyl)methyl)-5-ethyl-2-methylpiperazin-1-yl)-1-methyl-2-oxo-1,2-dihydropyrido[3,2-d]pyrimidine-6-carbonitrile; 4-((2S,5R)-4-((2-cyclopropylthiazol-5-yl)(4-fluorophenyl)methyl)-5-ethyl-2-methylpiperazin-1-yl)-1-methyl-2-oxo-1,2-dihydropyrido[3,2-d]pyrimidine-6-carbonitrile; 6-chloro-4-((2S,5R)-4-((3-cyclopropyl-1,2,4-oxadiazol-5-yl)(4-fluorophenyl)methyl)-2,5-dimethylpiperazin-1-yl)-1-methylpyrido[3,2-d]pyrimidin-2(1H)-one; 4-((2S,5R)-4-((3-cyclopropyl-1,2,4-oxadiazol-5-yl)(4-fluorophenyl)methyl)-2,5-dimethylpiperazin-1-yl)-1-methyl-2-oxo-1,2-dihydropyrido[3,2-d]pyrimidine-6-carbonitrile (25-26); 4-((2S,5R)-4-((3-(tert-butyl)-1,2,4-oxadiazol-5-yl)(4-fluorophenyl)methyl)-2,5-dimethylpiperazin-1-yl)-1-methyl-2-oxo-1,2-dihydropyrido[3,2-d]pyrimidine-6-carbonitrile; 4-((2S,5R)-4-((3-cyclopropyl-1,2,4-oxadiazol-5-yl)(4-fluorophenyl)methyl)-2,5-dimethylpiperazin-1-yl)-1-methyl-2-oxo-1,2-dihydropyrido[3,2-d]pyrimidine-6-carbonitrile (25-26); 4-((2S,5R)-4-((3-cyclopropyl-1,2,4-oxadiazol-5-yl)(4-fluorophenyl)methyl)-5-ethyl-2-methylpiperazin-1-yl)-1-methyl-2-oxo-1,2-dihydropyrido[3,2-d]pyrimidine-6-carbonitrile; 4-((2S,5R)-2-ethyl-4-((4-fluorophenyl)(5-(trifluoromethyl)pyridin-2-yl)methyl)-5-methylpiperazin-1-yl)-1-methyl-2-oxo-1,2-dihydropyrido[3,2-d]pyrimidine-6-carbonitrile; 4-((2S,5R)-2-ethyl-4-((4-fluorophenyl)(6-(trifluoromethyl)pyridin-2-yl)methyl)-5-methylpiperazin-1-yl)-1-methyl-2-oxo-1,2-dihydropyrido[3,2-d]pyrimidine-6-carbonitrile; 4-((2S,5R)-4-(bis(4-fluorophenyl)methyl)-2,5-dimethylpiperazin-1-yl)-1-methyl-2-oxo-1,2-dihydropyrido[3,2-d]pyrimidine-6-carbonitrile; 4-((2S,5R)-4-((4-fluorophenyl)(5-(trifluoromethyl)pyridin-2-yl)methyl)-2,5-dimethylpiperazin-1-yl)-1-methyl-2-oxo-1,2-dihydropyrido[3,2-d]pyrimidine-6-carbonitrile (27-28); 4-((2S,5R)-4-((4-cyclopropylthiazol-2-yl)(4-fluorophenyl)methyl)-2,5-dimethylpiperazin-1-yl)-1-methyl-2-oxo-1,2-dihydropyrido[3,2-d]pyrimidine-6-carbonitrile; 4-((2S,5R)-4-((4-fluorophenyl) (isoxazol-3-yl)methyl)-2,5-dimethylpiperazin-1-yl)-1-methyl-2-oxo-1,2-dihydropyrido[3,2-d]pyrimidine-6-carbonitrile; 4-((2S,5R)-4-((5-cyclopropylisoxazol-3-yl)(4-(trifluoromethoxy)phenyl)methyl)-2,5-dimethylpiperazin-1-yl)-1-methyl-2-oxo-1,2-dihydropyrido[3,2-d]pyrimidine-6-carbonitrile; 4-((2S,5R)-4-((4-fluorophenyl)(2-(trifluoromethyl) thiazol-4-yl)methyl)-2,5-dimethylpiperazin-1-yl)-1-methyl-2-oxo-1,2-dihydropyrido[3,2-d]pyrimidine-6-carbonitrile; 4-((2S,5S)-4-((4-fluorophenyl)(5-(trifluoromethyl)pyridin-2-yl)methyl)-5-(methoxymethyl)-2-methylpiperazin-1-yl)-1-methyl-2-oxo-1,2-dihydropyrido[3,2-d]pyrimidine-6-carbonitrile; 6-chloro-4-((2S,5S)-4-((4-fluorophenyl)(5-(trifluoromethyl)pyridin-2-yl)methyl)-5-(hydroxymethyl)-2-methylpiperazin-1-yl)-1-methylpyrido[3,2-d]pyrimidin-2(1H)-one; 4-((2S,5S)-4-((4-fluorophenyl)(5-(trifluoromethyl)pyridin-2-yl)methyl)-5-(hydroxymethyl)-2-methylpiperazin-1-yl)-1-methyl-2-oxo-1,2-dihydropyrido[3,2-d]pyrimidine-6-carbonitrile; 4-((2S,5R)-2,5-diethyl-4-((4-fluorophenyl)(2-(trifluoromethyl) thiazol-4-yl)methyl)piperazin-1-yl)-1-methyl-2-oxo-1,2-dihydropyrido[3,2-d]pyrimidine-6-carbonitrile; 4-((2S,5R)-4-((6-(difluoromethyl)pyridin-2-yl)(4-fluorophenyl)methyl)-2,5-diethylpiperazin-1-yl)-1-methyl-2-oxo-1,2-dihydropyrido[3,2-d]pyrimidine-6-carbonitrile; 4-((2S,5R)-4-((3-bromo-1-methyl-1H-1,2,4-triazol-5-yl)(4-fluorophenyl)methyl)-2,5-diethylpiperazin-1-yl)-1-methyl-2-oxo-1,2-dihydropyrido[3,2-d]pyrimidine-6-carbonitrile; 4-((2S,5R)-4-((3-cyclopropyl-1-methyl-1H-1,2,4-triazol-5-yl)(4-fluorophenyl)methyl)-2,5-diethylpiperazin-1-yl)-1-methyl-2-oxo-1,2-dihydropyrido[3,2-d]pyrimidine-6-carbonitrile; 4-((2S,5R)-5-ethyl-4-((4-fluorophenyl)(2-(trifluoromethyl) thiazol-4-yl)methyl)-2-methylpiperazin-1-yl)-1-methyl-2-oxo-1,2-dihydropyrido[3,2-d]pyrimidine-6-carbonitrile; 4-((2S,5R)-4-((6-(difluoromethyl)pyridin-2-yl)(4-fluorophenyl)methyl)-5-ethyl-2-methylpiperazin-1-yl)-1-methyl-2-oxo-1,2-dihydropyrido[3,2-d]pyrimidine-6-carbonitrile; 6-chloro-4-((2S,5R)-4-((5-cyclopropyl-1,2,4-oxadiazol-3-yl)(4-fluorophenyl)methyl)-2,5-diethylpiperazin-1-yl)-1-methylpyrido[3,2-d]pyrimidin-2(1H)-one; 4-((2S,5R)-4-((5-cyclopropyl-1,2,4-oxadiazol-3-yl)(4-fluorophenyl)methyl)-2,5-diethylpiperazin-1-yl)-1-methyl-2-oxo-1,2-dihydropyrido[3,2-d]pyrimidine-6-carbonitrile; 4-((2S,5R)-4-(bis(4-chlorophenyl)methyl)-2,5-dimethylpiperazin-1-yl)-1-methyl-2-oxo-1,2-dihydropyrido[3,2-d]pyrimidine-6-carbonitrile; 4-((2S,5R)-4-((4-cyanophenyl)(4-fluorophenyl)methyl)-2,5-dimethylpiperazin-1-yl)-1-methyl-2-oxo-1,2-dihydropyrido[3,2-d]pyrimidine-6-carbonitrile; or 4-((2S,5R)-4-((4-fluorophenyl)(3-(trifluoromethyl)bicyclo[1.1.1]pentan-1-yl)methyl)-2,5-dimethylpiperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is: 4-((2S,5R)-4-(1-(4-cyclopropylphenyl)ethyl)-2,5-dimethylpiperazin-1-yl)-1-methyl-2-oxo-1,2-dihydropyrido[3,2-d]pyrimidine-6-carbonitrile; 4-((2S,5R)-2,5-diethyl-4-(1-(4-(trifluoromethyl)phenyl)propyl)piperazin-1-yl)-1-methyl-2-oxo-1,2-dihydropyrido[3,2-d]pyrimidine-6-carbonitrile (17-18); 4-((2S,5R)-5-ethyl-2-methyl-4-(1-(4-(trifluoromethyl)phenyl)ethyl)piperazin-1-yl)-1-methyl-2-oxo-1,2-dihydropyrido[3,2-d]pyrimidine-6-carbonitrile (19-20); 4-((2S,5R)-2,5-diethyl-4-(1-(4-(trifluoromethoxy)phenyl)ethyl)piperazin-1-yl)-1-methyl-2-oxo-1,2-dihydropyrido[3,2-d]pyrimidine-6-carbonitrile; 4-((2S,5R)-2,5-diethyl-4-(1-(2-fluoro-4-methoxyphenyl)propyl)piperazin-1-yl)-1-methyl-2-oxo-1,2-dihydropyrido[3,2-d]pyrimidine-6-carbonitrile; 4-((2S,5R)-4-(1-(2,2-difluorobenzo[d][1,3]dioxol-5-yl)ethyl)-2,5-diethylpiperazin-1-yl)-1-methyl-2-oxo-1,2-dihydropyrido[3,2-d]pyrimidine-6-carbonitrile; 4-((2S,5R)-2,5-diethyl-4-(1-(4-methoxyphenyl)ethyl)piperazin-1-yl)-1-methyl-2-oxo-1,2-dihydropyrido[3,2-d]pyrimidine-6-carbonitrile; 4-((2S,5R)-2,5-diethyl-4-(1-(4-isopropoxyphenyl)ethyl)piperazin-1-yl)-1-methyl-2-oxo-1,2-dihydropyrido[3,2-d]pyrimidine-6-carbonitrile; 4-((2S,5R)-2,5-diethyl-4-(1-(4-(trifluoromethyl)phenyl)ethyl)piperazin-1-yl)-1-methyl-2-oxo-1,2-dihydropyrido[3,2-d]pyrimidine-6-carbonitrile; 4-((2S,5R)-2,5-diethyl-4-(4-(trifluoromethoxy)benzyl)piperazin-1-yl)-1-methyl-2-oxo-1,2-dihydropyrido[3,2-d]pyrimidine-6-carbonitrile; 4-((2S,5R)-4-(1-(4-(cyclopropylmethoxy)phenyl)ethyl)-2,5-diethylpiperazin-1-yl)-1-methyl-2-oxo-1,2-dihydropyrido[3,2-d]pyrimidine-6-carbonitrile; 4-((2S,5R)-2,5-diethyl-4-(1-(2-fluoro-4-(trifluoromethyl)phenyl)ethyl)piperazin-1-yl)-1-methyl-2-oxo-1,2-dihydropyrido[3,2-d]pyrimidine-6-carbonitrile; 4-((2S,5R)-4-(1-(4-cyclopropylphenyl)ethyl)-2,5-diethylpiperazin-1-yl)-1-methyl-2-oxo-1,2-dihydropyrido[3,2-d]pyrimidine-6-carbonitrile; 4-((2S,5R)-2,5-diethyl-4-(1-(3-fluoro-4-(trifluoromethoxy)phenyl)ethyl)piperazin-1-yl)-1-methyl-2-oxo-1,2-dihydropyrido[3,2-d]pyrimidine-6-carbonitrile; 4-((2S,5R)-4-(1-(4-(cyclopropylmethoxy)-2-fluorophenyl)ethyl)-2,5-diethylpiperazin-1-yl)-1-methyl-2-oxo-1,2-dihydropyrido[3,2-d]pyrimidine-6-carbonitrile; 4-((2S,5R)-2,5-diethyl-4-(1-(4-(trifluoromethoxy)phenyl)propyl)piperazin-1-yl)-1-methyl-2-oxo-1,2-dihydropyrido[3,2-d]pyrimidine-6-carbonitrile; 4-((2S,5R)-2,5-diethyl-4-(1-(2-fluoro-4-(trifluoromethoxy)phenyl)ethyl)piperazin-1-yl)-1-methyl-2-oxo-1,2-dihydropyrido[3,2-d]pyrimidine-6-carbonitrile; 4-((2S,5R)-4-(1-(4-cyclopropyl-2-fluorophenyl)ethyl)-2,5-diethylpiperazin-1-yl)-1-methyl-2-oxo-1,2-dihydropyrido[3,2-d]pyrimidine-6-carbonitrile; 4-((2S,5R)-2,5-diethyl-4-(1-(6-(trifluoromethyl)pyridin-3-yl)ethyl)piperazin-1-yl)-1-methyl-2-oxo-1,2-dihydropyrido[3,2-d]pyrimidine-6-carbonitrile; 4-((2S,5R)-5-ethyl-2-methyl-4-(1-(4-(trifluoromethoxy)phenyl)ethyl)piperazin-1-yl)-1-methyl-2-oxo-1,2-dihydropyrido[3,2-d]pyrimidine-6-carbonitrile; 4-((2S,5R)-5-ethyl-4-(1-(4-isopropoxyphenyl)ethyl)-2-methylpiperazin-1-yl)-1-methyl-2-oxo-1,2-dihydropyrido[3,2-d]pyrimidine-6-carbonitrile; 4-((2S,5R)-5-ethyl-4-(1-(4-methoxyphenyl)ethyl)-2-methylpiperazin-1-yl)-1-methyl-2-oxo-1,2-dihydropyrido[3,2-d]pyrimidine-6-carbonitrile; 4-((2S,5R)-5-ethyl-4-(1-(2-fluoro-4-(trifluoromethyl)phenyl)ethyl)-2-methylpiperazin-1-yl)-1-methyl-2-oxo-1,2-dihydropyrido[3,2-d]pyrimidine-6-carbonitrile; 4-((2S,5R)-4-(1-(4-cyanophenyl)ethyl)-5-ethyl-2-methylpiperazin-1-yl)-1-methyl-2-oxo-1,2-dihydropyrido[3,2-d]pyrimidine-6-carbonitrile; 4-((2S,5R)-4-(1-(4-cyclopropylphenyl)ethyl)-5-ethyl-2-methylpiperazin-1-yl)-1-methyl-2-oxo-1,2-dihydropyrido[3,2-d]pyrimidine-6-carbonitrile; 4-((2S,5R)-4-(1-(4-(cyclopropylmethoxy)phenyl)ethyl)-5-ethyl-2-methylpiperazin-1-yl)-1-methyl-2-oxo-1,2-dihydropyrido[3,2-d]pyrimidine-6-carbonitrile; 4-((2S,5R)-4-(1-(4-(cyclopropylmethoxy)-2-fluorophenyl)ethyl)-5-ethyl-2-methylpiperazin-1-yl)-1-methyl-2-oxo-1,2-dihydropyrido[3,2-d]pyrimidine-6-carbonitrile; 4-((2S,5R)-4-(1-(2,2-difluorobenzo[d][1,3]dioxol-5-yl)ethyl)-5-ethyl-2-methylpiperazin-1-yl)-1-methyl-2-oxo-1,2-dihydropyrido[3,2-d]pyrimidine-6-carbonitrile; 4-((2S,5R)-4-(1-(4-(difluoromethoxy)phenyl)propyl)-5-ethyl-2-methylpiperazin-1-yl)-1-methyl-2-oxo-1,2-dihydropyrido[3,2-d]pyrimidine-6-carbonitrile; 4-((2S,5R)-4-(1-(2,2-difluorobenzo[d][1,3]dioxol-5-yl)propyl)-5-ethyl-2-methylpiperazin-1-yl)-1-methyl-2-oxo-1,2-dihydropyrido[3,2-d]pyrimidine-6-carbonitrile; 4-((2S,5R)-5-ethyl-4-(1-(2-fluoro-4-(trifluoromethoxy)phenyl)ethyl)-2-methylpiperazin-1-yl)-1-methyl-2-oxo-1,2-dihydropyrido[3,2-d]pyrimidine-6-carbonitrile; 4-((2S,5R)-5-ethyl-2-methyl-4-(1-(4-(trifluoromethoxy)phenyl)propyl)piperazin-1-yl)-1-methyl-2-oxo-1,2-dihydropyrido[3,2-d]pyrimidine-6-carbonitrile; 4-((2S,5R)-5-ethyl-4-(1-(2-fluoro-4-(trifluoromethoxy)phenyl)propyl)-2-methylpiperazin-1-yl)-1-methyl-2-oxo-1,2-dihydropyrido[3,2-d]pyrimidine-6-carbonitrile; 4-((2S,5R)-5-ethyl-2-methyl-4-(1-(4-(trifluoromethyl)phenyl)propyl)piperazin-1-yl)-1-methyl-2-oxo-1,2-dihydropyrido[3,2-d]pyrimidine-6-carbonitrile; 4-((2S,5R)-4-(1-(4-cyclopropyl-2-fluorophenyl)ethyl)-5-ethyl-2-methylpiperazin-1-yl)-1-methyl-2-oxo-1,2-dihydropyrido[3,2-d]pyrimidine-6-carbonitrile; 4-((2S,5R)-2-ethyl-5-methyl-4-(1-(4-(trifluoromethyl)phenyl)ethyl)piperazin-1-yl)-1-methyl-2-oxo-1,2-dihydropyrido[3,2-d]pyrimidine-6-carbonitrile; 4-((2S,5R)-2-ethyl-4-(1-(2-fluoro-4-(trifluoromethyl)phenyl)ethyl)-5-methylpiperazin-1-yl)-1-methyl-2-oxo-1,2-dihydropyrido[3,2-d]pyrimidine-6-carbonitrile; 4-((2S,5R)-2-ethyl-5-methyl-4-(1-(4-(trifluoromethoxy)phenyl)ethyl)piperazin-1-yl)-1-methyl-2-oxo-1,2-dihydropyrido[3,2-d]pyrimidine-6-carbonitrile; 4-((2S,5R)-4-(1-(4-fluorophenyl)ethyl)-2,5-dimethylpiperazin-1-yl)-1-methyl-2-oxo-1,2-dihydropyrido[3,2-d]pyrimidine-6-carbonitrile; 4-((2S,5R)-2,5-dimethyl-4-(1-(4-(trifluoromethoxy)phenyl)ethyl)piperazin-1-yl)-1-methyl-2-oxo-1,2-dihydropyrido[3,2-d]pyrimidine-6-carbonitrile; 4-((2S,5R)-2,5-dimethyl-4-(1-(3,4,5-trifluorophenyl)ethyl)piperazin-1-yl)-1-methyl-2-oxo-1,2-dihydropyrido[3,2-d]pyrimidine-6-carbonitrile; 4-((2S,5R)-2,5-dimethyl-4-(1-(4-(trifluoromethyl)phenyl)ethyl)piperazin-1-yl)-1-methyl-2-oxo-1,2-dihydropyrido[3,2-d]pyrimidine-6-carbonitrile; 4-((2S,5R)-4-(1-(2-fluoro-4-(trifluoromethoxy)phenyl)ethyl)-2,5-dimethylpiperazin-1-yl)-1-methyl-2-oxo-1,2-dihydropyrido[3,2-d]pyrimidine-6-carbonitrile; 4-((2S,5R)-4-(1-(2,4-difluorophenyl)ethyl)-2,5-dimethylpiperazin-1-yl)-1-methyl-2-oxo-1,2-dihydropyrido[3,2-d]pyrimidine-6-carbonitrile; 4-((2S,5R)-4-(1-(4-chloro-2-fluorophenyl)ethyl)-2,5-dimethylpiperazin-1-yl)-1-methyl-2-oxo-1,2-dihydropyrido[3,2-d]pyrimidine-6-carbonitrile; 4-((2S,5R)-4-(1-(3,4-difluorophenyl)ethyl)-2,5-dimethylpiperazin-1-yl)-1-methyl-2-oxo-1,2-dihydropyrido[3,2-d]pyrimidine-6-carbonitrile; 4-((2S,5R)-4-(1-(2-fluoro-4-(trifluoromethyl)phenyl)ethyl)-2,5-dimethylpiperazin-1-yl)-1-methyl-2-oxo-1,2-dihydropyrido[3,2-d]pyrimidine-6-carbonitrile; 4-((2S,5R)-4-(1-(2-fluoro-4-(trifluoromethoxy)phenyl)propyl)-2,5-dimethylpiperazin-1-yl)-1-methyl-2-oxo-1,2-dihydropyrido[3,2-d]pyrimidine-6-carbonitrile; 4-((2S,5R)-2,5-dimethyl-4-(1-(4-(trifluoromethoxy)phenyl)propyl)piperazin-1-yl)-1-methyl-2-oxo-1,2-dihydropyrido[3,2-d]pyrimidine-6-carbonitrile; 4-((2S,5R)-4-(1-(4-(difluoromethoxy)phenyl)ethyl)-2,5-dimethylpiperazin-1-yl)-1-methyl-2-oxo-1,2-dihydropyrido[3,2-d]pyrimidine-6-carbonitrile; 4-((2S,5R)-4-(1-(3-fluoro-4-(trifluoromethoxy)phenyl)ethyl)-2,5-dimethylpiperazin-1-yl)-1-methyl-2-oxo-1,2-dihydropyrido[3,2-d]pyrimidine-6-carbonitrile; 4-((2S,5R)-4-(1-(2,2-difluorobenzo[d][1,3]dioxol-5-yl)ethyl)-2,5-dimethylpiperazin-1-yl)-1-methyl-2-oxo-1,2-dihydropyrido[3,2-d]pyrimidine-6-carbonitrile; 4-((2S,5R)-4-(1-(4-cyclopropyl-2-fluorophenyl)ethyl)-2,5-dimethylpiperazin-1-yl)-1-methyl-2-oxo-1,2-dihydropyrido[3,2-d]pyrimidine-6-carbonitrile; 6-chloro-4-((2S,5S)-5-(hydroxymethyl)-2-methyl-4-(1-(4-(trifluoromethyl)phenyl)ethyl)piperazin-1-yl)-1-methylpyrido[3,2-d]pyrimidin-2(1H)-one; 6-chloro-4-((2S,5S)-5-(methoxymethyl)-2-methyl-4-(1-(4-(trifluoromethyl)phenyl)ethyl) piperazin-1-yl)-1-methylpyrido[3,2-d]pyrimidin-2(1H)-one; 4-((2S,5S)-5-(methoxymethyl)-2-methyl-4-(1-(4-(trifluoromethyl)phenyl)ethyl)piperazin-1-yl)-1-methyl-2-oxo-1,2-dihydropyrido[3,2-d]pyrimidine-6-carbonitrile; 4-((2S,5S)-5-(methoxymethyl)-2-methyl-4-(1-(4-(trifluoromethoxy)phenyl)ethyl)piperazin-1-yl)-1-methyl-2-oxo-1,2-dihydropyrido[3,2-d]pyrimidine-6-carbonitrile; 4-((2S,5S)-5-(hydroxymethyl)-2-methyl-4-(1-(4-(trifluoromethoxy)phenyl)ethyl)piperazin-1-yl)-1-methyl-2-oxo-1,2-dihydropyrido[3,2-d]pyrimidine-6-carbonitrile; 6-chloro-4-((2S,5R)-2,5-diethyl-4-(1-(4-((1-hydroxy-2-methylpropan-2-yl)oxy)phenyl)ethyl)piperazin-1-yl)-1-methylpyrido[3,2-d]pyrimidin-2(1H)-one; 6-chloro-4-((2S,5R)-2,5-diethyl-4-(1-(4-((1-methoxy-2-methylpropan-2-yl)oxy)phenyl)ethyl)piperazin-1-yl)-1-methylpyrido[3,2-d]pyrimidin-2(1H)-one; 6-chloro-4-((2S,5R)-2,5-diethyl-4-(1-(4-((1-methoxy-2-methylpropan-2-yl)oxy)phenyl)ethyl)piperazin-1-yl)-1-methylpyrido[3,2-d]pyrimidin-2(1H)-one; 4-((2S,5R)-2,5-diethyl-4-(1-(4-(methoxy-d₃)phenyl)ethyl)piperazin-1-yl)-1-methyl-2-oxo-1,2-dihydropyrido[3,2-d]pyrimidine-6-carbonitrile; 4-((2S,5R)-4-(1-(6-cyclopropylpyridin-3-yl)ethyl)-2,5-diethylpiperazin-1-yl)-1-methyl-2-oxo-1,2-dihydropyrido[3,2-d]pyrimidine-6-carbonitrile; 4-((2S,5R)-2,5-diethyl-4-(1-(2-morpholino-4-(trifluoromethyl)phenyl)ethyl)piperazin-1-yl)-1-methyl-2-oxo-1,2-dihydropyrido[3,2-d]pyrimidine-6-carbonitrile; 4-((2S,5R)-4-(1-(4-(2-cyanopropan-2-yl)phenyl)ethyl)-2,5-diethylpiperazin-1-yl)-1-methyl-2-oxo-1,2-dihydropyrido[3,2-d]pyrimidine-6-carbonitrile; 4-((2S,5R)-4-(1-(4-(cyclopropylmethoxy)-2-fluorophenyl)propyl)-2,5-diethylpiperazin-1-yl)-1-methyl-2-oxo-1,2-dihydropyrido[3,2-d]pyrimidine-6-carbonitrile (29-30); 4-((2S,5R)-4-(1-(4-cyclopropylphenyl)propyl)-2,5-diethylpiperazin-1-yl)-1-methyl-2-oxo-1,2-dihydropyrido[3,2-d]pyrimidine-6-carbonitrile; 4-((2S,5R)-2,5-diethyl-4-(1-(3-methyl-2-oxo-2,3-dihydrobenzo[d]oxazol-5-yl)ethyl)piperazin-1-yl)-1-methyl-2-oxo-1,2-dihydropyrido[3,2-d]pyrimidine-6-carbonitrile; 4-((2S,5R)-2,5-diethyl-4-(1-(2-morpholinopyrimidin-5-yl)ethyl)piperazin-1-yl)-1-methyl-2-oxo-1,2-dihydropyrido[3,2-d]pyrimidine-6-carbonitrile; 4-((2S,5R)-2,5-diethyl-4-(1-(4-(methoxy-d₃)phenyl)propyl)piperazin-1-yl)-1-methyl-2-oxo-1,2-dihydropyrido[3,2-d]pyrimidine-6-carbonitrile; 4-((2S,5R)-2,5-diethyl-4-(1-(4-methoxyphenyl)propyl)piperazin-1-yl)-1-methyl-2-oxo-1,2-dihydropyrido[3,2-d]pyrimidine-6-carbonitrile; 4-((2S,5R)-2,5-diethyl-4-(1-(6-methoxypyridin-2-yl)ethyl)piperazin-1-yl)-1-methyl-2-oxo-1,2-dihydropyrido[3,2-d]pyrimidine-6-carbonitrile; 4-((2S,5R)-4-(1-(4-(cyclopropylmethoxy)phenyl)propyl)-2,5-diethylpiperazin-1-yl)-1-methyl-2-oxo-1,2-dihydropyrido[3,2-d]pyrimidine-6-carbonitrile; 4-((2S,5R)-2,5-diethyl-4-(1-(4-(1-methylcyclopropyl)phenyl)ethyl)piperazin-1-yl)-1-methyl-2-oxo-1,2-dihydropyrido[3,2-d]pyrimidine-6-carbonitrile; 4-((2S,5R)-4-(1-(4-cyanophenyl)propyl)-2,5-diethylpiperazin-1-yl)-1-methyl-2-oxo-1,2-dihydropyrido[3,2-d]pyrimidine-6-carbonitrile; 4-((2S,5R)-4-(1-(4-(difluoromethoxy)phenyl)propyl)-2,5-diethylpiperazin-1-yl)-1-methyl-2-oxo-1,2-dihydropyrido[3,2-d]pyrimidine-6-carbonitrile; 4-((2S,5R)-2,5-diethyl-4-(1-(6-(trifluoromethyl)pyridin-3-yl)propyl)piperazin-1-yl)-1-methyl-2-oxo-1,2-dihydropyrido[3,2-d]pyrimidine-6-carbonitrile; 4-((2S,5R)-2,5-diethyl-4-(1-(2-(trifluoromethyl)pyrimidin-5-yl)ethyl)piperazin-1-yl)-1-methyl-2-oxo-1,2-dihydropyrido[3,2-d]pyrimidine-6-carbonitrile; 4-((2S,5R)-2,5-diethyl-4-(1-(6-methylpyridin-3-yl)ethyl)piperazin-1-yl)-1-methyl-2-oxo-1,2-dihydropyrido[3,2-d]pyrimidine-6-carbonitrile; 4-((2S,5R)-2,5-diethyl-4-(1-(4-(2-oxopyrrolidin-1-yl)phenyl)ethyl)piperazin-1-yl)-1-methyl-2-oxo-1,2-dihydropyrido[3,2-d]pyrimidine-6-carbonitrile; 4-((2S,5R)-4-(1-(4-(difluoromethoxy)-2-fluorophenyl)propyl)-2,5-diethylpiperazin-1-yl)-1-methyl-2-oxo-1,2-dihydropyrido[3,2-d]pyrimidine-6-carbonitrile; 4-((2S,5R)-2,5-diethyl-4-(1-(4-isopropoxyphenyl)propyl)piperazin-1-yl)-1-methyl-2-oxo-1,2-dihydropyrido[3,2-d]pyrimidine-6-carbonitrile; 4-((2S,5R)-2,5-diethyl-4-(1-(p-tolyl)propyl) piperazin-1-yl)-1-methyl-2-oxo-1,2-dihydropyrido[3,2-d]pyrimidine-6-carbonitrile; 4-((2S,5R)-4-(1-(4-chloro-2-fluorophenyl)propyl)-2,5-diethylpiperazin-1-yl)-1-methyl-2-oxo-1,2-dihydropyrido[3,2-d]pyrimidine-6-carbonitrile; 4-((2S,5R)-4-(1-(6-(difluoromethoxy)pyridin-2-yl)ethyl)-2,5-diethylpiperazin-1-yl)-1-methyl-2-oxo-1,2-dihydropyrido[3,2-d]pyrimidine-6-carbonitrile; 4-((2S,5R)-2,5-diethyl-4-(1-(4-(trifluoromethyl)phenyl)butyl)piperazin-1-yl)-1-methyl-2-oxo-1,2-dihydropyrido[3,2-d]pyrimidine-6-carbonitrile (31-32); 4-((2S,5R)-2,5-diethyl-4-(1-(6-(trifluoromethoxy)pyridin-2-yl)ethyl)piperazin-1-yl)-1-methyl-2-oxo-1,2-dihydropyrido[3,2-d]pyrimidine-6-carbonitrile; 4-((2S,5R)-2,5-diethyl-4-(1-(4-(2-morpholinopropan-2-yl)phenyl)ethyl)piperazin-1-yl)-1-methyl-2-oxo-1,2-dihydropyrido[3,2-d]pyrimidine-6-carbonitrile; 4-((2S,5R)-2,5-diethyl-4-(1-(4-methoxyphenyl)-2-methylpropyl)piperazin-1-yl)-1-methyl-2-oxo-1,2-dihydropyrido[3,2-d]pyrimidine-6-carbonitrile; 4-((2S,5R)-4-(1-(4-(2-cyanopropan-2-yl)phenyl)propyl)-2,5-diethylpiperazin-1-yl)-1-methyl-2-oxo-1,2-dihydropyrido[3,2-d]pyrimidine-6-carbonitrile; 4-((2S,5R)-4-(1-(2-cyclopropylbenzo[d]oxazol-5-yl)ethyl)-2,5-diethylpiperazin-1-yl)-1-methyl-2-oxo-1,2-dihydropyrido[3,2-d]pyrimidine-6-carbonitrile; 4-((2S,5R)-4-(1-(4-cyclopropoxyphenyl)propyl)-2,5-diethylpiperazin-1-yl)-1-methyl-2-oxo-1,2-dihydropyrido[3,2-d]pyrimidine-6-carbonitrile; ethyl (1S,2S)-2-(4-(1-((2R,5S)-4-(6-cyano-1-methyl-2-oxo-1,2-dihydropyrido[3,2-d]pyrimidin-4-yl)-2,5-diethylpiperazin-1-yl)ethyl) phenoxy)cyclopropane-1-carboxylate; 4-((2S,5R)-2,5-diethyl-4-(1-(4-isopropoxyphenyl)-2-methylpropyl)piperazin-1-yl)-1-methyl-2-oxo-1,2-dihydropyrido[3,2-d]pyrimidine-6-carbonitrile; 4-((2S,5R)-4-(1-(4-(1-cyanocyclopropyl)phenyl)ethyl)-2,5-diethylpiperazin-1-yl)-1-methyl-2-oxo-1,2-dihydropyrido[3,2-d]pyrimidine-6-carbonitrile; methyl 4-(1-((2R,5S)-4-(6-cyano-1-methyl-2-oxo-1,2-dihydropyrido[3,2-d]pyrimidin-4-yl)-2,5-diethylpiperazin-1-yl)ethyl)benzoate; 4-((2S,5R)-2,5-diethyl-4-(1-(4-(morpholinomethyl)phenyl)propyl)piperazin-1-yl)-1-methyl-2-oxo-1,2-dihydropyrido[3,2-d]pyrimidine-6-carbonitrile; 4-((2 S, 5R)-2,5-diethyl-4-(1-(4-(hydroxymethyl)phenyl)ethyl)piperazin-1-yl)-1-methyl-2-oxo-1,2-dihydropyrido[3,2-d]pyrimidine-6-carbonitrile; 4-((2S,5R)-4-(1-(4-(bromomethyl)phenyl)ethyl)-2,5-diethylpiperazin-1-yl)-1-methyl-2-oxo-1,2-dihydropyrido[3,2-d]pyrimidine-6-carbonitrile; 4-((2S,5R)-2,5-diethyl-4-(1-(4-((4-methoxypiperidin-1-yl)methyl)phenyl)ethyl)piperazin-1-yl)-1-methyl-2-oxo-1,2-dihydropyrido[3,2-d]pyrimidine-6-carbonitrile; 4-((2S,5R)-4-(1-(4-((2,2-dimethylmorpholino)methyl)phenyl)ethyl)-2,5-diethylpiperazin-1-yl)-1-methyl-2-oxo-1,2-dihydropyrido[3,2-d]pyrimidine-6-carbonitrile; 4-((2S,5R)-4-(1-(4-((4,4-difluoropiperidin-1-yl)methyl)phenyl)ethyl)-2,5-diethylpiperazin-1-yl)-1-methyl-2-oxo-1,2-dihydropyrido[3,2-d]pyrimidine-6-carbonitrile; 4-((2S,5R)-4-(1-(4-((2-oxa-6-azaspiro[3.3]heptan-6-yl)methyl)phenyl)ethyl)-2,5-diethylpiperazin-1-yl)-1-methyl-2-oxo-1,2-dihydropyrido[3,2-d]pyrimidine-6-carbonitrile; 4-((2S,5R)-2,5-diethyl-4-(1-(4-(piperidin-1-ylmethyl)phenyl)ethyl)piperazin-1-yl)-1-methyl-2-oxo-1,2-dihydropyrido[3,2-d]pyrimidine-6-carbonitrile; 4-((2S,5R)-4-(1-(4-((4-acetylpiperazin-1-yl)methyl)phenyl)ethyl)-2,5-diethylpiperazin-1-yl)-1-methyl-2-oxo-1,2-dihydropyrido[3,2-d]pyrimidine-6-carbonitrile; 4-((2S,5R)-2,5-diethyl-4-(1-(4-((4-hydroxypiperidin-1-yl)methyl)phenyl)ethyl)piperazin-1-yl)-1-methyl-2-oxo-1,2-dihydropyrido[3,2-d]pyrimidine-6-carbonitrile; 4-((2S,5R)-2,5-diethyl-4-(1-(4-((4-methyl-3-oxopiperazin-1-yl)methyl)phenyl)ethyl)piperazin-1-yl)-1-methyl-2-oxo-1,2-dihydropyrido[3,2-d]pyrimidine-6-carbonitrile; 4-((2S,5R)-2,5-diethyl-4-(1-(4-(((R)-3-hydroxypiperidin-1-yl)methyl)phenyl)ethyl)piperazin-1-yl)-1-methyl-2-oxo-1,2-dihydropyrido[3,2-d]pyrimidine-6-carbonitrile; 4-((2S,5R)-4-(1-(4-(((2S,6R)-2,6-dimethylmorpholino)methyl)phenyl)ethyl)-2,5-diethylpiperazin-1-yl)-1-methyl-2-oxo-1,2-dihydropyrido[3,2-d]pyrimidine-6-carbonitrile; 4-((2S,5R)-5-ethyl-2-methyl-4-(1-(3-methyl-2-oxo-2,3-dihydrobenzo[d]oxazol-5-yl)ethyl)piperazin-1-yl)-1-methyl-2-oxo-1,2-dihydropyrido[3,2-d]pyrimidine-6-carbonitrile; 4-((2S,5R)-4-(1-(4-cyclopropylphenyl)propyl)-5-ethyl-2-methylpiperazin-1-yl)-1-methyl-2-oxo-1,2-dihydropyrido[3,2-d]pyrimidine-6-carbonitrile; 4-((2S,5R)-4-(1-(4-cyclopropyl-2-fluorophenyl)propyl)-5-ethyl-2-methylpiperazin-1-yl)-1-methyl-2-oxo-1,2-dihydropyrido[3,2-d]pyrimidine-6-carbonitrile; 4-((2S,5R)-5-ethyl-4-(1-(4-methoxyphenyl)propyl)-2-methylpiperazin-1-yl)-1-methyl-2-oxo-1,2-dihydropyrido[3,2-d]pyrimidine-6-carbonitrile; 4-((2S,5R)-4-(1-(4-ethoxyphenyl)propyl)-5-ethyl-2-methylpiperazin-1-yl)-1-methyl-2-oxo-1,2-dihydropyrido[3,2-d]pyrimidine-6-carbonitrile; 4-((2S,5R)-4-(1-(4-(cyclopropylmethoxy)phenyl)propyl)-5-ethyl-2-methylpiperazin-1-yl)-1-methyl-2-oxo-1,2-dihydropyrido[3,2-d]pyrimidine-6-carbonitrile; 4-((2S,5R)-4-(1-(4-cyclopropoxyphenyl)propyl)-5-ethyl-2-methylpiperazin-1-yl)-1-methyl-2-oxo-1,2-dihydropyrido[3,2-d]pyrimidine-6-carbonitrile; 4-((2S,5R)-5-ethyl-4-(1-(4-methoxyphenyl)-2-methylpropyl)-2-methylpiperazin-1-yl)-1-methyl-2-oxo-1,2-dihydropyrido[3,2-d]pyrimidine-6-carbonitrile; 4-((2S,5R)-4-(1-(4-(2-cyanopropan-2-yl)phenyl)propyl)-5-ethyl-2-methylpiperazin-1-yl)-1-methyl-2-oxo-1,2-dihydropyrido[3,2-d]pyrimidine-6-carbonitrile; 4-((2S,5R)-4-(1-(3,4-difluorophenyl)propyl)-5-ethyl-2-methylpiperazin-1-yl)-1-methyl-2-oxo-1,2-dihydropyrido[3,2-d]pyrimidine-6-carbonitrile; 4-((2S,5R)-4-(1-(4-bromophenyl)propyl)-5-ethyl-2-methylpiperazin-1-yl)-1-methyl-2-oxo-1,2-dihydropyrido[3,2-d]pyrimidine-6-carbonitrile; 4-((2S,5R)-5-ethyl-4-(1-(4-isopropoxyphenyl)-2-methylpropyl)-2-methylpiperazin-1-yl)-1-methyl-2-oxo-1,2-dihydropyrido[3,2-d]pyrimidine-6-carbonitrile; 4-((2S,5R)-4-(1-(4-(1-cyanocyclopropyl)phenyl)ethyl)-5-ethyl-2-methylpiperazin-1-yl)-1-methyl-2-oxo-1,2-dihydropyrido[3,2-d]pyrimidine-6-carbonitrile; 4-((2S,5R)-4-(1-(4-(cyclopropylmethoxy)-2,6-difluorophenyl)propyl)-5-ethyl-2-methylpiperazin-1-yl)-1-methyl-2-oxo-1,2-dihydropyrido[3,2-d]pyrimidine-6-carbonitrile; 4-((2S,5R)-5-ethyl-2-methyl-4-(1-(4-(tetrahydro-2H-pyran-4-yl)phenyl)ethyl)piperazin-1-yl)-1-methyl-2-oxo-1,2-dihydropyrido[3,2-d]pyrimidine-6-carbonitrile; 4-((2S,5R)-4-(1-(4-(1,3-dioxolan-2-yl)phenyl)propyl)-5-ethyl-2-methylpiperazin-1-yl)-1-methyl-2-oxo-1,2-dihydropyrido[3,2-d]pyrimidine-6-carbonitrile; 4-((2S,5R)-5-ethyl-4-(1-(4-isopropoxyphenyl)propyl)-2-methylpiperazin-1-yl)-1-methyl-2-oxo-1,2-dihydropyrido[3,2-d]pyrimidine-6-carbonitrile; 4-((2S,5R)-5-ethyl-2-methyl-4-(1-(4-(3,3,3-trifluoropropoxy)phenyl)propyl)piperazin-1-yl)-1-methyl-2-oxo-1,2-dihydropyrido[3,2-d]pyrimidine-6-carbonitrile; 4-((2S,5R)-5-ethyl-2-methyl-4-(1-(4-((tetrahydro-2H-pyran-4-yl) methoxy)phenyl)propyl)piperazin-1-yl)-1-methyl-2-oxo-1,2-dihydropyrido[3,2-d]pyrimidine-6-carbonitrile; 4-((2S,5R)-4-(1-(4-(2-cyclopropylethoxy)phenyl)propyl)-5-ethyl-2-methylpiperazin-1-yl)-1-methyl-2-oxo-1,2-dihydropyrido[3,2-d]pyrimidine-6-carbonitrile; 4-((2S,5R)-5-ethyl-2-methyl-4-(1-(4-(oxetan-3-ylmethoxy)phenyl)propyl)piperazin-1-yl)-1-methyl-2-oxo-1,2-dihydropyrido[3,2-d]pyrimidine-6-carbonitrile; 4-((2S,5R)-5-ethyl-2-methyl-4-(1-(4-((1-methylazetidin-3-yl) methoxy)phenyl)propyl)piperazin-1-yl)-1-methyl-2-oxo-1,2-dihydropyrido[3,2-d]pyrimidine-6-carbonitrile; 4-((2S,5R)-5-ethyl-2-methyl-4-(1-(4-((1-methylcyclopropyl) methoxy)phenyl)propyl)piperazin-1-yl)-1-methyl-2-oxo-1,2-dihydropyrido[3,2-d]pyrimidine-6-carbonitrile; 4-((2S,5R)-5-ethyl-2-methyl-4-(1-(4-(thiazol-2-ylmethoxy)phenyl)propyl)piperazin-1-yl)-1-methyl-2-oxo-1,2-dihydropyrido[3,2-d]pyrimidine-6-carbonitrile; 4-((2S,5R)-4-(1-(3-bromo-4-(trifluoromethyl)phenyl)ethyl)-5-ethyl-2-methylpiperazin-1-yl)-1-methyl-2-oxo-1,2-dihydropyrido[3,2-d]pyrimidine-6-carbonitrile; 4-((2S,5R)-5-ethyl-2-methyl-4-(1-(3-(morpholinomethyl)-4-(trifluoromethyl)phenyl)ethyl) piperazin-1-yl)-1-methyl-2-oxo-1,2-dihydropyrido[3,2-d]pyrimidine-6-carbonitrile; 4-((2S,5R)-4-(1-(3-((dimethylamino)methyl)-4-(trifluoromethyl)phenyl)ethyl)-5-ethyl-2-methylpiperazin-1-yl)-1-methyl-2-oxo-1,2-dihydropyrido[3,2-d]pyrimidine-6-carbonitrile; 4-((2S,5R)-5-ethyl-2-methyl-4-(1-(3-(piperidin-1-ylmethyl)-4-(trifluoromethyl)phenyl)ethyl) piperazin-1-yl)-1-methyl-2-oxo-1,2-dihydropyrido[3,2-d]pyrimidine-6-carbonitrile; 4-((2S,5R)-4-(1-(3-cyano-4-(trifluoromethyl)phenyl)ethyl)-5-ethyl-2-methylpiperazin-1-yl)-1-methyl-2-oxo-1,2-dihydropyrido[3,2-d]pyrimidine-6-carbonitrile; 4-((2S,5R)-4-(1-(4-(aminomethyl)phenyl)ethyl)-5-ethyl-2-methylpiperazin-1-yl)-1-methyl-2-oxo-1,2-dihydropyrido[3,2-d]pyrimidine-6-carbonitrile; methyl (4-(1-((2R,5S)-4-(6-cyano-1-methyl-2-oxo-1,2-dihydropyrido[3,2-d]pyrimidin-4-yl)-2-ethyl-5-methylpiperazin-1-yl)ethyl)benzyl)carbamate; 4-((2S,5R)-4-(1-(3-cyclopropyl-1,2,4-oxadiazol-5-yl)ethyl)-2,5-diethylpiperazin-1-yl)-1-methyl-2-oxo-1,2-dihydropyrido[3,2-d]pyrimidine-6-carbonitrile; 4-((2S,5R)-4-(1-(3-cyclopropyl-1,2,4-oxadiazol-5-yl)propyl)-2,5-diethylpiperazin-1-yl)-1-methyl-2-oxo-1,2-dihydropyrido[3,2-d]pyrimidine-6-carbonitrile; 4-((2S,5R)-4-(1-(3-cyclopropyl-1,2,4-oxadiazol-5-yl)-2-methylpropyl)-5-ethyl-2-methylpiperazin-1-yl)-1-methyl-2-oxo-1,2-dihydropyrido[3,2-d]pyrimidine-6-carbonitrile; 4-((2S,5R)-4-(1-(5-cyclopropyl-1,3,4-oxadiazol-2-yl)-2-methylpropyl)-5-ethyl-2-methylpiperazin-1-yl)-1-methyl-2-oxo-1,2-dihydropyrido[3,2-d]pyrimidine-6-carbonitrile; 2-((2R,5S)-4-(6-chloro-1-methyl-2-oxo-1,2-dihydropyrido[3,2-d]pyrimidin-4-yl)-2,5-diethylpiperazin-1-yl)-2-(4-fluorophenyl) acetonitrile; 4-((2S,5R)-4-(1-(4-((2-cyanopropan-2-yl)oxy)phenyl)ethyl)-2,5-dimethylpiperazin-1-yl)-1-methyl-2-oxo-1,2-dihydropyrido[3,2-d]pyrimidine-6-carbonitrile; 4-((2S,5R)-4-(1-(4-cyclopropylphenyl)propyl)-2,5-dimethylpiperazin-1-yl)-1-methyl-2-oxo-1,2-dihydropyrido[3,2-d]pyrimidine-6-carbonitrile; 4-((2S,5R)-4-(1-(4-cyclopropyl-2-fluorophenyl)propyl)-2,5-dimethylpiperazin-1-yl)-1-methyl-2-oxo-1,2-dihydropyrido[3,2-d]pyrimidine-6-carbonitrile; 6-chloro-4-((2 S, 5R)-4-(1-(4-(hydroxymethyl)phenyl)ethyl)-2,5-dimethylpiperazin-1-yl)-1-methylpyrido[3,2-d]pyrimidin-2(1H)-one; 4-((2S,5R)-4-(1-(4-(hydroxymethyl)phenyl)ethyl)-2,5-dimethylpiperazin-1-yl)-1-methyl-2-oxo-1,2-dihydropyrido[3,2-d]pyrimidine-6-carbonitrile; 4-((2S,5R)-4-(1-(4-(bromomethyl)phenyl)ethyl)-2,5-dimethylpiperazin-1-yl)-1-methyl-2-oxo-1,2-dihydropyrido[3,2-d]pyrimidine-6-carbonitrile; 4-((2S,5R)-4-(1-(4-((2,2-dimethylmorpholino)methyl)phenyl)ethyl)-2,5-dimethylpiperazin-1-yl)-1-methyl-2-oxo-1,2-dihydropyrido[3,2-d]pyrimidine-6-carbonitrile; 4-((2S,5R)-4-(1-(4-(((1S,4S)-2-oxa-5-azabicyclo[2.2.1]heptan-5-yl)methyl)phenyl)ethyl)-2,5-dimethylpiperazin-1-yl)-1-methyl-2-oxo-1,2-dihydropyrido[3,2-d]pyrimidine-6-carbonitrile; 4-((2S,5R)-4-(1-(4-(((2S,6R)-2,6-dimethylmorpholino)methyl)phenyl)ethyl)-2,5-dimethylpiperazin-1-yl)-1-methyl-2-oxo-1,2-dihydropyrido[3,2-d]pyrimidine-6-carbonitrile; 4-((2S,5R)-4-(1-(4-((4,4-difluoropiperidin-1-yl)methyl)phenyl)ethyl)-2,5-dimethylpiperazin-1-yl)-1-methyl-2-oxo-1,2-dihydropyrido[3,2-d]pyrimidine-6-carbonitrile; 4-((2S,5R)-4-(1-(4-((2-oxa-6-azaspiro[3.3]heptan-6-yl)methyl)phenyl)ethyl)-2,5-dimethylpiperazin-1-yl)-1-methyl-2-oxo-1,2-dihydropyrido[3,2-d]pyrimidine-6-carbonitrile; 6-chloro-1-methyl-4-((2S,5R)-2-methyl-5-propyl-4-(1-(4-(trifluoromethyl)phenyl)ethyl)piperazin-1-yl)pyrido[3,2-d]pyrimidin-2(1H)-one; 1-methyl-4-((2S,5R)-2-methyl-5-propyl-4-(1-(4-(trifluoromethyl)phenyl)ethyl)piperazin-1-yl)-2-oxo-1,2-dihydropyrido[3,2-d]pyrimidine-6-carbonitrile (33-34); 6-chloro-4-((2S,5S)-5-(methoxymethyl)-2-methyl-4-(1-(4-(trifluoromethyl)phenyl)propyl)piperazin-1-yl)-1-methylpyrido[3,2-d]pyrimidin-2(1H)-one; 4-((2S,5S)-5-(methoxymethyl)-2-methyl-4-(1-(4-(trifluoromethyl)phenyl)propyl)piperazin-1-yl)-1-methyl-2-oxo-1,2-dihydropyrido[3,2-d]pyrimidine-6-carbonitrile; 4-((2S,5S)-5-(ethoxymethyl)-2-methyl-4-(1-(4-(trifluoromethyl)phenyl) propyl)piperazin-1-yl)-1-methyl-2-oxo-1,2-dihydropyrido[3,2-d]pyrimidine-6-carbonitrile; 4-((2S,5S)-5-(azidomethyl)-2-methyl-4-(1-(4-(trifluoromethyl)phenyl)ethyl) piperazin-1-yl)-6-chloro-1-methylpyrido[3,2-d]pyrimidin-2(1H)-one; 4-((2S,5R)-5-(aminomethyl)-2-methyl-4-(1-(4-(trifluoromethyl)phenyl)ethyl)piperazin-1-yl)-6-chloro-1-methylpyrido[3,2-d]pyrimidin-2(1H)-one; 4-((2S,5S)-5-(methoxymethyl)-2-methyl-4-(1-(4-(trifluoromethyl)phenyl)ethyl)piperazin-1-yl)-1-methyl-2-oxo-1,2-dihydropyrido[3,2-d]pyrimidine-6-carbonitrile; 4-((2R,5R)-2-(hydroxymethyl)-5-methyl-4-(1-(4-(trifluoromethyl)phenyl)ethyl)piperazin-1-yl)-1-methyl-2-oxo-1,2-dihydropyrido[3,2-d]pyrimidine-6-carbonitrile; 6-chloro-4-((2R,5R)-2-(methoxymethyl)-5-methyl-4-(1-(4-(trifluoromethyl)phenyl)ethyl)piperazin-1-yl)-1-methylpyrido[3,2-d]pyrimidin-2(1H)-one; 4-((2R,5R)-2-(methoxymethyl)-5-methyl-4-(1-(4-(trifluoromethyl)phenyl)ethyl)piperazin-1-yl)-1-methyl-2-oxo-1,2-dihydropyrido[3,2-d]pyrimidine-6-carbonitrile; 4-((2R,5R)-5-ethyl-2-(hydroxymethyl)-4-(1-(4-(trifluoromethyl)phenyl)ethyl)piperazin-1-yl)-1-methyl-2-oxo-1,2-dihydropyrido[3,2-d]pyrimidine-6-carbonitrile; 4-((2S,5R)-5-ethyl-2-methyl-4-(1-(4-(trifluoromethyl)phenyl)ethyl)piperazin-1-yl)-2-oxo-1,2-dihydropyrido[3,2-d]pyrimidine-6-carbonitrile; 4-((2S,5R)-2,5-diethyl-4-(1-(4-(trifluoromethyl)phenyl)propyl)piperazin-1-yl)-2-oxo-1,2-dihydropyrido[3,2-d]pyrimidine-6-carbonitrile; 6-chloro-4-((2S,5R)-5-ethyl-2-methyl-4-(1-(4-(trifluoromethyl)phenyl)ethyl)piperazin-1-yl)-1-(methyl-d₃)pyrido[3,2-d]pyrimidin-2(1H)-one; 4-((2S,5R)-5-ethyl-2-methyl-4-(1-(4-(trifluoromethyl)phenyl)ethyl)piperazin-1-yl)-1-(methyl-d₃)-2-oxo-1,2-dihydropyrido[3,2-d]pyrimidine-6-carbonitrile; 6-chloro-4-((2S,5R)-2,5-diethyl-4-(1-(4-(trifluoromethyl)phenyl)propyl)piperazin-1-yl)-1-methylpyrido[3,2-d]pyrimidin-2(1H)-one; 4-((2S,5R)-2,5-diethyl-4-(1-(4-(trifluoromethyl)phenyl)ethyl)piperazin-1-yl)-6-(hydroxymethyl)-1-methylpyrido[3,2-d]pyrimidin-2(1H)-one; 4-((2 S, 5R)-2,5-diethyl-4-(1-(4-(trifluoromethyl)phenyl)ethyl)piperazin-1-yl)-6-(methoxymethyl)-1-methylpyrido[3,2-d]pyrimidin-2(1H)-one; 6-chloro-4-((2S,5R)-2,5-diethyl-4-(1-(4-(trifluoromethyl)phenyl)ethyl) piperazin-1-yl)-1-methylpyrido[3,2-d]pyrimidin-2(1H)-one; 4-((2S,5R)-2,5-diethyl-4-(1-(4-(trifluoromethyl)phenyl)ethyl)piperazin-1-yl)-6-methoxy-1-methylpyrido[3,2-d]pyrimidin-2(1H)-one; 4-((2S,5R)-2,5-diethyl-4-(1-(4-(trifluoromethyl)phenyl)ethyl)piperazin-1-yl)-6-ethoxy-1-methylpyrido[3,2-d]pyrimidin-2(1H)-one; 4-((2S,5R)-2,5-diethyl-4-(1-(4-(trifluoromethyl)phenyl)ethyl)piperazin-1-yl)-6-(2-(dimethylamino) ethoxy)-1-methylpyrido[3,2-d]pyrimidin-2(1H)-one; 4-((2S,5R)-2,5-diethyl-4-(1-(4-(trifluoromethyl)phenyl)ethyl)piperazin-1-yl)-6-(2-methoxyethoxy)-1-methylpyrido[3,2-d]pyrimidin-2(1H)-one; 4-((2S,5R)-2,5-diethyl-4-(1-(4-(trifluoromethyl)phenyl)propyl)piperazin-1-yl)-6-methoxy-1-methylpyrido[3,2-d]pyrimidin-2(1H)-one; 4-((2S,5R)-2,5-diethyl-4-(1-(4-(trifluoromethyl)phenyl)propyl)piperazin-1-yl)-6-ethoxy-1-methylpyrido[3,2-d]pyrimidin-2(1H)-one; 4-((2S,5R)-2,5-diethyl-4-(1-(4-(trifluoromethyl)phenyl)ethyl)piperazin-1-yl)-6-(difluoromethyl)-1-methylpyrido[3,2-d]pyrimidin-2(1H)-one; 6-(difluoromethyl)-4-((2S,5R)-5-ethyl-2-methyl-4-(1-(4-(trifluoromethyl)phenyl)ethyl)piperazin-1-yl)-1-methylpyrido[3,2-d]pyrimidin-2(1H)-one; 4-((2S,5R)-2,5-diethyl-4-(1-(4-(trifluoromethyl)phenyl)ethyl)piperazin-1-yl)-6-hydroxy-1-methylpyrido[3,2-d]pyrimidin-2(1H)-one; 4-((2 S, 5R)-2,5-diethyl-4-(1-(4-(trifluoromethyl)phenyl)ethyl)piperazin-1-yl)-6-(difluoromethoxy)-1-methylpyrido[3,2-d]pyrimidin-2(1H)-one; 6-chloro-4-((2S,5R)-2,5-dimethyl-4-(1-(3-(trifluoromethyl)bicyclo[1.1.1]pentan-1-yl)propyl)piperazin-1-yl)-1-methylpyrido[3,2-d]pyrimidin-2(1H)-one (diastereomeric mixture); 4-((2S,5R)-2,5-dimethyl-4-(1-(3-(trifluoromethyl)bicyclo[1.1.1]pentan-1-yl)propyl)piperazin-1-yl)-1-methyl-2-oxo-1,2-dihydropyrido[3,2-d]pyrimidine-6-carbonitrile; 4-((2S,5R)-4-(1-cyclopropylpropyl)-2,5-dimethylpiperazin-1-yl)-1-methyl-2-oxo-1,2-dihydropyrido[3,2-d]pyrimidine-6-carbonitrile; 4-((2S,5R)-4-(1-(3,3-difluorocyclobutyl)propyl)-2,5-dimethylpiperazin-1-yl)-1-methyl-2-oxo-1,2-dihydropyrido[3,2-d]pyrimidine-6-carbonitrile; or 4-((2S,5R)-4-(1-(4,4-difluorocyclohexyl)propyl)-2,5-dimethylpiperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is:

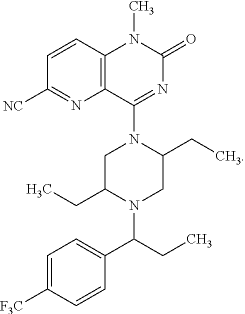 [SMILES: CCC1CN(C(CC)c2ccc(C(F)(F)F)cc2)C(CC)CN1c1nc(=O)n(C)c2ccc(C#N)nc12]

In one embodiment, a compound of Formula (II) or a salt thereof is administered wherein said compound is 4-((2S,5R)-2,5-diethyl-4-(1-(4-(trifluoromethyl)phenyl)propyl) piperazin-1-yl)-1-methyl-2-oxo-1,2-dihydropyrido[3,2-d]pyrimidine-6-carbonitrile (17-18).

In one embodiment, a compound of Formula (II) or a salt thereof is administered wherein said compound is 4-((2S,5R)-2,5-diethyl-4-((S)-1-(4-(trifluoromethyl)phenyl) propyl)piperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is 4-((2S,5R)-2,5-diethyl-4-((R)-1-(4-(trifluoromethyl)phenyl) propyl)piperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is:

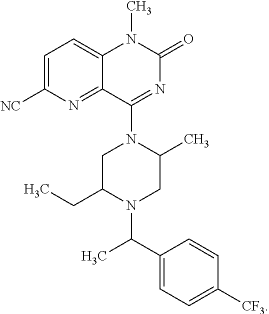 [SMILES: CCC1CN(c2nc(=O)n(C)c3ccc(C#N)nc23)C(C)CN1C(C)c1ccc(C(F)(F)F)cc1]

In one embodiment, a compound of Formula (II) or a salt thereof is administered wherein said compound is 4-((2S,5R)-5-ethyl-2-methyl-4-(1-(4-(trifluoromethyl)phenyl)ethyl) piperazin-1-yl)-1-methyl-2-oxo-1,2-dihydropyrido[3,2-d]pyrimidine-6-carbonitrile (19-20).

In one embodiment, a compound of Formula (II) or a salt thereof is administered wherein said compound is 4-((2S,5R)-5-ethyl-2-methyl-4-((S)-1-(4-(trifluoromethyl)phenyl)ethyl) piperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is 4-((2S,5R)-5-ethyl-2-methyl-4-((R)-1-(4-(trifluoromethyl)phenyl)ethyl) piperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is:

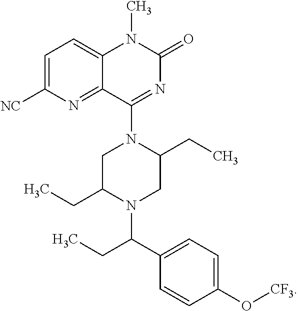 [SMILES: CCC1CN(C(CC)c2ccc(OC(F)(F)F)cc2)C(CC)CN1c1nc(=O)n(C)c2ccc(C#N)nc12]

In one embodiment, a compound of Formula (II) or a salt thereof is administered wherein said compound is 4-((2S,5R)-2,5-diethyl-4-(1-(4-(trifluoromethoxy)phenyl) propyl)piperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is 4-((2S,5R)-2,5-diethyl-4-((S)-1-(4-(trifluoromethoxy)phenyl) propyl)piperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is 4-((2S,5R)-2,5-diethyl-4-((R)-1-(4-(trifluoromethoxy)phenyl) propyl)piperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is:

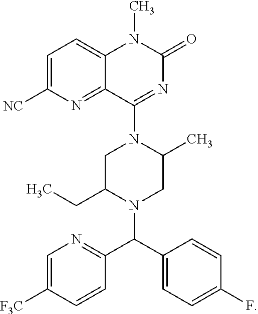 [SMILES: CCC1CN(c2nc(=O)n(C)c3ccc(C#N)nc23)C(C)CN1C(c1ccc(F)cc1)c1ccc(C(F)(F)F)cn1]

In one embodiment, a compound of Formula (II) or a salt thereof is administered wherein said compound is 4-((2S,5R)-5-ethyl-4-((4-fluorophenyl)(5-(trifluoromethyl)pyridin-2-yl)methyl)-2-methylpiperazin-1-yl)-1-methyl-2-oxo-1,2-dihydropyrido[3,2-d]pyrimidine-6-carbonitrile (21-22).

In one embodiment, a compound of Formula (II) or a salt thereof is administered wherein said compound is 4-((2S,5R)-5-ethyl-4-((S)-(4-fluorophenyl)(5-(trifluoromethyl)pyridin-2-yl)methyl)-2-methylpiperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is 4-((2S,5R)-5-ethyl-4-((R)-(4-fluorophenyl)(5-(trifluoromethyl)pyridin-2-yl)methyl)-2-methylpiperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is:

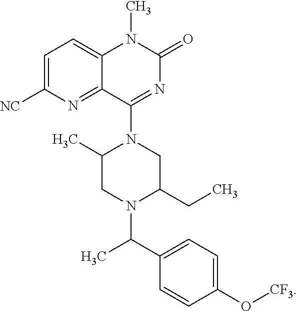 [SMILES: CCC1CN(c2nc(=O)n(C)c3ccc(C#N)nc23)C(C)CN1C(C)c1ccc(OC(F)(F)F)cc1]

In one embodiment, a compound of Formula (II) or a salt thereof is administered wherein said compound is 4-((2S,5R)-5-ethyl-2-methyl-4-(1-(4-(trifluoromethoxy)phenyl)ethyl)piperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is 4-((2S,5R)-5-ethyl-2-methyl-4-((S)-1-(4-(trifluoromethoxy)phenyl)ethyl)piperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is 4-((2S,5R)-5-ethyl-2-methyl-4-((R)-1-(4-(trifluoromethoxy)phenyl)ethyl)piperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is:

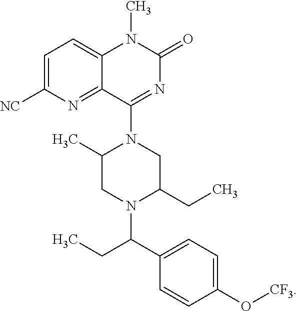 [SMILES: CCC1CN(c2nc(=O)n(C)c3ccc(C#N)nc23)C(C)CN1C(CC)c1ccc(OC(F)(F)F)cc1]

In one embodiment, a compound of Formula (II) or a salt thereof is administered wherein said compound is 4-((2S,5R)-5-ethyl-2-methyl-4-(1-(4-(trifluoromethoxy)phenyl) propyl)piperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is 4-((2S,5R)-5-ethyl-2-methyl-4-((S)-1-(4-(trifluoromethoxy)phenyl) propyl)piperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is 4-((2S,5R)-5-ethyl-2-methyl-4-((R)-1-(4-(trifluoromethoxy)phenyl) propyl)piperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is:

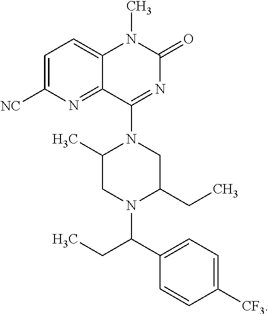 [SMILES: CCC1CN(c2nc(=O)n(C)c3ccc(C#N)nc23)C(C)CN1C(CC)c1ccc(C(F)(F)F)cc1]

In one embodiment, a compound of Formula (II) or a salt thereof is administered wherein said compound is 4-((2S,5R)-5-ethyl-2-methyl-4-(1-(4-(trifluoromethyl)phenyl) propyl)piperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is 4-((2S,5R)-5-ethyl-2-methyl-4-((S)-1-(4-(trifluoromethyl)phenyl) propyl)piperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is 4-((2S,5R)-5-ethyl-2-methyl-4-((R)-1-(4-(trifluoromethyl)phenyl) propyl)piperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is:

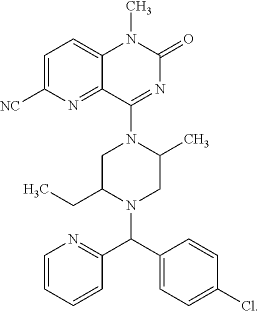 [SMILES: CCC1CN(c2nc(=O)n(C)c3ccc(C#N)nc23)C(C)CN1C(c1ccc(Cl)cc1)c1ccccn1]

In one embodiment, a compound of Formula (II) or a salt thereof is administered wherein said compound is 4-((2S,5R)-4-((4-chlorophenyl) (pyridin-2-yl)methyl)-5-ethyl-2-methylpiperazin-1-yl)-1-methyl-2-oxo-1,2-dihydropyrido[3,2-d]pyrimidine-6-carbonitrile (23-24).

In one embodiment, a compound of Formula (II) or a salt thereof is administered wherein said compound is 4-((2S,5R)-4-((R)-(4-chlorophenyl) (pyridin-2-yl)methyl)-5-ethyl-2-methylpiperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is 4-((2S,5R)-4-((S)-(4-chlorophenyl) (pyridin-2-yl)methyl)-5-ethyl-2-methylpiperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is:

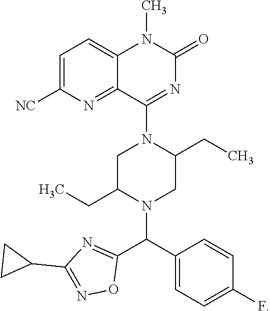 [SMILES: CCC1CN(C(c2ccc(F)cc2)c2nc(C3CC3)no2)C(CC)CN1c1nc(=O)n(C)c2ccc(C#N)nc12]

In one embodiment, a compound of Formula (II) or a salt thereof is administered wherein said compound is 4-((2S,5R)-4-((3-cyclopropyl-1,2,4-oxadiazol-5-yl)(4-fluorophenyl)methyl)-2,5-dimethylpiperazin-1-yl)-1-methyl-2-oxo-1,2-dihydropyrido[3,2-d]pyrimidine-6-carbonitrile (25-26).

In one embodiment, a compound of Formula (II) or a salt thereof is administered wherein said compound is 4-((2S,5R)-4-((R)-(3-cyclopropyl-1,2,4-oxadiazol-5-yl)(4-fluorophenyl)methyl)-2,5-diethylpiperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is 4-((2S,5R)-4-((S)-(3-cyclopropyl-1,2,4-oxadiazol-5-yl)(4-fluorophenyl)methyl)-2,5-diethylpiperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is:

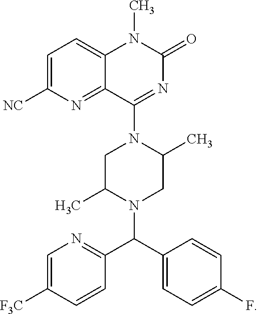 [SMILES: CC1CN(C(c2ccc(F)cc2)c2ccc(C(F)(F)F)cn2)C(C)CN1c1nc(=O)n(C)c2ccc(C#N)nc12]

In one embodiment, a compound of Formula (II) or a salt thereof is administered wherein said compound is 4-((2S,5R)-4-((4-fluorophenyl)(5-(trifluoromethyl)pyridin-2-yl)methyl)-2,5-dimethylpiperazin-1-yl)-1-methyl-2-oxo-1,2-dihydropyrido[3,2-d]pyrimidine-6-carbonitrile (27-28).

In one embodiment, a compound of Formula (II) or a salt thereof is administered wherein said compound is 4-((2S,5R)-4-((S)-(4-fluorophenyl)(5-(trifluoromethyl)pyridin-2-yl)methyl)-2,5-dimethylpiperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is 4-((2S,5R)-4-((R)-(4-fluorophenyl)(5-(trifluoromethyl)pyridin-2-yl)methyl)-2,5-dimethylpiperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is:

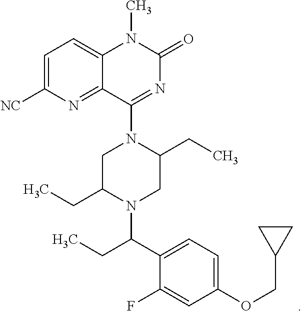 [SMILES: CCC1CN(C(CC)c2ccc(OCC3CC3)cc2F)C(CC)CN1c1nc(=O)n(C)c2ccc(C#N)nc12]

In one embodiment, a compound of Formula (II) or a salt thereof is administered wherein said compound is 4-((2S,5R)-4-(1-(4-(cyclopropylmethoxy)-2-fluorophenyl)propyl)-2,5-diethylpiperazin-1-yl)-1-methyl-2-oxo-1,2-dihydropyrido[3,2-d]pyrimidine-6-carbonitrile (29-30).

In one embodiment, a compound of Formula (II) or a salt thereof is administered wherein said compound is 4-((2S,5R)-4-((S)-1-(4-(cyclopropylmethoxy)-2-fluorophenyl) propyl)-2,5-diethylpiperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is 4-((2S,5R)-4-((R)-1-(4-(cyclopropylmethoxy)-2-fluorophenyl) propyl)-2,5-diethylpiperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is:

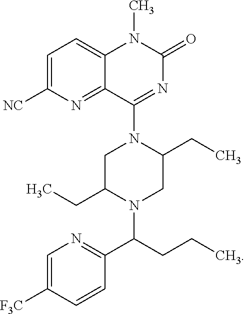 [SMILES: CCCC(c1ccc(C(F)(F)F)cn1)N1CC(CC)N(c2nc(=O)n(C)c3ccc(C#N)nc23)CC1CC]

In one embodiment, a compound of Formula (II) or a salt thereof is administered wherein said compound is 4-((2S,5R)-2,5-diethyl-4-(1-(4-(trifluoromethyl)phenyl)butyl)piperazin-1-yl)-1-methyl-2-oxo-1,2-dihydropyrido[3,2-d]pyrimidine-6-carbonitrile (31-32).

In one embodiment, a compound of Formula (II) or a salt thereof is administered wherein said compound is 4-((2S,5R)-2,5-diethyl-4-((S)-1-(4-(trifluoromethyl)phenyl)butyl)piperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is 4-((2S,5R)-2,5-diethyl-4-((R)-1-(4-(trifluoromethyl)phenyl)butyl)piperazin-1-yl)-1-methyl-2-oxo-1,2-dihydropyrido[3,2-d]pyrimidine-6-carbonitrile.

In one embodiment, a compound of Formula (II) or a salt thereof is administered wherein said compound is:

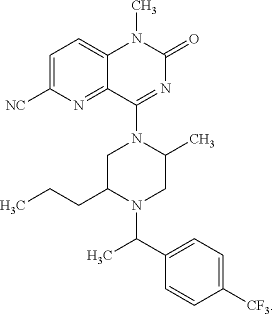 [SMILES: CCCC1CN(c2nc(=O)n(C)c3ccc(C#N)nc23)C(C)CN1C(C)c1ccc(C(F)(F)F)cc1]

In one embodiment, a compound of Formula (II) or a salt thereof is administered wherein said compound is 1-methyl-4-((2S,5R)-2-methyl-5-propyl-4-(1-(4-(trifluoromethyl)phenyl)ethyl)piperazin-1-yl)-2-oxo-1,2-dihydropyrido[3,2-d]pyrimidine-6-carbonitrile (33-34).

In one embodiment, a compound of Formula (II) or a salt thereof is administered wherein said compound is 1-methyl-4-((2S,5R)-2-methyl-5-propyl-4-((S)-1-(4-(trifluoromethyl)phenyl)ethyl)piperazin-1-yl)-2-oxo-1,2-dihydropyrido[3,2-d]pyrimidine-6-carbonitrile.

In one embodiment, a compound of Formula (II) or a salt thereof is administered wherein said compound is 1-methyl-4-((2S,5R)-2-methyl-5-propyl-4-((R)-1-(4-(trifluoromethyl)phenyl)ethyl)piperazin-1-yl)-2-oxo-1,2-dihydropyrido[3,2-d]pyrimidine-6-carbonitrile.

### 2. Compositions and Dosing

Compounds described herein, e.g., in accordance with Formula (I) or (II), such as a compound selected from compounds 1 to 34, and/or pharmaceutically acceptable salts thereof, can be administered by any means suitable for the condition to be treated, which can depend on the need for site-specific treatment or quantity of the compound to be delivered.

Also embraced herein is a class of pharmaceutical compositions comprising a compound, e.g., a compound of Formula (I) or (II), such as a compound selected from compounds 1 to 34, and/or a pharmaceutically acceptable salt thereof; and one or more non-toxic, pharmaceutically-acceptable carriers and/or diluents and/or adjuvants (collectively referred to herein as “carrier” materials) and, if desired, other active ingredients. The compounds, e.g., the compounds of Formula (I) or (II), such as a compound selected from compounds 1 to 34, may be administered by any suitable route, preferably in the form of a pharmaceutical composition adapted to such a route, and in a dose effective for the treatment intended. The compounds and compositions described herein may, for example, be administered orally, mucosally, or parentally including intravascularly, intravenously, intraperitoneally, subcutaneously, intramuscularly, and intrasternally in dosage unit formulations containing conventional pharmaceutically acceptable carriers, adjuvants, and vehicles. For example, the pharmaceutical carrier may contain a mixture of mannitol or lactose and microcrystalline cellulose. The mixture may contain additional components such as a lubricating agent, e.g. magnesium stearate and a disintegrating agent such as crospovidone. The carrier mixture may be filled into a gelatin capsule or compressed as a tablet. The pharmaceutical composition may be administered as an oral dosage form or an infusion, for example.

The dosage regimen for the DGK inhibitor will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration; and the renal and hepatic function of the patient.

In some embodiments, the inhibitor is administered in a therapeutically effective dose. In some embodiments, the therapeutically effective dose provides a dose of the inhibitor that is continuously therapeutically effective for a period of time.

By way of general guidance, the daily oral dosage of each active ingredient, when used for the indicated effects, will range between about 0.001 to about 5000 mg per day, preferably between about 0.01 to about 1000 mg per day, and most preferably between about 0.1 to about 250 mg per day. Intravenously, the most preferred doses will range from about 0.01 to about 10 mg/kg/minute during a constant rate infusion. A DGK inhibitor as described herein may be administered in a single daily dose, or the total daily dosage may be administered in divided doses of two, three, or four times daily.

The compounds are typically administered in admixture with suitable pharmaceutical diluents, excipients, or carriers (collectively referred to herein as pharmaceutical carriers) suitably selected with respect to the intended form of administration, e.g., oral tablets, capsules, elixirs, and syrups, and consistent with conventional pharmaceutical practices.

Dosage forms (pharmaceutical compositions) suitable for administration may contain from about 1 milligram to about 2000 milligrams of active ingredient per dosage unit. In these pharmaceutical compositions the active ingredient will ordinarily be present in an amount of about 0.1-95% by weight based on the total weight of the composition.

A typical capsule for oral administration contains at least one DGK inhibitor (250 mg), lactose (75 mg), and magnesium stearate (15 mg). The mixture is passed through a 60 mesh sieve and packed into a No. L gelatin capsule.

A typical injectable preparation is produced by aseptically placing at least one DGK inhibitor (250 mg) into a vial, aseptically freeze-drying and sealing. For use, the contents of the vial are mixed with 2 mL of physiological saline, to produce an injectable preparation.

For oral administration, a pharmaceutical composition described herein may be in the form of, for example, a tablet, capsule, liquid capsule, suspension, or liquid. The pharmaceutical composition is preferably made in the form of a dosage unit containing a particular amount of the active ingredient. For example, the pharmaceutical composition may be provided as a tablet or capsule comprising an amount of active ingredient in the range of from about 0.1 to 1000 mg, preferably from about 0.25 to 250 mg, and more preferably from about 0.5 to 100 mg. A suitable daily dose for a human or other mammal may vary widely depending on the condition of the patient and other factors, but, can be determined using routine methods.

Regardless of the route of administration selected, the DGK inhibitor for administration as described herein, which may be used in a suitable hydrated form, and/or the pharmaceutical compositions containing same, are formulated into pharmaceutically acceptable dosage forms by conventional methods known to those of skill in the art.

Actual dosage levels of the active ingredients in the pharmaceutical compositions containing the DGK inhibitor may be varied so as to obtain an amount of the active ingredient which is effective to achieve the therapeutic response for a particular patient, composition, and mode of administration, without being toxic to the patient. The selected dosage level will depend upon a variety of factors including the activity of the particular compound employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician having ordinary skill in the art can readily determine and prescribe the effective amount of the pharmaceutical composition required. For example, the physician could start doses of the DGK inhibitor employed in the pharmaceutical composition at levels lower than that required in order to achieve the therapeutic effect and gradually increase the dosage until the effect is achieved.

In general, a suitable daily dose of a DGK inhibitor will be that amount of the compound which is the lowest dose effective to produce a therapeutic effect. Such an effective dose will generally depend upon the factors described above. Generally, oral, intravenous, intracerebroventricular and subcutaneous doses of the DGK inhibitor for a patient will range from about 0.01 to about 50 mg per kilogram of body weight per day. If desired, the effective daily dose of the active compound may be administered as two, three, four, five, six or more sub-doses administered separately at appropriate intervals throughout the day, optionally, in unit dosage forms. In certain aspects, dosing is one administration per day.

Any pharmaceutical composition contemplated herein can, for example, be delivered orally via any acceptable and suitable oral preparations. Exemplary oral preparations, include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition can contain at least one agent selected from sweetening agents, flavoring agents, coloring agents, demulcents, antioxidants, and preserving agents.

A tablet can, for example, be prepared by admixing at least one compound, e.g., a compound of Formula (I) or (II), such as a compound selected from compounds 1 to 34, and/or at least one pharmaceutically acceptable salt thereof, with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents, such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasant tasting drug, or delay disintegration and absorption of the active ingredient in the gastrointestinal tract thereby sustaining the effects of the active ingredient for a longer period. Exemplary water soluble taste masking materials, include, but are not limited to, hydroxypropyl-methylcellulose and hydroxypropyl-cellulose. Exemplary time delay materials, include, but are not limited to, ethyl cellulose and cellulose acetate butyrate.

Hard gelatin capsules can, for example, be prepared by mixing at least one compound, e.g., a compound of Formula (I) or (II), such as a compound selected from compounds 1 to 34, and/or at least one pharmaceutically acceptable salt thereof, with at least one inert solid diluent, such as, for example, calcium carbonate; calcium phosphate; and kaolin.

Soft gelatin capsules can, for example, be prepared by mixing at least one compound, e.g., a compound of Formula (I) or (II), such as a compound selected from compounds 1 to 34 and/or at least one pharmaceutically acceptable salt thereof, with at least one water soluble carrier, such as, for example, polyethylene glycol; and at least one oil medium, such as, for example, peanut oil, liquid paraffin, and olive oil.

An aqueous suspension can be prepared, for example, by admixing at least one compound, e.g., a compound of Formula (I) or (II), such as a compound selected from compounds 1 to 34, and/or at least one pharmaceutically acceptable salt thereof, with at least one excipient suitable for the manufacture of an aqueous suspension. Exemplary excipients suitable for the manufacture of an aqueous suspension, include, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example heptadecaethylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound, e.g., a compound of Formula (I) or (II), such as a compound selected from compounds 1 to 34, and/or at least one pharmaceutically acceptable salt thereof, in either a vegetable oil, such as, for example, arachis oil; olive oil; sesame oil; and coconut oil; or in mineral oil, such as, for example, liquid paraffin. An oily suspension can also contain at least one thickening agent, such as, for example, beeswax; hard paraffin; and cetyl alcohol. In order to provide a palatable oily suspension, at least one of the sweetening agents already described hereinabove, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound, e.g., a compound of Formula (I) or (II), such as a compound selected from compounds 1 to 34, and/or at least one pharmaceutically acceptable salt thereof, with at least one dispersing and/or wetting agent; at least one suspending agent; and/or at least one preservative. Suitable dispersing agents, wetting agents, and suspending agents are as already described above. Exemplary preservatives include, but are not limited to, for example, anti-oxidants, e.g., ascorbic acid. In addition, dispersible powders and granules can also contain at least one excipient, including, but not limited to, for example, sweetening agents; flavoring agents; and coloring agents.

An emulsion of at least one compound, e.g., a compound of Formula (I) or (II), such as a compound selected from compounds 1 to 34, and/or at least one pharmaceutically acceptable salt thereof, can, for example, be prepared as an oil-in-water emulsion. The oily phase of the emulsions comprising compounds of Formula (I) or (II), such as a compound selected from compounds 1 to 34 may be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase may comprise merely an emulsifier, it may comprise a mixture of at least one emulsifier with a fat or an oil or with both a fat and an oil. Suitable emulsifying agents include, but are not limited to, for example, naturally-occurring phosphatides, e.g., soy bean lecithin; esters or partial esters derived from fatty acids and hexitol anhydrides, such as, for example, sorbitan monooleate; and condensation products of partial esters with ethylene oxide, such as, for example, polyoxyethylene sorbitan monooleate. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation for use in the treatment methods include Tween 60, Span 80, cetostearyl alcohol, myristyl alcohol, glyceryl monostearate, sodium lauryl sulfate, glyceryl distearate alone or with a wax, or other materials well known in the art.

The compounds, e.g., those of Formula (I) or (II), such as a compound selected from compounds 1 to 34, and/or at least one pharmaceutically acceptable salt thereof, can, for example, also be delivered intravenously, subcutaneously, and/or intramuscularly via any pharmaceutically acceptable and suitable injectable form. Exemplary injectable forms include, but are not limited to, for example, sterile aqueous solutions comprising acceptable vehicles and solvents, such as, for example, water, Ringer's solution, and isotonic sodium chloride solution; sterile oil-in-water microemulsions; and aqueous or oleaginous suspensions.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (i.e. Captisol), cosolvent solubilization (i.e. propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

A sterile injectable oil-in-water microemulsion can, for example, be prepared by 1) dissolving at least one compound, e.g., a compound of Formula (I) or (II), such as a compound selected from compounds 1 to 34, and/or a pharmaceutically acceptable salt thereof, in an oily phase, such as, for example, a mixture of soybean oil and lecithin; 2) combining a compound, e.g., a compound of Formula (I), and/or a pharmaceutically acceptable salt thereof, containing oil phase with a water and glycerol mixture; and 3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butane diol; and a sterile oleaginous suspension can be prepared with a sterile non-toxic acceptable solvent or suspending medium, such as, for example, sterile fixed oils, e.g., synthetic mono- or diglycerides; and fatty acids, such as, for example, oleic acid.

Pharmaceutically acceptable carriers, adjuvants, and vehicles that may be used in the pharmaceutical compositions include, but are not limited to, ion exchangers, alumina, aluminum stearate, lecithin, self-emulsifying drug delivery systems (SEDDS) such as d-alpha-tocopherol polyethyleneglycol 1000 succinate, surfactants used in pharmaceutical dosage forms such as Tweens, polyethoxylated castor oil such as CREMOPHOR surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutically active compounds described herein can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

The amounts of compounds that are administered and the dosage regimen for treating a disease condition with the compounds and/or compositions described herein depends on a variety of factors, including the age, weight, sex, the medical condition of the subject, the type of disease, the severity of the disease, the route and frequency of administration, and the particular compound employed. Thus, the dosage regimen may vary widely, but can be determined routinely using standard methods. A daily dose of about 0.001 to 100 mg/kg body weight, preferably between about 0.0025 and about 50 mg/kg body weight and most preferably between about 0.005 to 10 mg/kg body weight, may be appropriate. The daily dose can be administered in one to four doses per day. Other dosing schedules include one dose per week and one dose per two day cycle.

For therapeutic purposes, the active compounds described herein are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

Pharmaceutical compositions described herein comprise at least one compound, e.g., a compound of Formula (I), and/or at least one pharmaceutically acceptable salt thereof, and optionally an additional agent selected from any pharmaceutically acceptable carrier, adjuvant, and vehicle. Alternate compositions described herein comprise a compound, such as a compound of the Formula (I) or (II), such as a compound selected from compounds 1 to 34 described herein, or a prodrug thereof, and a pharmaceutically acceptable carrier, adjuvant, or vehicle.

In some embodiments, the administration of the inhibitor of DGKα and/or DGKζ is initiated prior to, subsequently to, during, during the course of, simultaneously, near simultaneously, sequentially, and/or intermittently with the administration of the cell therapy, such as a T cell therapy. In some embodiments, the method involves initiating the administration of the inhibitor of DGKα and/or DGKζ prior to administration of the T cell therapy. In other embodiments, the method involves initiating the administration of the inhibitor of DGKα and/or DGK after administration of the T cell therapy. In some embodiments, the method involves initiating the administration of the inhibitor of DGKα and/or DGKζ on the same day of administration of the T cell therapy (e.g., on Day 1 of the combination therapy). In some embodiments, the method involves initiating the administration of the inhibitor of DGKα and/or DGKζ concurrently with the administration of the T cell therapy. In some embodiments, the administration of the inhibitor is initiated during the administration of the T cell therapy, e.g., the first dose of the inhibitor is administered while the T cell therapy is being infused.

In some embodiments, the inhibitor of DGKα and/or DGKζ is administered in an intermittent dosing regimen. In some embodiments, the intermittent dosing regimen involves non-daily administration of the inhibitor of DGKα and/or DGKζ. In some embodiments, the intermittent dosing regimen involves administration of the inhibitor of DGKα and/or DGKζ in a cycle.

In some embodiments, the inhibitor of DGKα and/or DGKζ is administered in a cycle. In some embodiments, the cycle comprises an administration period in which the inhibitor of DGKα and/or DGKζ is administered followed by a rest period during which the inhibitor of DGKα and/or DGKζ is not administered. In some embodiments, the total number of days of the cycle, e.g., from the beginning of initiating administration of the inhibitor of DGKα and/or DGKζ, is greater than or greater than about or is about 21 days, 28 days, 30 days, 40 days, 50 days, 60 days or more.

In some embodiments, the initiation of the administration of the inhibitor of DGKα and/or DGKζ and the initiation of administration of the T cell therapy are carried out on the same day in at least one cycle, optionally concurrently. In some embodiments, the initiation of the administration of the inhibitor of DGKα and/or DGKζ in at least one cycle is prior to initiation of administration of the T cell therapy. In some embodiments, the initiation of the administration of the inhibitor of DGKα and/or DGKζ in at least one cycle is concurrent with or on the same day as initiation of administration of the T cell therapy. In some embodiments, the method involves initiating the administration of the inhibitor of DGKα and/or DGKζ on the same day of administration of the T cell therapy (e.g., on Day 1 of the combination therapy). In some embodiments, the administration of the inhibitor is initiated on Day 1 of the combination therapy prior to the administration of the T cell therapy.

In some embodiments, the inhibitor of DGKα and/or DGKζ is administered from or from about 0 to 30 days, such as 0 to 15 days, 0 to 6 days, 0 to 96 hours, 0 to 24 hours, 0 to 12 hours, 0 to 6 hours, or 0 to 2 hours, 2 hours to 15 days, 2 hours to 6 days, 2 hours to 96 hours, 2 hours to 24 hours, 2 hours to 12 hours, 2 hours to 6 hours, 6 hours to 30 days, 6 hours to 15 days, 6 hours to 6 days, 6 hours to 96 hours, 6 hours to 24 hours, 6 hours to 12 hours, 12 hours to 30 days, 12 hours to 15 days, 12 hours to 6 days, 12 hours to 96 hours, 12 hours to 24 hours, 24 hours to 30 days, 24 hours to 15 days, 24 hours to 6 days, 24 hours to 96 hours, 96 hours to 30 days, 96 hours to 15 days, 96 hours to 6 days, 6 days to 30 days, 6 days to 15 days, or 15 days to 30 days prior to initiation of the T cell therapy. In some aspects, inhibitor of DGKα and/or DGKζ is administered no more than about 96 hours, 72 hours, 48 hours, 24 hours, 12 hours, 6 hours, 2 hours or 1 hour prior to initiation of the T cell therapy.

In some of any such embodiments in which the inhibitor of DGKα and/or DGKζ is given prior to the cell therapy (e.g. T cell therapy), the administration of the inhibitor of DGKα and/or DGKζ continues at regular intervals until the initiation of the cell therapy (e.g. T cell therapy) and/or for a time after the initiation of the cell therapy (e.g. T cell therapy).

In some embodiments, the inhibitor of DGKα and/or DGKζ is administered, or is further administered, after administration of the cell therapy (e.g. T cell therapy). In some embodiments, the inhibitor of DGKα and/or DGKζ is administered within or within about 1 hours, 2 hours, 6 hours, 12 hours, 24 hours, 48 hours, 96 hours, 4 days, 5 days, 6 days or 7 days, 14 days, 15 days, 21 days, 24 days, 28 days, 30 days, 36 days, 42 days, 60 days, 72 days or 90 days after initiation of administration of the cell therapy (e.g., T cell therapy). In some embodiments, the provided methods involve continued administration, such as at regular intervals, of the inhibitor of DGKα and/or DGKζ after initiation of administration of the cell therapy.

In some embodiments, the inhibitor of DGKα and/or DGKζ is administered up to or up to about 1 day, up to or up to about 2 days, up to or up to about 3 days, up to or up to about 4 days, up to or up to about 5 days, up to or up to about 6 days, up to or up to about 7 days, up to or up to about 12 days, up to or up to about 14 days, up to or up to about 21 days, up to or up to about 24 days, up to or up to about 28 days, up to or up to about 30 days, up to or up to about 35 days, up to or up to about 42 days, up to or up to about 60 days or up to or up to about 90 days, up to or up to about 120 days, up to or up to about 180 days, up to or up to about 240 days, up to or up about 360 days, or up to or up to about 720 days or more after the initiation of administration of the cell therapy (e.g., T cell therapy).

In some embodiments, the administration of the inhibitor is initiated on any of Days 1-6, inclusive, of the combination therapy. In some embodiments, the administration of the inhibitor is initiated on any of Days 1-4, inclusive, of the combination therapy. In some embodiments, the administration of the inhibitor is initiated on Day 1 or Day 2 of the combination therapy. In some embodiments, the administration of the inhibitor is initiated on Day 1 of the combination therapy. In some embodiments, Day 1 of the combination therapy is the day that the T cell therapy is administered.

In some embodiments, the administration of the inhibitor is initiated within or within about 12 hours of the administration of the T cell therapy. In some embodiments, the administration of the inhibitor is initiated within or within about 6 hours of the administration of the T cell therapy. In some embodiments, the administration of the inhibitor is initiated within or within about 4 hours of the administration of the T cell therapy. In some embodiments, the administration of the inhibitor is initiated within or within about 2 hours of the administration of the T cell therapy. In some embodiments, the administration of the inhibitor is initiated within or within about 1 hour of the administration of the T cell therapy.

In some of any such above embodiments, the inhibitor of DGKα and/or DGKζ is administered prior to and after initiation of administration of the cell therapy (e.g. T cell therapy).

In some embodiments, the initiation of the administration of the inhibitor of DGKα and/or DGKζ is carried out at or after, optionally immediately after or within 1 to 3 days after peak or maximum level of the cells of the T cell therapy are detectable in the blood of the subject. In some embodiments, the initiation of the administration of the inhibitor of DGKα and/or DGKζ is carried out at or after, optionally immediately after or within 1 to 3 days after the number of cells of the T cell therapy detectable in the blood, after having been detectable in the blood, is not detectable or is reduced, optionally reduced compared to a preceding time point after administration of the T cell therapy. In some embodiments, the initiation of the administration of the inhibitor of DGKα and/or DGKζ is carried out at or after, optionally immediately after or within 1 to 3 days after the number of cells of the T cell therapy detectable in the blood is decreased by or more than 1.5-fold, 2.0-fold, 3.0-fold, 4.0-fold, 5.0-fold, 10-fold or more the peak or maximum number cells of the T cell therapy detectable in the blood of the subject after initiation of administration of the T cell therapy. In some embodiments, the initiation of the administration of the inhibitor of DGKα and/or DGKζ is carried out at or after, optionally immediately after or within 1 to 3 days after at a time after a peak or maximum level of the cells of the T cell therapy are detectable in the blood of the subject, the number of cells of or derived from the T cells detectable in the blood from the subject is less than less than 10%, less than 5%, less than 1% or less than 0.1% of total peripheral blood mononuclear cells (PBMCs) in the blood of the subject. In some embodiments, the initiation of the administration of the inhibitor of DGKα and/or DGKζ is carried out at or after, optionally immediately after or within 1 to 3 days after the number of cells of the T cell therapy detectable in the blood that are exhausted is increased, e.g., by or more than 1.5-fold, 2.0-fold, 3.0-fold, 4.0-fold, 5.0-fold, 10-fold or more the peak or maximum number cells of the T cell therapy detectable in the blood of the subject that are not exhausted, after initiation of administration of the T cell therapy. In some embodiments, the initiation of the administration of the inhibitor of DGKα and/or DGKζ is carried out at or after, optionally immediately after or within 1 to 3 days after the subject exhibits disease progression and/or has relapsed following remission after treatment with the T cell therapy. In some embodiments, the initiation of the administration of the inhibitor of DGKα and/or DGKζ is carried out at or after, optionally immediately after or within 1 to 3 days after the subject exhibits increased tumor burden as compared to tumor burden at a time prior to or after administration of the T cells and prior to initiation of administration of the inhibitor of DGKα and/or DGKζ.

In some embodiments, the initiation of the administration of the inhibitor of DGKα and/or DGKζ in at least one cycle is after initiation of administration of the T cell therapy. In some embodiments, the initiation of the administration of the inhibitor of DGKα and/or DGKζ is at least or about at least 1 day, at least or about at least 2 days, at least or about at least 3 days, at least or about at least 4 days, at least or about at least 5 days, at least or about at least 6 days, at least or about at least 7 days, at least or about at least 8 days, at least or about at least 9 days, at least or about at least 10 days, at least or at least about 12 days, at least or about at least 14 days, at least or at least about 15 days, at least or about at least 21 days, at least or at least about 24 days, at least or about at least 28 days, at least or about at least 30 days, at least or about at least 35 days or at least or about at least 42 days, at least or about at least 60 days, or at least or about at least 90 days after initiation of the administration of the T cell therapy. In some embodiments, the initiation of the administration of the inhibitor of DGKα and/or DGKζ is carried out at least 2 days after, at least 1 week after, at least 2 weeks after, at least 3 weeks after, or at least 4 weeks after, the initiation of the administration of the T cell therapy. In some embodiments, the initiation of administration of the inhibitor of DGKα and/or DGKζ is carried out 2 to 35 days after the initiation of administration of the T cell therapy. In some embodiments, the initiation of administration of the inhibitor of DGKα and/or DGKζ is carried out 2 to 28 days after the initiation of administration of the T cell therapy. In some embodiments, the initiation of administration of the inhibitor of DGKα and/or DGKζ is carried out 2 to 21 days after the initiation of administration of the T cell therapy. In some embodiments, the initiation of administration of the inhibitor of DGKα and/or DGKζ is carried out 2 to 14 days after the initiation of administration of the T cell therapy. In some embodiments, the initiation of administration of the inhibitor of DGKα and/or DGKζ is carried out 7 to 21 days after the initiation of administration of the T cell therapy. In some embodiments, the initiation of administration of the inhibitor of DGKα and/or DGKζ is carried out 7 to 14 days after the initiation of administration of the T cell therapy. In some embodiments, the initiation of the administration of the inhibitor of DGKα and/or DGKζ is carried out at a time that is greater than or greater than about 14 days, 15 days, 16 days, 17 days, 18 days, 19, days, 20 days, 21 days, 24 days, or 28 days after initiation of the administration of the T cell therapy.

In some embodiments, the inhibitor of DGKα and/or DGKζ is administered several times a day, twice a day, daily, every other day, three times a week, twice a week, or once a week after initiation of the cell therapy. In some embodiments, the inhibitor of DGKα and/or DGKζ is administered every three days. In some embodiments, the inhibitor of DGKα and/or DGKζ is administered every two days. In some embodiments, the inhibitor of DGKα and/or DGKζ is administered daily, such as in a continuous dosing regimen. In some embodiments the inhibitor of DGKα and/or DGKζ is administered twice a day. In some embodiments, the inhibitor of DGKα and/or DGKζ is administered three times a day. In other embodiments, the inhibitor of DGKα and/or DGKζ is administered every other day. In some embodiments, inhibitor of DGKα and/or DGKζ is administered during the administration period for a plurality of consecutive days, such as for up to about 7, 8, 9, 10, 11, 12, 13, 14, 15, 16, 17, 18, 19, 20, 21, 22, 23, 24, 25, 26, 27, 28, 29, 30, or more than 30 consecutive days. In some embodiments, the inhibitor of DGKα and/or DGKζ is administered for greater than or greater than about 7 consecutive days, greater than or greater than about 14 consecutive days, greater than or greater than about 21 consecutive days, greater than or greater than about 21 consecutive days, or greater than or greater than about 28 consecutive days. In some embodiments, the inhibitor of DGKα and/or DGKζ is administered during the administration period for up to 21 consecutive days. In some embodiments, the inhibitor of DGKα and/or DGKζ is administered during the administration period for up to 21 consecutive days, wherein the cycle comprises greater than 30 days beginning upon initiation of the administration of the inhibitor of DGKα and/or DGKζ.

In some embodiments, the inhibitor of DGKα and/or DGKζ is administered at least until the number of engineered T cells of the T cell therapy has peaked in the subject following administration of the T cell therapy. In some embodiments, the inhibitor of DGKα and/or DGKζ is administered until the number of engineered T cells of the T cell therapy has peaked in the subject following administration of the T cell therapy.

In some embodiments, the inhibitor of DGKα and/or DGKζ, optionally over a plurality of cycles, is administered over a period of time between or between about 21 and 42 days, inclusive. In some embodiments, the inhibitor, optionally over a plurality of cycles, is administered over a period of time between or between about 21 and 35 days, inclusive. In some embodiments, the inhibitor, optionally over a plurality of cycles, is administered over a period of time between or between about 21 and 28 days, inclusive. In some embodiments, the inhibitor, optionally over a plurality of cycles, is administered over a period of time between or between about 28 and 42 days, inclusive. In some embodiments, the inhibitor, optionally over a plurality of cycles, is administered over a period of time between or between about 28 and 35 days, inclusive. In some embodiments, the inhibitor, optionally over a plurality of cycles, is administered over a period of time that is or is about 28 days. In some embodiments, the inhibitor, optionally over a plurality of cycles, is administered over a period of time that is or is about 30 days.

In some embodiments, the inhibitor of DGKα and/or DGKζ is administered during the administration period for no more than about 7, 8, 9, 10, 11, 12, 13, 14, 15, 16, 17, 18, 19, 20, 21, 22, 23, 24, 25, 26, 27, 28, 29, 30, or no more than 30 consecutive days. In certain embodiments, the inhibitor of DGKα and/or DGKζ is administered once daily for 14 days over a 21 day treatment cycle. In certain embodiments, the inhibitor of DGKα and/or DGKζ is administered once daily for 21 days over a 28 day treatment cycle. In some embodiments, the inhibitor of DGKα and/or DGKζ is administered during the administration period for no more than 14 consecutive days.

In some embodiments, the inhibitor of DGKα and/or DGKζ is administered in a cycle, wherein the cycle comprises the administration of the inhibitor of DGKα and/or DGKζ for a plurality of consecutive days followed by a rest period during which the inhibitor of DGKα and/or DGKζ is not administered. In some embodiments, the rest period is greater than about 1 day, greater than about 3 consecutive days, greater than about 5 consecutive days, greater than about 7 consecutive days, greater than about 8 consecutive days, greater than about 9 consecutive days, greater than about 10 consecutive days, greater than about 11 consecutive days, greater than about 12 consecutive days, greater than about 13 consecutive days, greater than about 14 consecutive days, greater than about 15 consecutive days, greater than about 16 consecutive days, greater than about 17 consecutive days, greater than about 18 consecutive days, greater than about 19 consecutive days, greater than about 20 consecutive days, or greater than about 21 or more consecutive days. In some embodiments, the rest period is greater than 7 consecutive days, greater than 14 consecutive days, greater than 21 days, or greater than 28 days. In some embodiments, the rest period is greater than about 14 consecutive days. In some embodiments, the cycle of administration of the inhibitor of DGKα and/or DGKζ does not contain a rest period.

In some embodiments, the inhibitor of DGKα and/or DGKζ is administered in a cycle, wherein the cycle is repeated at least one time. In some embodiments, the inhibitor of DGKα and/or DGKζ is administered for at least 2 cycles, at least 3 cycles, at least 4 cycles, at least 5 cycles, at least 6 cycles, at least 7 cycles, at least 8 cycles, at least 9 cycles, at least 10 cycles, at least 11 cycles, or at least 12 cycles. In some embodiments, the inhibitor of DGKα and/or DGKζ is administered for 1, 2, 3, 4, 5, 6, 7, 8, 9, 10, 11, 12, 13, 14, 15, 16, 17, 18, 19, 20, 21, 22, 23, or 24 cycles.

In some embodiments, the inhibitor of DGKα and/or DGKζ is administered six times daily, five times daily, four times daily, three times daily, twice daily, once daily, every other day, every three days, twice weekly, once weekly or only one time prior to or subsequently to initiation of administration of the T cell therapy. In some embodiments, the inhibitor of DGKα and/or DGKζ is administered once on each of the days that the inhibitor is administered. In some embodiments, the inhibitor of DGKα and/or DGKζ is administered in multiple doses in regular intervals prior to, during, during the course of, and/or after the period of administration of the T cell therapy. In some embodiments, the inhibitor of DGKα and/or DGKζ is administered in one or more doses in regular intervals prior to the administration of the T cell therapy. In some embodiments, the inhibitor of DGKα and/or DGKζ is administered in one or more doses in regular intervals after the administration of the T cell therapy. In some embodiments, one or more of the doses of the inhibitor of DGKα and/or DGKζ can occur simultaneously with the administration of a dose of the T cell therapy.

In some embodiments, the method involves administering the T cell therapy to a subject that has been previously administered a therapeutically effective amount of the inhibitor of DGKα and/or DGKζ. In some embodiments, the inhibitor of DGKα and/or DGKζ is administered to a subject before administering a dose of cells expressing a recombinant receptor to the subject. In some embodiments, the treatment with the inhibitor of DGKα and/or DGKζ occurs at the same time as the administration of the dose of cells. In some embodiments, the inhibitor of DGKα and/or DGKζ is administered after the administration of the dose of cells.

In some embodiments, the inhibitor of DGKα and/or DGKζ is administered daily for 7, 8, 9, 10, 11, 12, 13, 14, 15, 16, 17, 18, 19, 20, 21, or more than 21 days. In some embodiments, the inhibitor of DGKα and/or DGKζ is administered twice a day for 7, 8, 9, 10, 11, 12, 13, 14, 15, 16, 17, 18, 19, 20, 21, or more than 21 days. In some embodiments, the inhibitor of DGKα and/or DGKζ is administered three times a day for 7, 8, 9, 10, 11, 12, 13, 14, 15, 16, 17, 18, 19, 20, 21, or more than 21 days. In some embodiments, the inhibitor of DGKα and/or DGKζ (is administered every other day for 7, 8, 9, 10, 11, 12, 13, 14, 15, 16, 17, 18, 19, 20, 21, or more than 21 days.

In some embodiments of the methods provided herein, the inhibitor of DGKα and/or DGKζ and the T cell therapy are administered simultaneously or near simultaneously. In certain embodiments, a patient in need of treatment is administered an inhibitor of DGKα and/or DGKζ at a time, for a duration and at amount therapeutically sufficient to at least partially reverse the state of exhaustion of the T cells of the T cell therapy.

### C. Administration of a Checkpoint Inhibitor Antagonist

In some of any of the preceding embodiments, the combination therapy (e.g., that including the administration of an inhibitor of DGKα and/or DGKζ and the administration of a T cell therapy) further includes the administration of a checkpoint antagonist (also referred to as a checkpoint inhibitor) to the subject having the disease or condition. In some embodiments, the checkpoint antagonist is administered before (prior to), concurrently with, or after (subsequently or subsequent to) the administration of the inhibitor of DGKα and/or DGKζ. In some embodiments, the checkpoint antagonist is administered before (prior to), concurrently with, or after (subsequently or subsequent to) the administration of the T cell therapy.

In some embodiments, the combination therapy includes the administration of an inhibitor of DGKα and/or DGKζ, the administration of a T cell therapy, and the administration of an antagonist of a checkpoint inhibitor. In some embodiments, the combination therapy includes the administration of an inhibitor of DGKα and/or DGKζ, the administration of a T cell therapy, and the administration of two or more antagonists of one or more checkpoint inhibitors.

In some embodiments, the checkpoint antagonist is an antagonist or inhibitor of the PD1/PD-L1 axis. In some embodiments, the checkpoint antagonist is an antagonist or an inhibitor of CTLA-4. In some embodiments, two or more antagonists may be administered in which one is an antagonist or inhibitor of the PD1/PD-L1 axis and the other is an antagonist or an inhibitor of CTLA-4.

In some embodiments, the combination therapy includes the administration of an inhibitor of DGKα and/or DGKζ, the administration of a T cell therapy, and the administration of an antagonist of the PD1/PD-L1 axis. In some embodiments, the combination therapy includes the administration of an inhibitor of DGKα and/or DGKζ, the administration of a T cell therapy, and the administration of an antagonist of CTLA4. In some embodiments, the combination therapy includes the administration of an inhibitor of DGKα and/or DGKζ, the administration of a T cell therapy, the administration of an antagonist of the PD1/PD-L1 axis, and the administration of an antagonist of CTLA4.

In some embodiments, the antagonist of the PD1/PD-L1 axis is nivolumab. In some embodiments, the antagonist of the checkpoint inhibitor is ipilimumab. The antagonist of the checkpoint inhibitor can be any as described in PCT/US202066198, the contents of which are incorporated by reference in their entirety.

### D. Lymphodepleting Treatment

In some aspects, the provided methods can further include administering one or more lymphodepleting therapies, such as prior to or simultaneous with initiation of administration of the T cell therapy. In some embodiments, the lymphodepleting therapy comprises administration of a phosphamide, such as cyclophosphamide. In some embodiments, the lymphodepleting therapy can include administration of fludarabine.

In some aspects, preconditioning subjects with immunodepleting (e.g., lymphodepleting) therapies can improve the effects of adoptive cell therapy (ACT). Preconditioning with lymphodepleting agents, including combinations of cyclosporine and fludarabine, have been effective in improving the efficacy of transferred tumor infiltrating lymphocyte (TIL) cells in cell therapy, including to improve response and/or persistence of the transferred cells. See, e.g., Dudley et al., Science, 298, 850-54(2002); Rosenberg et al., Clin Cancer Res, 17(13): 4550-4557(2011). Likewise, in the context of CAR+ T cells, several studies have incorporated lymphodepleting agents, most commonly cyclophosphamide, fludarabine, bendamustine, or combinations thereof, sometimes accompanied by low-dose irradiation. See Han et al. Journal of Hematology & Oncology, 6:47(2013); Kochenderfer et al., Blood, 119:2709-2720 (2012); Kalos et al., Sci Transl Med, 3(95):95ra73(2011); Clinical Trial Study Record Nos.: NCT02315612; NCT01822652.

Such preconditioning can be carried out with the goal of reducing the risk of one or more of various outcomes that could dampen efficacy of the therapy. These include the phenomenon known as “cytokine sink,” by which T cells, B cells, NK cells compete with TILs for homeostatic and activating cytokines, such as IL-2, IL-7, and/or IL-15; suppression of TILs by regulatory T cells, NK cells, or other cells of the immune system; impact of negative regulators in the tumor microenvironment. Muranski et al., Nat Clin Pract Oncol. December; 3(12): 668-681(2006).

Thus in some embodiments, the provided method further involves administering a lymphodepleting therapy to the subject. In some embodiments, the method involves administering the lymphodepleting therapy to the subject prior to the administration of the dose of cells. In some embodiments, the lymphodepleting therapy contains a chemotherapeutic agent such as fludarabine and/or cyclophosphamide. In some embodiments, the administration of the cells and/or the lymphodepleting therapy is carried out via outpatient delivery.

In some embodiments, the methods include administering a preconditioning agent, such as a lymphodepleting or chemotherapeutic agent, such as cyclophosphamide, fludarabine, or combinations thereof, to a subject prior to the administration of the dose of cells. For example, the subject may be administered a preconditioning agent at least 2 days prior, such as at least 3, 4, 5, 6, or 7 days prior, to the first or subsequent dose. In some embodiments, the subject is administered a preconditioning agent no more than 7 days prior, such as no more than 6, 5, 4, 3, or 2 days prior, to the administration of the dose of cells.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the fludarabine can be administered in a single dose or can be administered in a plurality of doses, such as given daily, every other day or every three days. In some embodiments, the cyclophosphamide is administered once daily for one or two days.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 30 mg/m², or 24 mg/m² and 26 mg/m². In some instances, the subject is administered 25 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (~2 g/m²) of cyclophosphamide and 3 to 5 doses of 25 mg/m² fludarabine prior to the dose of cells.

In some embodiments, the administration of the preconditioning agent prior to infusion of the dose of cells improves an outcome of the treatment. For example, in some aspects, preconditioning improves the efficacy of treatment with the dose or increases the persistence of the recombinant receptor-expressing cells (e.g., CAR-expressing cells, such as CAR-expressing T cells) in the subject. In some embodiments, preconditioning treatment increases disease-free survival, such as the percent of subjects that are alive and exhibit no minimal residual or molecularly detectable disease after a given period of time following the dose of cells. In some embodiments, the time to median disease-free survival is increased.

Once the cells are administered to the subject (e.g., human), the biological activity of the engineered cell populations in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702(2009), and Herman et al. J. Immunological Methods, 285(1): 25-40 (2004). In certain embodiments, the biological activity of the cells also can be measured by assaying expression and/or secretion of certain cytokines, such as CD107a, IFNγ, IL-2, and TNF. In some aspects the biological activity is measured by assessing clinical outcome, such as reduction in tumor burden or load. In some aspects, toxic outcomes, persistence and/or expansion of the cells, and/or presence or absence of a host immune response, are assessed.

In some embodiments, the administration of the preconditioning agent prior to infusion of the dose of cells improves an outcome of the treatment such as by improving the efficacy of treatment with the dose or increases the persistence of the recombinant receptor-expressing cells (e.g., TCR- or CAR-expressing cells) in the subject. Therefore, in some embodiments, the dose of preconditioning agent given in the method which is a combination therapy with the DGK inhibitor and cell therapy is higher than the dose given in the method without the DGK inhibitor.

### II. T CELL THERAPY AND ENGINEERING CELLS

In some embodiments, the T cell therapy for use in accord with the provided combination therapy methods includes administering engineered cells expressing recombinant receptors designed to recognize and/or specifically bind to molecules associated with the disease or condition and result in a response, such as an immune response against such molecules upon binding to such molecules. The receptors may include chimeric receptors, e.g., chimeric antigen receptors (CARs), and other transgenic antigen receptors including transgenic T cell receptors (TCRs).

In some embodiments, the cells contain or are engineered to contain an engineered receptor, e.g., an engineered antigen receptor, such as a chimeric antigen receptor (CAR), or a T cell receptor (TCR). Also provided are populations of such cells, compositions containing such cells and/or enriched for such cells, such as in which cells of a certain type such as T cells or CD8⁺ or CD4⁺ cells are enriched or selected. Among the compositions are pharmaceutical compositions and formulations for administration, such as for adoptive cell therapy. Also provided are therapeutic methods for administering the cells and compositions to subjects, e.g., patients.

Thus, in some embodiments, the cells include one or more nucleic acids introduced via genetic engineering, and thereby express recombinant or genetically engineered products of such nucleic acids. In some embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

### A. Recombinant Receptors

The cells generally express recombinant receptors, such as antigen receptors including functional non-TCR antigen receptors, e.g., chimeric antigen receptors (CARs), and other antigen-binding receptors such as transgenic T cell receptors (TCRs). Also among the receptors are other chimeric receptors.

### 1. Chimeric Antigen Receptors (CARS)

In some embodiments, engineered cells, such as T cells, employed in the provided embodiments express a CAR with specificity for a particular antigen (or marker or ligand), such as an antigen expressed on the surface of a particular cell type. In some embodiments, the antigen is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In particular embodiments, the recombinant receptor, such as chimeric receptor, contains an intracellular signaling region, which includes a cytoplasmic signaling domain or region (also interchangeably called an intracellular signaling domain or region), such as a cytoplasmic (intracellular) region capable of inducing a primary activation signal in a T cell, for example, a cytoplasmic signaling domain or region of a T cell receptor (TCR) component (e.g. a cytoplasmic signaling domain or region of a zeta chain of a CD3-zeta (CD3ζ) chain or a functional variant or signaling portion thereof) and/or that comprises an immunoreceptor tyrosine-based activation motif (ITAM).

In some embodiments, the chimeric receptor further contains an extracellular ligand-binding domain that specifically binds to a ligand (e.g. antigen) antigen. In some embodiments, the chimeric receptor is a CAR that contains an extracellular antigen-recognition domain that specifically binds to an antigen. In some embodiments, the ligand, such as an antigen, is a protein expressed on the surface of cells.

In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule. Generally, a CAR containing an antibody or antigen-binding fragment that exhibits TCR-like specificity directed against peptide-MHC complexes also may be referred to as a TCR-like CAR. In some embodiments, the extracellular antigen binding domain specific for an MHC-peptide complex of a TCR-like CAR is linked to one or more intracellular signaling components, in some aspects via linkers and/or transmembrane domain(s). In some embodiments, such molecules can typically mimic or approximate a signal through a natural antigen receptor, such as a TCR, and, optionally, a signal through such a receptor in combination with a costimulatory receptor.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, WO2016/0046724, WO2016/014789, WO2016/090320, WO2016/094304, WO2017/025038, WO2017/173256, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, 8,479,118, and 9,765,342, and European patent application number EP2537416, and/or those described by Sadelain et al., Cancer Discov., 3(4): 388-398 (2013); Davila et al., PLoS ONE 8(4): e61338 (2013); Turtle et al., Curr. Opin. Immunol., 24(5): 633-39 (2012); Wu et al., Cancer, 18(2): 160-75 (2012), the contents of each of which are incorporated by reference in their entirety. In some aspects, the antigen receptors include a CAR as described in U.S. Pat. No. 7,446,190, and those described in International Patent Application Publication No. WO/2014055668 A1, the contents of each of which are incorporated by reference in their entirety. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., Nature Reviews Clinical Oncology, 10, 267-276 (2013); Wang et al., J. Immunother. 35(9): 689-701 (2012); and Brentjens et al., Sci Transl Med. 5(177) (2013), the contents of each of which are incorporated by reference in their entirety. See also WO2014031687, U.S. Pat. Nos. 8,339,645, 7,446,179, US 2013/0149337, U.S. Pat. Nos. 7,446,190, and 8,389,282, the contents of each of which are incorporated by reference in their entirety. The chimeric receptors, such as CARs, generally include an extracellular antigen binding domain, such as a portion of an antibody molecule, generally a variable heavy (VH) chain region and/or variable light (VL) chain region of the antibody, e.g., an scFv antibody fragment.

In some embodiments, the CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (V_{H}) and variable light (V_{L}) chains of a monoclonal antibody (mAb), or a single domain antibody (sdAb), such as sdFv, nanobody, V_{H}H and V_{NAR}. In some embodiments, an antigen-binding fragment comprises antibody variable regions joined by a flexible linker.

Among the antigen binding domains included in the CARs are antibody fragments. An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; heavy chain variable (V_{H}) regions, single-chain antibody molecules such as scFvs and single-domain antibodies comprising only the V_{H} region; and multispecific antibodies formed from antibody fragments. In particular embodiments, the antibodies are single-chain antibody fragments comprising a heavy chain variable (V_{H}) region and/or a light chain variable (V_{L}) region, such as scFvs.

In certain embodiments, multispecific binding molecules, e.g., multispecific chimeric receptors, such as multispecific CARs, can contain any of the multispecific antibodies, including, e.g. bispecific antibodies, multispecific single-chain antibodies, e.g., diabodies, triabodies, and tetrabodies, tandem di-scFvs, and tandem tri-scFvs.

Single-domain antibodies (sdAbs) are antibody fragments comprising all or a portion of the heavy chain variable region or all or a portion of the light chain variable region of an antibody. In certain embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

In some embodiments, the antibody or antigen-binding fragment thereof is a single-chain antibody fragment, such as a single chain variable fragment (scFv) or a diabody or a single domain antibody (sdAb). In some embodiments, the antibody or antigen-binding fragment is a single domain antibody comprising only the V_{H} region. In some embodiments, the antibody or antigen binding fragment is an scFv comprising a heavy chain variable (V_{H}) region and a light chain variable (V_{L}) region.

In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In certain embodiments, the antigen or includes αvβ6 integrin (avb6 integrin), B cell activating factor receptor (BAFF-R), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD70, CD123, CD133, CD138, CD171, chondroitin sulfate proteoglycan 4 (CSPG4), delta-like ligand 3 (DLL3), epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

Exemplary CAR T cell therapies for use in accordance with the methods provided herein are known in the art. CAR T cell therapies suitable for use in accordance with the methods provided herein include any described in Marofi et al., Stem Cell Res Ther 12:81 (2021); Townsend et al., J Exp Clin Cancer Res 37:163 (2018); Ma et al., Int J Biol Sci 15(12): 2548-2560 (2019); Zhao and Cao, Front Immunol 10:2250 (2019); and Han et al., J Cancer 12(2): 326-334 (2021), the contents of each of which are incorporated by reference herein in their entirety.

Exemplary CAR T cell therapies that target EGFR include those investigated or being investigated in clinical trials NCT03179007, NCT01869166, NCT02331693, NCT03182816, NCT03152435, and NCT03525782. Exemplary CAR T cell therapies that target CD70 include those investigated or being investigated in clinical trials NCT03125577 and NCT028307242. Exemplary CAR T cell therapies that target CD138 include those investigated or being investigated in clinical trials NCT01886976 and NCT03672318. Exemplary CAR T cell therapies that target CD38 include those investigated or being investigated in clinical trials NCT03464916, NCT03473496, NCT03473457, NCT03125577, NCT03222674, and NCT032716322. Exemplary CAR T cell therapies that target CD123 include those investigated or being investigated in clinical trials NCT03473457, NCT03125577, NCT02937103, NCT03114670, NCT02159495, NCT03098355, NCT03222674, NCT03203369, and NCT03190278. Exemplary CAR T cell therapies that target CD133 include those investigated or being investigated in clinical trials NCT03473457, NCT03356782, NCT02541370, and NCT03423992. Exemplary CAR T cell therapies that target GPC3 include those investigated or being investigated in clinical trials NCT02905188, NCT02932956, NCT02715362, NCT03130712, NCT02395250, NCT02876978, NCT03198546, NCT02723942, NCT03084380, NCT03302403, NCT03146234, and NCT02959151. Exemplary CAR T cell therapies that target CD5 include those investigated or being investigated in clinical trial NCT03081910. Exemplary CAR T cell therapies that target ROR1 include those investigated or being investigated in clinical trial NCT02706392. Exemplary CAR T cell therapies that target HerinCAR—PD1 include those investigated or being investigated in clinical trials NCT02873390 and NCT02862028. Exemplary CAR T cell therapies that target HER2 include those investigated or being investigated in clinical trials NCT03500991, NCT03423992, NCT02713984, NCT01935843, NCT03267173, NCT02792114, NCT02442297, NCT00889954, NCT03423992, NCT01109095, NCT02706392, NCT00902044, NCT03389230, NCT02713984, NCT02547961, and NCT01818323. Exemplary CAR T cell therapies that target EGFR806 include those investigated or being investigated in clinical trial NCT03179012. Exemplary CAR T cell therapies that target NY-ESO-1 include those investigated or being investigated in clinical trial NCT03029273. Exemplary CAR T cell therapies that target mesothelin include those investigated or being investigated in clinical trials NCT02930993, NCT03182803, NCT03030001, NCT02706782, NCT01583686, NCT03356795, NCT03054298, NCT03267173, NCT02792114, NCT02959151, NCT02580747, NCT02414269, NCT02465983, NCT03182803, and NCT03323944. Exemplary CAR T cell therapies that target PSCA include those investigated or being investigated in clinical trials NCT03198052, NCT02744287, and NCT03267173. Exemplary CAR T cell therapies that target MG7 include those investigated or being investigated in clinical trial NCT02862704. Exemplary CAR T cell therapies that target MUC1 include those investigated or being investigated in clinical trials NCT03179007, NCT02587689, NCT02617134, NCT03198052, NCT03356795, NCT03267173, NCT03222674, and NCT03356782. Exemplary CAR T cell therapies that target Claudin 18.2 include those investigated or being investigated in clinical trials NCT03874897 and NCT03159819. Exemplary CAR T cell therapies that target EpCAM include those investigated or being investigated in clinical trial NCT02915445, NCT03013712, NCT02729493, NCT02725125, NCT02728882, and NCT02735291. Exemplary CAR T cell therapies that target GD2 include those investigated or being investigated in clinical trials NCT04099797, NCT03423992, NCT03356795, NCT02992210, NCT01953900, NCT02761915, NCT03373097, NCT02765243, NCT03423992, NCT03294954, NCT03356782, and NCT02919046. Exemplary CAR T cell therapies that target VEGFR2 include those investigated or being investigated in clinical trial NCT01218867. Exemplary CAR T cell therapies that target AFP include those investigated or being investigated in clinical trial NCT03349255. Exemplary CAR T cell therapies that target Nectin4/FAP include those investigated or being investigated in clinical trial NCT03932565. Exemplary CAR T cell therapies that target FAP include those investigated or being investigated in clinical trial NCT01722149. Exemplary CAR T cell therapies that target CEA include those investigated or being investigated in clinical trials NCT02850536, NCT02349724, NCT03267173, NCT02959151, and NCT01212887. Exemplary CAR T cell therapies that target Lewis Y include those investigated or being investigated in clinical trial NCT03851146. Exemplary CAR T cell therapies that target Glypican-3 include those investigated or being investigated in clinical trial NCT02932956. Exemplary CAR T cell therapies that target EGFRIII include those investigated or being investigated in clinical trial NCT01454596. Exemplary CAR T cell therapies that target IL-13Rα2 include those investigated or being investigated in clinical trial NCT02208362. Exemplary CAR T cell therapies that target CD171 include those investigated or being investigated in clinical trial NCT02311621. Exemplary CAR T cell therapies that target MUC16 include those investigated or being investigated in clinical trial NCT02311621. Exemplary CAR T cell therapies that target PSMA include those investigated or being investigated in clinical trials NCT03356795, NCT03089203, NCT03185468, and NCT01140373. Exemplary CAR T cell therapies that target AFP include those investigated or being investigated in clinical trial NCT03349255. Exemplary CAR T cell therapies that target AXL include those investigated or being investigated in clinical trial NCT03393936. Exemplary CAR T cell therapies that target CD20 include those investigated or being investigated in clinical trials NCT03893019 and NCT04169932. Exemplary CAR T cell therapies that target CD80/86 include those investigated or being investigated in clinical trial NCT03198052. Exemplary CAR T cell therapies that target CD30 include those investigated or being investigated in clinical trials NCT03383965, NCT04134325, and NCT04008394. Exemplary CAR T cell therapies that target c-MET include those investigated or being investigated in clinical trials NCT03060356 and NCT03638206. Exemplary CAR T cell therapies that target DLL-3 include those investigated or being investigated in clinical trial NCT03392064. Exemplary CAR T cell therapies that target DR5 include those investigated or being investigated in clinical trial NCT03638206. Exemplary CAR T cell therapies that target EpHA2 include those investigated or being investigated in clinical trials NCT02575261 and NCT03423992. Exemplary CAR T cell therapies that target FR-α include those investigated or being investigated in clinical trial NCT00019136. Exemplary CAR T cell therapies that target gp100 include those investigated or being investigated in clinical trial NCT03649529. Exemplary CAR T cell therapies that target IL13Ra2 include those investigated or being investigated in clinical trial NCT02208362. Exemplary CAR T cell therapies that target MAGE-A1/3/4 include those investigated or being investigated in clinical trials NCT03356808 and NCT03535246. Exemplary CAR T cell therapies that target LMP1 include those investigated or being investigated in clinical trial NCT02980315. Exemplary CAR T cell therapies that target EGFRVIII include those investigated or being investigated in clinical trials NCT03283631, NCT02844062, and NCT03170141. Exemplary CAR T cell therapies that target PD-L1 CSR include those investigated or being investigated in clinical trial NCT02937844. Exemplary CAR T cell therapies that target CD19 include those investigated or being investigated in clinical trials NCT02644655, NCT03744676, NCT01087294, NCT03366350, NCT03790891, NCT03497533, NCT04007029, NCT03960840, NCT04049383, NCT04094766, NCT03366324, NCT02546739, NCT03448393, NCT03467256, NCT03488160, NCT04012879, NCT03016377, NCT03468153, NCT03483688, NCT03398967, NCT03229876, NCT03455972, NCT03423706, NCT03497533, and NCT04002401, including FDA-approved products BREYANZI® (lisocabtagene maraleucel), TECARTUS™ (brexucabtagene autoleucel), KYMRIAH™ (tisagenlecleucel), and YESCARTA™ (axicabtagene ciloleucel). Exemplary CAR T cell therapies that target BCMA include those investigated or being investigated in clinical trials NCT03448978, NCT04182581, NCT03271632, NCT03473496, NCT03430011, NCT03455972, NCT02954445, NCT03322735, NCT03338972, NCT03318861, NCT02215967, NCT03093168, NCT03274219, NCT03302403, NCT03492268, NCT03288493, NCT03070327, NCT03196414, NCT03448978, NCT02958410, NCT03287804, NCT03473496, NCT03380039, NCT03430011, NCT03361748, NCT03455972, NCT02546167, NCT03271632, and NCT03548207 (CARVYKTI™ (ciltacabtagene autoleucel)). Exemplary CAR T cell therapies that target BCMA also include FDA-approved products ABECMA® (idecabtagene vicleucel) and CARVYKTI™ (ciltacabtagene autoleucel). Exemplary CAR T cell therapies that target CD33 include those investigated or being investigated in clinical trials NCT03473457, NCT02958397, NCT03126864, and NCT03222674. Exemplary CAR T cell therapies that target GAP include those investigated or being investigated in clinical trial NCT02932956. Exemplary CAR T cell therapies that target Zeushield include those investigated or being investigated in clinical trial NCT03060343. Exemplary CAR T cell therapies that target DLL3 include those investigated or being investigated in clinical trial NCT03392064.

In some embodiments, the CAR is an anti-BCMA CAR that is specific for BCMA, e.g. human BCMA. Chimeric antigen receptors containing anti-BCMA antibodies, including mouse anti-human BCMA antibodies and human anti-human BCMA antibodies, and cells expressing such chimeric receptors have been previously described. See Carpenter et al., Clin Cancer Res., 2013, 19(8): 2048-2060, U.S. Pat. No. 9,765,342, WO 2016/090320, WO2016090327, WO2010104949A2, WO2016/0046724, WO2016/014789, WO2016/094304, WO2017/025038, and WO2017173256, the contents of each of which are incorporated by reference in their entirety.

In some embodiments, the antigen-binding domain of the anti-BCMA CAR is a single-chain antibody fragment, such as a single chain variable fragment (scFv) or a diabody or a single domain antibody (sdAb). In some embodiments, the antigen-binding domain is a single domain antibody (sdAb). In some embodiments, the antigen-binding domain is a single domain antibody comprising only the V_{H} region. In some embodiments, the antigen-binding domain is a single domain antibody comprising only the V_{H} region set forth in SEQ ID NO: 113.

In some embodiments, the anti-BCMA CAR contains an antigen-binding domain, such as an scFv, containing a variable heavy (V_{H}) and/or a variable light (V_{L}) region derived from an antibody described in WO 2016/090320 or WO2016090327, the contents of each of which are incorporated by reference in their entirety. In some embodiments, the antigen-binding domain is an antibody fragment containing a variable heavy chain (V_{H}) and a variable light chain (V_{L}) region. In some aspects, the V_{H} region is or includes an amino acid sequence having at least 90%, 91%, 92%, 93%, 94%, 95%, 96%, 97%, 98% or 99% sequence identity to the V_{H} region amino acid sequence set forth in any of SEQ ID NOs: 30, 32, 34, 36, 38, 40, 42, 77, 79, 81, 83, 85, 87, 89, 91, 93, 95, 97, 99, 101, 103, 105, 107, 109, 111, 113, 181, 183, 185 and 187; and/or the V_{L} region is or includes an amino acid sequence having at least 90%, 91%, 92%, 93%, 94%, 95%, 96%, 97%, 98%, or 99% sequence identity to the V_{L} region amino acid sequence set forth in any of SEQ ID NOs: 31, 33, 35, 37, 39, 41, 43, 78, 80, 82, 84, 86, 88, 90, 92, 94, 96, 98, 100, 102, 104, 106, 108, 110, 112, 182, 184, 186 and 188.

In some embodiments, the antigen-binding domain, such as an scFv, of the anti-BCMA CAR contains a V_{H} set forth in SEQ ID NO: 30 and a V_{L} set forth in SEQ ID NO:31. In some embodiments, the antigen-binding domain, such as an scFv, contains a V_{H} set forth in SEQ ID NO: 32 and a V_{L} set forth in SEQ ID NO:33. In some embodiments, the antigen-binding domain, such as an scFv, contains a V_{H} set forth in SEQ ID NO: 34 and a V_{L} set forth in SEQ ID NO: 35. In some embodiments, the antigen-binding domain, such as an scFv, contains a V_{H} set forth in SEQ ID NO: 36 and a V_{L} set forth in SEQ ID NO:37. In some embodiment the antigen-binding domain, such as an scFv, contains a V_{H} set forth in SEQ ID NO: 38 and a V_{L} set forth in SEQ ID NO: 39. In some embodiments, the antigen-binding domain, such as an scFv, contains a V_{H} set forth in SEQ ID NO: 40 and a V_{L} set forth in SEQ ID NO: 41. In some embodiments, the antigen-binding domain, such as an scFv, contains a V_{H} set forth in SEQ ID NO: 42 and a V_{L} set forth in SEQ ID NO: 43. In some embodiments, the antigen-binding domain, such as an scFv, contains a V_{H} set forth in SEQ ID NO: 77 and a V_{L} set forth in SEQ ID NO: 78. In some embodiments, the antigen-binding domain, such as an scFv, contains a V_{H} set forth in SEQ ID NO: 79 and a V_{L} set forth in SEQ ID NO: 80. In some embodiments, the antigen-binding domain, such as an scFv, contains a V_{H} set forth in SEQ ID NO: 81 and a V_{L} set forth in SEQ ID NO: 82. In some embodiments, the antigen-binding domain, such as an scFv, contains a V_{H} set forth in SEQ ID NO: 83 and a V_{L} set forth in SEQ ID NO: 84. In some embodiments, the antigen-binding domain, such as an scFv, contains a V_{H} set forth in SEQ ID NO: 85 and a V_{L} set forth in SEQ ID NO: 86. In some embodiments, the antigen-binding domain, such as an scFv, contains a V_{H} set forth in SEQ ID NO: 87 and a V_{L} set forth in SEQ ID NO: 88. In some embodiments, the antigen-binding domain, such as an scFv, contains a V_{H} set forth in SEQ ID NO: 89 and a V_{L} set forth in SEQ ID NO: 90. In some embodiments, the antigen-binding domain, such as an scFv, contains a V_{H} set forth in SEQ ID NO: 91 and a V_{L} set forth in SEQ ID NO: 92. In some embodiments, the antigen-binding domain, such as an scFv, contains a V_{H} set forth in SEQ ID NO: 93 and a V_{L} set forth in SEQ ID NO: 94. In some embodiments, the antigen-binding domain, such as an scFv, contains a V_{H} set forth in SEQ ID NO: 95 and a V_{L} set forth in SEQ ID NO: 96. In some embodiments, the antigen-binding domain, such as an scFv, contains a V_{H} set forth in SEQ ID NO: 97 and a V_{L} set forth in SEQ ID NO: 98. In some embodiments, the antigen-binding domain, such as an scFv, contains a V_{H} set forth in SEQ ID NO: 99 and a V_{L} set forth in SEQ ID NO: 100. In some embodiments, the antigen-binding domain, such as an scFv, contains a V_{H} set forth in SEQ ID NO: 101 and a V_{L} set forth in SEQ ID NO: 102. In some embodiments, the antigen-binding domain, such as an scFv, contains a V_{H} set forth in SEQ ID NO: 103 and a V_{L} set forth in SEQ ID NO: 104. In some embodiments, the antigen-binding domain, such as an scFv, contains a V_{H} set forth in SEQ ID NO: 105 and a V_{L} set forth in SEQ ID NO: 106. In some embodiments, the antigen-binding domain, such as an scFv, contains a V_{H} set forth in SEQ ID NO: 107 and a V_{L} set forth in SEQ ID NO: 106. In some embodiments, the antigen-binding domain, such as an scFv, contains a V_{H} set forth in SEQ ID NO: 30 and a V_{L} set forth in SEQ ID NO: 108. In some embodiments, the antigen-binding domain, such as an scFv, contains a V_{H} set forth in SEQ ID NO: 109 and a V_{L} set forth in SEQ ID NO: 110. In some embodiments, the antigen-binding domain, such as an scFv, contains a V_{H} set forth in SEQ ID NO: 111 and a V_{L} set forth in SEQ ID NO: 112. In some embodiments, the antigen-binding domain, such as an scFv, contains a V_{H} set forth in SEQ ID NO: 181 and a V_{L} set forth in SEQ ID NO: 182. In some embodiments, the antigen-binding domain, such as an scFv, contains a V_{H} set forth in SEQ ID NO: 183 and a V_{L} set forth in SEQ ID NO: 184. In some embodiments, the antigen-binding domain, such as an scFv, contains a V_{H} set forth in SEQ ID NO: 185 and a V_{L} set forth in SEQ ID NO: 186. In some embodiments, the antigen-binding domain, such as an scFv, contains a V_{H} set forth in SEQ ID NO: 187 and a V_{L} set forth in SEQ ID NO: 188. In some embodiments, the V_{H} or V_{L} has a sequence of amino acids that exhibits at least 85%, 86%, 87%, 88%, 89%, 90%, 91%, 92%, 93%, 94%, 95%, 96%, 97%, 98%, 99% or more sequence identity to any of the foregoing V_{H} or V_{L} sequences, and retains binding to BCMA. In some embodiments, the V_{H} region is amino-terminal to the V_{L} region. In some embodiments, the V_{H} region is carboxy-terminal to the V_{L} region. In some embodiments, the variable heavy and variable light chains are connected by a linker. In some embodiments, the linker is set forth in SEQ ID NO: 70, 72, 73, 74 or 189.

In some embodiments, the anti-BCMA CAR is any as set forth in SEQ ID NOs: 126-177.

In some embodiments, the anti-BCMA CAR contains the V_{H} and the V_{L} of the anti-BCMA antibody CD115D.3. In some embodiments, the anti-BCMA CAR contains a V_{H} set forth in SEQ ID NO: 30 and a V_{L} set forth in SEQ ID NO: 31. In some embodiments, the anti-BCMA CAR contains an scFv containing the sequence set forth in SEQ ID NO: 249. In some embodiments, the anti-BCMA CAR contains the sequence set forth in SEQ ID NO: 152 (see WO2016/094304). In some embodiments, the anti-BCMA CAR is that of ABECMA (idecabtagene vicleucel (ide-cel)). In some embodiments, the T cell therapy is ABECMA (idecabtagene vicleucel (ide-cel)). In some embodiments,

In some embodiments, the anti-BCMA CAR contains an anti-BCMA single domain antibody. In some embodiments, the anti-BCMA CAR contains two anti-BCMA single domain antibodies. In some embodiments, the anti-BCMA CAR is that of Carvykti (ciltacabtagene autoleucel (cilta-cel)). In some embodiments, the T cell therapy is Carvykti (ciltacabtagene autoleucel (cilta-cel)).

In some embodiments, the anti-BCMA CAR contains a V_{H} set forth in SEQ ID NO: 36 and a V_{L} set forth in SEQ ID NO: 37. In some embodiments, the anti-BCMA CAR contains an scFv containing the sequence set forth in SEQ ID NO: 250. In some embodiments, the anti-BCMA CAR contains the sequence set forth in SEQ ID NO: 160.

In some embodiments, the CAR is an anti-CD19 CAR that is specific for CD19, e.g. human CD19. In some embodiments, the antibody or an antigen-binding fragment (e.g. scFv or V_{H} domain) specifically recognizes an antigen, such as CD19. In some embodiments, the antibody or antigen-binding fragment is derived from, or is a variant of, antibodies or antigen-binding fragment that specifically binds to CD19.

In some embodiments, the antigen is CD19. In some embodiments, the scFv contains a V_{H} and a V_{L} derived from an antibody or an antibody fragment specific to CD19. In some embodiments, the antibody or antibody fragment that binds CD19 is a mouse derived antibody such as FMC63 and SJ25C1. In some embodiments, exemplary antibody or antibody fragment include those described in U.S. Patent Publication No. WO 2014/031687, US 2016/0152723 and WO 2016/033570, the contents of each of which are incorporated by reference in their entirety.

In some embodiments the antigen-binding domain includes a V_{H} and/or V_{L} derived from FMC63, which, in some aspects, can be an scFv. In some embodiments the scFv and/or V_{H} domains is derived from FMC63. FMC63 generally refers to a mouse monoclonal IgG1 antibody raised against Nalm-1 and -16 cells expressing CD19 of human origin (Ling, N. R., et al. (1987). Leucocyte typing III. 302). The FMC63 antibody comprises CDRH1 and H2 set forth in SEQ ID NOS: 44, 45 respectively, and CDRH3 set forth in SEQ ID NOS: 46, 47, or 66 and CDRL1 set forth in SEQ ID NOS: 48 and CDR L2 set forth in SEQ ID NO: 49, 50, or 67 and CDR L3 sequences set forth in SEQ ID NO: 51, 52, or 68. The FMC63 antibody comprises the heavy chain variable region (V_{H}) comprising the amino acid sequence of SEQ ID NO: 53 and the light chain variable region (V_{L}) comprising the amino acid sequence of SEQ ID NO: 54. In some embodiments, the scFv comprises a variable light chain containing the CDRL1 sequence of SEQ ID NO:48, a CDRL2 sequence of SEQ ID NO:49, and a CDRL3 sequence of SEQ ID NO: 51 and/or a variable heavy chain containing a CDRH1 sequence of SEQ ID NO:44, a CDRH2 sequence of SEQ ID NO:45, and a CDRH3 sequence of SEQ ID NO:46. In some embodiments, the scFv comprises a variable heavy chain region of FMC63 set forth in SEQ ID NO: 53 and a variable light chain region of FMC63 set forth in SEQ ID NO:54. In some embodiments, the variable heavy and variable light chains are connected by a linker. In some embodiments, the linker is set forth in SEQ ID NO: 70, 72, 73, 74 or 189. In some embodiments, the scFv comprises, in order, a V_{H}, a linker, and a V_{L}. In some embodiments, the scFv comprises, in order, a V_{L}, a linker, and a V_{H}. In some embodiments, the scFv is encoded by a sequence of nucleotides set forth in SEQ ID NO:69 or a sequence that exhibits at least 85%, 86%, 87%, 88%, 89%, 90%, 91%, 92%, 93%, 94%, 95%, 96%, 97%, 98%, or 99% sequence identity to SEQ ID NO:69. In some embodiments, the scFv comprises the sequence of amino acids set forth in SEQ ID NO:55 or a sequence that exhibits at least 85%, 86%, 87%, 88%, 89%, 90%, 91%, 92%, 93%, 94%, 95%, 96%, 97%, 98%, or 99% sequence identity to SEQ ID NO: 55. In some embodiments, the anti-CD19 CAR is that of BREYANZI® (lisocabtagene maraleucel). In some embodiments, the T cell therapy is BREYANZI® (lisocabtagene maraleucel).

In some embodiments the antigen-binding domain of the anti-CD19 CAR includes a V_{H} and/or V_{L} derived from SJ25C1, which, in some aspects, can be an scFv. SJ25C1 is a mouse monoclonal IgG1 antibody raised against Nalm-1 and -16 cells expressing CD19 of human origin (Ling, N. R., et al. (1987). Leucocyte typing III. 302). The SJ25C1 antibody comprises CDRH1, H2 and H3 set forth in SEQ ID NOS: 59-61, respectively, and CDRL1, L2 and L3 sequences set forth in SEQ ID NOS: 56-58, respectively. The SJ25C1 antibody comprises the heavy chain variable region (V_{H}) comprising the amino acid sequence of SEQ ID NO: 62 and the light chain variable region (V_{L}) comprising the amino acid sequence of SEQ ID NO: 63. In some embodiments, the scFv comprises a variable light chain containing the CDRL1 sequence of SEQ ID NO:56, a CDRL2 sequence of SEQ ID NO: 57, and a CDRL3 sequence of SEQ ID NO: 58 and/or a variable heavy chain containing a CDRH1 sequence of SEQ ID NO:59, a CDRH2 sequence of SEQ ID NO:60, and a CDRH3 sequence of SEQ ID NO:61. In some embodiments, the scFv comprises a variable heavy chain region of SJ25C1 set forth in SEQ ID NO: 62 and a variable light chain region of SJ25C1 set forth in SEQ ID NO:63. In some embodiments, the variable heavy and variable light chain are connected by a linker. In some embodiments, the linker is set forth in SEQ ID NO:64. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO:65 or a sequence that exhibits at least 85%, 86%, 87%, 88%, 89%, 90%, 91%, 92%, 93%, 94%, 95%, 96%, 97%, 98%, or 99% sequence identity to SEQ ID NO:65.

In some embodiments, the anti-CD19 CAR is that of TECARTUS™ (brexucabtagene autoleucel). In some embodiments, the T cell therapy is TECARTUS™ (brexucabtagene autoleucel).

In some embodiments, the anti-CD19 CAR is that of KYMRIAH™ (tisagenlecleucel). In some embodiments, the T cell therapy is KYMRIAH™ (tisagenlecleucel).

In some embodiments, the anti-CD19 CAR is that of YESCARTA™ (axicabtagene ciloleucel). In some embodiments, the T cell therapy is YESCARTA™ (axicabtagene ciloleucel).

In some embodiments, the antigen is CD20. In some embodiments, the scFv contains a V_{H} and a V_{L} derived from an antibody or an antibody fragment specific to CD20. In some embodiments, the antibody or antibody fragment that binds CD20 is an antibody that is or is derived from Rituximab, such as is Rituximab scFv.

In some embodiments, the antigen is CD22. In some embodiments, the scFv contains a V_{H} and a V_{L} derived from an antibody or an antibody fragment specific to CD22. In some embodiments, the antibody or antibody fragment that binds CD22 is an antibody that is or is derived from m⁹⁷¹, such as is m⁹⁷¹ scFv.

In some embodiments, the antigen is ROR1. In some embodiments, the scFv contains a V_{H} and a V_{L} derived from an antibody or an antibody fragment specific to ROR1. In some embodiments, the antibody or antibody fragment that binds ROR1 is or contains a V_{H} and a V_{L} from an antibody or antibody fragment set forth in International Patent Applications, Publication Number WO 2014/031687, WO 2016/115559 and WO 2020/160050, the contents of each of which are incorporated by reference in their entirety.

In some embodiments, the antigen is GPRC5D. In some embodiments, the scFv contains a V_{H} and a V_{L} derived from an antibody or an antibody fragment specific to GPRC5D. In some embodiments, the antibody or antibody fragment that binds GPRC5D is or contains a V_{H} and a V_{L} from an antibody or antibody fragment set forth in International Patent Applications, Publication Number WO 2016/090329, WO 2016/090312 and WO 2020/092854, the contents of each of which are incorporated by reference in their entirety.

In some embodiments, the antigen is FcRL5. In some embodiments, the scFv contains a V_{H} and a V_{L} derived from an antibody or an antibody fragment specific to FcRL5. In some embodiments, the antibody or antibody fragment that binds FcRL5 is or contains a V_{H} and a V_{L} from an antibody or antibody fragment set forth in International Patent Applications, Publication Number WO 2016/090337 and WO 2017/096120, the contents of each of which are incorporated by reference in their entirety.

In some embodiments, the antigen is mesothelin. In some embodiments, the scFv contains a V_{H} and a V_{L} derived from an antibody or an antibody fragment specific to mesothelin. In some embodiments, the antibody or antibody fragment that binds mesothelin is or contains a V_{H} and a V_{L} from an antibody or antibody fragment set forth in US2018/0230429, the contents of which are incorporated by reference in their entirety.

In some embodiments, the antibody is an antigen-binding fragment, such as an scFv, that includes one or more linkers joining two antibody domains or regions, such as a heavy chain variable (V_{H}) region and a light chain variable (V_{L}) region. Accordingly, the antibodies include single-chain antibody fragments, such as scFvs and diabodies, particularly human single-chain antibody fragments, typically comprising linker(s) joining two antibody domains or regions, such V_{H} and V_{L} regions. The linker typically is a peptide linker, e.g., a flexible and/or soluble peptide linker, such as one rich in glycine and serine. Among the linkers are those rich in glycine and serine and/or in some cases threonine. In some embodiments, the linkers further include charged residues such as lysine and/or glutamate, which can improve solubility. In some embodiments, the linkers further include one or more proline.

In some aspects, the linkers rich in glycine and serine (and/or threonine) include at least 80%, 85%, 90%, 91%, 92%, 93%, 94%, 95%, 96%, 97%, 98%, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO:19) or GGGS (3GS; SEQ ID NO:71), such as between 2, 3, 4, and 5 repeats of such a sequence. Exemplary linkers include those having or consisting of an sequence set forth in SEQ ID NO:72 (GGGGGGGGSGGGGS), SEQ ID NO: 189 (ASGGGGSGGRASGGGGS), SEQ ID NO:73 (GSTSGSGKPGSGEGSTKG) or SEQ ID NO: 74 (SRGGGGSGGGGSGGGGSLEMA).

In some embodiments, the recombinant receptor such as the CAR, such as the antibody portion of the recombinant receptor, e.g., CAR, further includes a spacer, which may be or include at least a portion of an immunoglobulin constant region or variant or modified version thereof, such as a hinge region, e.g., an IgG4 hinge region, an IgG1 hinge region, a C_{H}1/C_{L}, and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer.

Exemplary spacers, e.g., hinge regions, include those described in international patent application publication number WO2014031687. In some examples, the spacer is or is about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. In some embodiments, the spacer is a spacer having at least a particular length, such as having a length that is at least 100 amino acids, such as at least 110, 125, 130, 135, 140, 145, 150, 160, 170, 180, 190, 200, 210, 220, 230, 240, or 250 amino acids in length. Exemplary spacers include IgG4 hinge alone, IgG4 hinge linked to C_{H}2 and C_{H}3 domains, or IgG4 hinge linked to the C_{H}3 domain. Exemplary spacers include IgG4 hinge alone, IgG4 hinge linked to C_{H}2 and C_{H}3 domains, or IgG4 hinge linked to the C_{H}3 domain. Exemplary spacers include, but are not limited to, those described in Hudecek et al., Clin. Cancer Res., 19:3153 (2013), Hudecek et al. (2015) Cancer Immunol Res. 3(2): 125-135, international patent application publication number WO2014031687, U.S. Pat. No. 8,822,647 or published app. No. US2014/0271635. In some embodiments, the spacer includes a sequence of an immunoglobulin hinge region, a C_{H}2 and C_{H}3 region. In some embodiments, one of more of the hinge, C_{H}2 and C_{H}3 is derived all or in part from IgG4 or IgG2. In some cases, the hinge, C_{H}2 and C_{H}3 is derived from IgG4. In some aspects, one or more of the hinge, C_{H}2 and C_{H}3 is chimeric and contains sequence derived from IgG4 and IgG2. In some examples, the spacer contains an IgG4/2 chimeric hinge, an IgG2/4 C_{H}2, and an IgG4 C_{H}3 region.

In some embodiments, the spacer, which can be a constant region or portion thereof of an immunoglobulin, is of a human IgG, such as IgG4 or IgG1. In some embodiments, the spacer has the sequence ESKYGPPCPPCP (set forth in SEQ ID NO: 1). In some embodiments, the spacer has the sequence encoded by the nucleotide sequence set forth in SEQ ID NO: 2. In some embodiments, the spacer has the sequence set forth in SEQ ID NO: 3. In some embodiments, the spacer has the sequence set forth in SEQ ID NO: 4. In some embodiments, the encoded spacer is or contains the sequence set forth in SEQ ID NO: 29. In some embodiments, the constant region or portion is of IgD. In some embodiments, the spacer has the sequence set forth in SEQ ID NO: 5. In some embodiments, the spacer has the sequence set forth in SEQ ID NO: 125.

In some embodiments, the spacer can be derived all or in part from IgG4 and/or IgG2 and can contain mutations, such as one or more single amino acid mutations in one or more domains. In some examples, the amino acid modification is a substitution of a proline (P) for a serine(S) in the hinge region of an IgG4. In some embodiments, the amino acid modification is a substitution of a glutamine (Q) for an asparagine (N) to reduce glycosylation heterogeneity, such as an N177Q mutation at position 177, in the C_{H}2 region, of the full-length IgG4 Fc sequence or an N176Q. at position 176, in the CH2 region, of the full-length IgG4 Fc sequence.

Other exemplary spacer regions include hinge regions derived from CD8a, CD28, CTLA4, PD-1, or FcγRIIIa. In some embodiments, the spacer contains a truncated extracellular domain or hinge region of a CD8a, CD28, CTLA4, PD-1, or FcγRIIIa. In some embodiments, the spacer is a truncated CD28 hinge region. In some embodiments, a short oligo- or polypeptide linker, for example, a linker of between 2 and 10 amino acids in length, such as one containing alanines or alanine and arginine, e.g., alanine triplet (AAA) or RAAA (SEQ ID NO: 180), is present and forms a linkage between the scFv and the spacer region of the CAR. In some embodiments, the spacer has the sequence set forth in SEQ ID NO: 114. In some embodiments, the spacer has the sequence set forth in SEQ ID NO: 116. In some embodiments, the spacer has the sequence set forth in any of SEQ ID NOs: 117-119, In some embodiments, the spacer has the sequence set forth in SEQ ID NO: 120. In some embodiments, the spacer has the sequence set forth in SEQ ID NO: 122. In some embodiments, the spacer has the sequence set forth in SEQ ID NO: 124.

In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 1, 3, 4, 5 or 29, 114, 116, 117, 118, 119, 120, 122, 124, or 125.

This antigen recognition domain generally is linked to one or more intracellular signaling components, such as signaling components that mimic stimulation and/or activation through an antigen receptor complex, such as a TCR complex, in the case of a CAR, and/or signal via another cell surface receptor. Thus, in some embodiments, the antigen-binding component (e.g., antibody) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD8a, CD9, CD16, CD22, CD33, CD37, CD64, CD80, CD86, CD 134, CD137 (4-1BB), CD154, CTLA-4, or PD-1. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). Exemplary sequences of transmembrane domains are or comprise the sequences set forth in SEQ ID NOs: 8, 115, 121, 123, 178, or 179.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The receptor, e.g., the CAR, generally includes at least one intracellular signaling component or components. In some embodiments, the receptor includes an intracellular component of a TCR complex, such as a TCR CD3 chain that mediates T-cell stimulation and/or activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25 or CD16. For example, in some aspects, the CAR or other chimeric receptor includes a chimeric molecule between CD3-zeta (CD3-ζ) or Fc receptor γ and CD8, CD4, CD25 or CD16.

In some embodiments, upon ligation of the CAR or other chimeric receptor, the cytoplasmic domain or intracellular signaling domain of the receptor stimulates and/or activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell stimulation and/or activation is in some aspects described as being mediated by two classes of cytoplasmic signaling sequences: those that initiate antigen-dependent primary stimulation and/or activation through the TCR (primary cytoplasmic signaling regions, domains or sequences), and those that act in an antigen-independent manner to provide a secondary or co-stimulatory signal (secondary cytoplasmic signaling regions, domains or sequences). In some aspects, the CAR includes one or both of such signaling components.

In some aspects, the CAR includes a primary cytoplasmic signaling regions, domains or sequence that regulates primary activation of the TCR complex. Primary cytoplasmic signaling regions, domains or sequences that act in a stimulatory manner may contain signaling motifs which are known as immunoreceptor tyrosine-based activation motifs or ITAMs. Examples of ITAM containing primary cytoplasmic signaling sequences include those derived from TCR zeta, FcR gamma, FcR beta, CD3 gamma, CD3 delta, CD3 epsilon, CD8, CD22, CD79a, CD79b and CD66d. In some embodiments, cytoplasmic signaling molecule(s) in the CAR contain(s) a cytoplasmic signaling domain, portion thereof, or sequence derived from CD3 zeta. In some embodiments, the CAR includes a signaling region and/or transmembrane portion of a costimulatory receptor, such as CD28, 4-1BB, OX40 (CD134), CD27, DAP10, DAP12, ICOS and/or other costimulatory receptors. In some aspects, the same CAR includes both the primary cytoplasmic signaling region and costimulatory signaling components.

In some embodiments, one or more different recombinant receptors can contain one or more different intracellular signaling region(s) or domain(s). In some embodiments, the primary cytoplasmic signaling region is included within one CAR, whereas the costimulatory component is provided by another receptor, e.g., another CAR recognizing another antigen. In some embodiments, the CARs include activating or stimulatory CARs, and costimulatory CARs, both expressed on the same cell (see WO2014/055668).

In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and primary cytoplasmic signaling region, in the cytoplasmic portion. Exemplary CARs include intracellular components, such as intracellular signaling region(s) or domain(s), of CD3-zeta, CD28, CD137 (4-1BB), OX40 (CD134), CD27, DAP10, DAP12, NKG2D and/or ICOS. In some embodiments, the chimeric antigen receptor contains an intracellular signaling region or domain of a T cell costimulatory molecule, e.g., from CD28, CD137 (4-1BB), OX40 (CD134), CD27, DAP10, DAP12, NKG2D and/or ICOS, in some cases, between the transmembrane domain and intracellular signaling region or domain. In some aspects, the T cell costimulatory molecule is one or more of CD28, CD137 (4-1BB), OX40 (CD134), CD27, DAP10, DAP12, NKG2D and/or ICOS.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv and the intracellular domain contains an ITAM. In some aspects, the intracellular signaling domain includes a signaling domain of a zeta chain of a CD3-zeta (CD3ζ) chain. In some embodiments, the chimeric antigen receptor includes a transmembrane domain linking the extracellular domain and the intracellular signaling domain. In some aspects, the transmembrane domain contains a transmembrane portion of CD28. In some embodiments, the chimeric antigen receptor contains an intracellular domain of a T cell costimulatory molecule. The extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 4-1BB.

For example, in some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the recombinant receptor, e.g., the CAR, is or includes a transmembrane domain of human CD28 (e.g. Accession No. P10747.1) or CD8a (Accession No. P01732.1) or variant thereof, such as a transmembrane domain that comprises the sequence of amino acids set forth in SEQ ID NO: 8, 115, 178, or 179 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 8, 115, 178, or 179; in some embodiments, the transmembrane-domain containing portion of the recombinant receptor comprises the sequence of amino acids set forth in SEQ ID NO: 9 or a sequence of amino acids having at least at or about 85%, 86%, 87%, 88%, 89%, 90%, 91%, 92%, 93%, 94%, 95%, 96%, 97%, 98%, 99% or more sequence identity thereto.

In some embodiments, the intracellular signaling component(s) of the recombinant receptor, e.g. the CAR, contains an intracellular costimulatory signaling domain of human CD28 or a functional variant or portion thereof, such as a domain with an LL to GG substitution at positions 186-187 of a native CD28 protein. For example, the intracellular signaling domain can comprise the sequence of amino acids set forth in SEQ ID NO: 10 or 11 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 10 or 11. In some embodiments, the intracellular domain comprises an intracellular costimulatory signaling domain of 4-1BB (e.g. Accession No. Q07011.1) or functional variant or portion thereof, such as the sequence of amino acids set forth in SEQ ID NO: 12 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 12.

In some embodiments, the intracellular signaling domain of the recombinant receptor, e.g. the CAR, comprises a human CD3 zeta stimulatory signaling domain or functional variant thereof, such as an 112 AA cytoplasmic domain of isoform 3 of human CD3ζ (Accession No. P20963.2) or a CD3 zeta signaling domain as described in U.S. Pat. No. 7,446,190 or 8,911,993. For example, in some embodiments, the intracellular signaling domain comprises the sequence of amino acids as set forth in SEQ ID NO: 13, 14 or 15 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 13, 14 or 15.

In some aspects, the spacer contains only a hinge region of an IgG, such as only a hinge of IgG4 or IgG1, such as the hinge only spacer set forth in SEQ ID NO: 1 or SEQ ID NO: 125. In other embodiments, the spacer is or contains an Ig hinge, e.g., an IgG4-derived hinge, optionally linked to a CH2 and/or CH3 domains. In some embodiments, the spacer is an Ig hinge, e.g., an IgG4 hinge, linked to CH2 and CH3 domains, such as set forth in SEQ ID NO: 4. In some embodiments, the spacer is an Ig hinge, e.g., an IgG4 hinge, linked to a CH3 domain only, such as set forth in SEQ ID NO: 3. In some embodiments, the spacer is or comprises a glycine-serine rich sequence or other flexible linker such as known flexible linkers. In some embodiments, the spacer is a CD8a hinge, such as set forth in any of SEQ ID NOs: 117-119, an FcγRIIIa hinge, such as set forth in SEQ ID NO: 124, a CTLA4 hinge, such as set forth in SEQ ID NO: 120, or a PD-1 hinge, such as set forth in SEQ ID NO: 122.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition. Non-limiting exemplary CAR sequences are set forth in SEQ ID NOs: 126-177.

In some embodiments, the encoded CAR can sequence can further include a signal sequence or signal peptide that directs or delivers the CAR to the surface of the cell in which the CAR is expressed. In some embodiments, the signal peptide is derived from a transmembrane protein. In some examples the signal peptide is derived from CD8a, CD33, or an IgG. Exemplary signal peptides include the sequences set forth in SEQ ID NOs: 21, 75 and 76 or variant thereof.

In some embodiments, the CAR includes an anti-CD19 antibody such as an antibody fragment, including scFvs, a spacer, such as a spacer containing a portion of an immunoglobulin molecule, such as a hinge region and/or one or more constant regions of a heavy chain molecule, such as any of the Ig-hinge containing spacers or other spacers described herein, a transmembrane domain containing all or a portion of a CD28-derived transmembrane domain, a CD28-derived intracellular signaling domain, and a CD3 zeta signaling domain. In some embodiments, the CAR includes an anti-CD19 antibody or fragment, such as scFv, a spacer such as any of the Ig-hinge containing spacers or other spacers described herein, a CD28-derived transmembrane domain, a 4-1BB-derived intracellular signaling domain, and a CD3 zeta-derived signaling domain. In some embodiments, such CAR constructs further includes a T2A ribosomal skip element and/or a tEGFR sequence, e.g., downstream of the CAR.

In some embodiments, the CAR includes an anti-BCMA antibody or fragment, such as any of the anti-human BCMA antibodies, including sdAbs and scFvs, described herein, a spacer such as any of the Ig-hinge containing spacers or other spacers described herein, a CD28 transmembrane domain, a CD28 intracellular signaling domain, and a CD3 zeta signaling domain. In some embodiments, the CAR includes an anti-BCMA antibody or fragment, such as any of the anti-human BCMA antibodies, including sdAbs and scFvs described herein, a spacer such as any of the Ig-hinge containing spacers or other spacers described herein, a CD28 transmembrane domain, a 4-1BB intracellular signaling domain, and a CD3 zeta signaling domain. In some embodiments, such CAR constructs further includes a T2A ribosomal skip element and/or a tEGFR sequence, e.g., downstream of the CAR. In some embodiments, the CAR includes an anti-BCMA antibody or fragment, such as any of the anti-human BCMA antibodies, including sdAbs and scFvs described herein, a hinge region and transmembrane domain from CD8 alpha, a 4-1BB intracellular signaling domain, and a CD3 zeta signaling domain.

### 2. Chimeric Auto-Antibody Receptor (CAAR)

In some embodiments, the recombinant receptor is a chimeric autoantibody receptor (CAAR). In some embodiments, the CAAR binds, e.g., specifically binds, or recognizes, an autoantibody. In some embodiments, a cell expressing the CAAR, such as a T cell engineered to express a CAAR, can be used to bind to and kill autoantibody-expressing cells, but not normal antibody expressing cells. In some embodiments, CAAR-expressing cells can be used to treat an autoimmune disease associated with expression of self-antigens, such as autoimmune diseases. In some embodiments, CAAR-expressing cells can target B cells that ultimately produce the autoantibodies and display the autoantibodies on their cell surfaces, mark these B cells as disease-specific targets for therapeutic intervention. In some embodiments, CAAR-expressing cells can be used to efficiently targeting and killing the pathogenic B cells in autoimmune diseases by targeting the disease-causing B cells using an antigen-specific chimeric autoantibody receptor. In some embodiments, the recombinant receptor is a CAAR, such as any described in U.S. Patent Application Pub. No. US 2017/0051035.

In some embodiments, the CAAR comprises an autoantibody binding domain, a transmembrane domain, and one or more intracellular signaling region or domain (also interchangeably called a cytoplasmic signaling domain or region). In some embodiments, the intracellular signaling region comprises an intracellular signaling domain. In some embodiments, the intracellular signaling domain is or comprises a primary signaling region, a signaling domain that is capable of stimulating and/or inducing a primary activation signal in a T cell, a signaling domain of a T cell receptor (TCR) component (e.g. an intracellular signaling domain or region of a zeta chain of a CD3-zeta (CD3ζ) chain or a functional variant or signaling portion thereof), and/or a signaling domain comprising an immunoreceptor tyrosine-based activation motif (ITAM).

In some embodiments, the autoantibody binding domain comprises an autoantigen or a fragment thereof. The choice of autoantigen can depend upon the type of autoantibody being targeted. For example, the autoantigen may be chosen because it recognizes an autoantibody on a target cell, such as a B cell, associated with a particular disease state, e.g. an autoimmune disease, such as an autoantibody-mediated autoimmune disease. In some embodiments, the autoimmune disease includes pemphigus vulgaris (PV). Exemplary autoantigens include desmoglein 1 (Dsg1) and Dsg3.

### 3. TCRS

In some embodiments, engineered cells, such as T cells, are provided that express a T cell receptor (TCR) or antigen-binding portion thereof that recognizes an peptide epitope or T cell epitope of a target polypeptide, such as an antigen of a tumor, viral or autoimmune protein. In some aspects, the TCR is or includes a recombinant TCR.

In some embodiments, a “T cell receptor” or “TCR” is a molecule that contains a variable α and β chains (also known as TCRα and TCRβ, respectively) or a variable γ and δ chains (also known as TCRα and TCRβ, respectively), or antigen-binding portions thereof, and which is capable of specifically binding to a peptide bound to an MHC molecule. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions involved in recognition of the peptide, MHC and/or MHC-peptide complex.

In some embodiments, the variable domains of the TCR contain hypervariable loops, or complementarity determining regions (CDRs), which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the β-chain can contain a further hypervariable region (CDR4 or HVR4), which generally is involved in superantigen binding and not antigen recognition (Kotb (1995) Clinical Microbiology Reviews, 8:411-426).

In some embodiments, a TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). In some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR chain contains one or more constant domain. For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) and a constant domain (e.g., α-chain constant domain or Cα, typically positions 117 to 259 of the chain based on Kabat numbering or β chain constant domain or Cβ, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains.

In some embodiments, the TCR chains contain a transmembrane domain. In some embodiments, the transmembrane domain is positively charged. In some cases, the TCR chain contains a cytoplasmic tail. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3γ, CD3δ, CD3ε and CD3ζ chains) contain one or more immunoreceptor tyrosine-based activation motif or ITAM that are involved in the signaling capacity of the TCR complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, the TCR can be generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, the recombinant receptors include recombinant TCRs and/or TCRs cloned from naturally occurring T cells. In some embodiments, a high-affinity T cell clone for a target antigen (e.g., a cancer antigen) is identified, isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354.

In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, the TCR is a thymically selected TCR. In some embodiments, the TCR is a neoepitope-restricted TCR. In some embodiments, the T-cells can be a cultured T-cell hybridoma or clone. In some embodiments, the TCR or antigen-binding portion thereof can be synthetically generated from knowledge of the sequence of the TCR.

In some embodiments, the TCR is generated from a TCR identified or selected from screening a library of candidate TCRs against a target polypeptide antigen, or target T cell epitope thereof. TCR libraries can be generated by amplification of the repertoire of Vα and Vβ from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4⁺ or CD8⁺ cells. In some embodiments, the TCRs can be amplified from a T cell source of a normal of healthy subject, i.e. normal TCR libraries. In some embodiments, the TCRs can be amplified from a T cell source of a diseased subject, i.e. diseased TCR libraries. In some embodiments, degenerate primers are used to amplify the gene repertoire of Vα and Vβ, such as by RT-PCR in samples, such as T cells, obtained from humans. In some embodiments, scTv libraries can be assembled from naïve Vα and Vβ libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule. In some aspects, the TCRs are subjected to directed evolution, such as by mutagenesis, e.g., of the α or β chain. In some aspects, particular residues within CDRs of the TCR are altered. In some embodiments, selected TCRs can be modified by affinity maturation. In some embodiments, antigen-specific T cells may be selected, such as by screening to assess CTL activity against the peptide. In some aspects, TCRs, e.g. present on the antigen-specific T cells, may be selected, such as by binding activity, e.g., particular affinity or avidity for the antigen.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al., (2003) Nat Immunol, 4, 55-62; Holler et al., (2000) Proc Natl Acad Sci USA, 97, 5387-92), phage display (Li et al., (2005) Nat Biotechnol, 23, 349-54), or T cell display (Chervin et al., (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

Exemplary TCR T cell therapies for use in accordance with the methods provided herein are known in the art. TCR T cell therapies suitable for use in accordance with the methods provided herein include any described in Zhao and Cao, Front Immunol 10:2250 (2019); Ping et al., Protein Cell 9(3): 254-266 (2018); and Zhang and Wang, Technol Cancer Res Treat 18:1533033819831068 (2019), the contents of each of which are incorporated by reference herein in their entirety.

Exemplary TCR T cell therapies that target PRAME include those investigated or being investigated in clinical trials NCT03503968 and NCT02743611. Exemplary TCR T cell therapies that target MAGE-A3/A6 include those investigated or being investigated in clinical trial NCT03139370. Exemplary TCR T cell therapies that target CEA include those investigated or being investigated in clinical trial NCT00923806. Exemplary TCR T cell therapies that target MAGE-A3/12 include those investigated or being investigated in clinical trial NCT01273181. Exemplary TCR T cell therapies that target MAGE-A10 include those investigated or being investigated in clinical trials NCT02592577, NCT03391791, and NCT02989064. Exemplary TCR T cell therapies that target NY-ESO-1 include those investigated or being investigated in clinical trials NCT01343043, NCT03029273, NCT03462316, NCT01892293, NCT01352286, NCT01567891, NCT01350401, NCT02588612, NCT03691376, and NCT03168438. Exemplary TCR T cell therapies that target AFP include those investigated or being investigated in clinical trial NCT03132792. Exemplary TCR T cell therapies that target HA-1 include those investigated or being investigated in clinical trial NCT03326921. Exemplary TCR T cell therapies that target WT1 include those investigated or being investigated in clinical trials NCT02550535 and NCT02770820. Exemplary TCR T cell therapies that target Gp100 include those investigated or being investigated in clinical trials NCT00923195 and NCT02889861. Exemplary TCR T cell therapies that target CMV include those investigated or being investigated in clinical trial NCT02988258. Exemplary TCR T cell therapies that target MART-1 include those investigated or being investigated in clinical trial NCT00091104. Exemplary TCR T cell therapies that target HBV include those investigated or being investigated in clinical trial NCT02719782. Exemplary TCR T cell therapies that target P53 include those investigated or being investigated in clinical trial NCT00393029. Exemplary TCR T cell therapies that target HPV-16 E6 include those investigated or being investigated in clinical trials NCT03578406 and NCT02280811. Exemplary TCR T cell therapies that target HPV-16 E7 include those investigated or being investigated in clinical trial NCT02858310. Exemplary TCR T cell therapies that target SL9 include those investigated or being investigated in clinical trial NCT00991224. Exemplary TCR T cell therapies that target TGFβII include those investigated or being investigated in clinical trial NCT03431311. Exemplary TCR T cell therapies that target MCPy V include those investigated or being investigated in clinical trial NCT03747484. Exemplary TCR T cell therapies that target TRAIL include those investigated or being investigated in clinical trial NCT00923390. Exemplary TCR T cell therapies that target EBV include those investigated or being investigated in clinical trial NCT03648697. Exemplary TCR T cell therapies that target KRAS include those investigated or being investigated in clinical trials NCT03190941 and NCT03745326.

In some embodiments, peptides of a target polypeptide for use in producing or generating a TCR of interest are known or can be readily identified as a matter of routine. In some embodiments, peptides suitable for use in generating TCRs or antigen-binding portions can be determined based on the presence of an HLA-restricted motif in a target polypeptide of interest, such as a target polypeptide described below. In some embodiments, peptides are identified using computer prediction models as a matter of routine. In some embodiments, for predicting MHC class I binding sites, such models include, but are not limited to, ProPred1 (Singh and Raghava (2001) Bioinformatics 17(12): 1236-1237, and SYFPEITHI (see Schuler et al., (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule.

HLA-A0201-binding motifs and the cleavage sites for proteasomes and immune-proteasomes using computer prediction models are known. For predicting MHC class I binding sites, such models include, but are not limited to, ProPred1 (described in more detail in Singh and Raghava, ProPred: prediction of HLA-DR binding sites. BIOINFORMATICS 17(12): 1236-1237 2001), and SYFPEITHI (see Schuler et al., SYFPEITHI, Database for Searching and T-Cell Epitope Prediction. in Immunoinformatics Methods in Molecular Biology, vol 409(1): 75-93 2007)

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, for purposes of the provided methods, the TCR is in cell-bound form expressed on the surface of a cell.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). In some embodiments, a dTCR or scTCR have the structures as described in WO 03/020763, WO 04/033685, WO2011/044186.

In some embodiments, the TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the TCRs, including a dTCR or scTCR, can be linked to signaling domains that yield an active TCR on the surface of a T cell. In some embodiments, the TCR is expressed on the surface of cells.

In some embodiments a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native inter-chain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR α chain containing a variable α domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR. Typically, a scTCR can be generated using suitable known methods, See e.g., Soo Hoo, W. F. et al., PNAS (USA) 89, 4759 (1992); Wülfing, C. and Plückthun, A., J. Mol. Biol. 242, 655 (1994); Kurucz, I. et al., PNAS (USA) 90 3830 (1993); International published PCT Nos. WO 96/13593, WO 96/18105, WO99/60120, WO99/18129, WO 03/020763, WO2011/044186; and Schlueter, C. J. et al., J. Mol. Biol. 256, 859 (1996). In some embodiments, a scTCR contains an introduced non-native disulfide interchain bond to facilitate the association of the TCR chains (see e.g. International published PCT No. WO 03/020763). In some embodiments, a scTCR is a non-disulfide linked truncated TCR in which heterologous leucine zippers fused to the C-termini thereof facilitate chain association (see e.g. International published PCT No. WO99/60120). In some embodiments, a scTCR contain a TCRα variable domain covalently linked to a TCRβ variable domain via a peptide linker (see e.g., International published PCT No. WO99/18129).

In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an α chain variable region sequence fused to the N terminus of an α chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a β chain extracellular constant domain sequence, and a second segment constituted by an α chain variable region sequence fused to the N terminus of a sequence α chain extracellular constant and transmembrane sequence, and, optionally, a linker sequence linking the C terminus of the first segment to the N terminus of the second segment.

In some embodiments, the linker of a scTCRs that links the first and second TCR segments can be any linker capable of forming a single polypeptide strand, while retaining TCR binding specificity. In some embodiments, the linker sequence may, for example, have the formula —P-AA-P— wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG) 5-P- wherein P is proline, G is glycine and S is serine (SEQ ID NO: 16). In some embodiments, the linker has the sequence GSADDAKKDAAKKDGKS (SEQ ID NO: 17)

In some embodiments, the scTCR contains a covalent disulfide bond linking a residue of the immunoglobulin region of the constant domain of the α chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments of a dTCR or scTCR containing introduced interchain disulfide bonds, the native disulfide bonds are not present. In some embodiments, the one or more of the native cysteines forming a native interchain disulfide bonds are substituted to another residue, such as to a serine or alanine. In some embodiments, an introduced disulfide bond can be formed by mutating non-cysteine residues on the first and second segments to cysteine. Exemplary non-native disulfide bonds of a TCR are described in published International PCT No. WO2006/000830.

In some embodiments, the TCR or antigen-binding fragment thereof exhibits an affinity with an equilibrium binding constant for a target antigen of between or between about 10⁻⁵ and 10⁻¹² M and all individual values and ranges therein. In some embodiments, the target antigen is an MHC-peptide complex or ligand.

In some aspects, the TCR or antigen-binding fragment thereof binds to a peptide epitope derived from HPV16 E6 protein, HPV16 E7 protein and/or to a peptide epitope expressed on a cell infected with HPV. In some embodiments, the TCR or antigen-binding fragment thereof binds or recognizes a peptide epitope of HPV 16 E6 or E7, in the context of an MHC molecule. HPV is a causative organism in most cases of cervical cancer and has been implicated in anal, vaginal, vulvar, penile, and oropharyngeal cancers, and other cancers. Generally, the HPV genome contains an early region containing six open reading frames (E1, E2, E4, E5, E6 and E7), which encode proteins involved in cell transformation and replication, and a late region containing two open reading frames (L1 and L2), which encode proteins of the viral capsid. In general, E6 and E7 are oncogenes that can affect cell cycle regulation and contribute to the formation of cancers. For instance, the E6 gene product can cause p53 degradation and the E7 gene product can cause retinoblastoma (Rb) inactivation.

In some aspects, the TCR or antigen-binding fragment thereof recognizes or binds HPV 16 E6 or E7 epitopes in the context of an MHC molecule, such as an MHC Class I molecule. In some aspects, the MHC Class I molecule is a human leukocyte antigen (HLA)-A2 molecule, including any one or more subtypes thereof, e.g. HLA-A*0201, *0202, *0203, *0206, or *0207. In some cases, there can be differences in the frequency of subtypes between different populations. For example, in some embodiments, more than 95% of the HLA-A2 positive Caucasian population is HLA-A*0201, whereas in the Chinese population the frequency has been reported to be approximately 23% HLA-A*0201, 45% HLA-A*0207, 8% HLA-A*0206 and 23% HLA-A*0203. In some embodiments, the MHC molecule is HLA-A*0201.

In some embodiments, the TCR or antigen-binding fragment thereof recognizes or binds to an epitope or region of HPV16 E6 or HPV 16 E7, such as a peptide epitope containing an amino acid sequence set forth in any of SEQ ID NOs: 192-199, and as shown below in Table 1. In some embodiments, the TCR or antigen-binding fragment thereof has antigenic specificity for HPV 16 E7 protein or a portion of HPV 16 E7 protein. In some embodiments, the TCR or antigen-binding fragment thereof recognizes or binds to a peptide epitope derived from HPV 16 E7 that is or comprises E7(11-19) YMLDLQPET (SEQ ID NO: 196).

TABLE 1
HPV-16 Epitopes

| Epitope Description | Epitope Name | SEQ ID NO. |
|---|---|---|
| KLPQLCTEL | E6(18-26) | 192 |
| TIHDIILECV | E6(29-38) | 193 |
| FAFRDLCIV | E6(52-60) | 194 |
| TLGIVCPI | E7(86-93) | 195 |
| YMLDLQPET | E7(11-19) | 196 |
| GTLGIVCPI | E7(85-93) | 197 |
| LLMGTLGIV | E7(82-90) | 198 |
| TLHEYMLDL | E7(7-15) | 199 |

In some embodiments, the TCR or antigen-binding fragment thereof that recognizes or binds to an HPV 16 epitope includes any as described in US20190062398A1; US20190225692A1; US20190321401A1; and WO2019070541A1, the contents of each of which are incorporated by reference in their entirety. In some embodiments, the TCR or antigen-binding fragment thereof comprises TCR sequences (e.g., the combination of alpha and beta chains) found in US20190062398A1; US20190225692A1; US20190321401A1; and WO2019070541A1 that recognize or bind to HPV 16 E7 protein or a portion of HPV 16 E7 protein. In some embodiments, the TCR or antigen-binding fragment thereof comprises TCR sequences (e.g., the combination of alpha and beta chains) found in US20190062398A1; US20190225692A1; US20190321401A1; and WO2019070541A1 that recognize or bind to HPV 16 E7(11-19).

In some embodiments, a TCR or functional variant thereof comprises an alpha chain containing a variable alpha (Vα) chain with a CDR1, CDR2 and CDR3 as contained in the Vα chain set forth in SEQ ID NO:202, and a beta chain containing a variable beta (Vβ) chain with a CDR1, CDR2 and CDR3 as contained in the Vβ chain set forth in SEQ ID NO:208. In some embodiments, a TCR or functional variant thereof comprises an alpha chain containing a Vα chain with a CDR1, a CDR2 and a CDR3 set forth in SEQ ID NOS: 203, 204 and 205, respectively, and a Vβ chain with a CDR1, CDR2 and CDR3 set forth in SEQ ID NOS: 209, 210 and 211, respectively. In some embodiments, a TCR or functional variant thereof comprises an alpha chain containing a Vα chain set forth in SEQ ID NO:202, and a beta chain containing a Vβ chain set forth in SEQ ID NO:208. In some embodiments, a TCR or functional variant thereof comprises an alpha chain set forth in SEQ ID NO:201, and a beta chain set forth in SEQ ID NO: 207. In some embodiments, a TCR or functional variant thereof contains an alpha chain encoded by the nucleotide sequence set forth in SEQ ID NO:200, and a beta chain encoded by the nucleotide sequence set forth in SEQ ID NO:206. In some embodiments, the TCR or functional variant thereof is encoded by the nucleotide sequence set forth in SEQ ID NO:212.

In some embodiments, a TCR or functional variant thereof comprises an alpha chain containing a variable alpha (Vα) chain with a CDR1, CDR2 and CDR3 as contained in the Vα chain set forth in SEQ ID NO:215, and a beta chain containing a variable beta (Vβ) chain with a CDR1, CDR2 and CDR3 as contained in the Vβ chain set forth in SEQ ID NO:218. In some embodiments, a TCR or functional variant thereof comprises an alpha chain containing a Vα chain with a CDR1, a CDR2 and a CDR3 set forth in SEQ ID NOS: 219, 220 and 221, respectively, and a Vβ chain with a CDR1, CDR2 and CDR3 set forth in SEQ ID NOS: 222, 223 and 224, respectively. In some embodiments, a TCR or functional variant thereof comprises an alpha chain containing a Vα chain set forth in SEQ ID NO:215, and a beta chain containing a Vβ chain set forth in SEQ ID NO:218. In some embodiments, a TCR or functional variant thereof comprises an alpha chain set forth in SEQ ID NO:214, and a beta chain set forth in SEQ ID NO: 217. In some embodiments, a TCR or functional variant thereof contains an alpha chain encoded by the nucleotide sequence set forth in SEQ ID NO:213, and a beta chain encoded by the nucleotide sequence set forth in SEQ ID NO:216.

In some embodiments, a TCR or functional variant thereof comprises an alpha chain containing a variable alpha (Vα) chain with a CDR1, CDR2 and CDR3 as contained in the Vα chain set forth in SEQ ID NO:227, and a beta chain containing a variable beta (Vβ) chain with a CDR1, CDR2 and CDR3 as contained in the Vβ chain set forth in SEQ ID NO:230. In some embodiments, a TCR or functional variant thereof comprises an alpha chain containing a Vα chain with a CDR1, a CDR2 and a CDR3 set forth in SEQ ID NOS: 231, 232 and 233, respectively, and a Vβ chain with a CDR1, CDR2 and CDR3 set forth in SEQ ID NOS: 234, 235 and 236, respectively. In some embodiments, a TCR or functional variant thereof comprises an alpha chain containing a Vα chain set forth in SEQ ID NO:227, and a beta chain containing a Vβ chain set forth in SEQ ID NO:230. In some embodiments, a TCR or functional variant thereof comprises an alpha chain set forth in SEQ ID NO:226, and a beta chain set forth in SEQ ID NO: 229. In some embodiments, a TCR or functional variant thereof contains an alpha chain encoded by the nucleotide sequence set forth in SEQ ID NO:225, and a beta chain encoded by the nucleotide sequence set forth in SEQ ID NO:228.

In some embodiments, a TCR or functional variant thereof comprises an alpha chain containing a variable alpha (Vα) chain with a CDR1, CDR2 and CDR3 as contained in the Vα chain set forth in SEQ ID NO:239, and a beta chain containing a variable beta (Vβ) chain with a CDR1, CDR2 and CDR3 as contained in the Vβ chain set forth in SEQ ID NO:242. In some embodiments, a TCR or functional variant thereof comprises an alpha chain containing a Vα chain with a CDR1, a CDR2 and a CDR3 set forth in SEQ ID NOS: 243, 244 and 245, respectively, and a Vβ chain with a CDR1, CDR2 and CDR3 set forth in SEQ ID NOS: 246, 247 and 248, respectively. In some embodiments, a TCR or functional variant thereof comprises an alpha chain containing a Vα chain set forth in SEQ ID NO:239, and a beta chain containing a Vβ chain set forth in SEQ ID NO:242. In some embodiments, a TCR or functional variant thereof comprises an alpha chain set forth in SEQ ID NO:238, and a beta chain set forth in SEQ ID NO: 241. In some embodiments, a TCR or functional variant thereof contains an alpha chain encoded by the nucleotide sequence set forth in SEQ ID NO:237, and a beta chain encoded by the nucleotide sequence set forth in SEQ ID NO:240.

In some embodiments, nucleic acid or nucleic acids encoding a TCR, such as α and β chains, can be amplified by PCR, cloning or other suitable means and cloned into a suitable expression vector or vectors. The expression vector can be any suitable recombinant expression vector, and can be used to transform or transfect any suitable host. Suitable vectors include those designed for propagation and expansion or for expression or both, such as plasmids and viruses.

In some embodiments, the vector can a vector of the pUC series (Fermentas Life Sciences), the pBluescript series (Stratagene, La Jolla, Calif.), the pET series (Novagen, Madison, Wis.), the pGEX series (Pharmacia Biotech, Uppsala, Sweden), or the pEX series (Clontech, Palo Alto, Calif.). In some cases, bacteriophage vectors, such as λG10, λGT11, λZapII (Stratagene), λEMBL4, and λNM1149, also can be used. In some embodiments, plant expression vectors can be used and include pBI01, pBI101.2, pBI101.3, pBI121 and pBIN19 (Clontech). In some embodiments, animal expression vectors include pEUK-C1, pMAM and pMAMneo (Clontech). In some embodiments, a viral vector is used, such as a retroviral vector.

In some embodiments, the recombinant expression vectors can be prepared using standard recombinant DNA techniques. In some embodiments, vectors can contain regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In some embodiments, the vector can contain a nonnative promoter operably linked to the nucleotide sequence encoding the TCR or antigen-binding portion (or other MHC-peptide binding molecule). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, to generate a vector encoding a TCR, the α and β chains are PCR amplified from total cDNA isolated from a T cell clone expressing the TCR of interest and cloned into an expression vector. In some embodiments, the α and β chains are cloned into the same vector. In some embodiments, the α and β chains are cloned into different vectors. In some embodiments, the generated α and β chains are incorporated into a retroviral, e.g. lentiviral, vector.

### 4. Multi-Targeting

In some embodiments, the cells and methods include multi-targeting strategies, such as expression of two or more genetically engineered receptors on the cell, each recognizing the same of a different antigen and typically each including a different intracellular signaling component. Such multi-targeting strategies are described, for example, in PCT Pub. No. WO 2014055668 A1 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

For example, in some embodiments, the cells include a receptor expressing a first genetically engineered antigen receptor (e.g., CAR or TCR) which is capable of inducing an activating signal to the cell, generally upon specific binding to the antigen recognized by the first receptor, e.g., the first antigen. In some embodiments, the cell further includes a second genetically engineered antigen receptor (e.g., CAR or TCR), e.g., a chimeric costimulatory receptor, which is capable of inducing a costimulatory signal to the immune cell, generally upon specific binding to a second antigen recognized by the second receptor. In some embodiments, the first antigen and second antigen are the same. In some embodiments, the first antigen and second antigen are different.

In some embodiments, the first and/or second genetically engineered antigen receptor (e.g. CAR or TCR) is capable of inducing an activating signal to the cell. In some embodiments, the receptor includes an intracellular signaling component containing ITAM or ITAM-like motifs. In some embodiments, the activation induced by the first receptor involves a signal transduction or change in protein expression in the cell resulting in initiation of an immune response, such as ITAM phosphorylation and/or initiation of ITAM-mediated signal transduction cascade, formation of an immunological synapse and/or clustering of molecules near the bound receptor (e.g. CD4 or CD8, etc.), activation of one or more transcription factors, such as NF-κB and/or AP-1, and/or induction of gene expression of factors such as cytokines, proliferation, and/or survival.

In some embodiments, the first and/or second receptor includes intracellular signaling domains of costimulatory receptors such as CD28, CD137 (4-1 BB), OX40, and/or ICOS. In some embodiments, the first and second receptors include an intracellular signaling domain of a costimulatory receptor that are different. In one embodiment, the first receptor contains a CD28 costimulatory signaling region and the second receptor contain a 4-1BB co-stimulatory signaling region or vice versa.

In some embodiments, the first and/or second receptor includes both an intracellular signaling domain containing ITAM or ITAM-like motifs and an intracellular signaling domain of a costimulatory receptor.

In some embodiments, the first receptor contains an intracellular signaling domain containing ITAM or ITAM-like motifs and the second receptor contains an intracellular signaling domain of a costimulatory receptor. The costimulatory signal in combination with the activating signal induced in the same cell is one that results in an immune response, such as a robust and sustained immune response, such as increased gene expression, secretion of cytokines and other factors, and T cell mediated effector functions such as cell killing.

In some embodiments, neither ligation of the first receptor alone nor ligation of the second receptor alone induces a robust immune response. In some aspects, if only one receptor is ligated, the cell becomes tolerized or unresponsive to antigen, or inhibited, and/or is not induced to proliferate or secrete factors or carry out effector functions. In some such embodiments, however, when the plurality of receptors are ligated, such as upon encounter of a cell expressing the first and second antigens, a desired response is achieved, such as full immune activation or stimulation, e.g., as indicated by secretion of one or more cytokine, proliferation, persistence, and/or carrying out an immune effector function such as cytotoxic killing of a target cell.

In some embodiments, the cells expressing the recombinant receptor further include inhibitory CARs (iCARs, see Fedorov et al., Sci. Transl. Medicine, 5 (215) (2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In some embodiments, the two receptors induce, respectively, an activating and an inhibitory signal to the cell, such that binding by one of the receptor to its antigen activates the cell or induces a response, but binding by the second inhibitory receptor to its antigen induces a signal that suppresses or dampens that response. Examples are combinations of activating CARs and inhibitory CARs or iCARs. Such a strategy may be used, for example, in which the activating CAR binds an antigen expressed in a disease or condition but which is also expressed on normal cells, and the inhibitory receptor binds to a separate antigen which is expressed on the normal cells but not cells of the disease or condition.

In some aspects, the chimeric receptor is or includes an inhibitory CAR (e.g. iCAR) and includes intracellular components that dampen or suppress an immune response, such as an ITAM- and/or co stimulatory-promoted response in the cell. Exemplary of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell, for example, that induced by an activating and/or costimulatory CAR.

In some embodiments, the multi-targeting strategy is employed in a case where an antigen associated with a particular disease or condition is expressed on a non-diseased cell and/or is expressed on the engineered cell itself, either transiently (e.g., upon stimulation in association with genetic engineering) or permanently. In such cases, by requiring ligation of two separate and individually specific antigen receptors, specificity, selectivity, and/or efficacy may be improved.

In some embodiments, the plurality of antigens, e.g., the first and second antigens, are expressed on the cell, tissue, or disease or condition being targeted, such as on the cancer cell. In some aspects, the cell, tissue, disease or condition is multiple myeloma or a multiple myeloma cell. In some embodiments, one or more of the plurality of antigens generally also is expressed on a cell which it is not desired to target with the cell therapy, such as a normal or non-diseased cell or tissue, and/or the engineered cells themselves. In such embodiments, by requiring ligation of multiple receptors to achieve a response of the cell, specificity and/or efficacy is achieved.

### B. Cells and Preparation of Cells for Genetic Engineering

Among the cells expressing the receptors and administered by the provided methods are engineered cells. The genetic engineering generally involves introduction of a nucleic acid encoding the recombinant or engineered component into a composition containing the cells, such as by retroviral transduction, transfection, or transformation.

In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4⁺ and/or of CD8⁺ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, genes and/or gene products (and/or expression thereof) in the provided cells, and/or compositions containing such cells, are reduced, deleted, eliminated, knocked-out or disrupted. Such genes and/or gene products in some aspects include one or more of the gene encoding (or product thereof) TCR alpha (TRAC) and/or TCR beta (TRBC), e.g., to reduce or prevent expression of the endogenous TCR in the cell, e.g. T cell, and/or a chain thereof. In some embodiments, the cell, e.g., T cell, expresses an engineered TCR (e.g., any as described in Section II-A-3). In some embodiments, reducing or preventing endogenous TCR expression can lead to a reduced risk or chance of mispairing between chains of the engineered TCR and the endogenous TCR, thereby creating a new TCR that could potentially result in a higher risk of undesired or unintended antigen recognition and/or side effects, and/or could reduce expression levels of the desired exogenous TCR. In some aspects, reducing or preventing endogenous TCR expression can increase expression of the engineered TCR in the cells as compared to cells in which expression of the TCR is not reduced or prevented, such as increased by 1.5-fold, 2-fold, 3-fold, 4-fold, 5-fold or more. For example, in some cases, suboptimal expression of an engineered or recombinant TCR can occur due to competition with an endogenous TCR and/or with TCRs having mispaired chains, for the invariant CD3 signaling molecules that are involved in permitting expression of the complex on the cell surface. In some embodiments, reduction, deletion, elimination, knockout or disruption is carried out by gene editing, such as using a zinc finger nuclease (ZFN), TALEN or a CRISPR/Cas system with an engineered single guide RNA (gRNA) that cleaves a TCR gene. In some embodiments, reducing expression of an endogenous TCR is carried out using an inhibitory nucleic acid molecule against a target nucleic acids encoding specific TCRs (e.g., TCR-α and TCR-β). In some embodiments, the inhibitory nucleic acid is or contains or encodes a small interfering RNA (siRNA), a microRNA-adapted shRNA, a short hairpin RNA (shRNA), a hairpin siRNA, a microRNA (miRNA-precursor) or a microRNA (miRNA). Exemplary methods for reducing or preventing endogenous TCR expression are known in the art, see e.g. U.S. Pat. No. 9,273,283; U.S. Publication Nos. US2011/0158957, US2014/0301990, US2015/0098954, US2016/0208243, US2016/272999 and US2015/056705; International PCT Publication Nos. WO2014/191128, WO2015/136001, WO2015/161276, WO2016/069283, WO2016/016341, WO2017/193107, WO2017/093969, WO2019/070541, and WO2019/195492; and Osborn et al. (2016) Mol. Ther. 24(3):570-581.

In some of the embodiments provided herein, homology-directed repair (HDR) can be utilized for targeted integration of a specific portion of the template polynucleotide containing a transgene, e.g., nucleic acid sequence encoding any of the provided recombinant receptors, e.g., recombinant T cell receptor (TCR), at a particular location in the genome, e.g., the TRAC, TRBC1 and/or TRBC2 locus. In some embodiments, a template polynucleotide comprising a nucleic acid sequence, e.g., a transgene, encoding a recombinant T cell receptor (TCR) or antigen-binding fragment or chain thereof is introduced into a cell, e.g., an immune cell, having a genetic disruption of a target site within a T cell receptor alpha constant (TRAC) gene and/or a T cell receptor beta constant (TRBC) gene. In some embodiments, the nucleic acid sequence or transgene encoding the recombinant TCR or antigen-binding fragment or chain thereof is targeted for integration at or near the target site via homology directed repair (HDR). In particular embodiments, the integration at or near the target site is within a portion of coding sequence of a TRAC and/or TRBC gene, such as, for example, a portion of the coding sequence downstream of, or 3′ of the target site.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contain cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al., Blood. 1:72-82 (2012); Wang et al., J Immunother. 35(9): 689-701 (2012). In some embodiments, combining T_{CM}-enriched CD8⁺ T cells and CD4⁺ T cells further enhances efficacy.

In embodiments, memory T cells are present in both CD62L⁺ and CD62L-subsets of CD8⁺ peripheral blood lymphocytes. PBMC can be enriched for or depleted of CD62L-CD8⁺ and/or CD62L⁺ CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO−, CD45RA+, CD62L⁺, CD4⁺ T cells. In some embodiments, central memory CD4⁺ cells are CD62L⁺ and CD45RO⁺. In some embodiments, effector CD4⁺ cells are CD62L− and CD45RO−.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In vitro and In vivo, p 17-25 Edited by: S. A. Brooks and U. Schumacher © Humana Press Inc., Totowa, NJ).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, CA). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in PCT Pub. Number WO2009/072003, or US20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al., J Immunother. 35(9): 651-660 (2012), Terakura et al., Blood. 1:72-82 (2012), and Wang et al., J Immunother. 35(9): 689-701 (2012).

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al., Lab Chip 10, 1567-1573 (2010); and Godin et al., J Biophoton. 1(5): 355-376(2008). In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80 °C at a rate of 1° per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al., Klebanoff et al., J Immunother. 35(9): 651-660 (2012), Terakura et al., Blood. 1:72-82 (2012), and/or Wang et al., J Immunother. 35(9): 689-701 (2012).

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4⁺ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

### C. Nucleic Acids, Vectors and Methods for Genetic Engineering

In some embodiments, the cells, e.g., T cells, are genetically engineered to express a recombinant receptor. In some embodiments, the engineering is carried out by introducing nucleic acid molecules that encode the recombinant receptor. Also provided are nucleic acid molecules encoding a recombinant receptor, and vectors or constructs containing such nucleic acids and/or nucleic acid molecules.

In some cases, the nucleic acid sequence encoding the recombinant receptor, e.g., chimeric antigen receptor (CAR), contains a signal sequence that encodes a signal peptide. In some aspects, the signal sequence may encode a signal peptide derived from a native polypeptide. In other aspects, the signal sequence may encode a heterologous or non-native signal peptide. In some embodiments, the signal peptide is derived from a transmembrane protein. In some examples the signal peptide is derived from CD8a, CD33, or an IgG. Non-limiting exemplary examples of signal peptides include, for example, the CD33 signal peptide set forth in SEQ ID NO:21, CD8a signal peptide set forth in SEQ ID NO:75, or the signal peptide set forth in SEQ ID NO:76 or modified variant thereof.

In some embodiments, the nucleic acid molecule encoding the recombinant receptor contains at least one promoter that is operatively linked to control expression of the recombinant receptor. In some examples, the nucleic acid molecule contains two, three, or more promoters operatively linked to control expression of the recombinant receptor. In some embodiments, nucleic acid molecule can contain regulatory sequences, such as transcription and translation initiation and termination codons, which are specific to the type of host (e.g., bacterium, fungus, plant, or animal) into which the nucleic acid molecule is to be introduced, as appropriate and taking into consideration whether the nucleic acid molecule is DNA- or RNA-based. In some embodiments, the nucleic acid molecule can contain regulatory/control elements, such as a promoter, an enhancer, an intron, a polyadenylation signal, a Kozak consensus sequence, and splice acceptor or donor. In some embodiments, the nucleic acid molecule can contain a nonnative promoter operably linked to the nucleotide sequence encoding the recombinant receptor and/or one or more additional polypeptide(s). In some embodiments, the promoter is selected from among an RNA pol I, pol II or pol III promoter. In some embodiments, the promoter is recognized by RNA polymerase II (e.g., a CMV, SV40 early region or adenovirus major late promoter). In another embodiment, the promoter is recognized by RNA polymerase III (e.g., a U6 or H1 promoter). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1α promoter (EFla), mouse phosphoglycerate kinase 1 promoter (PGK), and chicken β-Actin promoter coupled with CMV early enhancer (CAGG). In some embodiments, the constitutive promoter is a synthetic or modified promoter. In some embodiments, the promoter is or comprises an MND promoter, a synthetic promoter that contains the U3 region of a modified MoMuLV LTR with myeloproliferative sarcoma virus enhancer (see Challita et al. (1995) J. Virol. 69(2): 748-755). In some embodiments, the promoter is a tissue-specific promoter. In another embodiment, the promoter is a viral promoter. In another embodiment, the promoter is a non-viral promoter.

In another embodiment, the promoter is a regulated promoter (e.g., inducible promoter). In some embodiments, the promoter is an inducible promoter or a repressible promoter. In some embodiments, the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof. In some embodiments, the nucleic acid molecule does not include a regulatory element, e.g. promoter.

In some embodiments, the nucleic acid molecule encoding the recombinant receptor, e.g., CAR or other antigen receptor, further includes nucleic acid sequences encoding a marker and/or cells expressing the CAR or other antigen receptor further includes a marker, e.g., a surrogate marker, such as a cell surface marker, which may be used to confirm transduction or engineering of the cell to express the receptor, such as a truncated version of a cell surface receptor, such as truncated EGFR (tEGFR). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the nucleic acid molecule, e.g., a nucleic acid molecule encoding a recombinant receptor. In some embodiments, the transduction marker can indicate or confirm modification of a cell. In some embodiments, the surrogate marker is a protein that is made to be co-expressed on the cell surface with the recombinant receptor, e.g. CAR. In particular embodiments, such a surrogate marker is a surface protein that has been modified to have little or no activity. In certain embodiments, the surrogate marker is encoded on the same nucleic acid molecule that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO:7 or 28) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR). In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some aspects, the marker, e.g. surrogate marker, includes all or part (e.g., truncated form) of CD34, a NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in PCT Pub. No. WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO: 7 or 28, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 28. An exemplary T2A linker sequence comprises the sequence of amino acids set forth in SEQ ID NO: 6 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, the sequence encodes a T2A ribosomal skip element set forth in SEQ ID NO: 6, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374). In some embodiments, the sequence encodes an EGFR sequence set forth in SEQ ID NO: 7 or 28, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 28.

In some embodiments, a single promoter may direct expression of an RNA that contains, in a single open reading frame (ORF), two or three genes (e.g. encoding the molecule involved in modulating a metabolic pathway and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13(2004) and deFelipe et al. Traffic 5:616-626(2004)). Many 2A elements are known in the art. Examples of 2A sequences that can be used in the methods and nucleic acids disclosed herein, without limitation, 2A sequences from the foot-and-mouth disease virus (F2A, e.g., SEQ ID NO: 27), equine rhinitis A virus (E2A, e.g., SEQ ID NO: 26), Thosea asigna virus (T2A, e.g., SEQ ID NO: 6 or 23), and porcine teschovirus-1 (P2A, e.g., SEQ ID NO: 24 or 25) as described in U.S. Patent Publication No. 20070116690.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

Introduction of the nucleic acid molecules encoding the recombinant receptor in the cell may be carried out using any of a number of known vectors. Such vectors include viral and non-viral systems, including lentiviral and gammaretroviral systems, as well as transposon-based systems such as PiggyBac or Sleeping Beauty-based gene transfer systems. Exemplary methods include those for transfer of nucleic acids encoding the receptors, including via viral, e.g., retroviral or lentiviral, transduction, transposons, and electroporation.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell 2:223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506:97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506:115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346:776-777(1990)); and strontium phosphate DNA co-precipitation (Brash et al., Mol. Cell Biol., 7:2031-2034(1987)).

Other approaches and vectors for transfer of the nucleic acids encoding the recombinant products are those described, e.g., in international patent application, Publication No.: WO2014055668, and U.S. Pat. No. 7,446,190.

In some embodiments, the cells, e.g., T cells, may be transfected either during or after expansion e.g. with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the CD3/CD28 stimulus, for example) and subsequently be stimulated with a second type of stimulus e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65:333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

In some aspects, the cells further are engineered to promote expression of cytokines or other factors. Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

### III. EXEMPLARY TREATMENT OUTCOMES AND METHODS FOR ASSESSING SAME

In some embodiments of the methods, compositions, combinations, uses, kits and articles of manufacture provided herein, the provided combination therapy results in one or more treatment outcomes, such as a feature associated with any one or more of the parameters associated with the therapy or treatment, as described below. In some embodiments, the method is any as described in Section I. In some embodiments, the method further includes assessment of the exposure, persistence and proliferation of the T cells, e.g., T cells administered for the T cell based therapy. In some embodiments, the exposure, or prolonged expansion and/or persistence of the cells, and/or changes in cell phenotypes or functional activity of the cells, e.g., cells administered for immunotherapy, e.g. T cell therapy, in the methods provided herein, can be measured by assessing the characteristics of the T cells in vitro or ex vivo. In some embodiments, such assays can be used to determine or confirm the function of the T cells, e.g. T cell therapy, before, during, or after administering the combination therapy provided herein.

In some embodiments, the combination therapy can further include one or more screening steps to identify subjects for treatment with the combination therapy and/or continuing the combination therapy, and/or a step for assessment of treatment outcomes and/or monitoring treatment outcomes. In some embodiments, the step for assessment of treatment outcomes can include steps to evaluate and/or to monitor treatment and/or to identify subjects for administration of further or remaining steps of the therapy and/or for repeat therapy. In some embodiments, the screening step and/or assessment of treatment outcomes can be used to determine the dose, frequency, duration, timing and/or order of the combination therapy provided herein.

In some embodiments, any of the screening steps and/or assessment of treatment of outcomes described herein can be used prior to, during, during the course of, or subsequent to administration of one or more steps of the provided combination therapy, e.g., administration of the T cell therapy (e.g. TCR- or CAR-expressing T cells), and/or administration of the DGK inhibitor. In some embodiments, assessment is made prior to, during, during the course of, or after performing any of the methods provided herein. In some embodiments, the assessment is made prior to performing the methods provided herein. In some embodiments, assessment is made after performing one or more steps of the methods provided herein. In some embodiments, the assessment is performed prior to administration of administration of one or more steps of the provided combination therapy, for example, to screen and identify patients suitable and/or susceptible to receive the combination therapy. In some embodiments, the assessment is performed during, during the course of, or subsequent to administration of one or more steps of the provided combination therapy, for example, to assess the intermediate or final treatment outcome, e.g., to determine the efficacy of the treatment and/or to determine whether to continue or repeat the treatments and/or to determine whether to administer the remaining steps of the combination therapy.

In some embodiments, treatment of outcomes includes improved immune function, e.g., immune function of the T cells administered for cell based therapy and/or of the endogenous T cells in the body. In some embodiments, exemplary treatment outcomes include, but are not limited to, enhanced T cell proliferation, enhanced T cell functional activity, changes in immune cell phenotypic marker expression, such as such features being associated with the engineered T cells, e.g. TCR or CAR-T cells, administered to the subject. In some embodiments, exemplary treatment outcomes include decreased disease burden, e.g., tumor burden, improved clinical outcomes and/or enhanced efficacy of therapy.

In some embodiments, the screening step and/or assessment of treatment of outcomes includes assessing the survival and/or function of the T cells administered for cell based therapy. In some embodiments, the screening step and/or assessment of treatment of outcomes includes assessing the levels of cytokines or growth factors. In some embodiments, the screening step and/or assessment of treatment of outcomes includes assessing disease burden and/or improvements, e.g., assessing tumor burden and/or clinical outcomes. In some embodiments, either of the screening step and/or assessment of treatment of outcomes can include any of the assessment methods and/or assays described herein and/or known, and can be performed one or more times, e.g., prior to, during, during the course of, or subsequently to administration of one or more steps of the combination therapy. Exemplary sets of parameters associated with a treatment outcome, which can be assessed in some embodiments of the methods provided herein, include peripheral blood immune cell population profile and/or tumor burden.

In some embodiments, the methods affect efficacy of the cell therapy in the subject. In some embodiments, the persistence, expansion, and/or presence of recombinant receptor-expressing, e.g., TCR- or CAR-expressing, cells in the subject following administration of the dose of cells in the method with a DGK inhibitor (e.g., any as described in Section I-B) is greater as compared to that achieved via a method without the administration of the DGK inhibitor. In some embodiments, expansion and/or persistence in the subject of the administered T cell therapy, e.g., TCR- or CAR-expressing T cells is assessed as compared to a method in which the T cell therapy is administered to the subject in the absence of a DGK inhibitor. In some embodiments, the methods result in the administered T cells exhibiting increased or prolonged expansion and/or persistence in the subject as compared to a method in which the T cell therapy is administered to the subject in the absence of a DGK inhibitor.

In some embodiments, the administration of a DGK inhibitor decreases disease burden, e.g., tumor burden, in the subject as compared to a method in which the dose of cells expressing the recombinant receptor is administered to the subject in the absence of a DGK inhibitor. In some embodiments, the administration of a DGK inhibitor decreases blast marrow in the subject as compared to a method in which the dose of cells expressing the recombinant receptor is administered to the subject in the absence of a DGK inhibitor. In some embodiments, the administration of a DGK inhibitor results in improved clinical outcomes, e.g., objective response rate (ORR), progression-free survival (PFS) and overall survival (OS), compared to a method in which the dose of cells expressing the recombinant receptor is administered to the subject in the absence of a DGK inhibitor.

In some embodiments, the subject can be screened prior to the administration of one or more steps of the combination therapy. For example, the subject can be screened for characteristics of the disease and/or disease burden, e.g., tumor burden, prior to administration of the combination therapy, to determine suitability, responsiveness and/or susceptibility to administering the combination therapy. In some embodiments, the screening step and/or assessment of treatment outcomes can be used to determine the dose, frequency, duration, timing and/or order of the combination therapy provided herein.

In some embodiments, the subject can be screened after administration of one of the steps of the combination therapy, to determine and identify subjects to receive the remaining steps of the combination therapy and/or to monitor efficacy of the therapy. In some embodiments, the number, level or amount of administered T cells and/or proliferation and/or activity of the administered T cells is assessed prior to administration and/or after administration of a DGK inhibitor.

In some embodiments, a change and/or an alteration, e.g., an increase, an elevation, a decrease or a reduction, in levels, values or measurements of a parameter or outcome compared to the levels, values or measurements of the same parameter or outcome in a different time point of assessment, a different condition, a reference point and/or a different subject is determined or assessed. For example, in some embodiments, a fold change, e.g., an increase or decrease, in particular parameters, e.g., number of engineered T cells in a sample, compared to the same parameter in a different condition, e.g., before administration of a DGK inhibitor can be determined. In some embodiments, the levels, values or measurements of two or more parameters are determined, and relative levels are compared. In some embodiments, the determined levels, values or measurements of parameters are compared to the levels, values or measurements from a control sample or an untreated sample. In some embodiments, the determined levels, values or measurements of parameters are compared to the levels from a sample from the same subject but at a different time point. The values obtained in the quantification of individual parameter can be combined for the purpose of disease assessment, e.g., by forming an arithmetical or logical operation on the levels, values or measurements of parameters by using multi-parametric analysis. In some embodiments, a ratio of two or more specific parameters can be calculated.

### A. T Cell Exposure, Persistence and Proliferation

In some embodiments, the parameter associated with therapy or a treatment outcome, which include parameters that can be assessed for the screening steps and/or assessment of treatment of outcomes and/or monitoring treatment outcomes, is or includes assessment of the exposure, persistence and proliferation of the T cells, e.g., T cells administered for the T cell based therapy. In some embodiments, the increased exposure, or prolonged expansion and/or persistence of the cells, and/or changes in cell phenotypes or functional activity of the cells, e.g., cells administered for immunotherapy, e.g. T cell therapy, in the methods provided herein, can be measured by assessing the characteristics of the T cells in vitro or ex vivo. In some embodiments, such assays can be used to determine or confirm the function of the T cells used for the immunotherapy, e.g. T cell therapy, before or after administering one or more steps of the combination therapy provided herein.

In some embodiments, the administration of the DGK inhibitor is designed to promote exposure of the subject to the cells, e.g., T cells administered for T cell based therapy, such as by promoting their expansion and/or persistence over time. In some embodiments, the T cell therapy exhibits increased or prolonged expansion and/or persistence in the subject as compared to a method in which the T cell therapy is administered to the subject in the absence of the DGK inhibitor.

In some embodiments, the provided methods increase exposure of the subject to the administered cells (e.g., increased number of cells or duration over time) and/or improve efficacy and therapeutic outcomes of the immunotherapy, e.g. T cell therapy. In some aspects, the methods are advantageous in that a greater and/or longer degree of exposure to the cells expressing the recombinant receptors, e.g., TCR- or CAR-expressing cells, improves treatment outcomes as compared with other methods. Such outcomes may include patient survival and remission, even in individuals with severe tumor burden.

In some embodiments, the administration of the DGK inhibitor can increase the maximum, total, and/or duration of exposure to the cells, e.g. T cells administered for the T cell based therapy, in the subject as compared to administration of the T cells alone in the absence of the DGK inhibitor.

In some embodiments, the presence and/or amount of cells expressing the recombinant receptor (e.g., TCR- or CAR-expressing cells administered for T cell based therapy) in the subject following the administration of the T cells and before, during and/or after the administration of the DGK inhibitor is detected. In some aspects, quantitative PCR (qPCR) is used to assess the quantity of cells expressing the recombinant receptor (e.g., TCR- or CAR-expressing cells administered for T cell based therapy) in the blood or serum or organ or tissue sample (e.g., disease site, e.g., tumor sample) of the subject. In some aspects, persistence is quantified as copies of DNA or plasmid encoding the receptor, e.g., TCR or CAR, per microgram of DNA, e.g., total DNA obtained from a sample, or as the number of receptor-expressing, e.g., TCR- or CAR-expressing, cells per microliter of the sample, e.g., of blood or serum, or per total number of peripheral blood mononuclear cells (PBMCs) or white blood cells or T cells per microliter of the sample.

In some embodiments, the cells are detected in the subject at or at least at 4, 7, 10, 14, 18, 21, 24, 27, or 28 days following the administration of the T cells, e.g., TCR- or CAR-expressing T cells. In some aspects, the cells are detected at or at least at 2, 4, or 6 weeks following, or 3, 6, or 12, 18, or 24, or 30 or 36 months, or 1, 2, 3, 4, 5, or more years, following the administration of the T cells.

In some embodiments, the persistence of receptor-expressing cells (e.g. TCR- or CAR-expressing cells) in the subject by the methods, following the administration of the T cells, e.g., TCR- or CAR-expressing T cells and/or the DGK inhibitor, is greater as compared to that which would be achieved by alternative methods such as those involving the administration of the immunotherapy alone, e.g., administration the T cells, e.g., TCR- or CAR-expressing T cells, in the absence of the DGK inhibitor.

The exposure, e.g., number of cells, e.g. T cells administered for T cell therapy, indicative of expansion and/or persistence, may be stated in terms of maximum numbers of the cells to which the subject is exposed, duration of detectable cells or cells above a certain number or percentage, area under the curve for number of cells over time, and/or combinations thereof and indicators thereof. Such outcomes may be assessed using known methods, such as qPCR to detect copy number of nucleic acid encoding the recombinant receptor compared to total amount of nucleic acid or DNA in the particular sample, e.g., blood, serum, plasma or tissue, such as a tumor sample, and/or flow cytometric assays detecting cells expressing the receptor generally using antibodies specific for the receptors. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor.

In some aspects, increased exposure of the subject to the cells includes increased expansion of the cells. In some embodiments, the receptor expressing cells, e.g. TCR- or CAR-expressing cells, expand in the subject following administration of the T cells, e.g., TCR- or CAR-expressing T cells, and/or following administration of the DGK inhibitor. In some aspects, the methods result in greater expansion of the cells compared with other methods, such as those involving the administration of the T cells, e.g., TCR- or CAR-expressing T cells, in the absence of administering the DGK inhibitor.

In some aspects, the method results in high in vivo proliferation of the administered cells, for example, as measured by flow cytometry. In some aspects, high peak proportions of the cells are detected. For example, in some embodiments, at a peak or maximum level following the administration of the T cells, e.g., TCR- or CAR-expressing T cells and/or the DGK inhibitor, in the blood or disease-site of the subject or white blood cell fraction thereof, e.g., PBMC fraction or T cell fraction, at least about 10%, at least about 20%, at least about 30%, at least about 40%, at least about 50%, at least about 60%, at least about 70%, at least about 80%, or at least about 90% of the cells express the recombinant receptor, e.g., the TCR or CAR.

In some embodiments, the method results in a maximum concentration, in the blood or serum or other bodily fluid or organ or tissue of the subject, of at least 100, 500, 1000, 1500, 2000, 5000, 10,000 or 15,000 copies of or nucleic acid encoding the receptor, e.g., the TCR or CAR, per microgram of DNA, or at least 0.1, 0.2, 0.3, 0.4, 0.5, 0.6, 0.7, 0.8, or 0.9 receptor-expressing, e.g., TCR- or CAR-expressing cells per total number of peripheral blood mononuclear cells (PBMCs), total number of mononuclear cells, total number of T cells, or total number of microliters. In some embodiments, the cells expressing the receptor are detected as at least 10, 20, 30, 40, 50, or 60% of total PBMCs in the blood of the subject, and/or at such a level for at least 1, 2, 3, 4, 5, 6, 7, 8, 9, 10, 11, 12, 24, 36, 48, or 52 weeks following the T cells, e.g., TCR- or CAR-expressing T cells and/or the DGK inhibitor, or for 1, 2, 3, 4, or 5, or more years following such administration.

In some aspects, the method results in at least a 2-fold, at least a 4-fold, at least a 10-fold, or at least a 20-fold increase in copies of nucleic acid encoding the recombinant receptor, e.g., TCR or CAR, per microgram of DNA, e.g., in the serum, plasma, blood or tissue, e.g., tumor sample, of the subject.

In some embodiments, cells expressing the receptor are detectable in the serum, plasma, blood or tissue, e.g., tumor sample, of the subject, e.g., by a specified method, such as qPCR or flow cytometry-based detection method, at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 or more days following administration of the T cells, e.g., TCR- or CAR-expressing T cells, or after administration of the DGK inhibitor, for at least at or about 2, 3, 4, 5, 6, 7, 8, 9, 10, 11, 12, 13, 14, 15, 16, 17, 18, 19, 20, 21, 22, 23, or 24 or more weeks following the administration of the T cells, e.g., TCR- or CAR-expressing T cells, and/or the DGK inhibitor.

In some aspects, at least about 1×10², at least about 1×10³, at least about 1×10⁴, at least about 1×10⁵, or at least about 1×10⁶ or at least about 5×10⁶ or at least about 1×10⁷ or at least about 5×10⁷ or at least about 1×10⁸ recombinant receptor-expressing, e.g., TCR- or CAR-expressing cells, and/or at least 10, 25, 50, 100, 200, 300, 400, or 500, or 1000 receptor-expressing cells per microliter, e.g., at least 10 per microliter, are detectable or are present in the subject or fluid, plasma, serum, tissue, or compartment thereof, such as in the blood, e.g., peripheral blood, or disease site, e.g., tumor, thereof. In some embodiments, such a number or concentration of cells is detectable in the subject for at least about 20 days, at least about 40 days, or at least about 60 days, or at least about 3, 4, 5, 6, 7, 8, 9, 10, 11, or 12 months, or at least 2 or 3 years, following administration of the T cells, e.g., TCR- or CAR-expressing T cells, and/or following the administration of the DGK inhibitor. Such cell numbers may be as detected by flow cytometry-based or quantitative PCR-based methods and extrapolation to total cell numbers using known methods. See, e.g., Brentjens et al., Sci Transl Med. 2013 5(177), Park et al, Molecular Therapy 15(4): 825-833(2007), Savoldo et al., JCI 121(5): 1822-1826(2011), Davila et al., (2013) PLoS ONE 8(4):e61338, Davila et al., Oncoimmunology 1(9): 1577-1583 (2012), Lamers, Blood 2011 117:72-82, Jensen et al., Biol Blood Marrow Transplant 2010 September; 16(9): 1245-1256, Brentjens et al., Blood 2011 118(18): 4817-4828.

In some aspects, the copy number of nucleic acid encoding the recombinant receptor, e.g., vector copy number, per 100 cells, for example in the peripheral blood or bone marrow or other compartment, as measured by immunohistochemistry, PCR, and/or flow cytometry, is at least 0.01, at least 0.1, at least 1, or at least 10, at about 1 week, about 2 weeks, about 3 weeks, about 4 weeks, about 5 weeks, or at least about 6 weeks, or at least about 2, 3, 4, 5, 6, 7, 8. 9, 10, 11, or 12 months or at least 2 or 3 years following administration of the cells, e.g., TCR- or CAR-expressing T cells, and/or the DGK inhibitor. In some embodiments, the copy number of the vector expressing the receptor, e.g. TCR or CAR, per microgram of genomic DNA is at least 100, at least 1000, at least 5000, or at least 10,000, or at least 15,000 or at least 20,000 at a time about 1 week, about 2 weeks, about 3 weeks, or at least about 4 weeks following administration of the T cells, e.g., TCR- or CAR-expressing T cells, or the DGK inhibitor, or at least 2, 3, 4, 5, 6, 7, 8, 9, 10, 11, or 12 months or at least 2 or 3 years following such administration.

In some aspects, the receptor, e.g. TCR or CAR, expressed by the cells, is detectable by quantitative PCR (qPCR) or by flow cytometry in the subject, plasma, serum, blood, tissue and/or disease site thereof, e.g., tumor site, at a time that is at least about 3 months, at least about 6 months, at least about 12 months, at least about 1 year, at least about 2 years, at least about 3 years, or more than 3 years, following the administration of the cells, e.g., following the initiation of the administration of the T cells, e.g., TCR- or CAR-expressing T cells, and/or the DGK inhibitor.

In some embodiments, the area under the curve (AUC) for concentration of receptor- (e.g., TCR- or CAR-) expressing cells in a fluid, plasma, serum, blood, tissue, organ and/or disease site, e.g. tumor site, of the subject over time following the administration of the T cells, e.g., TCR- or CAR-expressing T cells and/or the DGK inhibitor, is greater as compared to that achieved via an alternative dosing regimen where the subject is administered the T cells, e.g., TCR- or CAR-expressing T cells, in the absence of administering the DGK inhibitor.

In some aspects, the method results in high in vivo proliferation of the administered cells, for example, as measured by flow cytometry. In some aspects, high peak proportions of the cells are detected. For example, in some embodiments, at a peak or maximum level following the T cells, e.g., TCR- or CAR-expressing T cells and/or the DGK inhibitor, in the blood, plasma, serum, tissue or disease site of the subject or white blood cell fraction thereof, e.g., PBMC fraction or T cell fraction, at least about 10%, at least about 20%, at least about 30%, at least about 40%, at least about 50%, at least about 60%, at least about 70%, at least about 80%, or at least about 90% of the cells express the recombinant receptor, e.g., the TCR or CAR.

In some aspects, the increased or prolonged expansion and/or persistence of the dose of cells in the subject administered with the DGK inhibitor is associated with a benefit in tumor related outcomes in the subject. In some embodiments, the tumor related outcome includes a decrease in tumor burden or a decrease in blast marrow in the subject. In some embodiments, the tumor burden is decreased by or by at least at or about 10, 20, 30, 40, 50, 60, 70, 80, 90, or 100 percent after administration of the method. In some embodiments, disease burden, tumor size, tumor volume, tumor mass, and/or tumor load or bulk is reduced following the dose of cells by at least at or about 50%, 60%, 70%, 80%, 90% or more compared a subject that has been treated with a method that does not involve the administration of the DGK inhibitor.

### B. T Cell Functional Activity

In some embodiments, parameters associated with therapy or a treatment outcome, which include parameters that can be assessed for the screening steps and/or assessment of treatment of outcomes and/or monitoring treatment outcomes, includes one or more of activity, phenotype, proliferation or function of T cells. In some embodiments, any of the known assays in the art for assessing the activity, phenotypes, proliferation and/or function of the T cells, e.g., T cells administered for T cell therapy, can be used. Prior to and/or subsequent to administration of the cells and/or the DGK inhibitor, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known such as cytotoxicity assays described in, for example, Kochenderfer et al., J. Immunotherapy, 32(7): 689-702 (2009), and Herman et al., J. Immunological Methods, 285(1): 25-40 (2004).

In some embodiments, T cells, such as recombinant-expressing (e.g., TCR or CAR) T cells, can be assessed prior to and/or subsequent to administration of the cells and/or the DGK inhibitor to assess or determine if the T cells exhibit features of exhaustion. In some cases, exhaustion can be assessed by monitoring loss of T cell function, such as reduced or decreased antigen-specific or antigen receptor-driven activity, such as a reduced or decreased ability to produce cytokines or to drive cytolytic activity against target antigen. In some cases, exhaustion also can be assessed by monitoring expression of surface markers on T cells (e.g. CD4 and/or CD4 T cells) that are associated with an exhaustion phenotype. Among exhaustion markers are inhibitory receptors such as PD-1, CTLA-4, LAG-3 and TIM-3.

In some embodiments, such a reduced or decreased activity is observed over time following administration to the subject and/or following long-term exposure to antigen.

In particular embodiments, the provided methods (i) to effect said increase in antigen-specific or antigen receptor-driven activity and (ii) to prevent, inhibit or delay said onset of exhaustion phenotype and/or to reverse said exhaustion phenotype. In some embodiments, the amount, duration and/or frequency is effective (i) to effect said increase in antigen-specific or antigen receptor-driven activity and (ii) to prevent, inhibit or delay said onset of exhaustion phenotype. In other embodiments, the amount, duration and/or frequency is effective (i) to effect said increase in antigen-specific or antigen receptor-driven activity and (ii) to prevent, inhibit or delay said onset of exhaustion phenotype and to reverse said exhaustion phenotype.

In some embodiments, the exhaustion phenotype, with reference to a T cell or population of T cells, includes: an increase in the level or degree of surface expression on the T cell or T cells, or in the percentage of said population of T cells exhibiting surface expression, of one or more exhaustion marker, optionally 2, 3, 4, 5 or 6 exhaustion markers, compared to a reference T cell population under the same conditions; or a decrease in the level or degree of an activity exhibited by said T cells or population of T cells upon exposure to an antigen or antigen receptor-specific agent, compared to a reference T cell population, under the same conditions. In some embodiments, the increase in the level, degree or percentage is by greater than at or about 1.2-fold, at or about 1.5-fold, at or about 2.0-fold, at or about 3-fold, at or about 4-fold, at or about 5-fold, at or about 6-fold, at or about 7-fold, at or about 8-fold, at or about 9-fold, at or about 10-fold or more. In other embodiments, the decrease in the level, degree or percentage is by greater than at or about 1.2-fold, at or about 1.5-fold, at or about 2.0-fold, at or about 3-fold, at or about 4-fold, at or about 5-fold, at or about 6-fold, at or about 7-fold, at or about 8-fold, at or about 9-fold, at or about 10-fold or more.

In certain embodiments, the biological activity of the cells is measured by assaying expression and/or secretion of one or more cytokines, such as CD107a, IFNγ, IL-2, GM-CSF and TNFα, and/or by assessing cytolytic activity.

In some embodiments, assays for the activity, phenotypes, proliferation and/or function of the T cells, e.g., T cells administered for T cell therapy include, but are not limited to, ELISPOT, ELISA, cellular proliferation, cytotoxic lymphocyte (CTL) assay, binding to the T cell epitope, antigen or ligand, or intracellular cytokine staining, proliferation assays, lymphokine secretion assays, direct cytotoxicity assays, and limiting dilution assays. In some embodiments, proliferative responses of the T cells can be measured, e.g. by incorporation of ³H-thymidine, BrdU (5-Bromo-2′-Deoxyuridine) or 2′-deoxy-5-ethynyluridine (EdU) into their DNA or dye dilution assays, using dyes such as carboxyfluorescein diacetate succinimidyl ester (CFSE), CellTrace Violet, or membrane dye PKH26.

In some embodiments, assessing the activity, phenotypes, proliferation and/or function of the T cells, e.g., T cells administered for T cell therapy, include measuring cytokine production from T cells, and/or measuring cytokine production in a biological sample from the subject, e.g., plasma, serum, blood, and/or tissue samples, e.g., tumor samples. In some cases, such measured cytokines can include, without limitation, interlekukin-2 (IL-2), interferon-gamma (IFNγ), interleukin-4 (IL-4), TNF-alpha (TNFα), interleukin-6 (IL-6), interleukin-10 (IL-10), interleukin-12 (IL-12), granulocyte-macrophage colony-stimulating factor (GM-CSF), CD107a, and/or TGF-beta (TGFβ). Assays to measure cytokines are well known, and include but are not limited to, ELISA, intracellular cytokine staining, cytometric bead array, RT-PCR, ELISPOT, flow cytometry and bio-assays in which cells responsive to the relevant cytokine are tested for responsiveness (e.g. proliferation) in the presence of a test sample.

In some embodiments, assessing the activity, phenotypes, proliferation and/or function of the T cells, e.g., T cells administered for T cell therapy, include assessing cell phenotypes, e.g., expression of particular cell surface markers. In some embodiments, the T cells, e.g., T cells administered for T cell therapy, are assessed for expression of T cell activation markers, T cell exhaustion markers, and/or T cell differentiation markers. In some embodiments, the cell phenotype is assessed before administration. In some embodiments, the cell phenotype is assessed during, or after administration of cell therapy and/or the DGK inhibitor. T cell activation markers, T cell exhaustion markers, and/or T cell differentiation markers for assessment include any markers known for particular subsets of T cells, e.g., CD25, CD38, human leukocyte antigen-DR (HLA-DR), CD69, CD44, CD137, KLRG1, CD62L^{low}, CCR7^{low}, CD71, CD2, CD54, CD58, CD244, CD160, programmed cell death protein 1 (PD-1), lymphocyte activation gene 3 protein (LAG-3), T-cell immunoglobulin domain and mucin domain protein 3 (TIM-3), cytotoxic T lymphocyte antigen-4 (CTLA-4), band T lymphocyte attenuator (BTLA) and/or T-cell immunoglobulin and immunoreceptor tyrosine-based inhibitory motif domain (TIGIT) (see, e.g., Liu et al., Cell Death and Disease (2015) 6, e1792). In some embodiments, the exhaustion marker is any one or more of PD-1, CTLA-4, TIM-3, LAG-3, BTLA, 2B4, CD160, CD39, VISTA, and TIGIT In some embodiments, the assessed cell surface marker is CD25, PD-1 and/or TIM-3. In some embodiments, the assessed cell surface marker is CD25.

In some aspects, detecting the expression levels includes performing an in vitro assay. In some embodiments, the in vitro assay is an immunoassay, an aptamer-based assay, a histological or cytological assay, or an mRNA expression level assay. In some embodiments, the parameter or parameters for one or more of each of the one or more factors, effectors, enzymes and/or surface markers are detected by an enzyme linked immunosorbent assay (ELISA), immunoblotting, immunoprecipitation, radioimmunoassay (RIA), immunostaining, flow cytometry assay, surface plasmon resonance (SPR), chemiluminescence assay, lateral flow immunoassay, inhibition assay or avidity assay. In some embodiments, detection of cytokines and/or surface markers is determined using a binding reagent that specifically binds to at least one biomarker. In some cases, the binding reagent is an antibody or antigen-binding fragment thereof, an aptamer or a nucleic acid probe.

In some embodiments, the administration of the DGK inhibitor increases the level of circulating CAR T cells.

### C. Disease Burden

In some embodiments, parameters associated with therapy or a treatment outcome, which include parameters that can be assessed for the screening steps and/or assessment of treatment of outcomes and/or monitoring treatment outcomes, includes tumor or disease burden. The administration of the immunotherapy, such as a T cell therapy (e.g. TCR- or CAR-expressing T cells) and/or the DGK inhibitor, can reduce or prevent the expansion or burden of the disease or condition in the subject. For example, where the disease or condition is a tumor, the methods generally reduce tumor size, bulk, metastasis, percentage of blasts in the bone marrow or molecularly detectable B cell malignancy and/or improve prognosis or survival or other symptom associated with tumor burden.

In some aspects, the administration in accord with the provided methods, and/or with the provided articles of manufacture or compositions, generally reduces or prevents the expansion or burden of the disease or condition in the subject. For example, where the disease or condition is a tumor, the methods generally reduce tumor size, bulk, metastasis, percentage of blasts in the bone marrow or molecularly detectable B cell malignancy and/or improve prognosis or survival or other symptom associated with tumor burden.

In some embodiments, the provided methods result in a decreased tumor burden in treated subjects compared to alternative methods in which the immunotherapy, such as a T cell therapy (e.g. TCR- or CAR-expressing T cells) is given without administration of the DGK inhibitor. It is not necessary that the tumor burden actually be reduced in all subjects receiving the combination therapy, but that tumor burden is reduced on average in subjects treated, such as based on clinical data, in which a majority of subjects treated with such a combination therapy exhibit a reduced tumor burden, such as at least 50%, 60%, 70%, 80%, 90%, 95% or more of subjects treated with the combination therapy, exhibit a reduced tumor burden.

Disease burden can encompass a total number of cells of the disease in the subject or in an organ, tissue, or bodily fluid of the subject, such as the organ or tissue of the tumor or another location, e.g., which would indicate metastasis. For example, tumor cells may be detected and/or quantified in the blood, lymph or bone marrow in the context of certain hematological malignancies. Disease burden can include, in some embodiments, the mass of a tumor, the number or extent of metastases and/or the percentage of blast cells present in the bone marrow.

In some embodiments, the subject has a lymphoma or a leukemia. The extent of disease burden can be determined by assessment of residual leukemia in blood or bone marrow. In some embodiments, the subject has a non-Hodgkin lymphoma (NHL), an acute lymphoblastic leukemia (ALL), a chronic lymphocytic leukemia (CLL), or a diffuse large B-cell lymphoma (DLBCL). In some embodiments, the subject has a MM or a DBCBL.

In some aspects, response rates in subjects, such as subjects with NHL, are based on the Lugano criteria. (Cheson et al., (2014) JCO., 32(27): 3059-3067; Johnson et al., (2015) Radiology 2:323-338; Cheson, B. D. (2015) Chin. Clin. Oncol. 4(1): 5). In some aspects, response assessment utilizes any of clinical, hematologic, and/or molecular methods. In some aspects, response assessed using the Lugano criteria involves the use of positron emission tomography (PET)-computed tomography (CT) and/or CT as appropriate. PET-CT evaluations may further comprise the use of fluorodeoxyglucose (FDG) for FDG-avid lymphomas. In some aspects, where PET-CT will be used to assess response in FDG-avid histologies, a 5-point scale may be used. In some respects, the 5-point scale comprises the following criteria: 1, no uptake above background; 2, uptake≤mediastinum; 3, uptake>mediastinum but≤liver; 4, uptake moderately>liver; 5, uptake markedly higher than liver and/or new lesions; X, new areas of uptake unlikely to be related to lymphoma.

In some aspects, a complete response as described using the Lugano criteria involves a complete metabolic response and a complete radiologic response at various measureable sites. In some aspects, these sites include lymph nodes and extralymphatic sites, wherein a CR is described as a score of 1, 2, or 3 with or without a residual mass on the 5-point scale, when PET-CT is used. In some aspects, in Waldeyer's ring or extranodal sites with high physiologic uptake or with activation within spleen or marrow (e.g., with chemotherapy or myeloid colony-stimulating factors), uptake may be greater than normal mediastinum and/or liver. In this circumstance, complete metabolic response may be inferred if uptake at sites of initial involvement is no greater than surrounding normal tissue even if the tissue has high physiologic uptake. In some aspects, response is assessed in the lymph nodes using CT, wherein a CR is described as no extralymphatic sites of disease and target nodes/nodal masses must regress to ≤1.5 cm in longest transverse diameter of a lesion (LDi). Further sites of assessment include the bone marrow wherein PET-CT-based assessment should indicate a lack of evidence of FDG-avid disease in marrow and a CT-based assessment should indicate a normal morphology, which if indeterminate should be IHC negative. Further sites may include assessment of organ enlargement, which should regress to normal. In some aspects, nonmeasured lesions and new lesions are assessed, which in the case of CR should be absent (Cheson et al., (2014) JCO., 32(27): 3059-3067; Johnson et al., (2015) Radiology 2:323-338; Cheson, B. D. (2015) Chin. Clin. Oncol. 4(1):5).

In some aspects, a partial response (PR) as described using the Lugano criteria involves a partial metabolic and/or radiological response at various measureable sites. In some aspects, these sites include lymph nodes and extralymphatic sites, wherein a PR is described as a score of 4 or 5 with reduced uptake compared with baseline and residual mass(es) of any size, when PET-CT is used. At interim, such findings can indicate responding disease. At the end of treatment, such findings can indicate residual disease. In some aspects, response is assessed in the lymph nodes using CT, wherein a PR is described as ≥50% decrease in SPD of up to 6 target measureable nodes and extranodal sites. If a lesion is too small to measure on CT, 5 mm×5 mm is assigned as the default value; if the lesion is no longer visible, the value is 0 mm×0 mm; for a node >5 mm×5 mm, but smaller than normal, actual measurements are used for calculation. Further sites of assessment include the bone marrow wherein PET-CT-based assessment should indicate residual uptake higher than uptake in normal marrow but reduced compared with baseline (diffuse uptake compatible with reactive changes from chemotherapy allowed). In some aspects, if there are persistent focal changes in the marrow in the context of a nodal response, consideration should be given to further evaluation with MRI or biopsy, or an interval scan. In some aspects, further sites may include assessment of organ enlargement, where the spleen must have regressed by >50% in length beyond normal. In some aspects, nonmeasured lesions and new lesions are assessed, which in the case of PR should be absent/normal, regressed, but no increase. No response/stable disease (SD) or progressive disease (PD) can also be measured using PET-CT and/or CT based assessments. (Cheson et al., (2014) JCO., 32(27): 3059-3067; Johnson et al., (2015) Radiology 2:323-338; Cheson, B. D. (2015) Chin. Clin. Oncol., 4(1): 5).

In some respects, progression-free survival (PFS) is described as the length of time during and after the treatment of a disease, such as a B cell malignancy, that a subject lives with the disease but it does not get worse. In some aspects, objective response (OR) is described as a measurable response. In some aspects, objective response rate (ORR) is described as the proportion of patients who achieved CR or PR. In some aspects, overall survival (OS) is described as the length of time from either the date of diagnosis or the start of treatment for a disease, such as a B cell malignancy, that subjects diagnosed with the disease are still alive. In some aspects, event-free survival (EFS) is described as the length of time after treatment for a B cell malignancy ends that the subject remains free of certain complications or events that the treatment was intended to prevent or delay. These events may include the return of the B cell malignancy or the onset of certain symptoms, such as bone pain from B cell malignancy that has spread to the bone, or death.

In some embodiments, the measure of duration of response (DOR) includes the time from documentation of tumor response to disease progression. In some embodiments, the parameter for assessing response can include durable response, e.g., response that persists after a period of time from initiation of therapy. In some embodiments, durable response is indicated by the response rate at approximately 1, 2, 3, 4, 5, 6, 7, 8, 9, 10, 11, 12, 18 or 24 months after initiation of therapy. In some embodiments, the response is durable for greater than 3 months or greater than 6 months.

In some aspects, the RECIST criteria is used to determine objective tumor response. (Eisenhauer et al., European Journal of Cancer 45(2009) 228-247.) In some aspects, the RECIST criteria is used to determine objective tumor response for target lesions. In some respects, a complete response as determined using RECIST criteria is described as the disappearance of all target lesions and any pathological lymph nodes (whether target or non-target) must have reduction in short axis to <10 mm. In other aspects, a partial response as determined using RECIST criteria is described as at least a 30% decrease in the sum of diameters of target lesions, taking as reference the baseline sum diameters. In other aspects, progressive disease (PD) is described as at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm (in some aspects the appearance of one or more new lesions is also considered progression). In other aspects, stable disease (SD) is described as neither sufficient shrinkage to qualify for PR nor sufficient increase to qualify for PD, taking as reference the smallest sum diameters while on study.

In the case of MM, exemplary parameters to assess the extent of disease burden include such parameters as number of clonal plasma cells (e.g., >10% on bone marrow biopsy or in any quantity in a biopsy from other tissues; plasmacytoma), presence of monoclonal protein (paraprotein) in either serum or urine, evidence of end-organ damage felt related to the plasma cell disorder (e.g., hypercalcemia (corrected calcium >2.75 mmol/l); renal insufficiency attributable to myeloma; anemia (hemoglobin <10 g/dl); and/or bone lesions (lytic lesions or osteoporosis with compression fractures)).

In the case of DLBCL, exemplary parameters to assess the extent of disease burden include such parameters as cellular morphology (e.g., centroblastic, immunoblastic, and anaplastic cells), gene expression, miRNA expression and protein expression (e.g., expression of BCL2, BCL6, MUM1, LMO2, MYC, and p21).

In some aspects, response rates in subjects, such as subjects with CLL, are based on the International Workshop on Chronic Lymphocytic Leukemia (IWCLL) response criteria (Hallek, et al., Blood 2008 June 15; 111(12): 5446-5456). In some aspects, these criteria are described as follows: complete remission (CR), which in some aspects requires the absence of peripheral blood clonal lymphocytes by immunophenotyping, absence of lymphadenopathy, absence of hepatomegaly or splenomegaly, absence of constitutional symptoms and satisfactory blood counts; complete remission with incomplete marrow recovery (CRi), which in some aspects is described as CR above, but without normal blood counts; partial remission (PR), which in some aspects is described as ≥50% fall in lymphocyte count, ≥50% reduction in lymphadenopathy or ≥50% reduction in liver or spleen, together with improvement in peripheral blood counts; progressive disease (PD), which in some aspects is described as ≥50% rise in lymphocyte count to >5×10⁹/L, ≥50% increase in lymphadenopathy, ≥50% increase in liver or spleen size, Richter's transformation, or new cytopenias due to CLL; and stable disease, which in some aspects is described as not meeting criteria for CR, CRi, PR or PD.

In some embodiments, the subjects exhibits a CR or OR if, within 1 month of the administration of the dose of cells, lymph nodes in the subject are less than at or about 20 mm in size, less than at or about 10 mm in size or less than at or about 10 mm in size.

In some embodiments, an index clone of the CLL is not detected in the bone marrow of the subject (or in the bone marrow of greater than 50%, 60%, 70%, 80%, 90% or more of the subjects treated according to the methods. In some embodiments, an index clone of the CLL is assessed by IgH deep sequencing. In some embodiments, the index clone is not detected at a time that is at or about or at least at or about 1, 2, 3, 4, 5, 6, 12, 18 or 24 months following the administration of the cells.

In some embodiments, a subject exhibits morphologic disease if there are greater than or equal to 5% blasts in the bone marrow, for example, as detected by light microscopy, such as greater than or equal to 10% blasts in the bone marrow, greater than or equal to 20% blasts in the bone marrow, greater than or equal to 30% blasts in the bone marrow, greater than or equal to 40% blasts in the bone marrow or greater than or equal to 50% blasts in the bone marrow. In some embodiments, a subject exhibits complete or clinical remission if there are less than 5% blasts in the bone marrow.

In some embodiments, a subject may exhibit complete remission, but a small proportion of morphologically undetectable (by light microscopy techniques) residual leukemic cells are present. A subject is said to exhibit minimum residual disease (MRD) if the subject exhibits less than 5% blasts in the bone marrow and exhibits molecularly detectable B cell malignancy. In some embodiments, molecularly detectable B cell malignancy can be assessed using any of a variety of molecular techniques that permit sensitive detection of a small number of cells. In some aspects, such techniques include PCR assays, which can determine unique Ig/T-cell receptor gene rearrangements or fusion transcripts produced by chromosome translocations. In some embodiments, flow cytometry can be used to identify B cell malignancy cell based on leukemia-specific immunophenotypes. In some embodiments, molecular detection of B cell malignancy can detect as few as 1 leukemia cell in 100,000 normal cells. In some embodiments, a subject exhibits MRD that is molecularly detectable if at least or greater than 1 leukemia cell in 100,000 cells is detected, such as by PCR or flow cytometry. In some embodiments, the disease burden of a subject is molecularly undetectable or MRD″, such that, in some cases, no leukemia cells are able to be detected in the subject using PCR or flow cytometry techniques.

In the case of leukemia, the extent of disease burden can be determined by assessment of residual leukemia in blood or bone marrow. In some embodiments, a subject exhibits morphologic disease if there are greater than or equal to 5% blasts in the bone marrow, for example, as detected by light microscopy. In some embodiments, a subject exhibits complete or clinical remission if there are less than 5% blasts in the bone marrow.

In some embodiments, for leukemia, a subject may exhibit complete remission, but a small proportion of morphologically undetectable (by light microscopy techniques) residual leukemic cells are present. A subject is said to exhibit minimum residual disease (MRD) if the subject exhibits less than 5% blasts in the bone marrow and exhibits molecularly detectable B cell malignancy. In some embodiments, molecularly detectable B cell malignancy can be assessed using any of a variety of molecular techniques that permit sensitive detection of a small number of cells. In some aspects, such techniques include PCR assays, which can determine unique Ig/T-cell receptor gene rearrangements or fusion transcripts produced by chromosome translocations. In some embodiments, flow cytometry can be used to identify B cell malignancy cell based on leukemia-specific immunophenotypes. In some embodiments, molecular detection of B cell malignancy can detect as few as 1 leukemia cell in 100,000 normal cells. In some embodiments, a subject exhibits MRD that is molecularly detectable if at least or greater than 1 leukemia cell in 100,000 cells is detected, such as by PCR or flow cytometry. In some embodiments, the disease burden of a subject is molecularly undetectable or MRD⁻, such that, in some cases, no leukemia cells are able to be detected in the subject using PCR or flow cytometry techniques.

In some embodiments, the methods and/or administration of a cell therapy, such as a T cell therapy (e.g. TCR- or CAR-expressing T cells) and/or the DGK inhibitor decrease(s) disease burden as compared with disease burden at a time immediately prior to the administration of the immunotherapy, e.g., T cell therapy and/or the DGK inhibitor.

In some aspects, administration of the immunotherapy, e.g. T cell therapy and/or the DGK inhibitor, may prevent an increase in disease burden, and this may be evidenced by no change in disease burden.

In some embodiments, the method reduces the burden of the disease or condition, e.g., number of tumor cells, size of tumor, duration of patient survival or event-free survival, to a greater degree and/or for a greater period of time as compared to the reduction that would be observed with a comparable method using an alternative therapy, such as one in which the subject receives immunotherapy, e.g. T cell therapy alone, in the absence of administration of the DGK inhibitor. In some embodiments, disease burden is reduced to a greater extent or for a greater duration following the combination therapy of administration of the immunotherapy, e.g., T cell therapy, and the DGK inhibitor, compared to the reduction that would be effected by administering each of the agent alone, e.g., administering the DGK inhibitorto a subject having not received the immunotherapy, e.g. T cell therapy; or administering the immunotherapy, e.g. T cell therapy, to a subject having not received the DGK inhibitor.

In some embodiments, the burden of a disease or condition in the subject is detected, assessed, or measured. Disease burden may be detected in some aspects by detecting the total number of disease or disease-associated cells, e.g., tumor cells, in the subject, or in an organ, tissue, or bodily fluid of the subject, such as blood or serum. In some embodiments, disease burden, e.g. tumor burden, is assessed by measuring the number or extent of metastases. In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of disease or condition burden is specified. In some embodiments, exemplary parameters for determination include particular clinical outcomes indicative of amelioration or improvement in the disease or condition, e.g., tumor. Such parameters include: duration of disease control, including complete response (CR), partial response (PR) or stable disease (SD) (see, e.g., Response Evaluation Criteria In Solid Tumors (RECIST) guidelines), objective response rate (ORR), progression-free survival (PFS) and overall survival (OS). Specific thresholds for the parameters can be set to determine the efficacy of the method of combination therapy provided herein.

In some aspects, disease burden is measured or detected prior to administration of the immunotherapy, e.g. T cell therapy, following the administration of the immunotherapy, e.g. T cell therapy but prior to administration of the DGK inhibitor, and/or following the administration of both the immunotherapy, e.g. T cell therapy and the DGK inhibitor. In the context of multiple administration of one or more steps of the combination therapy, disease burden in some embodiments may be measured prior to, or following administration of any of the steps, doses and/or cycles of administration, or at a time between administration of any of the steps, doses and/or cycles of administration.

In some embodiments, the burden is decreased by or by at least at or about 10, 20, 30, 40, 50, 60, 70, 80, 90, or 100 percent by the provided methods compared to immediately prior to the administration of the DGK inhibitor and the immunotherapy, e.g. T cell therapy. In some embodiments, disease burden, tumor size, tumor volume, tumor mass, and/or tumor load or bulk is reduced following administration of the immunotherapy, e.g. T cell therapy and the DGK inhibitor, by at least at or about 10, 20, 30, 40, 50, 60, 70, 80, 90% or more compared to that immediately prior to the administration of the immunotherapy, e.g. T cell therapy and/or the DGK inhibitor.

In some embodiments, reduction of disease burden by the method comprises an induction in morphologic complete remission, for example, as assessed at 1 month, 2 months, 3 months, 4 months, 5 months, 6 months, or more than 6 months, after administration of, e.g., initiation of, the combination therapy.

In some aspects, an assay for minimal residual disease, for example, as measured by multiparametric flow cytometry, is negative, or the level of minimal residual disease is less than about 0.3%, less than about 0.2%, less than about 0.1%, or less than about 0.05%.

In some embodiments, the event-free survival rate or overall survival rate of the subject is improved by the methods, as compared with other methods. For example, in some embodiments, event-free survival rate or probability for subjects treated by the methods at 6 months following the method of combination therapy provided herein, is greater than about 40%, greater than about 50%, greater than about 60%, greater than about 70%, greater than about 80%, greater than about 90%, or greater than about 95%. In some aspects, overall survival rate is greater than about 40%, greater than about 50%, greater than about 60%, greater than about 70%, greater than about 80%, greater than about 90%, or greater than about 95%. In some embodiments, the subject treated with the methods exhibits event-free survival, relapse-free survival, or survival to at least 6 months, or at least 1, 2, 3, 4, 5, 6, 7, 8, 9, or 10 years. In some embodiments, the time to progression is improved, such as a time to progression of greater than at or about 6 months, or at least 1, 2, 3, 4, 5, 6, 7, 8, 9, or 10 years.

In some embodiments, following treatment by the method, the probability of relapse is reduced as compared to other methods. For example, in some embodiments, the probability of relapse at 6 months following the method of combination therapy, is less than about 80%, less than about 70%, less than about 60%, less than about 50%, less than about 40%, less than about 30%, less than about 20%, or less than about 10%.

In some cases, the pharmacokinetics of administered cells, e.g., adoptively transferred cells are determined to assess the availability, e.g., bioavailability of the administered cells. Methods for determining the pharmacokinetics of adoptively transferred cells may include drawing peripheral blood from subjects that have been administered engineered cells, and determining the number or ratio of the engineered cells in the peripheral blood. Approaches for selecting and/or isolating cells may include use of chimeric antigen receptor (CAR)-specific antibodies (e.g., Brentjens et al., Sci. Transl. Med. 2013 March; 5(177): 177ra38) Protein L (Zheng et al., J. Transl. Med. 2012 February; 10:29), epitope tags, such as Strep-Tag sequences, introduced directly into specific sites in the CAR, whereby binding reagents for Strep-Tag are used to directly assess the CAR (Liu et al. (2016) Nature Biotechnology, 34:430; international patent application Pub. No. WO2015095895) and monoclonal antibodies that specifically bind to a CAR polypeptide (see international patent application Pub. No. WO2014190273). Extrinsic marker genes may in some cases be utilized in connection with engineered cell therapies to permit detection or selection of cells and, in some cases, also to promote cell suicide. A truncated epidermal growth factor receptor (EGFRt) in some cases can be co-expressed with a transgene of interest (e.g., a CAR) in transduced cells (see e.g. U.S. Pat. No. 8,802,374). EGFRt may contain an epitope recognized by the antibody cetuximab (Erbitux®) or other therapeutic anti-EGFR antibody or binding molecule, which can be used to identify or select cells that have been engineered with the EGFRt construct and another recombinant receptor, such as a chimeric antigen receptor (CAR), and/or to eliminate or separate cells expressing the receptor. See U.S. Pat. No. 8,802,374 and Liu et al., Nature Biotech. 2016 April; 34(4): 430-434).

In some embodiments, the number of CAR+ T cells in a biological sample obtained from the patient, e.g., blood, can be determined at a period of time after administration of the cell therapy, e.g., to determine the pharmacokinetics of the cells. In some embodiments, number of CAR+ T cells, optionally CAR+CD8+ T cells and/or CAR+CD4+ T cells, detectable in the blood of the subject, or in a majority of subjects so treated by the method, is greater than 1 cells per μL, greater than 5 cells per μL or greater than per 10 cells per μL.

### IV. TOXICITY AND ADVERSE OUTCOMES

In embodiments of the provided methods, the subject is monitored for toxicity or other adverse outcome, including treatment related outcomes, e.g., cytokine release syndrome (CRS) or neurotoxicity (NT), in subjects administered the provided combination therapy comprising a cell therapy (e.g., a T cell therapy) and DGK inhibitor. In some embodiments, the provided methods are carried out to reduce the risk of a toxic outcome or symptom, toxicity-promoting profile, factor, or property, such as a symptom or outcome associated with or indicative of severe cytokine release syndrome (CRS) or severe neurotoxicity.

In some embodiments, the provided methods do not result in a high rate or likelihood of toxicity or toxic outcomes, or reduces the rate or likelihood of toxicity or toxic outcomes, such as severe neurotoxicity (NT) or severe cytokine release syndrome (CRS), such as compared to certain other cell therapies. In some embodiments, the methods do not result in, or do not increase the risk of, severe NT (sNT), severe CRS (sCRS), macrophage activation syndrome, tumor lysis syndrome, fever of at least at or about 38 degrees Celsius for three or more days and a plasma level of CRP of at least at or about 20 mg/dL. In some embodiments, greater than or greater than about 30%, 35%, 40%, 50%, 55%, 60% or more of the subjects treated according to the provided methods do not exhibit any grade of CRS or any grade of neurotoxicity. In some embodiments, no more than 50% of subjects treated (e.g. at least 60%, at least 70%, at least 80%, at least 90% or more of the subjects treated) a cytokine release syndrome (CRS) higher than grade 2 and/or a neurotoxicity higher than grade 2. In some embodiments, at least 50% of subjects treated according to the method (e.g. at least 60%, at least 70%, at least 80%, at least 90% or more of the subjects treated) do not exhibit a severe toxic outcome (e.g. severe CRS or severe neurotoxicity), such as do not exhibit grade 3 or higher neurotoxicity and/or does not exhibit severe CRS, or does not do so within a certain period of time following the treatment, such as within a week, two weeks, or one month of the administration of the cells.

In some aspects, the subject is monitored for and/or the methods reduce the risk for a toxic outcome that is or is associated with or indicative of cytokine release syndrome (CRS) or severe CRS (sCRS). CRS, e.g., sCRS, can occur in some cases following adoptive T cell therapy and administration to subjects of other biological products. See Davila et al., Sci Transl Med 6, 224ra25 (2014); Brentjens et al., Sci. Transl. Med. 5, 177ra38 (2013); Grupp et al., N. Engl. J. Med. 368, 1509-1518 (2013); and Kochenderfer et al., Blood 119, 2709-2720 (2012); Xu et al., Cancer Letters 343 (2014) 172-78.

Typically, CRS is caused by an exaggerated systemic immune response mediated by, for example, T cells, B cells, NK cells, monocytes, and/or macrophages. Such cells may release a large amount of inflammatory mediators such as cytokines and chemokines. Cytokines may trigger an acute inflammatory response and/or induce endothelial organ damage, which may result in microvascular leakage, heart failure, or death. Severe, life-threatening CRS can lead to pulmonary infiltration and lung injury, renal failure, or disseminated intravascular coagulation. Other severe, life-threatening toxicities can include cardiac toxicity, respiratory distress, neurologic toxicity and/or hepatic failure. CRS may be treated using anti-inflammatory therapy such as an anti-IL-6 therapy, e.g., anti-IL-6 antibody, e.g., tocilizumab, or antibiotics or other agents as described.

Outcomes, signs and symptoms of CRS are known and include those described herein. In some embodiments, where a particular dosage regimen or administration effects or does not effect a given CRS-associated outcome, sign, or symptom, particular outcomes, signs, and symptoms and/or quantities or degrees thereof may be specified.

In the context of administering CAR-expressing cells, CRS typically occurs 6-20 days after infusion of cells that express a CAR. See Xu et al., Cancer Letters 343 (2014) 172-78. In some cases, CRS occurs less than 6 days or more than 20 days after CAR T cell infusion. The incidence and timing of CRS may be related to baseline cytokine levels or tumor burden at the time of infusion. Commonly, CRS involves elevated serum levels of interferon (IFN)-γ, tumor necrosis factor (TNF)-α, and/or interleukin (IL)-2. Other cytokines that may be rapidly induced in CRS are IL-1β, IL-6, IL-8, and IL-10.

Exemplary outcomes associated with CRS include fever, rigors, chills, hypotension, dyspnea, acute respiratory distress syndrome (ARDS), encephalopathy, ALT/AST elevation, renal failure, cardiac disorders, hypoxia, neurologic disturbances, and death. Neurological complications include delirium, seizure-like activity, confusion, word-finding difficulty, aphasia, and/or becoming obtunded. Other CRS-related outcomes include fatigue, nausea, headache, seizure, tachycardia, myalgias, rash, acute vascular leak syndrome, liver function impairment, and renal failure. In some aspects, CRS is associated with an increase in one or more factors such as serum-ferritin, d-dimer, aminotransferases, lactate dehydrogenase and triglycerides, or with hypofibrinogenemia or hepatosplenomegaly.

In some embodiments, outcomes associated with CRS include one or more of: persistent fever, e.g., fever of a specified temperature, e.g., greater than at or about 38 degrees Celsius, for two or more, e.g., three or more, e.g., four or more days or for at least three consecutive days; fever greater than at or about 38 degrees Celsius; elevation of cytokines, such as a max fold change, e.g., of at least at or about 75, compared to pre-treatment levels of at least two cytokines (e.g., at least two of the group consisting of interferon gamma (IFNγ), GM-CSF, IL-6, IL-10, Flt-3L, fracktalkine, and IL-5, and/or tumor necrosis factor alpha (TNFα)), or a max fold change, e.g., of at least at or about 250 of at least one of such cytokines; and/or at least one clinical sign of toxicity, such as hypotension (e.g., as measured by at least one intravenous vasoactive pressor); hypoxia (e.g., plasma oxygen (PO₂) levels of less than at or about 90%); and/or one or more neurologic disorders (including mental status changes, obtundation, and seizures).

Exemplary CRS-related outcomes include increased or high serum levels of one or more factors, including cytokines and chemokines and other factors associated with CRS. Exemplary outcomes further include increases in synthesis or secretion of one or more of such factors. Such synthesis or secretion can be by the T cell or a cell that interacts with the T cell, such as an innate immune cell or B cell.

In some embodiments, the CRS-associated serum factors or CRS-related outcomes include inflammatory cytokines and/or chemokines, including interferon gamma (IFN-γ), TNF-a, IL-1β, IL-2, IL-6, IL-7, IL-8, IL-10, IL-12, sIL-2Ra, granulocyte macrophage colony stimulating factor (GM-CSF), macrophage inflammatory protein (MIP)-1, tumor necrosis factor alpha (TNFα), IL-6, and IL-10, IL-1β, IL-8, IL-2, MIP-1, Flt-3L, fracktalkine, and/or IL-5. In some embodiments, the factor or outcome includes C reactive protein (CRP). In addition to being an early and easily measurable risk factor for CRS, CRP also is a marker for cell expansion. In some embodiments, subjects that are measured to have high levels of CRP, such as ≥15 mg/dL, have CRS. In some embodiments, subjects that are measured to have high levels of CRP do not have CRS. In some embodiments, a measure of CRS includes a measure of CRP and another factor indicative of CRS.

In some embodiments, one or more inflammatory cytokines or chemokines are monitored before, during, or after T cell therapy treatment and/or DGK inhibitor treatment. In some aspects, the one or more cytokines or chemokines include IFN-γ, TNF-α, IL-2, IL-1β, IL-6, IL-7, IL-8, IL-10, IL-12, sIL-2Rα, granulocyte macrophage colony stimulating factor (GM-CSF), or macrophage inflammatory protein (MIP). In some embodiments, IFN-γ, TNF-α, and IL-6 are monitored.

CRS criteria that appear to correlate with the onset of CRS to predict which patients are more likely to be at risk for developing sCRS have been developed (see Davilla et al. Science translational medicine. 2014; 6(224): 224ra25). Factors include fevers, hypoxia, hypotension, neurologic changes, elevated serum levels of inflammatory cytokines, such as a set of seven cytokines (IFNγ, IL-5, IL-6, IL-10, Flt-3L, fractalkine, and GM-CSF) whose treatment-induced elevation can correlate well with both pretreatment tumor burden and sCRS symptoms. Other guidelines on the diagnosis and management of CRS are known (see e.g., Lee et al, Blood. 2014; 124(2): 188-95). In some embodiments, the criteria reflective of CRS grade are those detailed in Table 2 below.

TABLE 2
Exemplary Grading Criteria for CRS

| Grade | Description of Symptoms |
|---|---|
| 1 | Not life-threatening, require only symptomatic |
| Mild | treatment such as antipyretics and anti-emetics (e.g., |
| | fever, nausea, fatigue, headache, myalgias, malaise) |
| 2 | Require and respond to moderate intervention: |
| Moderate | Oxygen requirement <40%, or |
| | Hypotension responsive to fluids or low dose of a |
| | single vasopressor, or |
| | Grade 2 organ toxicity (by CTCAE v4.0) |
| 3 | Require and respond to aggressive intervention: |
| Severe | Oxygen requirement ≥40%, or |
| | Hypotension requiring high dose of a single |
| | vasopressor (e.g., norepinephrine ≥20 μg/kg/min, |
| | dopamine ≥10 μg/kg/min, phenylephrine ≥200 |
| | μg/kg/min, or epinephrine ≥10 μg/kg/min), or |
| | Hypotension requiring multiple vasopressors (e.g., |
| | vasopressin + one of the above agents, or combination |
| | vasopressors equivalent to ≥20 μg/kg/min |
| | norepinephrine), or |
| | Grade 3 organ toxicity or Grade 4 transaminitis (by |
| | CTCAE v4.0) |
| 4 | Life-threatening: |
| Life-threatening | Requirement for ventilator support, or |
| | Grade 4 organ toxicity (excluding transaminitis) |
| 5 | Death |
| Fatal | |

In some embodiments, a subject is deemed to develop “severe CRS” (“sCRS”) in response to or secondary to administration of a cell therapy or dose of cells thereof, if, following administration, the subject displays: (1) fever of at least 38 degrees Celsius for at least three days; (2) cytokine elevation that includes either (a) a max fold change of at least 75 for at least two of the following group of seven cytokines compared to the level immediately following the administration: interferon gamma (IFNγ), GM-CSF, IL-6, IL-10, Flt-3L, fracktalkine, and IL-5 and/or (b) a max fold change of at least 250 for at least one of the following group of seven cytokines compared to the level immediately following the administration: interferon gamma (IFNγ), GM-CSF, IL-6, IL-10, Flt-3L, fracktalkine, and IL-5; and (c) at least one clinical sign of toxicity such as hypotension (requiring at least one intravenous vasoactive pressor) or hypoxia (PO₂<90%) or one or more neurologic disorder(s) (including mental status changes, obtundation, and/or seizures). In some embodiments, severe CRS includes CRS with a grade of 3 or greater, such as set forth in Table 2.

In some embodiments, outcomes associated with severe CRS or grade 3 CRS or greater, such as grade 4 or greater, include one or more of: persistent fever, e.g., fever of a specified temperature, e.g., greater than at or about 38 degrees Celsius, for two or more, e.g., three or more, e.g., four or more days or for at least three consecutive days; fever greater than at or about 38 degrees Celsius; elevation of cytokines, such as a max fold change, e.g., of at least at or about 75, compared to pre-treatment levels of at least two cytokines (e.g., at least two of the group consisting of interferon gamma (IFNγ), GM-CSF, IL-6, IL-10, Flt-3L, fracktalkine, and IL-5, and/or tumor necrosis factor alpha (TNFα)), or a max fold change, e.g., of at least at or about 250 of at least one of such cytokines; and/or at least one clinical sign of toxicity, such as hypotension (e.g., as measured by at least one intravenous vasoactive pressor); hypoxia (e.g., plasma oxygen (PO₂) levels of less than at or about 90%); and/or one or more neurologic disorders (including mental status changes, obtundation, and seizures). In some embodiments, severe CRS includes CRS that requires management or care in the intensive care unit (ICU).

In some embodiments, the CRS, such as severe CRS, encompasses a combination of (1) persistent fever (fever of at least 38 degrees Celsius for at least three days) and (2) a serum level of CRP of at least at or about 20 mg/dL. In some embodiments, the CRS encompasses hypotension requiring the use of two or more vasopressors or respiratory failure requiring mechanical ventilation. In some embodiments, the dosage of vasopressors is increased in a second or subsequent administration.

In some embodiments, severe CRS or grade 3 CRS encompasses an increase in alanine aminotransferase, an increase in aspartate aminotransferase, chills, febrile neutropenia, headache, left ventricular dysfunction, encephalopathy, hydrocephalus, and/or tremor.

The method of measuring or detecting the various outcomes may be specified.

In some aspects, the toxic outcome of a therapy, such as a cell therapy, is or is associated with or indicative of neurotoxicity or severe neurotoxicity. In some embodiments, symptoms associated with a clinical risk of neurotoxicity include confusion, delirium, expressive aphasia, obtundation, myoclonus, lethargy, altered mental status, convulsions, seizure-like activity, seizures (optionally as confirmed by electroencephalogram [EEG]), elevated levels of beta amyloid (Aβ), elevated levels of glutamate, and elevated levels of oxygen radicals. In some embodiments, neurotoxicity is graded based on severity (e.g., using a Grade 1-5 scale (see, e.g., Guido Cavaletti & Paola Marmiroli Nature Reviews Neurology 6, 657-666 (December 2010); National Cancer Institute-Common Toxicity Criteria version 4.03 (NCI-CTCAE v4.03).

In some instances, neurologic symptoms may be the earliest symptoms of sCRS. In some embodiments, neurologic symptoms are seen to begin 5 to 7 days after cell therapy infusion. In some embodiments, duration of neurologic changes may range from 3 to 19 days. In some cases, recovery of neurologic changes occurs after other symptoms of sCRS have resolved. In some embodiments, time or degree of resolution of neurologic changes is not hastened by treatment with anti-IL-6 and/or steroid(s).

In some embodiments, a subject is deemed to develop “severe neurotoxicity” in response to or secondary to administration of a cell therapy or dose of cells thereof, if, following administration, the subject displays symptoms that limit self-care (e.g. bathing, dressing and undressing, feeding, using the toilet, taking medications) from among: 1) symptoms of peripheral motor neuropathy, including inflammation or degeneration of the peripheral motor nerves; 2) symptoms of peripheral sensory neuropathy, including inflammation or degeneration of the peripheral sensory nerves, dysesthesia, such as distortion of sensory perception, resulting in an abnormal and unpleasant sensation, neuralgia, such as intense painful sensation along a nerve or a group of nerves, and/or paresthesia, such as functional disturbances of sensory neurons resulting in abnormal cutaneous sensations of tingling, numbness, pressure, cold and warmth in the absence of stimulus. In some embodiments, severe neurotoxicity includes neurotoxicity with a grade of 3 or greater, such as set forth in Table 3. In some embodiments, a severe neurotoxicity is deemed to be a prolonged grade 3 if symptoms or grade 3 neurotoxicity last for 10 days or longer.

TABLE 3
Exemplary Grading Criteria for Neurotoxicity

| Grade | Description of Symptoms |
|---|---|
| 1 | Mild or asymptomatic symptoms |
| Asymptomatic | |
| or Mild | |
| 2 | Presence of symptoms that limit instrumental activities of |
| Moderate | daily living (ADL), such as preparing meals, shopping |
| | for groceries or clothes, using the telephone, managing |
| | money |
| 3 | Presence of symptoms that limit self-care ADL, such as |
| Severe | bathing, dressing and undressing, feeding self, using the |
| | toilet, taking medications |
| 4 | Symptoms that are life-threatening, requiring urgent |
| Life- | intervention |
| threatening | |
| 5 | Death |
| Fatal | |

In some embodiments, the methods reduce symptoms associated with CRS or neurotoxicity compared to other methods. In some aspects, the provided methods reduce symptoms, outcomes or factors associated with CRS, including symptoms, outcomes or factors associated with severe CRS or grade 3 or higher CRS, compared to other methods. For example, subjects treated according to the present methods may lack detectable and/or have reduced symptoms, outcomes or factors of CRS, e.g. severe CRS or grade 3 or higher CRS, such as any described, e.g. set forth in Table 2. In some embodiments, subjects treated according to the present methods may have reduced symptoms of neurotoxicity, such as limb weakness or numbness, loss of memory, vision, and/or intellect, uncontrollable obsessive and/or compulsive behaviors, delusions, headache, cognitive and behavioral problems including loss of motor control, cognitive deterioration, and autonomic nervous system dysfunction, and sexual dysfunction, compared to subjects treated by other methods. In some embodiments, subjects treated according to the present methods may have reduced symptoms associated with peripheral motor neuropathy, peripheral sensory neuropathy, dysethesia, neuralgia or paresthesia.

In some embodiments, the methods reduce outcomes associated with neurotoxicity including damages to the nervous system and/or brain, such as the death of neurons. In some aspects, the methods reduce the level of factors associated with neurotoxicity such as beta amyloid (Aβ), glutamate, and oxygen radicals.

In some embodiments, the toxicity outcome is a dose-limiting toxicity (DLT). In some embodiments, the toxic outcome is the absence of a dose-limiting toxicity. In some embodiments, a dose-limiting toxicity (DLT) is defined as any grade 3 or higher toxicity as described or assessed by any known or published guidelines for assessing the particular toxicity, such as any described above and including the National Cancer Institute (NCI) Common Terminology Criteria for Adverse Events (CTCAE) version 4.0.

In some embodiments, the provided embodiments result in a low rate or risk of developing a toxicity, e.g. CRS or neurotoxicity or severe CRS or neurotoxicity, e.g. grade 3 or higher CRS or neurotoxicity, such as observed with administering a dose of T cells in accord with the provided combination therapy, and/or with the provided articles of manufacture or compositions. In some casesm this permits administration of the cell therapy on an outpatient basis. In some embodiments, the administration of the cell therapy, e.g. dose of T cells (e.g. TCR+ or CAR+ T cells) in accord with the provided methods, and/or with the provided articles of manufacture or compositions, is performed on an outpatient basis or does not require admission to the subject to the hospital, such as admission to the hospital requiring an overnight stay.

In some aspects, subjects administered the cell therapy, e.g. dose of T cells (e.g. TCR+ or CAR+ T cells) in accord with the provided methods, and/or with the provided articles of manufacture or compositions, including subjects treated on an outpatient basis, are not administered an intervention for treating any toxicity prior to or with administration of the cell dose, unless or until the subject exhibits a sign or symptom of a toxicity, such as of a neurotoxicity or CRS.

In some embodiments, if a subject administered the cell therapy, e.g. dose of T cells (e.g. TCR+ or CAR+ T cells), including subjects treated on an outpatient basis, exhibits a fever the subject is given or is instructed to receive or administer a treatment to reduce the fever. In some embodiments, the fever in the subject is characterized as a body temperature of the subject that is (or is measured at) at or above a certain threshold temperature or level. In some aspects, the threshold temperature is that associated with at least a low-grade fever, with at least a moderate fever, and/or with at least a high-grade fever. In some embodiments, the threshold temperature is a particular temperature or range. For example, the threshold temperature may be at or about or at least at or about 38, 39, 40, 41, or 42 degrees Celsius, and/or may be a range of at or about 38 degrees Celsius to at or about 39 degrees Celsius, a range of at or about 39 degrees Celsius to at or about 40 degrees Celsius, a range of at or about 40 degrees Celsius to at or about 41 degrees, or a range of at or about 41 degrees Celsius to at or about 42 degrees Celsius.

In some embodiments, the treatment designed to reduce fever includes treatment with an antipyretic. An antipyretic may include any agent, composition, or ingredient, that reduces fever, such as one of any number of agents known to have antipyretic effects, such as NSAIDs (such as ibuprofen, naproxen, ketoprofen, and nimesulide), salicylates, such as aspirin, choline salicylate, magnesium salicylate, and sodium salicylate, paracetamol, acetaminophen, Metamizole, Nabumetone, Phenaxone, antipyrine, febrifuges. In some embodiments, the antipyretic is acetaminophen. In some embodiments, acetaminophen can be administered at a dose of 12.5 mg/kg orally or intravenously up to every four hours. In some embodiments, it is or comprises ibuprofen or aspirin.

In some embodiments, if the fever is a sustained fever, the subject is administered an alternative treatment for treating the toxicity. For subjects treated on an outpatient basis, the subject is instructed to return to the hospital if the subject has and/or is determined to or to have a sustained fever. In some embodiments, the subject has, and/or is determined to or considered to have, a sustained fever if he or she exhibits a fever at or above the relevant threshold temperature, and where the fever or body temperature of the subject is not reduced, or is not reduced by or by more than a specified amount (e.g., by more than 1 °C, and generally does not fluctuate by about, or by more than about, 0.5 °C, 0.4 °C, 0.3 °C, or 0.2 °C), following a specified treatment, such as a treatment designed to reduce fever such as treatment with an antipyreticm, e.g. NSAID or salicylates, e.g. ibuprofen, acetaminophen or aspirin. For example, a subject is considered to have a sustained fever if he or she exhibits or is determined to exhibit a fever of at least at or about 38 or 39 degrees Celsius, which is not reduced by or is not reduced by more than at or about 0.5 °C, 0.4 °C, 0.3 °C, or 0.2 °C, or by at or about 1%, 2%, 3%, 4%, or 5%, over a period of 6 hours, over a period of 8 hours, or over a period of 12 hours, or over a period of 24 hours, even following treatment with the antipyretic such as acetaminophen. In some embodiments, the dosage of the antipyretic is a dosage ordinarily effective in such as subject to reduce fever or fever of a particular type such as fever associated with a bacterial or viral infection, e.g., a localized or systemic infection.

In some embodiments, the subject has, and/or is determined to or considered to have, a sustained fever if he or she exhibits a fever at or above the relevant threshold temperature, and where the fever or body temperature of the subject does not fluctuate by about, or by more than about, 1 °C, and generally does not fluctuate by about, or by more than about, 0.5 °C, 0.4 °C, 0.3 °C, or 0.2 °C. Such absence of fluctuation above or at a certain amount generally is measured over a given period of time (such as over a 24-hour, 12-hour, 8-hour, 6-hour, 3-hour, or 1-hour period of time, which may be measured from the first sign of fever or the first temperature above the indicated threshold). For example, in some embodiments, a subject is considered to or is determined to exhibit sustained fever if he or she exhibits a fever of at least at or about or at least at or about 38 or 39 degrees Celsius, which does not fluctuate in temperature by more than at or about 0.5 °C, 0.4 °C, 0.3 °C, or 0.2 °C, over a period of 6 hours, over a period of 8 hours, or over a period of 12 hours, or over a period of 24 hours.

In some embodiments, the fever is a sustained fever; in some aspects, the subject is treated at a time at which a subject has been determined to have a sustained fever, such as within one, two, three, four, five six, or fewer hours of such determination or of the first such determination following the initial therapy having the potential to induce the toxicity, such as the cell therapy, such as dose of T cells, e.g. TCR+ or CAR+ T cells.

In some embodiments, one or more interventions or agents for treating the toxicity, such as a toxicity-targeting therapies, is administered at a time at which or immediately after which the subject is determined to or confirmed to (such as is first determined or confirmed to) exhibit sustained fever, for example, as measured according to any of the aforementioned embodiments. In some embodiments, the one or more toxicity-targeting therapies is administered within a certain period of time of such confirmation or determination, such as within 30 minutes, 1 hour, 2 hours, 3 hours, 4 hours, 6 hours, or 8 hours thereof.

### V. ARTICLES OF MANUFACTURE AND KITS

Also provided are articles of manufacture containing an inhibitor of DGKα and/or DGKζ, and components for the T cell therapy, e.g. engineered cells, and/or compositions thereof. The articles of manufacture may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition.

The article of manufacture may include (a) a first container with a composition contained therein, wherein the composition includes the engineered cells used for the T cell therapy (e.g., any of the compositions described in Section I-A); and (b) a second container with a composition contained therein, wherein the composition includes the second agent, such as an inhibitor of DGKα and/or DGKζ (e.g., any of the inhibitors described in Section I-B). The article of manufacture may further include a third container with a composition contained therein, wherein the composition includes a checkpoint inhibitor (e.g., any as described in Section I-C). The article of manufacture may further include a package insert indicating that the compositions can be used to treat a particular condition. Alternatively, or additionally, the article of manufacture may further include another or the same container comprising a pharmaceutically-acceptable buffer. It may further include other materials such as other buffers, diluents, filters, needles, and/or syringes.

### VI. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known in this technical field. Reference to “about” a value or parameter herein includes (and describes) embodiments that are directed to that value or parameter per se. For example, description referring to “about X” includes description of “X”.

As used herein, a “subject” is a human. As used herein, the term “subject” and “patient” are used interchangeably to refer to a human unless specifically stated otherwise.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or engineered cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the immunomodulatory polypeptides or engineered cells administered. In some embodiments, the provided methods involve administering the immunomodulatory polypeptides, engineered cells, or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, one can identify corresponding residues, for example, using conserved and identical amino acid residues as guides. In general, to identify corresponding positions, the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48:1073).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as retroviral, e.g., gammaretroviral and lentiviral vectors.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways, for instance, using publicly available computer software such as BLAST, BLAST-2, ALIGN or Megalign (DNASTAR) software. Appropriate parameters for aligning sequences, including any algorithms needed to achieve maximal alignment over the full length of the sequences being compared, can be determined.

As used herein, a composition refers to any mixture of two or more products, substances, or compounds, including cells. It may be a solution, a suspension, liquid, powder, a paste, aqueous, non-aqueous or any combination thereof.

“Inhibitors of DGKα and/or DGKζ” refers to “inhibitors of DGKα and/or DGKζ enzyme activity,” both of which refer to inhibitors of human DGKα and/or human DGKζ, such as DGKα having the amino acid sequence shown in SEQ ID NO: 190, or the amino acid sequence shown in SEQ ID NO: 190 without the amino acids that are not naturally present in DGKα (e.g., the His tail or certain N-terminal amino acids) and DGKζ having the amino acid sequence shown in SEQ ID NO: 191, or the amino acid sequence shown in SEQ ID NO: 191 without the amino acids that are not naturally present in DGKζ (e.g., the His tail or certain N-terminal amino acids).

A target protein as used herein, e.g., DGK, PD-1, PD-L1 and CTLA4, refers to the human target protein, unless specifically indicated otherwise or the context clearly indicates otherwise. For example, “mouse DGK” refers to the mouse version of DGK, as it specifically indicates it.

As used herein, the phrase “compounds and/or pharmaceutically acceptable salts thereof” refers to at least one compound, at least one salt of the compounds, or a combination thereof. For example, compounds of Formula (I) and/or pharmaceutically acceptable salts thereof includes a compound of Formula (I); two compounds of Formula (I); a pharmaceutically acceptable salt of a compound of Formula (I); a compound of Formula (I) and one or more pharmaceutically acceptable salts of the compound of Formula (I); and two or more pharmaceutically acceptable salts of a compound of Formula (I).

Unless otherwise indicated, any atom with unsatisfied valences is assumed to have hydrogen atoms sufficient to satisfy the valences.

Throughout the specification, groups and substituents thereof may be chosen by one skilled in the field to provide stable moieties and compounds.

In accordance with a convention used in the art,

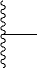

is used in structural formulas herein to depict the bond that is the point of attachment of the moiety or substituent to the core or backbone structure.

The terms “halo” and “halogen,” as used herein, refer to F, Cl, Br, and I.

The term “cyano” refers to the group —CN.

The term “amino” refers to the group —NH₂.

The term “oxo” refers to the group ═O.

The term “alkyl” as used herein, refers to both branched and straight-chain saturated aliphatic hydrocarbon groups containing, for example, from 1 to 12 carbon atoms, from 1 to 6 carbon atoms, and from 1 to 4 carbon atoms. Examples of alkyl groups include, but are not limited to, methyl (Me), ethyl (Et), propyl (e.g., n-propyl and i-propyl), butyl (e.g., n-butyl, i-butyl, sec-butyl, and t-butyl), and pentyl (e.g., n-pentyl, isopentyl, neopentyl), n-hexyl, 2-methylpentyl, 2-ethylbutyl, 3-methylpentyl, and 4-methylpentyl. When numbers appear in a subscript after the symbol “C”, the subscript defines with more specificity the number of carbon atoms that a particular group may contain. For example, “C₁₋₄ alkyl” denotes straight and branched chain alkyl groups with one to four carbon atoms.

The term “fluoroalkyl” as used herein is intended to include both branched and straight-chain saturated aliphatic hydrocarbon groups substituted with one or more fluorine atoms. For example, “C₁₋₄ fluoroalkyl” is intended to include C₁, C₂, C₃, and C₄ alkyl groups substituted with one or more fluorine atoms. Representative examples of fluoroalkyl groups include, but are not limited to, —CF₃ and —CH₂CF₃.

The term “cyanoalkyl” includes both branched and straight-chain saturated alkyl groups substituted with one or more cyano groups. For example, “cyanoalkyl” includes —CH₂CN, —CH₂CH₂CN, and C₁₋₄ cyanoalkyl.

The term “aminoalkyl” includes both branched and straight-chain saturated alkyl groups substituted with one or more amine groups. For example, “aminoalkyl” includes —CH₂NH₂, —CH₂CH₂NH₂, and C₁₋₄ aminoalkyl.

The term “hydroxyalkyl” includes both branched and straight-chain saturated alkyl groups substituted with one or more hydroxyl groups. For example, “hydroxyalkyl” includes —CH₂OH, —CH₂CH₂OH, and C₁₋₄ hydroxyalkyl.

The term “alkenyl” refers to a straight or branched chain hydrocarbon radical containing from 2 to 12 carbon atoms and at least one carbon-carbon double bond. Exemplary such groups include ethenyl or allyl. For example, “C₂₋₆ alkenyl” denotes straight and branched chain alkenyl groups with two to six carbon atoms.

The term “alkynyl” refers to a straight or branched chain hydrocarbon radical containing from 2 to 12 carbon atoms and at least one carbon to carbon triple bond. Exemplary such groups include ethynyl. For example, “C₂₋₆ alkynyl” denotes straight and branched chain alkynyl groups with two to six carbon atoms.

The term “cycloalkyl,” as used herein, refers to a group derived from a non-aromatic monocyclic or polycyclic hydrocarbon molecule by removal of one hydrogen atom from a saturated ring carbon atom. Representative examples of cycloalkyl groups include, but are not limited to, cyclopropyl, cyclopentyl, and cyclohexyl. When numbers appear in a subscript after the symbol “C”, the subscript defines with more specificity the number of carbon atoms that a particular cycloalkyl group may contain. For example, “C₃₋₆ cycloalkyl” denotes cycloalkyl groups with three to six carbon atoms.

The term “alkoxy,” as used herein, refers to an alkyl group attached to the parent molecular moiety through an oxygen atom, for example, methoxy group (—OCH₃). For example, “C₁₋₃ alkoxy” denotes alkoxy groups with one to three carbon atoms.

The terms “fluoroalkoxy” and “—O(fluoroalkyl)” represent a fluoroalkyl group as defined above attached through an oxygen linkage (—O—). For example, “C₁₋₄ fluoroalkoxy” is intended to include C₁, C₂, C₃, and C₄ fluoroalkoxy groups.

The term “alkalenyl” refers to a saturated carbon chain with two attachment points to the core or backbone structure. The alkalenyl group has the structure —(CH₂)ₙ— in which n is an integer of 1 or greater. Examples of alkalenyl linkages include —CH₂CH₂—, —CH₂CH₂CH₂—, and —(CH₂)₂₋₄—.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Compounds, e.g., the compounds of Formula (I) or Formula (II), can form pharmaceutically acceptable salts which can be used in the methods described herein. Unless otherwise indicated, reference to a compound is understood to include reference to one or more pharmaceutically acceptable salts thereof. The term “salt(s)” denotes acidic and/or basic pharmaceutically acceptable salts formed with inorganic and/or organic acids and bases. In addition, the term “salt(s) may include zwitterions (inner salts), e.g., when a compound of Formula (I) contains both a basic moiety, such as an amine or a pyridine or imidazole ring, and an acidic moiety, such as a carboxylic acid. Pharmaceutically acceptable (i.e., non-toxic, physiologically acceptable) salts are preferred, such as, for example, acceptable metal and amine salts in which the cation does not contribute significantly to the toxicity or biological activity of the salt. However, other salts may be useful, e.g., in isolation or purification steps which may be employed during preparation, and thus, are contemplated herein. Salts of compounds, e.g., the compounds of the formula (I), may be formed, for example, by reacting a compound, e.g., a compound of the Formula (I), with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, maleates (formed with maleic acid), 2-hydroxyethanesulfonates, lactates, methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts include ammonium salts, alkali metal salts such as sodium, lithium, and potassium salts; alkaline earth metal salts such as calcium and magnesium salts; barium, zinc, and aluminum salts; salts with organic bases (for example, organic amines) such as trialkylamines such as triethylamine, procaine, dibenzylamine, N-benzyl-β-phenethylamine, 1-ephenamine, N,N′-dibenzylethylene-diamine, dehydroabietylamine, N-ethylpiperidine, benzylamine, dicyclohexylamine or similar pharmaceutically acceptable amines and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others. Preferred salts include monohydrochloride, hydrogensulfate, methanesulfonate, phosphate or nitrate salts.

Compounds, e.g., the compounds of Formula (I) can be provided as amorphous solids or crystalline solids. Lyophilization can be employed to provide the compounds, e.g., the compounds of Formula (I), as a solid.

It should further be understood that solvates (e.g., hydrates) of compounds, e.g., the compounds of Formula (I), can also be used in the methods described herein. The term “solvate” means a physical association of a compound, e.g., a compound of Formula (I), with one or more solvent molecules, whether organic or inorganic. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, isopropanolates, acetonitrile solvates, and ethyl acetate solvates. Methods of solvation are known in the art.

Various forms of prodrugs are well known in the art and are described in:
a) The Practice of Medicinal Chemistry, Camille G. Wermuth et al., Ch 31, (Academic Press, 1996);
b) Design of Prodrugs, edited by H. Bundgaard, (Elsevier, 1985);
c) A Textbook of Drug Design and Development, P. Krogsgaard-Larson and H. Bundgaard, eds. Ch 5, pgs 113-191 (Harwood Academic Publishers, 1991); and
d) Hydrolysis in Drug and Prodrug Metabolism, Bernard Testa and Joachim M. Mayer, (Wiley-VCH, 2003).

In addition, compounds, e.g., compounds of Formula (I), subsequent to their preparation, can be isolated and purified to obtain a composition containing an amount by weight equal to or greater than 99% of a compound, e.g., a compound of Formula (I), (“substantially pure”), which is then used or formulated as described herein. Such “substantially pure” compounds, e.g., compounds of Formula (I), are also contemplated herein.

“Stable compound” and “stable structure” are meant to indicate a compound that the compound is sufficiently robust to survive isolation to a useful degree of purity from a reaction mixture, and formulation into an efficacious therapeutic agent. The compounds for use herein are intended to embody stable compounds.

The compounds described herein are intended to include all isotopes of atoms occurring in the present compounds. Isotopes include those atoms having the same atomic number but different mass numbers. By way of general example and without limitation, isotopes of hydrogen include deuterium (D) and tritium (T). Isotopes of carbon include ¹³C and ¹⁴C. Isotopically-labeled compounds can generally be prepared by conventional techniques known to those skilled in the art or by processes analogous to those described herein, using an appropriate isotopically-labeled reagent in place of the non-labeled reagent otherwise employed.

### VII. EXEMPLARY EMBODIMENTS

Among the provided embodiments are:
1 A method of treatment, the method comprising administering to a subject having a cancer a combination therapy comprising:
(a) a T cell therapy comprising viable engineered T cells expressing a recombinant receptor directed against an antigen expressed by cells of the cancer, wherein the T cell therapy is administered on Day 1 of the combination therapy; and
(b) an inhibitor of DGKα and/or DGKζ, wherein the administration of the inhibitor is initiated on any of Days 1-8, inclusive, of the combination therapy, and the inhibitor is administered once daily for between or between about 21 and 42 consecutive days, inclusive.
2 The method of embodiment 1, wherein the administration of the inhibitor is initiated subsequent to the administration of the T cell therapy.
3 A method of treatment, the method comprising administering to a subject having a cancer an inhibitor of DGKα and/or DGKζ, wherein the inhibitor is administered as part of a combination therapy comprising the inhibitor and a T cell therapy comprising viable engineered T cells expressing a recombinant receptor directed against an antigen expressed by cells of the cancer, wherein:
prior to initiation of administration of the inhibitor, the subject has been previously administered the T cell therapy, wherein the T cell therapy is administered on Day 1 of the combination therapy;
the administration of the inhibitor is initiated on any of Days 1-8, inclusive, of the combination therapy; and
the inhibitor is administered once daily for between or between about 21 and 42 consecutive days, inclusive.
4. A method of treatment, the method comprising administering to a subject having a cancer a combination therapy comprising:
(a) a T cell therapy comprising viable engineered T cells expressing a recombinant receptor directed against an antigen expressed by cells of the cancer, wherein the T cell therapy is administered on Day 1 of the combination therapy; and
(b) an inhibitor of DGKα and/or DGKζ, wherein the administration of the inhibitor is initiated on any of Days 1-8, inclusive, of the combination therapy, and the inhibitor is administered continuously at least until the number of engineered T cells of the T cell therapy has peaked in the subject following administration of the T cell therapy.
5 The method of embodiment 4, wherein the administration of the inhibitor is initiated subsequent to the administration of the T cell therapy.
6 A method of treatment, the method comprising administering to a subject having a cancer an inhibitor of DGKα and/or DGKζ, wherein the inhibitor is administered as part of a combination therapy comprising the inhibitor and a T cell therapy comprising viable engineered T cells expressing a recombinant receptor directed against an antigen expressed by cells of the cancer, wherein:
prior to initiation of administration of the inhibitor, the subject has been previously administered the T cell therapy, wherein the T cell therapy is administered on Day 1 of the combination therapy;
the administration of the inhibitor is initiated on any of Days 1-8, inclusive, of the combination therapy; and
the inhibitor is administered continuously at least until the number of engineered T cells of the T cell therapy has peaked in the subject following administration of the T cell therapy.
7. The method of embodiment 4, wherein the administration of the inhibitor is initiated on Day 1 of the combination therapy prior to the administration of the T cell therapy.
8. A method of treatment, the method comprising administering to a subject having a cancer a T cell therapy comprising viable engineered T cells expressing a recombinant receptor directed against an antigen expressed by cells of the cancer, wherein the T cell therapy is administered as part of a combination therapy comprising the T cell therapy and an inhibitor of DGKα and/or DGKζ, wherein:
the T cell therapy is administered on Day 1 of the combination therapy;
the administration of the inhibitor is initiated on Day 1 of the combination therapy prior to the administration of the T cell therapy; and
the inhibitor is administered continuously at least until the number of engineered T cells of the T cell therapy has peaked in the subject following administration of the T cell therapy.
9. The method of any one of embodiments 4-8, wherein the inhibitor is administered daily, every two days, or every three days.
10. The method of any one of embodiments 4-9, wherein the inhibitor is administered daily.
11. The method of any one of embodiments 4-10, wherein the inhibitor is administered once on each of the days that the inhibitor is administered.
12. The method of any one of embodiments 4-11, wherein the inhibitor is administered until the number of engineered T cells of the T cell therapy has peaked in the subject following administration of the T cell therapy.
13. The method of any one of embodiments 4-12, wherein the inhibitor is administered over a period of time between or between about 21 and 42 days, inclusive.
14. A method of treatment, the method comprising administering to a subject having a cancer a combination therapy comprising:
(a) a T cell therapy comprising viable engineered T cells expressing a recombinant receptor directed against an antigen expressed by cells of the cancer, wherein the T cell therapy is administered on Day 1 of the combination therapy; and
(b) an inhibitor of DGKα and/or DGKζ, wherein the administration of the inhibitor is initiated on any of Days 1-8, inclusive, of the combination therapy, and the inhibitor is administered in a therapeutically effective dose for a period between or between about 21 and 42 days.
15. The method of embodiment 14, wherein the administration of the inhibitor is initiated subsequent to the administration of the T cell therapy.
16. A method of treatment, the method comprising administering to a subject having a cancer an inhibitor of DGKα and/or DGKζ, wherein the inhibitor is administered as part of a combination therapy comprising the inhibitor and a T cell therapy comprising viable engineered T cells expressing a recombinant receptor directed against an antigen expressed by cells of the cancer, wherein:
prior to initiation of administration of the inhibitor, the subject has been previously administered the T cell therapy, wherein the T cell therapy is administered on Day 1 of the combination therapy;
the administration of the inhibitor is initiated on any of Days 1-8, inclusive, of the combination therapy; and
the inhibitor is administered in a therapeutically effective dose for a period between or between about 21 and 42 days.
17. The method of embodiment 14, wherein the administration of the inhibitor is initiated on Day 1 of the combination therapy prior to the administration of the T cell therapy.
18. A method of treatment, the method comprising administering to a subject having a cancer a T cell therapy comprising viable engineered T cells expressing a recombinant receptor directed against an antigen expressed by cells of the cancer, wherein the T cell therapy is administered as part of a combination therapy comprising the T cell therapy and an inhibitor of DGKα and/or DGKζ, wherein:
the T cell therapy is administered on Day 1 of the combination therapy;
the administration of the inhibitor is initiated on Day 1 of the combination therapy prior to the administration of the T cell therapy; and
the inhibitor is administered in a therapeutically effective dose for a period between or between about 21 and 42 days.
19. The method of any one of embodiments 14-18, wherein the therapeutically effective dose provides a dose of the inhibitor that is continuously therapeutically effective for the period between or between about 21 and 42 days.
20. The method of any one of embodiments 14-19, wherein the inhibitor is administered every 2 days or 3 days.
21. The method of any one of embodiments 1-20, wherein the administration of the inhibitor is initiated on any of Days 1-6, inclusive, of the combination therapy.
22. The method of any one of embodiments 1-21, wherein the administration of the inhibitor is initiated on any of Days 1-4, inclusive, of the combination therapy.
23. The method of any one of embodiments 1-22, wherein the administration of the inhibitor is initiated on Day 1 or Day 2 of the combination therapy.
24. The method of any one of embodiments 1-23, wherein the administration of the inhibitor is initiated on Day 1 of the combination therapy.
25. The method of any one of embodiments 1 and 21-24, wherein the administration of the inhibitor is initiated on Day 1 of the combination therapy prior to the administration of the T cell therapy.
26. A method of treatment, the method comprising administering to a subject having a cancer a T cell therapy comprising viable engineered T cells expressing a recombinant receptor directed against an antigen expressed by cells of the cancer, wherein the T cell therapy is administered as part of a combination therapy comprising the T cell therapy and an inhibitor of DGKα and/or DGKζ, wherein:
the T cell therapy is administered on Day 1 of the combination therapy;
the administration of the inhibitor is initiated on Day 1 of the combination therapy prior to the administration of the T cell therapy; and
the inhibitor is administered once daily for between or between about 21 and 42 consecutive days, inclusive.
27. The method of any one of embodiments 1-26, wherein the inhibitor is administered over a period of time between or between about 21 and 35 days, inclusive.
28. The method of any one of embodiments 1-27, wherein the inhibitor is administered over a period of time between or between about 21 and 28 days, inclusive.
29. The method of any one of embodiments 1-28, wherein the inhibitor is administered over a period of time between or between about 28 and 42 days, inclusive.
30. The method of any one of embodiments 1-27 and 29, wherein the inhibitor is administered over a period of time between or between about 28 and 35 days, inclusive.
31. The method of any one of embodiments 1-30, wherein the inhibitor is administered over a period of time that is or is about 28 days.
32. A method of treatment, the method comprising administering to a subject having a cancer a combination therapy comprising:
(a) a T cell therapy comprising viable engineered T cells expressing a recombinant receptor directed against an antigen expressed by cells of the cancer, wherein the T cell therapy is administered on Day 1 of the combination therapy; and
(b) an inhibitor of DGKα and/or DGKζ, wherein the administration of the inhibitor is initiated on Day 1 of the combination therapy, and the inhibitor is administered once daily on Days 1-28, inclusive, of the combination therapy.
33. The method of embodiment 32, wherein the administration of the inhibitor is initiated subsequent to the administration of the T cell therapy.
34. A method of treatment, the method comprising administering to a subject having a cancer an inhibitor of DGKα and/or DGKζ, wherein the inhibitor is administered as part of a combination therapy comprising the inhibitor and a T cell therapy comprising viable engineered T cells expressing a recombinant receptor directed against an antigen expressed by cells of the cancer, wherein:
prior to initiation of administration of the inhibitor, the subject has been previously administered the T cell therapy, wherein the T cell therapy is administered on Day 1 of the combination therapy;
the administration of the inhibitor is initiated on Day 1 of the combination therapy; and
the inhibitor is administered once daily on Days 1-28, inclusive, of the combination therapy.
35. The method of embodiment 32, wherein the administration of the inhibitor is initiated prior to the administration of the T cell therapy.
36. A method of treatment, the method comprising administering to a subject having a cancer a T cell therapy comprising viable engineered T cells expressing a recombinant receptor directed against an antigen expressed by cells of the cancer, wherein the T cell therapy is administered as part of a combination therapy comprising the T cell therapy and an inhibitor of DGKα and/or DGKζ, wherein:
the T cell therapy is administered on Day 1 of the combination therapy;
the administration of the inhibitor is initiated on Day 1 of the combination therapy prior to the administration of the T cell therapy; and
the inhibitor is administered once daily on Days 1-28, inclusive, of the combination therapy.
37. The method of any one of embodiments 1-36, wherein the administration of the inhibitor is initiated within or within about 12 hours of the administration of the T cell therapy.
38. The method of any one of embodiments 1-37, wherein the administration of the inhibitor is initiated within or within about 6 hours of the administration of the T cell therapy.
39. The method of any one of embodiments 1-38, wherein the administration of the inhibitor is initiated within or within about 4 hours of the administration of the T cell therapy.
40. The method of any one of embodiments 1-39, wherein the administration of the inhibitor is initiated within or within about 2 hours of the administration of the T cell therapy.
41. The method of any one of embodiments 1-40, wherein the administration of the inhibitor is initiated within or within about 1 hour of the administration of the T cell therapy.
42. The method of any one of embodiments 1, 4, 9-14, 19-24, 27-32, and 37-41, wherein the administration of the inhibitor is initiated on Day 1 of the combination therapy concurrently with the administration of the T cell therapy.
43. The method of any one of embodiments 1, 4, 9-14, 19-24, 27-32, and 37-42, wherein the administration of the inhibitor is initiated on Day 1 of the combination therapy during the administration of the T cell therapy.
44. The method of any one of embodiments 1-43, wherein prior to the administration of the T cell therapy, the subject has been preconditioned with a lymphodepleting therapy comprising administration of fludarabine and/or cyclophosphamide.
45. The method of embodiment 44, wherein the method further comprises administering the lymphodepleting therapy to the subject.
46. The method of embodiment 44 or embodiment 45, wherein the lymphodepleting therapy comprises administration of:
cyclophosphamide at between or between about 200 and 400 mg/m², inclusive, daily for between or between about 2 and 4 days, inclusive; and/or
fludarabine at between or between about 20 and 40 mg/m², inclusive, daily for between or between about 2 and 4 days, inclusive.
47. The method of any one of embodiments 44-46, wherein the lymphodepleting therapy comprises administration of cyclophosphamide at or at about 300 mg/m² and fludarabine at or at about 30 mg/m² each daily for or for about 3 days.
48. The method of any one of embodiments 44-47, wherein the T cell therapy is administered between or between about 2 and 7 days, inclusive, after the administration of the lymphodepleting therapy.
49. The method of any one of embodiments 1-48, wherein the inhibitor is an inhibitor of DGKα and not a significant inhibitor of DGKζ.
50. The method of any one of embodiments 1-48, wherein the inhibitor is an inhibitor of DGKζ and not a significant inhibitor of DGKα.
51. The method of any one of embodiments 1-48, wherein the inhibitor is an inhibitor of DGKα and DGKζ.
52. The method of any one of embodiments 1-51, wherein the inhibitor is not a significant inhibitor of other DGKs.
53. The method of any one of embodiments 1-52, wherein the inhibitor is a compound of Formula (I):

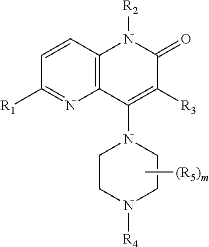

or a pharmaceutically acceptable salt thereof, wherein:
R₁ is H, F, Cl, Br, —CN, C₁₋₃ alkyl substituted with zero to 4 R₁ₐ, C₃₋₄ cycloalkyl substituted with zero to 4 R₁ₐ, C₁₋₃ alkoxy substituted with zero to 4 R₁ₐ, —NRₐRₐ, —S(O)ₙRₑ, or —P(O)RₑRₑ;
each R₁ₐ is independently F, Cl, —CN, —OH, —OCH₃, or —NRₐRₐ;
each Rₐ is independently H or C₁₋₃ alkyl;
each Rₑ is independently C₃₋₄ cycloalkyl or C₁₋₃ alkyl substituted with zero to 4 R₁ₐ;
R₂ is H, C₁₋₃ alkyl substituted with zero to 4 R₂ₐ, or C₃₋₄ cycloalkyl substituted with zero to 4 R₂ₐ;
each R₂ₐ is independently F, Cl, —CN, —OH, —O(C₁₋₂ alkyl), C₃₋₄ cycloalkyl, C₃₋₄ alkenyl, or
C₃₋₄ alkynyl;
R₃ is H, F, Cl, Br, —CN, C₁₋₃ alkyl, C₁₋₂ fluoroalkyl, C₃₋₄ cycloalkyl, C₃₋₄ fluorocycloalkyl, or —NO₂;
R₄ is —CH₂R₄ₐ, —CH₂CH₂R₄ₐ, —CH₂CHR₄ₐR_{4d}, —CHR₄ₐR_{4b}, or —CR₄ₐR_{4b}R_{4c};
R₄ₐ and R_{4b} are independently:
(i) C₁₋₆ alkyl substituted with zero to 4 substituents independently selected from F, Cl, —CN, —OH, —OCH₃, —SCH₃, C₁₋₃ fluoroalkoxy, —NRₐRₐ, —S(O)₂Rₑ, or —NRₐS(O)₂Rₑ;
(ii) C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, C₁₋₄ hydroxyalkyl, —(CH₂)₁₋₂O(C₁₋₃ alkyl), C₁₋₄ alkoxy, —O(C₁₋₄ hydroxyalkyl), —O(CH)₁₋₃O(C₁₋₃ alkyl), C₁₋₃ fluoroalkoxy, —O(CH)₁₋₃NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₃ alkyl)₂, —S(O)₂(C₁₋₃ alkyl), —O(CH₂)₁₋₂(C₃₋₆ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or
(iii) C₁₋₄ alkyl substituted with one cyclic group selected from C₃₋₆ cycloalkyl, heterocyclyl, aryl, and heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl;
or R₄ₐ and R_{4b} together with the carbon atom to which they are attached form a C₃₋₆ cycloalkyl or a 3- to 6-membered heterocyclyl, each substituted with zero to 3 R_{f};
each R_{f} is independently F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, and —NR_{c}R_{c};
R_{4c} is C₁₋₆ alkyl or C₃₋₆ cycloalkyl, each substituted with zero to 4 substituents independently selected from F, Cl, —OH, C₁₋₂ alkoxy, C₁₋₂ fluoroalkoxy, and —CN;
R₄ₐ is —OCH₃;
each R_{c} is independently H or C₁₋₂ alkyl;
R_{d} is phenyl substituted with zero to 1 substituent selected from F, Cl, —CN, —CH₃, and —OCH₃; each R₅ is independently —CN, C₁₋₆ alkyl substituted with zero to 4 R_{g}, C₂₋₄ alkenyl substituted with zero to 4 R_{g}, C₂₋₄ alkynyl substituted with zero to 4 R_{g}, C₃₋₄ cycloalkyl substituted with zero to 4 R_{g}, phenyl substituted with zero to 4 R_{g}, oxadiazolyl substituted with zero to 3 R_{g}, pyridinyl substituted with zero to 4 R_{g}, —(CH₂)₁₋₂(heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each R_{g} is independently F, Cl, —CN, —OH, C₁₋₃ alkoxy, C₁₋₃ fluoroalkoxy, —O(CH₂)₁₋₂O(C₁₋₂ alkyl), or —NR_{c}R_{c};
m is zero, 1, 2, or 3; and
n is zero, 1, or 2.
54. The method of embodiment 53, wherein the inhibitor is a compound of Formula (I) or a pharmaceutically acceptable salt thereof, wherein:
R₁ is H, F, Cl, Br, —CN, C₁₋₃ alkyl substituted with zero to 4 R₁ₐ, cyclopropyl substituted with zero to 3 R₁ₐ, C₁₋₃ alkoxy substituted with zero to 3 R₁ₐ, —NRₐRₐ, —S(O)ₙCH₃, or —P(O)(CH₃)₂;
each R₁ₐ is independently F, Cl, or —CN;
each Rₐ is independently H or C₁₋₃ alkyl;
R₂ is H or C₁₋₂ alkyl substituted with zero to 2 R₂ₐ;
each R₂ₐ is independently F, Cl, —CN, —OH, —O(C₁₋₂ alkyl), cyclopropyl, C₃₋₄ alkenyl, or C₃₋₄ alkynyl;
R₃ is H, F, Cl, Br, —CN, C₁₋₂ alkyl, —CF₃, cyclopropyl, or —NO₂;
R₄ₐ and R_{4b} are independently:
(i) C₁₋₄ alkyl substituted with zero to 4 substituents independently selected from F, Cl, —CN, —OH, —OCH₃, —SCH₃, C₁₋₃ fluoroalkoxy, and —NRₐRₐ;
(ii) C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, —CH₂OH, —(CH₂)₁₋₂O(C₁₋₂ alkyl), C₁₋₄ alkoxy, —O(C₁₋₄ hydroxyalkyl), —O(CH)₁₋₂O(C₁₋₂ alkyl), C₁₋₃ fluoroalkoxy, —O(CH)₁₋₂NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —O(CH₂)₁₋₂(C₃₋₄ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or
(iii) C₁₋₃ alkyl substituted with one cyclic group selected from C₃₋₆ cycloalkyl, heterocyclyl, phenyl, and heteroaryl, said cyclic group substituted with zero to 3 substituents independently selected from F, Cl, Br, —OH, —CN, C₁₋₃ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₄ cycloalkyl;
or R₄ₐ and R_{4b} together with the carbon atom to which they are attached, form a C₃₋₆ cycloalkyl or a 3- to 6-membered heterocyclyl, each substituted with zero to 3 R_{f};
each R_{f} is independently F, Cl, Br, —OH, —CN, C₁₋₄ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₄ alkyl, C₁₋₂ fluoroalkyl, C₁₋₃ alkoxy, C₁₋₂ fluoroalkoxy, and —NR_{c}R_{c};
R_{4c} is C₁₋₄ alkyl or C₃₋₆ cycloalkyl, each substituted with zero to 4 substituents independently selected from F, Cl, —OH, C₁₋₂ alkoxy, C₁₋₂ fluoroalkoxy, and —CN;
and each R₅ is independently —CN, C₁₋₅ alkyl substituted with zero to 4 R_{g}, C₂₋₃ alkenyl substituted with zero to 4 R_{g}, C₂₋₃ alkynyl substituted with zero to 4 R_{g}, C₃₋₄ cycloalkyl substituted with zero to 4 R_{g}, phenyl substituted with zero to 3 R_{g}, oxadiazolyl substituted with zero to 3 R_{g}, pyridinyl substituted with zero to 3 R_{g}, —(CH₂)₁₋₂(heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl),
(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl).
55. The method of embodiment 54, wherein the inhibitor is a compound of Formula (I) or a pharmaceutically acceptable salt thereof having the structure:

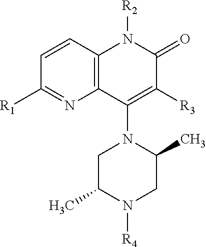

wherein:
R₁ is —CN;
R₂ is —CH₃;
R₃ is H, F, or —CN;
R₄ is:

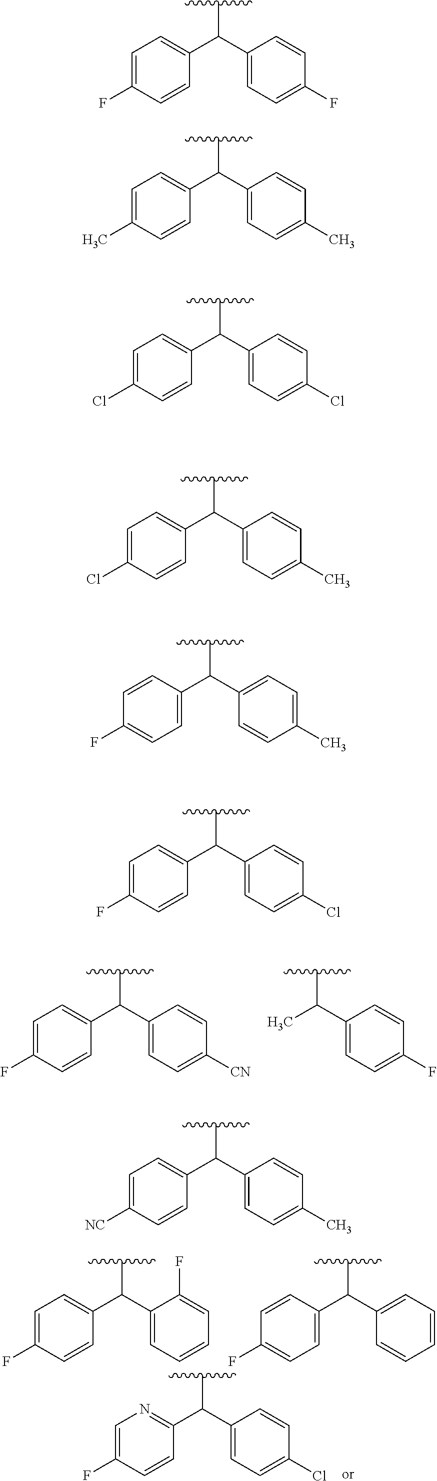

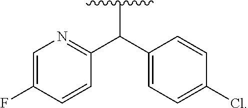 [SMILES: *C(c1ccc(Cl)cc1)c1ccc(F)cn1]

56. The method of embodiment 53, wherein the inhibitor is a compound of Formula (I) or a pharmaceutically acceptable salt thereof having the structure:

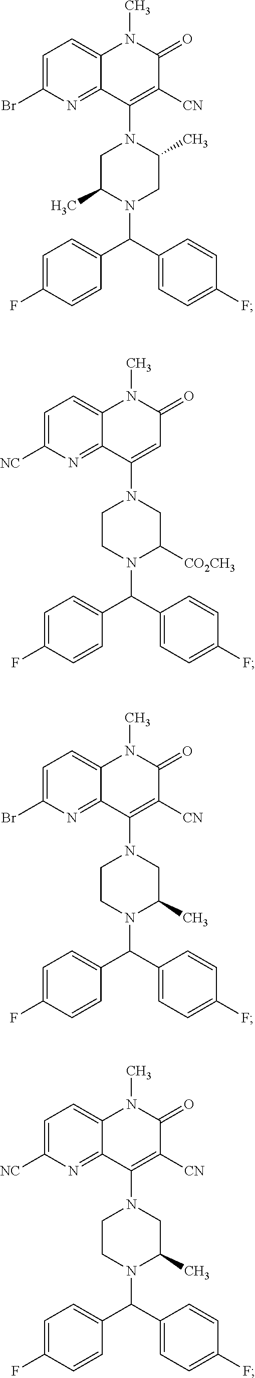 [SMILES: C[C@@H]1CN(C(c2ccc(F)cc2)c2ccc(F)cc2)[C@@H](C)CN1c1c(C#N)c(=O)n(C)c2ccc(Br)nc12 ; COC(=O)C1CN(c2cc(=O)n(C)c3ccc(C#N)nc23)CCN1C(c1ccc(F)cc1)c1ccc(F)cc1 ; C[C@@H]1CN(c2c(C#N)c(=O)n(C)c3ccc(Br)nc23)CCN1C(c1ccc(F)cc1)c1ccc(F)cc1 ; C[C@@H]1CN(c2c(C#N)c(=O)n(C)c3ccc(C#N)nc23)CCN1C(c1ccc(F)cc1)c1ccc(F)cc1]

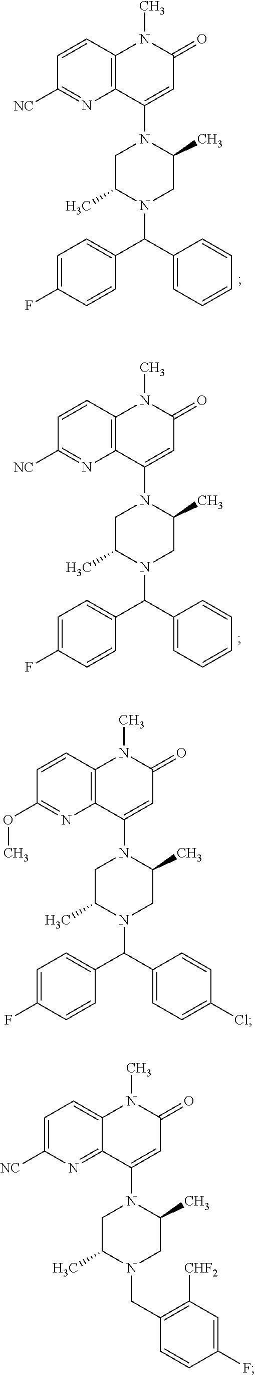 [SMILES: C[C@@H]1CN(c2cc(=O)n(C)c3ccc(C#N)nc23)[C@@H](C)CN1C(c1ccccc1)c1ccc(F)cc1 ; C[C@@H]1CN(c2cc(=O)n(C)c3ccc(C#N)nc23)[C@@H](C)CN1C(c1ccccc1)c1ccc(F)cc1 ; COc1ccc2c(n1)c(N1C[C@@H](C)N(C(c3ccc(F)cc3)c3ccc(Cl)cc3)C[C@@H]1C)cc(=O)n2C ; C[C@@H]1CN(c2cc(=O)n(C)c3ccc(C#N)nc23)[C@@H](C)CN1Cc1ccc(F)cc1C(F)F]

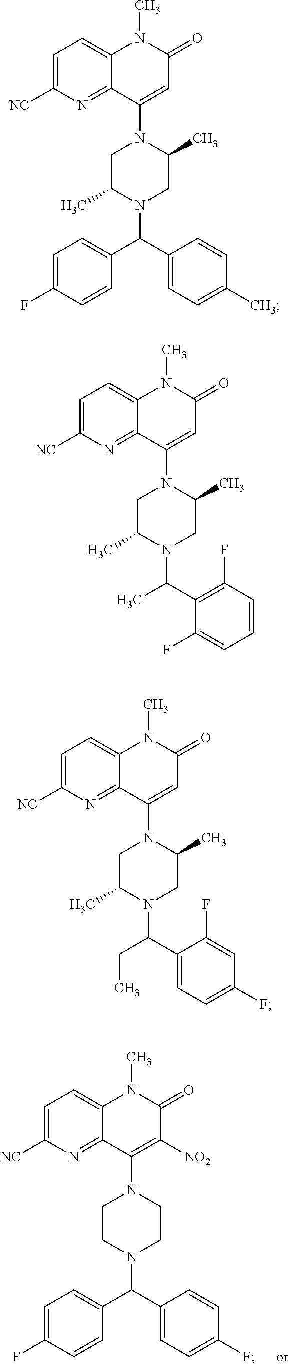 [SMILES: Cc1ccc(C(c2ccc(F)cc2)N2C[C@H](C)N(c3cc(=O)n(C)c4ccc(C#N)nc34)C[C@H]2C)cc1 ; CC(c1c(F)cccc1F)N1C[C@H](C)N(c2cc(=O)n(C)c3ccc(C#N)nc23)C[C@H]1C ; CCC(c1ccc(F)cc1F)N1C[C@H](C)N(c2cc(=O)n(C)c3ccc(C#N)nc23)C[C@H]1C ; Cn1c(=O)c([N+](=O)[O-])c(N2CCN(C(c3ccc(F)cc3)c3ccc(F)cc3)CC2)c2nc(C#N)ccc21]

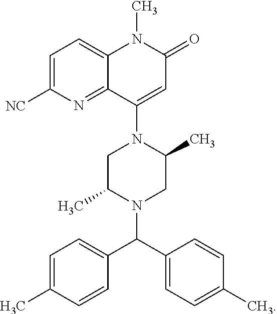 [SMILES: Cc1ccc(C(c2ccc(C)cc2)N2C[C@H](C)N(c3cc(=O)n(C)c4ccc(C#N)nc34)C[C@H]2C)cc1]

57. The method of any one of embodiments 1-52, wherein the inhibitor is a compound of Formula (II):

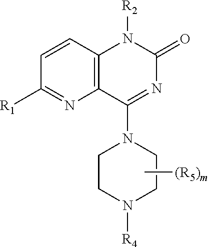

or a salt thereof, wherein:
R₁ is H, F, Cl, Br, —CN, —OH, C₁₋₃ alkyl substituted with zero to 4 R₁ₐ, C₃₋₄ cycloalkyl substituted with zero to 4 R₁ₐ, C₁₋₃ alkoxy substituted with zero to 4 R₁ₐ, —NRₐRₐ, —S(O)ₙRₑ, or —P(O)RₑRₑ;
each R₁ₐ is independently F, Cl, —CN, —OH, —OCH₃, or —NRₐRₐ;
each Rₐ is independently H or C₁₋₃ alkyl;
each Rₑ is independently C₃₋₄ cycloalkyl or C₁₋₃ alkyl substituted with zero to 4 R₁ₐ;
R₂ is H, C₁₋₃ alkyl substituted with zero to 4 R₂ₐ, or C₃₋₄ cycloalkyl substituted with zero to 4 R₂ₐ;
each R₂ₐ is independently F, Cl, —CN, —OH, —O(C₁₋₂ alkyl), C₃₋₄ cycloalkyl, C₃₋₄ alkenyl, or C₃₋₄ alkynyl;
R₄ is —CH₂R₄ₐ, —CH₂CH₂R₄ₐ, —CH₂CHR₄ₐR_{4d}, —CHR₄ₐR_{4b}, or —CR₄ₐR_{4b}R_{4c};
R₄ₐ and R_{4b} are independently:
   (i) —CN or C₁₋₆ alkyl substituted with zero to 4 substituents independently selected from F, Cl, —CN, —OH, —OCH₃, —SCH₃, C₁₋₃ fluoroalkoxy, —NRₐRₐ, —S(O)₂Rₑ, or —NRₐS(O)₂Rₑ;
   (ii) C₃₋₆ cycloalkyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₄ hydroxyalkyl, —(CH₂)₁₋₂O(C₁₋₃ alkyl), C₁₋₄ alkoxy, C₁₋₃ fluoroalkoxy, C₁₋₃ cyanoalkoxy, —O(C₁₋₄ hydroxyalkyl), —O(CRₓRₓ)₁₋₃O(C₁₋₃ alkyl), C₁₋₃ fluoroalkoxy, —O(CH₂)₁₋₃NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —CH₂NRₐRₐ, —NRₐS(O)₂(C₁₋₃ alkyl), —NRₐC(O)(C₁₋₃ alkyl), —(CRₓRₓ)₀₋₂NRₐC(O)O(C₁₋₄ alkyl), —P(O)(C₁₋₃ alkyl)₂, —S(O)₂(C₁₋₃ alkyl), —(CRₓRₓ)₁₋₂(C₃₋₄ cycloalkyl), —(CRₓRₓ)₁₋₂(morpholinyl), —(CRₓRₓ)₁₋₂(difluoromorpholinyl), —(CRₓRₓ)₁₋₂(dimethylmorpholinyl), —(CRₓRₓ)₁₋₂(oxaazabicyclo[2.2.1]heptanyl), (CRₓRₓ)₁₋₂(oxaazaspiro[3.3]heptanyl), —(CRₓRₓ)₁₋₂(methylpiperazinonyl), —(CRₓRₓ)₁₋₂(acetylpiperazinyl), —(CRₓRₓ)₁₋₂(piperidinyl), (CRₓRₓ)₁₋₂(difluoropiperidinyl), —(CRₓRₓ)₁₋₂(methoxypiperidinyl), —(CRₓRₓ)₁₋₂(hydroxypiperidinyl), —O(CRₓRₓ)₀₋₂(C₃₋₆ cycloalkyl), —O(CRₓRₓ)₀₋₂(methylcyclopropyl), —O(CRₓRₓ)₀₋₂((ethoxycarbonyl)cyclopropyl), —O(CRₓRₓ)₀₋₂(oxetanyl), —O(CRₓRₓ)₀₋₂(methylazetidinyl), —O(CRₓRₓ)₀₋₂(tetrahydropyranyl), —O(CRₓRₓ)₁₋₂(morpholinyl), —O(CRₓRₓ)₀₋₂(thiazolyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, dioxolanyl, pyrrolidinonyl, and R_{d}; or
   (iii) C₁₋₄ alkyl substituted with one cyclic group selected from C₃₋₆ cycloalkyl, 4- to 10-membered heterocyclyl, mono- or bicyclic aryl, or 5- to 10-membered heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl;
or R₄ₐ and R_{4b} together with the carbon atom to which they are attached form a C₃₋₆ cycloalkyl or a 3- to 6-membered heterocyclyl, each substituted with zero to 3 R_{f};
each R_{f} is independently F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, and —NR_{c}R_{c};
R_{4c} is C₁₋₆ alkyl or C₃₋₆ cycloalkyl, each substituted with zero to 4 substituents independently selected from F, Cl, —OH, C₁₋₂ alkoxy, C₁₋₂ fluoroalkoxy, and —CN;
R₄ₐ is —OCH₃;
each R_{c} is independently H or C₁₋₂ alkyl;
R_{d} is phenyl substituted with zero to 1 substituent selected from F, Cl, —CN, —CH₃, and —OCH₃;
each R₅ is independently —CN, C₁₋₆ alkyl substituted with zero to 4 R_{g}, C₂₋₄ alkenyl substituted with zero to 4 R_{g}, C₂₋₄ alkynyl substituted with zero to 4 R_{g}, C₃₋₄ cycloalkyl substituted with zero to 4 R_{g}, phenyl substituted with zero to 4 R_{g}, oxadiazolyl substituted with zero to 3 R_{g}, pyridinyl substituted with zero to 4 R_{g}, —(CH₂)₁₋₂(4- to 10-membered heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each R_{g} is independently F, Cl, —CN, —OH, C₁₋₃ alkoxy, C₁₋₃ fluoroalkoxy, —O(CH₂)₁₋₂O(C₁₋₂ alkyl), or —NR_{c}R_{c};
m is zero, 1, 2, or 3; and
n is zero, 1, or 2.
58. The method of embodiment 57, wherein the inhibitor is a compound of Formula (II) or a pharmaceutically acceptable salt thereof, wherein:
R₁ is H, F, Cl, Br, —CN, —OH, C₁₋₃ alkyl substituted with zero to 4 R₁ₐ, cyclopropyl substituted with zero to 3 R₁ₐ, C₁₋₃ alkoxy substituted with zero to 3 R₁ₐ, —NRₐRₐ, —S(O)ₙCH₃, or —P(O)(CH₃)₂;
R₂ is H or C₁₋₂ alkyl substituted with zero to 2 R₂ₐ;
each R₂ₐ is independently F, Cl, —CN, —OH, —O(C₁₋₂ alkyl), cyclopropyl, C₃₋₄ alkenyl, or C₃₋₄ alkynyl;
R₄ₐ and R_{4b} are independently:
(i) —CN or C₁₋₄ alkyl substituted with zero to 4 substituents independently selected from F, Cl, —CN, —OH, —OCH₃, —SCH₃, C₁₋₃ fluoroalkoxy, and —NRₐRₐ;
(ii) C₃₋₆ cycloalkyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₂ hydroxyalkyl, —CH₂NRₐRₐ, —(CH₂)₁₋₂O(C₁₋₂ alkyl), —(CH₂)₁₋₂NRₓC(O)O(C₁₋₂ alkyl), C₁₋₄ alkoxy, —O(C₁₋₄ hydroxyalkyl), —O(CRₓRₓ)₁₋₂O(C₁₋₂ alkyl), C₁₋₃ fluoroalkoxy, C₁₋₃ cyanoalkoxy, —O(CH₂)₁₋₂NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —(CH₂)₁₋₂(C₃₋₄ cycloalkyl), —CRₓRₓ(morpholinyl), —CRₓRₓ(difluoromorpholinyl), —CRₓRₓ(dimethylmorpholinyl), —CRₓRₓ(oxaazabicyclo[2.2.1]heptanyl), —CRₓRₓ(oxaazaspiro[3.3]heptanyl), —CRₓRₓ(methylpiperazinonyl), —CRₓRₓ(acetylpiperazinyl), —CRₓRₓ(piperidinyl), —CRₓRₓ(difluoropiperidinyl), —CRₓRₓ(methoxypiperidinyl), —CRₓRₓ(hydroxypiperidinyl), —O(CH₂)₀₋₂(C₃₋₄ cycloalkyl), —O(CH₂)₀₋₂(methylcyclopropyl), —O(CH₂)₀₋₂((ethoxycarbonyl)cyclopropyl), —O(CH₂)₀₋₂(oxetanyl), —O(CH₂)₀₋₂(methylazetidinyl), —O(CH₂)₁₋₂(morpholinyl), —O(CH₂)₀₋₂(tetrahydropyranyl), —O(CH₂)₀₋₂(thiazolyl), cyclopropyl, cyanocyclopropyl, methylazetidinyl, acetylazetidinyl, (tert-butoxycarbonyl)azetidinyl, dioxolanyl, pyrrolidinonyl, triazolyl, tetrahydropyranyl, morpholinyl, thiophenyl, methylpiperidinyl, and R_{d}; or
(iii) C₁₋₃ alkyl substituted with one cyclic group selected from C₃₋₆ cycloalkyl, 4- to 10-membered heterocyclyl, mono- or bicyclic aryl, or 5- to 10-membered heteroaryl, said cyclic group substituted with zero to 3 substituents independently selected from F, Cl, Br, —OH, —CN, C₁₋₃ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₄ cycloalkyl;
or R₄ₐ and R_{4b} together with the carbon atom to which they are attached, form a C₃₋₆ cycloalkyl or a 3- to 6-membered heterocyclyl, each substituted with zero to 3 R_{f};
each R_{f} is independently F, Cl, Br, —OH, —CN, C₁₋₄ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₄ alkyl, C₁₋₂ fluoroalkyl, C₁₋₃ alkoxy, C₁₋₂ fluoroalkoxy, and —NR_{c}R_{c};
R_{4c} is C₁₋₄ alkyl or C₃₋₆ cycloalkyl, each substituted with zero to 4 substituents independently selected from F, Cl, —OH, C₁₋₂ alkoxy, C₁₋₂ fluoroalkoxy, and —CN;
each R₅ is independently —CN, C₁₋₅ alkyl substituted with zero to 4 R_{g}, C₂₋₃ alkenyl substituted with zero to 4 R_{g}, C₂₋₃ alkynyl substituted with zero to 4 R_{g}, C₃₋₄ cycloalkyl substituted with zero to 4 R_{g}, phenyl substituted with zero to 3 R_{g}, oxadiazolyl substituted with zero to 3 R_{g}, pyridinyl substituted with zero to 3 R_{g}, —(CH₂)₁₋₂(4- to 10-membered heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each Rₓ is independently H or —CH₃; and
m is 1, 2, or 3.
59. The method of embodiment 58, wherein the inhibitor is a compound of Formula (II) or a pharmaceutically acceptable salt thereof having the structure:

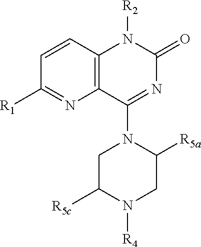

R₁ is —CN;
R₂ is —CH₃;
R₅ₐ is —CH₃ or —CH₂CH₃; and
R_{5c} is —CH₃, —CH₂CH₃, or —CH₂CH₂CH₃.
60. The method of embodiment 57, wherein the inhibitor is a compound of Formula (II) or a pharmaceutically acceptable salt thereof having the structure:

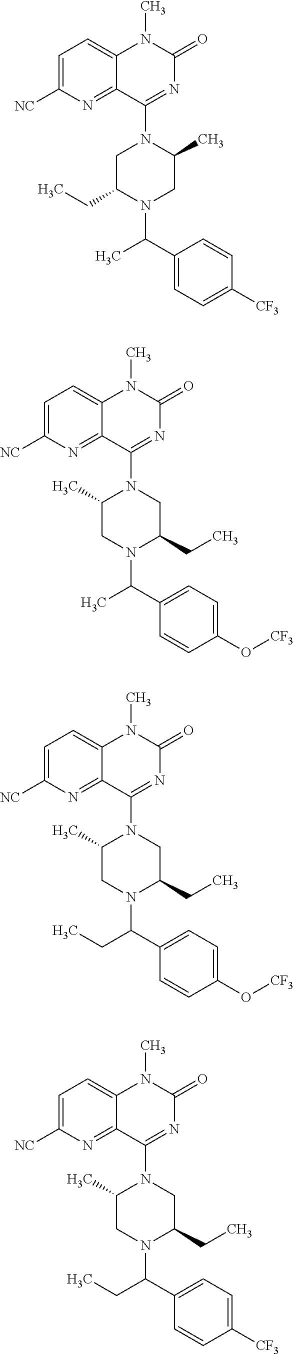 [SMILES: CC[C@@H]1CN(c2nc(=O)n(C)c3ccc(C#N)nc23)[C@@H](C)CN1C(C)c1ccc(C(F)(F)F)cc1 ; CC[C@@H]1CN(c2nc(=O)n(C)c3ccc(C#N)nc23)[C@@H](C)CN1C(C)c1ccc(OC(F)(F)F)cc1 ; CCC(c1ccc(OC(F)(F)F)cc1)N1C[C@H](C)N(c2nc(=O)n(C)c3ccc(C#N)nc23)C[C@H]1CC ; CCC(c1ccc(C(F)(F)F)cc1)N1C[C@H](C)N(c2nc(=O)n(C)c3ccc(C#N)nc23)C[C@H]1CC]

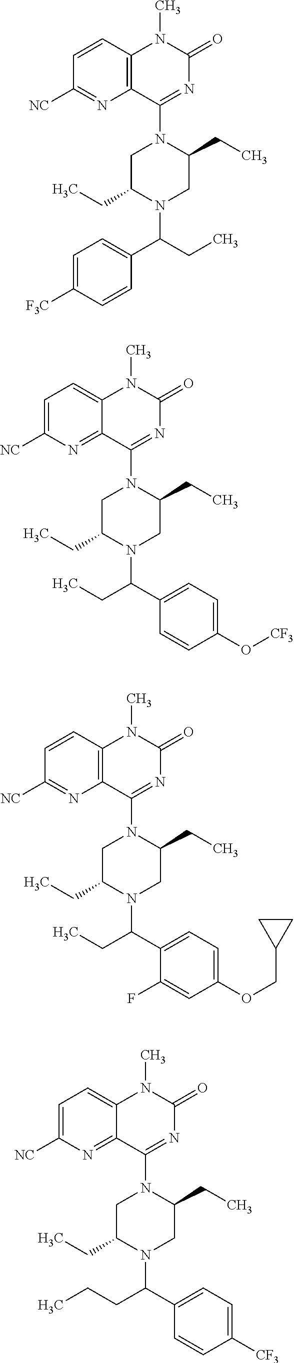 [SMILES: CCC(c1ccc(C(F)(F)F)cc1)N1C[C@H](CC)N(c2nc(=O)n(C)c3ccc(C#N)nc23)C[C@H]1CC ; CCC(c1ccc(OC(F)(F)F)cc1)N1C[C@H](CC)N(c2nc(=O)n(C)c3ccc(C#N)nc23)C[C@H]1CC ; CCC(c1ccc(OCC2CC2)cc1F)N1C[C@H](CC)N(c2nc(=O)n(C)c3ccc(C#N)nc23)C[C@H]1CC ; CCCC(c1ccc(C(F)(F)F)cc1)N1C[C@H](CC)N(c2nc(=O)n(C)c3ccc(C#N)nc23)C[C@H]1CC]

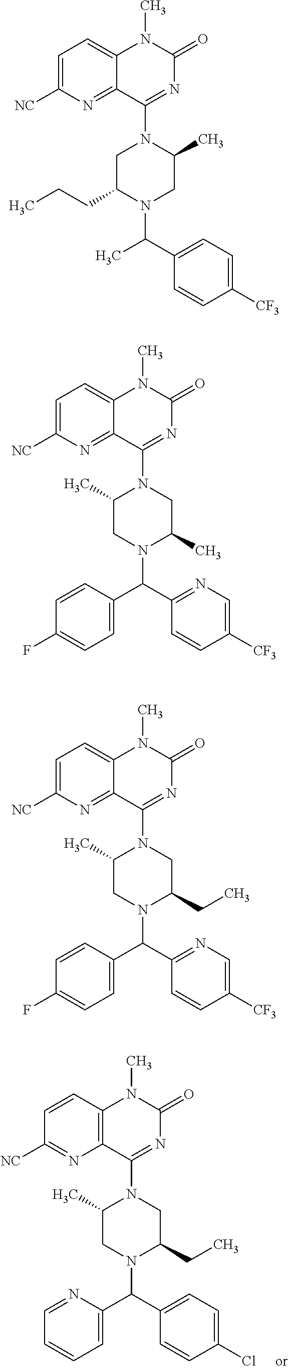 [SMILES: CCC[C@@H]1CN(c2nc(=O)n(C)c3ccc(C#N)nc23)[C@@H](C)CN1C(C)c1ccc(C(F)(F)F)cc1 ; C[C@@H]1CN(c2nc(=O)n(C)c3ccc(C#N)nc23)[C@@H](C)CN1C(c1ccc(F)cc1)c1ccc(C(F)(F)F)cn1 ; CC[C@@H]1CN(c2nc(=O)n(C)c3ccc(C#N)nc23)[C@@H](C)CN1C(c1ccc(F)cc1)c1ccc(C(F)(F)F)cn1 ; CC[C@@H]1CN(c2nc(=O)n(C)c3ccc(C#N)nc23)[C@@H](C)CN1C(c1ccc(Cl)cc1)c1ccccn1]

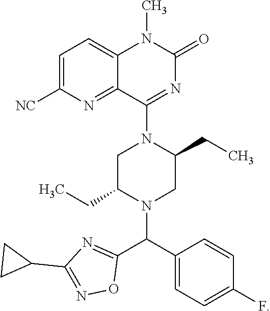 [SMILES: CC[C@H]1CN(C(c2ccc(F)cc2)c2nc(C3CC3)no2)[C@H](CC)CN1c1nc(=O)n(C)c2ccc(C#N)nc12]

61. The method of embodiment 57, wherein the inhibitor is a compound of Formula (II) or a pharmaceutically acceptable salt thereof having the structure:

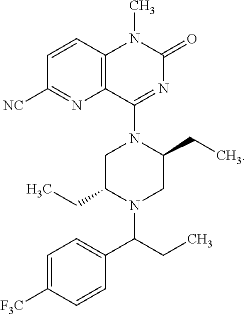 [SMILES: CCC(c1ccc(C(F)(F)F)cc1)N1C[C@H](CC)N(c2nc(=O)n(C)c3ccc(C#N)nc23)C[C@H]1CC]

62. The method of any one of embodiments 1-13 and 21-61, wherein the inhibitor is administered in a therapeutically effective amount.
63. The method of embodiment 62, wherein the therapeutically effective dose provides a dose of the inhibitor that is continuously therapeutically effective for the period of time that the inhibitor is administered.
64. The method of any one of embodiments 1-63, wherein the inhibitor is administered in a per day amount between or between about 0.25 and 250 mg, inclusive.
65. The method of any one of embodiments 1-64, wherein the inhibitor is administered in a per day amount between or between about 0.5 and 100 mg, inclusive.
66. The method of any one of embodiments 1-65, wherein the inhibitor is administered orally.
67. The method of any one of embodiments 1-66, wherein the recombinant receptor is an engineered T cell receptor (eTCR).
68. The method of any one of embodiments 1-66, wherein the recombinant receptor is a chimeric antigen receptor (CAR).
69. The method of embodiment 68, wherein the antigen is CD19, and the CAR is an anti-CD19 CAR.
70. The method of embodiment 68 or embodiment 69, wherein the CAR is the CAR of BREYANZI® (lisocabtagene maraleucel), TECARTUS™ (brexucabtagene autoleucel), KYMRIAH™ (tisagenlecleucel), or YESCARTA™ (axicabtagene ciloleucel).
71. The method of any one of embodiments 68-70, wherein the T cell therapy is BREYANZI® (lisocabtagene maraleucel), TECARTUS™ (brexucabtagene autoleucel), KYMRIAH™ (tisagenlecleucel), or YESCARTA™ (axicabtagene ciloleucel).
72. The method of embodiment 68, wherein the antigen is BCMA, and the CAR is an anti-BCMA CAR.
73. The method of embodiment 68 or embodiment 72, wherein the CAR is the CAR of ABECMA® (idecabtagene vicleucel) or CARVYKTI™ (ciltacabtagene autoleucel).
74. The method of any one of embodiments 68, 72, and 73, wherein the T cell therapy is ABECMA® (idecabtagene vicleucel) or CARVYKTI™ (ciltacabtagene autoleucel).
75. The method of any one of embodiments 1-74, wherein the cancer is a solid tumor.
76. The method of any one of embodiments 1-74, wherein the cancer is a hematological (liquid) tumor.
77. The method of any one of embodiments 1-76, wherein the cancer is a B cell malignancy.
78. The method of any one of embodiments 1-74, 76, and 77, wherein the cancer is a leukemia.
79. The method of any one of embodiments 1-74, 76, and 77, wherein the cancer is a lymphoma.
80. The method of any one of embodiments 1-74, 76, and 77, wherein the cancer is a myeloma.
81. The method of embodiment 80, wherein the myeloma is multiple myeloma.
82. The method of any one of embodiments 1-81, wherein the cancer is relapsed or refractory.
83. The method of any one of embodiments 1-82, wherein the T cell therapy comprises between or between about 0.1×10⁶ and 1,000×10⁶ total recombinant receptor-expressing T cells, inclusive.
84. The method of any one of embodiments 1-83, wherein the T cell therapy comprises between or between about 10×10⁶ and 1,000×10⁶ total recombinant receptor-expressing T cells, inclusive.
85. The method of any one of embodiments 1-84, wherein the T cell therapy comprises between or between about 10×10⁶ and 500×10⁶ total recombinant receptor-expressing T cells, inclusive.
86. The method of any of embodiments 83-85, wherein the recombinant receptor-expressing T cells are viable recombinant receptor-expressing T cells.
87. The method of any one of embodiments 1-86, wherein the T cell therapy is administered intravenously.
88. The method of any one of embodiments 1-87, wherein the T cells of the T cell therapy are autologous to the subject.
89. The method of any one of embodiments 1-87, wherein the T cells of the T cell therapy are allogeneic to the subject.
90. The method of any one of embodiments 1-89, wherein the T cells of the T cell therapy are human T cells.

### VIII. EXAMPLES

The following examples are included for illustrative purposes only and are not intended to limit the scope of the invention.

### Example 1: Synthesis of DGK Inhibitors

### DGKi Compound 1

### 4-((2R,5S)-4-(bis(4-fluorophenyl)methyl)-2,5-dimethylpiperazin-1-yl)-6-bromo-1-methyl-2-oxo-1,2-dihydro-1,5-naphthyridine-3-carbonitrile

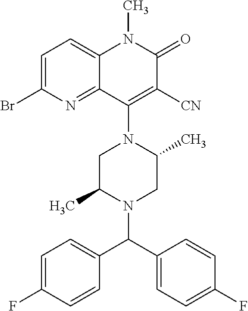 [SMILES: C[C@@H]1CN(C(c2ccc(F)cc2)c2ccc(F)cc2)[C@@H](C)CN1c1c(C#N)c(=O)n(C)c2ccc(Br)nc12]

### DGKi Compound 2

### Methyl 1-(bis(4-fluorophenyl)methyl)-4-(6-cyano-1-methyl-2-oxo-1,2-dihydro-1,5-naphthyridin-4-yl)piperazine-2-carboxylate

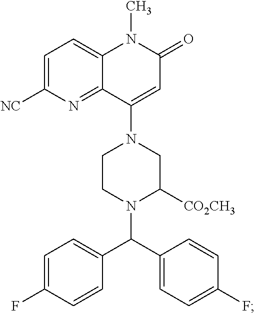 [SMILES: COC(=O)C1CN(c2cc(=O)n(C)c3ccc(C#N)nc23)CCN1C(c1ccc(F)cc1)c1ccc(F)cc1]

### DGKi Compound 3

### (R)-4-(4-(bis(4-fluorophenyl)methyl)-3-methylpiperazin-1-yl)-6-bromo-1-methyl-2-oxo-1,2-dihydro-1,5-naphthyridine-3-carbonitrile

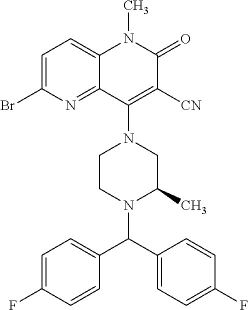 [SMILES: C[C@@H]1CN(c2c(C#N)c(=O)n(C)c3ccc(Br)nc23)CCN1C(c1ccc(F)cc1)c1ccc(F)cc1]

### DGKi Compound 4

### (R)-8-(4-(bis(4-fluorophenyl)methyl)-3-methylpiperazin-1-yl)-5-methyl-6-oxo-5,6-dihydro-1,5-naphthyridine-2,7-dicarbonitrile

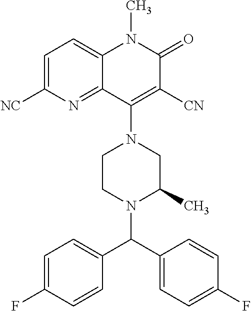 [SMILES: C[C@@H]1CN(c2c(C#N)c(=O)n(C)c3ccc(C#N)nc23)CCN1C(c1ccc(F)cc1)c1ccc(F)cc1]

### DGKi Compound 5

### 8-[(2S,5R)-4-[(4-fluorophenyl) (phenyl)methyl]-2,5-dimethylpiperazin-1-yl]-5-methyl-6-oxo-5,6-dihydro-1,5-naphthyridine-2-carbonitrile

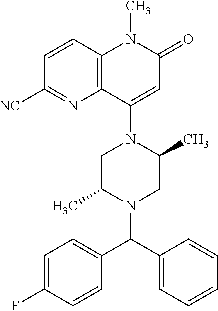 [SMILES: C[C@@H]1CN(c2cc(=O)n(C)c3ccc(C#N)nc23)[C@@H](C)CN1C(c1ccccc1)c1ccc(F)cc1]

### DGKi Compounds 6 and 7

### 8-[(2S,5R)-4-[(4-fluorophenyl) (phenyl)methyl]-2,5-dimethylpiperazin-1-yl]-5-methyl-6-oxo-5,6-dihydro-1,5-naphthyridine-2-carbonitrile

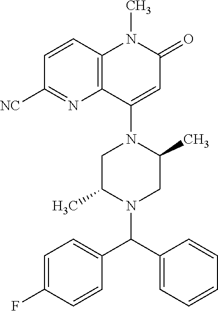 [SMILES: C[C@@H]1CN(c2cc(=O)n(C)c3ccc(C#N)nc23)[C@@H](C)CN1C(c1ccccc1)c1ccc(F)cc1]

### DGKi Compound 8

### 4-[(2S,5R)-4-[(4-chlorophenyl)(4-fluorophenyl)methyl]-2,5-dimethylpiperazin-1-yl]-6-methoxy-1-methyl-1,2-dihydro-1,5-naphthyridin-2-one

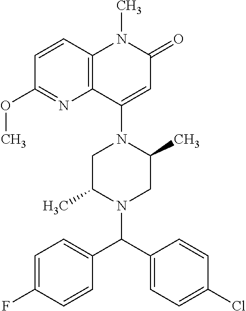 [SMILES: COc1ccc2c(n1)c(N1C[C@@H](C)N(C(c3ccc(F)cc3)c3ccc(Cl)cc3)C[C@@H]1C)cc(=O)n2C]

### DGKi Compound 9

### 8-[(2S,5R)-4-{[2-(difluoromethyl)-4-fluorophenyl]methyl}-2,5-dimethylpiperazin-1-yl]-5-methyl-6-oxo-5,6-dihydro-1,5-naphthyridine-2-carbonitrile

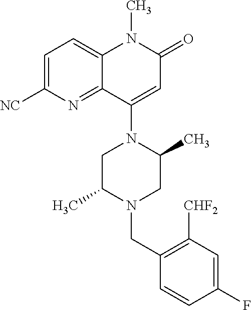 [SMILES: C[C@@H]1CN(c2cc(=O)n(C)c3ccc(C#N)nc23)[C@@H](C)CN1Cc1ccc(F)cc1C(F)F]

### DGKi Compound 10

### 8-[(2S,5R)-4-[(4-fluorophenyl)(4-methylphenyl)methyl]-2,5-dimethylpiperazin-1-yl]-5-methyl-6-oxo-5,6-dihydro-1,5-naphthyridine-2-carbonitrile

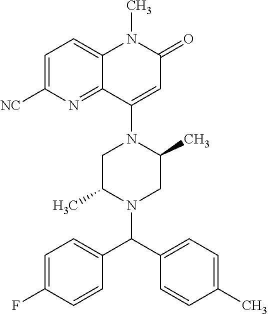 [SMILES: Cc1ccc(C(c2ccc(F)cc2)N2C[C@H](C)N(c3cc(=O)n(C)c4ccc(C#N)nc34)C[C@H]2C)cc1]

### DGKi Compound 11

### 8-[(2S,5R)-4-[1-(2,6-difluorophenyl)ethyl]-2,5-dimethylpiperazin-1-yl]-5-methyl-6-oxo-5,6-dihydro-1,5-naphthyridine-2-carbonitrile

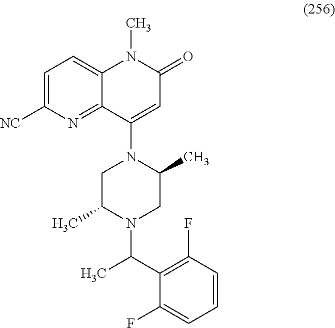 [SMILES: CC(c1c(F)cccc1F)N1C[C@H](C)N(c2cc(=O)n(C)c3ccc(C#N)nc23)C[C@H]1C]

### DGKi Compounds 12-14

### 8-((2S,5R)-4-(1-(2,4-difluorophenyl)propyl)-2,5-dimethylpiperazin-1-yl)-5-methyl-6-oxo-5,6-dihydro-1,5-naphthyridine-2-carbonitrile

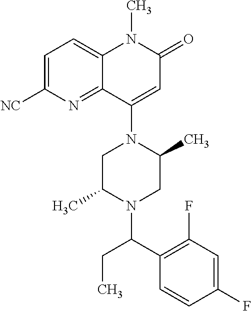 [SMILES: CCC(c1ccc(F)cc1F)N1C[C@H](C)N(c2cc(=O)n(C)c3ccc(C#N)nc23)C[C@H]1C]

### Intermediate 1

### Ethyl 6-cyano-3-(N-methylacetamido)picolinate

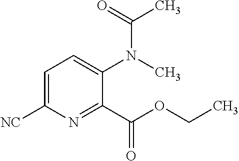 [SMILES: CCOC(=O)c1nc(C#N)ccc1N(C)C(C)=O]

To a stirred pale yellow solution of ethyl 3-(N-methylacetamido)-1-(11-oxidanyl)-114-pyridine-2-carboxylate (50 g, 210 mmol) in DCM (500 mL) at room temperature was added trimethylsilyl cyanide (39.4 mL, 294 mmol). The reaction mixture was stirred for 10 min and cooled the mixture to −10 °C. Next, benzoyl chloride (34.1 mL, 294 mmol) was added through a 50 mL addition funnel over 15 min followed by TEA (41.0 mL, 294 mmol) through a 50 mL addition funnel slowly over 20 min. An exothermic reaction was observed during TEA addition. The reaction mixture turned to a turbid mixture (TEA salt) which was stirred for 2.5 h at the same temperature. The reaction was quenched with 10% NaHCO₃ solution (500 mL) and extracted with DCM (3×300 mL). The combined organic solution was washed with brine (2×250 mL) then dried over Na₂SO₄ and concentrated to yield a light yellow crude material. The crude material was purified through normal phase RediSep silica column on ISCO® using EA/petroleum ether as eluent. The product was isolated by 65-70% EA/petroleum ether, fractions were concentrated to afford ethyl 6-cyano-3-(N-methylacetamido)picolinate (43 g, 83% yield) as a light brown liquid; LCMS: m/z=248.0 (M+H); rt 1.255 min; LC-MS Method: Column-KINETEX-XB-C18 (75×3 mm-2.6 μm); Mobile phase A: 10 mM ammonium formate in water:acetonitrile (98:2); Mobile phase B: 10 mM ammonium formate in water:acetonitrile (2:98); Gradient: 20-100% B over 4 minutes, flow rate 1.0 mL/min, then a 0.6 minute hold at 100% B flow rate 1.5 mL/min; then Gradient: 100-20% B over 0.1 minutes, flow rate 1.5 mL/min.

### Intermediate 2

### 8-Hydroxy-5-methyl-6-oxo-5,6-dihydro-1,5-naphthyridine-2-carbonitrile

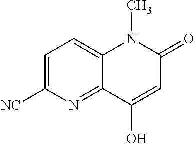

To a stirred solution of ethyl 6-cyano-3-(N-methylacetamido)picolinate (0.9 g, 3.64 mmol) in tetrahydrofuran (10 mL) was added KHMDS (4.80 mL, 4.37 mmol) at −78 °C over 10 min. The reaction mixture was stirred for 15 min. The reaction mixture was slowly warmed to room temperature over 30 min and then stirred for another 90 min. The reaction mixture was cooled to 0 °C. The reaction was quenched with saturated sodium bicarbonate solution (70 mL). The mixture was diluted with ethyl acetate (2×100 mL). The aqueous layer was collected and acidified with 1.5 N HCL to adjust the pH to ~3.0. The mixture was stirred for 15 min to form a solid mass, which was filtered through a Buchner funnel to yield 8-hydroxy-5-methyl-6-oxo-5,6-dihydro-1,5-naphthyridine-2-carbonitrile 550 mg, 75% yield) as a brown solid. LCMS: m/z=202.0 (M+H); rt 0.361 min; LC-MS Method: Column-KINETEX-XB-C18 (75×3 mm-2.6 μm); Mobile phase A: 10 mM ammonium formate in water:acetonitrile (98:2); Mobile phase B: 10 mM ammonium formate in water:acetonitrile (2:98); Gradient: 20-100% B over 4 minutes, flow rate 1.0 mL/min, then a 0.6 minute hold at 100% B flow rate 1.5 mL/min; then Gradient: 100-20% B over 0.1 minutes, flow rate 1.5 mL/min.

### Intermediate 3

### 8-Chloro-5-methyl-6-oxo-5,6-dihydro-1,5-naphthyridine-2-carbonitrile

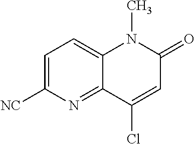

To a stirred solution of 8-hydroxy-5-methyl-6-oxo-5,6-dihydro-1,5-naphthyridine-2-carbonitrile (0.55 g, 2.73 mmol) in acetonitrile (10 mL) was added POCl₃(1.53 mL, 16.4 mmol). The reaction mixture was heated up to 85 °C over 5 min and was stirred for 16 h. The reaction mixture was concentrated under reduced pressure to yield crude material. The reaction mixture was cooled to 0 °C. The reaction was quenched with saturated sodium bicarbonate solution (50 mL). The reaction was diluted with DCM (3×100 mL). The combined organic layer was dried over anhydrous sodium sulfate, filtered, and evaporated under reduced pressure to yield 8-chloro-5-methyl-6-oxo-5,6-dihydro-1,5-naphthyridine-2-carbonitrile (0.25 g, 29.1% yield) as a brown solid. LCMS: m/z=220.2 (M+H); rt 1.528 min; LC-MS Method: Column-KINETEX-XB-C18 (75×3 mm-2.6 μm); Mobile phase A: 10 mM ammonium formate in water:acetonitrile (98:2); Mobile phase B: 10 mM ammonium formate in water:acetonitrile (2:98); Gradient: 20-100% B over 4 minutes, flow rate 1.0 mL/min, then a 0.6 minute hold at 100% B flow rate 1.5 mL/min; then Gradient: 100-20% B over 0.1 minutes, flow rate 1.5 mL/min.

### Intermediate 4

### Stereochemistry: A

### (Cyanomethyl)trimethylphosphonium Iodide

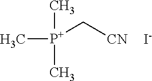

(Cyanomethyl)trimethylphosphonium iodide was prepared according to the general method described in Zaragoza, F., et al., J. Org. Chem. 2001, 66, 2518-2521. In a 1 L round bottom flask, trimethylphosphine in toluene (100 mL, 100 mmol) was diluted with THF (50.0 mL) and toluene (50.0 mL), and cooled on an ice bath. The reaction mixture was stirred vigorously while iodoacetonitrile (7 mL, 16.7 g, 68.3 mmol) was added dropwise to produce a tan precipitate. The cooling bath was removed and the reaction mixture was stirred overnight at room temperature. The flask was placed in a sonicator to break up any clumped solids. The reaction mixture was stirred an additional 4 hours. The solids were collected by filtration and dried under vacuum to give (cyanomethyl)trimethylphosphonium iodide (16.6 g, 68.3 mmol, 68.3% yield). ¹H NMR (400 MHz, DMSO-d₆) δ 4.03 (d, J=16.4 Hz, 2H), 2.05 (d, J=15.4 Hz, 9H).

### Intermediate 5

### Stereochemistry: Homochiral

### 8-((2S,5R)-2,5-Dimethylpiperazin-1-yl)-5-methyl-6-oxo-5,6-dihydro-1,5-naphthyridine-2-carbonitrile, TFA

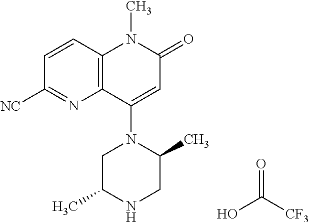 [SMILES: C[C@@H]1CN(c2cc(=O)n(C)c3ccc(C#N)nc23)[C@@H](C)CN1 ; O=C(O)C(F)(F)F]

To a solution of 6-cyano-1-methyl-2-oxo-1,2-dihydro-1,5-naphthyridin-4-yl trifluoromethanesulfonate (65 g, 195 mmol) and tert-butyl(2R,5S)-2,5-dimethylpiperazine-1-carboxylate (43.9 g, 205 mmol) in acetonitrile (1.3 L) was added DIPEA (0.102 L, 585 mmol). The solution was stirred at 80 °C for 6 hours. The solvent was removed and the crude residue chromatographed on silica gel (product Rf 0.4 in 100% ethyl acetate). The product, tert-butyl (2R,5S)-4-(6-cyano-1-methyl-2-oxo-1,2-dihydro-1,5-naphthyridin-4-yl)-2,5-dimethylpiperazine-1-carboxylate (75 g, 189 mmol, 97% yield) was obtained. LCMS: m/z=398.2 (M+H); rt 2.7 min. Method: Column-Kinetex XB-C18 (75×3 mm-2.6 μm), flow rate 1 mL/min; gradient time 4 min; 20% Solvent B to 100% Solvent B; monitoring at 254 nm (Solvent A: 98% water: 2% acetonitrile; 10 mM ammonium formate; Solvent B: 2% water: 98% acetonitrile; 10 mM ammonium formate.

To a solution of tert-butyl(2R,5S)-4-(6-cyano-1-methyl-2-oxo-1,2-dihydro-1,5-naphthyridin-4-yl)-2,5-dimethylpiperazine-1-carboxylate (30 g, 75 mmol) in ethyl acetate (1000 mL) at 0 °C was added HCl (4 M in dioxane) (189 mL, 755 mmol) and the temperature was allowed to reach room temperature while stirring for 6 h. LC/MS analysis showed ~90% product mass at 0.60 RT along with ~4% of an amide byproduct mass (consistent with nitrile hydrolysis) at 0.44 RT. The reaction mixture was diluted with methyl t-butyl ether (MTBE, 2000 mL), stirred for 15 mins, and the HCl salt of product was filtered, washed with MTBE (100 ml). The HCl salt was dissolved in water (300 ml) and the pH adjusted to~8 using 10% aqueous sodium bicarbonate. The organic portion was extracted with DCM (5×250 ml). The combined organic layers were washed with water (2×300 mL), dried over sodium sulphate and concentrated to afford 8-((2S,5R)-2,5-dimethylpiperazin-1-yl)-5-methyl-6-oxo-5,6-dihydro-1,5-naphthyridine-2-carbonitrile (20 g, 65.2 mmol, 86% yield). LCMS: m/z=298.2 (M+H); rt 0.5 min. Method: Column-Kinetex XB-C18 (75×3 mm-2.6 μm), flow rate 1 mL/min; gradient time 4 min; 20% Solvent B to 100% Solvent B; monitoring at 254 nm (Solvent A: 98% water: 2% acetonitrile; 10 mM ammonium formate; Solvent B: 2% water: 98% acetonitrile; 10 mM ammonium formate. ¹H NMR (400 MHz, CDCl₃) δ 7.79 (d, J=8.8 Hz, 1H), 7.70 (d, J=12, 3.2 Hz, 1H), 6.29 (s, 1H), 3.80 (dd, J=8.8 Hz, 1H) 3.70 (m, 1H), 3.65 (s, 3H), 3.29 (m, 2H), 2.80 (m, 2H), 1.19 (d, J=6 Hz, 3H), 1.15 (d, J=6 Hz, 3H). ¹³C NMR (75 MHz, Chloroform-d) δ 161.9, 155.0, 138.5, 137.0, 128.2, 125.0, 122.2, 117.2, 111.3, 56.5, 51.9, 50.0, 49.5, 29.0, 18.8, 15.4.

### Intermediate 6

### 8-Chloro-5-methyl-7-nitro-6-oxo-5,6-dihydro-1,5-naphthyridine-2-carbonitrile

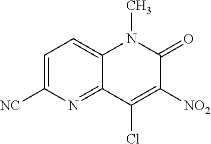 [SMILES: Cn1c(=O)c([N+](=O)[O-])c(Cl)c2nc(C#N)ccc21]

In a 2 dram vial containing 8-hydroxy-5-methyl-7-nitro-6-oxo-5,6-dihydro-1,5-naphthyridine-2-carbonitrile (192 mg, 0.780 mmol) a magnetic stir bar and acetonitrile (3.1 mL) were added. Next, DIEA (0.272 mL, 1.560 mmol) was added to the suspension. The reaction mixture was stirred for 1-2 minutes until the reaction mixture became a homogeneous yellow solution. To the reaction mixture was added phosphorous oxychloride (0.131 mL, 1.404 mmol). The vial was capped under nitrogen with vent to an oil bubbler. The reaction mixture was stirred at room temperature for 1.5 hours then benzyltriethylammonium chloride (200 mg, 0.878 mmol) was added to the reaction mixture. The vial was capped under a nitrogen atmosphere and immersed in an oil bath (65 °C) and heated for 1 hour. The reaction mixture was cooled and the reaction volatiles were remove in vacuo using a rotary evaporator. The reaction residue was dissolved in ethyl acetate, poured into a beaker containing ice (~10 mL), and then transferred to a separatory funnel. The aqueous phase was extracted with ethyl acetate. The organic extracts combined and washed sequentially with 1.5 M K₂HPO₄, saturated aqueous sodium bicarbonate, and brine. The organic extract was dried over magnesium sulfate, filtered, and solvent removed in vacuo to give a 204 mg of a brownish crystalline solid. LCMS: Column: Waters Acquity UPLC BEH C18, 2.1×50 mm, 1.7 μm particles; Mobile Phase A: 100% water with 0.05% trifluoroacetic acid; Mobile Phase B: 100% acetonitrile with 0.05% trifluoroacetic acid; Temperature: 40 °C; Gradient: 2-98% B over 1.5 minutes, then a 0.5 minute hold at 98% B; Flow: 0.8 mL/min; Detection: UV at 220 nm. Retention Time=1.01 min.; Obs. Adducts: [M+H]; Obs. Masses: 265.0 (weak ionization). ¹H NMR (CHLOROFORM-d) δ 8.03 (d, J=8.8 Hz, 1H), 7.89-7.97 (m, 1H), 3.82 (s, 3H).

### Intermediate 7

### 8-((2S,5R)-2,5-Dimethylpiperazin-1-yl)-5-methyl-6-oxo-5,6-dihydro-1,5-naphthyridine-2-carbonitrile, TFA

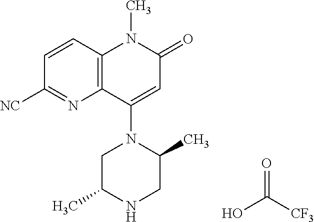 [SMILES: C[C@@H]1CN(c2cc(=O)n(C)c3ccc(C#N)nc23)[C@@H](C)CN1 ; O=C(O)C(F)(F)F]

To a solution of 6-cyano-1-methyl-2-oxo-1,2-dihydro-1,5-naphthyridin-4-yl trifluoromethanesulfonate (65 g, 195 mmol) and tert-butyl(2R,5S)-2,5-dimethylpiperazine-1-carboxylate (43.9 g, 205 mmol) in acetonitrile (1.3 L) was added DIPEA (0.102 L, 585 mmol). The solution was stirred at 80 °C for 6 hours. The solvent was removed and the crude residue chromatographed on silica gel (product Rf 0.4 in 100% ethyl acetate). The product, tert-butyl (2R,5S)-4-(6-cyano-1-methyl-2-oxo-1,2-dihydro-1,5-naphthyridin-4-yl)-2,5-dimethylpiperazine-1-carboxylate (75 g, 189 mmol, 97% yield) was obtained. LCMS: m/z=398.2 (M+H); rt 2.7 min. Method: Column-Kinetex XB-C18 (75×3 mm-2.6 μm), flow rate 1 mL/min; gradient time 4 min; 20% Solvent B to 100% Solvent B; monitoring at 254 nm (Solvent A: 98% water: 2% acetonitrile; 10 mM ammonium formate; Solvent B: 2% water: 98% acetonitrile; 10 mM ammonium formate.

To a solution of tert-butyl(2R,5S)-4-(6-cyano-1-methyl-2-oxo-1,2-dihydro-1,5-naphthyridin-4-yl)-2,5-dimethylpiperazine-1-carboxylate (30 g, 75 mmol) in ethyl acetate (1000 mL) at 0 °C was added HCl (4 M in dioxane) (189 mL, 755 mmol) and the temperature was allowed to reach room temperature while stirring for 6 h. LC/MS analysis showed ~90% product mass at 0.60 RT along with ~4% of an amide byproduct mass (consistent with nitrile hydrolysis) at 0.44 RT. The reaction mixture was diluted with methyl t-butyl ether (MTBE, 2000 mL), stirred for 15 mins, and the HCl salt of product was filtered, washed with MTBE (100 ml). The HCl salt was dissolved in water (300 ml) and the pH adjusted to ~8 using 10% aqueous sodium bicarbonate. The organic portion was extracted with DCM (5×250 mL). The combined organic layers were washed with water (2×300 mL), dried over sodium sulphate and concentrated to afford 8-((2S,5R)-2,5-dimethylpiperazin-1-yl)-5-methyl-6-oxo-5,6-dihydro-1,5-naphthyridine-2-carbonitrile (20 g, 65.2 mmol, 86% yield). LCMS: m/z=298.2 (M+H); rt 0.5 min. Method: Column-Kinetex XB-C18 (75×3 mm-2.6 μm), flow rate 1 mL/min; gradient time 4 min; 20% Solvent B to 100% Solvent B; monitoring at 254 nm (Solvent A: 98% water: 2% acetonitrile; 10 mM ammonium formate; Solvent B: 2% water: 98% acetonitrile; 10 mM ammonium formate. ¹H NMR (400 MHz, CDCl₃) δ 7.79 (d, J=8.8 Hz, 1H), 7.70 (d, J=12, 3.2 Hz, 1H), 6.29 (s, 1H), 3.80 (dd, J=8.8 Hz, 1H) 3.70 (m, 1H), 3.65 (s, 3H), 3.29 (m, 2H), 2.80 (m, 2H), 1.19 (d, J=6 Hz, 3H), 1.15 (d, J=6 Hz, 3H). ¹³C NMR (75 MHz, Chloroform-d) δ 161.9, 155.0, 138.5, 137.0, 128.2, 125.0, 122.2, 117.2, 111.3, 56.5, 51.9, 50.0, 49.5, 29.0, 18.8, 15.4. Stereochemistry: Homochiral.

### Method of Synthesizing DGKi Compound 1

### 4-((2R,5S)-4-(bis(4-fluorophenyl)methyl)-2,5-dimethylpiperazin-1-yl)-6-bromo-1-methyl-2-oxo-1,2-dihydro-1,5-naphthyridine-3-carbonitrile

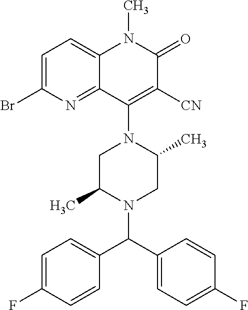 [SMILES: C[C@@H]1CN(C(c2ccc(F)cc2)c2ccc(F)cc2)[C@@H](C)CN1c1c(C#N)c(=O)n(C)c2ccc(Br)nc12]

To a stirred solution of 6-bromo-3-cyano-1-methyl-2-oxo-1,2-dihydro-1,5-naphthyridin-4-yl trifluoromethanesulfonate (80 mg, 0.194 mmol) in acetonitrile (5 mL) were added DIPEA (0.102 mL, 0.582 mmol) and HCl salt of (2S,5R)-1-(bis(4-fluorophenyl)methyl)-2,5-dimethylpiperazine (75 mg, 0.214 mmol). The reaction mixture was stirred at 85 °C overnight. The solvent was removed under reduced pressure and the residue was dissolved in ethyl acetate (15 mL). The organic layer was washed with brine, dried over Na₂SO₄ and concentrated under reduced pressure. The crude residue purified by silica gel column chromatography using 24 g flash column, eluting with 50-80% EtOAc in petroleum ether. The fractions were concentrated under reduced pressure to yield 4-((2R,5S)-4-(bis(4-fluorophenyl)methyl)-2,5-dimethylpiperazin-1-yl)-6-bromo-1-methyl-2-oxo-1,2-dihydro-1,5-naphthyridine-3-carbonitrile (95 mg, 85% yield); LCMS: m/z=578.2 (M+H); rt 3.916 min.

### Method of Synthesizing DGKi Compound 2

### Methyl 1-(bis(4-fluorophenyl)methyl)-4-(6-cyano-1-methyl-2-oxo-1,2-dihydro-1,5-naphthyridin-4-yl)piperazine-2-carboxylate

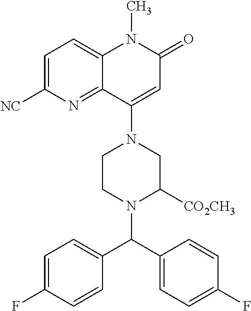 [SMILES: COC(=O)C1CN(c2cc(=O)n(C)c3ccc(C#N)nc23)CCN1C(c1ccc(F)cc1)c1ccc(F)cc1]

To a stirred solution of 8-chloro-5-methyl-6-oxo-5,6-dihydro-1,5-naphthyridine-2-carbonitrile (22.90 mg, 0.104 mmol) in DMA (1 mL) and t-butanol (4 mL) was added the TFA salt of methyl 1-(bis(4-fluorophenyl)methyl)piperazine-2-carboxylate (40 mg, 0.087 mmol) and cesium carbonate (85 mg, 0.261 mmol) under a nitrogen atmosphere, followed by the addition of chloro(2-dicyclohexylphosphino-2′,6′-diisopropoxy-1,1′-biphenyl) [2-(2′-amino-1,1′-biphenyl)]palladium(II) (3.37 mg, 4.34 μmol). The reaction vessel was immersed in an oil bath at 70 °C. The bath temperature was raised to 90 °C over 2 min and the reaction mixture was stirred for 16 h. The reaction mixture was filtered through a celite bed and was concentrated under high vacuum to yield a brown gum. The crude material was purified via preparative HPLC with the following conditions: Column: Sunfire C18, 19×150 mm, 5 μm particles; Mobile Phase A: 10 mM ammonium acetate pH 4.5 with acetic acid; Mobile Phase B: acetonitrile; Gradient: 30-100% B over 15 minutes, then a 5 minute hold at 100% B; Flow: 17 mL/min. Fractions containing the product were combined and dried via centrifugal evaporation to yield methyl 1-(bis(4-fluorophenyl)methyl)-4-(6-cyano-1-methyl-2-oxo-1,2-dihydro-1,5-naphthyridin-4-yl)piperazine-2-carboxylate (3.5 mg, 6.23 μmol, 7.17% yield). LCMS: m/z=530.2 (M+H); rt 2.20 min. LC-MS Method: Column-X Bridge BEH XP C18 (50×2.1 mm 2.5 μm; flow rate 1.1 mL/min; gradient time 3 min; Temperature: 50 °C, 0% Solvent B to 100% Solvent B; monitoring at 220 nm (Solvent A: 95% water: 5% acetonitrile; 10 mM ammonium acetate; Solvent B: 5% water: 95% acetonitrile; 10 mM ammonium acetate). ¹H NMR (400 MHz, DMSO-d₆) δ ppm 8.16 (d, J=8.8 Hz, 1H), 8.08 (d, J=9.0 Hz, 1H), 7.57 (dd, J=8.8, 5.6 Hz, 2H), 7.42-7.28 (m, 2H), 7.22-7.08 (m, 4H), 6.14 (s, 1H), 5.17 (s, 1H), 4.78 (d, J=12.2 Hz, 1H), 3.64 (d, J=12.0 Hz, 1H), 3.59 (s, 3H), 3.54 (s, 3H), 3.45-3.35 (m, 2H), 3.15 (dd, J=12.5, 3.9 Hz, 1H), 3.04 (td, J=11.7, 2.9 Hz, 1H), 2.71-2.63 (m, 1H).

### Method of Synthesizing DGKi Compound 3

### (R)-4-(4-(bis(4-fluorophenyl)methyl)-3-methylpiperazin-1-yl)-6-bromo-1-methyl-2-oxo-1,2-dihydro-1,5-naphthyridine-3-carbonitrile

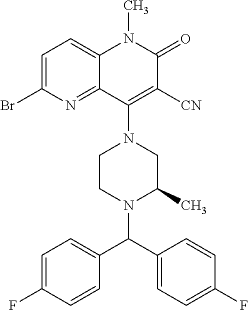 [SMILES: C[C@@H]1CN(c2c(C#N)c(=O)n(C)c3ccc(Br)nc23)CCN1C(c1ccc(F)cc1)c1ccc(F)cc1]

To a stirred solution of 6-bromo-3-cyano-1-methyl-2-oxo-1,2-dihydro-1,5-naphthyridin-4-yl trifluoromethanesulfonate (100 mg, 0.243 mmol) in acetonitrile (8 mL) were added DIPEA (0.127 mL, 0.728 mmol) and HCl salt of (R)-1-(bis(4-fluorophenyl)methyl)-2-methylpiperazine (82 mg, 0.243 mmol). The reaction mixture was heated up to 85 °C over 5 min and was stirred for 1 h. The reaction mixture was concentrated under high vacuum to yield a brown gum. The crude compound was purified by ISCO® using 12 g silica gel column; 60-67% ethyl acetate/petroleum ether to yield (R)-4-(4-(bis(4-fluorophenyl)methyl)-3-methylpiperazin-1-yl)-6-bromo-1-methyl-2-oxo-1,2-dihydro-1,5-naphthyridine-3-carbonitrile (90 mg, 42.7% yield) as a brown gum; LCMS: m/z=566.0 (M+2H); rt 2.23 min. LC-MS Method: Column-AQUITY UPLC BEH C18 (3.0×50 mm) 1.7 μm; Mobile phase A: Buffer: acetonitrile (95:5); Mobile phase B: Buffer: acetonitrile (5:95), Buffer: 10 mM ammonium acetate; Gradient: 20-100% B over 2.0 minutes, then a 0.2 minute hold at 100% B, flow rate 0.7 mL/min.

### Method of Synthesizing DGKi Compound 4

### (R)-8-(4-(bis(4-fluorophenyl)methyl)-3-methylpiperazin-1-yl)-5-methyl-6-oxo-5,6-dihydro-1,5-naphthyridine-2,7-dicarbonitrile

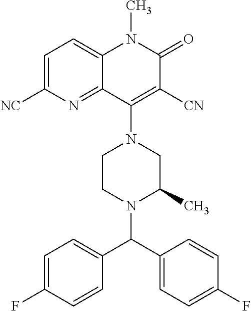 [SMILES: C[C@@H]1CN(c2c(C#N)c(=O)n(C)c3ccc(C#N)nc23)CCN1C(c1ccc(F)cc1)c1ccc(F)cc1]

To a stirred solution of (R)-4-(4-(bis(4-fluorophenyl)methyl)-3-methylpiperazin-1-yl)-6-bromo-1-methyl-2-oxo-1,2-dihydro-1,5-naphthyridine-3-carbonitrile (90 mg, 0.159 mmol) in NMP (5 mL) were added zinc (2.085 mg, 0.032 mmol) and zinc cyanide (37.4 mg, 0.319 mmol) under nitrogen. The nitrogen purging was continued for 3 min and dppf (5.30 mg, 9.57 μmol) and Pd₂(dba)₃(14.6 mg, 0.016 mmol) were added. The reaction mixture was heated up to 80 °C over 5 min and was stirred for 4 h. The reaction mixture was filtered through celite bed and was concentrated under high vacuum to yield a brown gum. The crude material was purified via preparative HPLC. HPLC Method: Column-SUNFIRE C18 (150 mm×19 mm ID, 5 μm); Mobile phase A: 10 mM Ammonium acetate in water; Mobile phase B: acetonitrile; Gradient: 40-60% B over 3.0 minutes, flow rate 17 mL/min, then a 17 minute hold at 60-100% B flow rate 17 mL/min. Fractions containing the product were combined and concentrated under high vacuum. Then sample was diluted with (EtOH\H₂O, 1:3) and was lyophilized overnight to yield (R)-8-(4-(bis(4-fluorophenyl)methyl)-3-methylpiperazin-1-yl)-5-methyl-6-oxo-5,6-dihydro-1,5-naphthyridine-2,7-dicarbonitrile (50 mg, 61.4% yield) as pale yellow solid. LCMS: m/z=511.2 (M+H); rt 3.520 min. LC-MS Method: Column-KINETEX-XB-C18 (75×3 mm-2.6μ); Mobile phase A: 10 mM ammonium formate in water:acetonitrile (98:2); Mobile phase B: 10 mM ammonium formate in water:acetonitrile (2:98); Gradient: 20-100% B over 4 minutes, flow rate 1.0 mL/min, then a 0.6 minute hold at 100% B flow rate 1.5 mL/min; then Gradient: 100-20% B over 0.1 minutes, flow rate 1.5 mL/min. ¹H NMR (400 MHz, DMSO-d₆) δ ppm 8.26 (d, J=8.8 Hz, 1H), 8.15 (d, J=9.0 Hz, 1H), 7.56 (dd, J=11.9, 8.7 Hz, 2H), 7.57 (dd, J=11.7, 8.8 Hz, 2H), 7.16 (t, J=8.9 Hz, 4H), 4.90 (s, 1H), 4.10 (d, J=13.0 Hz, 1H), 4.01 (d, J=12.5 Hz, 1H), 3.86 (dd, J=12.2, 2.9 Hz, 1H), 3.66-3.55 (m, 1H), 3.53 (s, 3H), 3.08-2.97 (m, 1H), 2.97-2.90 (m, 1H), 2.90 (s, 1H), 1.03 (d, J=6.6 Hz, 3H).

### Method of Synthesizing DGKi Compound 5

### 8-[(2S,5R)-4-[(4-fluorophenyl) (phenyl)methyl]-2,5-dimethylpiperazin-1-yl]-5-methyl-6-oxo-5,6-dihydro-1,5-naphthyridine-2-carbonitrile

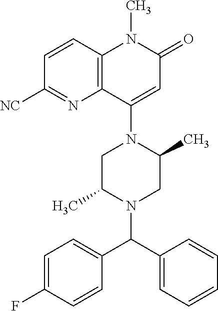 [SMILES: C[C@@H]1CN(c2cc(=O)n(C)c3ccc(C#N)nc23)[C@@H](C)CN1C(c1ccccc1)c1ccc(F)cc1]

In a 2 dram sealed reaction vessel, 8-((2S,5R)-2,5-dimethylpiperazin-1-yl)-5-methyl-6-oxo-5,6-dihydro-1,5-naphthyridine-2-carbonitrile, TFA (41.1 mg, 100 μmol), (4-fluorophenyl) (phenyl) methanol (28.3 mg, 140 μmol) and (cyanomethyl)trimethylphosphonium iodide (48.6 mg, 200 μmol) were combined in acetonitrile (200 μl). Hunig's Base (75 μL, 429 μmol) was added and the reaction mixture was heated at 110 °C for 2 hours. The reaction mixture was injected directly onto a 12 g silica gel column and eluted with 20-100% ethyl acetate in hexanes to afford Example 182 as a diasteromeric mixture. Analytical LC\MS conditions: Injection Vol=3 μL, Start % B 0, Final % B 100, Gradient Time 2 Minutes, Flow Rate 1 mL/min, Wavelength 220 nm, Solvent Pair acetonitrile/Water/TFA, Solvent A 10% acetonitrile, 90% water/0.05% TFA, Solvent B 10% Water, 90% acetonitrile/0.05% TFA, Column Acquity BEH C18 21. X 50 mm 1.7 μm, Oven Temp=40 °C. LC\MS results; retention time 1.4 minutes, observed mass 482.5 (M+).

The crude material was further purified via preparative LC/MS with the following conditions: Column: XBridge C18, 200 mm×19 mm, 5 μm particles; Mobile Phase A: 5:95 acetonitrile:water with 10 mM ammonium acetate; Mobile Phase B: 95:5 acetonitrile:water with 10 mM ammonium acetate; Gradient: a 0 minute hold at 47% B, 47-87% B over 20 minutes, then a 4 minute hold at 100% B; Flow Rate: 20 mL/min; Column Temperature: 25 °C. Fraction collection was triggered by MS signals. Fractions containing the product were combined and dried via centrifugal evaporation to afford 14.4 mg of the title compound (30% yield). Calculated molecular weight 481.575. LC\MS conditions QC-ACN-TFA-XB: Observed MS Ion 482.2, retention time 1.6 minutes. ¹H NMR (500 MHz, DMSO-d₆) δ 8.18-8.10 (m, 1H), 8.06 (d, J=8.8 Hz, 1H), 7.68-7.48 (m, 4H), 7.39-7.26 (m, 2H), 7.25-7.08 (m, 3H), 6.00 (s, 1H), 4.67 (br s, 1H), 4.59 (br d, J=6.7 Hz, 1H), 3.76-3.62 (m, 1H), 3.55 (br d, J=12.8 Hz, 1H), 3.15-3.04 (m, 1H), 2.90-2.81 (m, 1H), 2.36 (br dd, J=17.4, 11.9 Hz, 1H), 1.35-1.28 (m, 3H), 1.24 (s, 1H), 1.07 (br t, J=5.6 Hz, 3H).

### Method of Synthesizing DGKi Compounds 6 and 7

### 8-[(2S,5R)-4-[(4-fluorophenyl) (phenyl)methyl]-2,5-dimethylpiperazin-1-yl]-5-methyl-6-oxo-5,6-dihydro-1,5-naphthyridine-2-carbonitrile

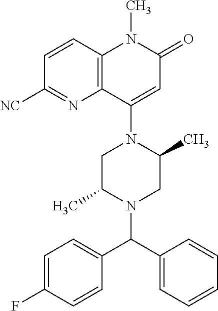 [SMILES: C[C@@H]1CN(c2cc(=O)n(C)c3ccc(C#N)nc23)[C@@H](C)CN1C(c1ccccc1)c1ccc(F)cc1]

Example 5 was separated into individual diastereomers using chiral solid phase chromatography: Column: Chiralpak OJ-H, 21×250 mm; 5 micron, Mobile Phase: 90% CO₂/10% methanol, Flow Conditions: 45 mL/min, Detector Wavelength: 225 nm, Injection Details: 500 μL, 15 mg dissolved in 1 mL methanol/acetonitrile.

The first eluting diastereomer, Example 6 (66.4 mg), was isolated in 20.2% yield. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50 °C; Gradient: 0% B to 100% B over 3 min, then a 0.50 min hold at 100% B; Flow: 1 mL/min; Detection: MS and UV (220 nm). Injection 1 results: Purity: 100.0%; Observed Mass: 482.1; Retention Time: 2.49 min. Injection 2 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50 °C; Gradient: 0% B to 100% B over 3 min, then a 0.50 min hold at 100% B; Flow: 1 mL/min; Detection: MS and UV (220 nm). Injection 2 results: Purity: 100.0%; Observed Mass: 482.11; Retention Time: 1.75 min.

The second eluting diastereomer, Example 7 (71.7 mg), was isolated in 21.9% yield. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50 °C; Gradient: 0% B to 100% B over 3 min, then a 0.50 min hold at 100% B; Flow: 1 mL/min; Detection: MS and UV (220 nm). Injection 1 results: Purity: 100.0%; Observed Mass: 482.11; Retention Time: 2.51 min. Injection 2 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50 °C; Gradient: 0% B to 100% B over 3 min, then a 0.50 min hold at 100% B; Flow: 1 mL/min; Detection: MS and UV (220 nm). Injection 2 results: Purity: 100.0%; Observed Mass: 482.1; Retention Time: 1.76 min.

### Method of Synthesizing DGKi Compound 8

### 4-[(2S,5R)-4-[(4-chlorophenyl)(4-fluorophenyl)methyl]-2,5-dimethylpiperazin-1-yl]-6-methoxy-1-methyl-1,2-dihydro-1,5-naphthyridin-2-one

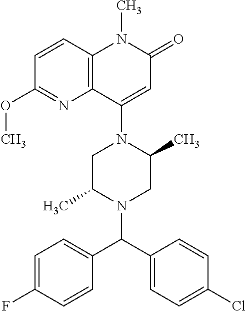 [SMILES: COc1ccc2c(n1)c(N1C[C@@H](C)N(C(c3ccc(F)cc3)c3ccc(Cl)cc3)C[C@@H]1C)cc(=O)n2C]

4-((2S,5R)-2,5-dimethylpiperazin-1-yl)-6-methoxy-1-methyl-1,5-naphthyridin-2(1H)-one (50 mg, 0.165 mmol) and 1-(bromo (4-chlorophenyl)methyl)-4-fluorobenzene (49.5 mg, 0.165 mmol) were combined with diisopropyl ethyl amine (0.173 mL, 0.992 mmol) in acetonitrile (3 mL) and the reaction mixture was heated at 55 °C overnight. LC/MS indicated the reaction was completed. The crude material was purified via preparative LC/MS with the following conditions: Column: XBridge C18, 200 mm×19 mm, 5 μm particles; Mobile Phase A: 5:95 acetonitrile:water with 10 mM ammonium acetate; Mobile Phase B: 95:5 acetonitrile:water with 10 mM ammonium acetate; Gradient: a 0 minute hold at 42% B, 42-82% B over 25 minutes, then a 5 minute hold at 100% B; Flow Rate: 20 mL/min; Column Temperature: 25 °C. Fraction collection was triggered by MS signals. Fractions containing the product were combined and dried via centrifugal evaporation. Calculated molecular weight 521.03. LC\MS conditions QC-ACN-AA-XB: Observed MS Ion 521.1, retention time 2.77 minutes.

### Method of Synthesizing DGKi Compound 9

### 8-[(2S,5R)-4-{[2-(difluoromethyl)-4-fluorophenyl]methyl}-2,5-dimethylpiperazin-1-yl]-5-methyl-6-oxo-5,6-dihydro-1,5-naphthyridine-2-carbonitrile

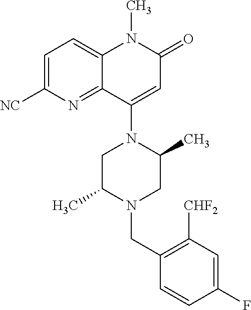 [SMILES: C[C@@H]1CN(c2cc(=O)n(C)c3ccc(C#N)nc23)[C@@H](C)CN1Cc1ccc(F)cc1C(F)F]

To a DMF (2 mL) solution of 8-((2S,5R)-2,5-dimethylpiperazin-1-yl)-5-methyl-6-oxo-5,6-dihydro-1,5-naphthyridine-2-carbonitrile (30 mg, 0.081 mmol) was added 2-(difluoromethyl)-4-fluorobenzaldehyde (16.86 mg, 0.097 mmol). The solution was stirred at room temperature for 1 hour. Sodium cyanoborohydride (15.22 mg, 0.242 mmol) was added and the reaction mixture was stirred at room temperature overnight. LC/MS analysis indicated the reaction was complete. The crude material was purified via preparative LC/MS with the following conditions: Column: XBridge C18, 200 mm×19 mm, 5 μm particles; Mobile Phase A: 5:95 acetonitrile:water with 10 mM ammonium acetate; Mobile Phase B: 95:5 acetonitrile:water with 10 mM ammonium acetate; Gradient: a 0 minute hold at 31% B, 31-71% B over 25 minutes, then a 5 minute hold at 100% B; Flow Rate: 20 mL/min; Column Temperature: 25 °C. Fraction collection was triggered by MS and UV signals. Fractions containing the product were combined and dried via centrifugal evaporation. The yield of the product was 13.0 mg, and the estimated purity by LCMS analysis was 100%. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50 °C; Gradient: 0% B to 100% B over 3 min, then a 0.50 min hold at 100% B; Flow: 1 mL/min; Detection: MS and UV (220 nm). Injection 1 results: Purity: 100.0%; Observed Mass: 456.08; Retention Time: 1.39 min. Injection 2 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50 °C; Gradient: 0% B to 100% B over 3 min, then a 0.50 min hold at 100% B; Flow: 1 mL/min; Detection: MS and UV (220 nm). Injection 2 results: Purity: 100.0%; Observed Mass: 456.07; Retention Time: 2.22 min. % B over 3 min, then a 0.50 min hold at 100% B; Flow: 1 mL/min; Detection: MS and UV (220 nm). Injection 2 results: Purity: 100.0%; Observed Mass: 456.07; Retention Time: 2.22 min.

### Method of Synthesizing DGKi Compound 10

### 8-[(2S,5R)-4-[(4-fluorophenyl)(4-methylphenyl)methyl]-2,5-dimethylpiperazin-1-yl]-5-methyl-6-oxo-5,6-dihydro-1,5-naphthyridine-2-carbonitrile

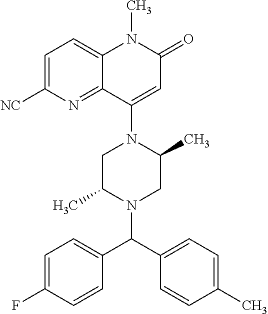 [SMILES: Cc1ccc(C(c2ccc(F)cc2)N2C[C@H](C)N(c3cc(=O)n(C)c4ccc(C#N)nc34)C[C@H]2C)cc1]

To the mixture of 8-((2S,5R)-2,5-dimethylpiperazin-1-yl)-5-methyl-6-oxo-5,6-dihydro-1,5-naphthyridine-2-carbonitrile, TFA (68.6 mg, 60% wt., 0.1 mmol), (cyanomethyl)trimethylphosphonium iodide (48.6 mg, 0.200 mmol) and (4-fluorophenyl) (p-tolyl) methanol (26.0 mg, 0.120 mmol) in acetonitrile (0.3 mL) was added Hunig's base (0.105 mL, 0.600 mmol). The reaction mixture was stirred at 110 °C for 2 hours, followed by a second addition of (cyanomethyl)trimethylphosphonium iodide (48.6 mg, 0.200 mmol), (4-fluorophenyl) (p-tolyl) methanol (26.0 mg, 0.120 mmol) and Hunig's base (0.058 mL, 0.300 mmol). The reaction mixture was stirred at 110 °C for another 2 hours. The crude reaction mixture was injected directly on 12 g Si-RediSep R_{f} for flash chromatography by 20-100% ethyl acetate in hexanes. Product containing fractions were combined and dried by vacuum. The resultant material was further purified via preparative LC/MS with the following conditions: Column: XBridge C18, 200 mm×19 mm, 5 μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0 minute hold at 20% B, 20-60% B over 25 minutes, then a 5 minute hold at 100% B; Flow Rate: 20 mL/min; Column Temperature: 25 °C Fraction collection was triggered by MS and UV signals. Fractions containing the product were combined and dried via centrifugal evaporation. The yield of the diastereomeric product TFA salt was 47.1 mg.

The diasteromeric product was resolved into two diastereomers by using SFC-chiral chromatography with the following conditions: Column: Chiral AD, 30×250 mm, 5 micron particles; Mobile Phase: 80% CO₂/20% IPA w/0.1% DEA; Flow Rate: 100 mL/min; Column Temperature: 25 °C. The title compound was collected as the 2^{nd} eluent peak, >91% de. Calculated molecular weight 495.602. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50 °C; Gradient: 0% B to 100% B over 3 min, then a 0.50 min hold at 100% B; Flow: 1 mL/min; Detection: MS and UV (220 nm). Injection 1 results: Purity: 97.6%; Observed Mass: 496.26; Retention Time: 2.52 min. Injection 2 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50 °C; Gradient: 0% B to 100% B over 3 min, then a 0.50 min hold at 100% B; Flow: 1 mL/min; Detection: MS and UV (220 nm). Injection 2 results: Purity: 98.2%; Observed Mass: 496.28; Retention Time: 1.73 min.

### Method of Synthesizing DGKi Compound 11

### 8-[(2S,5R)-4-[1-(2,6-difluorophenyl)ethyl]-2,5-dimethylpiperazin-1-yl]-5-methyl-6-oxo-5,6-dihydro-1,5-naphthyridine-2-carbonitrile

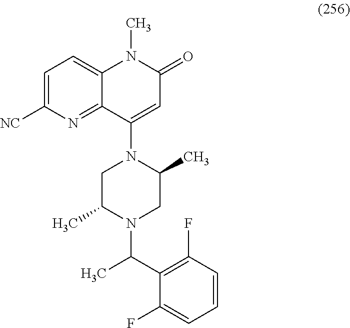 [SMILES: CC(c1c(F)cccc1F)N1C[C@H](C)N(c2cc(=O)n(C)c3ccc(C#N)nc23)C[C@H]1C]

To a mixture of 2-(1-bromoethyl)-1,3-difluorobenzene (15.12 mg, 0.065 mmol) and 5-methyl-6-oxo-8-(piperazin-1-yl)-5,6-dihydro-1,5-naphthyridine-2,7-dicarbonitrile, TFA (34.0 mg, 60% wt., 0.05 mmol) in acetonitrile (0.3 mL) was added Hunig's base (0.052 mL, 0.300 mmol). The mixture was stirred at 55 °C for 2 hours. LCMS indicated complete conversion to product. The crude material was purified via preparative LC/MS with the following conditions: Column: XBridge C18, 200 mm×19 mm, 5 μm particles; Mobile Phase A: 5:95 acetonitrile:water with 10 mM ammonium acetate; Mobile Phase B: 95:5 acetonitrile:water with 10 mM ammonium acetate; Gradient: a 0 minute hold at 37% B, 37-77% B over 20 minutes, then a 5 minute hold at 100% B; Flow Rate: 20 mL/min; Column Temperature: 25 °C. Fraction collection was triggered by MS and UV signals. Fractions containing the product were combined and dried via centrifugal evaporation. The yield of the product was 12.1 mg. Calculated molecular weight 437.495. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50 °C; Gradient: 0% B to 100% B over 3 min, then a 0.50 min hold at 100% B; Flow: 1 mL/min; Detection: MS and UV (220 nm). Injection 1 results: Purity: 100.0%; Observed Mass: 438.14; Retention Time: 2.36 min. Injection 2 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50 °C; Gradient: 0% B to 100% B over 3 min, then a 0.50 min hold at 100% B; Flow: 1 mL/min; Detection: MS and UV (220 nm). Injection 2 results: Purity: 100.0%; Observed Mass: 438.14; Retention Time: 1.2 min.

### Method of Synthesizing DGKi Compounds 12-14

### 8-((2S,5R)-4-(1-(2,4-difluorophenyl)propyl)-2,5-dimethylpiperazin-1-yl)-5-methyl-6-oxo-5,6-dihydro-1,5-naphthyridine-2-carbonitrile

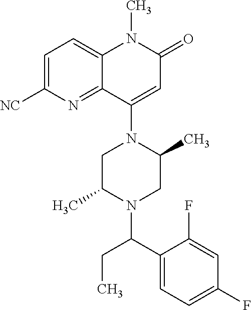 [SMILES: CCC(c1ccc(F)cc1F)N1C[C@H](C)N(c2cc(=O)n(C)c3ccc(C#N)nc23)C[C@H]1C]

To a mixture of 8-((2S,5R)-2,5-dimethylpiperazin-1-yl)-5-methyl-6-oxo-5,6-dihydro-1,5-naphthyridine-2-carbonitrile (29.7 mg, 0.1 mmol) and 1-(1-bromopropyl)-2,4-difluorobenzene (25.9 mg, 0.110 mmol) in acetonitrile (0.3 mL) was added Hunig's base (87 μL, 0.500 mmol). The mixture was stirred on hot plate at 55 °C for 16 hours. The crude material was purified via preparative LC/MS with the following conditions: Column: XBridge C18, 200 mm×19 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: a 0 minute hold at 3% B, 3-43% B over 25 minutes, then a 5 minute hold at 100% B; Flow Rate: 20 mL/min; Column Temperature: 25 °C. Fraction collection was triggered by MS and UV signals. Fractions containing the product were combined and dried via centrifugal evaporation. Stereochemistry: diasteromeric mixture.

The diastereomeric mixture of the synthesis of DGKi Compound 12 was further separated to resolve two homochiral diastereomers by using SFC-chiral chromatography with the following conditions: Column: Chiral OD, 30×250 mm, 5 micron particles; Mobile Phase: 15% IPA/85% CO₂ w/0.1% DEA; Flow Rate: 100 mL/min; Detector Wavelength: 220 nm.

DGKi Compound 13 (Isomer 1) was collected as the first eluent peak in 95% de. Stereochemistry: Homochiral.

DGKi Compound 14 (Isomer 2) was collected as the second eluent peak in 95% de. Stereochemistry: Homochiral.

### Method of Synthesizing DGKi Compound 15

### 8-(4-(bis(4-fluorophenyl)methyl)piperazin-1-yl)-5-methyl-7-nitro-6-oxo-5,6-dihydro-1,5-naphthyridine-2-carbonitrile

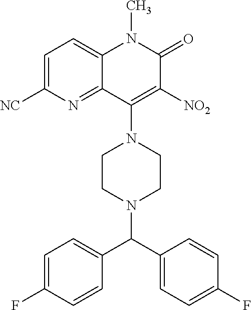 [SMILES: Cn1c(=O)c([N+](=O)[O-])c(N2CCN(C(c3ccc(F)cc3)c3ccc(F)cc3)CC2)c2nc(C#N)ccc21]

DMF was sparged with nitrogen for 1 hour. In a 1 dram vial was charged with zinc (0.95 mg, 0.015 mmol), bromo (tri-tert-butylphosphine) palladium (I) dimer (9.96 mg, 0.013 mmol) and 4-(4-(bis(4-fluorophenyl)methyl)piperazin-1-yl)-6-bromo-1-methyl-3-nitro-1,5-naphthyridin-2(1H)-one (21.38 mg, 0.037 mmol). The sparged DMF (0.3 mL) was added and the mixture was capped under nitrogen and immersed in a 50 °C oil bath for 15 minutes. Dicyanozinc (2.86 mg, 0.024 mmol) was added. The reaction mixture was capped under nitrogen and immersed in 50 °C oil bath for 3 hours. LC/MS analysis indicated the reaction was complete. The crude material was purified via preparative LC/MS with the following conditions: Column: XBridge C18, 19×200 mm, 5 μm particles; Mobile Phase A: 5:95 acetonitrile:water with 10 mM ammonium acetate; Mobile Phase B: 95:5 acetonitrile:water with 10 mM ammonium acetate; Gradient: 50-90% B over 15 minutes, then a 5 minute hold at 100% B; Flow: 20 mL/minute. Fractions containing the product were combined and dried via centrifugal evaporation. The title compound (11.4 mg) was isolated in 59.7% yield.

Alternative synthesis: A DMF (6 mL) solution of 8-chloro-5-methyl-7-nitro-6-oxo-5,6-dihydro-1,5-naphthyridine-2-carbonitrile (750 mg, 2.83 mmol) was combined with 1-(bis(4-fluorophenyl)methyl)piperazine (899 mg, 3.12 mmol)) followed by the addition of Hunig's Base (0.990 mL, 5.67 mmol). The reaction mixture was stirred at room temperature overnight. LC/MS analysis indicated the reaction was completed. The crude material was filtered and purified by preparative HPLC employing aqueous acetonitrile with ammonium acetate as the buffer to afford 1.02 g of yellow solid. Two analytical LC/MS injections were used to determine the final purity. Injection 1 conditions: Column: Waters Acquity UPLC BEH C18, 2.1×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50 °C; Gradient: 0-100% B over 3 minutes, then a 0.75 minute hold at 100% B; Flow: 1.0 mL/minute; Detection: UV at 220 nm. Injection 2 conditions: Column: Waters Acquity UPLC BEH C18, 2.1×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50 °C; Gradient: 0-100% B over 3 minutes, then a 0.75 minute hold at 100% B; Flow: 1.0 mL/minute; Detection: UV at 220 nm. Injection 1 results: Purity: 100%; Observed Mass: 517.0; Retention Time: 2.4 minutes. Injection 2 results: Purity: 98.4%; Observed Mass: 517.0; Retention Time: 1.7 minutes. ¹H NMR (500 MHz, chloroform-d) δ 7.88 (d, J=8.7 Hz, 1H), 7.76 (d, J=8.9 Hz, 1H), 7.40 (dd, J=8.5, 5.5 Hz, 4H), 7.02 (t, J=8.7 Hz, 4H), 4.34 (s, 1H), 3.68 (s, 3H), 3.62-3.55 (m, 4H), 2.64 (br s, 4H). ¹³C NMR (126 MHz, chloroform-d) δ 163.0, 161.0, 155.4, 147.0, 138.0, 137.7, 137.7, 135.9, 132.4, 129.5, 129.2, 129.2, 126.0, 123.1, 116.5, 115.8, 115.6, 74.3, 51.6, 51.2, 29.7.

### Method of Synthesizing DGKi Compound 16

### 8-[(2S,5R)-4-[bis(4-methylphenyl)methyl]-2,5-dimethylpiperazin-1-yl]-5-methyl-6-oxo-5,6-dihydro-1,5-naphthyridine-2-carbonitrile

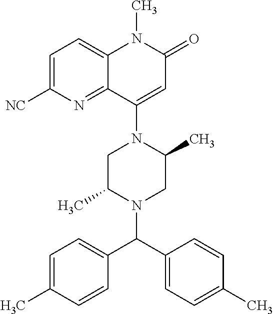

To the mixture of (cyanomethyl)trimethylphosphonium iodide (46.2 mg, 0.19 mmol), di-p-tolylmethanol (23.46 mg, 0.108 mmol), and 8-((2S,5R)-2,5-dimethylpiperazin-1-yl)-5-methyl-6-oxo-5,6-dihydro-1,5-naphthyridine-2-carbonitrile, TFA (72.4 mg, 54% wt, 0.095 mmol) in acetonitrile (0.3 mL) was added Hunig's base (0.10 mL, 0.57 mmol). The reaction mixture was stirred at 110 °C for 2 hours. The crude material was purified via preparative LC/MS with the following conditions: Column: XBridge C18, 200 mm×19 mm, 5 μm particles; Mobile Phase A: 5:95 acetonitrile:water with 10 mM ammonium acetate; Mobile Phase B: 95:5 acetonitrile:water with 10 mM ammonium acetate; Gradient: a 0 minute hold at 55% B, 55-95% B over 20 minutes, then a 4 minute hold at 100% B; Flow Rate: 20 mL/min; Column Temperature: 25 °C. Fraction collection was triggered by MS and UV signals. Fractions containing the product were combined and dried via centrifugal evaporation. The yield of the product was 23.4 mg. Calculated molecular weight 491.639. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50 °C; Gradient: 0% B to 100% B over 3 min, then a 0.75 min hold at 100% B; Flow: 1 mL/min; Detection: MS and UV (220 nm). Injection 1 results: Purity: 100.0%; Observed Mass: 492.21; Retention Time: 2.77 min. Injection 2 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50 °C; Gradient: 0% B to 100% B over 3 min, then a 0.75 min hold at 100% B; Flow: 1 mL/min; Detection: MS and UV (220 nm). Injection 2 results: Purity: 100.0%; Observed Mass: 492.2; Retention Time: 1.71 min. ¹H NMR (400 MHz, DMSO-d₆) δ ppm 8.15 (d, J=8.5 Hz, 1H), 8.04-8.09 (m, 1H), 7.81 (s, 4H), 7.57-7.63 (m, 2H), 7.12-7.19 (m, 2H), 6.00 (s, 1H), 4.82 (s, 1H), 4.52-4.63 (m, 1H), 3.64-3.76 (m, 1H), 3.51-3.58 (m, 4H), 2.99-3.10 (m, 1H), 2.86 (br d, J=8.5 Hz, 1H), 2.28-2.37 (m, 1H), 1.31 (d, J=6.5 Hz, 3H), 1.07 (d, J=6.5 Hz, 3H). ¹³C NMR (100.66 MHz, DMSO-d₆) δ ppm 162.4, 160.9, 159.9, 153.5, 148.0, 138.7, 138.6, 135.0, 132.6, 129.3 (d, J=8.0 Hz), 128.8 (d, J=10.0 Hz), 124.0, 122.8, 118.6, 117.5, 115.6, 115.4, 109.8, 104.8, 69.0, 51.8, 49.4, 48.9, 47.2, 28.6, 13.4, 7.4.

### DGKi Compounds 17 and 18

### 4-((2S,5R)-2,5-Diethyl-4-(1-(4-(trifluoromethyl)phenyl)propyl)piperazin-1-yl)-1-methyl-2-oxo-1,2-dihydropyrido[3,2-d]pyrimidine-6-carbonitrile

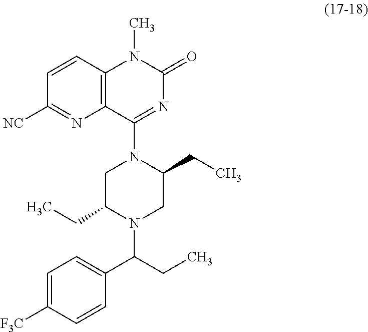 [SMILES: CCC(c1ccc(C(F)(F)F)cc1)N1C[C@H](CC)N(c2nc(=O)n(C)c3ccc(C#N)nc23)C[C@H]1CC]

To a stirred solution of 4-((2S,5R)-2,5-diethylpiperazin-1-yl)-1-methyl-2-oxo-1,2-dihydropyrido[3,2-d]pyrimidine-6-carbonitrile, TFA (0.12 g, 0.27 mmol) in acetonitrile (10 mL) were added DIPEA (0.14 mL, 0.82 mmol), 1-(1-chloropropyl)-4-(trifluoromethyl)benzene (0.12 g, 0.55 mmol), and sodium iodide (0.04 g, 0.27 mmol). The reaction mixture was heated at 85 °C for 16 h. The reaction mixture was cooled to room temperature and the solvent was removed under reduced pressure to yield the crude product, which was purified by preparative HPLC [HPLC Method: Column: Sunfire C18, 150×19 mm ID, 5 μm; Mobile Phase A: 10 mM ammonium acetate in water; Mobile Phase B: acetonitrile; Gradient: 0-100% B over 18 minutes, then a 5 minute hold at 100% B; Flow: 17 mL/min]. The fractions were concentrated under reduced pressure and lyophilized from EtOH/H₂O (1:5) to yield Compounds 17 and 18.

Compound 17: (10 mg, 7% yield); LCMS: m/z=513.3 (M+H); rt 2.52 min; (LCMS method: Column: XBridge BEH XP C18 (50×2.1) mm, 2.5 μm Mobile phase A: 95% water: 5% acetonitrile; 10 mM ammonium formate; Mobile phase B: 5% Water: 95% acetonitrile; 10 mM ammonium formate; Flow: 1.1 mL/min; Temp: 50 °C; Time (min): 0-4; % B: 0-100; 1H NMR (400 MHz, DMSO-d₆) δ 8.24 (d, J=6.6 Hz, 1H), 7.98 (d, J=9.0 Hz, 1H), 7.73 (d, J=8.1 Hz, 2H), 7.56 (d, J=7.1 Hz, 2H), 5.83-5.48 (m, 1H), 4.98-4.86 (m, 1H), 3.64 (br. s., 1H), 3.43 (s, 3H), 3.08 (d, J=9.8 Hz, 1H), 2.93-2.82 (m, 2H), 2.42-2.26 (m, 1H), 2.13-2.08 (m, 1H), 1.98-1.82 (m, 3H), 1.66-1.54 (m, 1H), 1.44-1.31 (m, 1H), 0.98-0.91 (br. s., 3H), 0.69-0.53 (m, 6H).

Compound 18: (3 mg, 2% yield); LCMS: m/z=513.3 (M+H); rt 2.54 min; (LCMS method: Column: XBridge BEH XP C18 (50×2.1) mm, 2.5 μm Mobile phase A: 95% water: 5% acetonitrile; 10 mM ammonium formate; Mobile phase B: 5% Water: 95% acetonitrile; 10 mM ammonium formate; Flow: 1.1 mL/min; Temp: 50 °C; Time (min): 0-4; % B: 0-100; 1H NMR (400 MHz, DMSO-d₆) δ 8.28-8.19 (m, 1H), 8.01-7.95 (m, 1H), 7.72 (d, J=7.8 Hz, 2H), 7.58 (d, J=8.6 Hz, 2H), 6.06-5.28 (m, 1H), 5.08-4.76 (m, 1H), 3.64-3.50 (m, 2H), 3.43 (s, 3H), 3.16-3.08 (m, 1H), 2.25-2.14 (m, 2H), 2.00-1.83 (m, 3H), 1.57-1.53 (m, 3H), 1.03-0.89 (m, 3H), 0.65-0.54 (m, 6H).

### DGKi Compounds 19 and 20

### 4-((2S,5R)-5-Ethyl-2-methyl-4-(1-(4-(trifluoromethyl)phenyl)ethyl)piperazin-1-yl)-1-methyl-2-oxo-1,2-dihydropyrido[3,2-d]pyrimidine-6-carbonitrile

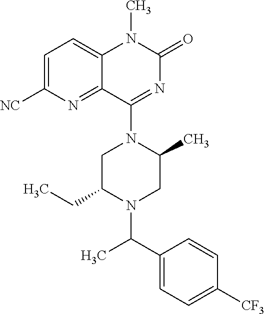 [SMILES: CC[C@@H]1CN(c2nc(=O)n(C)c3ccc(C#N)nc23)[C@@H](C)CN1C(C)c1ccc(C(F)(F)F)cc1]

To a stirred solution of 4-((2S,5R)-5-ethyl-2-methylpiperazin-1-yl)-1-methyl-2-oxo-1,2-dihydropyrido[3,2-d]pyrimidine-6-carbonitrile, TFA (70 mg, 0.22 mmol) in acetonitrile (2 mL) at room temperature were added DIPEA (0.12 mL, 0.67 mmol), 1-(1-chloroethyl)-4-(trifluoromethyl)benzene (93 mg, 0.45 mmol), sodium iodide (33.6 mg, 0.22 mmol) and heated at 85 °C for 16 h. The reaction mixture cooled to room temperature and the solvent was removed under reduced pressure, the residue was dissolved in ethyl acetate (100 mL). The organic layer was washed with brine, dried over Na₂SO₄ and concentrated under reduced pressure to yield the crude product, which was purified by preparative HPLC [HPLC Method: Column: Sunfire C18 (150 mm×19.2 mm ID, 5 μm), Mobile phase A=10 mM ammonium acetate in water, Mobile phase B=acetonitrile, Flow: 19 mL/min], fractions were concentrated under reduced pressure, diluted with EtOH/H₂O (1:5), and lyophilized to yield Compounds 19 and 20.

Compound 19: (9 mg, 8% yield); LCMS: m/z=485.1 (M+H); rt 2.34 min; (LCMS method: Column: XBridge BEH XP C18 (50×2.1 mm), 2.5 μm; Mobile phase A: 95% water: 5% acetonitrile; 10 mM ammonium acetate; Mobile phase B: 5% Water: 95% acetonitrile; 10 mM ammonium acetate; Flow: 1.1 mL/min; Temp: 50 °C; Time (min): 0-3; % B: 0-100. ¹H NMR (400 MHz, DMSO-d₆) δ ppm 8.32-8.17 (m, 1H), 8.05-7.94 (m, 1H), 7.76-7.66 (m, 2H), 7.66-7.55 (m, 2H), 6.11-5.42 (m, 1H), 5.10-4.79 (m, 1H), 3.78-3.59 (m, 2H), 3.44 (s, 3H), 3.17-3.05 (m, 1H), 2.64-2.55 (m, 1H), 2.26-2.09 (m, 1H), 1.65-1.34 (m, 3H), 1.31-1.16 (m, 5H), 1.01 (br t, J=7.1 Hz, 3H).

Compound 20: (9 mg, 8% yield); LCMS: m/z=485.1 (M+H); rt 2.29 min; (LCMS Method: Column: XBridge BEH XP C18 (50×2.1 mm), 2.5 μm; Mobile phase A: 95% water: 5% acetonitrile; 10 mM ammonium acetate; Mobile phase B: 5% Water: 95% acetonitrile; 10 mM ammonium acetate; Flow: 1.1 mL/min; Temp: 50 °C; Time (min): 0-3; % B: 0-100%). ¹H NMR (400 MHz, DMSO-d₆) δ ppm 8.24 (br d, J=8.6 Hz, 1H), 7.99 (d, J=9.0 Hz, 1H), 7.73 (d, J=8.3 Hz, 2H), 7.61 (br d, J=8.3 Hz, 2H), 5.87-5.63 (m, 1H), 5.10-4.79 (m, 1H), 3.90-3.80 (m, 1H), 3.44 (s, 3H), 3.46-3.15 (m, 1H), 2.89-2.73 (m, 2H), 2.41-2.34 (m, 1H), 1.63-1.34 (m, 5H), 1.29 (br d, J=6.1 Hz, 3H), 0.79-0.64 (m, 3H).

### DGKi Compounds 21 and 22

### 4-((2S,5R)-5-Ethyl-4-((4-fluorophenyl)(5-(trifluoromethyl)pyridin-2-yl)methyl)-2-methylpiperazin-1-yl)-1-methyl-2-oxo-1,2-dihydropyrido[3,2-d]pyrimidine-6-carbonitrile

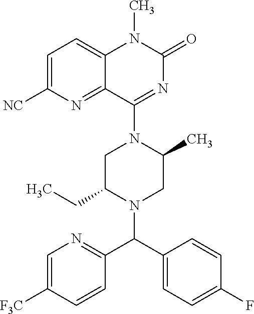 [SMILES: CC[C@@H]1CN(c2nc(=O)n(C)c3ccc(C#N)nc23)[C@@H](C)CN1C(c1ccc(F)cc1)c1ccc(C(F)(F)F)cn1]

To a stirred solution of 4-((2S,5R)-5-ethyl-2-methylpiperazin-1-yl)-1-methyl-2-oxo-1,2-dihydropyrido[3,2-d]pyrimidine-6-carbonitrile, TFA (0.5 g, 1.17 mmol) in acetonitrile (10 mL) was added DIPEA (1.02 mL, 5.86 mmol), followed by 2-(bromo (4-fluorophenyl)methyl)-5-(trifluoromethyl)pyridine (0.78 mg, 2.35 mmol). The reaction mixture was heated at 80 °C for 3 h. The reaction mixture was cooled to room temperature and the solvent was removed under reduced pressure to yield the crude product, which was purified by preparative HPLC(HPLC Method: Column: INERTSIL ODS 21.2×250 mm, 5 μm; Mobile Phase A: 0.1% TFA in water; Mobile Phase B: acetonitrile; Gradient: 30-80% B over 14 minutes, then a 5 minute hold at 100% B; Flow: 17 mL/min), fractions were concentrated under reduced pressure and lyophilized from (EtOH/H₂O, 1:5) to yield Compound 21 and Compound 22.

Compound 21:140 mg, 21% yield; LCMS: m/z=566.2 (M+H); rt 3.26 min; (LCMS method: Column: Column-Kinetex XB-C18 (75×3 mm-2.6 μm), Mobile phase A: 98% water: 2% acetonitrile; 10 mM ammonium formate; Mobile phase B: 2% Water: 98% acetonitrile; 10 mM ammonium formate; Flow: 1.0 mL/min; Temp: 50 °C; Time (min): 0-4; % B: 0-100%). ¹H NMR (400 MHz, DMSO-d₆) δ ppm 8.83 (br s, 1H), 8.19-8.31 (m, 2H), 7.95-8.12 (m, 2H), 7.53-7.63 (m, 2H), 7.12-7.26 (m, 2H), 5.41-6.26 (m, 1H), 4.79-5.20 (m, 2H), 3.60-3.74 (m, 1H), 3.44 (s, 3H), 2.73-2.87 (m, 1H), 2.22-2.42 (m, 2H), 1.40-1.68 (m, 5H), 0.53-0.71 (m, 3H).

Compound 22:155 mg, 23% yield; LCMS: m/z=566.2 (M+H); rt 3.25 min; (LCMS method: Column: Column-Kinetex XB-C18 (75×3 mm-2.6 μm), Mobile phase A: 98% water: 2% acetonitrile; 10 mM ammonium formate; Mobile phase B: 2% Water: 98% acetonitrile; 10 mM ammonium formate; Flow: 1.0 mL/min; Temp: 50 °C; Time (min): 0-4; % B: 0-100%). ¹H NMR (400 MHz, DMSO-d₆) δ ppm 8.92 (s, 1H), 8.17-8.27 (m, 2H), 7.90-8.02 (m, 2H), 7.60-7.67 (m, 2H), 7.14-7.22 (m, 2H), 5.52-6.07 (m, 1H), 4.87-5.08 (m, 2H), 3.39-3.71 (m, 4H), 2.69-2.78 (m, 1H), 2.37-2.45 (m, 1H), 1.37-1.69 (m, 5H), 0.58-0.77 (m, 3H).

### DGKi Compounds 23-24

### (4-((2S,5R)-4-((4-chlorophenyl) (pyridin-2-yl)methyl)-5-ethyl-2-methylpiperazin-1-yl)-1-methyl-2-oxo-1,2-dihydropyrido[3,2-d]pyrimidine-6-carbonitrile

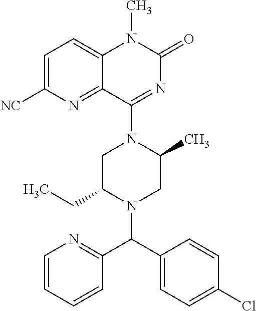 [SMILES: CC[C@@H]1CN(c2nc(=O)n(C)c3ccc(C#N)nc23)[C@@H](C)CN1C(c1ccc(Cl)cc1)c1ccccn1]

To a stirred solution of 4-((2S,5R)-5-ethyl-2-methylpiperazin-1-yl)-1-methyl-2-oxo-1,2-dihydropyrido[3,2-d]pyrimidine-6-carbonitrile (100 mg, 0.32 mmol) in acetonitrile (5 mL) was added DIPEA (0.3 mL, 1.60 mmol), followed by 2-(bromo (4-chlorophenyl)methyl)pyridine (181 mg, 0.64 mmol). The reaction mixture was heated at 80 °C for 3 h. The reaction mixture was cooled to room temperature and the solvent was removed under reduced pressure to yield the crude product, which was purified by preparative HPLC(HPLC Method: Column: Cellulose-5(250*20 ID) 5 micron; Mobile Phase A: 0.1% DEA in IPA; Mobile Phase B: 0.1% DEA in ACN; Gradient: 90% of B, then a 5 minute hold at 100% B; Flow: 18 mL/min), fractions were concentrated under reduced pressure and lyophilized from (EtOH/H₂O, 1:5) to yield Compound 23 and Compound 24.

Compound 23:24 mg, 14% yield; LCMS: m/z=514.2 (M+H); rt 2.94 min; (LCMS method: Column: Column-Kinetex XB-C18 (75×3 mm-2.6 μm), Mobile phase A: 98% water: 2% acetonitrile; 10 mM ammonium formate; Mobile phase B: 2% Water: 98% acetonitrile; 10 mM ammonium formate; Flow: 1.0 mL/min; Temp: 50 °C; Time (min): 0-4; % B: 0-100%). ¹H NMR (400 MHz, DMSO-d₆): δ ppm 8.52 (d, J=4.5 Hz, 1H), 8.23 (d, J=9.0 Hz, 1H), 7.96-8.02 (m, 1H), 7.75-7.81 (m, 1H), 7.59-7.68 (m, 3H), 7.39 (d, J=8.5 Hz, 2H), 7.22-7.29 (m, 1H), 5.54-5.95 (m, 1H), 4.81-5.07 (m, 2H), 3.39-3.68 (m, 5H), 2.69-2.76 (m, 1H), 2.35-2.44 (m, 1H), 1.37-1.67 (m, 5H), 0.58-0.67 (m, 3H).

Compound 24:22 mg, 13% yield; LCMS: m/z=514.2 (M+H); rt 2.94 min; (LCMS method: Column: Column-Kinetex XB-C18 (75×3 mm-2.6 μm), Mobile phase A: 98% water: 2% acetonitrile; 10 mM ammonium formate; Mobile phase B: 2% Water: 98% acetonitrile; 10 mM ammonium formate; Flow: 1.0 mL/min; Temp: 50 °C; Time (min): 0-4; % B: 0-100%). ¹H NMR (400 MHz, DMSO-d₆): δ ppm 8.41-8.45 (m, 1H), 8.23 (d, J=9.0 Hz, 1H), 7.96-8.02 (m, 1H), 7.78-7.85 (m, 2H), 7.53-7.61 (m, 2H), 7.40 (d, J=8.5 Hz, 2H), 7.20-7.26 (m, 1H), 5.52-5.97 (m, 1H), 4.87-5.04 (m, 1H), 4.78-4.86 (m, 1H), 3.37-3.71 (m, 4H), 2.72-2.78 (m, 1H), 2.54-2.63 (m, 1H), 2.35-2.46 (m, 1H), 1.40-1.64 (m, 5H), 0.58-0.70 (m, 3H).

### DGKi Compounds 25 and 26

### 4-((2S,5R)-4-((3-Cyclopropyl-1,2,4-oxadiazol-5-yl)(4-fluorophenyl)methyl)-2,5-dimethylpiperazin-1-yl)-1-methyl-2-oxo-1,2-dihydropyrido[3,2-d]pyrimidine-6-carbonitrile

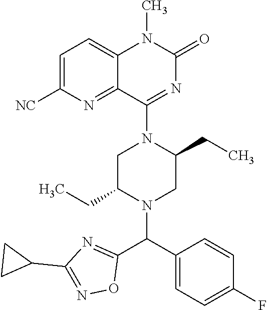 [SMILES: CC[C@H]1CN(C(c2ccc(F)cc2)c2nc(C3CC3)no2)[C@H](CC)CN1c1nc(=O)n(C)c2ccc(C#N)nc12]

To a stirred solution of 2-((2R,5S)-4-(6-cyano-1-methyl-2-oxo-1,2-dihydropyrido[3,2-d]pyrimidin-4-yl)-2,5-diethylpiperazin-1-yl)-2-(4-fluorophenyl) acetic acid, (0.045 g, 0.09 mmol), N-hydroxycyclopropanecarboximidamide (9.4 mg, 0.09 mmol) in DMF (2 mL), BOP (0.01 g, 0.23 mmol) and triethylamine (0.04 mL, 0.23 mmol) were added at room temperature. After 2 hours, the reaction mixture was heated at 110 °C for 3 h. The reaction mixture was cooled to room temperature and evaporated under reduced pressure to yield crude product, which was purified via preparative HPLC. Chiral Separation Method: Column: DAD-1-Cellulose-2(250×4.6 mm), 5 micron. Mobile Phase: 0.1% DEA in acetonitrile, Flow: 2.0 mL\min.

Compound 25: (1.9 mg, 6% yield): LCMS: m/z, 543.3 (M+H); rt 2.21 min; LCMS method: Column: XBridge BEH XP C18 (50×2.1) mm, 2.5 μm; Mobile phase A: 95% water: 5% acetonitrile; 10 mM ammonium acetate; Mobile phase B: 5% water: 95% acetonitrile; 10 mM ammonium acetate; Flow: 1.1 mL/min; Temp: 50 °C; Time (min) Time (min): 0-3; % B: 0-100%). ¹H NMR (400 MHz, DMSO-d₆) δ ppm 8.29-8.16 (m, 1H), 8.06-7.92 (m, 1H), 7.75-7.58 (m, 2H), 7.26 (m, 2H), 6.01-5.32 (m, 1H), 5.28 (br s, 1H), 5.00-4.79 (m, 1H), 3.66-3.56 (m, 1H), 3.43 (s, 3H), 2.65-2.57 (m, 1H), 2.44-2.34 (m, 2H), 2.18-2.00 (m, 1H), 1.95-1.74 (m, 2H), 1.68-1.34 (m, 2H), 1.15-1.02 (m, 2H), 0.93-0.83 (m, 2H), 0.81-0.62 (m, 6H).

Compound 26: (1.0 mg, 3% yield): LCMS: m/z, 543.3 (M+H); rt 2.20 min; LCMS method: Column: XBridge BEH XP C18 (50×2.1) mm, 2.5 μm; Mobile phase A: 95% water: 5% acetonitrile; 10 mM ammonium acetate; Mobile phase B: 5% water: 95% acetonitrile; 10 mM ammonium acetate; Flow: 1.1 mL/min; Temp: 50 °C; Time (min) Time (min): 0-3; % B: 0-100%). ¹H NMR (400 MHz, DMSO-d₆) δ ppm 8.23 (d, J=8.8 Hz, 1H), 8.06-7.91 (m, 1H), 7.62 (dd, J=6.2, 7.5 Hz, 2H), 7.26 (t, J=8.8 Hz, 2H), 5.92-5.31 (m, 1H), 5.29 (s, 1H), 4.96-4.78 (m, 1H), 3.60-3.50 (m, 1H), 3.43 (s, 3H), 3.25-3.10 (m, 1H), 2.97-2.75 (m, 2H), 2.27-1.65 (m, 3H), 1.49-1.24 (m, 2H), 1.11-0.97 (m, 2H), 0.94-0.75 (m, 5H), 0.74-0.50 (m, 3H).

### DGKi Compounds 27 and 28

### 4-((2S,5R)-4-((4-fluorophenyl)(5-(trifluoromethyl)pyridin-2-yl)methyl)-2,5-dimethylpiperazin-1-yl)-1-methyl-2-oxo-1,2-dihydropyrido[3,2-d]pyrimidine-6-carbonitrile

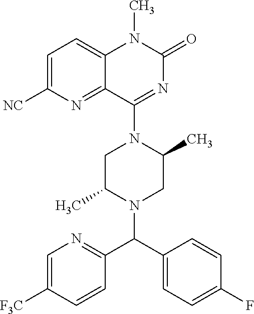 [SMILES: C[C@@H]1CN(c2nc(=O)n(C)c3ccc(C#N)nc23)[C@@H](C)CN1C(c1ccc(F)cc1)c1ccc(C(F)(F)F)cn1]

To a stirred solution of 4-((2S,5R)-2,5-dimethylpiperazin-1-yl)-1-methyl-2-oxo-1,2-dihydropyrido[3,2-d]pyrimidine-6-carbonitrile (1 g, 3.35 mmol) in acetonitrile (10 mL) was added DIPEA (5.9 mL, 33.5 mmol), followed by 2-(bromo (4-fluorophenyl)methyl)-5-(trifluoromethyl)pyridine (2.24 g, 6.70 mmol). The reaction mixture was heated at 80 °C for 4 h. The reaction mixture was cooled to room temperature and the solvent was removed under reduced pressure to yield the crude product, which was purified by preparative HPLC(HPLC Method: Column: Sunfire C18, 150×19 mm ID, 5 μm; Mobile Phase A: 0.1% TFA in water; Mobile Phase B: Acetonitrile:MeOH (1:1); Gradient: 50-100% B over 20 minutes, then a 5 minute hold at 100% B; Flow: 19 mL/min), fractions were concentrated under reduced pressure and lyophilized from (EtOH/H₂O, 1:5) to yield Compound 27 and Compound 28.

Compound 27:110 mg, 6% yield; LCMS: m/z=552.2 (M+H); rt 3.09 min; (LCMS method: Column: Column-Kinetex XB-C18 (75×3 mm-2.6 μm), Mobile phase A: 98% water: 2% acetonitrile; 10 mM ammonium formate; Mobile phase B: 2% Water: 98% acetonitrile; 10 mM ammonium formate; Flow: 1.0 mL/min; Temp: 50 °C; Time (min): 0-4; % B: 20-100%). ¹H NMR (400 MHz, DMSO-d₆) δ ppm 8.83 (s, 1H), 8.22 (d, J=9.0 Hz, 2H), 8.11-7.95 (m, 2H), 7.71-7.58 (m, 2H), 7.25-7.13 (m, 2H), 5.76-5.44 (m, 1H), 5.13-4.67 (m, 2H), 3.86-3.49 (m, 1H), 3.44 (s, 3H), 3.19-3.08 (m, 1H), 2.84 (dd, J=3.8, 12.3 Hz, 1H), 2.38-2.26 (m, 1H), 1.67-1.39 (m, 3H), 1.11-0.86 (m, 3H).

Compound 28:145 mg, 8% yield; LCMS: m/z=552.2 (M+H); rt 3.09 min; (LCMS method: Column: Column-Kinetex XB-C18 (75×3 mm-2.6 μm), Mobile phase A: 98% water: 2% acetonitrile; 10 mM ammonium formate; Mobile phase B: 2% Water: 98% acetonitrile; 10 mM ammonium formate; Flow: 1.0 mL/min; Temp: 50 °C; Time (min): 0-4; % B: 0-100%). ¹H NMR (400 MHz, DMSO-d₆) δ ppm 8.91 (s, 1H), 8.27-8.16 (m, 2H), 7.99 (d, J=9.0 Hz, 2H), 7.69-7.57 (m, 2H), 7.23-7.13 (m, 2H), 5.77-5.41 (m, 1H), 5.09-4.62 (m, 2H), 3.90-3.65 (m, 1H), 3.44 (s, 3H), 3.14-3.02 (m, 1H), 2.80-2.74 (m, 1H), 1.61-1.40 (m, 3H), 1.10-0.93 (m, 3H) [1H obscured with solvent peak].

### DGKi Compounds 29 and 30

### 4-((2S,5R)-4-(1-(4-(cyclopropylmethoxy)-2-fluorophenyl)propyl)-2,5-diethylpiperazin-1-yl)-1-methyl-2-oxo-1,2-dihydropyrido[3,2-d]pyrimidine-6-carbonitrile

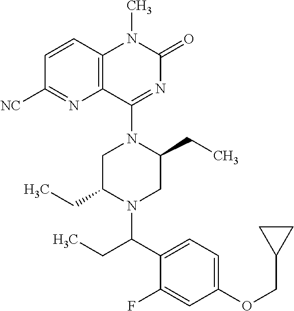 [SMILES: CCC(c1ccc(OCC2CC2)cc1F)N1C[C@H](CC)N(c2nc(=O)n(C)c3ccc(C#N)nc23)C[C@H]1CC]

To a stirred solution of 4-((2S,5R)-2,5-diethylpiperazin-1-yl)-1-methyl-2-oxo-1,2-dihydropyrido[3,2-d]pyrimidine-6-carbonitrile, HCl (200 mg, 0.55 mmol) in acetonitrile (5 mL) were added DIPEA (0.3 mL, 1.65 mmol), sodium iodide (83 mg, 0.55 mmol) and 1-(1-chloropropyl)-4-(cyclopropylmethoxy)-2-fluorobenzene (268 mg, 1.1 mmol). The reaction mixture was heated at 80 °C for 16 h. The reaction mixture was allowed to cool to room temperature. Another lot of 1-(1-chloropropyl)-4-(cyclopropylmethoxy)-2-fluorobenzene (268 mg, 1.102 mmol) was added and continued heating for another 16 h. The reaction mixture was cooled, the solvent was removed under reduced pressure and the residue was dissolved in ethyl acetate (10×20 mL). The organic layer was washed with brine, dried over Na₂SO₄, concentrated under reduced pressure to yield the crude product which was purified by preparative HPLC. HPLC method: Column: EXRS (20×250 mm, 5 μm), mobile phase A-10 mM ammonium acetate in water R, mobile phase A-B: acetonitrile, FLOW: 20 mL/min.

Fraction 1 was concentrated under reduced pressure and the product was diluted with (EtOH/H₂O, 1:5) and lyophilized to yield Compound 29(35 mg, 11.6% yield); LCMS: m/z, 533.4 [M+H]⁺, rt 1.57 min; (LCMS method: Column: KINETIX XB C18 (75×3 mm, 2.6 μm); mobile phase A: 10 mM ammonium acetate in water (pH 3.3), mobile phase B: acetonitrile. ¹H NMR (DMSO-d₆, 400 MHz) δ (ppm) 8.23 (d, J=9.0 Hz, 1H), 7.97 (d, J=9.0 Hz, 1H), 7.33 (m, 1H), 6.62-6.92 (m, 2H), 5.29-6.06 (m, 1H), 4.70-5.05 (m, 1H), 3.82 (m, 3H), 3.43 (s, 3H), 2.99-3.10 (m, 1H), 2.80-2.87 (m, 1H), 2.63-2.78 (m, 1H), 2.33 (s, 1H), 1.74-2.11 (m, 3H), 1.51-1.66 (m, 1H), 1.17-1.46 (m, 3H), 0.84-1.01 (m, 3H), 0.61-0.78 (m, 6H), 0.53-0.61 (m, 2H), 0.29-0.35 (m, 2H).

Fraction 2 was concentrated under reduced pressure and the product was diluted with (EtOH/H₂O, 1:5) and lyophilized to yield Compound 30 (37 mg, 12.35% yield); LCMS: m/z, 533.4 [M+H]⁺, rt 2.72 min; [(LCMS Method: Column: KINETIX XB C18 (75×3 mm, 2.6 μm); mobile phase A: 10 mM ammonium acetate in water (pH 3.3), mobile phase B: acetonitrile. ¹H NMR (DMSO-d₆, 400 MHz): δ (ppm) 8.13-8.35 (m, 1H), 7.98 (m, 1H), 7.38 (m, 1H), 6.61-6.89 (m, 2H), 5.18-6.15 (m, 1H), 4.66-5.13 (m, 1H), 3.63-3.90 (m, 3H), 3.43 (s, 3H), 3.25 (m, 1H), 3.00-3.15 (m, 1H), 2.63-2.70 (m, 1H), 2.26-2.38 (m, 1H), 1.81 (m, 3H), 1.35-1.61 (m, 2H), 1.15-1.26 (m, 2H), 0.88-1.00 (m, 3H), 0.61-0.71 (m, 6H), 0.51-0.59 (m, 2H), 0.32 (m, 2H).

### DGKi Compounds 31 and 32

### 4-((2S,5R)-2,5-Diethyl-4-(1-(4-(trifluoromethyl)phenyl)butyl)piperazin-1-yl)-1-methyl-2-oxo-1,2-dihydropyrido[3,2-d]pyrimidine-6-carbonitrile

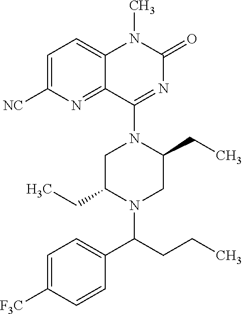 [SMILES: CCCC(c1ccc(C(F)(F)F)cc1)N1C[C@H](CC)N(c2nc(=O)n(C)c3ccc(C#N)nc23)C[C@H]1CC]

To a stirred solution of 4-((2S,5R)-2,5-diethylpiperazin-1-yl)-1-methyl-2-oxo-1,2-dihydropyrido[3,2-d]pyrimidine-6-carbonitrile, HCl (0.4 g, 1.1 mmol) in acetonitrile (10 mL) was added DIPEA (0.6 mL, 3.31 mmol), followed by 1-(1-chlorobutyl)-4-trifluoromethyl)benzene (0.783 g, 3.31 mmol) and sodium iodide (0.165 g, 1.102 mmol). The reaction mixture was heated at 85 °C for 16 h. The reaction mixture was filtered through a Celite pad, washed with ethyl acetate and the filtrate was concentrated under reduced pressure to give the crude compound, which was purified by preparative HPLC [HPLC Method: Column: YMC ExRS (250 mm×21.2 mm, 5 μm) Mobile phase A=10 mM ammonium acetate pH 4.5 in water. Mobile phase B=acetonitrile Gradient: 80% B over 2 minutes, then a 16 minute hold at 100% B; Flow: 19 mL/min) to yield Compounds 31 and 32.

Compound 31: (10 mg, 1.7% yield), LCMS: m/z=527.4 (M+H); rt 2.626 min; [LCMS Method: Column: XBridge BEH XP C18 (50×2.1 mm), 2.5 μm; Mobile phase A: 95% Water: 5% Acetonitrile; 10 mM NH₄OAC; Mobile phase B: 5% Water: 95% Acetonitrile; 10 mM NH₄OAC; Flow: 1.1 mL/min; Temp: 50 °C; Time (min)]. ¹H NMR (400 MHz, DMSO-d₆) δ 8.30-8.16 (m, 1H), 7.98 (d, J=9.0 Hz, 1H), 7.72 (d, J=8.3 Hz, 2H), 7.56 (br d, J=7.8 Hz, 2H), 5.86-5.44 (m, 1H), 5.01-4.77 (m, 1H), 3.730-3.718 (m, 1H), 3.46 (s, 3H), 3.43-3.35 (m, 1H) 3.13-3.01 (m, 1H), 2.93-2.75 (m, 2H), 2.38-2.26 (m, 1H), 2.17-1.74 (m, 3H), 1.63-1.22 (m, 3H), 1.01-0.86 (m, 4H), 0.84-0.75 (m, 3H), 0.73-0.54 (m, 3H).

Compound 32: (7.2 mg, 1.23% yield), LCMS: m/z=527.3 (M+H); rt 2.654 min; [LCMS Method: Column: XBridge BEH XP C18 (50×2.1) mm, 2.5 μm; Mobile phase A: 95% Water: 5% Acetonitrile; 10 mM NH₄OAC; Mobile phase B: 5% Water: 95% Acetonitrile; 10 mM NH₄OAC; Flow: 1.1 mL/min; Temp: 50 °C; Time (min)]. ¹H NMR (400 MHz, DMSO-d₆) δ=8.29-8.15 (m, 1H), 7.96-8.02 (m, 1H), 7.70 (d, J=8.1 Hz, 2H), 7.58 (br d, J=8.1 Hz, 2H), 6.09-5.22 (m, 1H), 5.13-4.66 (m, 1H), 3.68-3.52 (m, 2H), 3.43 (s, 3H), 3.28-3.04 (m, 2H), 2.60-2.53 (m, 1H), 2.25-2.12 (m, 1H), 2.04-1.68 (m, 3H), 1.60-1.29 (m, 3H), 1.05-0.74 (m, 7H), 0.59 (t, J=7.5 Hz, 3H).

### DGKi Compounds 33 and 34

### 1-Methyl-4-((2S,5R)-2-methyl-5-propyl-4-(1-(4-(trifluoromethyl)phenyl)ethyl)piperazin-1-yl)-2-oxo-1,2-dihydropyrido[3,2-d]pyrimidine-6-carbonitrile

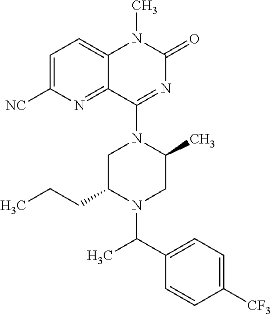 [SMILES: CCC[C@@H]1CN(c2nc(=O)n(C)c3ccc(C#N)nc23)[C@@H](C)CN1C(C)c1ccc(C(F)(F)F)cc1]

To a solution of 6-chloro-1-methyl-4-((2S,5R)-2-methyl-5-propyl-4-(1-(4-(trifluoromethyl)phenyl)ethyl)piperazin-1-yl)pyrido[3,2-d]pyrimidin-2(1H)-one (0.1 g, 0.19 mmol) in DMF (2 mL) were added zinc cyanide (0.046 g, 0.39 mmol), zinc (0.7 mg, 9.8 μmol) and triethylamine (0.1 mL, 0.59 mmol) followed by dichloro[9,9-dimethyl-4,5-bis(diphenylphosphino) xanthene]palladium(II) (0.015 g, 0.02 mmol) at room temperature under argon atmosphere. The reaction mixture was heated at 90 °C overnight. The reaction mixture was diluted with EtOAc (50 mL) and filtered through Celite® pad, washed with additional ethyl acetate (2×50 mL). The filtrate was washed with water (50 mL), brine, dried over Na₂SO₄ and concentrated under reduced pressure to yield the crude product, which was purified by preparative HPLC(HPLC method: Column: YMC EXRS (250×19 mm, 5 μm); mobile phase A: 10 mM ammonium acetate in water pH~4.5; mobile phase B: acetonitrile Flow: 20 mL/min) to yield Compound 33 and Compound 34.

COMPOUND 33: (13 mg, 14% yield). LCMS: m/z=499.3 [M+H]⁺; rt 2.376 min; (LCMS Method: Column: XBridge BEH XP C18 (50×2.1 mm, 2.5 μm); mobile phase A: 95% water: 5% acetonitrile; 10 mM NH₄OAc; mobile phase B: 5% water: 95% acetonitrile; 10 mM NH₄OAC; Flow: 1.1 mL/min; Temp: 50 °C). ¹H NMR (400 MHz, DMSO-d₆) δ (ppm)=8.22 (br d, J=8.8 Hz, 1H), 7.98 (d, J=8.8 Hz, 1H), 7.70-7.72 (m, 2H), 7.59-7.61 (m, 2H), 5.84-5.59 (m, 1H), 5.10-4.67 (m, 1H), 3.91-3.75 (m, 1H), 3.38-3.43 (m, 4H), 2.86-2.70 (m, 2H), 2.47-2.36 (m, 1H), 1.63-1.51 (m, 1H), 1.47-1.18 (m, 8H), 0.9-0.99 (m, 1H), 0.75-0.59 (m, 3H).

COMPOUND 34: (13 mg, 13% yield); LCMS: m/z=499.3 [M+H]⁺; rt 2.436 min; (LCMS Method: Column: XBridge BEH XP C18 (50×2.1 mm, 2.5 μm); mobile phase A: 95% water: 5% acetonitrile; 10 mM NH₄OAc; mobile phase B: 5% water: 95% acetonitrile; 10 mM NH₄OAC; Flow: 1.1 mL/min; Temp: 50 °C). ¹H NMR (400 MHz, DMSO-d₆) δ (ppm)=8.25 (br d, J=2.4 Hz, 1H), 8.06-7.92 (m, 1H), 7.77-7.65 (m, 2H), 7.65-7.54 (m, 2H), 6.09-5.44 (m, 1H), 5.04-4.68 (m, 1H), 3.81-3.59 (m, 2H), 3.44 (s, 3H), 3.28-3.13 (m, 1H), 2.52-2.61 (m, 1H), 2.24-2.05 (m, 1H), 1.72-1.48 (m, 2H), 1.47-1.15 (m, 8H), 0.98-0.75 (m, 3H).

### BIOLOGICAL ASSAYS

The pharmacological properties of the compounds described herein may be confirmed by a number of biological assays.

1. In vitro DGK Inhibition Assays

The DGKα and DGKζ reactions were performed using either extruded liposome (DGKα and DGKζ LIPGLO assays) or detergent/lipid micelle substrate (DGKα and DGKζ assays). The reactions were carried out in 50 mM MOPS pH 7.5, 100 mM NaCl, 10 mM MgCl₂, 1 μM CaCl₂, and 1 mM DTT (assay buffer). The reactions using a detergent/lipid micelle substrate also contained 50 mM octyl B-D-glucopyranoside. The lipid substrate concentrations were 11 mM PS and 1 mM DAG for the detergent/lipid micelle reactions. The lipid substrate concentrations were 2 mM PS, 0.25 mM DAG, and 2.75 mM PC for the extruded liposome reactions. The reactions were carried out in 150 μM ATP. The enzyme concentrations for the DGKα and DGKζ were 5 nM.

The compound inhibition studies were carried out as follows: 50 nL droplets of each test compound (top concentration 10 mM with 11 point, 3-fold dilution series for each compound) solubilized in DMSO were transferred to wells of a white 1536 well plate (Corning 3725). A 5 mL enzyme/substrate solution at 2× final reaction concentration was prepared by combining 2.5 mL 4× enzyme solution (20 nM DGKα or DGKζ (prepared as described below) in assay buffer) and 2.5 mL of either 4× liposome or 4× detergent/lipid micelle solution (compositions described below) and incubated at room temperature for 10 minutes. Next, 1 L 2× enzyme/substrate solution was added to wells containing the test compound and reactions were initiated with the addition of 1 μL 300 μM ATP. The reactions were allowed to proceed for 1 hr, after which 2 μL Glo Reagent (Promega V9101) was added and incubated for 40 minutes. Next, 4 μL Kinase Detection Reagent was added and incubated for 30 minutes. Luminescence was recorded using an EnVision microplate reader. The percent inhibition was calculated from the ATP conversion generated by no enzyme control reactions for 100% inhibition and vehicle-only reactions for 0% inhibition. The compounds were evaluated at 11 concentrations to determine IC₅₀.

### 4× Detergent/lipid Micelle Preparation

The detergent/lipid micelle was prepared by combining 15 g phosphatidylserine (Avanti 840035P) and 1 g diacylglycerol (8008110) and dissolving into 150 mL chloroform in a 2 L round bottom flask. Chloroform was removed under high vacuum by rotary evaporation. The resulting colorless, tacky oil was resuspended in 400 mL 50 mM MOPS pH 7.5, 100 mM NaCl, 20 mM NaF, 10 mM MgCl₂, 1 μM CaCl₂, 1 mM DTT, and 200 mM octyl glucoside by vigorous mixing. The lipid/detergent solution was split into 5 mL aliquots and stored at −80 °C.

### 4× Liposome Preparation

The lipid composition was 5 mol % DAG (Avanti 8008110), 40 mol % PS (Avanti 840035P), and 55 mol % PC (Avanti 850457) at a total lipid concentration of 15.2 mg/mL for the 4× liposome solution. The PC, DAG, and PS were dissolved in chloroform, combined, and dried in vacuo to a thin film. The lipids were hydrated to 20 mM in 50 mM MOPS pH 7.5, 100 mM NaCl, 5 mM MgCl₂, and were freeze-thawed five times. The lipid suspension was extruded through a 100 nm polycarbonate filter eleven times. Dynamic light scattering was carried out to confirm liposome size (50-60 nm radius). The liposome preparation was stored at 4 °C for as long as four weeks.

### Baculovirus Expression of Human DGKα and DGKζ

Human DGK-alpha-TVMV-His-pFBgate and human DGK-zeta-transcript variant-2-TVMV-His-pFBgate baculovirus samples were generated using the Bac-to-Bac baculovirus expression system (Invitrogen) according to the manufacturer's protocol. The DNA used for expression of DGK-alpha and DGK-zeta have SEQ ID NOs: 251 and 252, respectively. Baculovirus amplification was achieved using infected Sf9 cells at 1:1500 virus/cell ratios, and grown for 65 hours at 27 °C post-transfection.

The expression scale up for each protein was carried out in the Cellbag 50L WAVE-Bioreactor System 20/50 from GE Healthcare Bioscience. 12 L of 2×10⁶ cells/mL Sf9 cells (Expression System, Davis, CA) grown in ESF921 insect medium (Expression System) were infected with virus stock at 1:200 virus/cell ratios, and grown for 66-68 hours at 27 °C post-infection. The infected cell culture was harvested by centrifugation at 2000 rpm for 20 min 4 °C in a SORVALL® RC12BP centrifuge. The cell pellets were stored at −70 °C until purification.

### Purification of Human DGK-alpha and DGK-zeta

Full length human DGKα and DGKζ, each expressed containing a TVMV-cleavable C-terminal Hexa-His tag sequence (SEQ ID NOs: 190 and 191, respectively) and produced as described above, were purified from Sf9 baculovirus-infected insect cell paste. The cells were lysed using nitrogen cavitation method with a nitrogen bomb (Parr Instruments), and the lysates were clarified by centrifugation. The clarified lysates were purified to ~90% homogeneity, using three successive column chromatography steps on an ÄKTA Purifier Plus system. The three steps column chromatography included nickel affinity resin capture (i.e. HisTrap FF crude, GE Healthcare), followed by size exclusion chromatography (i.e. HiLoad 26/600 Superdex 200 prep grade, GE Healthcare for DGK-alpha, and HiPrep 26/600 Sephacryl S 300_HR, GE Healthcare for DGK-zeta). The third step was ion exchange chromatography, and differed for the two isoforms. DGKα was polished using Q-Sepharose anion exchange chromatography (GE Healthcare). DGKζ was polished using SP Sepharose cation exchange chromatography (GE Healthcare). The proteins were delivered at concentrations of ≥2 mg/mL. The formulation buffers were identical for both proteins: 50 mM Hepes, pH 7.2, 500 mM NaCl, 10% v/v glycerol, 1 mM TCEP, and 0.5 mM EDTA.

### 2. Raji CD4 T Cell IL2 Assay

A 1536-well IL-2 assay was performed in 4 μL volume using pre-activated CD4 T cells and Raji cells. Prior to the assay, CD4 T cells were pre-activated by treatment with α-CD3, α-CD28 and PHA at 1.5 μg/mL, 1 μg/mL, and 10 μg/mL, respectively. Raji cells were treated with Staphylococcal enterotoxin B (SEB) at 10,000 ng/mL. Serially diluted compounds were first transferred to 1536-well assay plate (Corning, #3727), followed by addition of 2 μL of pre-activated CD4 T cells (final density at 6000 cells/well) and 2 μL of SEB-treated Raji cells (2000 cells/well). After 24 hours incubation at a 37 °C/5% CO₂ incubator, 4 μl of IL-2 detection reagents were added to the assay plate (Cisbio, #64IL2PEC). The assay plates were read on an Envision reader. To assess compound cytotoxicity, either Raji or CD4 T cells were incubated with the serially diluted compounds. After 24 hours incubation, 4 μL of Cell Titer Glo (Promega, #G7572) were added, and the plates were read on an Envision reader. The 50% effective concentration (IC₅₀) was calculated using the four-parameter logistic formula y=A+((B−A)/(1+((C/x)\hat{ }D))), where A and B denote minimal and maximal % activation or inhibition, respectively, C is the IC₅₀, D is hill slope and x represent compound concentration.

### 3. CellTiter-Glo CD8 T Cell Proliferation Assay

Frozen naïve human CD8 T cells were thawed in RPMI+10% FBS, incubated for 2 h in 37 °C, and counted. The 384-well tissue culture plate was coated overnight at 4 °C with 20 μl anti-human CD3 at 0.1 μg/mL in plain RPMI, which was removed off the plate before 20 k/40 μL CD8 T cells with 0.5 μg/ml soluble anti-human CD28 were added to each well. The compounds were echoed to the cell plate immediately after the cells were plated. After 72 h incubation at 37 °C incubator, 10 μL CellTiter-glo reagent (Promega catalog number G7570) was added to each well. The plate was vigorously shaken for 5 mins, incubated at room temperature for another 15 mins and read on Envision for CD8 T cell proliferation. In analysis, 0.1 μg/mL anti-CD3 and 0.5 g/mL anti-CD28 stimulated CD8 T cell signal was background. The reference compound, 8-(4-(bis(4-fluorophenyl)methyl)piperazin-1-yl)-5-methyl-7-nitro-6-oxo-5,6-dihydro-1,5-naphthyridine-2-carbonitrile, at 3 μM was used to set the 100% range and EC₅₀ was at absolute 50% to normalize the data.

### 4. DGK AP1-Reporter Assay

The Jurkat AP1-luciferase Reporter was generated using the Cignal Lenti AP1 Reporter (luc) Kit from SABiosciences (CLS-011L).

The compounds were transferred from an Echo LDV plate to individual wells of a 384-well plate (white, solid-bottom, opaque PE CulturPlate 6007768) using an Echo550 instrument. The sample size was 30 nL per well; and one destination plate per source plate. The cell suspensions were prepared by transferring 40 mL cells (2×20 mL) to clean 50 mL conical tubes. The cells were concentrated by centrifugation (1200 rpm; 5 mins; ambient temperature). The supernatant was removed and all cells were suspended in RPMI (Gibco 11875)+10% FBS to make a 1.35×10⁶ cells/ml concentration. The cells were added manually using a multi-channel pipette, 30 μL/well of cell suspension to a 384-well TC plate containing the compounds, 4.0×10⁴ cells per well. The cell plates were incubated for 20 minutes at 37 °C and 5% CO₂.

During the incubation, anti-CD3 antibody (αCD3) solutions were prepared by mixing 3 μL aCD3(1.3 mg/mL) with 10 mL medium [final conc=0.4 μg/mL]. Next, 1.5 μl aCD3(1.3 mg/mL) was mixed with 0.5 mL medium [final conc=4 μg/ml]. After 20 minutes, 10 μL medium was added to all wells in column 1, wells A to M, and 10 μL αCD3(4 ug/mL) per well was added in column 1, rows N to P for reference. Then using a multi-channel pipette, 10 μL αCD3(0.4 ug/mL) per well was added. The αCD3 stimulated+/−compound-treated cells were incubated at 37 °C, 5% CO₂ for 6 hours.

During this incubation period, Steady-Glo (Promega E2520) reagent was slowly thawed to ambient temperature. Next, 20 μL Steady-Glo reagent per well was added using a multi-drop Combi-dispenser. Bubbles were removed by centrifugation (2000 rpm, ambient temperature, 10 secs). The cells were incubated at room temperature for 5 minutes. Samples were characterized by measuring the Relative Light Units (RLU) with an using Envision Plate Reader Instrument on a luminescence protocol. The data was analyzed using the reference compound, 8-(4-(bis(4-fluorophenyl)methyl)piperazin-1-yl)-5-methyl-7-nitro-6-oxo-5,6-dihydro-1,5-naphthyridine-2-carbonitrile, to normalize 100% inhibition.

### 5. Murine Cytotoxic T Lymphocyte Assay

An antigen-specific cytolytic T-cell (CTL) assay was developed to evaluate functionally the ability of DGKα and DGKζ inhibitors to enhance effector T cell mediated tumor cell killing activity. CD8⁺ T-cells isolated from the OT-1 transgenic mouse recognize antigen presenting cells, MC38, that present the ovalbumin derived peptide SIINFEKL. Recognition of the cognate antigen initiates the cytolytic activity of the OT-1 antigen-specific CD8+ T cells.

Functional CTL cells were generated as follows: OT-1 splenocytes from 8-12 week old mice were isolated and expanded in the presence of the SIINFEKL peptide at 1 μg/mL and mIL2 at 10 U/mL. After three days, fresh media with mIL2 U/ml was added. On day 5 of the expansion, the CD8+ T cells were isolated and ready for use. Activated CTL cells may be stored frozen for 6 months. Separately, one million MC38 tumor cells were pulsed with 1 μg/mL of SIINFEKL-OVA peptide for 3 hours at 37 °C. The cells were washed (3×) with fresh media to remove excess peptide. Finally, CTL cells that were pretreated with DGK inhibitors for 1 hour in a 96-well U bottom plate were combined with the antigen loaded MC38 tumor cells at a 1:10 ratio. The cells were then spun at 700 rpm for 5 min and placed in an incubator overnight at 37 °C After 24 hours, the supernatant was collected for analysis of IFN-γ cytokine levels by AlphaLisa purchased from Perkin Elmer.

### 6. PHA Proliferation Assay

Phytohaemagglutinin (PHA)-stimulated blast cells from frozen stocks were incubated in RPMI medium (Gibco, ThermoFisher Scientific, Waltham, MA) supplemented with 10% fetal bovine serum (Sigma Aldrich, St. Louis, MO) for one hour prior to adding to individual wells of a 384-well plate (10,000 cells per well). The compounds were transferred to individual wells of a 384-well plate and the treated cells are maintained at 37 °C, 5% CO₂ for 72 h in culture medium containing human IL2(20 ng/mL) prior to measuring growth using MTS reagent [3-(4,5-dimethyl-2-yl)-5-(3-carboxymethoxyphenyl)-2-(4-sulfophenyl)-2H-tetrazolium] following manufacturer's instructions (Promega, Madison, WI). Percent inhibition was calculated comparing values between IL2 stimulated (0% inhibition) and unstimulated control (100% inhibition). Inhibition concentration (IC₅₀) determinations were calculated based on 50% inhibition on the fold-induction between IL2 stimulated and unstimulated treatments.

### 7. Human CD8 T Cells IFN-γ Assay

Frozen naïve human CD8 T cells were thawed in AIM-V media, incubated for 2 h in 37 °C, and counted. The 384-well tissue culture plate was coated overnight at 4 °C with 20 μL anti-human CD3 at 0.05 g/mL in PBS, which was removed off the plate before 40,000 cells per 40 microliters CD8 T cells with 0.1 μg/mL soluble anti-human CD28 were added to each well. The compounds were transferred using an Echo liquid handler to the cell plate immediately after the cells were plated. After 20 h incubation at 37 °C incubator, 3 microliters per well supernatants transferred into a new 384-well white assay plate for cytokine measurement.

Interferon-γ (IFN-γ) was quantitated using the AlphLISA kit (Cat #AL217) as described by the manufacturer manual (Perkin Elmer). The counts from each well were converted to IFN-γ concentration (pg/mL). The compound EC₅₀ values were determined by setting 0.05 μg/mL anti-CD3 plus 0.1 μg/mL anti-CD28 as the baseline, and co-stimulation of 3 μM of the reference compound, 8-(4-(bis(4-fluorophenyl)methyl)piperazin-1-yl)-5-methyl-7-nitro-6-oxo-5,6-dihydro-1,5-naphthyridine-2-carbonitrile, with anti-CD3 plus anti-CD28 as 100% activation.

8. Human CD8 T Cells pERK Assay

Frozen naïve human CD8 T cells were thawed in AIM-V media, incubated for 2 h in 37 °C, and counted. The CD8 positive T cells were added to 384-well tissue culture plate at 20,000 cells per well in AIM-V media. One compound was added to each well, then bead bound anti-human CD3 and anti-CD28 mAb were added at final concentration of 0.3 μg/mL. The cells were incubated at 37 °C for 10 minutes. The reaction was stopped by adding lysis buffer from the AlphaLISA Surefire kit. (Perkin Elmer, cat #ALSU—PERK-A). Lysate (5 μL per well) was transferred into a new 384-well white assay plate for pERK activation measurement.

Compound EC₅₀ was determined as setting anti-CD3 plus anti-CD28 as baseline, and co-stimulation of 3 μM 8-(4-(bis(4-fluorophenyl)methyl)piperazin-1-yl)-5-methyl-7-nitro-6-oxo-5,6-dihydro-1,5-naphthyridine-2-carbonitrile with anti-CD3 plus anti-CD28 as 100% activation.

### 9. Human Whole Blood IFN-γ Assay

Human venous whole blood (22.5 μL per well), obtained from healthy donors, was pre-treated with compounds for one hour at 37 °C in a humidified 95% air/5% CO₂ incubator. The blood was stimulated with 2.5 μL anti-human CD3 and anti-CD28 mAb at a final concentration of 1 μg/mL each for 24 hours at 37 °C. IFN-γ in the supernatants was measured using AlphLISA kit (Cat #AL217).

Compound EC₅₀ determined as setting anti-CD3 plus anti-CD28 as baseline, and co-stimulation of 3 μM of the reference compound, 8-(4-(bis(4-fluorophenyl)methyl)piperazin-1-yl)-5-methyl-7-nitro-6-oxo-5,6-dihydro-1,5-naphthyridine-2-carbonitrile, with anti-CD3 plus anti-CD28 as 100% activation.

TABLE A
In Vitro DGK Inhibition IC₅₀ Activity Values

| | | | | | INFg |
|---|---|---|---|---|---|
| | | | HuCD8 | | Whole |
| | | | INFG | | Blood |
| Ex. | DGKa | DGKz | Normal- | msCTL | Normal- |
| DGKi | LIPGLO | LIPGLO | ized | INFg | ized |
| Compound | IC₅₀ | IC₅₀ | EC₅₀ | IC₅₀ | Agonist |
| No. | (μM) | (μM) | (μM) | (μM) | EC₅₀ (μM) |
| 1 | 23 | 42 | — | — | — |
| 2 | 240 | 1.7 | 0.062 | 0.064 | 0.52 |
| 4 | 0.98 | 0.85 | 0.017 | 0.043 | 0.40 |
| 5 | 0.87 | 1.6 | 0.030 | — | 0.68 |
| 6 | 0.66 | 0.42 | 0.039 | 0.033 | 0.70 |
| 7 | 0.46 | 0.51 | 0.047 | 0.064 | 0.57 |
| 8 | 15 | 20 | 12 | — | — |
| 9 | 4.4 | 0.97 | 0.17 | — | 1.5 |
| 10 | 0.67 | 0.32 | 0.023 | 0.032 | 0.44 |
| 11 | 0.14 | 0.57 | 0.022 | — | 0.65 |
| 12 | 0.57 | 0.46 | 0.048 | — | 0.44 |
| 13 | 0.15 | 0.33 | 0.020 | — | 0.13 |
| 14 | 1.7 | 3.7 | — | — | — |
| 15 | 3.4 | 0.21 | — | — | — |
| 16 | 1.5 | 0.87 | 0.0095 | 0.072 | 0.52 |
| 17 | 0.16 | 0.13 | — | — | 0.02 |
| 18 | 4.66 | 2.44 | — | — | 3.75 |
| 19 | 0.39 | 0.81 | — | — | 0.52 |
| 20 | 0.12 | 0.044 | — | — | 0.032 |
| 21 | 0.094 | 0.060 | — | — | 0.043 |
| 22 | 0.099 | 0.054 | — | — | 0.020 |
| 23 | 0.15 | 0.29 | — | — | 10.48 |
| 24 | 0.32 | 0.13 | — | — | 0.20 |
| 25 | 0.067 | 0.029 | — | — | 0.015 |
| 26 | 0.014 | 0.033 | — | — | 0.090 |
| 27 | 0.19 | 0.44 | — | — | — |
| 28 | 0.18 | 0.56 | — | — | 0.24 |
| 29 | — | 0.038 | — | — | — |
| 30 | — | 0.92 | — | — | 11.3 |
| 31 | — | 0.039 | — | — | — |
| 33 | — | 0.064 | — | — | 20 |
| 34 | — | 1.30 | — | — | — |

Table A lists in vitro DGK inhibition IC₅₀ activity values measured in the DGKα and DGKζ liposome (LIPGLO) assays.

The compounds described herein possess activity as an inhibitor(s) of one or both of the DGKα and DGKζ enzymes, and therefore, may be used in the treatment of diseases associated with the inhibition of DGKα and DGKζ activity.

### Example 2: Effects of Compound 17 on CAR-T Cell Function

T cell function of T cells engineered with a chimeric antigen receptor (CAR-T cells) treated with an exemplary DGKi (Compound 17) was assessed.

T cell compositions containing T cells expressing an exemplary anti-CD19 CAR were generated from leukapheresis samples from two healthy human adult donors by a process that included immunoaffinity-based selection of T cells by separately selecting CD4+ and CD8+ cells from the samples, resulting in two compositions enriched for CD4+ and CD8+ cells, respectively, per donor. Cells of the enriched CD4+ and CD8+ compositions were separately activated with anti-CD3/anti-CD28 beads and subjected to lentiviral transduction with a vector encoding an anti-CD19 CAR. The anti-CD19 CAR contained an anti-CD19 single-chain variable fragment (scFv) derived from a murine antibody (variable region derived from FMC63), an immunoglobulin-derived spacer, a transmembrane domain derived from CD28, a costimulatory region derived from 4-1BB, and a CD3-zeta intracellular signaling domain. The expression construct in the viral vector further contained sequences encoding a surrogate surface marker for CAR expression, which was separated from the CAR-encoding sequence by a T2A ribosome skip sequence. Transduced CD4+ or CD8+ T cell populations then were separately incubated in the presence of stimulating reagents for cell expansion. Expanded CD4+ and CD8+ cells were formulated and cryopreserved separately and stored.

Prior to use, the cryopreserved CD4+ and CD8+ CAR-engineered cell compositions were thawed and combined at approximately a 1:1 CD4:CD8 ratio for each donor to prepare a combined CD4+ CAR+ and CD8+ CAR+ T cell composition. The effects of treatment with Compound 17 at various concentrations (0.1 nM, 1 nM, 10 nM, 100 nM, or 1000 nM) added at day 0 during stimulation of the combined CAR-T cell composition with a plate-bound CAR-specific anti-idiotypic antibody (see, e.g., WO2018/023100) or during co-culture with CD19-expressing cells was then assessed.

### A. T Cell Phenotype

Approximately 20,000 cells of the combined CAR-T cell composition were stimulated with 3 μg/ml or 30 μg/ml plate-bound CAR-specific anti-idiotypic antibody in the presence of Compound 17. After a 48-hour stimulation, T cells were surface stained with antibodies for various activation and differentiation markers and analyzed by flow cytometry for the percent of cells positive for the assessed markers (% positive) and for mean fluorescence intensity (MFI) of expression for the assessed markers. The mean log 2 fold change value for % positive cells or MFI from the two healthy donors for expression of the surface marker compared to expression in the absence of treatment with Compound 17 was determined.

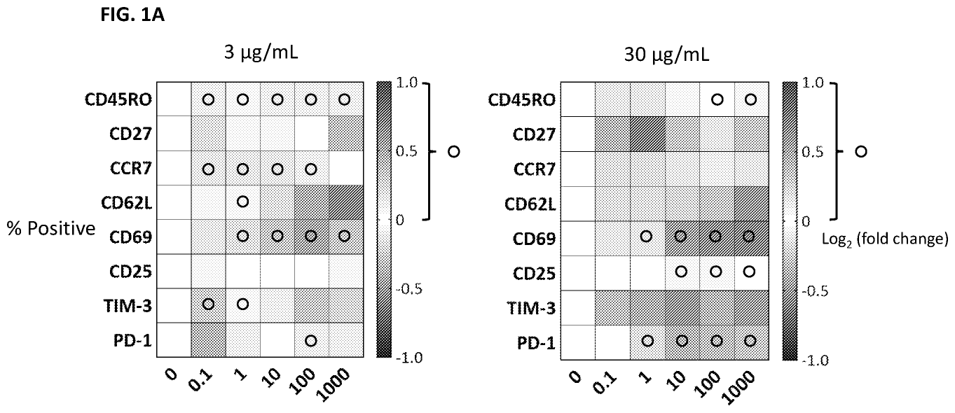

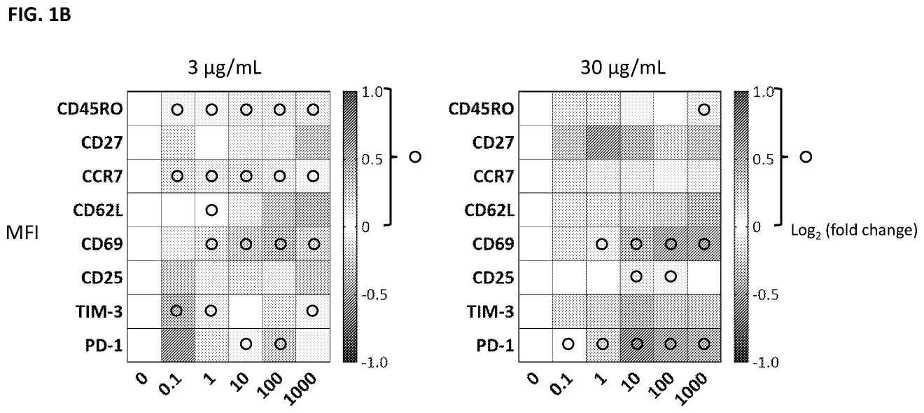

FIG. 1A shows the mean across donors of the log 2 fold change (presence vs. absence of Compound 17) in the % positive cells for the various activation and differentiation markers following stimulation with 3 μg/mL (left panel) or 30 μg/mL (right panel) of the CAR-specific anti-idiotypic antibody. FIG. 1B shows the mean across donors of the log 2 fold change (presence vs. absence of Compound 17) in MFI of cells following stimulation with 3 μg/mL (left panel) or 30 μg/mL (right panel) of the CAR-specific anti-idiotypic antibody. In FIGS. 1A and 1B, positive values are indicated with circles. As shown in both FIG. 1A and FIG. 1B, CAR-expressing cells stimulated in the presence of Compound 17 had increased expression of CD69 and PD-1 and decreased expression of CD27 and CD62L, consistent with more cells having an activated effector phenotype following stimulation in the presence of Compound 17. This effect on cell phenotype was dose-dependent, in particular for cells stimulated with a higher concentration of the CAR-specific anti-idiotypic antibody (30 μg/mL).

### B. Proliferation

T cell proliferation during stimulation with 30 μg/mL of plate-bound anti-CAR anti-idiotypic antibody in the presence of Compound 17 was monitored for seven days using an Incucyte imaging system. Cell proliferation was monitored by analyzing the occupied area (% confluence) of cell images over time; as cells proliferate, the confluence increases.

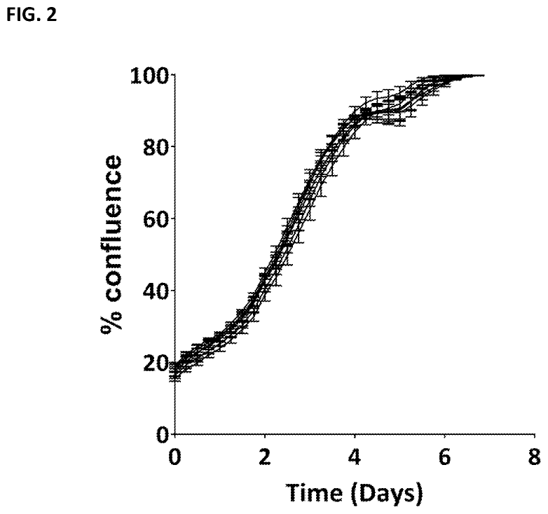

FIG. 2 shows growth curves (percent cellular confluence in well) of cells stimulated in the presence of Compound 17 or a control. Triplicate values for cell compositions manufactured from the two different healthy donors are shown. As shown, the presence of Compound 17 during stimulation did not affect T cell proliferation.

### C. Cytolytic Function

The cytolytic function of T cells treated with Compound 17 was assessed following co-culture of the combined CAR-T cell composition with various CD19-expressing lymphoma cells at a 2.5:1 effector:target ratio in the presence of Compound 17. The target lymphoma cells were labeled with NucLight Red (NLR) to permit their tracking by microscopy. Cytolytic activity was assessed by measuring the loss of viable target cells over a period of four to seven days, as determined by red fluorescent signal (using the IncuCyte® Live Cell Analysis System, Essen Bioscience).

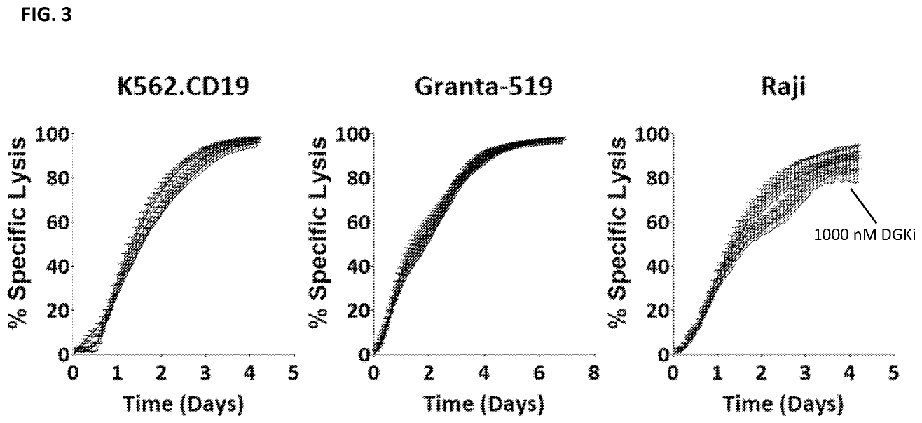

FIG. 3 shows the percent of specific lysis effected by CAR-expressing cells in the presence of Compound 17 (or a control) when co-cultured with CD19-antigen expressing target cells, K562.CD19 cells (high CD19 expression; left panel), Granta-519 cells (low CD19 expression; middle panel), and Raji cells (medium/high CD19 expression; right panel). Triplicate values for cell compositions manufactured from the two different healthy donors are shown. As shown, the presence of Compound 17 during co-culture did not affect cytolytic function.

### D. Cytokine Production

Supernatant was collected after 48 hours from the above co-cultures of CAR-expressing T cells and CD19-expressing tumor cells in the presence of Compound 17. Cytokine production was measured from the supernatant by meso scale discovery (MSD) analysis of the collected cell supernatants.

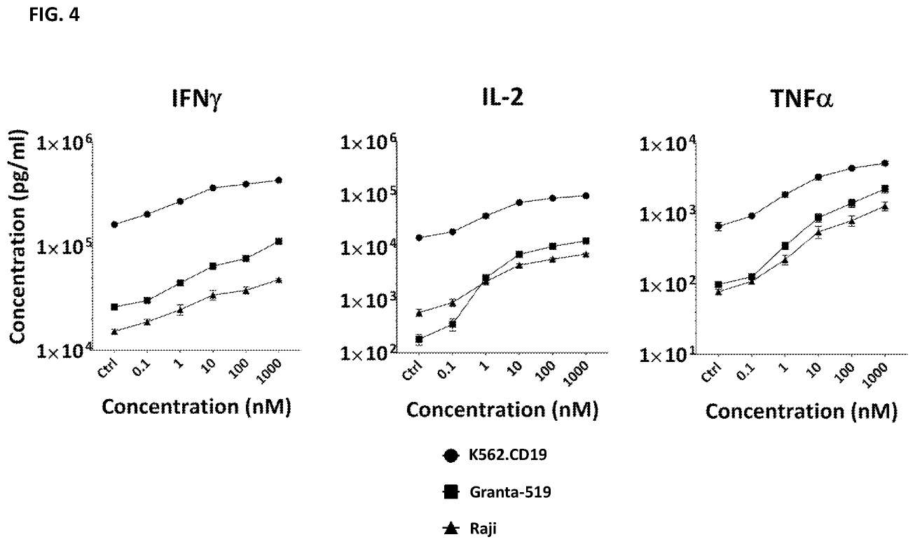

FIG. 4 shows the supernatant concentrations in pg/mL of IFNγ (left panel), IL-2 (middle panel), and TNFα (right panel) following co-culture with K562.CD19 cells (circles), Granta-519 cells (squares), and Raji cells (triangles) in the presence of Compound 17. As shown, the presence of Compound 17 during co-culture led to a dose-dependent increase in production of all three cytokines, with higher concentrations of Compound 17 leading to the greatest effect during co-culture with Granta-519 cells (low CD19 expression), particularly for IL-2 production.

### E. Summary

Together, these results show that without affecting CAR T cell proliferation or cytolytic function, treatment with Compound 17 increased cytokine production of CAR T cells as well as the number of CD4+ and CD8+ CAR-expressing cells having an activated effector phenotype. Further, no impact of Compound 17 on T cell viability was observed in these assays. These results are consistent with an observation that under conditions of acute stimulation, the presence of Compound 17 potentiates CAR-T cell activation.

### Example 3: Assessment of Chronically Stimulated CAR-T Cells Concurrently Treated with Compound 17

The ability of Compound 17 to block or reduce exhaustion in chronically stimulated CAR T cells was assessed.

To simulate conditions of chronic stimulation, approximately 20,000 anti-CD19 CAR-expressing T cells of a combined CAR-T cell composition produced as described in Example 2 from two healthy adult donors were incubated with 30 g/mL of plate-bound CAR-specific anti-idiotypic antibody (see, e.g., WO2018/023100) at 37 °C for a period of seven days. During this seven-day period, engineered CAR T cells were concurrently treated with Compound 17(0.1-1000 nM) or a vehicle control added at day 0. Similar to results observed in Example 2 following acute stimulation, concurrent incubation of T cells with Compound 17 during the chronic stimulation increased populations of highly activated CAR-T cells among the stimulated cells, particularly cell populations high in expression of CD69 and PD-1.

Stimulated CAR T cells were then re-challenged with CD19-expressing tumor cells or tumor spheroids in the absence of Compound 17, and cytolytic activity and cytokine production were assessed.

### A. 2-Dimensional CD19+ Tumors

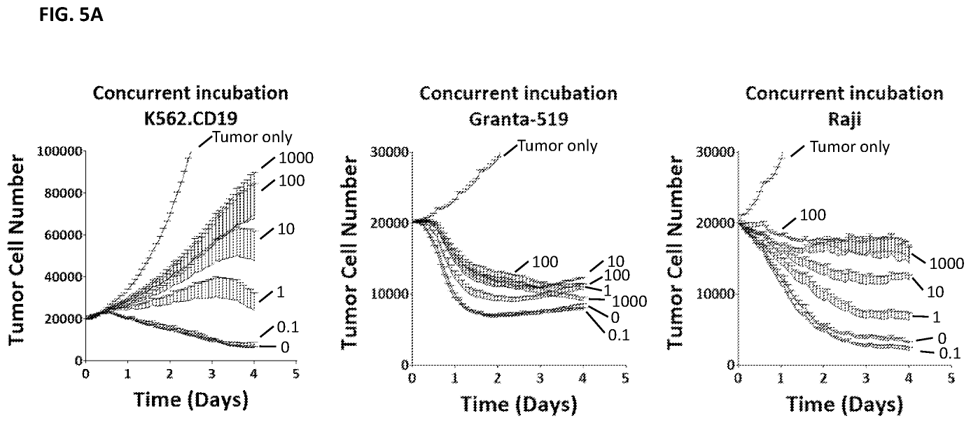

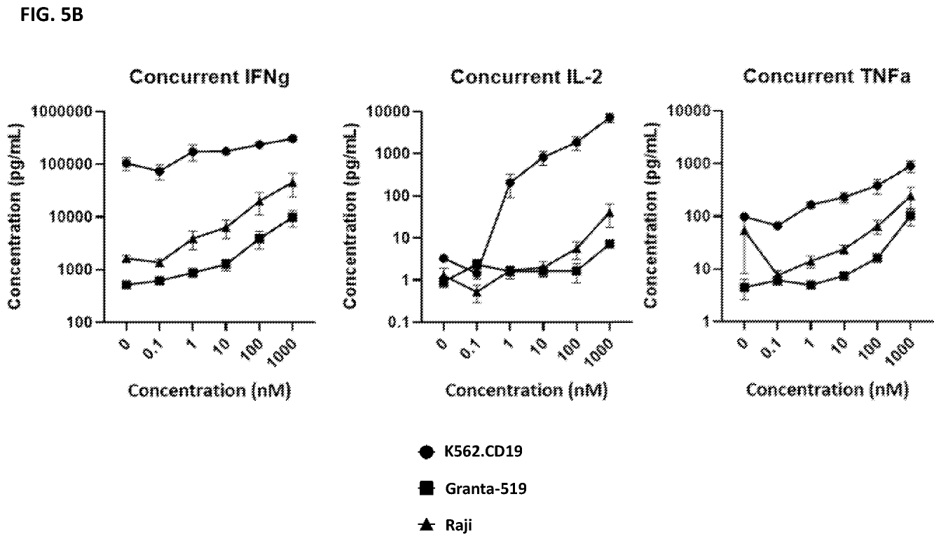

To assess cytolytic function, the stimulated CAR-T cell composition was co-cultured with various CD19-expressing lymphoma cells at a 2.5:1 effector:target ratio. The tumor cells were engineered to express NucLight Red (NLR) and tumor cell number was monitored by Incucyte®. FIG. 5A shows tumor cell numbers of K562.CD19 (left panel), Granta-519 (middle panel), and Raji cells (right panel) during 2D co-culture with CAR T cells that had been chronically stimulated in the presence of concurrent treatment with Compound 17. Duplicate values for cell compositions manufactured from the two different healthy donors are shown. As shown, incubation with the DGKi during chronic stimulation of the T cells decreased CAR-T cell cytolytic activity against the CD19-expressing lymphoma cells in a dose-dependent manner. In particular, treatment with higher concentrations of Compound 17 during chronic stimulation decreased the cytolytic activity of the CAR T cells.

Supernatant was collected after 48 hours from the above co-cultures of chronically stimulated CAR-expressing T cells and CD19-expressing tumor cells. Cytokine production was measured from the supernatant by meso scale discovery (MSD) analysis of the collected cell supernatants. FIG. 5B shows the supernatant concentrations in pg/mL of IFNγ (left panel), IL-2 (middle panel), and TNFα (right panel) following 48 hours of 2D co-culture with K562.CD19 cells (circles), Granta-519 cells (squares), and Raji cells (triangles). Duplicate values for cell compositions manufactured from the two different healthy donors are shown. The results show that treatment with Compound 17 during chronic stimulation led to a dose-dependent increase in production of all three cytokines.

### B. 3-Dimensional Spheroid CD19+ Tumors

To further assess activity of chronically stimulated cell, the chronically stimulated CAR-T cells that had been concurrently treated with DGKi were incubated with A549 CD19+ tumor spheroids or Granta-519 tumor spheroids to rechallenge the CAR-T cells at an effector to target ratio of 1:2 for up to 14 days. The tumor spheroids were engineered to express a red fluorescent dye to permit monitoring of tumor cell lysis by microscopy during the assay.

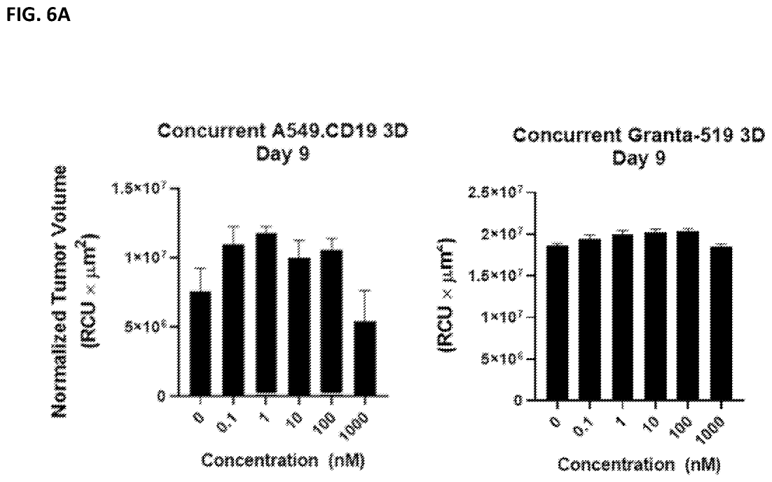

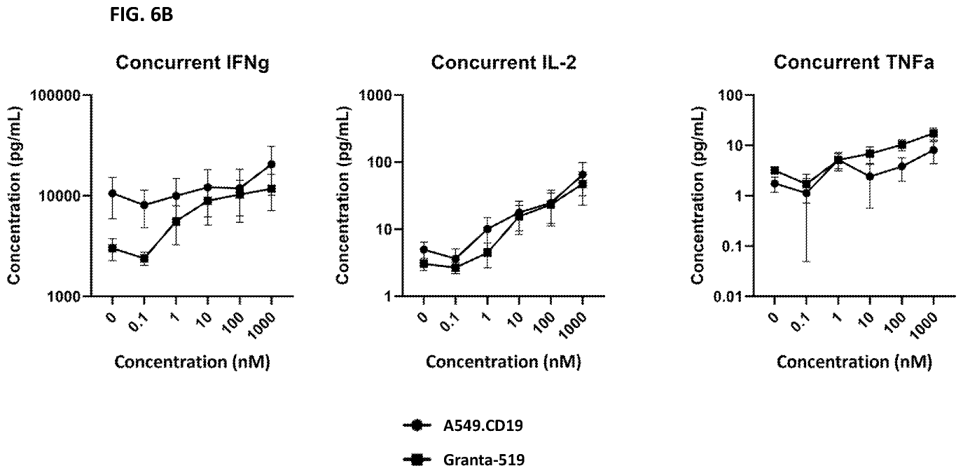

The antigen-specific cytolytic activity of the chronically stimulated CAR-expressing T cells was assessed by monitoring fluorescence (indicative of tumor cell lysis) over time. The tumor volume (normalized to time zero) was measured as the number of red calibrated units (RCU)×μm² area. FIG. 6A shows normalized tumor volume of A549.CD19 (left panel) and Granta-519 tumor spheroids (right panel) at day 9 of co-culture with CAR T cells that had been concurrently treated with Compound 17 during chronic stimulation. Duplicate values for cell compositions manufactured from the two different healthy donors are shown. In 3D cultures (FIG. 6A), treatment with Compound 17 during chronic stimulation modestly reduced the cytolytic activity of the CAR T cells.

Supernatant was collected at day 5 from the above tumor spheroid cultures with chronically stimulated CAR-expressing T cells. Cytokine production was measured from the supernatant by meso scale discovery (MSD) analysis of the collected cell supernatants. FIG. 6B shows the supernatant concentrations in pg/mL of IFNγ (left panel), IL-2 (middle panel), and TNFα (right panel) following five days of 3D co-culture with A549.CD19 (circles) and Granta-519 tumor spheroids (squares). Duplicate values for cell compositions manufactured from the two different healthy donors are shown. As shown, treatment with Compound 17 during chronic stimulation led to a dose-dependent increase in production of all three cytokines, similar to the results in the 2-dimensional tumor cell cultures.

### Example 4: Assessment of Chronically Stimulated CAR-T Cells Following Rescue Treatment with Compound 17

The ability of Compound 17 to rescue chronically stimulated CAR T cells from exhaustion was assessed.

Anti-CD19 CAR-expressing T cells, produced as described in Example 2, were chronically stimulated with plate-bound CAR-specific anti-idiotypic antibody for seven days as described in Example 3, but in the absence of Compound 17 during chronic stimulation. Instead, chronically stimulated CAR T cells were treated with Compound 17 during re-challenge with CD19-expressing tumor cells, after which cytolytic activity and cytokine production were assessed.

### A. 2-Dimensional CD19+ Tumors

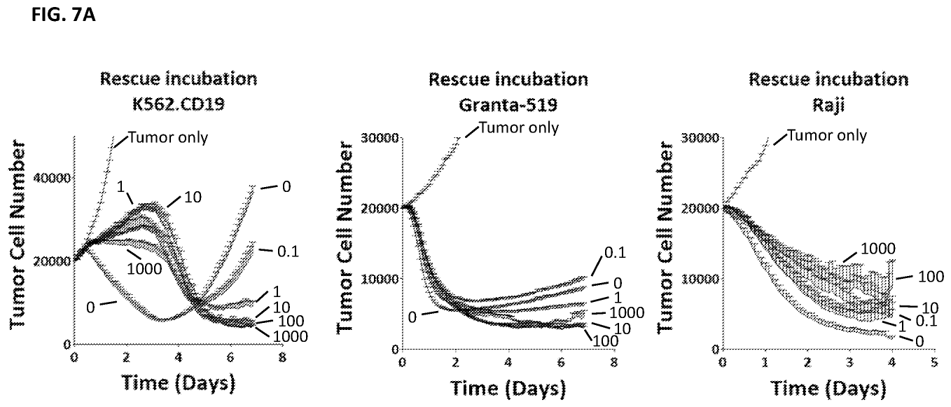

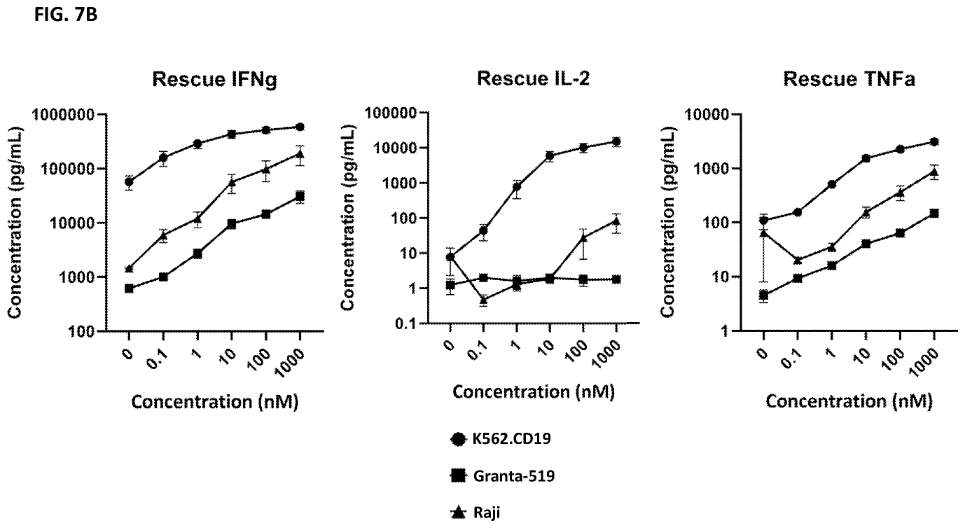

The chronically stimulated CAR-T cells were incubated with various CD19-expressing lymphoma cells at a 2.5:1 effector:target ratio in the presence of DGKi (0-1000 nM). The tumor cells were labeled with NucLight Red (NLR) and tumor cell number was monitored by Incucyte®. FIG. 7A shows tumor cell numbers of K562.CD19 (left panel), Granta-519 (middle panel), and Raji cells (right panel) during 2D co-culture with CAR T cells in the presence of Compound 17. As shown in FIG. 7A, rescue treatment with higher concentrations of Compound 17 during co-culture improved the cytolytic activity of CAR T cells against K562.CD19 and Granta-519 cells, but not Raji cells.

Supernatant was collected after 48 hours from the above co-cultures of chronically stimulated CAR-expressing T cells and CD19-expressing tumor cells. Cytokine production was measured from the supernatant by meso scale discovery (MSD) analysis of the collected cell supernatants. FIG. 7B shows the supernatant concentrations in pg/mL of IFNγ (left panel), IL-2 (middle panel), and TNFα (right panel) following 48 hours of 2D co-culture with K562.CD19 cells (circles), Granta-519 cells (squares), and Raji cells (triangles) in the presence of Compound 17. Duplicate values for cell compositions manufactured from the two different healthy donors are shown. As shown, rescue treatment with Compound 17 during co-culture led to a dose-dependent increase in production of all three cytokines, with the exception of IL-2 production during 2D culture with Granta-519 cells.

### B. 3-Dimensional Spheroid CD19+ Tumors

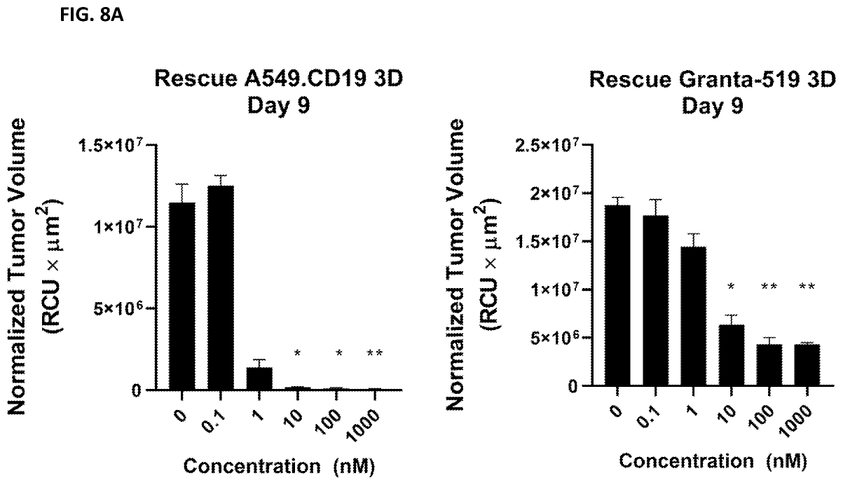

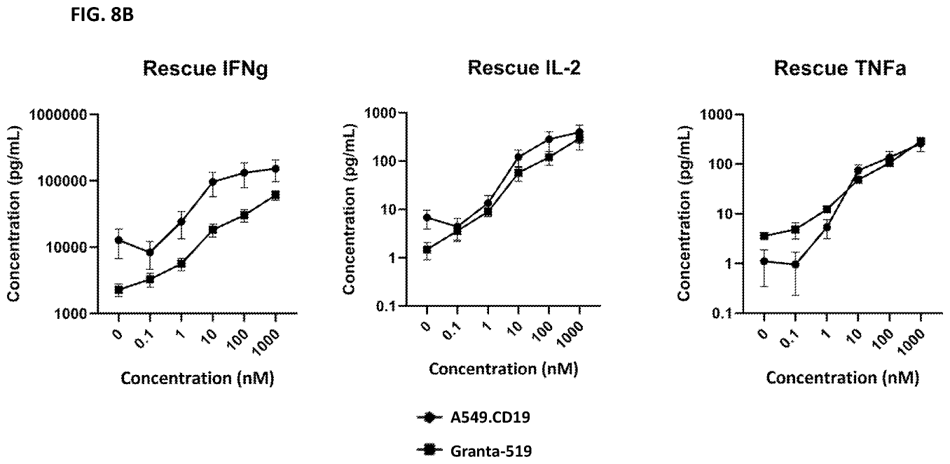

Cytolytic activity also was assessed in CD19-expressing tumor spheroid cultures incubated with chronically stimulated CAR-T cells in the presence of DGKi. A549 CD19+ tumor spheroids or Granta-519 tumor spheroids were incubated with the anti-CD19 CAR-expressing T cells at an effector to target ratio of 1:2 for up to 14 days in the presence of DGKi (0-1000 nM). The tumor spheroids were engineered to express a red fluorescent dye to permit monitoring of tumor cell lysis by microscopy during the assay. Cytolytic activity was assessed by monitoring fluorescence over time, and the tumor volume (normalized to time zero) was measured as the number of red calibrated units (RCU)×μm² area. FIG. 8A shows normalized tumor volume of A549.CD19 (left panel) and Granta-519 tumor spheroids (right panel) at day 9 of co-culture with CAR T cells in the presence of Compound 17. Duplicate values for cell compositions manufactured from the two different healthy donors are shown. As shown, rescue treatment with Compound 17 during co-culture improved the cytolytic activity of CAR T cells against both A549.CD19 and Granta-519 tumor spheroids.

Supernatant was collected at day 5 from the above tumor spheroid cultures with chronically stimulated CAR-expressing T cells. Cytokine production was measured from the supernatant by meso scale discovery (MSD) analysis of the collected cell supernatants. FIG. 8B shows the supernatant concentrations in pg/mL of IFNγ (left panel), IL-2 (middle panel), and TNFα (right panel) following five days of 3D co-culture with A549.CD19 (circles) and Granta-519 tumor spheroids (squares) in the presence of Compound 17. Duplicate values for cell compositions manufactured from the two different healthy donors are shown. Rescue treatment with Compound 17 during spheroid co-culture led to a dose-dependent increase in production of all three cytokines.

### C. Summary

Together, these results show that rescue treatment with Compound 17 during re-challenge of CAR T cells can improve cytolytic function and cytokine production, particularly in tumor spheroid cultures. Together with the results in the above examples, these results are consistent with a conclusion that delayed treatment with Compound 17 can enhance or increase T cell performance in response to CAR antigen, and can reduce or reverse an exhausted or chronically stimulated state in CAR T cells.

### Example 5: Assessment of Treatment with Compound 17 on Chronically Stimulated eTCR Cells

The ability of Compound 17 to rescue chronically stimulated T cells engineered with a T cell receptor (eTCR) from exhaustion was assessed.

Primary human CD4+ and CD8+ T cells were isolated from healthy donors by immunoaffinity-based methods. The isolated CD4+ and CD8+ T cells were combined and stimulated with an anti-CD3/anti-CD28 reagent, and then were transduced with a lentiviral vector encoding an anti-human papillomavirus 16 (anti-HPV16) eTCR. The nucleotide sequences encoding the beta chain and the alpha chain of the eTCR were separated by a sequence encoding a 2A ribosome skip element. Following transduction, the cells were cultured in media containing human serum and cytokines for 9-10 days prior to further assessment.

To assess the effects of rescue treatment with Compound 17 on cytolytic function, eTCR cells were stimulated for six days using 10 μg/mL or 20 μg/mL plate-bound antibody against the TCR Vbeta chain in the absence of Compound 17.

Following chronic stimulation, the eTCR-T cells were co-cultured with HPV-expressing CaSki cells at a 2.5:1 effector:target ratio in the presence of Compound 17 at 100 nM. As a control, chronically stimulated eTCR cells were co-cultured with CaSki cells in the absence of Compound 17. The CaSki cells were transduced with NucLight Red (NLR) and tumor cell number was monitored by Incucyte®.

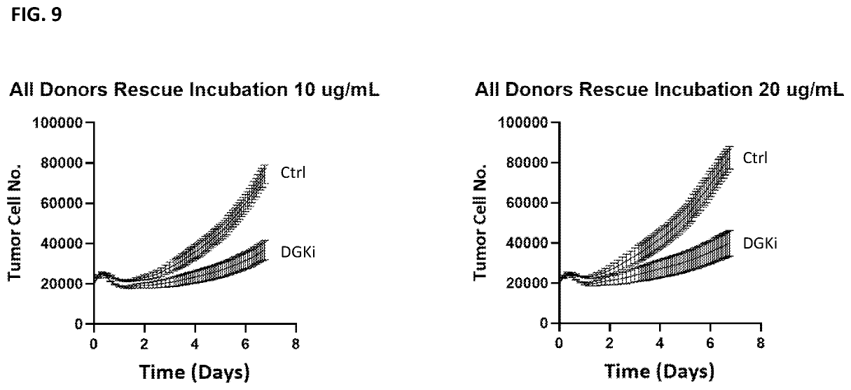

FIG. 9 shows tumor cell numbers of CaSki cells monitored at various times during co-culture with chronically stimulated eTCR T cells in the presence of Compound 17 (left panel, eTCR T cells chronically stimulated with 10 μg/mL anti-Vbeta; right panel eTCR T cells chronically stimulated with 20 μg/mL anti-Vbeta). As shown, rescue treatment with Compound 17 present during the culture with tumor cells was able to improve cytolytic function of chronically stimulated eTCR cells. This result is consistent with observations shown in Example 4 for CAR-expressing T cells, indicating that DGK inhibition is able to reverse or rescue an exhausted or chronically stimulated state of T cells engineered with different antigen receptors.

In another experiment to assess activity of the DGKi to rescue or reverse exhaustion, eTCR T cells were chronically stimulated for 6 days as described above in the absence of Compound 17. Then, chronically stimulated eTCR T cells or freshly thawed eTCR T cells that had not been chronically stimulated were re-stimulated with 20 μg/mL plate-bound antibody against the Vbeta chain of the eTCR in the presence of 100 nM Compound 17 for 7 days. As a control, chronically stimulated eTCR T cells were re-stimulated in the absence of Compound 17 for 7 days. At the end of the 7 day re-stimulation, cell counts were determined.

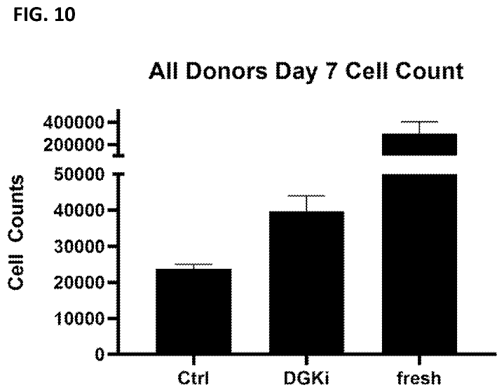

FIG. 10 shows the cell counts for eTCR T cells for two donors, as shown from left to right: (1) chronically stimulated eTCR T cells with no compound treatment (control); (2) chronically stimulated eTCR T cells treated with Compound 17; or (3) fresh eTCR T cells that were not chronically stimulated. As expected, in the absence of treatment with Compound 17, there was an overall reduction in the ability of chronically stimulated T cells to proliferate compared to fresh eTCR T cells (compare conditions (1) and (3)). However, treatment of chronically stimulated T cells with Compound 17 led to increased proliferation of the eTCR T cells. These results further confirm the ability of the DGKi to reverse or rescue T cells from exhaustion during subsequent antigen receptor-dependent re-stimulation.

### Example 6: Assessment of Dosing Regimens for Compound 17 in Combination with CAR-T Cells

The in vivo anti-tumor effects of different dosing regimens for Compound 17 in combination with anti-CD19 CAR T cells were assessed. To produce the anti-CD19 CAR T cells, T cells from a healthy human donor were engineered with a CD19-directed CAR.

For assessing in vivo anti-tumor effects, NOD.Cg.Prkdc^{scid}IL2rg^{tm/Wjl}/SzJ (NSG) mice were injected intravenously with 0.5×10⁶ Raji lymphoma tumor cells (an immortalized human B lymphocyte tumor cell line that expresses CD19) that were transduced with red-shifted firefly luciferase (Raji-rFluc cells). Tumor engraftment was allowed to occur for 11 days, after which on Day 1 of the combination therapy, mice received a single intravenous injection of 1×10⁶ anti-CD19 CAR-expressing T cells (81.8% of total administered T cells) or were left untreated. This dose of CAR-expressing T cells was not expected to achieve complete tumor rejection.

Following CAR-T cell administration, mice received one of three different Compound 17 dosing regimens or a vehicle control (n=8 per group). Specifically, mice received Compound 17 or vehicle control once daily (i) on Days 1-14 (“Early”), (ii) on Days 14-42 (“Delayed”), or (iii) on Days 1-28 (“Continuous”) of the combination therapy. All doses of Compound 17 were 0.3 mg/kg and were administered orally. For assessing tumor burden by bioluminescence imaging, mice received intraperitoneal injections of luciferin substrate resuspended in phosphate-buffered saline. Tumor burden and survival of treated mice were assessed until Day 50. In addition, flow cytometry for assessing circulating CAR T cell number was performed at Day 15 and Day 29. Flow cytometry for assessing CD4:CD8 ratio was performed at Day 15. CAR expression was assessed using an anti-idiotypic (anti-ID) antibody specific for the CAR.

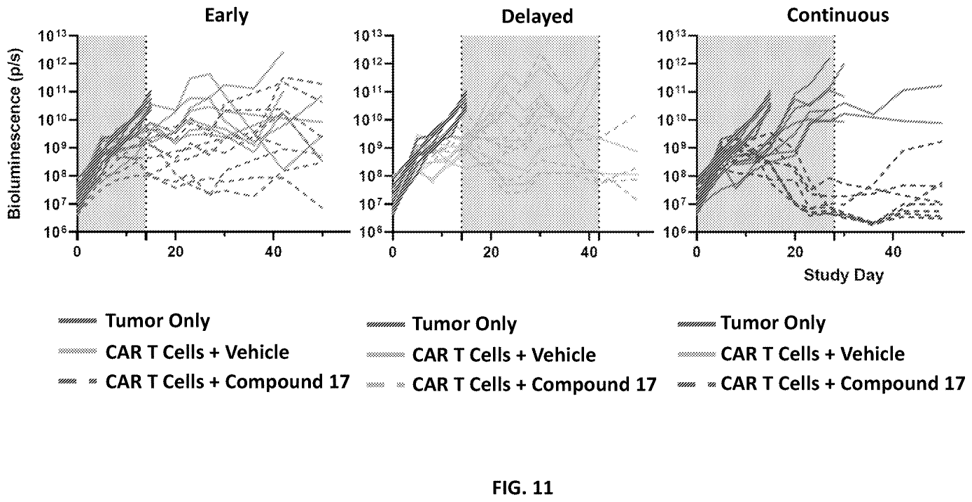

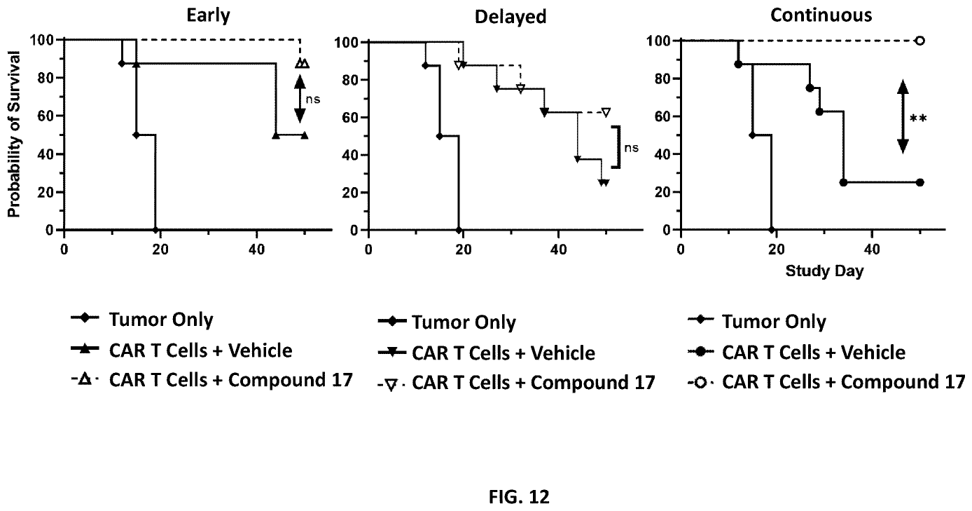

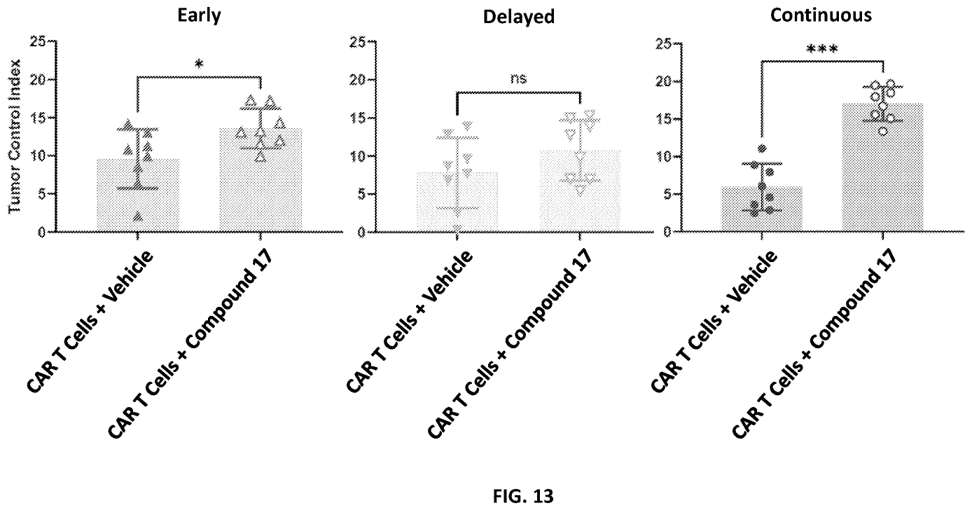

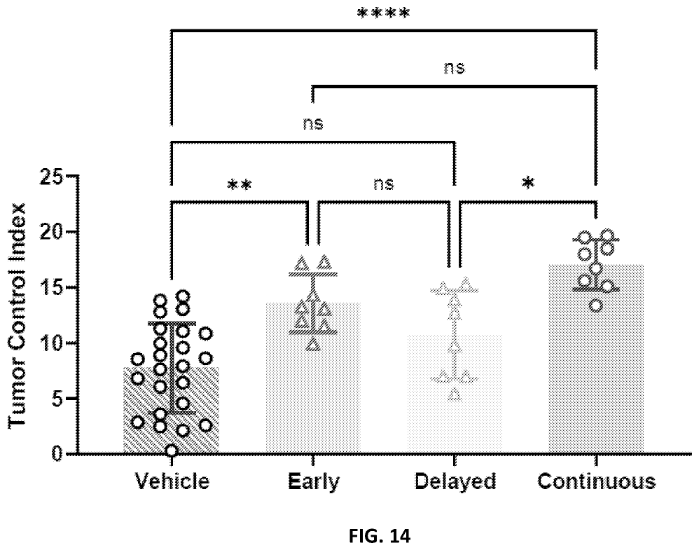

FIG. 11 and FIG. 12 show tumor burden and survival, respectively, in mice receiving the three different Compound 17 dosing regimens in combination with the anti-CD19 CAR T cells. As shown in FIG. 12, administration of the Continuous regimen improved survival, whereas administration of the Early and Delayed regimens did not. As indicated by the tumor control index values shown in FIG. 13 and FIG. 14, co-administration of Compound 17 during Days 1-14 (e.g., during periods of time in which CAR T cells can expand in vivo following administration), as in the Early and Continuous regimens, resulted in enhanced tumor suppression relative to vehicle-treated mice, whereas administration of the Delayed regimen did not. Tumor control index scores represent a composite parameter derived from tumor growth data.

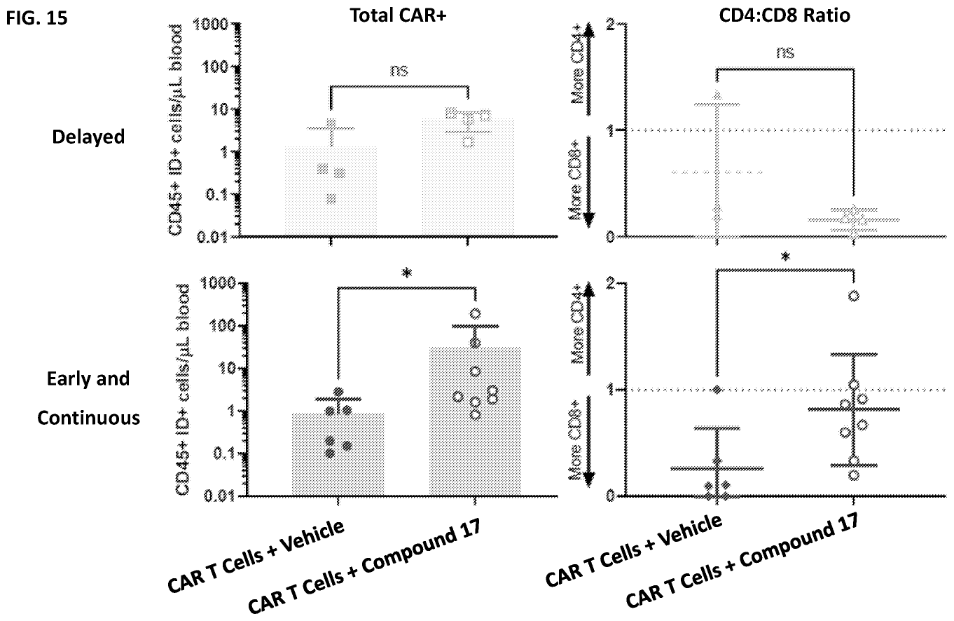

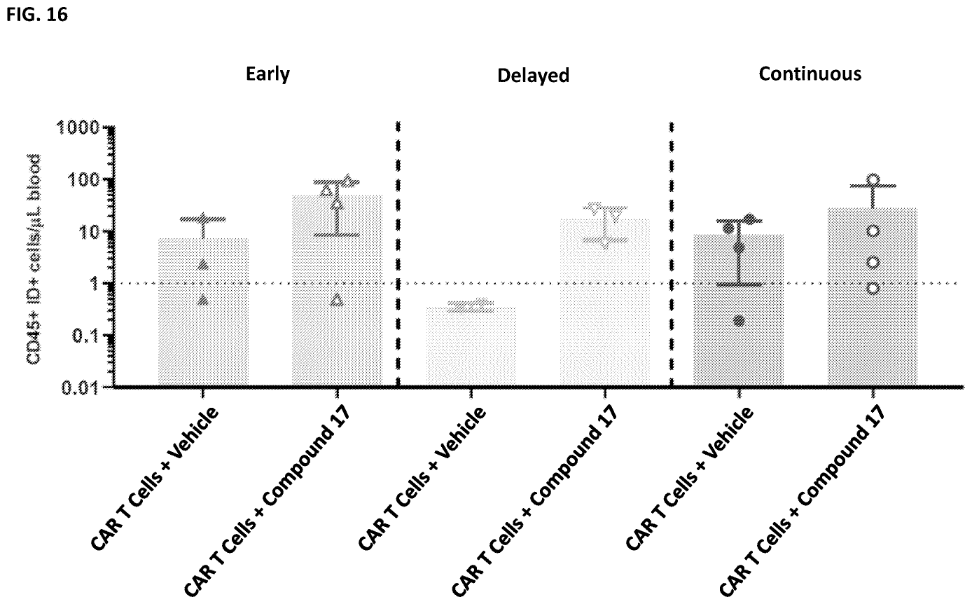

FIG. 15 shows the number of circulating CAR-expressing cells and the CD4:CD8 ratio of circulating T cells on Day 15 in mice receiving the Delayed regimen or either the Early or Continuous regimens. At this timepoint, mice receiving the Delayed regimen had received one prior dose of Compound 17. As shown in FIG. 15, administration of the Early or Continuous regimen resulted in increased CAR T cell expansion and an increase in CD4:CD8 ratio relative to vehicle-treated mice, whereas administration of the single dose of the Delayed regimen did not. The number of circulating CAR-expressing cells on Day 29 is shown in FIG. 16.

Together, these results indicate that administration of a DGKi beginning on the same day as administration of engineered cells for a T cell therapy and continuing once daily on Days 1-28 following T cell therapy administration reduced tumor burden and improved survival. As demonstrated herein, the presence of the DGKi following T cell therapy administration (e.g., beginning on Day 1) can improve CAR T cell expansion and/or persistence, in particular that of CD4+ CAR T cells, which may contribute to the demonstrated anti-tumor effects. The continued presence of the DGKi after Day 14 may also contribute to the demonstrated anti-tumor effects. Without wishing to be limited by a specific mechanism of action, the presence of the DGKi following T cell therapy administration may prevent or reduce exhaustion in T cells of the T cell therapy, thereby improving T cell anti-tumor activity and proliferative capacity following administration.

### Example 7: Continuous Compound 17 Dosing Regimen in Combination with Lower CAR T Cell Dose

The in vivo anti-tumor effects of a continuous Compound 17 dosing regimen in combination with a lower dose of CD19-directed CAR T cells were assessed. To produce the CD19-directed CAR T cells, T cells from a healthy human donor were engineered with a CD19-directed CAR.

For assessing in vivo anti-tumor effects, immunodeficient NSG mice were injected intravenously with 0.5×10⁶ Raji-rFluc cells. Seven days after tumor cell injection, mice received a single intravenous injection of 0.5×10⁶ or 1×10⁶ anti-CD19 CAR-expressing T cells (88.5% of total administered T cells) on Day 1 of the combination therapy or were left untreated. These doses of CAR-expressing T cells were not expected to achieve complete tumor rejection.

Following CAR-T cell administration, mice who received the lower dose of CAR T cells received 0.3 mg/kg of Compound 17 once daily on Days 1-30. Mice who received the higher dose of CAR T cells received a vehicle control or 0.3 mg/kg or 0.6 mg/kg of Compound 17 once daily on Days 1-30 (n=8 per group). All doses of Compound 17 were administered orally. For assessing tumor burden by bioluminescence imaging, mice received intraperitoneal injections of luciferin substrate resuspended in phosphate-buffered saline. Tumor burden and survival of treated mice were assessed until Day 50. In addition, flow cytometry for assessing circulating CAR T cell number and CD4 and CD8 expression was performed on Days 5, 12, 19, and 26. CAR expression was assessed using an anti-idiotypic (anti-ID) antibody specific for the CAR.

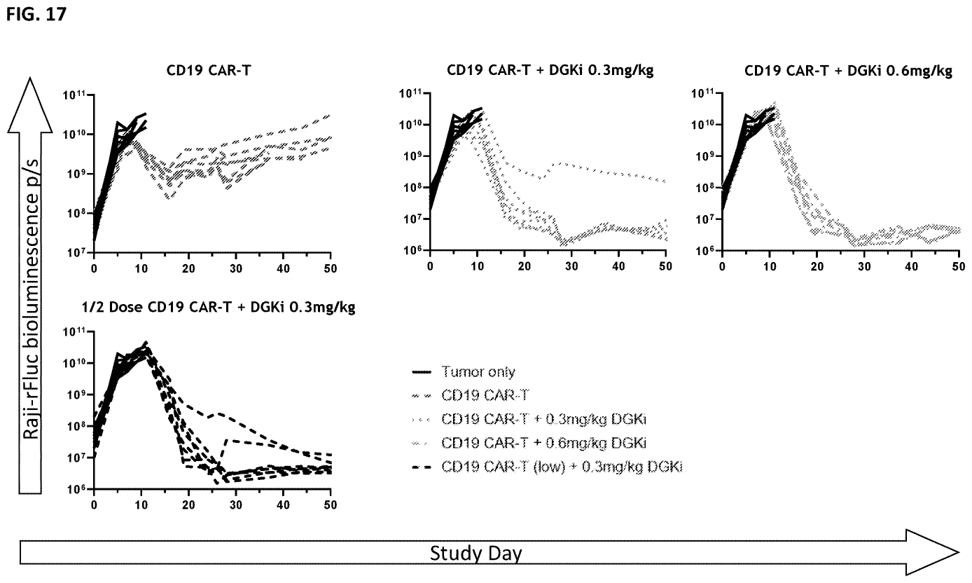

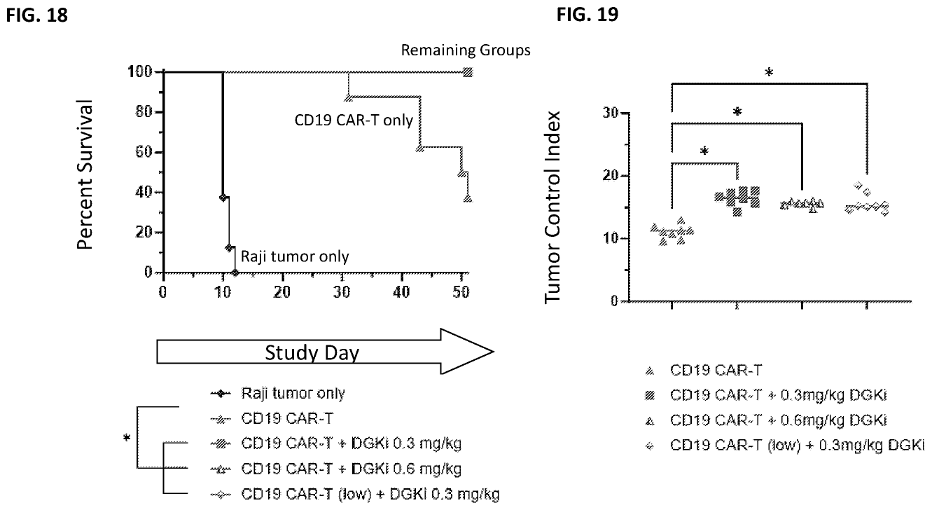

As shown in FIG. 17 and FIG. 18, and compared to administration of CAR T cells alone, administration of the CAR T cell combination with Compound 17 lowered tumor burden and improved survival in all groups, including the group that received the lower CAR T cell dose. As indicated by the tumor control index values shown in FIG. 19, administration of the CAR T cell combination with Compound 17 resulted in enhanced tumor suppression in all groups, relative to CAR T cell administration alone.

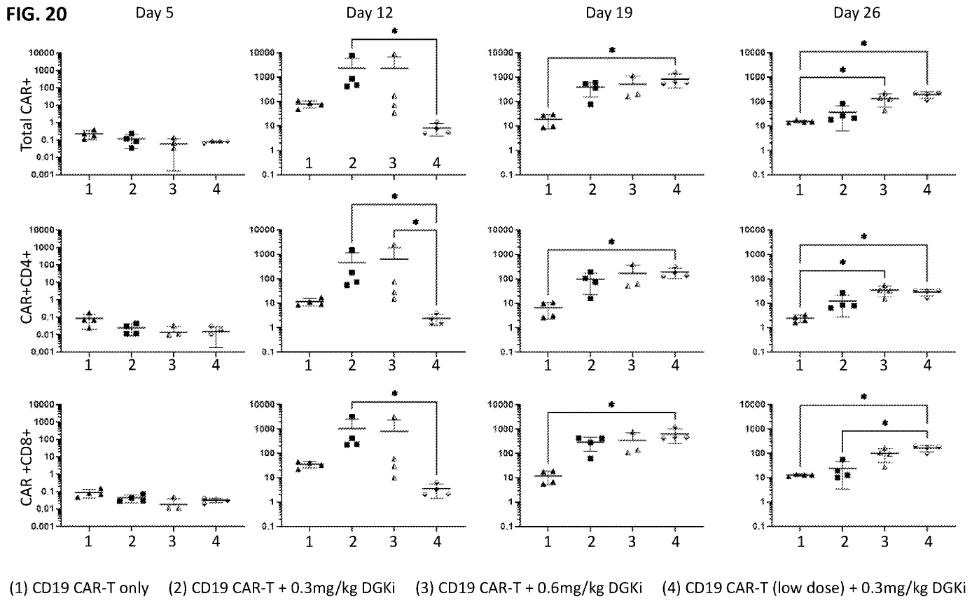

As shown in FIG. 20, administration of Compound 17 following CAR T cell administration increased CAR T cell expansion and/or persistence in all groups. CAR T cell expansion was delayed in the group that received the lower CAR T cell dose in combination with Compound 17, relative to the group that received the higher CAR T cell dose in combination with Compound 17. Circulating CAR T cell counts in the group that received the lower CAR T cell dose in combination with Compound 17 later exceeded circulating CAR T cell counts for the group that received the higher CAR T cell dose alone.

Together, these results show that reduced tumor burden and improved survival and CAR T cell expansion and/or persistence can be achieved by combination of CAR T cell administration with continuous DGKi administration, including when a lower dose of CAR T cells is administered.

### Example 8: Effects of Compound 17 Administration on Expansion and/or Persistence of CAR T Cells from Different Donors

The effects of Compound 17 administration on in vivo expansion and/or persistence of CAR T cells produced from different healthy donors were assessed. To produce the anti-CD19 CAR T cells, T cells from four healthy human donors were engineered with a CD19-directed CAR.

Immunodeficient NSG mice were injected intravenously with 0.5×10⁶ Raji-rFluc cells. Seven days after tumor cell injection, mice received a single intravenous injection of 1×10⁶ anti-CD19 CAR-expressing T cells (93.5% [Donor 1], 87.7% [Donor 2], 90.3% [Donor 3], or 97.6% [Donor 4] of total administered T cells) on Day 1 of the combination therapy or were left untreated. This dose of CAR-expressing T cells was not expected to achieve complete tumor rejection.

Following CAR-T cell administration, mice received a vehicle control or 0.3 mg/kg of Compound 17 once daily on Days 1-7 (n=4 per group). All doses of Compound 17 were administered orally. Flow cytometry for assessing circulating CAR T cell number was performed on Days 7 and 14. CAR expression was assessed using an anti-idiotypic (anti-ID) antibody specific for the CAR.

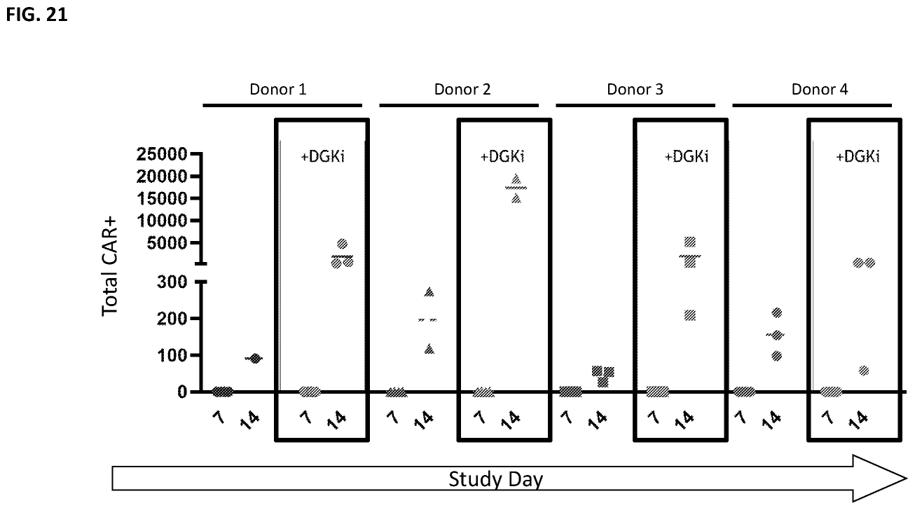

As shown in FIG. 21, CAR T cell expansion and/or persistence was increased in all mice that received Compound 17 administration, compared to CAR T cell administration alone. These results indicate that DGKi administration can improve expansion and/or persistence for CAR T cells produced from different donors, whose T cells can vary in differentiation state and the number of naive or naive-like T cells.

### Example 9: Comparison of CAR-T Cell Combination with Continuous Compound 17 Dosing Regimen to DGKα/ζ Single and Dual Knock-Out CAR T Cells

The in vivo anti-tumor effects of combining anti-CD19 CAR T cell therapy administration with a continuous Compound 17 dosing regimen were compared to the in vivo anti-tumor effects of administering DGKα single KO, DGKζ single KO, or DGKα/ζ dual KO anti-CD19 CAR T cells. To produce the anti-CD19 CAR T cells without DGK KO, T cells from a healthy human donor were engineered with a CD19-directed CAR. DGKα single KOs, DGKζ single KOs, and DGKα/ζ dual KOs were introduced via double-stranded breaks for indel generation at the locus, resulting in nonfunctional genes.

For assessing in vivo anti-tumor effects, immunodeficient NSG mice were injected intravenously with 0.5×10⁶ Raji-rFluc cells. Seven days after tumor cell injection, mice received a single intravenous injection of 1×10⁶ anti-CD19 CAR-expressing T cells (88.5% [without DGK KO], 87.8% [DGKα single KO], 88.2% [DGKζ single KO], or 86.6% [DGKα/ζ dual KO] of total administered T cells) on Day 1 of the combination therapy or were left untreated. This dose of CAR-expressing T cells was not expected to achieve complete tumor rejection.

Following CAR-T cell administration, mice who received the CAR T cells without DGK KO received a vehicle control or 0.3 mg/kg of Compound 17 once daily on Days 1-30 (n=8 per group). All doses of Compound 17 were administered orally. For assessing tumor burden by bioluminescence imaging, mice received intraperitoneal injections of luciferin substrate resuspended in phosphate-buffered saline. Tumor burden and survival of treated mice were assessed until Day 50. In addition, flow cytometry for assessing circulating CAR T cell number and CD4 and CD8 expression was performed on Days 5, 12, 19, and 26. CAR expression was assessed using an anti-idiotypic (anti-ID) antibody specific for the CAR.

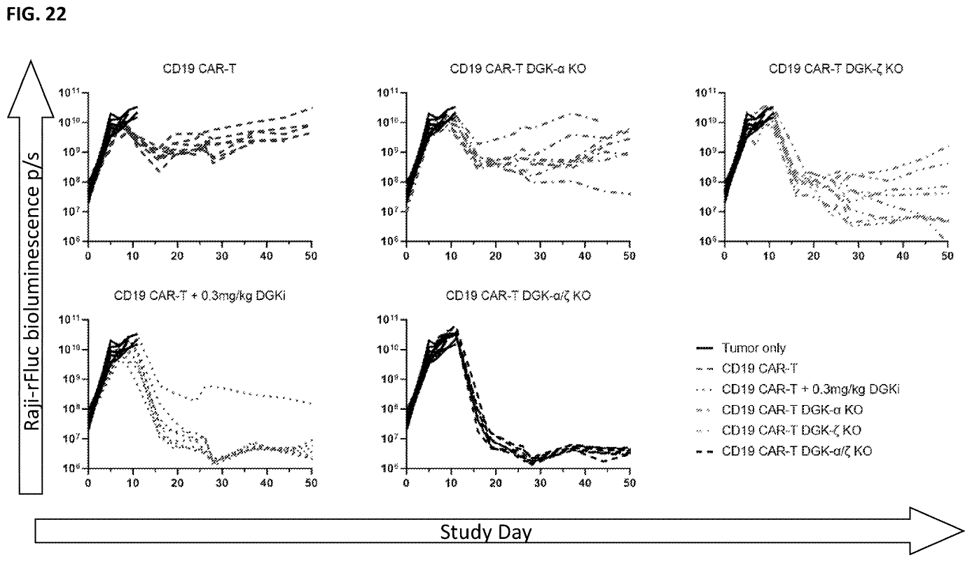

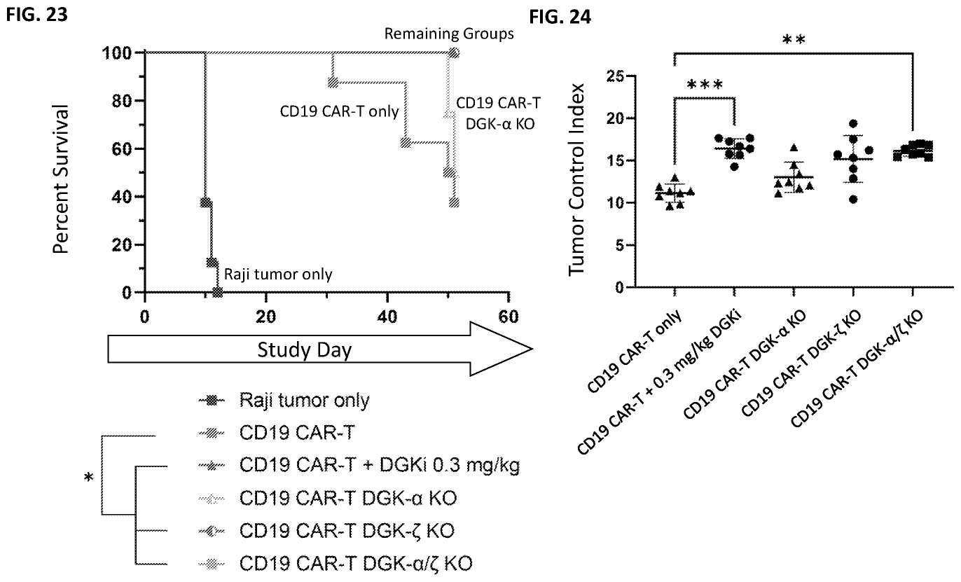

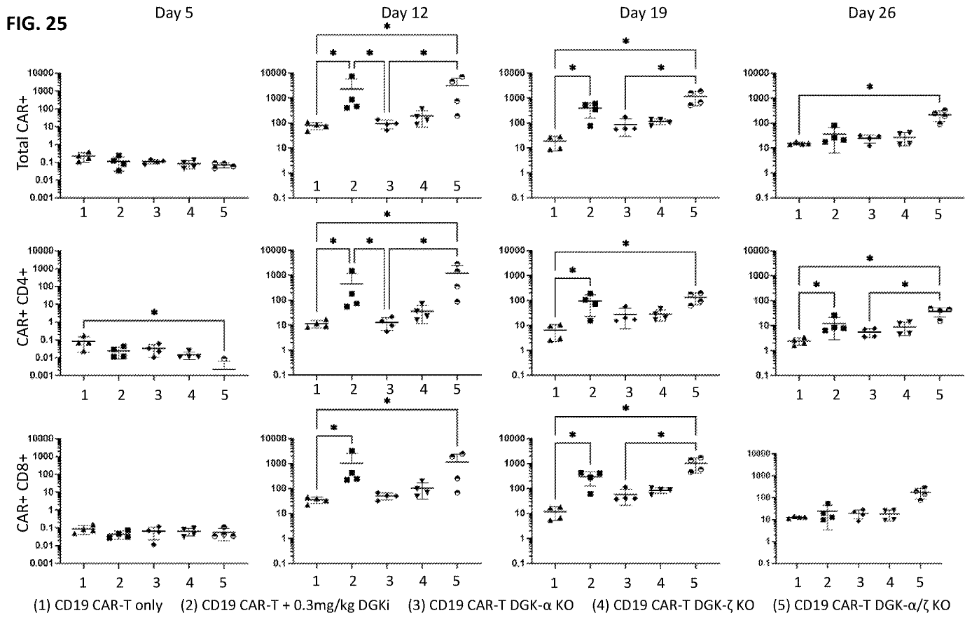

As shown in FIG. 22, tumor burden was reduced in the mice that received the CAR T cell combination with Compound 17 and the mice that received the DGKα/ζ dual KO CAR T cells. As shown in FIG. 23, improved survival was observed in mice that received the CAR T cell combination with Compound 17 and the DGKζ single KO or DGKα/dual KO CAR T cells, compared to mice that received CAR T cells without DGK KO alone. As indicated by the tumor control index values shown in FIG. 24, administration of the CAR T cell combination with Compound 17 and the DGKα/ζ dual KO CAR T cells showed improved anti-tumor efficacy, relative to CAR T cell administration without DGK KO alone. As shown in FIG. 25, improved CAR T cell expansion and/or persistence was observed in mice that received the CAR T cell combination with Compound 17 and the mice that received the DGKα/ζ dual KO CAR T cells.

Together, these results show that CAR T cell combination with continuous DGKi administration can lead to improvements in tumor burden, survival, and CAR T cell expansion and/or persistence that are comparable to those achieved with administration of DGKα/ζ dual KO CAR T cells.

### Example 10: CAR-T Cell Combination with Continuous Compound 17 Dosing Regimen in Second CD19-Expressing Tumor Model

The in vivo anti-tumor effects of a continuous Compound 17 dosing regimen in combination with anti-CD19 CAR T cells were assessed in a second CD19-expressing tumor model. To produce the anti-CD19 CAR T cells, T cells from a healthy human donor were engineered with a CD19-directed CAR.

For assessing in vivo anti-tumor effects, immunodeficient NSG mice were injected intravenously with 0.5×10⁶ Nalm6 leukemia tumor cells (an immortalized human B cell precursor leukemia tumor cell line that expresses CD19) that were transduced with red-shifted firefly luciferase (Nalm6-rFluc cells). Compared to Raji cells, Nalm6 cells do not express cell surface costimulatory molecules, such that CAR T cells can be less likely to be activated in the presence of Nalm6 cells, compared to Raji cells. Four days after tumor cell injection, mice received a single intravenous injection of 1×10⁶ anti-CD19 CAR-expressing T cells (81.8% of total administered T cells) on Day 1 of the combination therapy or were left untreated. This dose of CAR-expressing T cells was not expected to achieve complete tumor rejection.

Following CAR-T cell administration, mice received a vehicle control or 0.3 mg/kg of Compound 17 once daily on Days 1-30. Other mice received 0.3 mg/kg or 0.6 mg/kg of Compound 17 once daily on Days 1-30, but without prior CAR-T cell administration (n=8 per group). All doses of Compound 17 were administered orally. For assessing tumor burden by bioluminescence imaging, mice received intraperitoneal injections of luciferin substrate resuspended in phosphate-buffered saline. Tumor burden and survival of treated mice were assessed until Day 50. In addition, flow cytometry for assessing circulating CAR T cell number and CD4 and CD8 expression was performed on Days 8, 16, 23, and 30. CAR expression was assessed using an anti-idiotypic (anti-ID) antibody specific for the CAR.

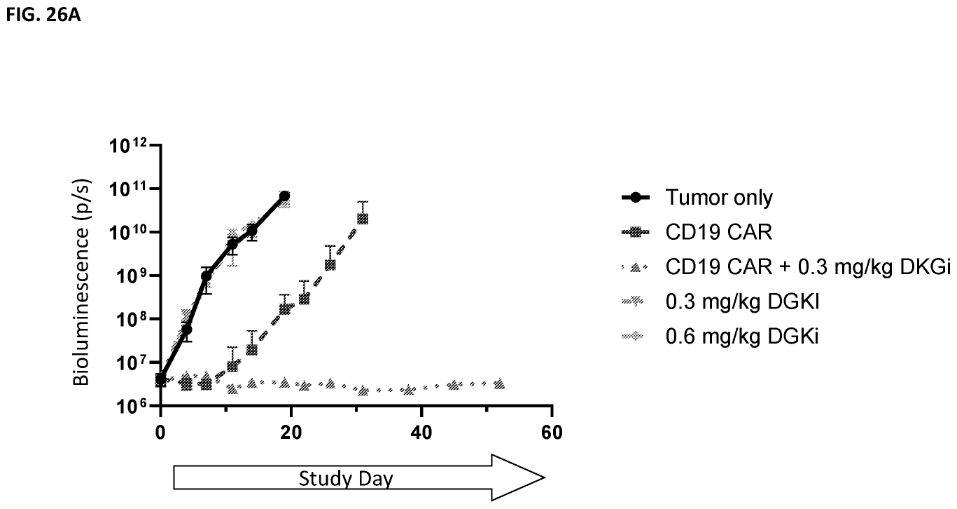

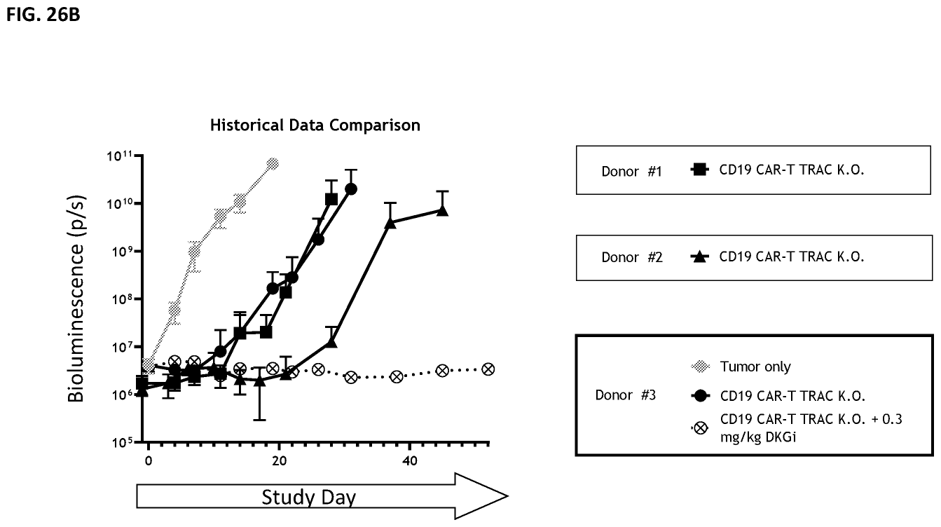

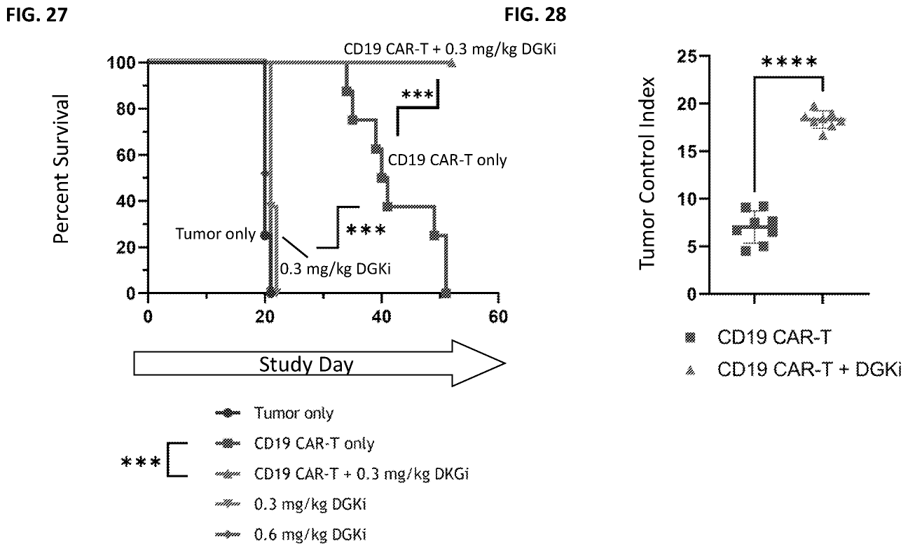

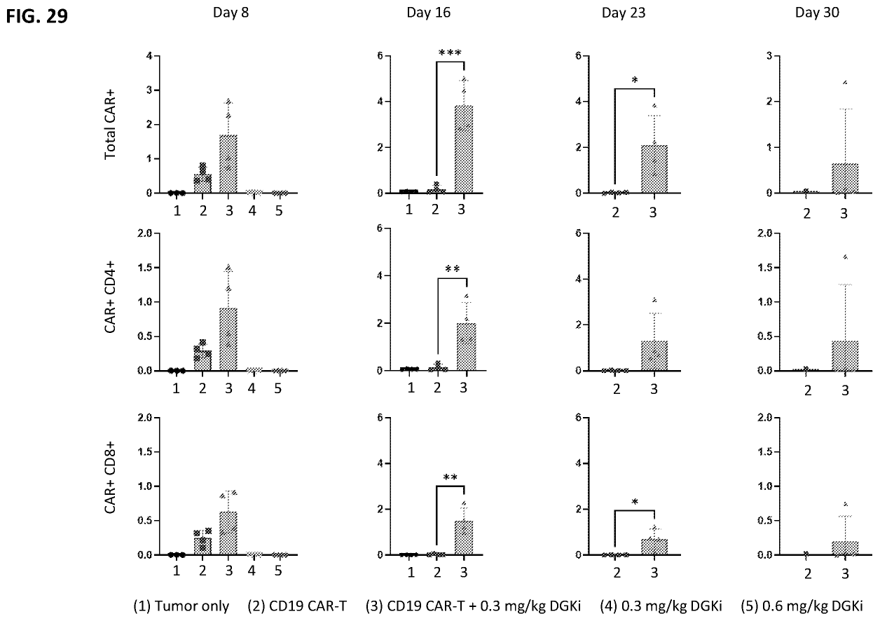

As shown in FIG. 26A, FIG. 26B, and FIG. 27, administration of the CAR T cell combination with Compound 17 lowered tumor burden and improved survival, compared to CAR T cell administration or Compound 17 administration alone. No single-agent effects of Compound 17 were observed. FIG. 26B also shows historical tumor burden data from separate previous experiments with Nalm6-rFluc injected mice who had also received 1×10⁶ of anti-CD19 CAR-expressing T cells produced from one of two healthy human donors (Donor 1 and Donor 2). Mice in the separate previous experiments did not receive Compound 17 administration. As shown in FIG. 26B, mice in the present experiment that received the CAR T cell combination with Compound 17 also had lower tumor burden than the mice in the separate previous experiments. As indicated by the tumor control index values shown in FIG. 28, CAR T cell combination with Compound 17 resulted in enhanced tumor suppression relative to CAR T cell administration alone. As shown in FIG. 29, administration of Compound 17 following CAR T cell administration increased CAR T cell expansion and/or persistence.

Together, these results show in a second CD19-expressing tumor model that reduced tumor burden and improved survival and CAR T cell expansion and/or persistence can be achieved by combination of CAR T cell administration with continuous DGKi administration.

### Example 11: Rₑ-Challenge with Tumor Cells Following CAR-T Cell Combination with Continuous Compound 17 Dosing Regimen

Mice in Example 10 who received the CAR T cell combination with continuous Compound 17 dosing regimen were re-challenged by intravenous injection of 5×10⁶ Nalm6-rFluc cells on Day 71. For comparison, 5×10⁶ Nalm6-rFluc cells were intravenously injected into naive mice that had not previously been injected with or administered Nalm6-rFluc cells, CAR T cells, or Compound 17 (n=4 per group). For assessing tumor burden by bioluminescence imaging, mice received intraperitoneal injections of luciferin substrate resuspended in phosphate-buffered saline. Tumor burden was assessed for 10 days following re-challenge. Survival of treated mice was assessed until 30 days post re-challenge.

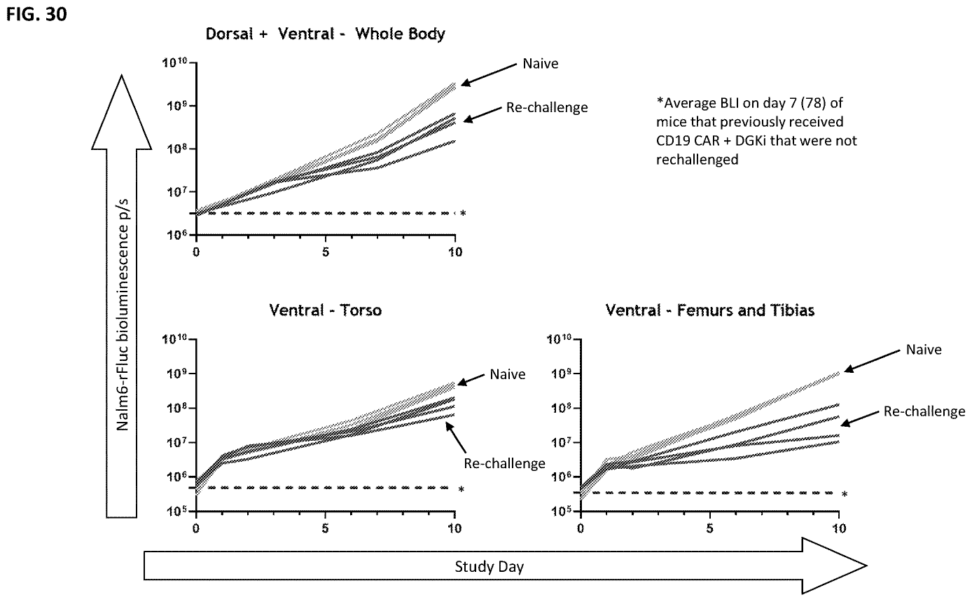

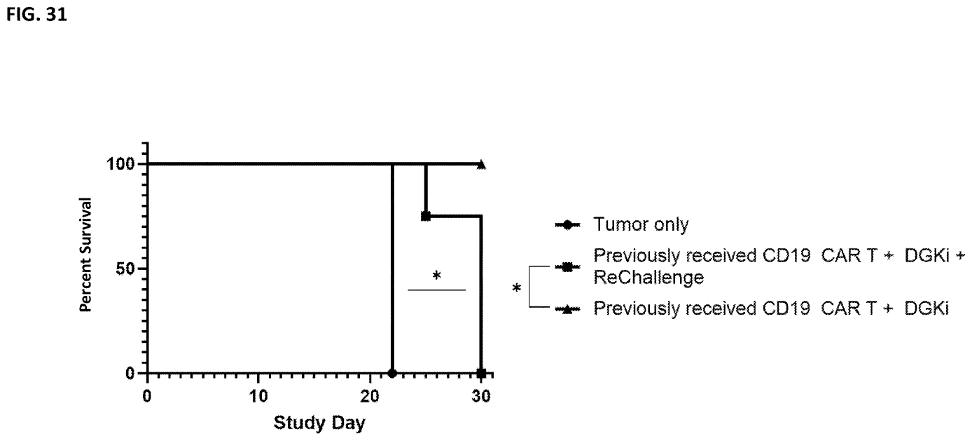

As shown in FIG. 30, mice that were re-challenged with tumor cells following administration of the CAR T cell combination with Compound 17 had delayed tumor growth, in particular within the long bones, compared to the naive mice. A survival benefit was also observed for the re-challenged mice, as shown in FIG. 31. These results indicate that prior exposure to the CAR T cell combination with continuous DGKi administration can have long-term benefits for protecting against later tumor cell re-challenge.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| SEQ | | |
|---|---|---|
| ID | | |
| NO. | SEQUENCE | DESCRIPTION |
| 1 | ESKYGPPCPPCP | spacer |
| | | (IgG4hinge) (aa) |
| | | Homo sapiens |
| 2 | gaatctaagtacggaccgccctgccccccttgccct | spacer |
| | | (IgG4hinge) (nt) |
| | | Homo sapiens |
| 3 | ESKYGPPCPPCPGQPREPQVYTLPPSQEEMTKNQVSLTCLVK | Hinge-CH3 |
| | GFYPSDIAVEWESNGQPENNYKTTPPVLDSDGSFFLYSRLTV | spacer |
| | DKSRWQEGNVFSCSVMHEALHNHYTQKSLSLSLGK | Homo sapiens |
| 4 | ESKYGPPCPPCPAPEFLGGPSVFLFPPKPKDTLMISRTPEVTCV | Hinge-CH2-CH3 |
| | VVDVSQEDPEVQFNWYVDGVEVHNAKTKPREEQFNSTYRV | spacer |
| | VSVLTVLHQDWLNGKEYKCKVSNKGLPSSIEKTISKAKGQP | Homo sapiens |
| | REPQVYTLPPSQEEMTKNQVSLTCLVKGFYPSDIAVEWESNG | |
| | QPENNYKTTPPVLDSDGSFFLYSRLTVDKSRWQEGNVFSCSV | |
| | MHEALHNHYTQKSLSLSLGK | |
| 5 | RWPESPKAQASSVPTAQPQAEGSLAKATTAPATTRNTGRGG | IgD-hinge-Fc |
| | EEKKKEKEKEEQEERETKTPECPSHTQPLGVYLLTPAVQDL | Homo sapiens |
| | WLRDKATFTCFVVGSDLKDAHLTWEVAGKVPTGGVEEGLL | |
| | ERHSNGSQSQHSRLTLPRSLWNAGTSVTCTLNHPSLPPQRLM | |
| | ALREPAAQAPVKLSLNLLASSDPPEAASWLLCEVSGFSPPNIL | |
| | LMWLEDQREVNTSGFAPARPPPQPGSTTFWAWSVLRVPAPP | |
| | SPQPATYTCVVSHEDSRTLLNASRSLEVSYVTDH | |
| 6 | LEGGGEGRGSLLTCGDVEENPGPR | T2A |
| | | artificial |
| 7 | RKVCNGIGIGEFKDSLSINATNIKHFKNCTSISGDLHILPVAFR | tEGFR |
| | GDSFTHTPPLDPQELDILKTVKEITGFLLIQAWPENRTDLHAF | artificial |
| | ENLEIIRGRTKQHGQFSLAVVSLNITSLGLRSLKEISDGDVIIS | |
| | GNKNLCYANTINWKKLFGTSGQKTKIISNRGENSCKATGQV | |
| | CHALCSPEGCWGPEPRDCVSCRNVSRGRECVDKCNLLEGEP | |
| | REFVENSECIQCHPECLPQAMNITCTGRGPDNCIQCAHYIDGP | |
| | HCVKTCPAGVMGENNTLVWKYADAGHVCHLCHPNCTYGC | |
| | TGPGLEGCPTNGPKIPSIATGMVGALLLLLVVALGIGLFM | |
| 8 | FWVLVVVGGVLACYSLLVTVAFIIFWV | CD28 (amino |
| | | acids 153-179 of |
| | | Accession No. |
| | | P10747) |
| | | Homo sapiens |
| 9 | IEVMYPPPYLDNEKSNGTIIHVKGKHLCPSPLFPGPSKPFWVL | CD28 (amino |
| | VVVGGVLACYSLLVTVAFIIFWV | acids 114-179 of |
| | | Accession No. |
| | | P10747) |
| | | Homo sapiens |
| 10 | RSKRSRLLHSDYMNMTPRRPGPTRKHYQPYAPPRDFAAYRS | CD28 (amino |
| | | acids 180-220 of |
| | | P10747) |
| | | Homo sapiens |
| 11 | RSKRSRGGHSDYMNMTPRRPGPTRKHYQPYAPPRDFAAYRS | CD28 (LL to GG) |
| | | Homo sapiens |
| 12 | KRGRKKLLYIFKQPFMRPVQTTQEEDGCSCRFPEEEEGGCEL | 4-1BB (amino |
| | | acids 214-255 of |
| | | Q07011.1) |
| | | Homo sapiens |
| 13 | RVKFSRSADAPAYQQGQNQLYNELNLGRREEYDVLDKRRG | CD3 zeta |
| | RDPEMGGKPRRKNPQEGLYNELQKDKMAEAYSEIGMKGER | Homo sapiens |
| | RRGKGHDGLYQGLSTATKDTYDALHMQALPPR | |
| 14 | RVKFSRSAEPPAYQQGQNQLYNELNLGRREEYDVLDKRRGR | CD3 zeta |
| | DPEMGGKPRRKNPQEGLYNELQKDKMAEAYSEIGMKGERR | Homo sapiens |
| | RGKGHDGLYQGLSTATKDTYDALHMQALPPR | |
| 15 | RVKFSRSADAPAYKQGQNQLYNELNLGRREEYDVLDKRRG | CD3 zeta |
| | RDPEMGGKPRRKNPQEGLYNELQKDKMAEAYSEIGMKGER | Homo sapiens |
| | RRGKGHDGLYQGLSTATKDTYDALHMQALPPR | |
| 16 | PGGG-(SGGGG)₅-P wherein P is proline, G is glycine and S is | Linker |
| | serine | |
| 17 | GSADDAKKDAAKKDGKS | Linker |
| 18 | MLQMAGQCSQNEYFDSLLHACIPCQLRCSSNTPPLTCQRYC | Extracellular |
| | NASVTNSVKGTNA | domain of human |
| | | BCMA |
| | | (GenBank No. |
| | | NP_001183.2) |
| 19 | GGGGS | Linker sequence |
| 20 | PKSSDKTHTCPPCPAPEAEGAPSVFLFPPKPKDTLMISRTPEV | Modified Human |
| | TCVVVDVSHEDPEVKFNWYVDGVEVHNAKTKPREEQYNST | IgG1 Fc |
| | YRVVSVLTVLHQDWLNGKEYKCKVSNKALPSSIEKTISKAK | |
| | GQPREPQVYTLPPSRDELTKNQVSLTCLVKGFYPSDIAVEWE | |
| | SNGQPENNYKTTPPVLDSDGSFFLYSKLTVDKSRWQQGNVF | |
| | SCSVMHEALHNHYTQKSLSLSPGK | |
| 21 | MPLLLLLPLLWAGALA | CD33 Signal |
| | | peptide |
| 22 | MPLLLLLPLLWAGALAMLQMAGQCSQNEYFDSLLHACIPCQ | BCMA-Fc |
| | LRCSSNTPPLTCQRYCNASVTNSVKGTNAGGGGSPKSSDKT | construct |
| | HTCPPCPAPEAEGAPSVFLFPPKPKDTLMISRTPEVTCVVVDV | |
| | SHEDPEVKFNWYVDGVEVHNAKTKPREEQYNSTYRVVSVL | |
| | TVLHQDWLNGKEYKCKVSNKALPSSIEKTISKAKGQPREPQ | |
| | VYTLPPSRDELTKNQVSLTCLVKGFYPSDIAVEWESNGQPEN | |
| | NYKTTPPVLDSDGSFFLYSKLTVDKSRWQQGNVFSCSVMHE | |
| | ALHNHYTQKSLSLSPGK | |
| 23 | EGRGSLLTCGDVEENPGP | T2A |
| 24 | GSGATNFSLLKQAGDVEENPGP | P2A |
| 25 | ATNFSLLKQAGDVEENPGP | P2A |
| 26 | QCTNYALLKLAGDVESNPGP | E2A |
| 27 | VKQTLNFDLLKLAGDVESNPGP | F2A |
| 28 | RKVCNGIGIGEFKDSLSINATNIKHFKNCTSISGDLHILPVAFR | tEGFR |
| | GDSFTHTPPLDPQELDILKTVKEITGFLLIQAWPENRTDLHAF | artificial |
| | ENLEIIRGRTKQHGQFSLAVVSLNITSLGLRSLKEISDGDVIIS | |
| | GNKNLCYANTINWKKLFGTSGQKTKIISNRGENSCKATGQV | |
| | CHALCSPEGCWGPEPRDCVSCRNVSRGRECVDKCNLLEGEP | |
| | REFVENSECIQCHPECLPQAMNITCTGRGPDNCIQCAHYIDGP | |
| | HCVKTCPAGVMGENNTLVWKYADAGHVCHLCHPNCTYGC | |
| | TGPGLEGCPTNGPKIPSIATGMVGALLLLLVVALGIGLFM | |
| 29 | ESKYGPPCPPCPAPPVAGPSVFLFPPKPKDTLMISRTPEVTCV | Hinge-CH2-CH3 |
| | VVDVSQEDPEVQFNWYVDGVEVHNAKTKPREEQFQSTYRV | spacer |
| | VSVLTVLHQDWLNGKEYKCKVSNKGLPSSIEKTISKAKGQP | Homo sapiens |
| | REPQVYTLPPSQEEMTKNQVSLTCLVKGFYPSDIAVEWESNG | |
| | QPENNYKTTPPVLDSDGSFFLYSRLTVDKSRWQEGNVFSCSV | |
| | MHEALHNHYTQKSLSLSLGK | |
| 30 | QIQLVQSGPELKKPGETVKISCKASGYTFTDYSINWVKRAPG | Variable heavy |
| | KGLKWMGWINTETREPAYAYDFRGRFAFSLETSASTAYLQI | (VH) Anti- |
| | NNLKYEDTATYFCALDYSYAMDYWGQGTSVTVSS | BCMA |
| 31 | DIVLTQSPPSLAMSLGKRATISCRASESVTILGSHLIHWYQQK | Variable light |
| | PGQPPTLLIQLASNVQTGVPARFSGSGSRTDFTLTIDPVEEDD | (VL) Anti- |
| | VAVYYCLQSRTIPRTFGGGTKLEIK | BCMA |
| 32 | QIQLVQSGPDLKKPGETVKLSCKASGYTFTNFGMNWVKQAP | Variable heavy |
| | GKGFKWMAWINTYTGESYFADDFKGRFAFSVETSATTAYLQ | (VH) Anti- |
| | INNLKTEDTATYFCARGEIYYGYDGGFAYWGQGTLVTVSA | BCMA |
| 33 | DVVMTQSHRFMSTSVGDRVSITCRASQDVNTAVSWYQQKP | Variable light |
| | GQSPKLLIFSASYRYTGVPDRFTGSGSGADFTLTISSVQAEDL | (VL) Anti- |
| | AVYYCQQHYSTPWTFGGGTKLDIK | BCMA |
| 34 | EVQLVQSGAEVKKPGESLKISCKGSGYSFTSYWIGWVRQMP | Variable heavy |
| | GKGLEWMGIIYPGDSDTRYSPSFQGHVTISADKSISTAYLQW | (VH) Anti- |
| | SSLKASDTAMYYCARYSGSFDNWGQGTLVTVSS | BCMA |
| 35 | SYELTQPPSASGTPGQRVTMSCSGTSSNIGSHSVNWYQQLPG | Variable light |
| | TAPKLLIYTNNQRPSGVPDRFSGSKSGTSASLAISGLQSEDEA | (VL) Anti- |
| | DYYCAAWDGSLNGLVFGGGTKLTVLG | BCMA |
| 36 | EVQLVQSGAEMKKPGASLKLSCKASGYTFIDYYVYWMRQA | Variable heavy |
| | PGQGLESMGWINPNSGGTNYAQKFQGRVTMTRDTSISTAYM | (VH) Anti- |
| | ELSRLRSDDTAMYYCARSQRDGYMDYWGQGTLVTVSS | BCMA |
| 37 | QSALTQPASVSASPGQSIAISCTGTSSDVGWYQQHPGKAPKL | Variable light |
| | MIYEDSKRPSGVSNRFSGSKSGNTASLTISGLQAEDEADYYC | (VL) Anti- |
| | SSNTRSSTLVFGGGTKLTVLG | BCMA |
| 38 | EVQLVQSGAEVKKPGSSVKVSCKASGGTFSSYAISWVRQAP | Variable heavy |
| | GQGLEWMGRIIPILGIANYAQKFQGRVTMTEDTSTDTAYME | (VH) Anti- |
| | LSSLRSEDTAVYYCARSGYSKSIVSYMDYWGQGTLVTVSS | BCMA |
| 39 | LPVLTQPPSTSGTPGQRVTVSCSGSSSNIGSNVVFWYQQLPGT | Variable light |
| | APKLVIYRNNQRPSGVPDRFSVSKSGTSASLAISGLRSEDEAD | (VL) Anti- |
| | YYCAAWDDSLSGYVFGTGTKVTVLG | BCMA |
| 40 | QVQLVQSGAEVKKPGSSVKVSCKASGGTFSSYAISWVRQAP | Variable heavy |
| | GQGLEWMGRIIPILGTANYAQKFQGRVTITADESTSTAYMEL | (VH) Anti- |
| | SSLRSEDTAVYYCARSGYGSYRWEDSWGQGTLVTVSS | BCMA |
| 41 | QAVLTQPPSASGTPGQRVTISCSGSSSNIGSNYVFWYQQLPGT | Variable light |
| | APKLLIYSNNQRPSGVPDRFSGSKSGTSASLAISGLRSEDEAD | (VL) Anti- |
| | YYCAAWDDSLSASYVFGTGTKVTVLG | BCMA |
| 42 | QVQLVQSGAEVKKPGASVKVSCKASGYTFTDYYMHWVRQ | Variable heavy |
| | APGQRLEWMGWINPNSGGTNYAQKFQDRITVTRDTSSNTGY | (VH) Anti- |
| | MELTRLRSDDTAVYYCARSPYSGVLDKWGQGTLVTVSS | BCMA |
| 43 | QSVLTQPPSVSGAPGQRVTISCTGSSSNIGAGFDVHWYQQLP | Variable light |
| | GTAPKLLIYGNSNRPSGVPDRFSGSKSGTSASLAITGLQAEDE | (VL) Anti- |
| | ADYYCQSYDSSLSGYVFGTGTKVTVLG | BCMA |
| 44 | DYGVS | FMC63 CDR H1 |
| 45 | VIWGSETTYYNSALKS | FMC63 CDR H2 |
| 46 | YAMDYWG | FMC63 CDR H3 |
| 47 | HYYYGGSYAMDY | FMC63 HC- |
| | | CDR3 |
| 48 | RASQDISKYLN | FMC63 CDR L1 |
| 49 | SRLHSGV | FMC63 CDR L2 |
| 50 | HTSRLHS | FMC63 LC- |
| | | CDR2 |
| 51 | GNTLPYTFG | FMC63 CDR L3 |
| 52 | QQGNTLPYT | FMC63 LC- |
| | | CDR3 |
| 53 | EVKLQESGPGLVAPSQSLSVTCTVSGVSLPDYGVSWIRQPPR | FMC63 VH |
| | KGLEWLGVIWGSETTYYNSALKSRLTIIKDNSKSQVFLKMNS | |
| | LQTDDTAIYYCAKHYYYGGSYAMDYWGQGTSVTVSS | |
| 54 | DIQMTQTTSSLSASLGDRVTISCRASQDISKYLNWYQQKPDG | FMC63 VL |
| | TVKLLIYHTSRLHSGVPSRFSGSGSGTDYSLTISNLEQEDIATY | |
| | FCQQGNTLPYTFGGGTKLEIT | |
| 55 | DIQMTQTTSSLSASLGDRVTISCRASQDISKYLNWYQQKPDG | FMC63 scFv |
| | TVKLLIYHTSRLHSGVPSRFSGSGSGTDYSLTISNLEQEDIATY | |
| | FCQQGNTLPYTFGGGTKLEITGSTSGSGKPGSGEGSTKGEVK | |
| | LQESGPGLVAPSQSLSVTCTVSGVSLPDYGVSWIRQPPRKGL | |
| | EWLGVIWGSETTYYNSALKSRLTIIKDNSKSQVFLKMNSLQT | |
| | DDTAIYYCAKHYYYGGSYAMDYWGQGTSVTVSS | |
| 56 | KASQNVGTNVA | SJ25C1 CDR L1 |
| 57 | SATYRNS | SJ25C1 CDR L2 |
| 58 | QQYNRYPYT | SJ25C1 CDR L3 |
| 59 | SYWMN | SJ25C1 CDR H1 |
| 60 | QIYPGDGDTNYNGKFKG | SJ25C1 CDR H2 |
| 61 | KTISSVVDFYFDY | SJ25C1 CDR H3 |
| 62 | EVKLQQSGAELVRPGSSVKISCKASGYAFSSYWMNWVKQRP | SJ25C1 VH |
| | GQGLEWIGQIYPGDGDTNYNGKFKGQATLTADKSSSTAYM | |
| | QLSGLTSEDSAVYFCARKTISSVVDFYFDYWGQGTTVTVSS | |
| 63 | DIELTQSPKFMSTSVGDRVSVTCKASQNVGTNVAWYQQKPG | SJ25C1 VL |
| | QSPKPLIYSATYRNSGVPDRFTGSGSGTDFTLTITNVQSKDLA | |
| | DYFCQQYNRYPYTSGGGTKLEIKR | |
| 64 | GGGGSGGGGSGGGGS | Linker |
| 65 | EVKLQQSGAELVRPGSSVKISCKASGYAFSSYWMNWVKQRP | SJ25C1 scFv |
| | GQGLEWIGQIYPGDGDTNYNGKFKGQATLTADKSSSTAYM | |
| | QLSGLTSEDSAVYFCARKTISSVVDFYFDYWGQGTTVTVSSG | |
| | GGGSGGGGSGGGGSDIELTQSPKFMSTSVGDRVSVTCKASQ | |
| | NVGTNVAWYQQKPGQSPKPLIYSATYRNSGVPDRFTGSGSG | |
| | TDFTLTITNVQSKDLADYFCQQYNRYPYTSGGGTKLEIKR | |
| 66 | HYYYGGSYAMDY | FMC63 HC- |
| | | CDR3 |
| 67 | HTSRLHS | FMC63 LC- |
| | | CDR2 |
| 68 | QQGNTLPYT | FMC63 LC- |
| | | CDR3 |
| 69 | gacatccagatgacccagaccacctccagcctgagcgccagcctgggcgaccgggtgaccat | Sequence |
| | cagctgccgggccagccaggacatcagcaagtacctgaactggtatcagcagaagcccgacg | encoding scFv |
| | gcaccgtcaagctgctgatctaccacaccagccggctgcacagcggcgtgcccagccggttta | |
| | gcggcagcggctccggcaccgactacagcctgaccatctccaacctggaacaggaagatatcg | |
| | ccacctacttttgccagcagggcaacacactgccctacacctttggcggcggaacaaagctgga | |
| | aatcaccggcagcacctccggcagcggcaagcctggcagcggcgagggcagcaccaaggg | |
| | cgaggtgaagctgcaggaaagcggccctggcctggtggcccccagccagagcctgagcgtg | |
| | acctgcaccgtgagcggcgtgagcctgcccgactacggcgtgagctggatccggcagccccc | |
| | caggaagggcctggaatggctgggcgtgatctggggcagcgagaccacctactacaacagcg | |
| | ccctgaagagccggctgaccatcatcaaggacaacagcaagagccaggtgttcctgaagatga | |
| | acagcctgcagaccgacgacaccgccatctactactgcgccaagcactactactacggcggca | |
| | gctacgccatggactactggggccagggcaccagcgtgaccgtgagcagc | |
| 70 | GSTSGSGKPGSGEGSTKG | Linker |
| 71 | GGGS | Linker |
| 72 | GGGGSGGGGSGGGGS | Linker |
| 73 | GSTSGSGKPGSGEGSTKG | Linker |
| 74 | SRGGGGSGGGGSGGGGSLEMA | Linker |
| 75 | MALPVTALLLPLALLLHAARP | CD8a signal |
| | | peptide |
| 76 | METDTLLLWVLLLWVPGSTG | Signal peptide |
| 77 | EVQLLESGGGLVQPGGSLRLSCAASGFTFSSYAMSWVRQAP | Variable heavy |
| | GKGLEWVSAISGSGGSTYYADSVKGRFTISRDNSKNTLYLQ | (VH) Anti- |
| | MNSLRAEDTAVYYCARAEMGAVFDIWGQGTMVTVSS | BCMA |
| 78 | EIVLTQSPATLSLSPGERATLSCRASQSVSRYLAWYQQKPGQ | Variable light |
| | APRLLIYDASNRATGIPARFSGSGSGTDFTLTISSLEPEDFAVY | (VL) Anti- |
| | YCQQRISWPFTFGGGTKVEIK | BCMA |
| 79 | QVQLVESGGGVVQPGRSLRLSCAASGFTFSSYGMHWVRQAP | Variable heavy |
| | GKGLEWVAVISYDGSNKYYADSVKGRFTISRDNSKNTLYLQ | (VH) Anti- |
| | MNSLRAEDTAVYYCARDGTYLGGLWYFDLWGRGTLVTVSS | BCMA |
| 80 | DIVMTQSPLSLPVTPGEPASISCRSSQSLLHSNGYNYLDWYLQ | Variable light |
| | KPGQSPQLLIYLGSNRASGVPDRFSGSGSGTDFTLKISRVEAE | (VL) Anti- |
| | DVGVYYCMQGLGLPLTFGGGTKVEIK | BCMA |
| 81 | QVQLVQSGAEVKKPGASVKVSCKASGYTFTSYYMHWVRQA | Variable heavy |
| | PGQGLEWMGIINPGGGSTSYAQKFQGRVTMTRDTSTSTVYM | (VH) Anti- |
| | ELSSLRSEDTAVYYCARESWPMDVWGQGTTVTVSS | BCMA |
| 82 | EIVMTQSPATLSVSPGERATLSCRASQSVSSNLAWYQQKPGQ | Variable light |
| | APRLLIYGASTRATGIPARFSGSGSGTEFTLTISSLQSEDFAVY | (VL) Anti- |
| | YCQQYAAYPTFGGGTKVEIK | BCMA |
| 83 | QLQLQESGPGLVKPSETLSLTCTVSGGSISSSSYYWGWIRQPP | Variable heavy |
| | GKGLEWIGSISYSGSTYYNPSLKSRVTISVDTSKNQFSLKLSS | (VH) Anti- |
| | VTAADTAVYYCARGRGYATSLAFDIWGQGTMVTVSS | BCMA |
| 84 | EIVLTQSPATLSLSPGERATLSCRASQSVSSYLAWYQQKPGQ | Variable light |
| | APRLLIYDASNRATGIPARFSGSGSGTDFTLTISSLEPEDFAVY | (VL) Anti- |
| | YCQQRHVWPPTFGGGTKVEIK | BCMA |
| 85 | EVQLVESGGGLVQPGGSLRLSCAASGFTFSSYSMNWVRQAP | Variable heavy |
| | GKGLEWVSTISSSSSTIYYADSVKGRFTISRDNAKNSLYLQM | (VH) Anti- |
| | NSLRAEDTAVYYCARGSQEHLIFDYWGQGTLVTVSS | BCMA |
| 86 | EIVLTQSPATLSLSPGERATLSCRASQSVSRYLAWYQQKPGQ | Variable light |
| | APRLLIYDASNRATGIPARFSGSGSGTDFTLTISSLEPEDFAVY | (VL) Anti- |
| | YCQQRFYYPWTFGGGTKVEIK | BCMA |
| 87 | QVQLVESGGGVVQPGRSLRLSCAASGFTFSSYGMHWVRQAP | Variable heavy |
| | GKGLEWVAVISYDGSNKYYADSVKGRFTISRDNSKNTLYLQ | (VH) Anti- |
| | MNSLRAEDTAVYYCARTDFWSGSPPGLDYWGQGTLVTVSS | BCMA |
| 88 | DIQLTQSPSSVSASVGDRVTITCRASQGISSWLAWYQQKPGK | Variable light |
| | APKLLIYGASSLQSGVPSRFSGSGSGTDFTLTISSLQPEDFATY | (VL) Anti- |
| | YCQQIYTFPFTFGGGTKVEIK | BCMA |
| 89 | QVQLVQSGAEVKKPGSSVKVSCKASGGTFSSYAISWVRQAP | Variable heavy |
| | GQGLEWMGGIIPIFGTANYAQKFQGRVTITADESTSTAYMEL | (VH) Anti- |
| | SSLRSEDTAVYYCARTPEYSSSIWHYYYGMDVWGQGTTVT | BCMA |
| | VSS | |
| 90 | DIVMTQSPDSLAVSLGERATINCKSSQSVLYSSNNKNYLAWY | Variable light |
| | QQKPGQPPKLLIYWASTRESGVPDRFSGSGSGTDFTLTISSLQ | (VL) Anti- |
| | AEDVAVYYCQQFAHTPFTFGGGTKVEIK | BCMA |
| 91 | QVQLVESGGGVVQPGRSLRLSCAASGFTFSSYGMHWVRQAP | Variable heavy |
| | GKGLEWVAVISYDGSNKYYADSVKGRFTISRDNSKNTLYLQ | (VH) Anti- |
| | MNSLRAEDTAVYYCVKGPLQEPPYDYGMDVWGQGTTVTV | BCMA |
| | SS | |
| 92 | EIVMTQSPATLSVSPGERATLSCRASQSVSSNLAWYQQKPGQ | Variable light |
| | APRLLIYSASTRATGIPARFSGSGSGTEFTLTISSLQSEDFAVY | (VL) Anti- |
| | YCQQHHVWPLTFGGGTKVEIK | BCMA |
| 93 | QVQLVQSGAEVKKPGSSVKVSCKASGGTFSSYAISWVRQAP | Variable heavy |
| | GQGLEWMGRIIPILGIANYAQKFQGRVTITADKSTSTAYMEL | (VH) Anti- |
| | SSLRSEDTAVYYCARGGYYSHDMWSEDWGQGTLVTVSS | BCMA |
| 94 | LPVLTQPPSASGTPGQRVTISCSGRSSNIGSNSVNWYRQLPGA | Variable light |
| | APKLLIYSNNQRPPGVPVRFSGSKSGTSASLAISGLQSEDEAT | (VL) Anti- |
| | YYCATWDDNLNVHYVFGTGTKVTVLG | BCMA |
| 95 | QVQLVQSGSELKKPGASVKVSCKASGYTFTDYSINWVRQAP | Variable heavy |
| | GQGLEWMGWINTETREPAYAYDFRGRFVFSLDTSVSTAYLQ | (VH) Anti- |
| | ISSLKAEDTAVYYCARDYSYAMDYWGQGTLVTVSS | BCMA |
| 96 | DIVLTQSPASLAVSLGERATINCRASESVSVIGAHLIHWYQQK | Variable light |
| | PGQPPKLLIYLASNLETGVPARFSGSGSGTDFTLTISSLQAED | (VL) Anti- |
| | AAIYYCLQSRIFPRTFGQGTKLEIK | BCMA |
| 97 | EVQLVESGGGLVQPGGSLRLSCAVSGFALSNHGMSWVRRAP | Variable heavy |
| | GKGLEWVSGIVYSGSTYYAASVKGRFTISRDNSRNTLYLQM | (VH) Anti- |
| | NSLRPEDTAIYYCSAHGGESDVWGQGTTVTVSS | BCMA |
| 98 | DIQLTQSPSSLSASVGDRVTITCRASQSISSYLNWYQQKPGKA | Variable light |
| | PKLLIYAASSLQSGVPSRFSGSGSGTDFTLTISSLQPEDFATYY | (VL) Anti- |
| | CQQSYSTPYTFGQGTKVEIK | BCMA |
| 99 | QVQLVESGGGLVQPGRSLRLSCAASGFTFSNYAMSWVRQAP | Variable heavy |
| | GKGLGWVSGISRSGENTYYADSVKGRFTISRDNSKNTLYLQ | (VH) Anti- |
| | MNSLRDEDTAVYYCARSPAHYYGGMDVWGQGTTVTVSS | BCMA |
| 100 | DIVLTQSPGTLSLSPGERATLSCRASQSISSSFLAWYQQKPGQ | Variable light |
| | APRLLIYGASRRATGIPDRFSGSGSGTDFTLTISRLEPEDSAVY | (VL) Anti- |
| | YCQQYHSSPSWTFGQGTKLEIK | BCMA |
| 101 | QVQLVESGGGLVQPGGSLRLSCAVSGFALSNHGMSWVRRA | Variable heavy |
| | PGKGLEWVSGIVYSGSTYYAASVKGRFTISRDNSRNTLYLQ | (VH) Anti- |
| | MNSLRPEDTAIYYCSAHGGESDVWGQGTTVTVSS | BCMA |
| 102 | DIRLTQSPSPLSASVGDRVTITCQASEDINKFLNWYHQTPGKA | Variable light |
| | PKLLIYDASTLQTGVPSRFSGSGSGTDFTLTINSLQPEDIGTYY | (VL) Anti- |
| | CQQYESLPLTFGGGTKVEIK | BCMA |
| 103 | EVQLVESGGGLVQPGGSLRLSCAVSGFALSNHGMSWVRRAP | Variable heavy |
| | GKGLEWVSGIVYSGSTYYAASVKGRFTISRDNSRNTLYLQM | (VH) Anti- |
| | NSLRPEDTAIYYCSAHGGESDVWGQGTTVTVSS | BCMA |
| 104 | EIVLTQSPGTLSLSPGERATLSCRASQSIGSSSLAWYQQKPGQ | Variable light |
| | APRLLMYGASSRASGIPDRFSGSGSGTDFTLTISRLEPEDFAV | (VL) Anti- |
| | YYCQQYAGSPPFTFGQGTKVEIK | BCMA |
| 105 | QIQLVQSGPELKKPGETVKISCKASGYTFRHYSMNWVKQAP | Variable heavy |
| | GKGLKWMGRINTESGVPIYADDFKGRFAFSVETSASTAYLVI | (VH) Anti- |
| | NNLKDEDTASYFCSNDYLYSLDFWGQGTALTVSS | BCMA |
| 106 | DIVLTQSPPSLAMSLGKRATISCRASESVTILGSHLIYWYQQK | Variable light |
| | PGQPPTLLIQLASNVQTGVPARFSGSGSRTDFTLTIDPVEEDD | (VL) Anti- |
| | VAVYYCLQSRTIPRTFGGGTKLEIK | BCMA |
| 107 | QIQLVQSGPELKKPGETVKISCKASGYTFTHYSMNWVKQAP | Variable heavy |
| | GKGLKWMGRINTETGEPLYADDFKGRFAFSLETSASTAYLVI | (VH) Anti- |
| | NNLKNEDTATFFCSNDYLYSCDYWGQGTTLTVSS | BCMA |
| 108 | DIVLTQSPASLAMSLGKRATISCRASESVSVIGAHLIHWYQQ | Variable light |
| | KPGQPPKLLIYLASNLETGVPARFSGSGSGTDFTLTIDPVEED | (VL) Anti- |
| | DVAIYSCLQSRIFPRTFGGGTKLEIK | BCMA |
| 109 | QVQLVQSGAEVKKPGASVKVSCKASGYSFPDYYINWVRQA | Variable heavy |
| | PGQGLEWMGWIYFASGNSEYNQKFTGRVTMTRDTSINTAY | (VH) Anti- |
| | MELSSLTSEDTAVYFCASLYDYDWYFDVWGQGTMVTVSS | BCMA |
| 110 | DIVMTQTPLSLSVTPGQPASISCKSSQSLVHSNGNTYLHWYL | Variable light |
| | QKPGQSPQLLIYKVSNRFSGVPDRFSGSGSGTDFTLKISRVEA | (VL) Anti- |
| | EDVGIYYCSQSSIYPWTFGQGTKLEIK | BCMA |
| 111 | QVQLVQSGAEVKKPGASVKVSCKASGYSFPDYYINWVRQA | Variable heavy |
| | PGQGLEWMGWIYFASGNSEYNQKFTGRVTMTRDTSSSTAY | (VH) Anti- |
| | MELSSLRSEDTAVYFCASLYDYDWYFDVWGQGTMVTVSS | BCMA |
| 112 | DIVMTQTPLSLSVTPGEPASISCKSSQSLVHSNGNTYLHWYL | Variable light |
| | QKPGQSPQLLIYKVSNRFSGVPDRFSGSGSGADFTLKISRVEA | (VL) Anti- |
| | EDVGVYYCAETSHVPWTFGQGTKLEIK | BCMA |
| 113 | QVQLVESGGGLVQPGGSLRLSCEASGFTLDYYAIGWFRQAP | Anti-BCMA |
| | GKEREGVICISRSDGSTYYADSVKGRFTISRDNAKKTVYLQM | sdAb |
| | ISLKPEDTAAYYCAAGADCSGYLRDYEFRGQGTQVTVSS | |
| 114 | IEVMYPPPYLDNEKSNGTIIHVKGKHLCPSPLFPGPSKP | CD28 spacer |
| 115 | IYIWAPLAGTCGVLLLSLVITLYCN | CD8a TM |
| 116 | LDNEKSNGTIIHVKGKHLCPSPLFPGPSKP | CD28 spacer |
| | | (truncated) |
| 117 | PTTTPAPRPPTPAPTIASQPLSLRPEACRPAAGGAVHTRGLDF | CD8a hinge |
| | ACD | |
| 118 | TTTPAPRPPTPAPTIASQPLSLRPEACRPAAGGAVHTRGLDFA | CD8a hinge |
| | CD | |
| 119 | FVPVFLPAKPTTTPAPRPPTPAPTIASQPLSLRPEACRPAAGGA | CD8a hinge |
| | VHTRGLDFACD | |
| 120 | DTGLYICKVELMYPPPYYLGIGNGTQIYVIDPEPCPDSD | CTLA4 hinge |
| 121 | FLLWILAAVSSGLFFYSFLLTAVS | CTLA4 TM |
| 122 | QIKESLRAELRVTERRAEVPTAHPSPSPRPAGQFQTLV | PD-1 hinge |
| 123 | VGVVGGLLGSLVLLVWVLAVI | PD-1 TM |
| 124 | GLAVSTISSFFPPGYQ | FcγRIIIa hinge |
| 125 | EPKSPDKTHTCPPCPAPPVAGPSVFLFPPKPKDTLMIARTPEV | IgG1 hinge |
| | TCVVVDVSHEDPEVKFNWYVDGVEVHNAKTKPREEQYNST | |
| | YRVVSVLTVLHQDWLNGKEYKCKVSNKALPAPIEKTISKAK | |
| | GQPREPQVYTLPPSRDELTKNQVSLTCLVKGFYPSDIAVEWE | |
| | SNGQPENNYKTTPPVLDSDGSFFLYSKLTVDKSRWQQGNVF | |
| | SCSVMHEALHNHYTQKSLSLSPGK | |
| 126 | EVQLLESGGGLVQPGGSLRLSCAASGFTFSSYAMSWVRQAP | anti-BCMA CAR |
| | GKGLEWVSAISGSGGSTYYADSVKGRFTISRDNSKNTLYLQ | |
| | MNSLRAEDTAVYYCARAEMGAVFDIWGQGTMVTVSSGSTS | |
| | GSGKPGSGEGSTKGEIVLTQSPATLSLSPGERATLSCRASQSV | |
| | SRYLAWYQQKPGQAPRLLIYDASNRATGIPARFSGSGSGTDF | |
| | TLTISSLEPEDFAVYYCQQRISWPFTFGGGTKVEIKRAAALDN | |
| | EKSNGTIIHVKGKHLCPSPLFPGPSKPFWVLVVVGGVLACYS | |
| | LLVTVAFIIFWVRSKRSRLLHSDYMNMTPRRPGPTRKHYQPY | |
| | APPRDFAAYRSRVKFSRSADAPAYQQGQNQLYNELNLGRRE | |
| | EYDVLDKRRGRDPEMGGKPRRKNPQEGLYNELQKDKMAEA | |
| | YSEIGMKGERRRGKGHDGLYQGLSTATKDTYDALHMQALP | |
| | PR | |
| 127 | EIVLTQSPATLSLSPGERATLSCRASQSVSRYLAWYQQKPGQ | anti-BCMA CAR |
| | APRLLIYDASNRATGIPARFSGSGSGTDFTLTISSLEPEDFAVY | |
| | YCQQRISWPFTFGGGTKVEIKRGSTSGSGKPGSGEGSTKGEV | |
| | QLLESGGGLVQPGGSLRLSCAASGFTFSSYAMSWVRQAPGK | |
| | GLEWVSAISGSGGSTYYADSVKGRFTISRDNSKNTLYLQMNS | |
| | LRAEDTAVYYCARAEMGAVFDIWGQGTMVTVSSAAALDNE | |
| | KSNGTIIHVKGKHLCPSPLFPGPSKPFWVLVVVGGVLACYSL | |
| | LVTVAFIIFWVRSKRSRLLHSDYMNMTPRRPGPTRKHYQPY | |
| | APPRDFAAYRSRVKFSRSADAPAYQQGQNQLYNELNLGRRE | |
| | EYDVLDKRRGRDPEMGGKPRRKNPQEGLYNELQKDKMAEA | |
| | YSEIGMKGERRRGKGHDGLYQGLSTATKDTYDALHMQALP | |
| | PR | |
| 128 | QVQLVESGGGVVQPGRSLRLSCAASGFTFSSYGMHWVRQAP | anti-BCMA CAR |
| | GKGLEWVAVISYDGSNKYYADSVKGRFTISRDNSKNTLYLQ | |
| | MNSLRAEDTAVYYCARDGTYLGGLWYFDLWGRGTLVTVSS | |
| | GSTSGSGKPGSGEGSTKGDIVMTQSPLSLPVTPGEPASISCRSS | |
| | QSLLHSNGYNYLDWYLQKPGQSPQLLIYLGSNRASGVPDRF | |
| | SGSGSGTDFTLKISRVEAEDVGVYYCMQGLGLPLTFGGGTK | |
| | VEIKRAAALDNEKSNGTIIHVKGKHLCPSPLFPGPSKPFWVLV | |
| | VVGGVLACYSLLVTVAFIIFWVRSKRSRLLHSDYMNMTPRR | |
| | PGPTRKHYQPYAPPRDFAAYRSRVKFSRSADAPAYQQGQNQ | |
| | LYNELNLGRREEYDVLDKRRGRDPEMGGKPRRKNPQEGLY | |
| | NELQKDKMAEAYSEIGMKGERRRGKGHDGLYQGLSTATKD | |
| | TYDALHMQALPPR | |
| 129 | DIVMTQSPLSLPVTPGEPASISCRSSQSLLHSNGYNYLDWYLQ | anti-BCMA CAR |
| | KPGQSPQLLIYLGSNRASGVPDRFSGSGSGTDFTLKISRVEAE | |
| | DVGVYYCMQGLGLPLTFGGGTKVEIKRGSTSGSGKPGSGEG | |
| | STKGQVQLVESGGGVVQPGRSLRLSCAASGFTFSSYGMHWV | |
| | RQAPGKGLEWVAVISYDGSNKYYADSVKGRFTISRDNSKNT | |
| | LYLQMNSLRAEDTAVYYCARDGTYLGGLWYFDLWGRGTL | |
| | VTVSSAAALDNEKSNGTIIHVKGKHLCPSPLFPGPSKPFWVL | |
| | VVVGGVLACYSLLVTVAFIIFWVRSKRSRLLHSDYMNMTPR | |
| | RPGPTRKHYQPYAPPRDFAAYRSRVKFSRSADAPAYQQGQN | |
| | QLYNELNLGRREEYDVLDKRRGRDPEMGGKPRRKNPQEGL | |
| | YNELQKDKMAEAYSEIGMKGERRRGKGHDGLYQGLSTATK | |
| | DTYDALHMQALPPR | |
| 130 | QVQLVQSGAEVKKPGASVKVSCKASGYTFTSYYMHWVRQA | anti-BCMA CAR |
| | PGQGLEWMGIINPGGGSTSYAQKFQGRVTMTRDTSTSTVYM | |
| | ELSSLRSEDTAVYYCARESWPMDVWGQGTTVTVSSGSTSGS | |
| | GKPGSGEGSTKGEIVMTQSPATLSVSPGERATLSCRASQSVSS | |
| | NLAWYQQKPGQAPRLLIYGASTRATGIPARFSGSGSGTEFTL | |
| | TISSLQSEDFAVYYCQQYAAYPTFGGGTKVEIKRAAALDNEK | |
| | SNGTIIHVKGKHLCPSPLFPGPSKPFWVLVVVGGVLACYSLL | |
| | VTVAFIIFWVRSKRSRLLHSDYMNMTPRRPGPTRKHYQPYAP | |
| | PRDFAAYRSRVKFSRSADAPAYQQGQNQLYNELNLGRREEY | |
| | DVLDKRRGRDPEMGGKPRRKNPQEGLYNELQKDKMAEAYS | |
| | EIGMKGERRRGKGHDGLYQGLSTATKDTYDALHMQALPPR | |
| 131 | EIVMTQSPATLSVSPGERATLSCRASQSVSSNLAWYQQKPGQ | anti-BCMA CAR |
| | APRLLIYGASTRATGIPARFSGSGSGTEFTLTISSLQSEDFAVY | |
| | YCQQYAAYPTFGGGTKVEIKRGSTSGSGKPGSGEGSTKGQV | |
| | QLVQSGAEVKKPGASVKVSCKASGYTFTSYYMHWVRQAPG | |
| | QGLEWMGIINPGGGSTSYAQKFQGRVTMTRDTSTSTVYMEL | |
| | SSLRSEDTAVYYCARESWPMDVWGQGTTVTVSSAAALDNE | |
| | KSNGTIIHVKGKHLCPSPLFPGPSKPFWVLVVVGGVLACYSL | |
| | LVTVAFIIFWVRSKRSRLLHSDYMNMTPRRPGPTRKHYQPY | |
| | APPRDFAAYRSRVKFSRSADAPAYQQGQNQLYNELNLGRRE | |
| | EYDVLDKRRGRDPEMGGKPRRKNPQEGLYNELQKDKMAEA | |
| | YSEIGMKGERRRGKGHDGLYQGLSTATKDTYDALHMQALP | |
| | PR | |
| 132 | QLQLQESGPGLVKPSETLSLTCTVSGGSISSSSYYWGWIRQPP | anti-BCMA CAR |
| | GKGLEWIGSISYSGSTYYNPSLKSRVTISVDTSKNQFSLKLSS | |
| | VTAADTAVYYCARGRGYATSLAFDIWGQGTMVTVSSGSTS | |
| | GSGKPGSGEGSTKGEIVLTQSPATLSLSPGERATLSCRASQSV | |
| | SSYLAWYQQKPGQAPRLLIYDASNRATGIPARFSGSGSGTDF | |
| | TLTISSLEPEDFAVYYCQQRHVWPPTFGGGTKVEIKRAAALD | |
| | NEKSNGTIIHVKGKHLCPSPLFPGPSKPFWVLVVVGGVLACY | |
| | SLLVTVAFIIFWVRSKRSRLLHSDYMNMTPRRPGPTRKHYQP | |
| | YAPPRDFAAYRSRVKFSRSADAPAYQQGQNQLYNELNLGRR | |
| | EEYDVLDKRRGRDPEMGGKPRRKNPQEGLYNELQKDKMAE | |
| | AYSEIGMKGERRRGKGHDGLYQGLSTATKDTYDALHMQAL | |
| | PPR | |
| 133 | EIVLTQSPATLSLSPGERATLSCRASQSVSSYLAWYQQKPGQ | anti-BCMA CAR |
| | APRLLIYDASNRATGIPARFSGSGSGTDFTLTISSLEPEDFAVY | |
| | YCQQRHVWPPTFGGGTKVEIKRGSTSGSGKPGSGEGSTKGQ | |
| | LQLQESGPGLVKPSETLSLTCTVSGGSISSSSYYWGWIRQPPG | |
| | KGLEWIGSISYSGSTYYNPSLKSRVTISVDTSKNQFSLKLSSV | |
| | TAADTAVYYCARGRGYATSLAFDIWGQGTMVTVSSAAALD | |
| | NEKSNGTIIHVKGKHLCPSPLFPGPSKPFWVLVVVGGVLACY | |
| | SLLVTVAFIIFWVRSKRSRLLHSDYMNMTPRRPGPTRKHYQP | |
| | YAPPRDFAAYRSRVKFSRSADAPAYQQGQNQLYNELNLGRR | |
| | EEYDVLDKRRGRDPEMGGKPRRKNPQEGLYNELQKDKMAE | |
| | AYSEIGMKGERRRGKGHDGLYQGLSTATKDTYDALHMQAL | |
| | PPR | |
| 134 | EVQLVESGGGLVQPGGSLRLSCAASGFTFSSYSMNWVRQAP | anti-BCMA CAR |
| | GKGLEWVSTISSSSSTIYYADSVKGRFTISRDNAKNSLYLQM | |
| | NSLRAEDTAVYYCARGSQEHLIFDYWGQGTLVTVSSGSTSG | |
| | SGKPGSGEGSTKGEIVLTQSPATLSLSPGERATLSCRASQSVS | |
| | RYLAWYQQKPGQAPRLLIYDASNRATGIPARFSGSGSGTDFT | |
| | LTISSLEPEDFAVYYCQQRFYYPWTFGGGTKVEIKRAAALDN | |
| | EKSNGTIIHVKGKHLCPSPLFPGPSKPFWVLVVVGGVLACYS | |
| | LLVTVAFIIFWVRSKRSRLLHSDYMNMTPRRPGPTRKHYQPY | |
| | APPRDFAAYRSRVKFSRSADAPAYQQGQNQLYNELNLGRRE | |
| | EYDVLDKRRGRDPEMGGKPRRKNPQEGLYNELQKDKMAEA | |
| | YSEIGMKGERRRGKGHDGLYQGLSTATKDTYDALHMQALP | |
| | PR | |
| 135 | EIVLTQSPATLSLSPGERATLSCRASQSVSRYLAWYQQKPGQ | anti-BCMA CAR |
| | APRLLIYDASNRATGIPARFSGSGSGTDFTLTISSLEPEDFAVY | |
| | YCQQRFYYPWTFGGGTKVEIKRGSTSGSGKPGSGEGSTKGE | |
| | VQLVESGGGLVQPGGSLRLSCAASGFTFSSYSMNWVRQAPG | |
| | KGLEWVSTISSSSSTIYYADSVKGRFTISRDNAKNSLYLQMNS | |
| | LRAEDTAVYYCARGSQEHLIFDYWGQGTLVTVSSAAALDNE | |
| | KSNGTIIHVKGKHLCPSPLFPGPSKPFWVLVVVGGVLACYSL | |
| | LVTVAFIIFWVRSKRSRLLHSDYMNMTPRRPGPTRKHYQPY | |
| | APPRDFAAYRSRVKFSRSADAPAYQQGQNQLYNELNLGRRE | |
| | EYDVLDKRRGRDPEMGGKPRRKNPQEGLYNELQKDKMAEA | |
| | YSEIGMKGERRRGKGHDGLYQGLSTATKDTYDALHMQALP | |
| | PR | |
| 136 | QVQLVESGGGVVQPGRSLRLSCAASGFTFSSYGMHWVRQAP | anti-BCMA CAR |
| | GKGLEWVAVISYDGSNKYYADSVKGRFTISRDNSKNTLYLQ | |
| | MNSLRAEDTAVYYCARTDFWSGSPPGLDYWGQGTLVTVSS | |
| | GSTSGSGKPGSGEGSTKGDIQLTQSPSSVSASVGDRVTITCRA | |
| | SQGISSWLAWYQQKPGKAPKLLIYGASSLQSGVPSRFSGSGS | |
| | GTDFTLTISSLQPEDFATYYCQQIYTFPFTFGGGTKVEIKRAA | |
| | ALDNEKSNGTIIHVKGKHLCPSPLFPGPSKPFWVLVVVGGVL | |
| | ACYSLLVTVAFIIFWVRSKRSRLLHSDYMNMTPRRPGPTRKH | |
| | YQPYAPPRDFAAYRSRVKFSRSADAPAYQQGQNQLYNELNL | |
| | GRREEYDVLDKRRGRDPEMGGKPRRKNPQEGLYNELQKDK | |
| | MAEAYSEIGMKGERRRGKGHDGLYQGLSTATKDTYDALHM | |
| | QALPPR | |
| 137 | DIQLTQSPSSVSASVGDRVTITCRASQGISSWLAWYQQKPGK | anti-BCMA CAR |
| | APKLLIYGASSLQSGVPSRFSGSGSGTDFTLTISSLQPEDFATY | |
| | YCQQIYTFPFTFGGGTKVEIKRGSTSGSGKPGSGEGSTKGQV | |
| | QLVESGGGVVQPGRSLRLSCAASGFTFSSYGMHWVRQAPGK | |
| | GLEWVAVISYDGSNKYYADSVKGRFTISRDNSKNTLYLQMN | |
| | SLRAEDTAVYYCARTDFWSGSPPGLDYWGQGTLVTVSSAA | |
| | ALDNEKSNGTIIHVKGKHLCPSPLFPGPSKPFWVLVVVGGVL | |
| | ACYSLLVTVAFIIFWVRSKRSRLLHSDYMNMTPRRPGPTRKH | |
| | YQPYAPPRDFAAYRSRVKFSRSADAPAYQQGQNQLYNELNL | |
| | GRREEYDVLDKRRGRDPEMGGKPRRKNPQEGLYNELQKDK | |
| | MAEAYSEIGMKGERRRGKGHDGLYQGLSTATKDTYDALHM | |
| | QALPPR | |
| 138 | QVQLVQSGAEVKKPGSSVKVSCKASGGTFSSYAISWVRQAP | anti-BCMA CAR |
| | GQGLEWMGGIIPIFGTANYAQKFQGRVTITADESTSTAYMEL | |
| | SSLRSEDTAVYYCARTPEYSSSIWHYYYGMDVWGQGTTVT | |
| | VSSGSTSGSGKPGSGEGSTKGDIVMTQSPDSLAVSLGERATIN | |
| | CKSSQSVLYSSNNKNYLAWYQQKPGQPPKLLIYWASTRESG | |
| | VPDRFSGSGSGTDFTLTISSLQAEDVAVYYCQQFAHTPFTFG | |
| | GGTKVEIKRAAALDNEKSNGTIIHVKGKHLCPSPLFPGPSKPF | |
| | WVLVVVGGVLACYSLLVTVAFIIFWVRSKRSRLLHSDYMN | |
| | MTPRRPGPTRKHYQPYAPPRDFAAYRSRVKFSRSADAPAYQ | |
| | QGQNQLYNELNLGRREEYDVLDKRRGRDPEMGGKPRRKNP | |
| | QEGLYNELQKDKMAEAYSEIGMKGERRRGKGHDGLYQGLS | |
| | TATKDTYDALHMQALPPR | |
| 139 | DIVMTQSPDSLAVSLGERATINCKSSQSVLYSSNNKNYLAWY | anti-BCMA CAR |
| | QQKPGQPPKLLIYWASTRESGVPDRFSGSGSGTDFTLTISSLQ | |
| | AEDVAVYYCQQFAHTPFTFGGGTKVEIKRGSTSGSGKPGSGE | |
| | GSTKGQVQLVQSGAEVKKPGSSVKVSCKASGGTFSSYAISW | |
| | VRQAPGQGLEWMGGIIPIFGTANYAQKFQGRVTITADESTST | |
| | AYMELSSLRSEDTAVYYCARTPEYSSSIWHYYYGMDVWGQ | |
| | GTTVTVSSAAALDNEKSNGTIIHVKGKHLCPSPLFPGPSKPFW | |
| | VLVVVGGVLACYSLLVTVAFIIFWVRSKRSRLLHSDYMNMT | |
| | PRRPGPTRKHYQPYAPPRDFAAYRSRVKFSRSADAPAYQQG | |
| | QNQLYNELNLGRREEYDVLDKRRGRDPEMGGKPRRKNPQE | |
| | GLYNELQKDKMAEAYSEIGMKGERRRGKGHDGLYQGLSTA | |
| | TKDTYDALHMQALPPR | |
| 140 | QVQLVESGGGVVQPGRSLRLSCAASGFTFSSYGMHWVRQAP | anti-BCMA CAR |
| | GKGLEWVAVISYDGSNKYYADSVKGRFTISRDNSKNTLYLQ | |
| | MNSLRAEDTAVYYCVKGPLQEPPYDYGMDVWGQGTTVTV | |
| | SSGSTSGSGKPGSGEGSTKGEIVMTQSPATLSVSPGERATLSC | |
| | RASQSVSSNLAWYQQKPGQAPRLLIYSASTRATGIPARFSGS | |
| | GSGTEFTLTISSLQSEDFAVYYCQQHHVWPLTFGGGTKVEIK | |
| | RAAALDNEKSNGTIIHVKGKHLCPSPLFPGPSKPFWVLVVVG | |
| | GVLACYSLLVTVAFIIFWVRSKRSRLLHSDYMNMTPRRPGPT | |
| | RKHYQPYAPPRDFAAYRSRVKFSRSADAPAYQQGQNQLYN | |
| | ELNLGRREEYDVLDKRRGRDPEMGGKPRRKNPQEGLYNEL | |
| | QKDKMAEAYSEIGMKGERRRGKGHDGLYQGLSTATKDTYD | |
| | ALHMQALPPR | |
| 141 | EIVMTQSPATLSVSPGERATLSCRASQSVSSNLAWYQQKPGQ | anti-BCMA CAR |
| | APRLLIYSASTRATGIPARFSGSGSGTEFTLTISSLQSEDFAVY | |
| | YCQQHHVWPLTFGGGTKVEIKRGSTSGSGKPGSGEGSTKGQ | |
| | VQLVESGGGVVQPGRSLRLSCAASGFTFSSYGMHWVRQAPG | |
| | KGLEWVAVISYDGSNKYYADSVKGRFTISRDNSKNTLYLQM | |
| | NSLRAEDTAVYYCVKGPLQEPPYDYGMDVWGQGTTVTVSS | |
| | AAALDNEKSNGTIIHVKGKHLCPSPLFPGPSKPFWVLVVVGG | |
| | VLACYSLLVTVAFIIFWVRSKRSRLLHSDYMNMTPRRPGPTR | |
| | KHYQPYAPPRDFAAYRSRVKFSRSADAPAYQQGQNQLYNEL | |
| | NLGRREEYDVLDKRRGRDPEMGGKPRRKNPQEGLYNELQK | |
| | DKMAEAYSEIGMKGERRRGKGHDGLYQGLSTATKDTYDAL | |
| | HMQALPPR | |
| 142 | QSALTQPASVSASPGQSIAISCTGTSSDVGWYQQHPGKAPKL | anti-BCMA CAR |
| | MIYEDSKRPSGVSNRFSGSKSGNTASLTISGLQAEDEADYYC | |
| | SSNTRSSTLVFGGGTKLTVLGSRGGGGSGGGGSGGGGSLEM | |
| | AEVQLVQSGAEMKKPGASLKLSCKASGYTFIDYYVYWMRQ | |
| | APGQGLESMGWINPNSGGTNYAQKFQGRVTMTRDTSISTAY | |
| | MELSRLRSDDTAMYYCARSQRDGYMDYWGQGTLVTVSSA | |
| | AAIEVMYPPPYLDNEKSNGTIIHVKGKHLCPSPLFPGPSKPFW | |
| | VLVVVGGVLACYSLLVTVAFIIFWVRSKRSRLLHSDYMNMT | |
| | PRRPGPTRKHYQPYAPPRDFAAYRSRVKFSRSADAPAYQQG | |
| | QNQLYNELNLGRREEYDVLDKRRGRDPEMGGKPRRKNPQE | |
| | GLYNELQKDKMAEAYSEIGMKGERRRGKGHDGLYQGLSTA | |
| | TKDTYDALHMQALPPR | |
| 143 | QSVLTQPPSVSGAPGQRVTISCTGSSSNIGAGFDVHWYQQLP | anti-BCMA CAR |
| | GTAPKLLIYGNSNRPSGVPDRFSGSKSGTSASLAITGLQAEDE | |
| | ADYYCQSYDSSLSGYVFGTGTKVTVLGSRGGGGSGGGGSG | |
| | GGGSLEMAQVQLVQSGAEVKKPGASVKVSCKASGYTFTDY | |
| | YMHWVRQAPGQRLEWMGWINPNSGGTNYAQKFQDRITVT | |
| | RDTSSNTGYMELTRLRSDDTAVYYCARSPYSGVLDKWGQG | |
| | TLVTVSSAAAIEVMYPPPYLDNEKSNGTIIHVKGKHLCPSPLF | |
| | PGPSKPFWVLVVVGGVLACYSLLVTVAFIIFWVRSKRSRLLH | |
| | SDYMNMTPRRPGPTRKHYQPYAPPRDFAAYRSRVKFSRSAD | |
| | APAYQQGQNQLYNELNLGRREEYDVLDKRRGRDPEMGGKP | |
| | RRKNPQEGLYNELQKDKMAEAYSEIGMKGERRRGKGHDGL | |
| | YQGLSTATKDTYDALHMQALPPR | |
| 144 | SYELTQPPSASGTPGQRVTMSCSGTSSNIGSHSVNWYQQLPG | anti-BCMA CAR |
| | TAPKLLIYTNNQRPSGVPDRFSGSKSGTSASLAISGLQSEDEA | |
| | DYYCAAWDGSLNGLVFGGGTKLTVLGSRGGGGSGGGGSGG | |
| | GGSLEMAEVQLVQSGAEVKKPGESLKISCKGSGYSFTSYWIG | |
| | WVRQMPGKGLEWMGIIYPGDSDTRYSPSFQGHVTISADKSIS | |
| | TAYLQWSSLKASDTAMYYCARYSGSFDNWGQGTLVTVSSA | |
| | AAIEVMYPPPYLDNEKSNGTIIHVKGKHLCPSPLFPGPSKPFW | |
| | VLVVVGGVLACYSLLVTVAFIIFWVRSKRSRLLHSDYMNMT | |
| | PRRPGPTRKHYQPYAPPRDFAAYRSRVKFSRSADAPAYQQG | |
| | QNQLYNELNLGRREEYDVLDKRRGRDPEMGGKPRRKNPQE | |
| | GLYNELQKDKMAEAYSEIGMKGERRRGKGHDGLYQGLSTA | |
| | TKDTYDALHMQALPPR | |
| 145 | LPVLTQPPSASGTPGQRVTISCSGRSSNIGSNSVNWYRQLPGA | anti-BCMA CAR |
| | APKLLIYSNNQRPPGVPVRFSGSKSGTSASLAISGLQSEDEAT | |
| | YYCATWDDNLNVHYVFGTGTKVTVLGSRGGGGSGGGGSG | |
| | GGGSLEMAQVQLVQSGAEVKKPGSSVKVSCKASGGTFSSYA | |
| | ISWVRQAPGQGLEWMGRIIPILGIANYAQKFQGRVTITADKS | |
| | TSTAYMELSSLRSEDTAVYYCARGGYYSHDMWSEDWGQGT | |
| | LVTVSSAAAIEVMYPPPYLDNEKSNGTIIHVKGKHLCPSPLFP | |
| | GPSKPFWVLVVVGGVLACYSLLVTVAFIIFWVRSKRSRLLHS | |
| | DYMNMTPRRPGPTRKHYQPYAPPRDFAAYRSRVKFSRSADA | |
| | PAYQQGQNQLYNELNLGRREEYDVLDKRRGRDPEMGGKPR | |
| | RKNPQEGLYNELQKDKMAEAYSEIGMKGERRRGKGHDGLY | |
| | QGLSTATKDTYDALHMQALPPR | |
| 146 | QAVLTQPPSASGTPGQRVTISCSGSSSNIGSNYVFWYQQLPGT | anti-BCMA CAR |
| | APKLLIYSNNQRPSGVPDRFSGSKSGTSASLAISGLRSEDEAD | |
| | YYCAAWDDSLSASYVFGTGTKVTVLGSRGGGGSGGGGSGG | |
| | GGSLEMAQVQLVQSGAEVKKPGSSVKVSCKASGGTFSSYAI | |
| | SWVRQAPGQGLEWMGRIIPILGTANYAQKFQGRVTITADEST | |
| | STAYMELSSLRSEDTAVYYCARSGYGSYRWEDSWGQGTLV | |
| | TVSSAAAIEVMYPPPYLDNEKSNGTIIHVKGKHLCPSPLFPGP | |
| | SKPFWVLVVVGGVLACYSLLVTVAFIIFWVRSKRSRLLHSDY | |
| | MNMTPRRPGPTRKHYQPYAPPRDFAAYRSRVKFSRSADAPA | |
| | YQQGQNQLYNELNLGRREEYDVLDKRRGRDPEMGGKPRRK | |
| | NPQEGLYNELQKDKMAEAYSEIGMKGERRRGKGHDGLYQG | |
| | LSTATKDTYDALHMQALPPR | |
| 147 | LPVLTQPPSASGTPGQRVTISCSGRSSNIGSNSVNWYRQLPGA | anti-BCMA CAR |
| | APKLLIYSNNQRPPGVPVRFSGSKSGTSASLAISGLQSEDEAT | |
| | YYCATWDDNLNVHYVFGTGTKVTVLGSRGGGGSGGGGSG | |
| | GGGSLEMAQVQLVQSGAEVKKPGSSVKVSCKASGGTFSSYA | |
| | ISWVRQAPGQGLEWMGRIIPILGIANYAQKFQGRVTITADKS | |
| | TSTAYMELSSLRSEDTAVYYCARGGYYSHDMWSEDWGQGT | |
| | LVTVSSAAAPTTTPAPRPPTPAPTIASQPLSLRPEACRPAAGG | |
| | AVHTRGLDFACDIYIWAPLAGTCGVLLLSLVITLYCNKRGRK | |
| | KLLYIFKQPFMRPVQTTQEEDGCSCRFPEEEEGGCELRVKFS | |
| | RSAEPPAYQQGQNQLYNELNLGRREEYDVLDKRRGRDPEM | |
| | GGKPRRKNPQEGLYNELQKDKMAEAYSEIGMKGERRRGKG | |
| | HDGLYQGLSTATKDTYDALHMQALPPR | |
| 148 | SYELTQPPSASGTPGQRVTMSCSGTSSNIGSHSVNWYQQLPG | anti-BCMA CAR |
| | TAPKLLIYTNNQRPSGVPDRFSGSKSGTSASLAISGLQSEDEA | |
| | DYYCAAWDGSLNGLVFGGGTKLTVLGSRGGGGSGGGGSGG | |
| | GGSLEMAEVQLVQSGAEVKKPGESLKISCKGSGYSFTSYWIG | |
| | WVRQMPGKGLEWMGIIYPGDSDTRYSPSFQGHVTISADKSIS | |
| | TAYLQWSSLKASDTAMYYCARYSGSFDNWGQGTLVTVSSA | |
| | AAPTTTPAPRPPTPAPTIASQPLSLRPEACRPAAGGAVHTRGL | |
| | DFACDIYIWAPLAGTCGVLLLSLVITLYCNKRGRKKLLYIFK | |
| | QPFMRPVQTTQEEDGCSCRFPEEEEGGCELRVKFSRSAEPPA | |
| | YQQGQNQLYNELNLGRREEYDVLDKRRGRDPEMGGKPRRK | |
| | NPQEGLYNELQKDKMAEAYSEIGMKGERRRGKGHDGLYQG | |
| | LSTATKDTYDALHMQALPPR | |
| 149 | QAVLTQPPSASGTPGQRVTISCSGSSSNIGSNYVFWYQQLPGT | anti-BCMA CAR |
| | APKLLIYSNNQRPSGVPDRFSGSKSGTSASLAISGLRSEDEAD | |
| | YYCAAWDDSLSASYVFGTGTKVTVLGSRGGGGSGGGGSGG | |
| | GGSLEMAQVQLVQSGAEVKKPGSSVKVSCKASGGTFSSYAI | |
| | SWVRQAPGQGLEWMGRIIPILGTANYAQKFQGRVTITADEST | |
| | STAYMELSSLRSEDTAVYYCARSGYGSYRWEDSWGQGTLV | |
| | TVSSAAAPTTTPAPRPPTPAPTIASQPLSLRPEACRPAAGGAV | |
| | HTRGLDFACDIYIWAPLAGTCGVLLLSLVITLYCNKRGRKKL | |
| | LYIFKQPFMRPVQTTQEEDGCSCRFPEEEEGGCELRVKFSRSA | |
| | EPPAYQQGQNQLYNELNLGRREEYDVLDKRRGRDPEMGGK | |
| | PRRKNPQEGLYNELQKDKMAEAYSEIGMKGERRRGKGHDG | |
| | LYQGLSTATKDTYDALHMQALPPR | |
| 150 | QSVLTQPPSVSGAPGQRVTISCTGSSSNIGAGFDVHWYQQLP | anti-BCMA CAR |
| | GTAPKLLIYGNSNRPSGVPDRFSGSKSGTSASLAITGLQAEDE | |
| | ADYYCQSYDSSLSGYVFGTGTKVTVLGSRGGGGSGGGGSG | |
| | GGGSLEMAQVQLVQSGAEVKKPGASVKVSCKASGYTFTDY | |
| | YMHWVRQAPGQRLEWMGWINPNSGGTNYAQKFQDRITVT | |
| | RDTSSNTGYMELTRLRSDDTAVYYCARSPYSGVLDKWGQG | |
| | TLVTVSSAAAPTTTPAPRPPTPAPTIASQPLSLRPEACRPAAG | |
| | GAVHTRGLDFACDIYIWAPLAGTCGVLLLSLVITLYCNKRGR | |
| | KKLLYIFKQPFMRPVQTTQEEDGCSCRFPEEEEGGCELRVKF | |
| | SRSAEPPAYQQGQNQLYNELNLGRREEYDVLDKRRGRDPE | |
| | MGGKPRRKNPQEGLYNELQKDKMAEAYSEIGMKGERRRGK | |
| | GHDGLYQGLSTATKDTYDALHMQALPPR | |
| 151 | QSALTQPASVSASPGQSIAISCTGTSSDVGWYQQHPGKAPKL | anti-BCMA CAR |
| | MIYEDSKRPSGVSNRFSGSKSGNTASLTISGLQAEDEADYYC | |
| | SSNTRSSTLVFGGGTKLTVLGSRGGGGSGGGGSGGGGSLEM | |
| | AEVQLVQSGAEMKKPGASLKLSCKASGYTFIDYYVYWMRQ | |
| | APGQGLESMGWINPNSGGTNYAQKFQGRVTMTRDTSISTAY | |
| | MELSRLRSDDTAMYYCARSQRDGYMDYWGQGTLVTVSSA | |
| | AAPTTTPAPRPPTPAPTIASQPLSLRPEACRPAAGGAVHTRGL | |
| | DFACDIYIWAPLAGTCGVLLLSLVITLYCNKRGRKKLLYIFK | |
| | QPFMRPVQTTQEEDGCSCRFPEEEEGGCELRVKFSRSAEPPA | |
| | YQQGQNQLYNELNLGRREEYDVLDKRRGRDPEMGGKPRRK | |
| | NPQEGLYNELQKDKMAEAYSEIGMKGERRRGKGHDGLYQG | |
| | LSTATKDTYDALHMQALPPR | |
| 152 | DIVLTQSPPSLAMSLGKRATISCRASESVTILGSHLIHWYQQK | anti-BCMA CAR |
| | PGQPPTLLIQLASNVQTGVPARFSGSGSRTDFTLTIDPVEEDD | |
| | VAVYYCLQSRTIPRTFGGGTKLEIKGSTSGSGKPGSGEGSTK | |
| | GQIQLVQSGPELKKPGETVKISCKASGYTFTDYSINWVKRAP | |
| | GKGLKWMGWINTETREPAYAYDFRGRFAFSLETSASTAYLQ | |
| | INNLKYEDTATYFCALDYSYAMDYWGQGTSVTVSSAAATT | |
| | TPAPRPPTPAPTIASQPLSLRPEACRPAAGGAVHTRGLDFACD | |
| | IYIWAPLAGTCGVLLLSLVITLYCKRGRKKLLYIFKQPFMRPV | |
| | QTTQEEDGCSCRFPEEEEGGCELRVKFSRSADAPAYQQGQN | |
| | QLYNELNLGRREEYDVLDKRRGRDPEMGGKPRRKNPQEGL | |
| | YNELQKDKMAEAYSEIGMKGERRRGKGHDGLYQGLSTATK | |
| | DTYDALHMQALPPR | |
| 153 | DIVLTQSPASLAVSLGERATINCRASESVSVIGAHLIHWYQQK | anti-BCMA CAR |
| | PGQPPKLLIYLASNLETGVPARFSGSGSGTDFTLTISSLQAED | |
| | AAIYYCLQSRIFPRTFGQGTKLEIKGSTSGSGKPGSGEGSTKG | |
| | QVQLVQSGSELKKPGASVKVSCKASGYTFTDYSINWVRQAP | |
| | GQGLEWMGWINTETREPAYAYDFRGRFVFSLDTSVSTAYLQ | |
| | ISSLKAEDTAVYYCARDYSYAMDYWGQGTLVTVSSAAATT | |
| | TPAPRPPTPAPTIASQPLSLRPEACRPAAGGAVHTRGLDFACD | |
| | IYIWAPLAGTCGVLLLSLVITLYCKRGRKKLLYIFKQPFMRPV | |
| | QTTQEEDGCSCRFPEEEEGGCELRVKFSRSADAPAYQQGQN | |
| | QLYNELNLGRREEYDVLDKRRGRDPEMGGKPRRKNPQEGL | |
| | YNELQKDKMAEAYSEIGMKGERRRGKGHDGLYQGLSTATK | |
| | DTYDALHMQALPPR | |
| 154 | DIVLTQSPASLAVSLGERATINCRASESVSVIGAHLIHWYQQK | anti-BCMA CAR |
| | PGQPPKLLIYLASNLETGVPARFSGSGSGTDFTLTISSLQAED | |
| | AAIYYCLQSRIFPRTFGQGTKLEIKGSTSGSGKPGSGEGSTKG | |
| | QVQLVQSGSELKKPGASVKVSCKASGYTFTDYSINWVRQAP | |
| | GQGLEWMGWINTETREPAYAYDFRGRFVFSLDTSVSTAYLQ | |
| | ISSLKAEDTAVYYCARDYSYAMDYWGQGTLVTVSSAAADT | |
| | GLYICKVELMYPPPYYLGIGNGTQIYVIDPEPCPDSDFLLWIL | |
| | AAVSSGLFFYSFLLTAVSKRGRKKLLYIFKQPFMRPVQTTQE | |
| | EDGCSCRFPEEEEGGCELRVKFSRSADAPAYQQGQNQLYNE | |
| | LNLGRREEYDVLDKRRGRDPEMGGKPRRKNPQEGLYNELQ | |
| | KDKMAEAYSEIGMKGERRRGKGHDGLYQGLSTATKDTYDA | |
| | LHMQALPPR | |
| 155 | DIVLTQSPASLAVSLGERATINCRASESVSVIGAHLIHWYQQK | anti-BCMA CAR |
| | PGQPPKLLIYLASNLETGVPARFSGSGSGTDFTLTISSLQAED | |
| | AAIYYCLQSRIFPRTFGQGTKLEIKGSTSGSGKPGSGEGSTKG | |
| | QVQLVQSGSELKKPGASVKVSCKASGYTFTDYSINWVRQAP | |
| | GQGLEWMGWINTETREPAYAYDFRGRFVFSLDTSVSTAYLQ | |
| | ISSLKAEDTAVYYCARDYSYAMDYWGQGTLVTVSSAAAQI | |
| | KESLRAELRVTERRAEVPTAHPSPSPRPAGQFQTLVVGVVGG | |
| | LLGSLVLLVWVLAVICSKRGRKKLLYIFKQPFMRPVQTTQEE | |
| | DGCSCRFPEEEEGGCELRVKFSRSADAPAYQQGQNQLYNEL | |
| | NLGRREEYDVLDKRRGRDPEMGGKPRRKNPQEGLYNELQK | |
| | DKMAEAYSEIGMKGERRRGKGHDGLYQGLSTATKDTYDAL | |
| | HMQALPPR | |
| 156 | EVQLVESGGGLVKPGGSLRLSCAASGFTFSDYYMSWIRQAP | anti-BCMA CAR |
| | GKGLEWVSYISSSGSTIYYADSVKGRFTISRDNAKNSLYLQM | |
| | NSLRAEDTAVYYCAKVDGDYTEDYWGQGTLVTVSSGGGGS | |
| | GGGGSGGGGSQSALTQPASVSGSPGQSITISCTGSSSDVGKY | |
| | NLVSWYQQPPGKAPKLIIYDVNKRPSGVSNRFSGSKSGNTAT | |
| | LTISGLQGDDEADYYCSSYGGSRSYVFGTGTKVTVLESKYGP | |
| | PCPPCPAPPVAGPSVFLFPPKPKDTLMISRTPEVTCVVVDVSQ | |
| | EDPEVQFNWYVDGVEVHNAKTKPREEQFQSTYRVVSVLTV | |
| | LHQDWLNGKEYKCKVSNKGLPSSIEKTISKAKGQPREPQVY | |
| | TLPPSQEEMTKNQVSLTCLVKGFYPSDIAVEWESNGQPENNY | |
| | KTTPPVLDSDGSFFLYSRLTVDKSRWQEGNVFSCSVMHEAL | |
| | HNHYTQKSLSLSLGKMFWVLVVVGGVLACYSLLVTVAFIIF | |
| | WVKRGRKKLLYIFKQPFMRPVQTTQEEDGCSCRFPEEEEGG | |
| | CELRVKFSRSADAPAYQQGQNQLYNELNLGRREEYDVLDK | |
| | RRGRDPEMGGKPRRKNPQEGLYNELQKDKMAEAYSEIGMK | |
| | GERRRGKGHDGLYQGLSTATKDTYDALHMQALPPR | |
| 157 | EVQLVQSGGGLVQPGRSLRLSCTASGFTFGDYAMSWFKQAP | anti-BCMA CAR |
| | GKGLEWVGFIRSKAYGGTTEYAASVKGRFTISRDDSKSIAYL | |
| | QMNSLKTEDTAVYYCAAWSAPTDYWGQGTLVTVSSGGGGS | |
| | GGGGSGGGGSDIQMTQSPAFLSASVGDRVTVTCRASQGISN | |
| | YLAWYQQKPGNAPRLLIYSASTLQSGVPSRFRGTGYGTEFSL | |
| | TIDSLQPEDFATYYCQQSYTSRQTFGPGTRLDIKESKYGPPCP | |
| | PCPAPPVAGPSVFLFPPKPKDTLMISRTPEVTCVVVDVSQEDP | |
| | EVQFNWYVDGVEVHNAKTKPREEQFQSTYRVVSVLTVLHQ | |
| | DWLNGKEYKCKVSNKGLPSSIEKTISKAKGQPREPQVYTLPP | |
| | SQEEMTKNQVSLTCLVKGFYPSDIAVEWESNGQPENNYKTT | |
| | PPVLDSDGSFFLYSRLTVDKSRWQEGNVFSCSVMHEALHNH | |
| | YTQKSLSLSLGKMFWVLVVVGGVLACYSLLVTVAFIIFWVK | |
| | RGRKKLLYIFKQPFMRPVQTTQEEDGCSCRFPEEEEGGCELR | |
| | VKFSRSADAPAYQQGQNQLYNELNLGRREEYDVLDKRRGR | |
| | DPEMGGKPRRKNPQEGLYNELQKDKMAEAYSEIGMKGERR | |
| | RGKGHDGLYQGLSTATKDTYDALHMQALPPR | |
| 158 | EVQLVESGGGLVKPGGSLRLSCAASGFTFSDYYMSWIRQAP | anti-BCMA CAR |
| | GKGLEWVSYISSSGSTIYYADSVKGRFTISRDNAKNSLYLQM | |
| | NSLRAEDTAVYYCAKVDGPPSFDIWGQGTMVTVSSGGGGS | |
| | GGGGSGGGGSSYVLTQPPSVSVAPGQTARITCGANNIGSKSV | |
| | HWYQQKPGQAPMLVVYDDDDRPSGIPERFSGSNSGNTATLT | |
| | ISGVEAGDEADYFCHLWDRSRDHYVFGTGTKLTVLESKYGP | |
| | PCPPCPAPPVAGPSVFLFPPKPKDTLMISRTPEVTCVVVDVSQ | |
| | EDPEVQFNWYVDGVEVHNAKTKPREEQFQSTYRVVSVLTV | |
| | LHQDWLNGKEYKCKVSNKGLPSSIEKTISKAKGQPREPQVY | |
| | TLPPSQEEMTKNQVSLTCLVKGFYPSDIAVEWESNGQPENNY | |
| | KTTPPVLDSDGSFFLYSRLTVDKSRWQEGNVFSCSVMHEAL | |
| | HNHYTQKSLSLSLGKMFWVLVVVGGVLACYSLLVTVAFIIF | |
| | WVKRGRKKLLYIFKQPFMRPVQTTQEEDGCSCRFPEEEEGG | |
| | CELRVKFSRSADAPAYQQGQNQLYNELNLGRREEYDVLDK | |
| | RRGRDPEMGGKPRRKNPQEGLYNELQKDKMAEAYSEIGMK | |
| | GERRRGKGHDGLYQGLSTATKDTYDALHMQALPPR | |
| 159 | SYELTQPPSASGTPGQRVTMSCSGTSSNIGSHSVNWYQQLPG | anti-BCMA CAR |
| | TAPKLLIYTNNQRPSGVPDRFSGSKSGTSASLAISGLQSEDEA | |
| | DYYCAAWDGSLNGLVFGGGTKLTVLGSRGGGGSGGGGSGG | |
| | GGSLEMAEVQLVQSGAEVKKPGESLKISCKGSGYSFTSYWIG | |
| | WVRQMPGKGLEWMGIIYPGDSDTRYSPSFQGHVTISADKSIS | |
| | TAYLQWSSLKASDTAMYYCARYSGSFDNWGQGTLVTVSSE | |
| | SKYGPPCPPCPAPPVAGPSVFLFPPKPKDTLMISRTPEVTCVV | |
| | VDVSQEDPEVQFNWYVDGVEVHNAKTKPREEQFQSTYRVV | |
| | SVLTVLHQDWLNGKEYKCKVSNKGLPSSIEKTISKAKGQPRE | |
| | PQVYTLPPSQEEMTKNQVSLTCLVKGFYPSDIAVEWESNGQP | |
| | ENNYKTTPPVLDSDGSFFLYSRLTVDKSRWQEGNVFSCSVM | |
| | HEALHNHYTQKSLSLSLGKMFWVLVVVGGVLACYSLLVTV | |
| | AFIIFWVKRGRKKLLYIFKQPFMRPVQTTQEEDGCSCRFPEEE | |
| | EGGCELRVKFSRSADAPAYQQGQNQLYNELNLGRREEYDVL | |
| | DKRRGRDPEMGGKPRRKNPQEGLYNELQKDKMAEAYSEIG | |
| | MKGERRRGKGHDGLYQGLSTATKDTYDALHMQALPPR | |
| 160 | QSALTQPASVSASPGQSIAISCTGTSSDVGWYQQHPGKAPKL | anti-BCMA CAR |
| | MIYEDSKRPSGVSNRFSGSKSGNTASLTISGLQAEDEADYYC | |
| | SSNTRSSTLVFGGGTKLTVLGSRGGGGSGGGGSGGGGSLEM | |
| | AEVQLVQSGAEMKKPGASLKLSCKASGYTFIDYYVYWMRQ | |
| | APGQGLESMGWINPNSGGTNYAQKFQGRVTMTRDTSISTAY | |
| | MELSRLRSDDTAMYYCARSQRDGYMDYWGQGTLVTVSSES | |
| | KYGPPCPPCPAPPVAGPSVFLFPPKPKDTLMISRTPEVTCVVV | |
| | DVSQEDPEVQFNWYVDGVEVHNAKTKPREEQFQSTYRVVS | |
| | VLTVLHQDWLNGKEYKCKVSNKGLPSSIEKTISKAKGQPREP | |
| | QVYTLPPSQEEMTKNQVSLTCLVKGFYPSDIAVEWESNGQPE | |
| | NNYKTTPPVLDSDGSFFLYSRLTVDKSRWQEGNVFSCSVMH | |
| | EALHNHYTQKSLSLSLGKMFWVLVVVGGVLACYSLLVTVA | |
| | FIIFWVKRGRKKLLYIFKQPFMRPVQTTQEEDGCSCRFPEEEE | |
| | GGCELRVKFSRSADAPAYQQGQNQLYNELNLGRREEYDVL | |
| | DKRRGRDPEMGGKPRRKNPQEGLYNELQKDKMAEAYSEIG | |
| | MKGERRRGKGHDGLYQGLSTATKDTYDALHMQALPPR | |
| 161 | QSALTQPASVSASPGQSIAISCTGTSSDVGWYQQHPGKAPKL | anti-BCMA CAR |
| | MIYEDSKRPSGVSNRFSGSKSGNTASLTISGLQAEDEADYYC | |
| | SSNTRSSTLVFGGGTKLTVLGSRGGGGSGGGGSGGGGSLEM | |
| | AEVQLVQSGAEMKKPGASLKLSCKASGYTFIDYYVYWMRQ | |
| | APGQGLESMGWINPNSGGTNYAQKFQGRVTMTRDTSISTAY | |
| | MELSRLRSDDTAMYYCARSQRDGYMDYWGQGTLVTVSSES | |
| | KYGPPCPPCPAPPVAGPSVFLFPPKPKDTLMISRTPEVTCVVV | |
| | DVSQEDPEVQFNWYVDGVEVHNAKTKPREEQFQSTYRVVS | |
| | VLTVLHQDWLNGKEYKCKVSNKGLPSSIEKTISKAKGQPREP | |
| | QVYTLPPSQEEMTKNQVSLTCLVKGFYPSDIAVEWESNGQPE | |
| | NNYKTTPPVLDSDGSFFLYSRLTVDKSRWQEGNVFSCSVMH | |
| | EALHNHYTQKSLSLSLGKMFWVLVVVGGVLACYSLLVTVA | |
| | FIIFWVRSKRSRLLHSDYMNMTPRRPGPTRKHYQPYAPPRDF | |
| | AAYRSRVKFSRSADAPAYQQGQNQLYNELNLGRREEYDVL | |
| | DKRRGRDPEMGGKPRRKNPQEGLYNELQKDKMAEAYSEIG | |
| | MKGERRRGKGHDGLYQGLSTATKDTYDALHMQALPPR | |
| 162 | EVQLVESGGGLVQPGGSLRLSCAVSGFALSNHGMSWVRRAP | anti-BCMA CAR |
| | GKGLEWVSGIVYSGSTYYAASVKGRFTISRDNSRNTLYLQM | |
| | NSLRPEDTAIYYCSAHGGESDVWGQGTTVTVSSASGGGGSG | |
| | GRASGGGGSDIQLTQSPSSLSASVGDRVTITCRASQSISSYLN | |
| | WYQQKPGKAPKLLIYAASSLQSGVPSRFSGSGSGTDFTLTISS | |
| | LQPEDFATYYCQQSYSTPYTFGQGTKVEIKTTTPAPRPPTPAP | |
| | TIASQPLSLRPEACRPAAGGAVHTRGLDFACDIYIWAPLAGT | |
| | CGVLLLSLVITLYCKRGRKKLLYIFKQPFMRPVQTTQEEDGC | |
| | SCRFPEEEEGGCELRVKFSRSADAPAYKQGQNQLYNELNLG | |
| | RREEYDVLDKRRGRDPEMGGKPRRKNPQEGLYNELQKDKM | |
| | AEAYSEIGMKGERRRGKGHDGLYQGLSTATKDTYDALHMQ | |
| | ALPPR | |
| 163 | QVQLVESGGGLVQPGRSLRLSCAASGFTFSNYAMSWVRQAP | anti-BCMA CAR |
| | GKGLGWVSGISRSGENTYYADSVKGRFTISRDNSKNTLYLQ | |
| | MNSLRDEDTAVYYCARSPAHYYGGMDVWGQGTTVTVSSA | |
| | SGGGGSGGRASGGGGSDIVLTQSPGTLSLSPGERATLSCRAS | |
| | QSISSSFLAWYQQKPGQAPRLLIYGASRRATGIPDRFSGSGSG | |
| | TDFTLTISRLEPEDSAVYYCQQYHSSPSWTFGQGTKLEIKTTT | |
| | PAPRPPTPAPTIASQPLSLRPEACRPAAGGAVHTRGLDFACDI | |
| | YIWAPLAGTCGVLLLSLVITLYCKRGRKKLLYIFKQPFMRPV | |
| | QTTQEEDGCSCRFPEEEEGGCELRVKFSRSADAPAYKQGQN | |
| | QLYNELNLGRREEYDVLDKRRGRDPEMGGKPRRKNPQEGL | |
| | YNELQKDKMAEAYSEIGMKGERRRGKGHDGLYQGLSTATK | |
| | DTYDALHMQALPPR | |
| 164 | QVQLVESGGGLVQPGGSLRLSCAVSGFALSNHGMSWVRRA | anti-BCMA CAR |
| | PGKGLEWVSGIVYSGSTYYAASVKGRFTISRDNSRNTLYLQ | |
| | MNSLRPEDTAIYYCSAHGGESDVWGQGTTVTVSSASGGGGS | |
| | GGRASGGGGSDIRLTQSPSPLSASVGDRVTITCQASEDINKFL | |
| | NWYHQTPGKAPKLLIYDASTLQTGVPSRFSGSGSGTDFTLTI | |
| | NSLQPEDIGTYYCQQYESLPLTFGGGTKVEIKTTTPAPRPPTP | |
| | APTIASQPLSLRPEACRPAAGGAVHTRGLDFACDIYIWAPLA | |
| | GTCGVLLLSLVITLYCKRGRKKLLYIFKQPFMRPVQTTQEED | |
| | GCSCRFPEEEEGGCELRVKFSRSADAPAYKQGQNQLYNELN | |
| | LGRREEYDVLDKRRGRDPEMGGKPRRKNPQEGLYNELQKD | |
| | KMAEAYSEIGMKGERRRGKGHDGLYQGLSTATKDTYDALH | |
| | MQALPPR | |
| 165 | EVQLVESGGGLVQPGGSLRLSCAVSGFALSNHGMSWVRRAP | anti-BCMA CAR |
| | GKGLEWVSGIVYSGSTYYAASVKGRFTISRDNSRNTLYLQM | |
| | NSLRPEDTAIYYCSAHGGESDVWGQGTTVTVSSASGGGGSG | |
| | GRASGGGGSEIVLTQSPGTLSLSPGERATLSCRASQSIGSSSLA | |
| | WYQQKPGQAPRLLMYGASSRASGIPDRFSGSGSGTDFTLTIS | |
| | RLEPEDFAVYYCQQYAGSPPFTFGQGTKVEIKTTTPAPRPPTP | |
| | APTIASQPLSLRPEACRPAAGGAVHTRGLDFACDIYIWAPLA | |
| | GTCGVLLLSLVITLYCKRGRKKLLYIFKQPFMRPVQTTQEED | |
| | GCSCRFPEEEEGGCELRVKFSRSADAPAYKQGQNQLYNELN | |
| | LGRREEYDVLDKRRGRDPEMGGKPRRKNPQEGLYNELQKD | |
| | KMAEAYSEIGMKGERRRGKGHDGLYQGLSTATKDTYDALH | |
| | MQALPPR | |
| 166 | QIQLVQSGPDLKKPGETVKLSCKASGYTFTNFGMNWVKQAP | anti-BCMA CAR |
| | GKGFKWMAWINTYTGESYFADDFKGRFAFSVETSATTAYLQ | |
| | INNLKTEDTATYFCARGEIYYGYDGGFAYWGQGTLVTVSAG | |
| | GGGSGGGGSGGGGSDVVMTQSHRFMSTSVGDRVSITCRASQ | |
| | DVNTAVSWYQQKPGQSPKLLIFSASYRYTGVPDRFTGSGSG | |
| | ADFTLTISSVQAEDLAVYYCQQHYSTPWTFGGGTKLDIKTTT | |
| | PAPRPPTPAPTIASQPLSLRPEACRPAAGGAVHTRGLDFACDI | |
| | YIWAPLAGTCGVLLLSLVITLYCKRGRKKLLYIFKQPFMRPV | |
| | QTTQEEDGCSCRFPEEEEGGCELRVKFSRSADAPAYKQGQN | |
| | QLYNELNLGRREEYDVLDKRRGRDPEMGGKPRRKNPQEGL | |
| | YNELQKDKMAEAYSEIGMKGERRRGKGHDGLYQGLSTATK | |
| | DTYDALHMQALPPR | |
| 167 | QIQLVQSGPELKKPGETVKISCKASGYTFTDYSINWVKRAPG | anti-BCMA CAR |
| | KGLKWMGWINTETREPAYAYDFRGRFAFSLETSASTAYLQI | |
| | NNLKYEDTATYFCALDYSYAMDYWGQGTSVTVSSGGGGSG | |
| | GGGSGGGGSQIQLVQSGPELKKPGETVKISCKASGYTFTDYSI | |
| | NWVKRAPGKGLKWMGWINTETREPAYAYDFRGRFAFSLET | |
| | SASTAYLQINNLKYEDTATYFCALDYSYAMDYWGQGTSVT | |
| | VSSTTTPAPRPPTPAPTIASQPLSLRPEACRPAAGGAVHTRGL | |
| | DFACDIYIWAPLAGTCGVLLLSLVITLYCKRGRKKLLYIFKQP | |
| | FMRPVQTTQEEDGCSCRFPEEEEGGCELRVKFSRSADAPAYK | |
| | QGQNQLYNELNLGRREEYDVLDKRRGRDPEMGGKPRRKNP | |
| | QEGLYNELQKDKMAEAYSEIGMKGERRRGKGHDGLYQGLS | |
| | TATKDTYDALHMQALPPR | |
| 168 | QIQLVQSGPELKKPGETVKISCKASGYTFTDYSINWVKRAPG | anti-BCMA CAR |
| | KGLKWMGWINTETREPAYAYDFRGRFAFSLETSASTAYLQI | |
| | NNLKYEDTATYFCALDYSYAMDYWGQGTSVTVSSGGGGSG | |
| | GGGSGGGGSDIVLTQSPASLAMSLGKRATISCRASESVSVIGA | |
| | HLIHWYQQKPGQPPKLLIYLASNLETGVPARFSGSGSGTDFT | |
| | LTIDPVEEDDVAIYSCLQSRIFPRTFGGGTKLEIKTTTPAPRPP | |
| | TPAPTIASQPLSLRPEACRPAAGGAVHTRGLDFACDIYIWAPL | |
| | AGTCGVLLLSLVITLYCKRGRKKLLYIFKQPFMRPVQTTQEE | |
| | DGCSCRFPEEEEGGCELRVKFSRSADAPAYKQGQNQLYNEL | |
| | NLGRREEYDVLDKRRGRDPEMGGKPRRKNPQEGLYNELQK | |
| | DKMAEAYSEIGMKGERRRGKGHDGLYQGLSTATKDTYDAL | |
| | HMQALPPR | |
| 169 | QIQLVQSGPELKKPGETVKISCKASGYTFRHYSMNWVKQAP | anti-BCMA CAR |
| | GKGLKWMGRINTESGVPIYADDFKGRFAFSVETSASTAYLVI | |
| | NNLKDEDTASYFCSNDYLYSLDFWGQGTALTVSSGGGGSGG | |
| | GGSGGGGSDIVLTQSPPSLAMSLGKRATISCRASESVTILGSH | |
| | LIYWYQQKPGQPPTLLIQLASNVQTGVPARFSGSGSRTDFTL | |
| | TIDPVEEDDVAVYYCLQSRTIPRTFGGGTKLEIKTTTPAPRPP | |
| | TPAPTIASQPLSLRPEACRPAAGGAVHTRGLDFACDIYIWAPL | |
| | AGTCGVLLLSLVITLYCKRGRKKLLYIFKQPFMRPVQTTQEE | |
| | DGCSCRFPEEEEGGCELRVKFSRSADAPAYKQGQNQLYNEL | |
| | NLGRREEYDVLDKRRGRDPEMGGKPRRKNPQEGLYNELQK | |
| | DKMAEAYSEIGMKGERRRGKGHDGLYQGLSTATKDTYDAL | |
| | HMQALPPR | |
| 170 | QIQLVQSGPELKKPGETVKISCKASGYTFTHYSMNWVKQAP | anti-BCMA CAR |
| | GKGLKWMGRINTETGEPLYADDFKGRFAFSLETSASTAYLVI | |
| | NNLKNEDTATFFCSNDYLYSCDYWGQGTTLTVSSGGGGSGG | |
| | GGSGGGGSDIVLTQSPPSLAMSLGKRATISCRASESVTILGSH | |
| | LIYWYQQKPGQPPTLLIQLASNVQTGVPARFSGSGSRTDFTL | |
| | TIDPVEEDDVAVYYCLQSRTIPRTFGGGTKLEIKTTTPAPRPP | |
| | TPAPTIASQPLSLRPEACRPAAGGAVHTRGLDFACDIYIWAPL | |
| | AGTCGVLLLSLVITLYCKRGRKKLLYIFKQPFMRPVQTTQEE | |
| | DGCSCRFPEEEEGGCELRVKFSRSADAPAYKQGQNQLYNEL | |
| | NLGRREEYDVLDKRRGRDPEMGGKPRRKNPQEGLYNELQK | |
| | DKMAEAYSEIGMKGERRRGKGHDGLYQGLSTATKDTYDAL | |
| | HMQALPPR | |
| 171 | DIVLTQSPPSLAMSLGKRATISCRASESVTILGSHLIHWYQQK | anti-BCMA CAR |
| | PGQPPTLLIQLASNVQTGVPARFSGSGSRTDFTLTIDPVEEDD | |
| | VAVYYCLQSRTIPRTFGGGTKLEIKGSTSGSGKPGSGEGSTK | |
| | GQIQLVQSGPELKKPGETVKISCKASGYTFTDYSINWVKRAP | |
| | GKGLKWMGWINTETREPAYAYDFRGRFAFSLETSASTAYLQ | |
| | INNLKYEDTATYFCALDYSYAMDYWGQGTSVTVSSFVPVFL | |
| | PAKPTTTPAPRPPTPAPTIASQPLSLRPEACRPAAGGAVHTRG | |
| | LDFACDIYIWAPLAGTCGVLLLSLVITLYCNHRNRSKRSRLL | |
| | HSDYMNMTPRRPGPTRKHYQPYAPPRDFAAYRSRVKFSRSA | |
| | DAPAYQQGQNQLYNELNLGRREEYDVLDKRRGRDPEMGGK | |
| | PRRKNPQEGLYNELQKDKMAEAYSEIGMKGERRRGKGHDG | |
| | LYQGLSTATKDTYDALHMQALPPR | |
| 172 | QVQLVQSGAEVKKPGASVKVSCKASGYSFPDYYINWVRQA | anti-BCMA CAR |
| | PGQGLEWMGWIYFASGNSEYNQKFTGRVTMTRDTSINTAY | |
| | MELSSLTSEDTAVYFCASLYDYDWYFDVWGQGTMVTVSSG | |
| | GGGSGGGGSGGGGSDIVMTQTPLSLSVTPGQPASISCKSSQSL | |
| | VHSNGNTYLHWYLQKPGQSPQLLIYKVSNRFSGVPDRFSGS | |
| | GSGTDFTLKISRVEAEDVGIYYCSQSSIYPWTFGQGTKLEIKG | |
| | LAVSTISSFFPPGYQIYIWAPLAGTCGVLLLSLVITLYCKRGR | |
| | KKLLYIFKQPFMRPVQTTQEEDGCSCRFPEEEEGGCELRVKF | |
| | SRSADAPAYQQGQNQLYNELNLGRREEYDVLDKRRGRDPE | |
| | MGGKPRRKNPQEGLYNELQKDKMAEAYSEIGMKGERRRGK | |
| | GHDGLYQGLSTATKDTYDALHMQALPPR | |
| 173 | QVQLVQSGAEVKKPGASVKVSCKASGYSFPDYYINWVRQA | anti-BCMA CAR |
| | PGQGLEWMGWIYFASGNSEYNQKFTGRVTMTRDTSINTAY | |
| | MELSSLTSEDTAVYFCASLYDYDWYFDVWGQGTMVTVSSG | |
| | GGGSGGGGSGGGGSDIVMTQTPLSLSVTPGQPASISCKSSQSL | |
| | VHSNGNTYLHWYLQKPGQSPQLLIYKVSNRFSGVPDRFSGS | |
| | GSGTDFTLKISRVEAEDVGIYYCSQSSIYPWTFGQGTKLEIKT | |
| | TTPAPRPPTPAPTIASQPLSLRPEACRPAAGGAVHTRGLDFAC | |
| | DIYIWAPLAGTCGVLLLSLVITLYCKRGRKKLLYIFKQPFMRP | |
| | VQTTQEEDGCSCRFPEEEEGGCELRVKFSRSADAPAYQQGQ | |
| | NQLYNELNLGRREEYDVLDKRRGRDPEMGGKPRRKNPQEG | |
| | LYNELQKDKMAEAYSEIGMKGERRRGKGHDGLYQGLSTAT | |
| | KDTYDALHMQALPPR | |
| 174 | QVQLVQSGAEVKKPGASVKVSCKASGYSFPDYYINWVRQA | anti-BCMA CAR |
| | PGQGLEWMGWIYFASGNSEYNQKFTGRVTMTRDTSINTAY | |
| | MELSSLTSEDTAVYFCASLYDYDWYFDVWGQGTMVTVSSG | |
| | GGGSGGGGSGGGGSDIVMTQTPLSLSVTPGQPASISCKSSQSL | |
| | VHSNGNTYLHWYLQKPGQSPQLLIYKVSNRFSGVPDRFSGS | |
| | GSGTDFTLKISRVEAEDVGIYYCSQSSIYPWTFGQGTKLEIKE | |
| | PKSPDKTHTCPPCPAPPVAGPSVFLFPPKPKDTLMIARTPEVT | |
| | CVVVDVSHEDPEVKFNWYVDGVEVHNAKTKPREEQYNSTY | |
| | RVVSVLTVLHQDWLNGKEYKCKVSNKALPAPIEKTISKAKG | |
| | QPREPQVYTLPPSRDELTKNQVSLTCLVKGFYPSDIAVEWES | |
| | NGQPENNYKTTPPVLDSDGSFFLYSKLTVDKSRWQQGNVFS | |
| | CSVMHEALHNHYTQKSLSLSPGKIYIWAPLAGTCGVLLLSLV | |
| | ITLYCKRGRKKLLYIFKQPFMRPVQTTQEEDGCSCRFPEEEEG | |
| | GCELRVKFSRSADAPAYQQGQNQLYNELNLGRREEYDVLD | |
| | KRRGRDPEMGGKPRRKNPQEGLYNELQKDKMAEAYSEIGM | |
| | KGERRRGKGHDGLYQGLSTATKDTYDALHMQALPPR | |
| 175 | QVQLVQSGAEVKKPGASVKVSCKASGYSFPDYYINWVRQA | anti-BCMA CAR |
| | PGQGLEWMGWIYFASGNSEYNQKFTGRVTMTRDTSSSTAY | |
| | MELSSLRSEDTAVYFCASLYDYDWYFDVWGQGTMVTVSSG | |
| | GGGSGGGGSGGGGSDIVMTQTPLSLSVTPGEPASISCKSSQSL | |
| | VHSNGNTYLHWYLQKPGQSPQLLIYKVSNRFSGVPDRFSGS | |
| | GSGADFTLKISRVEAEDVGVYYCAETSHVPWTFGQGTKLEIK | |
| | GLAVSTISSFFPPGYQIYIWAPLAGTCGVLLLSLVITLYCKRG | |
| | RKKLLYIFKQPFMRPVQTTQEEDGCSCRFPEEEEGGCELRVK | |
| | FSRSADAPAYQQGQNQLYNELNLGRREEYDVLDKRRGRDPE | |
| | MGGKPRRKNPQEGLYNELQKDKMAEAYSEIGMKGERRRGK | |
| | GHDGLYQGLSTATKDTYDALHMQALPPR | |
| 176 | QVQLVQSGAEVKKPGASVKVSCKASGYSFPDYYINWVRQA | anti-BCMA CAR |
| | PGQGLEWMGWIYFASGNSEYNQKFTGRVTMTRDTSSSTAY | |
| | MELSSLRSEDTAVYFCASLYDYDWYFDVWGQGTMVTVSSG | |
| | GGGSGGGGSGGGGSDIVMTQTPLSLSVTPGEPASISCKSSQSL | |
| | VHSNGNTYLHWYLQKPGQSPQLLIYKVSNRFSGVPDRFSGS | |
| | GSGADFTLKISRVEAEDVGVYYCAETSHVPWTFGQGTKLEIK | |
| | TTTPAPRPPTPAPTIASQPLSLRPEACRPAAGGAVHTRGLDFA | |
| | CDIYIWAPLAGTCGVLLLSLVITLYCKRGRKKLLYIFKQPFM | |
| | RPVQTTQEEDGCSCRFPEEEEGGCELRVKFSRSADAPAYQQG | |
| | QNQLYNELNLGRREEYDVLDKRRGRDPEMGGKPRRKNPQE | |
| | GLYNELQKDKMAEAYSEIGMKGERRRGKGHDGLYQGLSTA | |
| | TKDTYDALHMQALPPR | |
| 177 | QVQLVQSGAEVKKPGASVKVSCKASGYSFPDYYINWVRQA | anti-BCMA CAR |
| | PGQGLEWMGWIYFASGNSEYNQKFTGRVTMTRDTSSSTAY | |
| | MELSSLRSEDTAVYFCASLYDYDWYFDVWGQGTMVTVSSG | |
| | GGGSGGGGSGGGGSDIVMTQTPLSLSVTPGEPASISCKSSQSL | |
| | VHSNGNTYLHWYLQKPGQSPQLLIYKVSNRFSGVPDRFSGS | |
| | GSGADFTLKISRVEAEDVGVYYCAETSHVPWTFGQGTKLEIK | |
| | EPKSPDKTHTCPPCPAPPVAGPSVFLFPPKPKDTLMIARTPEV | |
| | TCVVVDVSHEDPEVKFNWYVDGVEVHNAKTKPREEQYNST | |
| | YRVVSVLTVLHQDWLNGKEYKCKVSNKALPAPIEKTISKAK | |
| | GQPREPQVYTLPPSRDELTKNQVSLTCLVKGFYPSDIAVEWE | |
| | SNGQPENNYKTTPPVLDSDGSFFLYSKLTVDKSRWQQGNVF | |
| | SCSVMHEALHNHYTQKSLSLSPGKIYIWAPLAGTCGVLLLSL | |
| | VITLYCKRGRKKLLYIFKQPFMRPVQTTQEEDGCSCRFPEEEE | |
| | GGCELRVKFSRSADAPAYQQGQNQLYNELNLGRREEYDVL | |
| | DKRRGRDPEMGGKPRRKNPQEGLYNELQKDKMAEAYSEIG | |
| | MKGERRRGKGHDGLYQGLSTATKDTYDALHMQALPPR | |
| 178 | IYIWAPLAGTCGVLLLSLVITLYCNHRN | CD8a TM |
| 179 | IYIWAPLAGTCGVLLLSLVIT | CD8a TM |
| 180 | RAAA | Linking peptide |
| 181 | EVQLVESGGGLVKPGGSLRLSCAASGFTFSDYYMSWIRQAP | Variable heavy |
| | GKGLEWVSYISSSGSTIYYADSVKGRFTISRDNAKNSLYLQM | (VH) Anti- |
| | NSLRAEDTAVYYCAKVDGDYTEDYWGQGTLVTVSS | BCMA |
| 182 | QSALTQPASVSGSPGQSITISCTGSSSDVGKYNLVSWYQQPPG | Variable light |
| | KAPKLIIYDVNKRPSGVSNRFSGSKSGNTATLTISGLQGDDEA | (VL) Anti- |
| | DYYCSSYGGSRSYVFGTGTKVTVL | BCMA |
| 183 | EVQLVQSGGGLVQPGRSLRLSCTASGFTFGDYAMSWFRQAP | Variable heavy |
| | GKGLEWVGFIRSKAYGGTTEYAASVKGRFTISRDDSKSIAYL | (VH) Anti- |
| | QMNSLKTEDTAVYYCAAWSAPTDYWGQGTLVTVSS | BCMA |
| 184 | DIQMTQSPAFLSASVGDRVTVTCRASQGISNYLAWYQQKPG | Variable light |
| | NAPRLLIYSASTLQSGVPSRFRGTGYGTEFSLTIDSLQPEDFAT | (VL) Anti- |
| | YYCQQSYTSRQTFGPGTRLDIK | BCMA |
| 185 | EVQLVESGGGLVKPGGSLRLSCAASGFTFSDYYMSWIRQAP | Variable heavy |
| | GKGLEWVSYISSSGSTIYYADSVKGRFTISRDNAKNSLYLQM | (VH) Anti- |
| | NSLRAEDTAVYYCAKVDGPPSFDIWGQGTMVTVSS | BCMA |
| 186 | SYVLTQPPSVSVAPGQTARITCGANNIGSKSVHWYQQKPGQ | Variable light |
| | APMLVVYDDDDRPSGIPERFSGSNSGNTATLTISGVEAGDEA | (VL) Anti- |
| | DYFCHLWDRSRDHYVFGTGTKLTVL | BCMA |
| 187 | EVQLVQSGAEVKKPGSSVKVSCKASGGTFSSYAISWVRQAP | Variable heavy |
| | GQGLEWMGRIIPILGIANYAQKFQGRVTMTEDTSTDTAYME | (VH) Anti- |
| | LSSLRSEDTAVYYCARSGYSKSIVSYMDYWGQGTLVTVSS | BCMA |
| 188 | LPVLTQPPSTSGTPGQRVTVSCSGSSSNIGSNVVFWYQQLPGT | Variable light |
| | APKLVIYRNNQRPSGVPDRFSVSKSGTSASLAISGLRSEDEAD | (VL) Anti- |
| | YYCAAWDDSLSGYVFGTGTKVTVLG | BCMA |
| 189 | ASGGGGSGGRASGGGGS | Linker |
| 190 | MAKERGLISP SDFAQLQKYMEYSTKKVSDV LKLFEDGEMA | DGKα |
| | KYVQGDAIGY EGFQQFLKIY LEVDNVPRHL SLALFQSFET G | |
| | HCLNETNVT KDVVCLNDVS CYFSLLEGGR PEDKLEFTFK L | |
| | YDTDRNGIL DSSEVDKIIL QMMRVAEYLD WDVSELRPIL QE | |
| | MMKEIDYD GSGSVSQAEW VRAGATTVPL LVLLGLEMTL K | |
| | DDGQHMWRP KRFPRPVYCN LCESSIGLGK QGLSCNLCKY T | |
| | VHDQCAMKA LPCEVSTYAK SRKDIGVQSH VWVRGGCESG | |
| | RCDRCQKKIR IYHSLTGLHC VWCHLEIHDD CLQAVGHECD | |
| | CGLLRDHILP PSSIYPSVLA SGPDRKNSKT SQKTMDDLNL ST | |
| | SEALRIDP VPNTHPLLVF VNPKSGGKQG QRVLWKFQYILNP | |
| | RQVFNLL KDGPEIGLRL FKDVPDSRIL VCGGDGTVGW ILET | |
| | IDKANL PVLPPVAVLP LGTGNDLARC LRWGGGYEGQ NLA | |
| | KILKDLE MSKVVHMDRW SVEVIPQQTE EKSDPVPFQI INNY | |
| | FSIGVD ASIAHRFHIM REKYPEKFNS RMKNKLWYFE FATSE | |
| | SIFST CKKLEESLTV EICGKPLDLS NLSLEGIAVL NIPSMHGG | |
| | SN LWGDTRRPHG DIYGINQALG ATAKVITDPD ILKTCVPDL | |
| | S DKRLEVVGLE GAIEMGQIYT KLKNAGRRLA KCSEITFHTT | |
| | KTLPMQIDGE PWMQTPCTIK ITHKNQMPML MGPPPRSTNF | |
| | FGFLSGSSET VRFQGHHHHH H | |
| 191 | MEPRDGSPEA RSSDSESASA SSSGSERDAG PEPDKAPRRL N | DGKζ |
| | KRRFPGLRL FGHRKAITKS GLQHLAPPPP TPGAPCSESERQI | |
| | RSTVDWS ESATYGEHIW FETNVSGDFC YVGEQYCVAR mL | |
| | QKSVSRRK CAACKIVVHT PCIEQLEKIN FRCKPSFRES GSRN | |
| | VREPTF VRHHWVHRRR QDGKCRHCGK GFQQKFTFHS KEI | |
| | VAISCSW CKQAYHSKVS CFMLQQIEEP CSLGVHAAVV IPPT | |
| | WILRAR RPQNTLKASK KKKRASFKRK SSKKGPEEGR WRPF | |
| | IIRPTP SPLMKPLLVF VNPKSGGNQG AKIIQSFLWY LNPRQV | |
| | FDLS QGGPKEALEM YRKVHNLRIL ACGGDGTVGW ILSTLD | |
| | QLRL KPPPPVAILP LGTGNDLART LNWGGGYTDE PVSKILS | |
| | HVE EGNVVQLDRW DLHAEPNPEA GPEDRDEGAT DRLPLD | |
| | VENN YFSLGFDAHV TLEFHESREA NPEKFNSRFR NKMFYA | |
| | GTAF SDFLMGSSKD LAKHIR VVCD GMDLTPKIQD LKPQCV | |
| | VFLN IPRYCAGTMP WGHPGEHHDF EPQRHDDGYL EVIGFT | |
| | MTSL AALQVGGHGE RLTQCREVVL TTSKAIPVQV DGEPCK | |
| | LAAS RIRIALRNQA TMVQKAKRRS AAPLHSDQQP VPEQLRI | |
| | QVS RVSMHDYEAL HYDKEQLKEA SVPLGTVVVP GDSDLE | |
| | LCRA HIERLQQEPD GAGAKSPTCQ KLSPKWCFLD ATTASRF | |
| | YRI DRAQEHLNYV TEIAQDEIYI LDPELLGASA RPDLPTPTS | |
| | P LPTSPCSPTP RSLQGDAAPP QGEELIEAAK RNDFCKLQEL | |
| | HRAGGDLMHR DEQSRTLLHH AVSTGSKDVV RYLLDHAPP | |
| | E ILDAVEENGE TCLHQAAALG QRTICHYIVE AGASLMKTD | |
| | Q QGDTPRQRAE KAQDTELAAY LENRQHYQMI QREDQETA | |
| | VG SSETVRFQGH HHHHH | |
| 192 | KLPQLCTEL | HPV E6(18-26) |
| 193 | TIHDIILECV | HPV E6(29-38) |
| 194 | FAFRDLCIV | HPV E6(52-60) |
| 195 | TLGIVCPI | HPV E7(86-93) |
| 196 | YMLDLQPET | HPV E7(11-19) |
| 197 | GTLGIVCPI | HPV E7(85-93) |
| 198 | LLMGTLGIV | HPV E7(82-90) |
| 199 | TLHEYMLDL | HPV E7(7-15) |
| 200 | atggtgctgaagttttctgtgagcatcctgtggattcagctggcctgggtgtccacccagctcct | TCR 57 Alpha |
| | ggagcagagcccccagttcctgtccatccaggagggcgagaacctgaccgtgtactgcaacagct | Var. + Constant |
| | cttctgtcttttcatcactgcagtggtatagacaagaaccgggtgaaggtccagttctgctggtg | (nt) |
| | accgtcgtcaccggcggcgaggtgaagaagctaaagcgcctgacgttccagttcggagacgcgc | |
| | ggaaggactcgtcgctgcacatcaccgccgcccagcccggcgacaccggcctgtacctgtgc | |
| | gctggcgcgcgcaacttcaacaagttctacttcggcagcggcaccaagctgaacgtgaaaccg | |
| | aatatccagaatccggaccccgcggtatatcaactgcgcgactcaaaatcatccgataagagtgt | |
| | ctgtttgtttactgacttcgacagtcaaactaatgtctctcagagcaaagattccgatgtctaca | |
| | tcactgacaagtgcgttctggatatgcggagcatggattttaagtccaactccgccgtagcctgg | |
| | tccaacaagtcagactttgcctgtgcaaatgctttcaacaactcaattatccctgaggacacttt | |
| | ctttccttcaccggagtcctcatgcgatgttaaactggtcgaaaaatcttttgagacggatacga | |
| | acctcaacttccaaaatttgagcgttattggctttaggattctgcttctcaaggttgcggggttc | |
| | aatctcctgatgacgttgcggctttggagcagctaa | |
| 201 | TQLLEQSPQFLSIQEGENLTVYCNSSSVFSSLQWYRQEPGEGP | TCR 57 Alpha |
| | VLLVTVVTGGEVKKLKRLTFQFGDARKDSSLHITAAQPGDT | Var. + Constant |
| | GLYLCAGARNFNKFYFGSGTKLNVKPNIQNPDPAVYQLRDS | (aa) |
| | KSSDKSVCLFTDFDSQTNVSQSKDSDVYITDKCVLDMRSMD | |
| | FKSNSAVAWSNKSDFACANAFNNSIIPEDTFFPSPESSCDVKL | |
| | VEKSFETDTNLNFQNLSVIGFRILLLKVAGFNLLMTLRLWSS | |
| 202 | TQLLEQSPQFLSIQEGENLTVYCNSSSVFSSLQWYRQEPGEGP | TCR 57 Alpha |
| | VLLVTVVTGGEVKKLKRLTFQFGDARKDSSLHITAAQPGDT | Var. (aa) |
| | GLYLCAGARNFNKFYFGSGTKLNVKP | |
| 203 | SVFSS | TCR 57 Alpha |
| | | CDR1 (aa) |
| 204 | VVTGGEV | TCR 57 Alpha |
| | | CDR2 (aa) |
| 205 | AGARNFNKFY | TCR 57 Alpha |
| | | CDR3 (aa) |
| 206 | atgctcctgctgcttctgctcctcgggcccggcagcggtctcggcgccgtcgtgtcgcagcacc | TCR 57 Beta |
| | cgtcgtgggtgatttgcaagagcggaacgagcgtgaagatcgagtgccgctcgctcgacttcca | Var. + Constant |
| | ggccacaaccatgttctggtaccgccagtttcccaagcagagcctcatgctgatggccacaagc | (nt) |
| | aatgaaggcagcaaggccacttatgaacagggcgtggagaaggacaagtttctgatcaaccac | |
| | gcctccctgaccctgtccaccctcactgtgaccagcgcccaccctgaggacagcagcttctacat | |
| | ctgcagcgcccgcagctggaggggcggcctggagcagttcttcggccctggcacccggctga | |
| | cagtgctggaagatctgaagaacgtcttcccacctgaggtggccgtgttcgagccttctgaggcc | |
| | gagatcagccacacacagaaagccacactcgtgtgtctggccaccggcttctatcccgatcacg | |
| | tggaactgtcttggtgggtcaacggcaaagaggtgcacagcggcgtctgtaccgatcctcagcc | |
| | tctgaaagagcagcccgctctgaacgacagcagatactgcctgagcagcagactgagagtgtc | |
| | cgccaccttctggcagaaccccagaaaccacttcagatgccaggtgcagttctacggcctgagc | |
| | gagaacgatgagtggacccaggatagagccaagcctgtgacacagatcgtgtctgccgaagcc | |
| | tggggcagagccgattgtggctttaccagcgagagctaccagcagggcgtgctgtctgccaca | |
| | atcctgtacgagatcctgctgggcaaagccactctgtacgccgtgctggtgtctgccctggtgct | |
| | gatggccatggtcaagcggaaggatagcagaggc | |
| 207 | GAVVSQHPSWVICKSGTSVKIECRSLDFQATTMFWYRQFPK | TCR 57 Beta |
| | QSLMLMATSNEGSKATYEQGVEKDKFLINHASLTLSTLTVTS | Var. + Constant |
| | AHPEDSSFYICSARSWRGGLEQFFGPGTRLTVLEDLKNVFPPE | (aa) |
| | VAVFEPSEAEISHTQKATLVCLATGFYPDHVELSWWVNGKE | |
| | VHSGVCTDPQPLKEQPALNDSRYCLSSRLRVSATFWQNPRN | |
| | HFRCQVQFYGLSENDEWTQDRAKPVTQIVSAEAWGRADCG | |
| | FTSESYQQGVLSATILYEILLGKATLYAVLVSALVLMAMVKR | |
| | KDSRG | |
| 208 | GAVVSQHPSWVICKSGTSVKIECRSLDFQATTMFWYRQFPK | TCR 57 Beta |
| | QSLMLMATSNEGSKATYEQGVEKDKFLINHASLTLSTLTVTS | Var. (aa) |
| | AHPEDSSFYICSARSWRGGLEQFFGPGTRLTVL | |
| 209 | DFQATT | TCR 57 Beta |
| | | CDR1 (aa) |
| 210 | SNEGSKA | TCR 57 Beta |
| | | CDR2 (aa) |
| 211 | SARSWRGGLEQF | TCR 57 Beta |
| | | CDR3 (aa) |
| 212 | atgctgctgcttctgctgcttctggggccaggctccgggcttggtgctgtcgtctctcaacatcc | TCR 57 Beta- |
| | gagctgggttatctgtaagagtggaacctctgtgaagatcgagtgccgttccctggactttcagg | P2A-Alpha (nt) |
| | ccacaactatgttttggtatcgtcagttcccgaaacagagtctcatgctgatggcaacttccaat | |
| | gagggctccaaggccacatacgagcaaggcgtcgagaaggacaagtttctcatcaaccatgcaag | |
| | cctgaccttgtccactctgacagtgaccagtgcccatcctgaagacagcagcttctacatctgca | |
| | gtgctagatcttggcgggggggccttgagcagttcttcgggccagggacacggctcaccgtgcta | |
| | gaagatctgaagaacgtcttcccacctgaggtggccgtgttcgagccttctgaggccgagatcag | |
| | ccacacacagaaagccacactcgtgtgtctggccaccggcttctatcccgatcacgtggaactgt | |
| | cttggtgggtcaacggcaaagaggtgcacagcggcgtctgtaccgatcctcagcctctgaaagag | |
| | cagcccgctctgaacgacagcagatactgcctgagcagcagactgagagtgtccgccaccttctg | |
| | gcagaaccccagaaaccacttcagatgccaggtgcagttctacggcctgagcgagaacgatga | |
| | gtggacccaggatagagccaagcctgtgacacagatcgtgtctgccgaagcctggggcagag | |
| | ccgattgtggctttaccagcgagagctaccagcagggcgtgctgtctgccacaatcctgtacgag | |
| | atcctgctgggcaaagccactctgtacgccgtgctggtgtctgccctggtgctgatggccatggt | |
| | caagcggaaggatagcagaggcggaagcggcgccacaaacttctcactgctgaaacaggctg | |
| | gcgacgtggaggagaatcctggcccaatggtcctgaaattctccgtgtccattctttggattcag | |
| | ttggcatgggtgagcacccagctgctggagcagagccctcagtttctaagcatccaagagggaga | |
| | aaatctcactgtgtactgcaactcctcaagtgttttttccagcttacaatggtacagacaggagc | |
| | ctggggaaggtcctgtcctcctggtgacagtagttacgggtggagaagtgaagaagctgaagaga | |
| | ctaacctttcagtttggtgatgcaagaaaggacagttctctccacatcactgcagcccagcctgg | |
| | tgatacaggcctctacctctgtgcaggagctcgcaacttcaacaaattttactttggatctggga | |
| | ccaaactcaatgtaaaaccaaatatccagaatccggaccccgcggtatatcaactgcgcgactca | |
| | aaatcatccgataagagtgtctgtttgtttactgacttcgacagtcaaactaatgtctctcagag | |
| | caaagattccgatgtctacatcactgacaagtgcgttctggatatgcggagcatggattttaagt | |
| | ccaactccgccgtagcctggtccaacaagtcagactttgcctgtgcaaatgctttcaacaactca | |
| | attatccctgaggacactttctttccttcaccggagtcctcatgcgatgttaaactggtcgaaaa | |
| | atcttttgagacggatacgaacctcaacttccaaaatttgagcgttattggctttaggattctgc | |
| | ttctcaaggttgcggggttcaatctcctgatgacgttgcggctttggagcagc | |
| 213 | atgaagaaactgctggccatgatcctgtggctgcagctggatagactgagcggcgagctgaag | TCR 37 Alpha |
| | gtggaacagaaccctctgttcctgagcatgcaagagggcaagaactacaccatctactgcaact | Var. + Constant |
| | acagcaccaccagcgaccggctgtactggtacagacaggatccaggcaagagcctggaaagc | (nt) |
| | ctgttcgtgctgctgagcaatggcgccgtgaaacaagagggtagactgatggccagcctggac | |
| | accaaggccagactgagcacactgcacattacagccgccgtgcacgatctgagcgccacctatt | |
| | tttgtgccggctactctggcgccggaagctaccagctgacatttggcaagggcaccaagctgag | |
| | cgtgatcccc | |
| 214 | ELKVEQNPLFLSMQEGKNYTIYCNYSTTSDRLYWYRQDPGK | TCR 37 Alpha |
| | SLESLFVLLSNGAVKQEGRLMASLDTKARLSTLHITAAVHDL | Var. + Constant |
| | SATYFCAGYSGAGSYQLTFGKGTKLSVIPNIQNPDPAVYQLR | (aa) |
| | DSKSSDKSVCLFTDFDSQTNVSQSKDSDVYITDKCVLDMRS | |
| | MDFKSNSAVAWSNKSDFACANAFNNSIIPEDTFFPSPESSCDV | |
| | KLVEKSFETDTNLNFQNLSVIGFRILLLKVAGFNLLMTLRLW | |
| | SS | |
| 215 | ELKVEQNPLFLSMQEGKNYTIYCNYSTTSDRLYWYRQDPGK | TCR 37 Alpha |
| | SLESLFVLLSNGAVKQEGRLMASLDTKARLSTLHITAAVHDL | Var. (aa) |
| | SATYFCAGYSGAGSYQLTFGKGTKLSVIP | |
| 216 | atgggaacaagactgctgtgttgggtcgtgctgggcttcctgggcacagatcatacaggtgccg | TCR 37 Beta |
| | gtgtctctcagagccccagatacaaggtggccaagcgcggacaggatgtggccctgagatgtg | Var. + Constant |
| | atcctatcagcggccacgtgtccctgttctggtatcagcaggctctcggacagggccccgagttc | (nt) |
| | ctgacctactttcagaatgaggcccagctggacaagagcggcctgcctagcgatagattcttcgc | |
| | cgaaagacccgagggcagcgtgtccacactgaagatccagagaacccagcaagaggacagc | |
| | gccgtgtacctgtgtgcctctagtctgctgctgggcgcctacggctacacatttggctctggcac | |
| | cagactgaccgtggtc | |
| 217 | GAGVSQSPRYKVAKRGQDVALRCDPISGHVSLFWYQQALG | TCR 37 Beta |
| | QGPEFLTYFQNEAQLDKSGLPSDRFFAERPEGSVSTLKIQRTQ | Var. + Constant |
| | QEDSAVYLCASSLLLGAYGYTFGSGTRLTVVEDLNKVFPPEV | (aa) |
| | AVFEPSEAEISHTQKATLVCLATGFFPDHVELSWWVNGKEV | |
| | HSGVCTDPQPLKEQPALNDSRYCLSSRLRVSATFWQNPRNH | |
| | FRCQVQFYGLSENDEWTQDRAKPVTQIVSAEAWGRADCGF | |
| | TSVSYQQGVLSATILYEILLGKATLYAVLVSALVLMAMVKR | |
| | KDF | |
| 218 | GAGVSQSPRYKVAKRGQDVALRCDPISGHVSLFWYQQALG | TCR 37 Beta |
| | QGPEFLTYFQNEAQLDKSGLPSDRFFAERPEGSVSTLKIQRTQ | Var. (aa) |
| | QEDSAVYLCASSLLLGAYGYTFGSGTRLTVV | |
| 219 | TTSDR | TCR 37 Alpha |
| | | CDR1 (aa) |
| 220 | LLSNGAV | TCR 37 Alpha |
| | | CDR2 (aa) |
| 221 | AGYSGAGSYQLT | TCR 37 Alpha |
| | | CDR3 (aa) |
| 222 | SGHVS | TCR 37 Beta |
| | | CDR1 (aa) |
| 223 | FQNEAQ | TCR 37 Beta |
| | | CDR2 (aa) |
| 224 | ASSLLLGAYGYT | TCR 37 Beta |
| | | CDR3 (aa) |
| 225 | atgatgaagtccctgcgggtgctgctggtcatcctgtggctgcaactgagctgggtctggtccca | TCR 49 Alpha |
| | gcagaaagaggtggaacagaacagcggccctctgtctgttcctgaaggcgctatcgccagcct | Var. + Constant |
| | gaactgcacctacagcgatagaggcagccagagcttcttctggtacagacagtacagcggcaa | (nt) |
| | gagccccgagctgatcatgttcatctacagcaacggcgacaaagaggacggccggtttacagc | |
| | ccagctgaacaaggccagccagtacgtgtccctgctgatcagagatagccagcctagcgacag | |
| | cgccacctatctgtgcgccgtgaatatcggcaccagcggcacctacaagtacatcttcggcacc | |
| | ggcaccagactgaaggtgctggct | |
| 226 | QKEVEQNSGPLSVPEGAIASLNCTYSDRGSQSFFWYRQYSGK | TCR 49 Alpha |
| | SPELIMFIYSNGDKEDGRFTAQLNKASQYVSLLIRDSQPSDSA | Var. + Constant |
| | TYLCAVNIGTSGTYKYIFGTGTRLKVLANIQNPDPAVYQLRD | (aa) |
| | SKSSDKSVCLFTDFDSQTNVSQSKDSDVYITDKCVLDMRSM | |
| | DFKSNSAVAWSNKS2DFACANAFNNSIIPEDTFFPSPESSCDV | |
| | KLVEKSFETDTNLNFQNLSVIGFRILLLKVAGFNLLMTLRLW | |
| | SS | |
| 227 | QKEVEQNSGPLSVPEGAIASLNCTYSDRGSQSFFWYRQYSGK | TCR 49 Alpha |
| | SPELIMFIYSNGDKEDGRFTAQLNKASQYVSLLIRDSQPSDSA | Var. (aa) |
| | TYLCAVNIGTSGTYKYIFGTGTRLKVLA | |
| 228 | atggatacctggctcgtgtgctgggccatcttcagcctgctgaaagccggactgaccgagcctg | TCR 49 Beta |
| | aagtgacccagacacctagccaccaagtgacacagatgggccaagaagtgatcctgcgctgcg | Var. + Constant |
| | tgcccatcagcaaccacctgtacttctactggtacagacagatcctgggccagaaagtggaattc | (nt) |
| | ctggtgtccttctacaacaacgagatcagcgagaagtccgagatcttcgacgaccagttcagcgt | |
| | ggaaagacccgacggcagcaacttcaccctgaagatcagaagcaccaagctcgaggacagc | |
| | gccatgtacttttgcgccagcacaccccggtacagctacgagcagtatttcggccctggcaccag | |
| | actgaccgtgaca | |
| 229 | EPEVTQTPSHQVTQMGQEVILRCVPISNHLYFYWYRQILGQK | TCR 49 Beta |
| | VEFLVSFYNNEISEKSEIFDDQFSVERPDGSNFTLKIRSTKLED | Var. + Constant |
| | SAMYFCASTPRYSYEQYFGPGTRLTVTEDLKNVFPPEVAVFE | (aa) |
| | PSEAEISHTQKATLVCLATGFYPDHVELSWWVNGKEVHSGV | |
| | CTDPQPLKEQPALNDSRYCLSSRLRVSATFWQNPRNHFRCQ | |
| | VQFYGLSENDEWTQDRAKPVTQIVSAEAWGRADCGFTSESY | |
| | QQGVLSATILYEILLGKATLYAVLVSALVLMAMVKRKDSRG | |
| 230 | EPEVTQTPSHQVTQMGQEVILRCVPISNHLYFYWYRQILGQK | TCR 49 Beta |
| | VEFLVSFYNNEISEKSEIFDDQFSVERPDGSNFTLKIRSTKLED | Var. (aa) |
| | SAMYFCASTPRYSYEQYFGPGTRLTVT | |
| 231 | DRGSQS | TCR 49 Alpha |
| | | CDR1 (aa) |
| 232 | IYSNGD | TCR 49 Alpha |
| | | CDR2 (aa) |
| 233 | AVNIGTSGTYKYI | TCR 49 Alpha |
| | | CDR3 (aa) |
| 234 | SNHLY | TCR 49 Beta |
| | | CDR1 (aa) |
| 235 | FYNNEI | TCR 49 Beta |
| | | CDR2 (aa) |
| 236 | ASTPRYSYEQY | TCR 49 Beta |
| | | CDR3 (aa) |
| 237 | atggccagcgctcctatctctatgctggccatgctgttcaccctgagcggactgagagcacagtc | TCR 53 Alpha |
| | tgtggcccagcctgaggaccaagtgaatgtggccgagggcaatcccctgaccgtgaagtgtacc | Var. + Constant |
| | tacagcgtgtccggcaatccctacctgttttggtacgtgcagtaccccaaccggggcctgcagtt | (nt) |
| | cctgctgaagtacatcaccggcgacaacctggtcaagggcagctatggattcgaggccgagttca | |
| | acaagagccagaccagcttccacctgaagaaacccagcgctctggtgtccgatagcgccctgta | |
| | tttctgcgccgtcagagattttggcagcggcacctacaagtacatcttcggcaccggcaccagac | |
| | tgaaggtgctggct | |
| 238 | AQSVAQPEDQVNVAEGNPLTVKCTYSVSGNPYLFWYVQYP | TCR 53 Alpha |
| | NRGLQFLLKYITGDNLVKGSYGFEAEFNKSQTSFHLKKPSAL | Var. + Constant |
| | VSDSALYFCAVRDFGSGTYKYIFGTGTRLKVLANIQNPDPAV | (aa) |
| | YQLRDSKSSDKSVCLFTDFDSQTNVSQSKDSDVYITDKCVLD | |
| | MRSMDFKSNSAVAWSNKSDFACANAFNNSIIPEDTFFPSPESS | |
| | CDVKLVEKSFETDTNLNFQNLSVIGFRILLLKVAGFNLLMTL | |
| | RLWSS | |
| 239 | AQSVAQPEDQVNVAEGNPLTVKCTYSVSGNPYLFWYVQYP | TCR 53 Alpha |
| | NRGLQFLLKYITGDNLVKGSYGFEAEFNKSQTSFHLKKPSAL | Var. (aa) |
| | VSDSALYFCAVRDFGSGTYKYIFGTGTRLKVLA | |
| 240 | atgggcacaagcctgctgtgttgggtcgtgctgggctttctgggcacagatcatacaggcgccg | TCR 53 Beta |
| | gtgtcagccagtctcctcggtacaaagtgaccaagcgcggacaggatgtggccctgagatgcg | Var. + Constant |
| | atcctatctctggccacgtgtccctgtactggtacagacaggctctcggacagggccccgagttc | (nt) |
| | ctgacctacttcaattacgaggcccagcaggacaagagcggcctgcctaacgatagattcagcg | |
| | ccgaaagacccgagggcagcatcagcacactgaccatccagagaaccgagcagagggacag | |
| | cgccatgtacagatgtgcctctagcggcagcggcaccagcggctacaatgagcagtttttcggc | |
| | cctggcaccagactgacagtgct | |
| 241 | GAGVSQSPRYKVTKRGQDVALRCDPISGHVSLYWYRQALG | TCR 53 Beta |
| | QGPEFLTYFNYEAQQDKSGLPNDRFSAERPEGSISTLTIQRTE | Var. + Constant |
| | QRDSAMYRCASSGSGTSGYNEQFFGPGTRLTVLEDLKNVFPP | (aa) |
| | EVAVFEPSEAEISHTQKATLVCLATGFYPDHVELSWWVNGK | |
| | EVHSGVCTDPQPLKEQPALNDSRYCLSSRLRVSATFWQNPR | |
| | NHFRCQVQFYGLSENDEWTQDRAKPVTQIVSAEAWGRADC | |
| | GFTSESYQQGVLSATILYEILLGKATLYAVLVSALVLMAMV | |
| | KRKDSRG | |
| 242 | GAGVSQSPRYKVTKRGQDVALRCDPISGHVSLYWYRQALG | TCR 53 Beta |
| | QGPEFLTYFNYEAQQDKSGLPNDRFSAERPEGSISTLTIQRTE | Var. (aa) |
| | QRDSAMYRCASSGSGTSGYNEQFFGPGTRLTVL | |
| 243 | VSGNPY | TCR 53 Alpha |
| | | CDR 1 (aa) |
| 244 | YITGDNLV | TCR 53 Alpha |
| | | CDR2 (aa) |
| 245 | AVRDFGSGTYKYI | TCR 53 Alpha |
| | | CDR3 (aa) |
| 246 | SGHVS | TCR 53 Beta |
| | | CDR1 (aa) |
| 247 | FNYEAQ | TCR 53 Beta |
| | | CDR2 (aa) |
| 248 | ASSGSGTSGYNEQF | TCR 53 Beta |
| | | CDR3 (aa) |
| 249 | DIVLTQSPPSLAMSLGKRATISCRASESVTILGSHLIHWYQQK | Anti-BCMA scFv |
| | PGQPPTLLIQLASNVQTGVPARFSGSGSRTDFTLTIDPVEEDD | |
| | VAVYYCLQSRTIPRTFGGGTKLEIKGSTSGSGKPGSGEGSTK | |
| | GQIQLVQSGPELKKPGETVKISCKASGYTFTDYSINWVKRAP | |
| | GKGLKWMGWINTETREPAYAYDFRGRFAFSLETSASTAYLQ | |
| | INNLKYEDTATYFCALDYSYAMDYWGQGTSVTVSS | |
| 250 | QSALTQPASVSASPGQSIAISCTGTSSDVGWYQQHPGKAPKL | Anti-BCMA scFv |
| | MIYEDSKRPSGVSNRFSGSKSGNTASLTISGLQAEDEADYYC | |
| | SSNTRSSTLVFGGGTKLTVLGSRGGGGSGGGGSGGGGSLEM | |
| | AEVQLVQSGAEMKKPGASLKLSCKASGYTFIDYYVYWMRQ | |
| | APGQGLESMGWINPNSGGTNYAQKFQGRVTMTRDTSISTAY | |
| | MELSRLRSDDTAMYYCARSQRDGYMDYWGQGTLVTVSS | |
| 251 | atggccaaggagaggggcctaataagccccagtgattttgcccagctgcaaaaatacatggaat | DGKα (nt) |
| | actccaccaaaaaggtcagtgatgtcctaaagctcttcgaggatggcgagatggctaaatatgtc | |
| | caaggagatgccattgggtacgagggattccagcaattcctgaaaatctatctcgaagtggataat | |
| | gttcccagacacctaagcctggcactgtttcaatcctttgagactggtcactgcttaaatgagaca | |
| | aatgtgacaaaagatgtggtgtgtctcaatgatgtttcctgctacttttcccttctggagggtggt | |
| | cggccagaagacaagttagaattcaccttcaagctgtacgacacggacagaaatgggatcctggac | |
| | agctcagaagtggacaaaattatcctacagatgatgcgagtggctgaatacctggattgggatgtg | |
| | tctgagctgaggccgattcttcaggagatgatgaaagagattgactatgatggcagtggctctgtc | |
| | tctcaagctgagtgggtccgggctggggccaccaccgtgccactgctagtgctgctgggtctgga | |
| | gatgactctgaaggacgacggacagcacatgtggaggcccaagaggttccccagaccagtcta | |
| | ctgcaatctgtgcgagtcaagcattggtcttggcaaacagggactgagctgtaacctctgtaagta | |
| | cactgttcacgaccagtgtgccatgaaagccctgccttgtgaagtcagcacctatgccaagtctc | |
| | ggaaggacattggtgtccaatcacatgtgtgggtgcgaggaggctgtgagtccgggcgctgcg | |
| | accgctgtcagaaaaagatccggatctaccacagtctgaccgggctgcattgtgtatggtgccac | |
| | ctagagatccacgatgactgcctgcaagcggtgggccatgagtgtgactgtgggctgctccggg | |
| | atcacatcctgcctccatcttccatctatcccagtgtcctggcctctggaccggatcgtaaaaata | |
| | gcaaaacaagccagaagaccatggatgatttaaatttgagcacctctgaggctctgcggattgacc | |
| | ctgttcctaacacccacccacttctcgtctttgtcaatcctaagagtggcgggaagcaggggcag | |
| | agggtgctctggaagttccagtatatattaaaccctcgacaggtgttcaacctcctaaaggatggt | |
| | cctgagatagggctccgattattcaaggatgttcctgatagccggattttggtgtgtggtggagac | |
| | ggcacagtaggctggattctagagaccattgacaaagctaacttgccagttttgcctcctgttgct | |
| | gtgttgcccctgggtactggaaatgatctggctcgatgcctaagatggggaggaggttatgaagga | |
| | cagaatctggcaaagatcctcaaggatttagagatgagtaaagtggtacatatggatcgatggtct | |
| | gtggaggtgatacctcaacaaactgaagaaaaaagtgacccagtcccctttcaaatcatcaataa | |
| | ctacttctctattggcgtggatgcctctattgctcatcgattccacatcatgcgagagaaatatcc | |
| | ggagaagttcaacagcagaatgaagaacaagctatggtacttcgaatttgccacatctgaatccat | |
| | cttctcaacatgcaaaaagctggaggagtctttgacagttgagatctgtgggaaaccgctggatct | |
| | gagcaacctgtccctagaaggcatcgcagtgctaaacatccctagcatgcatggtggctccaacct | |
| | ctggggtgataccaggagaccccatggggatatctatgggatcaaccaggccttaggtgctacag | |
| | ctaaagtcatcaccgaccctgatatcctgaaaacctgtgtaccagacctaagtgacaagagactg | |
| | gaagtggttgggctggagggtgcaattgagatgggccaaatctataccaagctcaagaatgctg | |
| | gacgtcggctggccaagtgctctgagatcaccttccacaccacaaaaacccttcccatgcaaatt | |
| | gacggagaaccctggatgcagacgccctgtacaatcaagatcacccacaagaaccagatgccc | |
| | atgctcatgggcccacccccccgctccaccaatttctttggcttcttgagcggatcctcggagac | |
| | agtgcggtttcagggacaccaccaccatcaccactga | |
| 252 | atggagccgcgggacggtagccccgaggcccggagcagcgactccgagtcggcttccgcct | DGKζ (nt) |
| | cgtccagcggctccgagcgcgacgccggtcccgagccggacaaggcgccgcggcgactca | |
| | acaagcggcgcttcccggggctgcggctcttcgggcacaggaaagccatcacgaagtcgggc | |
| | ctccagcacctggccccccctccgcccacccctggggccccgtgcagcgagtcagagcggca | |
| | gatccggagtacagtggactggagcgagtcagcgacatatggggagcacatctggttcgagac | |
| | caacgtgtccggggacttctgctacgttggggagcagtactgtgtagccaggatgctgcagaag | |
| | tcagtgtctcgaagaaagtgcgcagcctgcaagattgtggtgcacacgccctgcatcgagcagc | |
| | tggagaagataaatttccgctgtaagccgtccttccgtgaatcaggctccaggaatgtccgcgag | |
| | ccaacctttgtacggcaccactgggtacacagacgacgccaggacggcaagtgtcggcactgt | |
| | gggaagggattccagcagaagttcaccttccacagcaaggagattgtggccatcagctgctcgt | |
| | ggtgcaagcaggcataccacagcaaggtgtcctgcttcatgctgcagcagatcgaggagccgt | |
| | gctcgctgggggtccacgcagccgtggtcatcccgcccacctggatcctccgcgcccggagg | |
| | ccccagaatactctgaaagcaagcaagaagaagaagagggcatccttcaagaggaagtccag | |
| | caagaaagggcctgaggagggccgctggagacccttcatcatcaggcccaccccctccccgct | |
| | catgaagcccctgctggtgtttgtgaaccccaagagtgggggcaaccagggtgcaaagatcatc | |
| | cagtctttcctctggtatctcaatccccgacaagtcttcgacctgagccagggagggcccaagga | |
| | ggcgctggagatgtaccgcaaagtgcacaacctgcggatcctggcgtgcgggggcgacggca | |
| | cggtgggctggatcctctccaccctggaccagctacgcctgaagccgccaccccctgttgccat | |
| | cctgcccctgggtactggcaacgacttggcccgaaccctcaactggggtgggggctacacaga | |
| | tgagcctgtgtccaagatcctctcccacgtggaggaggggaacgtggtacagctggaccgctg | |
| | ggacctccacgctgagcccaaccccgaggcagggcctgaggaccgagatgaaggcgccacc | |
| | gaccggttgcccctggatgtcttcaacaactacttcagcctgggctttgacgcccacgtcaccctg | |
| | gagttccacgagtctcgagaggccaacccagagaaattcaacagccgctttcggaataagatgtt | |
| | ctacgccgggacagctttctctgacttcctgatgggcagctccaaggacctggccaagcacatcc | |
| | gagtggtgtgtgatggaatggacttgactcccaagatccaggacctgaaaccccagtgtgttgttt | |
| | tcctgaacatccccaggtactgtgcgggcaccatgccctggggccaccctggggagcaccacg | |
| | actttgagccccagcggcatgacgacggctacctcgaggtcattggcttcaccatgacgtcgttg | |
| | gccgcgctgcaggtgggcggacacggcgagcggctgacgcagtgtcgcgaggtggtgctca | |
| | ccacatccaaggccatcccggtgcaggtggatggcgagccctgcaagcttgcagcctcacgca | |
| | tccgcatcgccctgcgcaaccaggccaccatggtgcagaaggccaagcggcggagcgccgc | |
| | ccccctgcacagcgaccagcagccggtgccagagcagttgcgcatccaggtgagtcgcgtca | |
| | gcatgcacgactatgaggccctgcactacgacaaggagcagctcaaggaggcctctgtgccgc | |
| | tgggcactgtggtggtcccaggagacagtgacctagagctctgccgtgcccacattgagagact | |
| | ccagcaggagcccgatggtgctggagccaagtccccgacatgccagaaactgtcccccaagt | |
| | ggtgcttcctggacgccaccactgccagccgcttctacaggatcgaccgagcccaggagcacc | |
| | tcaactatgtgactgagatcgcacaggatgagatttatatcctggaccctgagctgctgggggcat | |
| | cggcccggcctgacctcccaacccccacttcccctctccccacctcaccctgctcacccacgcc | |
| | ccggtcactgcaaggggatgctgcaccccctcaaggtgaagagctgattgaggctgccaagag | |
| | gaacgacttctgtaagctccaggagctgcaccgagctgggggcgacctcatgcaccgagacga | |
| | gcagagtcgcacgctcctgcaccacgcagtcagcactggcagcaaggatgtggtccgctacct | |
| | gctggaccacgcccccccagagatccttgatgcggtggaggaaaacggggagacctgtttgca | |
| | ccaagcagcggccctgggccagcgcaccatctgccactacatcgtggaggccggggcctcgc | |
| | tcatgaagacagaccagcagggcgacactccccggcagcgggctgagaaggctcaggacac | |
| | cgagctggccgcctacctggagaaccggcagcactaccagatgatccagcgggaggaccag | |
| | gagacggctgtgggatcctcggagacagtgcggtttcagggacaccaccaccatcaccactga | |

1. A method of treatment, the method comprising administering to a subject having a cancer a combination therapy comprising:
(a) a T cell therapy comprising viable engineered T cells expressing a recombinant receptor directed against an antigen expressed by cells of the cancer, wherein the T cell therapy is administered on Day 1 of the combination therapy; and
(b) an inhibitor of DGKα and/or DGKζ, wherein the administration of the inhibitor is initiated on any of Days 1-8, inclusive, of the combination therapy, and the inhibitor is administered once daily for between or between about 21 and 42 consecutive days, inclusive.

2. The method of claim 1, wherein the administration of the inhibitor is initiated subsequent to the administration of the T cell therapy.

3. A method of treatment, the method comprising administering to a subject having a cancer an inhibitor of DGKα and/or DGKζ, wherein the inhibitor is administered as part of a combination therapy comprising the inhibitor and a T cell therapy comprising viable engineered T cells expressing a recombinant receptor directed against an antigen expressed by cells of the cancer, wherein:
prior to initiation of administration of the inhibitor, the subject has been previously administered the T cell therapy, wherein the T cell therapy is administered on Day 1 of the combination therapy;
the administration of the inhibitor is initiated on any of Days 1-8, inclusive, of the combination therapy; and
the inhibitor is administered once daily for between or between about 21 and 42 consecutive days, inclusive.

4. A method of treatment, the method comprising administering to a subject having a cancer a combination therapy comprising:
(a) a T cell therapy comprising viable engineered T cells expressing a recombinant receptor directed against an antigen expressed by cells of the cancer, wherein the T cell therapy is administered on Day 1 of the combination therapy; and
(b) an inhibitor of DGKα and/or DGKζ, wherein the administration of the inhibitor is initiated on any of Days 1-8, inclusive, of the combination therapy, and the inhibitor is administered continuously at least until the number of engineered T cells of the T cell therapy has peaked in the subject following administration of the T cell therapy.

5. The method of claim 4, wherein the administration of the inhibitor is initiated subsequent to the administration of the T cell therapy.

6. A method of treatment, the method comprising administering to a subject having a cancer an inhibitor of DGKα and/or DGKζ, wherein the inhibitor is administered as part of a combination therapy comprising the inhibitor and a T cell therapy comprising viable engineered T cells expressing a recombinant receptor directed against an antigen expressed by cells of the cancer, wherein:
prior to initiation of administration of the inhibitor, the subject has been previously administered the T cell therapy, wherein the T cell therapy is administered on Day 1 of the combination therapy;
the administration of the inhibitor is initiated on any of Days 1-8, inclusive, of the combination therapy; and
the inhibitor is administered continuously at least until the number of engineered T cells of the T cell therapy has peaked in the subject following administration of the T cell therapy.

7. The method of claim 4, wherein the administration of the inhibitor is initiated on Day 1 of the combination therapy prior to the administration of the T cell therapy.

8. A method of treatment, the method comprising administering to a subject having a cancer a T cell therapy comprising viable engineered T cells expressing a recombinant receptor directed against an antigen expressed by cells of the cancer, wherein the T cell therapy is administered as part of a combination therapy comprising the T cell therapy and an inhibitor of DGKα and/or DGKζ, wherein:
the T cell therapy is administered on Day 1 of the combination therapy;
the administration of the inhibitor is initiated on Day 1 of the combination therapy prior to the administration of the T cell therapy; and
the inhibitor is administered continuously at least until the number of engineered T cells of the T cell therapy has peaked in the subject following administration of the T cell therapy.

9. The method of any one of claims 4-8, wherein the inhibitor is administered daily, every two days, or every three days.

10. The method of any one of claims 4-9, wherein the inhibitor is administered daily.

11. The method of any one of claims 4-10, wherein the inhibitor is administered once on each of the days that the inhibitor is administered.

12. The method of any one of claims 4-11, wherein the inhibitor is administered until the number of engineered T cells of the T cell therapy has peaked in the subject following administration of the T cell therapy.

13. The method of any one of claims 4-12, wherein the inhibitor is administered over a period of time between or between about 21 and 42 days, inclusive.

14. A method of treatment, the method comprising administering to a subject having a cancer a combination therapy comprising:
(a) a T cell therapy comprising viable engineered T cells expressing a recombinant receptor directed against an antigen expressed by cells of the cancer, wherein the T cell therapy is administered on Day 1 of the combination therapy; and
(b) an inhibitor of DGKα and/or DGKζ, wherein the administration of the inhibitor is initiated on any of Days 1-8, inclusive, of the combination therapy, and the inhibitor is administered in a therapeutically effective dose for a period between or between about 21 and 42 days.

15. The method of claim 14, wherein the administration of the inhibitor is initiated subsequent to the administration of the T cell therapy.

16. A method of treatment, the method comprising administering to a subject having a cancer an inhibitor of DGKα and/or DGKζ, wherein the inhibitor is administered as part of a combination therapy comprising the inhibitor and a T cell therapy comprising viable engineered T cells expressing a recombinant receptor directed against an antigen expressed by cells of the cancer, wherein:
prior to initiation of administration of the inhibitor, the subject has been previously administered the T cell therapy, wherein the T cell therapy is administered on Day 1 of the combination therapy;
the administration of the inhibitor is initiated on any of Days 1-8, inclusive, of the combination therapy; and
the inhibitor is administered in a therapeutically effective dose for a period between or between about 21 and 42 days.

17. The method of claim 14, wherein the administration of the inhibitor is initiated on Day 1 of the combination therapy prior to the administration of the T cell therapy.

18. A method of treatment, the method comprising administering to a subject having a cancer a T cell therapy comprising viable engineered T cells expressing a recombinant receptor directed against an antigen expressed by cells of the cancer, wherein the T cell therapy is administered as part of a combination therapy comprising the T cell therapy and an inhibitor of DGKα and/or DGKζ, wherein:
the T cell therapy is administered on Day 1 of the combination therapy;
the administration of the inhibitor is initiated on Day 1 of the combination therapy prior to the administration of the T cell therapy; and
the inhibitor is administered in a therapeutically effective dose for a period between or between about 21 and 42 days.

19. The method of any one of claims 14-18, wherein the therapeutically effective dose provides a dose of the inhibitor that is continuously therapeutically effective for the period between or between about 21 and 42 days.

20. The method of any one of claims 14-19, wherein the inhibitor is administered every 2 days or 3 days.

21. A method of treatment, the method comprising administering to a subject having a cancer a combination therapy comprising:
(a) a T cell therapy comprising viable engineered T cells expressing a recombinant receptor directed against an antigen expressed by cells of the cancer, wherein the T cell therapy is administered on Day 1 of the combination therapy; and
(b) an inhibitor of DGKα and/or DGKζ, wherein the administration of the inhibitor is initiated on any of Days 1-8, inclusive, of the combination therapy, and the inhibitor is administered in a plurality of cycles each comprising an administration period in which the inhibitor is administered followed by a rest period during which the inhibitor is not administered.

22. The method of claim 21, wherein the administration of the inhibitor is initiated subsequent to the administration of the T cell therapy.

23. A method of treatment, the method comprising administering to a subject having a cancer an inhibitor of DGKα and/or DGKζ, wherein the inhibitor is administered as part of a combination therapy comprising the inhibitor and a T cell therapy comprising viable engineered T cells expressing a recombinant receptor directed against an antigen expressed by cells of the cancer, wherein:
prior to initiation of administration of the inhibitor, the subject has been previously administered the T cell therapy, wherein the T cell therapy is administered on Day 1 of the combination therapy;
the administration of the inhibitor is initiated on any of Days 1-8, inclusive, of the combination therapy; and
the inhibitor is administered in a plurality of cycles each comprising an administration period in which the inhibitor is administered followed by a rest period during which the inhibitor is not administered.

24. The method of any one of claims 21-23, wherein during the administration period, the inhibitor is administered daily, every two days, or every three days.

25. The method of any one of claims 21-24, wherein during the administration period, the inhibitor is administered daily.

26. The method of any one of claims 21-25, wherein during the administration period, the inhibitor is administered once on each of the days that the inhibitor is administered.

27. The method of any one of claims 21-26, wherein over the plurality of cycles, the inhibitor is administered until the number of engineered T cells of the T cell therapy has peaked in the subject following administration of the T cell therapy.

28. The method of any one of claims 21-27, wherein over the plurality of cycles, the inhibitor is administered over a period of time between or between about 21 and 42 days, inclusive.

29. The method of any one of claims 1-28, wherein the administration of the inhibitor is initiated on any of Days 1-6, inclusive, of the combination therapy, on any of Days 1-4, inclusive, of the combination therapy, on Day 1 or Day 2 of the combination therapy, and/or on Day 1 of the combination therapy.

30. The method of claim 1 or claim 29, wherein the administration of the inhibitor is initiated on Day 1 of the combination therapy prior to the administration of the T cell therapy.

31. A method of treatment, the method comprising administering to a subject having a cancer a T cell therapy comprising viable engineered T cells expressing a recombinant receptor directed against an antigen expressed by cells of the cancer, wherein the T cell therapy is administered as part of a combination therapy comprising the T cell therapy and an inhibitor of DGKα and/or DGKζ, wherein:
the T cell therapy is administered on Day 1 of the combination therapy;
the administration of the inhibitor is initiated on Day 1 of the combination therapy prior to the administration of the T cell therapy; and
the inhibitor is administered once daily for between or between about 21 and 42 consecutive days, inclusive.

32. The method of any one of claims 1-31, wherein the inhibitor, optionally over the plurality of cycles, is administered over a period of time between or between about 21 and 35 days, inclusive, a period of time between or between about 21 and 28 days, inclusive; a period of time between or between about 28 and 42 days, inclusive; a period of time between or between about 28 and 35 days, inclusive; a period of time that is or is about 28 days; and/or a period of time that is or is about 30 days.

33. A method of treatment, the method comprising administering to a subject having a cancer a combination therapy comprising:
(a) a T cell therapy comprising viable engineered T cells expressing a recombinant receptor directed against an antigen expressed by cells of the cancer, wherein the T cell therapy is administered on Day 1 of the combination therapy; and
(b) an inhibitor of DGKα and/or DGKζ, wherein the administration of the inhibitor is initiated on Day 1 of the combination therapy, and the inhibitor is administered once daily on Days 1-28, inclusive, of the combination therapy.

34. The method of claim 33, wherein the administration of the inhibitor is initiated subsequent to the administration of the T cell therapy.

35. A method of treatment, the method comprising administering to a subject having a cancer an inhibitor of DGKα and/or DGKζ, wherein the inhibitor is administered as part of a combination therapy comprising the inhibitor and a T cell therapy comprising viable engineered T cells expressing a recombinant receptor directed against an antigen expressed by cells of the cancer, wherein:
prior to initiation of administration of the inhibitor, the subject has been previously administered the T cell therapy, wherein the T cell therapy is administered on Day 1 of the combination therapy;
the administration of the inhibitor is initiated on Day 1 of the combination therapy; and
the inhibitor is administered once daily on Days 1-28, inclusive, of the combination therapy.

36. The method of claim 33, wherein the administration of the inhibitor is initiated prior to the administration of the T cell therapy.

37. A method of treatment, the method comprising administering to a subject having a cancer a T cell therapy comprising viable engineered T cells expressing a recombinant receptor directed against an antigen expressed by cells of the cancer, wherein the T cell therapy is administered as part of a combination therapy comprising the T cell therapy and an inhibitor of DGKα and/or DGKζ, wherein:
the T cell therapy is administered on Day 1 of the combination therapy;
the administration of the inhibitor is initiated on Day 1 of the combination therapy prior to the administration of the T cell therapy; and
the inhibitor is administered once daily on Days 1-28, inclusive, of the combination therapy.

38. The method of any one of claims 33-37, wherein the inhibitor is administered once daily on Days 1-30, inclusive, of the combination therapy.

39. The method of any one of claims 1-38, wherein the administration of the inhibitor is initiated within or within about 12 hours of the administration of the T cell therapy, within or within about 6 hours of the administration of the T cell therapy, or within or within about 4 hours of the administration of the T cell therapy.

40. The method of any one of claims 1-39, wherein the administration of the inhibitor is initiated within or within about 2 hours of the administration of the T cell therapy.

41. The method of any one of claims 1-40, wherein the administration of the inhibitor is initiated within or within about 1 hour of the administration of the T cell therapy.

42. The method of any one of claims 1, 4, 9-14, 19-24, 27-32, and 37-41, wherein the administration of the inhibitor is initiated on Day 1 of the combination therapy concurrently with the administration of the T cell therapy.

43. The method of any one of claims 1, 4, 9-14, 19-24, 27-32, and 37-42, wherein the administration of the inhibitor is initiated on Day 1 of the combination therapy during the administration of the T cell therapy.

44. The method of any one of claims 1-43, wherein prior to the administration of the T cell therapy, the subject has been preconditioned with a lymphodepleting therapy comprising administration of fludarabine and/or cyclophosphamide.

45. The method of claim 44, wherein the method further comprises administering the lymphodepleting therapy to the subject.

46. The method of claim 44 or claim 45, wherein the lymphodepleting therapy comprises administration of:
cyclophosphamide at between or between about 200 and 400 mg/m², inclusive, daily for between or between about 2 and 4 days, inclusive; and/or
fludarabine at between or between about 20 and 40 mg/m², inclusive, daily for between or between about 2 and 4 days, inclusive.

47. The method of any one of claims 44-46, wherein the lymphodepleting therapy comprises administration of cyclophosphamide at or at about 300 mg/m² and fludarabine at or at about 30 mg/m² each daily for or for about 3 days.

48. The method of any one of claims 44-47, wherein the T cell therapy is administered between or between about 2 and 7 days, inclusive, after the administration of the lymphodepleting therapy.

49. The method of any one of claims 1-48, wherein the inhibitor is an inhibitor of DGKα and not a significant inhibitor of DGKζ.

50. The method of any one of claims 1-48, wherein the inhibitor is an inhibitor of DGK and not a significant inhibitor of DGKα.

51. The method of any one of claims 1-48, wherein the inhibitor is an inhibitor of DGKα and DGKζ.

52. The method of any one of claims 1-51, wherein the inhibitor is not a significant inhibitor of other DGKs.

53. The method of any one of claims 1-52, wherein the inhibitor is a compound of Formula (I): 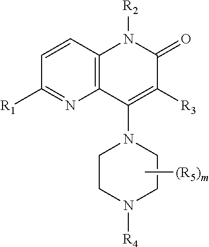
or a pharmaceutically acceptable salt thereof, wherein:
R₁ is H, F, Cl, Br, —CN, C₁₋₃ alkyl substituted with zero to 4 R₁ₐ, C₃₋₄ cycloalkyl substituted with zero to 4 R₁ₐ, C₁₋₃ alkoxy substituted with zero to 4 R₁ₐ, —NRₐRₐ, —S(O)ₙRₑ, or —P(O)RₑRₑ;
each R₁ₐ is independently F, Cl, —CN, —OH, —OCH₃, or —NRₐRₐ;
each Rₐ is independently H or C₁₋₃ alkyl;
each Rₑ is independently C₃₋₄ cycloalkyl or C₁₋₃ alkyl substituted with zero to 4 R₁ₐ;
R₂ is H, C₁₋₃ alkyl substituted with zero to 4 R₂ₐ, or C₃₋₄ cycloalkyl substituted with zero to 4 R₂ₐ;
each R₂ₐ is independently F, Cl, —CN, —OH, —O(C₁₋₂ alkyl), C₃₋₄ cycloalkyl, C₃₋₄ alkenyl, or C₃₋₄ alkynyl;
R₃ is H, F, Cl, Br, —CN, C₁₋₃ alkyl, C₁₋₂ fluoroalkyl, C₃₋₄ cycloalkyl, C₃₋₄ fluorocycloalkyl, or —NO₂;
R₄ is —CH₂R₄ₐ, —CH₂CH₂R₄ₐ, —CH₂CHR₄ₐR_{4d}, —CHR₄ₐR_{4b}, or —CR₄ₐR_{4b}R_{4c};
R₄ₐ and R_{4b} are independently:
(i) C₁₋₆ alkyl substituted with zero to 4 substituents independently selected from F, Cl, —CN, —OH, —OCH₃, —SCH₃, C₁₋₃ fluoroalkoxy, —NRₐRₐ, —S(O)₂Rₑ, or —NRₐS(O)₂Rₑ;
(ii) C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, C₁₋₄ hydroxyalkyl, —(CH₂)₁₋₂O(C₁₋₃ alkyl), C₁₋₄ alkoxy, —O(C₁₋₄ hydroxyalkyl), —O(CH)₁₋₃O(C₁₋₃ alkyl), C₁₋₃ fluoroalkoxy, —O(CH)₁₋₃NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₃ alkyl)₂, —S(O)₂(C₁₋₃ alkyl), —O(CH₂)₁₋₂(C₃₋₆ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or
(iii) C₁₋₄ alkyl substituted with one cyclic group selected from C₃₋₆ cycloalkyl, heterocyclyl, aryl, and heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl;
or R₄ₐ and R_{4b} together with the carbon atom to which they are attached form a C₃₋₆ cycloalkyl or a 3- to 6-membered heterocyclyl, each substituted with zero to 3 R_{f};
each R_{f} is independently F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, and —NR_{c}R_{c};
R_{4c} is C₁₋₆ alkyl or C₃₋₆ cycloalkyl, each substituted with zero to 4 substituents independently selected from F, Cl, —OH, C₁₋₂ alkoxy, C₁₋₂ fluoroalkoxy, and —CN;
R_{4d} is —OCH₃;
each R_{c} is independently H or C₁₋₂ alkyl;
R_{d} is phenyl substituted with zero to 1 substituent selected from F, Cl, —CN, —CH₃, and —OCH₃;
each R₅ is independently —CN, C₁₋₆ alkyl substituted with zero to 4 R_{g}, C₂₋₄ alkenyl substituted with zero to 4 R_{g}, C₂₋₄ alkynyl substituted with zero to 4 R_{g}, C₃₋₄ cycloalkyl substituted with zero to 4 R_{g}, phenyl substituted with zero to 4 R_{g}, oxadiazolyl substituted with zero to 3 R_{g}, pyridinyl substituted with zero to 4 R_{g}, —(CH₂)₁₋₂(heterocyclyl substituted with zero to 4 R_{g}), —(CH₂)₁₋₂NR_{c}C(O)(C₁₋₄ alkyl), —(CH₂)₁₋₂NRₐC(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each R_{g} is independently F, Cl, —CN, —OH, C₁₋₃ alkoxy, C₁₋₃ fluoroalkoxy, —O(CH₂)₁₋₂O(C₁₋₂ alkyl), or —NR_{c}R_{c};
m is zero, 1, 2, or 3; and
n is zero, 1, or 2.

54. The method of claim 53, wherein the inhibitor is a compound of Formula (I) or a pharmaceutically acceptable salt thereof, wherein:
R₁ is H, F, Cl, Br, —CN, C₁₋₃ alkyl substituted with zero to 4 R₁ₐ, cyclopropyl substituted with zero to 3 R₁ₐ, C₁₋₃ alkoxy substituted with zero to 3 R₁ₐ, —NRₐRₐ, —S(O)ₙCH₃, or —P(O)(CH₃)₂;
each R₁ₐ is independently F, Cl, or —CN;
each Rₐ is independently H or C₁₋₃ alkyl;
R₂ is H or C₁₋₂ alkyl substituted with zero to 2 R₂ₐ;
each R₂ₐ is independently F, Cl, —CN, —OH, —O(C₁₋₂ alkyl), cyclopropyl, C₃₋₄ alkenyl, or C₃₋₄ alkynyl;
R₃ is H, F, Cl, Br, —CN, C₁₋₂ alkyl, —CF₃, cyclopropyl, or —NO₂;
R₄ₐ and R_{4b} are independently:
(i) C₁₋₄ alkyl substituted with zero to 4 substituents independently selected from F, Cl, —CN, —OH, —OCH₃, —SCH₃, C₁₋₃ fluoroalkoxy, and —NRₐRₐ;
(ii) C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, —CH₂OH, —(CH₂)₁₋₂O(C₁₋₂ alkyl), C₁₋₄ alkoxy, —O(C₁₋₄ hydroxyalkyl), —O(CH)₁₋₂O(C₁₋₂ alkyl), C₁₋₃ fluoroalkoxy, —O(CH)₁₋₂NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —O(CH₂)₁₋₂(C₃₋₄ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or
(iii) C₁₋₃ alkyl substituted with one cyclic group selected from C₃₋₆ cycloalkyl, heterocyclyl, phenyl, and heteroaryl, said cyclic group substituted with zero to 3 substituents independently selected from F, Cl, Br, —OH, —CN, C₁₋₃ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₄ cycloalkyl;
or R₄ₐ and R_{4b} together with the carbon atom to which they are attached, form a C₃₋₆ cycloalkyl or a 3- to 6-membered heterocyclyl, each substituted with zero to 3 R_{f};
each R_{f} is independently F, Cl, Br, —OH, —CN, C₁₋₄ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₄ alkyl, C₁₋₂ fluoroalkyl, C₁₋₃ alkoxy, C₁₋₂ fluoroalkoxy, and —NR_{c}R_{c};
R_{4c} is C₁₋₄ alkyl or C₃₋₆ cycloalkyl, each substituted with zero to 4 substituents independently selected from F, Cl, —OH, C₁₋₂ alkoxy, C₁₋₂ fluoroalkoxy, and —CN;
and each R₅ is independently —CN, C₁₋₅ alkyl substituted with zero to 4 R_{g}, C₂₋₃ alkenyl substituted with zero to 4 R_{g}, C₂₋₃ alkynyl substituted with zero to 4 R_{g}, C₃₋₄ cycloalkyl substituted with zero to 4 R_{g}, phenyl substituted with zero to 3 R_{g}, oxadiazolyl substituted with zero to 3 R_{g}, pyridinyl substituted with zero to 3 R_{g}, —(CH₂)₁₋₂(heterocyclyl substituted with zero to 4 R_{g}), —(CH₂)₁₋₂NR_{c}C(O)(C₁₋₄ alkyl), —(CH₂)₁₋₂NRₐC(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl).

55. The method of claim 54, wherein the inhibitor is a compound of Formula (I) or a pharmaceutically acceptable salt thereof having the structure: 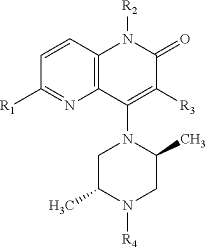
wherein:
R₁ is —CN;
R₂ is —CH₃;
R₃ is H, F, or —CN;
R₄ is:

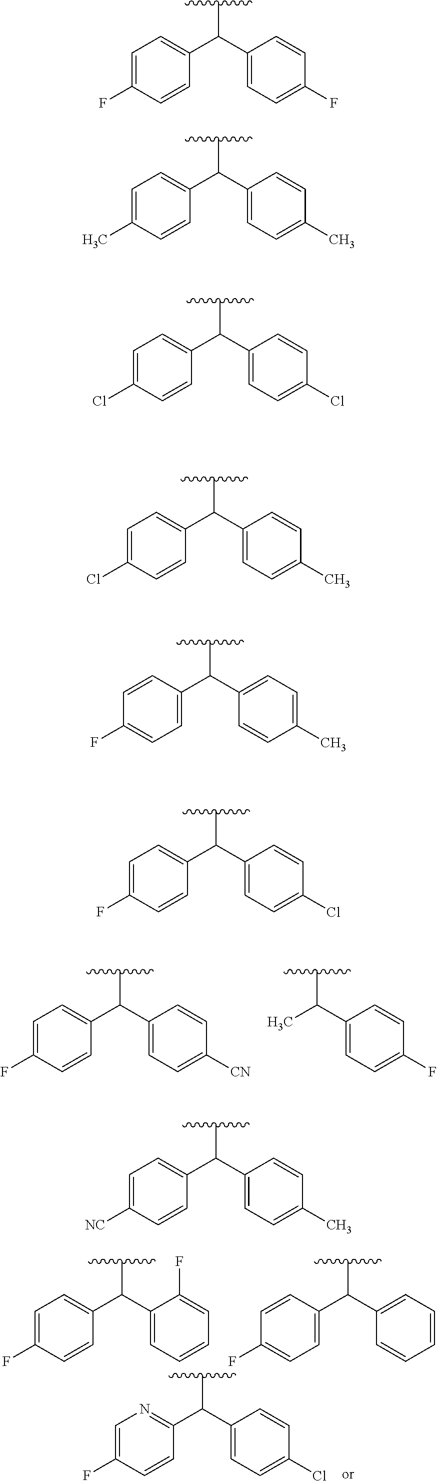

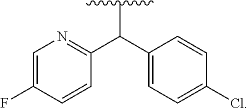 [SMILES: *C(c1ccc(Cl)cc1)c1ccc(F)cn1]

56. The method of claim 53, wherein the inhibitor is a compound of Formula (I) or a pharmaceutically acceptable salt thereof having the structure:

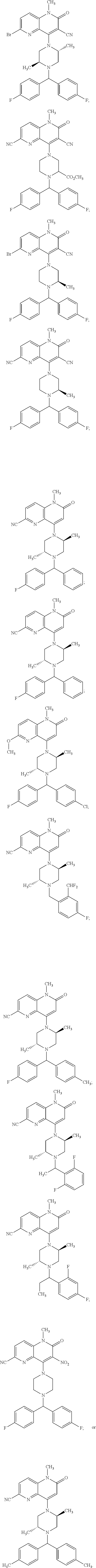

57. The method of any one of claims 1-52, wherein the inhibitor is a compound of Formula (II): 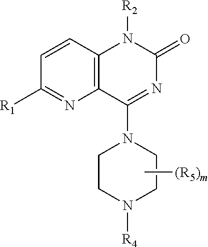
or a salt thereof, wherein:
R₁ is H, F, Cl, Br, —CN, —OH, C₁₋₃ alkyl substituted with zero to 4 R₁ₐ, C₃₋₄ cycloalkyl substituted with zero to 4 R₁ₐ, C₁₋₃ alkoxy substituted with zero to 4 R₁ₐ, —NRₐRₐ, —S(O)ₙRₑ, or —P(O)RₑRₑ;
each R₁ₐ is independently F, Cl, —CN, —OH, —OCH₃, or —NRₐRₐ;
each Rₐ is independently H or C₁₋₃ alkyl;
each Rₑ is independently C₃₋₄ cycloalkyl or C₁₋₃ alkyl substituted with zero to 4 R₁ₐ;
R₂ is H, C₁₋₃ alkyl substituted with zero to 4 R₂ₐ, or C₃₋₄ cycloalkyl substituted with zero to 4 R₂ₐ;
each R₂ₐ is independently F, Cl, —CN, —OH, —O(C₁₋₂ alkyl), C₃₋₄ cycloalkyl, C₃₋₄ alkenyl, or C₃₋₄ alkynyl;
R₄ is —CH₂R₄ₐ, —CH₂CH₂R₄ₐ, —CH₂CHR₄ₐR_{4d}, —CHR₄ₐR_{4b}, or —CR₄ₐR_{4b}R_{4c};
R₄ₐ and R_{4b} are independently:
(i) —CN or C₁₋₆ alkyl substituted with zero to 4 substituents independently selected from F, Cl, —CN, —OH, —OCH₃, —SCH₃, C₁₋₃ fluoroalkoxy, —NRₐRₐ, —S(O)₂Rₑ, or —NRₐS(O)₂Rₑ;
(ii) C₃₋₆ cycloalkyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₄ hydroxyalkyl, —(CH₂)₁₋₂O(C₁₋₃ alkyl), C₁₋₄ alkoxy, C₁₋₃ fluoroalkoxy, C₁₋₃ cyanoalkoxy, —O(C₁₋₄ hydroxyalkyl), —O(CRₓRₓ)₁₋₃O(C₁₋₃ alkyl), C₁₋₃ fluoroalkoxy, —O(CH₂)₁₋₃NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —CH₂NRₐRₐ, —NRₐS(O)₂(C₁₋₃ alkyl), —NRₐC(O)(C₁₋₃ alkyl), —(CRₓRₓ)₀₋₂NRₐC(O)O(C₁₋₄ alkyl), —P(O)(C₁₋₃ alkyl)₂, —S(O)₂(C₁₋₃ alkyl), —(CRₓRₓ)₁₋₂(C₃₋₄ cycloalkyl), —(CRₓRₓ)₁₋₂(morpholinyl), —(CRₓRₓ)₁₋₂(difluoromorpholinyl), —(CRₓRₓ)₁₋₂(dimethylmorpholinyl), —(CRₓRₓ)₁₋₂(oxaazabicyclo[2.2.1]heptanyl), (CRₓRₓ)₁₋₂(oxaazaspiro[3.3]heptanyl), —(CRₓRₓ)₁₋₂(methylpiperazinonyl), —(CRₓRₓ)₁₋₂(acetylpiperazinyl), —(CRₓRₓ)₁₋₂(piperidinyl), —(CRₓRₓ)₁₋₂(difluoropiperidinyl), —(CRₓRₓ)₁₋₂(methoxypiperidinyl), —(CRₓRₓ)₁₋₂(hydroxypiperidinyl), —O(CRₓRₓ)₀₋₂(C₃₋₆ cycloalkyl), —O(CRₓRₓ)₀₋₂(methylcyclopropyl), —O(CRₓRₓ)₀₋₂((ethoxycarbonyl)cyclopropyl), —O(CRₓRₓ)₀₋₂(oxetanyl), —O(CRₓRₓ)₀₋₂(methylazetidinyl), —O(CRₓRₓ)₀₋₂(tetrahydropyranyl), —O(CRₓRₓ)₁₋₂(morpholinyl), —O(CRₓRₓ)₀₋₂(thiazolyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, dioxolanyl, pyrrolidinonyl, and R_{d}; or
(iii) C₁₋₄ alkyl substituted with one cyclic group selected from C₃₋₆ cycloalkyl, 4- to 10-membered heterocyclyl, mono- or bicyclic aryl, or 5- to 10-membered heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl;
or R₄ₐ and R_{4b} together with the carbon atom to which they are attached form a C₃₋₆ cycloalkyl or a 3- to 6-membered heterocyclyl, each substituted with zero to 3 R_{f};
each R_{f} is independently F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, and —NR_{c}R_{c};
R_{4c} is C₁₋₆ alkyl or C₃₋₆ cycloalkyl, each substituted with zero to 4 substituents independently selected from F, Cl, —OH, C₁₋₂ alkoxy, C₁₋₂ fluoroalkoxy, and —CN;
R₄ₐ is —OCH₃;
each R_{c} is independently H or C₁₋₂ alkyl;
R_{d} is phenyl substituted with zero to 1 substituent selected from F, Cl, —CN, —CH₃, and —OCH₃;
each R₅ is independently —CN, C₁₋₆ alkyl substituted with zero to 4 R_{g}, C₂₋₄ alkenyl substituted with zero to 4 R_{g}, C₂₋₄ alkynyl substituted with zero to 4 R_{g}, C₃₋₄ cycloalkyl substituted with zero to 4 R_{g}, phenyl substituted with zero to 4 R_{g}, oxadiazolyl substituted with zero to 3 R_{g}, pyridinyl substituted with zero to 4 R_{g}, —(CH₂)₁₋₂(4- to 10-membered heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each R_{g} is independently F, Cl, —CN, —OH, C₁₋₃ alkoxy, C₁₋₃ fluoroalkoxy, —O(CH₂)₁₋₂O(C₁₋₂ alkyl), or —NR_{c}R_{c};
m is zero, 1, 2, or 3; and
n is zero, 1, or 2.

58. The method of claim 57, wherein the inhibitor is a compound of Formula (II) or a pharmaceutically acceptable salt thereof, wherein:
R₁ is H, F, Cl, Br, —CN, —OH, C₁₋₃ alkyl substituted with zero to 4 R₁ₐ, cyclopropyl substituted with zero to 3 R₁ₐ, C₁₋₃ alkoxy substituted with zero to 3 R₁ₐ, —NRₐRₐ, —S(O)ₙCH₃, or —P(O)(CH₃)₂;
R₂ is H or C₁₋₂ alkyl substituted with zero to 2 R₂ₐ;
each R₂ₐ is independently F, Cl, —CN, —OH, —O(C₁₋₂ alkyl), cyclopropyl, C₃₋₄ alkenyl, or C₃₋₄ alkynyl;
R₄ₐ and R_{4b} are independently:
(i) —CN or C₁₋₄ alkyl substituted with zero to 4 substituents independently selected from F, Cl, —CN, —OH, —OCH₃, —SCH₃, C₁₋₃ fluoroalkoxy, and —NRₐRₐ;
(ii) C₃₋₆ cycloalkyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₂ hydroxyalkyl, —CH₂NRₐRₐ, —(CH₂)₁₋₂O(C₁₋₂ alkyl), —(CH₂)₁₋₂NRₓC(O)O(C₁₋₂ alkyl), C₁₋₄ alkoxy, —O(C₁₋₄ hydroxyalkyl), —O(CRₓRₓ)₁₋₂O(C₁₋₂ alkyl), C₁₋₃ fluoroalkoxy, C₁₋₃ cyanoalkoxy, —O(CH₂)₁₋₂NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —(CH₂)₁₋₂(C₃₋₄ cycloalkyl), —CRₓRₓ(morpholinyl), —CRₓRₓ(difluoromorpholinyl), —CRₓRₓ(dimethylmorpholinyl), —CRₓRₓ(oxaazabicyclo[2.2.1]heptanyl), —CRₓRₓ(oxaazaspiro[3.3]heptanyl), —CRₓRₓ(methylpiperazinonyl), —CRₓRₓ(acetylpiperazinyl), —CRₓRₓ(piperidinyl), —CRₓRₓ(difluoropiperidinyl), —CRₓRₓ(methoxypiperidinyl), —CRₓRₓ(hydroxypiperidinyl), —O(CH₂)₀₋₂(C₃₋₄ cycloalkyl), —O(CH₂)₀₋₂(methylcyclopropyl), —O(CH₂)₀₋₂((ethoxycarbonyl)cyclopropyl), —O(CH₂)₀₋₂(oxetanyl), —O(CH₂)₀₋₂(methylazetidinyl), —O(CH₂)₁₋₂(morpholinyl), —O(CH₂)₀₋₂(tetrahydropyranyl), —O(CH₂)₀₋₂(thiazolyl), cyclopropyl, cyanocyclopropyl, methylazetidinyl, acetylazetidinyl, (tert-butoxycarbonyl)azetidinyl, dioxolanyl, pyrrolidinonyl, triazolyl, tetrahydropyranyl, morpholinyl, thiophenyl, methylpiperidinyl, and R_{d}; or
(iii) C₁₋₃ alkyl substituted with one cyclic group selected from C₃₋₆ cycloalkyl, 4- to 10-membered heterocyclyl, mono- or bicyclic aryl, or 5- to 10-membered heteroaryl, said cyclic group substituted with zero to 3 substituents independently selected from F, Cl, Br, —OH, —CN, C₁₋₃ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₄ cycloalkyl;
or R₄ₐ and R_{4b} together with the carbon atom to which they are attached, form a C₃₋₆ cycloalkyl or a 3- to 6-membered heterocyclyl, each substituted with zero to 3 R_{f};
each R_{f} is independently F, Cl, Br, —OH, —CN, C₁₋₄ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₄ alkyl, C₁₋₂ fluoroalkyl, C₁₋₃ alkoxy, C₁₋₂ fluoroalkoxy, and —NR_{c}R_{c};
R_{4c} is C₁₋₄ alkyl or C₃₋₆ cycloalkyl, each substituted with zero to 4 substituents independently selected from F, Cl, —OH, C₁₋₂ alkoxy, C₁₋₂ fluoroalkoxy, and —CN;
each R₅ is independently —CN, C₁₋₅ alkyl substituted with zero to 4 R_{g}, C₂₋₃ alkenyl substituted with zero to 4 R_{g}, C₂₋₃ alkynyl substituted with zero to 4 R_{g}, C₃₋₄ cycloalkyl substituted with zero to 4 R_{g}, phenyl substituted with zero to 3 R_{g}, oxadiazolyl substituted with zero to 3 R_{g}, pyridinyl substituted with zero to 3 R_{g}, —(CH₂)₁₋₂(4- to 10-membered heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each Rₓ is independently H or —CH₃; and
m is 1, 2, or 3.

59. The method of claim 58, wherein the inhibitor is a compound of Formula (II) or a pharmaceutically acceptable salt thereof having the structure: 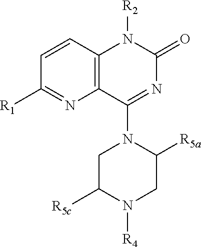
R₁ is —CN;
R₂ is —CH₃;
R₅ₐ is —CH₃ or —CH₂CH₃; and
R_{5c} is —CH₃, —CH₂CH₃, or —CH₂CH₂CH₃.

60. The method of claim 57, wherein the inhibitor is a compound of Formula (II) or a pharmaceutically acceptable salt thereof having the structure:

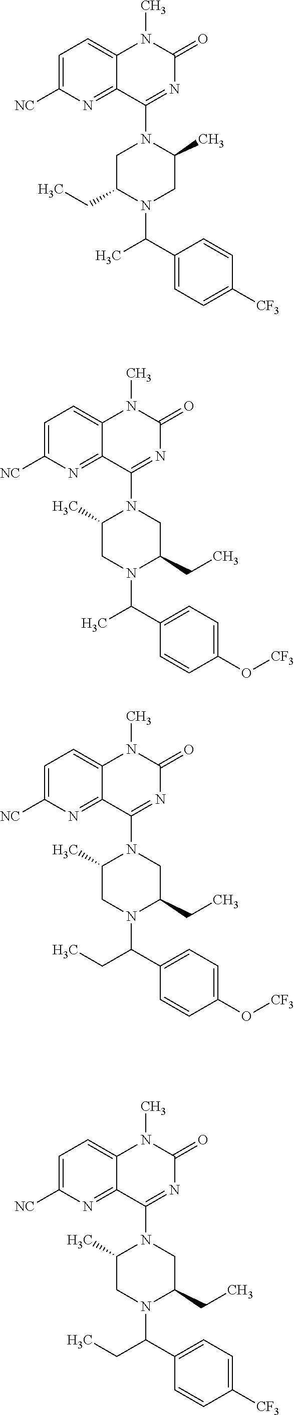 [SMILES: CC[C@@H]1CN(c2nc(=O)n(C)c3ccc(C#N)nc23)[C@@H](C)CN1C(C)c1ccc(C(F)(F)F)cc1 ; CC[C@@H]1CN(c2nc(=O)n(C)c3ccc(C#N)nc23)[C@@H](C)CN1C(C)c1ccc(OC(F)(F)F)cc1 ; CCC(c1ccc(OC(F)(F)F)cc1)N1C[C@H](C)N(c2nc(=O)n(C)c3ccc(C#N)nc23)C[C@H]1CC ; CCC(c1ccc(C(F)(F)F)cc1)N1C[C@H](C)N(c2nc(=O)n(C)c3ccc(C#N)nc23)C[C@H]1CC]

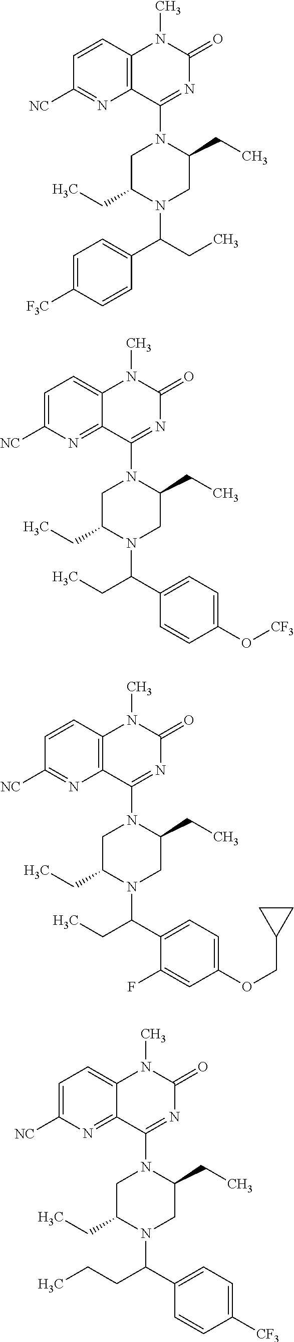 [SMILES: CCC(c1ccc(C(F)(F)F)cc1)N1C[C@H](CC)N(c2nc(=O)n(C)c3ccc(C#N)nc23)C[C@H]1CC ; CCC(c1ccc(OC(F)(F)F)cc1)N1C[C@H](CC)N(c2nc(=O)n(C)c3ccc(C#N)nc23)C[C@H]1CC ; CCC(c1ccc(OCC2CC2)cc1F)N1C[C@H](CC)N(c2nc(=O)n(C)c3ccc(C#N)nc23)C[C@H]1CC ; CCCC(c1ccc(C(F)(F)F)cc1)N1C[C@H](CC)N(c2nc(=O)n(C)c3ccc(C#N)nc23)C[C@H]1CC]

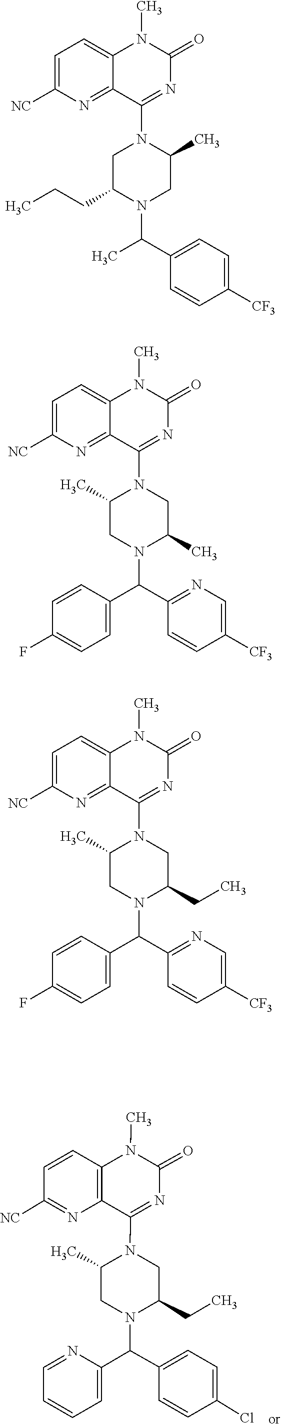 [SMILES: CCC[C@@H]1CN(c2nc(=O)n(C)c3ccc(C#N)nc23)[C@@H](C)CN1C(C)c1ccc(C(F)(F)F)cc1 ; C[C@@H]1CN(c2nc(=O)n(C)c3ccc(C#N)nc23)[C@@H](C)CN1C(c1ccc(F)cc1)c1ccc(C(F)(F)F)cn1 ; CC[C@@H]1CN(c2nc(=O)n(C)c3ccc(C#N)nc23)[C@@H](C)CN1C(c1ccc(F)cc1)c1ccc(C(F)(F)F)cn1 ; CC[C@@H]1CN(c2nc(=O)n(C)c3ccc(C#N)nc23)[C@@H](C)CN1C(c1ccc(Cl)cc1)c1ccccn1]

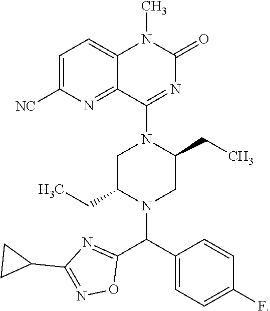 [SMILES: CC[C@H]1CN(C(c2ccc(F)cc2)c2nc(C3CC3)no2)[C@H](CC)CN1c1nc(=O)n(C)c2ccc(C#N)nc12]

61. The method of claim 57, wherein the inhibitor is a compound of Formula (II) or a pharmaceutically acceptable salt thereof having the structure:

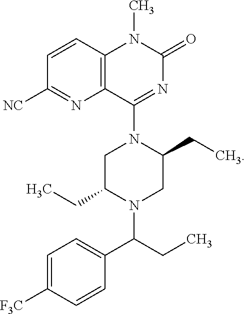 [SMILES: CCC(c1ccc(C(F)(F)F)cc1)N1C[C@H](CC)N(c2nc(=O)n(C)c3ccc(C#N)nc23)C[C@H]1CC]

62. The method of any one of claims 1-13 and 21-61, wherein the inhibitor is administered in a therapeutically effective amount.

63. The method of claim 62, wherein the therapeutically effective dose provides a dose of the inhibitor that is continuously therapeutically effective for the period of time that the inhibitor is administered.

64. The method of any one of claims 1-63, wherein the inhibitor is administered in a per day amount between or between about 0.25 and 250 mg, inclusive.

65. The method of any one of claims 1-64, wherein the inhibitor is administered in a per day amount between or between about 0.5 and 100 mg, inclusive.

66. The method of any one of claims 1-65, wherein the inhibitor is administered orally.

67. The method of any one of claims 1-66, wherein the recombinant receptor is an engineered T cell receptor (eTCR).

68. The method of any one of claims 1-66, wherein the recombinant receptor is a chimeric antigen receptor (CAR).

69. The method of claim 68, wherein the antigen is CD19, and the CAR is an anti-CD19 CAR.

70. The method of claim 68 or claim 69, wherein the CAR is the CAR of BREYANZI® (lisocabtagene maraleucel), TECARTUS™ (brexucabtagene autoleucel), KYMRIAH™ (tisagenlecleucel), or YESCARTA™ (axicabtagene ciloleucel).

71. The method of any one of claims 68-70, wherein the T cell therapy is BREYANZI® (lisocabtagene maraleucel), TECARTUS™ (brexucabtagene autoleucel), KYMRIAH™ (tisagenlecleucel), or YESCARTA™ (axicabtagene ciloleucel).

72. The method of claim 68, wherein the antigen is BCMA, and the CAR is an anti-BCMA CAR.

73. The method of claim 68 or claim 72, wherein the CAR is the CAR of ABECMA® (idecabtagene vicleucel) or CARVYKTI™ (ciltacabtagene autoleucel).

74. The method of any one of claims 68, 72, and 73, wherein the T cell therapy is ABECMA® (idecabtagene vicleucel) or CARVYKTI™ (ciltacabtagene autoleucel).

75. The method of any one of claims 1-74, wherein the cancer is a solid tumor.

76. The method of any one of claims 1-74, wherein the cancer is a hematological (liquid) tumor.

77. The method of any one of claims 1-76, wherein the cancer is a B cell malignancy.

78. The method of any one of claims 1-74, 76, and 77, wherein the cancer is a leukemia.

79. The method of any one of claims 1-74, 76, and 77, wherein the cancer is a lymphoma.

80. The method of any one of claims 1-74, 76, and 77, wherein the cancer is a myeloma.

81. The method of claim 80, wherein the myeloma is multiple myeloma.

82. The method of any one of claims 1-81, wherein the cancer is relapsed or refractory.

83. The method of any one of claims 1-82, wherein the T cell therapy comprises between or between about 0.1×10⁶ and 1,000×10⁶ total recombinant receptor-expressing T cells, inclusive.

84. The method of any one of claims 1-83, wherein the T cell therapy comprises between or between about 10×10⁶ and 1,000×10⁶ total recombinant receptor-expressing T cells, inclusive.

85. The method of any one of claims 1-84, wherein the T cell therapy comprises between or between about 10×10⁶ and 500×10⁶ total recombinant receptor-expressing T cells, inclusive.

86. The method of any of claims 83-85, wherein the recombinant receptor-expressing T cells are viable recombinant receptor-expressing T cells.

87. The method of any one of claims 1-86, wherein the T cell therapy is administered intravenously.

88. The method of any one of claims 1-87, wherein the T cells of the T cell therapy are autologous to the subject.

89. The method of any one of claims 1-87, wherein the T cells of the T cell therapy are allogeneic to the subject.

90. The method of any one of claims 1-89, wherein the T cells of the T cell therapy are human T cells.


SEQUENCE LISTING (252 sequences)

SEQ ID NO: 1 - protein, 12 aa, Homo sapiens
  REGION 1..12: note = spacer (IgG4hinge) (aa)
ESKYGPPCPPCP

SEQ ID NO: 2 - DNA, 36 bp, Homo sapiens
  misc_feature 1..36: note = spacer (IgG4hinge) (nt)
  source 1..36: mol_type = unassigned DNA
gaatctaagtacggaccgccctgccccccttgccct

SEQ ID NO: 3 - protein, 119 aa, Homo sapiens
  REGION 1..119: note = Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, Homo sapiens
  REGION 1..229: note = Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, Homo sapiens
  REGION 1..282: note = IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, synthetic construct
  REGION 1..24: note = T2A
LEGGGEGRGSLLTCGDVEENPGPR

SEQ ID NO: 7 - protein, 335 aa, synthetic construct
  REGION 1..335: note = tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, Homo sapiens
  REGION 1..27: note = CD28 (amino acids 153-179 of Accession No. P10747)
FWVLVVVGGVLACYSLLVTVAFIIFWV

SEQ ID NO: 9 - protein, 66 aa, Homo sapiens
  REGION 1..66: note = CD28 (amino acids 114-179 of Accession No. P10747)
IEVMYPPPYLDNEKSNGTIIHVKGKHLCPSPLFPGPSKPFWVLVVVGGVLACYSLLVTVAFIIFWV

SEQ ID NO: 10 - protein, 41 aa, Homo sapiens
  REGION 1..41: note = CD28 (amino acids 180-220 of P10747)
RSKRSRLLHSDYMNMTPRRPGPTRKHYQPYAPPRDFAAYRS

SEQ ID NO: 11 - protein, 41 aa, Homo sapiens
  REGION 1..41: note = CD28 (LL to GG)
RSKRSRGGHSDYMNMTPRRPGPTRKHYQPYAPPRDFAAYRS

SEQ ID NO: 12 - protein, 42 aa, Homo sapiens
  REGION 1..42: note = 4-1BB (amino acids 214-255 of Q07011.1)
KRGRKKLLYIFKQPFMRPVQTTQEEDGCSCRFPEEEEGGCEL

SEQ ID NO: 13 - protein, 112 aa, Homo sapiens
  REGION 1..112: note = CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, Homo sapiens
  REGION 1..112: note = CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Homo sapiens
  REGION 1..112: note = CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 30 aa, synthetic construct
  REGION 1..29: note = Linker
PGGGSGGGGSGGGGSGGGGSGGGGSGGGGP

SEQ ID NO: 17 - protein, 17 aa, synthetic construct
  REGION 1..17: note = Linker
GSADDAKKDAAKKDGKS

SEQ ID NO: 18 - protein, 54 aa, Homo sapiens
  REGION 1..54: note = Extracellular domain of human BCMA (GenBank No. NP_001183.2)
MLQMAGQCSQNEYFDSLLHACIPCQLRCSSNTPPLTCQRYCNASVTNSVKGTNA

SEQ ID NO: 19 - protein, 5 aa, synthetic construct
  REGION 1..5: note = Linker sequence
GGGGS

SEQ ID NO: 20 - protein, 231 aa, synthetic construct
  REGION 1..231: note = Modified Human IgG1 Fc
PKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 16 aa, synthetic construct
  REGION 1..16: note = CD33 Signal peptide
MPLLLLLPLLWAGALA

SEQ ID NO: 22 - protein, 306 aa, synthetic construct
  REGION 1..306: note = BCMA-Fc construct
MPLLLLLPLLWAGALAMLQMAGQCSQNEYFDSLLHACIPCQLRCSSNTPPLTCQRYCNASVTNSVKGTNAGGGGSPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 18 aa, synthetic construct
  REGION 1..18: note = T2A
EGRGSLLTCGDVEENPGP

SEQ ID NO: 24 - protein, 22 aa, synthetic construct
  REGION 1..22: note = P2A
GSGATNFSLLKQAGDVEENPGP

SEQ ID NO: 25 - protein, 19 aa, synthetic construct
  REGION 1..19: note = P2A
ATNFSLLKQAGDVEENPGP

SEQ ID NO: 26 - protein, 20 aa, synthetic construct
  REGION 1..20: note = E2A
QCTNYALLKLAGDVESNPGP

SEQ ID NO: 27 - protein, 22 aa, synthetic construct
  REGION 1..22: note = F2A
VKQTLNFDLLKLAGDVESNPGP

SEQ ID NO: 28 - protein, 335 aa, synthetic construct; identical to SEQ ID NO: 7
  REGION 1..335: note = tEGFR

SEQ ID NO: 29 - protein, 228 aa, Homo sapiens
  REGION 1..228: note = Hinge-CH2-CH3 spacer
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 117 aa, synthetic construct
  REGION 1..117: note = Variable heavy (VH) Anti-BCMA
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 31 - protein, 111 aa, synthetic construct
  REGION 1..111: note = Variable light (VL) Anti-BCMA
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 32 - protein, 122 aa, synthetic construct
  REGION 1..122: note = Variable heavy (VH) Anti-BCMA
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSA

SEQ ID NO: 33 - protein, 107 aa, synthetic construct
  REGION 1..107: note = Variable light (VL) Anti-BCMA
DVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIK

SEQ ID NO: 34 - protein, 116 aa, synthetic construct
  REGION 1..116: note = Variable heavy (VH) Anti-BCMA
EVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSS

SEQ ID NO: 35 - protein, 111 aa, synthetic construct
  REGION 1..111: note = Variable light (VL) Anti-BCMA
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLG

SEQ ID NO: 36 - protein, 118 aa, synthetic construct
  REGION 1..118: note = Variable heavy (VH) Anti-BCMA
EVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 37 - protein, 105 aa, synthetic construct
  REGION 1..105: note = Variable light (VL) Anti-BCMA
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLG

SEQ ID NO: 38 - protein, 122 aa, synthetic construct
  REGION 1..122: note = Variable heavy (VH) Anti-BCMA
EVQLVQSGAEVKKPGSSVKVSCKASGGTFSSYAISWVRQAPGQGLEWMGRIIPILGIANYAQKFQGRVTMTEDTSTDTAYMELSSLRSEDTAVYYCARSGYSKSIVSYMDYWGQGTLVTVSS

SEQ ID NO: 39 - protein, 111 aa, synthetic construct
  REGION 1..111: note = Variable light (VL) Anti-BCMA
LPVLTQPPSTSGTPGQRVTVSCSGSSSNIGSNVVFWYQQLPGTAPKLVIYRNNQRPSGVPDRFSVSKSGTSASLAISGLRSEDEADYYCAAWDDSLSGYVFGTGTKVTVLG

SEQ ID NO: 40 - protein, 120 aa, synthetic construct
  REGION 1..120: note = Variable heavy (VH) Anti-BCMA
QVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSS

SEQ ID NO: 41 - protein, 112 aa, synthetic construct
  REGION 1..112: note = Variable light (VL) Anti-BCMA
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLG

SEQ ID NO: 42 - protein, 118 aa, synthetic construct
  REGION 1..118: note = Variable heavy (VH) Anti-BCMA
QVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSS

SEQ ID NO: 43 - protein, 112 aa, synthetic construct
  REGION 1..112: note = Variable light (VL) Anti-BCMA
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLG

SEQ ID NO: 44 - protein, 5 aa, synthetic construct
  REGION 1..5: note = FMC63 CDR H1
DYGVS

SEQ ID NO: 45 - protein, 16 aa, synthetic construct
  REGION 1..16: note = FMC63 CDR H2
VIWGSETTYYNSALKS

SEQ ID NO: 46 - protein, 7 aa, synthetic construct
  REGION 1..7: note = FMC63 CDR H3
YAMDYWG

SEQ ID NO: 47 - protein, 12 aa, synthetic construct
  REGION 1..12: note = FMC63 HC-CDR3
HYYYGGSYAMDY

SEQ ID NO: 48 - protein, 11 aa, synthetic construct
  REGION 1..11: note = FMC63 CDR L1
RASQDISKYLN

SEQ ID NO: 49 - protein, 7 aa, synthetic construct
  REGION 1..7: note = FMC63 CDR L2
SRLHSGV

SEQ ID NO: 50 - protein, 7 aa, synthetic construct
  REGION 1..7: note = FMC63 LC-CDR2
HTSRLHS

SEQ ID NO: 51 - protein, 9 aa, synthetic construct
  REGION 1..9: note = FMC63 CDR L3
GNTLPYTFG

SEQ ID NO: 52 - protein, 9 aa, synthetic construct
  REGION 1..9: note = FMC63 LC-CDR3
QQGNTLPYT

SEQ ID NO: 53 - protein, 120 aa, synthetic construct
  REGION 1..120: note = FMC63 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 54 - protein, 107 aa, synthetic construct
  REGION 1..107: note = FMC63 VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 55 - protein, 245 aa, synthetic construct
  REGION 1..245: note = FMC63 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 56 - protein, 11 aa, synthetic construct
  REGION 1..11: note = SJ25C1 CDR L1
KASQNVGTNVA

SEQ ID NO: 57 - protein, 7 aa, synthetic construct
  REGION 1..7: note = SJ25C1 CDR L2
SATYRNS

SEQ ID NO: 58 - protein, 9 aa, synthetic construct
  REGION 1..9: note = SJ25C1 CDR L3
QQYNRYPYT

SEQ ID NO: 59 - protein, 5 aa, synthetic construct
  REGION 1..5: note = SJ25C1 CDR H1
SYWMN

SEQ ID NO: 60 - protein, 17 aa, synthetic construct
  REGION 1..17: note = SJ25C1 CDR H2
QIYPGDGDTNYNGKFKG

SEQ ID NO: 61 - protein, 13 aa, synthetic construct
  REGION 1..13: note = SJ25C1 CDR H3
KTISSVVDFYFDY

SEQ ID NO: 62 - protein, 122 aa, synthetic construct
  REGION 1..122: note = SJ25C1 VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 63 - protein, 108 aa, synthetic construct
  REGION 1..108: note = SJ25C1 VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 64 - protein, 15 aa, synthetic construct
  REGION 1..15: note = Linker
GGGGSGGGGSGGGGS

SEQ ID NO: 65 - protein, 245 aa, synthetic construct
  REGION 1..245: note = SJ25C1 scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 66 - protein, 12 aa, synthetic construct
  REGION 1..12: note = FMC63 HC-CDR3
HYYYGGSYAMDY

SEQ ID NO: 67 - protein, 7 aa, synthetic construct
  REGION 1..7: note = FMC63 LC-CDR2
HTSRLHS

SEQ ID NO: 68 - protein, 9 aa, synthetic construct
  REGION 1..9: note = FMC63 LC-CDR3
QQGNTLPYT

SEQ ID NO: 69 - DNA, 735 bp, synthetic construct
  misc_feature 1..735: note = Sequence encoding scFv
  source 1..735: mol_type = other DNA
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 70 - protein, 18 aa, synthetic construct
  REGION 1..18: note = Linker
GSTSGSGKPGSGEGSTKG

SEQ ID NO: 71 - protein, 4 aa, synthetic construct
  REGION 1..4: note = Linker
GGGS

SEQ ID NO: 72 - protein, 15 aa, synthetic construct
  REGION 1..15: note = Linker
GGGGSGGGGSGGGGS

SEQ ID NO: 73 - protein, 18 aa, synthetic construct
  REGION 1..18: note = Linker
GSTSGSGKPGSGEGSTKG

SEQ ID NO: 74 - protein, 21 aa, synthetic construct
  REGION 1..21: note = Linker
SRGGGGSGGGGSGGGGSLEMA

SEQ ID NO: 75 - protein, 21 aa, synthetic construct
  REGION 1..21: note = CD8a signal peptide
MALPVTALLLPLALLLHAARP

SEQ ID NO: 76 - protein, 20 aa, synthetic construct
  REGION 1..20: note = Signal peptide
METDTLLLWVLLLWVPGSTG

SEQ ID NO: 77 - protein, 118 aa, synthetic construct
  REGION 1..118: note = Variable heavy (VH) Anti-BCMA
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSS

SEQ ID NO: 78 - protein, 107 aa, synthetic construct
  REGION 1..107: note = Variable light (VL) Anti-BCMA
EIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIK

SEQ ID NO: 79 - protein, 122 aa, synthetic construct
  REGION 1..122: note = Variable heavy (VH) Anti-BCMA
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSS

SEQ ID NO: 80 - protein, 112 aa, synthetic construct
  REGION 1..112: note = Variable light (VL) Anti-BCMA
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIK

SEQ ID NO: 81 - protein, 116 aa, synthetic construct
  REGION 1..116: note = Variable heavy (VH) Anti-BCMA
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSS

SEQ ID NO: 82 - protein, 106 aa, synthetic construct
  REGION 1..106: note = Variable light (VL) Anti-BCMA
EIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIK

SEQ ID NO: 83 - protein, 122 aa, synthetic construct
  REGION 1..122: note = Variable heavy (VH) Anti-BCMA
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSS

SEQ ID NO: 84 - protein, 107 aa, synthetic construct
  REGION 1..107: note = Variable light (VL) Anti-BCMA
EIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIK

SEQ ID NO: 85 - protein, 119 aa, synthetic construct
  REGION 1..119: note = Variable heavy (VH) Anti-BCMA
EVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSS

SEQ ID NO: 86 - protein, 107 aa, synthetic construct
  REGION 1..107: note = Variable light (VL) Anti-BCMA
EIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIK

SEQ ID NO: 87 - protein, 122 aa, synthetic construct
  REGION 1..122: note = Variable heavy (VH) Anti-BCMA
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSS

SEQ ID NO: 88 - protein, 107 aa, synthetic construct
  REGION 1..107: note = Variable light (VL) Anti-BCMA
DIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIK

SEQ ID NO: 89 - protein, 126 aa, synthetic construct
  REGION 1..126: note = Variable heavy (VH) Anti-BCMA
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSS

SEQ ID NO: 90 - protein, 113 aa, synthetic construct
  REGION 1..113: note = Variable light (VL) Anti-BCMA
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIK

SEQ ID NO: 91 - protein, 123 aa, synthetic construct
  REGION 1..123: note = Variable heavy (VH) Anti-BCMA
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSS

SEQ ID NO: 92 - protein, 107 aa, synthetic construct
  REGION 1..107: note = Variable light (VL) Anti-BCMA
EIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIK

SEQ ID NO: 93 - protein, 121 aa, synthetic construct
  REGION 1..121: note = Variable heavy (VH) Anti-BCMA
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSS

SEQ ID NO: 94 - protein, 112 aa, synthetic construct
  REGION 1..112: note = Variable light (VL) Anti-BCMA
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLG

SEQ ID NO: 95 - protein, 117 aa, synthetic construct
  REGION 1..117: note = Variable heavy (VH) Anti-BCMA
QVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 96 - protein, 111 aa, synthetic construct
  REGION 1..111: note = Variable light (VL) Anti-BCMA
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIK

SEQ ID NO: 97 - protein, 115 aa, synthetic construct
  REGION 1..115: note = Variable heavy (VH) Anti-BCMA
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 98 - protein, 107 aa, synthetic construct
  REGION 1..107: note = Variable light (VL) Anti-BCMA
DIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 99 - protein, 120 aa, synthetic construct
  REGION 1..120: note = Variable heavy (VH) Anti-BCMA
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSS

SEQ ID NO: 100 - protein, 109 aa, synthetic construct
  REGION 1..109: note = Variable light (VL) Anti-BCMA
DIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIK

SEQ ID NO: 101 - protein, 115 aa, synthetic construct
  REGION 1..115: note = Variable heavy (VH) Anti-BCMA
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 102 - protein, 107 aa, synthetic construct
  REGION 1..107: note = Variable light (VL) Anti-BCMA
DIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIK

SEQ ID NO: 103 - protein, 115 aa, synthetic construct; identical to SEQ ID NO: 97
  REGION 1..115: note = Variable heavy (VH) Anti-BCMA

SEQ ID NO: 104 - protein, 109 aa, synthetic construct
  REGION 1..109: note = Variable light (VL) Anti-BCMA
EIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIK

SEQ ID NO: 105 - protein, 117 aa, synthetic construct
  REGION 1..117: note = Variable heavy (VH) Anti-BCMA
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 106 - protein, 111 aa, synthetic construct
  REGION 1..111: note = Variable light (VL) Anti-BCMA
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 107 - protein, 117 aa, synthetic construct
  REGION 1..117: note = Variable heavy (VH) Anti-BCMA
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSS

SEQ ID NO: 108 - protein, 111 aa, synthetic construct
  REGION 1..111: note = Variable light (VL) Anti-BCMA
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 109 - protein, 119 aa, synthetic construct
  REGION 1..119: note = Variable heavy (VH) Anti-BCMA
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSS

SEQ ID NO: 110 - protein, 112 aa, synthetic construct
  REGION 1..112: note = Variable light (VL) Anti-BCMA
DIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIK

SEQ ID NO: 111 - protein, 119 aa, synthetic construct
  REGION 1..119: note = Variable heavy (VH) Anti-BCMA
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSS

SEQ ID NO: 112 - protein, 112 aa, synthetic construct
  REGION 1..112: note = Variable light (VL) Anti-BCMA
DIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIK

SEQ ID NO: 113 - protein, 122 aa, synthetic construct
  REGION 1..122: note = Anti-BCMA sdAb
QVQLVESGGGLVQPGGSLRLSCEASGFTLDYYAIGWFRQAPGKEREGVICISRSDGSTYYADSVKGRFTISRDNAKKTVYLQMISLKPEDTAAYYCAAGADCSGYLRDYEFRGQGTQVTVSS

SEQ ID NO: 114 - protein, 39 aa, synthetic construct
  REGION 1..39: note = CD28 spacer
IEVMYPPPYLDNEKSNGTIIHVKGKHLCPSPLFPGPSKP

SEQ ID NO: 115 - protein, 25 aa, synthetic construct
  REGION 1..25: note = CD8a TM
IYIWAPLAGTCGVLLLSLVITLYCN

SEQ ID NO: 116 - protein, 30 aa, synthetic construct
  REGION 1..30: note = CD28 spacer (truncated)
LDNEKSNGTIIHVKGKHLCPSPLFPGPSKP

SEQ ID NO: 117 - protein, 46 aa, synthetic construct
  REGION 1..46: note = CD8a hinge
PTTTPAPRPPTPAPTIASQPLSLRPEACRPAAGGAVHTRGLDFACD

SEQ ID NO: 118 - protein, 45 aa, synthetic construct
  REGION 1..45: note = CD8a hinge
TTTPAPRPPTPAPTIASQPLSLRPEACRPAAGGAVHTRGLDFACD

SEQ ID NO: 119 - protein, 55 aa, synthetic construct
  REGION 1..55: note = CD8a hinge
FVPVFLPAKPTTTPAPRPPTPAPTIASQPLSLRPEACRPAAGGAVHTRGLDFACD

SEQ ID NO: 120 - protein, 39 aa, synthetic construct
  REGION 1..39: note = CTLA4 hinge
DTGLYICKVELMYPPPYYLGIGNGTQIYVIDPEPCPDSD

SEQ ID NO: 121 - protein, 24 aa, synthetic construct
  REGION 1..24: note = CTLA4 TM
FLLWILAAVSSGLFFYSFLLTAVS

SEQ ID NO: 122 - protein, 38 aa, synthetic construct
  REGION 1..38: note = PD-1 hinge
QIKESLRAELRVTERRAEVPTAHPSPSPRPAGQFQTLV

SEQ ID NO: 123 - protein, 21 aa, synthetic construct
  REGION 1..21: note = PD-1 TM
VGVVGGLLGSLVLLVWVLAVI

SEQ ID NO: 124 - protein, 16 aa, synthetic construct
  REGION 1..16: note = FcyRIIIa hinge
GLAVSTISSFFPPGYQ

SEQ ID NO: 125 - protein, 231 aa, synthetic construct
  REGION 1..231: note = IgG1 hinge
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 457 aa, synthetic construct
  REGION 1..457: note = anti-BCMA CAR
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 457 aa, synthetic construct
  REGION 1..457: note = anti-BCMA CAR
EIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIKRGSTSGSGKPGSGEGSTKGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 466 aa, synthetic construct
  REGION 1..466: note = anti-BCMA CAR
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSSGSTSGSGKPGSGEGSTKGDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 466 aa, synthetic construct
  REGION 1..466: note = anti-BCMA CAR
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 454 aa, synthetic construct
  REGION 1..454: note = anti-BCMA CAR
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSSGSTSGSGKPGSGEGSTKGEIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 454 aa, synthetic construct
  REGION 1..454: note = anti-BCMA CAR
EIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIKRGSTSGSGKPGSGEGSTKGQVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 461 aa, synthetic construct
  REGION 1..461: note = anti-BCMA CAR
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSSGSTSGSGKPGSGEGSTKGEIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 461 aa, synthetic construct
  REGION 1..461: note = anti-BCMA CAR
EIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIKRGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 458 aa, synthetic construct
  REGION 1..458: note = anti-BCMA CAR
EVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 458 aa, synthetic construct
  REGION 1..458: note = anti-BCMA CAR
EIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIKRGSTSGSGKPGSGEGSTKGEVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 136 - protein, 461 aa, synthetic construct
  REGION 1..461: note = anti-BCMA CAR
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSSGSTSGSGKPGSGEGSTKGDIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 461 aa, synthetic construct
  REGION 1..461: note = anti-BCMA CAR
DIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 471 aa, synthetic construct
  REGION 1..471: note = anti-BCMA CAR
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSSGSTSGSGKPGSGEGSTKGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 471 aa, synthetic construct
  REGION 1..471: note = anti-BCMA CAR
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIKRGSTSGSGKPGSGEGSTKGQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 462 aa, synthetic construct
  REGION 1..462: note = anti-BCMA CAR
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSSGSTSGSGKPGSGEGSTKGEIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 462 aa, synthetic construct
  REGION 1..462: note = anti-BCMA CAR
EIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - protein, 466 aa, synthetic construct
  REGION 1..466: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - protein, 473 aa, synthetic construct
  REGION 1..473: note = anti-BCMA CAR
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLGSRGGGGSGGGGSGGGGSLEMAQVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 144 - protein, 470 aa, synthetic construct
  REGION 1..470: note = anti-BCMA CAR
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 145 - protein, 476 aa, synthetic construct
  REGION 1..476: note = anti-BCMA CAR
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLGSRGGGGSGGGGSGGGGSLEMAQVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 146 - protein, 475 aa, synthetic construct
  REGION 1..475: note = anti-BCMA CAR
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLGSRGGGGSGGGGSGGGGSLEMAQVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - protein, 482 aa, synthetic construct
  REGION 1..482: note = anti-BCMA CAR
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLGSRGGGGSGGGGSGGGGSLEMAQVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 148 - protein, 476 aa, synthetic construct
  REGION 1..476: note = anti-BCMA CAR
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 149 - protein, 481 aa, synthetic construct
  REGION 1..481: note = anti-BCMA CAR
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLGSRGGGGSGGGGSGGGGSLEMAQVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 150 - protein, 479 aa, synthetic construct
  REGION 1..479: note = anti-BCMA CAR
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLGSRGGGGSGGGGSGGGGSLEMAQVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 151 - protein, 472 aa, synthetic construct
  REGION 1..472: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 152 - protein, 472 aa, synthetic construct
  REGION 1..472: note = anti-BCMA CAR
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 153 - protein, 472 aa, synthetic construct
  REGION 1..472: note = anti-BCMA CAR
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 154 - protein, 466 aa, synthetic construct
  REGION 1..466: note = anti-BCMA CAR
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAADTGLYICKVELMYPPPYYLGIGNGTQIYVIDPEPCPDSDFLLWILAAVSSGLFFYSFLLTAVSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 155 - protein, 464 aa, synthetic construct
  REGION 1..464: note = anti-BCMA CAR
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAAQIKESLRAELRVTERRAEVPTAHPSPSPRPAGQFQTLVVGVVGGLLGSLVLLVWVLAVICSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 156 - protein, 653 aa, synthetic construct
  REGION 1..653: note = anti-BCMA CAR
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGDYTEDYWGQGTLVTVSSGGGGSGGGGSGGGGSQSALTQPASVSGSPGQSITISCTGSSSDVGKYNLVSWYQQPPGKAPKLIIYDVNKRPSGVSNRFSGSKSGNTATLTISGLQGDDEADYYCSSYGGSRSYVFGTGTKVTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 157 - protein, 650 aa, synthetic construct
  REGION 1..650: note = anti-BCMA CAR
EVQLVQSGGGLVQPGRSLRLSCTASGFTFGDYAMSWFKQAPGKGLEWVGFIRSKAYGGTTEYAASVKGRFTISRDDSKSIAYLQMNSLKTEDTAVYYCAAWSAPTDYWGQGTLVTVSSGGGGSGGGGSGGGGSDIQMTQSPAFLSASVGDRVTVTCRASQGISNYLAWYQQKPGNAPRLLIYSASTLQSGVPSRFRGTGYGTEFSLTIDSLQPEDFATYYCQQSYTSRQTFGPGTRLDIKESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 158 - protein, 651 aa, synthetic construct
  REGION 1..651: note = anti-BCMA CAR
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGPPSFDIWGQGTMVTVSSGGGGSGGGGSGGGGSSYVLTQPPSVSVAPGQTARITCGANNIGSKSVHWYQQKPGQAPMLVVYDDDDRPSGIPERFSGSNSGNTATLTISGVEAGDEADYFCHLWDRSRDHYVFGTGTKLTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 159 - protein, 658 aa, synthetic construct
  REGION 1..658: note = anti-BCMA CAR
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 160 - protein, 654 aa, synthetic construct
  REGION 1..654: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 161 - protein, 653 aa, synthetic construct
  REGION 1..653: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 162 - protein, 462 aa, synthetic construct
  REGION 1..462: note = anti-BCMA CAR
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 163 - protein, 469 aa, synthetic construct
  REGION 1..469: note = anti-BCMA CAR
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSSASGGGGSGGRASGGGGSDIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 164 - protein, 462 aa, synthetic construct
  REGION 1..462: note = anti-BCMA CAR
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 165 - protein, 464 aa, synthetic construct
  REGION 1..464: note = anti-BCMA CAR
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 166 - protein, 467 aa, synthetic construct
  REGION 1..467: note = anti-BCMA CAR
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSAGGGGSGGGGSGGGGSDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 167 - protein, 472 aa, synthetic construct
  REGION 1..472: note = anti-BCMA CAR
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 168 - protein, 466 aa, synthetic construct
  REGION 1..466: note = anti-BCMA CAR
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 169 - protein, 466 aa, synthetic construct
  REGION 1..466: note = anti-BCMA CAR
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 170 - protein, 466 aa, synthetic construct
  REGION 1..466: note = anti-BCMA CAR
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 171 - protein, 482 aa, synthetic construct
  REGION 1..482: note = anti-BCMA CAR
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 172 - protein, 440 aa, synthetic construct
  REGION 1..440: note = anti-BCMA CAR
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 173 - protein, 469 aa, synthetic construct
  REGION 1..469: note = anti-BCMA CAR
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 174 - protein, 655 aa, synthetic construct
  REGION 1..655: note = anti-BCMA CAR
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 175 - protein, 440 aa, synthetic construct
  REGION 1..440: note = anti-BCMA CAR
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 176 - protein, 469 aa, synthetic construct
  REGION 1..469: note = anti-BCMA CAR
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 177 - protein, 655 aa, synthetic construct
  REGION 1..655: note = anti-BCMA CAR
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 178 - protein, 28 aa, synthetic construct
  REGION 1..28: note = CD8a TM
IYIWAPLAGTCGVLLLSLVITLYCNHRN

SEQ ID NO: 179 - protein, 21 aa, synthetic construct
  REGION 1..21: note = CD8a TM
IYIWAPLAGTCGVLLLSLVIT

SEQ ID NO: 180 - protein, 4 aa, synthetic construct
  REGION 1..4: note = Linking peptide
RAAA

SEQ ID NO: 181 - protein, 118 aa, synthetic construct
  REGION 1..118: note = Variable heavy (VH) Anti-BCMA
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGDYTEDYWGQGTLVTVSS

SEQ ID NO: 182 - protein, 110 aa, synthetic construct
  REGION 1..110: note = Variable light (VL) Anti-BCMA
QSALTQPASVSGSPGQSITISCTGSSSDVGKYNLVSWYQQPPGKAPKLIIYDVNKRPSGVSNRFSGSKSGNTATLTISGLQGDDEADYYCSSYGGSRSYVFGTGTKVTVL

SEQ ID NO: 183 - protein, 118 aa, synthetic construct
  REGION 1..118: note = Variable heavy (VH) Anti-BCMA
EVQLVQSGGGLVQPGRSLRLSCTASGFTFGDYAMSWFRQAPGKGLEWVGFIRSKAYGGTTEYAASVKGRFTISRDDSKSIAYLQMNSLKTEDTAVYYCAAWSAPTDYWGQGTLVTVSS

SEQ ID NO: 184 - protein, 107 aa, synthetic construct
  REGION 1..107: note = Variable light (VL) Anti-BCMA
DIQMTQSPAFLSASVGDRVTVTCRASQGISNYLAWYQQKPGNAPRLLIYSASTLQSGVPSRFRGTGYGTEFSLTIDSLQPEDFATYYCQQSYTSRQTFGPGTRLDIK

SEQ ID NO: 185 - protein, 118 aa, synthetic construct
  REGION 1..118: note = Variable heavy (VH) Anti-BCMA
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGPPSFDIWGQGTMVTVSS

SEQ ID NO: 186 - protein, 108 aa, synthetic construct
  REGION 1..108: note = Variable light (VL) Anti-BCMA
SYVLTQPPSVSVAPGQTARITCGANNIGSKSVHWYQQKPGQAPMLVVYDDDDRPSGIPERFSGSNSGNTATLTISGVEAGDEADYFCHLWDRSRDHYVFGTGTKLTVL

SEQ ID NO: 187 - protein, 122 aa, synthetic construct; identical to SEQ ID NO: 38
  REGION 1..122: note = Variable heavy (VH) Anti-BCMA

SEQ ID NO: 188 - protein, 111 aa, synthetic construct; identical to SEQ ID NO: 39
  REGION 1..111: note = Variable light (VL) Anti-BCMA

SEQ ID NO: 189 - protein, 17 aa, synthetic construct
  REGION 1..17: note = Linker
ASGGGGSGGRASGGGGS

SEQ ID NO: 190 - protein, 751 aa, Homo sapiens
  REGION 1..751: note = DGKalpha
MAKERGLISPSDFAQLQKYMEYSTKKVSDVLKLFEDGEMAKYVQGDAIGYEGFQQFLKIYLEVDNVPRHLSLALFQSFETGHCLNETNVTKDVVCLNDVSCYFSLLEGGRPEDKLEFTFKLYDTDRNGILDSSEVDKIILQMMRVAEYLDWDVSELRPILQEMMKEIDYDGSGSVSQAEWVRAGATTVPLLVLLGLEMTLKDDGQHMWRPKRFPRPVYCNLCESSIGLGKQGLSCNLCKYTVHDQCAMKALPCEVSTYAKSRKDIGVQSHVWVRGGCESGRCDRCQKKIRIYHSLTGLHCVWCHLEIHDDCLQAVGHECDCGLLRDHILPPSSIYPSVLASGPDRKNSKTSQKTMDDLNLSTSEALRIDPVPNTHPLLVFVNPKSGGKQGQRVLWKFQYILNPRQVFNLLKDGPEIGLRLFKDVPDSRILVCGGDGTVGWILETIDKANLPVLPPVAVLPLGTGNDLARCLRWGGGYEGQNLAKILKDLEMSKVVHMDRWSVEVIPQQTEEKSDPVPFQIINNYFSIGVDASIAHRFHIMREKYPEKFNSRMKNKLWYFEFATSESIFSTCKKLEESLTVEICGKPLDLSNLSLEGIAVLNIPSMHGGSNLWGDTRRPHGDIYGINQALGATAKVITDPDILKTCVPDLSDKRLEVVGLEGAIEMGQIYTKLKNAGRRLAKCSEITFHTTKTLPMQIDGEPWMQTPCTIKITHKNQMPMLMGPPPRSTNFFGFLSGSSETVRFQGHHHHHH

SEQ ID NO: 191 - protein, 944 aa, Homo sapiens
  REGION 1..944: note = DGKzeta
MEPRDGSPEARSSDSESASASSSGSERDAGPEPDKAPRRLNKRRFPGLRLFGHRKAITKSGLQHLAPPPPTPGAPCSESERQIRSTVDWSESATYGEHIWFETNVSGDFCYVGEQYCVARLQKSVSRRKCAACKIVVHTPCIEQLEKINFRCKPSFRESGSRNVREPTFVRHHWVHRRRQDGKCRHCGKGFQQKFTFHSKEIVAISCSWCKQAYHSKVSCFMLQQIEEPCSLGVHAAVVIPPTWILRARRPQNTLKASKKKKRASFKRKSSKKGPEEGRWRPFIIRPTPSPLMKPLLVFVNPKSGGNQGAKIIQSFLWYLNPRQVFDLSQGGPKEALEMYRKVHNLRILACGGDGTVGWILSTLDQLRLKPPPPVAILPLGTGNDLARTLNWGGGYTDEPVSKILSHVEEGNVVQLDRWDLHAEPNPEAGPEDRDEGATDRLPLDVFNNYFSLGFDAHVTLEFHESREANPEKFNSRFRNKMFYAGTAFSDFLMGSSKDLAKHIRVVCDGMDLTPKIQDLKPQCVVFLNIPRYCAGTMPWGHPGEHHDFEPQRHDDGYLEVIGFTMTSLAALQVGGHGERLTQCREVVLTTSKAIPVQVDGEPCKLAASRIRIALRNQATMVQKAKRRSAAPLHSDQQPVPEQLRIQVSRVSMHDYEALHYDKEQLKEASVPLGTVVVPGDSDLELCRAHIERLQQEPDGAGAKSPTCQKLSPKWCFLDATTASRFYRIDRAQEHLNYVTEIAQDEIYILDPELLGASARPDLPTPTSPLPTSPCSPTPRSLQGDAAPPQGEELIEAAKRNDFCKLQELHRAGGDLMHRDEQSRTLLHHAVSTGSKDVVRYLLDHAPPEILDAVEENGETCLHQAAALGQRTICHYIVEAGASLMKTDQQGDTPRQRAEKAQDTELAAYLENRQHYQMIQREDQETAVGSSETVRFQGHHHHHH

SEQ ID NO: 192 - protein, 9 aa, Homo sapiens
  REGION 1..9: note = HPV E6(18-26)
KLPQLCTEL

SEQ ID NO: 193 - protein, 10 aa, Homo sapiens
  REGION 1..10: note = HPV E6(29-38)
TIHDIILECV

SEQ ID NO: 194 - protein, 9 aa, Homo sapiens
  REGION 1..9: note = HPV E6(52-60)
FAFRDLCIV

SEQ ID NO: 195 - protein, 8 aa, Homo sapiens
  REGION 1..8: note = HPV E7(86-93)
TLGIVCPI

SEQ ID NO: 196 - protein, 9 aa, Homo sapiens
  REGION 1..9: note = HPV E7(11-19)
YMLDLQPET

SEQ ID NO: 197 - protein, 9 aa, Homo sapiens
  REGION 1..9: note = HPV E7(85-93)
GTLGIVCPI

SEQ ID NO: 198 - protein, 9 aa, Homo sapiens
  REGION 1..9: note = HPV E7(82-90)
LLMGTLGIV

SEQ ID NO: 199 - protein, 9 aa, Homo sapiens
  REGION 1..9: note = HPV E7(7-15)
TLHEYMLDL

SEQ ID NO: 200 - DNA, 810 bp, synthetic construct
  misc_feature 1..810: note = TCR 57 Alpha Var. + Constant
  source 1..810: mol_type = other DNA
atggtgctgaagttttctgtgagcatcctgtggattcagctggcctgggtgtccacccagctcctggagcagagcccccagttcctgtccatccaggagggcgagaacctgaccgtgtactgcaacagctcttctgtcttttcatcactgcagtggtatagacaagaaccgggtgaaggtccagttctgctggtgaccgtcgtcaccggcggcgaggtgaagaagctaaagcgcctgacgttccagttcggagacgcgcggaaggactcgtcgctgcacatcaccgccgcccagcccggcgacaccggcctgtacctgtgcgctggcgcgcgcaacttcaacaagttctacttcggcagcggcaccaagctgaacgtgaaaccgaatatccagaatccggaccccgcggtatatcaactgcgcgactcaaaatcatccgataagagtgtctgtttgtttactgacttcgacagtcaaactaatgtctctcagagcaaagattccgatgtctacatcactgacaagtgcgttctggatatgcggagcatggattttaagtccaactccgccgtagcctggtccaacaagtcagactttgcctgtgcaaatgctttcaacaactcaattatccctgaggacactttctttccttcaccggagtcctcatgcgatgttaaactggtcgaaaaatcttttgagacggatacgaacctcaacttccaaaatttgagcgttattggctttaggattctgcttctcaaggttgcggggttcaatctcctgatgacgttgcggctttggagcagctaa

SEQ ID NO: 201 - protein, 251 aa, synthetic construct
  REGION 1..251: note = TCR 57 Alpha Var. + Constant
TQLLEQSPQFLSIQEGENLTVYCNSSSVFSSLQWYRQEPGEGPVLLVTVVTGGEVKKLKRLTFQFGDARKDSSLHITAAQPGDTGLYLCAGARNFNKFYFGSGTKLNVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 202 - protein, 110 aa, synthetic construct
  REGION 1..110: note = TCR 57 Alpha Var.
TQLLEQSPQFLSIQEGENLTVYCNSSSVFSSLQWYRQEPGEGPVLLVTVVTGGEVKKLKRLTFQFGDARKDSSLHITAAQPGDTGLYLCAGARNFNKFYFGSGTKLNVKP

SEQ ID NO: 203 - protein, 5 aa, synthetic construct
  REGION 1..5: note = TCR 57 Alpha CDR1
SVFSS

SEQ ID NO: 204 - protein, 7 aa, synthetic construct
  REGION 1..7: note = TCR 57 Alpha CDR2
VVTGGEV

SEQ ID NO: 205 - protein, 10 aa, synthetic construct
  REGION 1..10: note = TCR 57 Alpha CDR3
AGARNFNKFY

SEQ ID NO: 206 - DNA, 927 bp, synthetic construct
  misc_feature 1..927: note = TCR 57 Beta Var. + Constant
  source 1..927: mol_type = other DNA
atgctcctgctgcttctgctcctcgggcccggcagcggtctcggcgccgtcgtgtcgcagcacccgtcgtgggtgatttgcaagagcggaacgagcgtgaagatcgagtgccgctcgctcgacttccaggccacaaccatgttctggtaccgccagtttcccaagcagagcctcatgctgatggccacaagcaatgaaggcagcaaggccacttatgaacagggcgtggagaaggacaagtttctgatcaaccacgcctccctgaccctgtccaccctcactgtgaccagcgcccaccctgaggacagcagcttctacatctgcagcgcccgcagctggaggggcggcctggagcagttcttcggccctggcacccggctgacagtgctggaagatctgaagaacgtcttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcagaggc

SEQ ID NO: 207 - protein, 295 aa, synthetic construct
  REGION 1..295: note = TCR 57 Beta Var. + Constant
GAVVSQHPSWVICKSGTSVKIECRSLDFQATTMFWYRQFPKQSLMLMATSNEGSKATYEQGVEKDKFLINHASLTLSTLTVTSAHPEDSSFYICSARSWRGGLEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 208 - protein, 116 aa, synthetic construct
  REGION 1..116: note = TCR 57 Beta Var.
GAVVSQHPSWVICKSGTSVKIECRSLDFQATTMFWYRQFPKQSLMLMATSNEGSKATYEQGVEKDKFLINHASLTLSTLTVTSAHPEDSSFYICSARSWRGGLEQFFGPGTRLTVL

SEQ ID NO: 209 - protein, 6 aa, synthetic construct
  REGION 1..6: note = TCR 57 Beta CDR1
DFQATT

SEQ ID NO: 210 - protein, 7 aa, synthetic construct
  REGION 1..7: note = TCR 57 Beta CDR2
SNEGSKA

SEQ ID NO: 211 - protein, 12 aa, synthetic construct
  REGION 1..12: note = TCR 57 Beta CDR3
SARSWRGGLEQF

SEQ ID NO: 212 - DNA, 1800 bp, synthetic construct
  misc_feature 1..1800: note = TCR 57 Beta-P2A-Alpha
  source 1..1800: mol_type = other DNA
atgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagatcttggcgggggggccttgagcagttcttcgggccagggacacggctcaccgtgctagaagatctgaagaacgtcttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcagaggcggaagcggcgccacaaacttctcactgctgaaacaggctggcgacgtggaggagaatcctggcccaatggtcctgaaattctccgtgtccattctttggattcagttggcatgggtgagcacccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcagcccagcctggtgatacaggcctctacctctgtgcaggagctcgcaacttcaacaaattttactttggatctgggaccaaactcaatgtaaaaccaaatatccagaatccggaccccgcggtatatcaactgcgcgactcaaaatcatccgataagagtgtctgtttgtttactgacttcgacagtcaaactaatgtctctcagagcaaagattccgatgtctacatcactgacaagtgcgttctggatatgcggagcatggattttaagtccaactccgccgtagcctggtccaacaagtcagactttgcctgtgcaaatgctttcaacaactcaattatccctgaggacactttctttccttcaccggagtcctcatgcgatgttaaactggtcgaaaaatcttttgagacggatacgaacctcaacttccaaaatttgagcgttattggctttaggattctgcttctcaaggttgcggggttcaatctcctgatgacgttgcggctttggagcagc

SEQ ID NO: 213 - DNA, 390 bp, synthetic construct
  misc_feature 1..390: note = TCR 37 Alpha Var. + Constant
  source 1..390: mol_type = other DNA
atgaagaaactgctggccatgatcctgtggctgcagctggatagactgagcggcgagctgaaggtggaacagaaccctctgttcctgagcatgcaagagggcaagaactacaccatctactgcaactacagcaccaccagcgaccggctgtactggtacagacaggatccaggcaagagcctggaaagcctgttcgtgctgctgagcaatggcgccgtgaaacaagagggtagactgatggccagcctggacaccaaggccagactgagcacactgcacattacagccgccgtgcacgatctgagcgccacctatttttgtgccggctactctggcgccggaagctaccagctgacatttggcaagggcaccaagctgagcgtgatcccc

SEQ ID NO: 214 - protein, 253 aa, synthetic construct
  REGION 1..253: note = TCR 37 Alpha Var. + Constant
ELKVEQNPLFLSMQEGKNYTIYCNYSTTSDRLYWYRQDPGKSLESLFVLLSNGAVKQEGRLMASLDTKARLSTLHITAAVHDLSATYFCAGYSGAGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 215 - protein, 112 aa, synthetic construct
  REGION 1..112: note = TCR 37 Alpha Var.
ELKVEQNPLFLSMQEGKNYTIYCNYSTTSDRLYWYRQDPGKSLESLFVLLSNGAVKQEGRLMASLDTKARLSTLHITAAVHDLSATYFCAGYSGAGSYQLTFGKGTKLSVIP

SEQ ID NO: 216 - DNA, 399 bp, synthetic construct
  misc_feature 1..399: note = TCR 37 Beta Var. + Constant
  source 1..399: mol_type = other DNA
atgggaacaagactgctgtgttgggtcgtgctgggcttcctgggcacagatcatacaggtgccggtgtctctcagagccccagatacaaggtggccaagcgcggacaggatgtggccctgagatgtgatcctatcagcggccacgtgtccctgttctggtatcagcaggctctcggacagggccccgagttcctgacctactttcagaatgaggcccagctggacaagagcggcctgcctagcgatagattcttcgccgaaagacccgagggcagcgtgtccacactgaagatccagagaacccagcaagaggacagcgccgtgtacctgtgtgcctctagtctgctgctgggcgcctacggctacacatttggctctggcaccagactgaccgtggtc

SEQ ID NO: 217 - protein, 291 aa, synthetic construct
  REGION 1..291: note = TCR 37 Beta Var. + Constant
GAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLLLGAYGYTFGSGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 218 - protein, 114 aa, synthetic construct
  REGION 1..114: note = TCR 37 Beta Var.
GAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLLLGAYGYTFGSGTRLTVV

SEQ ID NO: 219 - protein, 5 aa, synthetic construct
  REGION 1..5: note = TCR 37 Alpha CDR1
TTSDR

SEQ ID NO: 220 - protein, 7 aa, synthetic construct
  REGION 1..7: note = TCR 37 Alpha CDR2
LLSNGAV

SEQ ID NO: 221 - protein, 12 aa, synthetic construct
  REGION 1..12: note = TCR 37 Alpha CDR3
AGYSGAGSYQLT

SEQ ID NO: 222 - protein, 5 aa, synthetic construct
  REGION 1..5: note = TCR 37 Beta CDR1
SGHVS

SEQ ID NO: 223 - protein, 6 aa, synthetic construct
  REGION 1..6: note = TCR 37 Beta CDR2
FQNEAQ

SEQ ID NO: 224 - protein, 12 aa, synthetic construct
  REGION 1..12: note = TCR 37 Beta CDR3
ASSLLLGAYGYT

SEQ ID NO: 225 - DNA, 405 bp, synthetic construct
  misc_feature 1..405: note = TCR 49 Alpha Var. + Constant
  source 1..405: mol_type = other DNA
atgatgaagtccctgcgggtgctgctggtcatcctgtggctgcaactgagctgggtctggtcccagcagaaagaggtggaacagaacagcggccctctgtctgttcctgaaggcgctatcgccagcctgaactgcacctacagcgatagaggcagccagagcttcttctggtacagacagtacagcggcaagagccccgagctgatcatgttcatctacagcaacggcgacaaagaggacggccggtttacagcccagctgaacaaggccagccagtacgtgtccctgctgatcagagatagccagcctagcgacagcgccacctatctgtgcgccgtgaatatcggcaccagcggcacctacaagtacatcttcggcaccggcaccagactgaaggtgctggct

SEQ ID NO: 226 - protein, 254 aa, synthetic construct
  REGION 1..254: note = TCR 49 Alpha Var. + Constant
QKEVEQNSGPLSVPEGAIASLNCTYSDRGSQSFFWYRQYSGKSPELIMFIYSNGDKEDGRFTAQLNKASQYVSLLIRDSQPSDSATYLCAVNIGTSGTYKYIFGTGTRLKVLANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 227 - protein, 113 aa, synthetic construct
  REGION 1..113: note = TCR 49 Alpha Var.
QKEVEQNSGPLSVPEGAIASLNCTYSDRGSQSFFWYRQYSGKSPELIMFIYSNGDKEDGRFTAQLNKASQYVSLLIRDSQPSDSATYLCAVNIGTSGTYKYIFGTGTRLKVLA

SEQ ID NO: 228 - DNA, 396 bp, synthetic construct
  misc_feature 1..396: note = TCR 49 Beta Var. + Constant
  source 1..396: mol_type = other DNA
atggatacctggctcgtgtgctgggccatcttcagcctgctgaaagccggactgaccgagcctgaagtgacccagacacctagccaccaagtgacacagatgggccaagaagtgatcctgcgctgcgtgcccatcagcaaccacctgtacttctactggtacagacagatcctgggccagaaagtggaattcctggtgtccttctacaacaacgagatcagcgagaagtccgagatcttcgacgaccagttcagcgtggaaagacccgacggcagcaacttcaccctgaagatcagaagcaccaagctcgaggacagcgccatgtacttttgcgccagcacaccccggtacagctacgagcagtatttcggccctggcaccagactgaccgtgaca

SEQ ID NO: 229 - protein, 292 aa, synthetic construct
  REGION 1..292: note = TCR 49 Beta Var. + Constant
EPEVTQTPSHQVTQMGQEVILRCVPISNHLYFYWYRQILGQKVEFLVSFYNNEISEKSEIFDDQFSVERPDGSNFTLKIRSTKLEDSAMYFCASTPRYSYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 230 - protein, 113 aa, synthetic construct
  REGION 1..113: note = TCR 49 Beta Var.
EPEVTQTPSHQVTQMGQEVILRCVPISNHLYFYWYRQILGQKVEFLVSFYNNEISEKSEIFDDQFSVERPDGSNFTLKIRSTKLEDSAMYFCASTPRYSYEQYFGPGTRLTVT

SEQ ID NO: 231 - protein, 6 aa, synthetic construct
  REGION 1..6: note = TCR 49 Alpha CDR1
DRGSQS

SEQ ID NO: 232 - protein, 6 aa, synthetic construct
  REGION 1..6: note = TCR 49 Alpha CDR2
IYSNGD

SEQ ID NO: 233 - protein, 13 aa, synthetic construct
  REGION 1..13: note = TCR 49 Alpha CDR3
AVNIGTSGTYKYI

SEQ ID NO: 234 - protein, 5 aa, synthetic construct
  REGION 1..5: note = TCR 49 Beta CDR1
SNHLY

SEQ ID NO: 235 - protein, 6 aa, synthetic construct
  REGION 1..6: note = TCR 49 Beta CDR2
FYNNEI

SEQ ID NO: 236 - protein, 11 aa, synthetic construct
  REGION 1..11: note = TCR 49 Beta CDR3
ASTPRYSYEQY

SEQ ID NO: 237 - DNA, 402 bp, synthetic construct
  misc_feature 1..402: note = TCR 53 Alpha Var. + Constant
  source 1..402: mol_type = other DNA
atggccagcgctcctatctctatgctggccatgctgttcaccctgagcggactgagagcacagtctgtggcccagcctgaggaccaagtgaatgtggccgagggcaatcccctgaccgtgaagtgtacctacagcgtgtccggcaatccctacctgttttggtacgtgcagtaccccaaccggggcctgcagttcctgctgaagtacatcaccggcgacaacctggtcaagggcagctatggattcgaggccgagttcaacaagagccagaccagcttccacctgaagaaacccagcgctctggtgtccgatagcgccctgtatttctgcgccgtcagagattttggcagcggcacctacaagtacatcttcggcaccggcaccagactgaaggtgctggct

SEQ ID NO: 238 - protein, 256 aa, synthetic construct
  REGION 1..256: note = TCR 53 Alpha Var. + Constant
AQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDFGSGTYKYIFGTGTRLKVLANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 239 - protein, 115 aa, synthetic construct
  REGION 1..115: note = TCR 53 Alpha Var.
AQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDFGSGTYKYIFGTGTRLKVLA

SEQ ID NO: 240 - DNA, 404 bp, synthetic construct
  misc_feature 1..404: note = TCR 53 Beta Var. + Constant
  source 1..404: mol_type = other DNA
atgggcacaagcctgctgtgttgggtcgtgctgggctttctgggcacagatcatacaggcgccggtgtcagccagtctcctcggtacaaagtgaccaagcgcggacaggatgtggccctgagatgcgatcctatctctggccacgtgtccctgtactggtacagacaggctctcggacagggccccgagttcctgacctacttcaattacgaggcccagcaggacaagagcggcctgcctaacgatagattcagcgccgaaagacccgagggcagcatcagcacactgaccatccagagaaccgagcagagggacagcgccatgtacagatgtgcctctagcggcagcggcaccagcggctacaatgagcagtttttcggccctggcaccagactgacagtgct

SEQ ID NO: 241 - protein, 295 aa, synthetic construct
  REGION 1..295: note = TCR 53 Beta Var. + Constant
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSGSGTSGYNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 242 - protein, 116 aa, synthetic construct
  REGION 1..116: note = TCR 53 Beta Var.
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSGSGTSGYNEQFFGPGTRLTVL

SEQ ID NO: 243 - protein, 6 aa, synthetic construct
  REGION 1..6: note = TCR 53 Alpha CDR1
VSGNPY

SEQ ID NO: 244 - protein, 8 aa, synthetic construct
  REGION 1..8: note = TCR 53 Alpha CDR2
YITGDNLV

SEQ ID NO: 245 - protein, 13 aa, synthetic construct
  REGION 1..13: note = TCR 53 Alpha CDR3
AVRDFGSGTYKYI

SEQ ID NO: 246 - protein, 5 aa, synthetic construct
  REGION 1..5: note = TCR 53 Beta CDR1
SGHVS

SEQ ID NO: 247 - protein, 6 aa, synthetic construct
  REGION 1..6: note = TCR 53 Beta CDR2
FNYEAQ

SEQ ID NO: 248 - protein, 14 aa, synthetic construct
  REGION 1..14: note = TCR 53 Beta CDR3
ASSGSGTSGYNEQF

SEQ ID NO: 249 - protein, 246 aa, synthetic construct
  REGION 1..246: note = Anti-BCMA scFv
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 250 - protein, 244 aa, synthetic construct
  REGION 1..244: note = Anti-BCMA scFv
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 251 - DNA, 2256 bp, Homo sapiens
  misc_feature 1..2256: note = DGKalpha
  source 1..2256: mol_type = unassigned DNA
atggccaaggagaggggcctaataagccccagtgattttgcccagctgcaaaaatacatggaatactccaccaaaaaggtcagtgatgtcctaaagctcttcgaggatggcgagatggctaaatatgtccaaggagatgccattgggtacgagggattccagcaattcctgaaaatctatctcgaagtggataatgttcccagacacctaagcctggcactgtttcaatcctttgagactggtcactgcttaaatgagacaaatgtgacaaaagatgtggtgtgtctcaatgatgtttcctgctacttttcccttctggagggtggtcggccagaagacaagttagaattcaccttcaagctgtacgacacggacagaaatgggatcctggacagctcagaagtggacaaaattatcctacagatgatgcgagtggctgaatacctggattgggatgtgtctgagctgaggccgattcttcaggagatgatgaaagagattgactatgatggcagtggctctgtctctcaagctgagtgggtccgggctggggccaccaccgtgccactgctagtgctgctgggtctggagatgactctgaaggacgacggacagcacatgtggaggcccaagaggttccccagaccagtctactgcaatctgtgcgagtcaagcattggtcttggcaaacagggactgagctgtaacctctgtaagtacactgttcacgaccagtgtgccatgaaagccctgccttgtgaagtcagcacctatgccaagtctcggaaggacattggtgtccaatcacatgtgtgggtgcgaggaggctgtgagtccgggcgctgcgaccgctgtcagaaaaagatccggatctaccacagtctgaccgggctgcattgtgtatggtgccacctagagatccacgatgactgcctgcaagcggtgggccatgagtgtgactgtgggctgctccgggatcacatcctgcctccatcttccatctatcccagtgtcctggcctctggaccggatcgtaaaaatagcaaaacaagccagaagaccatggatgatttaaatttgagcacctctgaggctctgcggattgaccctgttcctaacacccacccacttctcgtctttgtcaatcctaagagtggcgggaagcaggggcagagggtgctctggaagttccagtatatattaaaccctcgacaggtgttcaacctcctaaaggatggtcctgagatagggctccgattattcaaggatgttcctgatagccggattttggtgtgtggtggagacggcacagtaggctggattctagagaccattgacaaagctaacttgccagttttgcctcctgttgctgtgttgcccctgggtactggaaatgatctggctcgatgcctaagatggggaggaggttatgaaggacagaatctggcaaagatcctcaaggatttagagatgagtaaagtggtacatatggatcgatggtctgtggaggtgatacctcaacaaactgaagaaaaaagtgacccagtcccctttcaaatcatcaataactacttctctattggcgtggatgcctctattgctcatcgattccacatcatgcgagagaaatatccggagaagttcaacagcagaatgaagaacaagctatggtacttcgaatttgccacatctgaatccatcttctcaacatgcaaaaagctggaggagtctttgacagttgagatctgtgggaaaccgctggatctgagcaacctgtccctagaaggcatcgcagtgctaaacatccctagcatgcatggtggctccaacctctggggtgataccaggagaccccatggggatatctatgggatcaaccaggccttaggtgctacagctaaagtcatcaccgaccctgatatcctgaaaacctgtgtaccagacctaagtgacaagagactggaagtggttgggctggagggtgcaattgagatgggccaaatctataccaagctcaagaatgctggacgtcggctggccaagtgctctgagatcaccttccacaccacaaaaacccttcccatgcaaattgacggagaaccctggatgcagacgccctgtacaatcaagatcacccacaagaaccagatgcccatgctcatgggcccacccccccgctccaccaatttctttggcttcttgagcggatcctcggagacagtgcggtttcagggacaccaccaccatcaccactga

SEQ ID NO: 252 - DNA, 2838 bp, Homo sapiens
  misc_feature 1..2838: note = DGKzeta
  source 1..2838: mol_type = unassigned DNA
atggagccgcgggacggtagccccgaggcccggagcagcgactccgagtcggcttccgcctcgtccagcggctccgagcgcgacgccggtcccgagccggacaaggcgccgcggcgactcaacaagcggcgcttcccggggctgcggctcttcgggcacaggaaagccatcacgaagtcgggcctccagcacctggccccccctccgcccacccctggggccccgtgcagcgagtcagagcggcagatccggagtacagtggactggagcgagtcagcgacatatggggagcacatctggttcgagaccaacgtgtccggggacttctgctacgttggggagcagtactgtgtagccaggatgctgcagaagtcagtgtctcgaagaaagtgcgcagcctgcaagattgtggtgcacacgccctgcatcgagcagctggagaagataaatttccgctgtaagccgtccttccgtgaatcaggctccaggaatgtccgcgagccaacctttgtacggcaccactgggtacacagacgacgccaggacggcaagtgtcggcactgtgggaagggattccagcagaagttcaccttccacagcaaggagattgtggccatcagctgctcgtggtgcaagcaggcataccacagcaaggtgtcctgcttcatgctgcagcagatcgaggagccgtgctcgctgggggtccacgcagccgtggtcatcccgcccacctggatcctccgcgcccggaggccccagaatactctgaaagcaagcaagaagaagaagagggcatccttcaagaggaagtccagcaagaaagggcctgaggagggccgctggagacccttcatcatcaggcccaccccctccccgctcatgaagcccctgctggtgtttgtgaaccccaagagtgggggcaaccagggtgcaaagatcatccagtctttcctctggtatctcaatccccgacaagtcttcgacctgagccagggagggcccaaggaggcgctggagatgtaccgcaaagtgcacaacctgcggatcctggcgtgcgggggcgacggcacggtgggctggatcctctccaccctggaccagctacgcctgaagccgccaccccctgttgccatcctgcccctgggtactggcaacgacttggcccgaaccctcaactggggtgggggctacacagatgagcctgtgtccaagatcctctcccacgtggaggaggggaacgtggtacagctggaccgctgggacctccacgctgagcccaaccccgaggcagggcctgaggaccgagatgaaggcgccaccgaccggttgcccctggatgtcttcaacaactacttcagcctgggctttgacgcccacgtcaccctggagttccacgagtctcgagaggccaacccagagaaattcaacagccgctttcggaataagatgttctacgccgggacagctttctctgacttcctgatgggcagctccaaggacctggccaagcacatccgagtggtgtgtgatggaatggacttgactcccaagatccaggacctgaaaccccagtgtgttgttttcctgaacatccccaggtactgtgcgggcaccatgccctggggccaccctggggagcaccacgactttgagccccagcggcatgacgacggctacctcgaggtcattggcttcaccatgacgtcgttggccgcgctgcaggtgggcggacacggcgagcggctgacgcagtgtcgcgaggtggtgctcaccacatccaaggccatcccggtgcaggtggatggcgagccctgcaagcttgcagcctcacgcatccgcatcgccctgcgcaaccaggccaccatggtgcagaaggccaagcggcggagcgccgcccccctgcacagcgaccagcagccggtgccagagcagttgcgcatccaggtgagtcgcgtcagcatgcacgactatgaggccctgcactacgacaaggagcagctcaaggaggcctctgtgccgctgggcactgtggtggtcccaggagacagtgacctagagctctgccgtgcccacattgagagactccagcaggagcccgatggtgctggagccaagtccccgacatgccagaaactgtcccccaagtggtgcttcctggacgccaccactgccagccgcttctacaggatcgaccgagcccaggagcacctcaactatgtgactgagatcgcacaggatgagatttatatcctggaccctgagctgctgggggcatcggcccggcctgacctcccaacccccacttcccctctccccacctcaccctgctcacccacgccccggtcactgcaaggggatgctgcaccccctcaaggtgaagagctgattgaggctgccaagaggaacgacttctgtaagctccaggagctgcaccgagctgggggcgacctcatgcaccgagacgagcagagtcgcacgctcctgcaccacgcagtcagcactggcagcaaggatgtggtccgctacctgctggaccacgcccccccagagatccttgatgcggtggaggaaaacggggagacctgtttgcaccaagcagcggccctgggccagcgcaccatctgccactacatcgtggaggccggggcctcgctcatgaagacagaccagcagggcgacactccccggcagcgggctgagaaggctcaggacaccgagctggccgcctacctggagaaccggcagcactaccagatgatccagcgggaggaccaggagacggctgtgggatcctcggagacagtgcggtttcagggacaccaccaccatcaccactga